(19) United States
(12) Patent Application Publication
(10) Pub. No.: US 2021/0260178 A1
(43) Pub. Date: Aug. 26, 2021

(54) NOVEL LASSA VIRUS RNA MOLECULES AND COMPOSITIONS FOR VACCINATION

(71) Applicant: CureVac AG, Tübingen (DE)

(72) Inventors: Edith JASNY, Tübingen (DE); Benjamin PETSCH, Tübingen (DE); Hans Wolfgang GROSSE, Tübingen (DE)

(73) Assignee: CureVac AG, Tübingen (DE)

(21) Appl. No.: 17/254,847

(22) PCT Filed: Jun. 27, 2019
(86) PCT No.: PCT/EP2019/067205

§ 371 Date: Dec. 21, 2020

(30) Foreign Application Priority Data
Jun. 27, 2018 (EP) PCT/EP2018/067330

(51) Int. Cl. A61K 39/12; C07K 14/005; C12N 7/00; A61P 31/14

(52) U.S. Cl. CPC A61K 39/12; C07K 14/005; C12N 7/00; A61K 2039/53; C12N 2760/10022; C12N 2760/10034; A61P 31/14

(57) ABSTRACT

The present invention is directed to RNA suitable for use in treatment and/or prophylaxis of an infection with Lassa virus (LASV) or of a disorder related to such an infection. The invention further concerns a method of treating or preventing a disorder or a disease, first and second medical uses of the RNA, compositions, and vaccines. Further, the invention is directed to a kit, particularly to a kit of parts, comprising the RNA, compositions and vaccines.

### INTRODUCTION

The present invention is directed to an RNA suitable for use in the treatment or prophylaxis of an infection with Lassa virus (LASV) or of a disorder related to such an infection. In particular, the RNA of the invention comprises at least one heterologous untranslated region (UTR), preferably a 3′-UTR and/or a 5′-UTR, and a coding sequence encoding at least one antigenic peptide or protein derived from LASV glycoprotein precursor (GPC), prefusion-stabilized GPC, LASV nucleoprotein (NP), or LASV zinc-binding matrix protein (Z). The RNA is preferably characterized by increased expression efficacies of said coding sequences operably linked to said advantageous UTR elements. The invention is also directed to compositions and vaccines comprising said RNA in association or in complexation with a polymeric carrier, a polycationic protein or peptide, or a lipid nanoparticle (LNP). Further, the invention concerns a kit, particularly a kit of parts comprising the RNA or composition or vaccine. The invention is further directed to a method of treating or preventing a disorder or a disease, and first and second medical uses of the RNA, composition, or vaccine.

Lassa virus, or Lassa mammarenavirus (LASV), belongs to the Arenaviridae, a family of enveloped viruses with bi-segmented, single-stranded RNA genomes. The large (L) genomic segment encodes an RNA-dependent RNA polymerase (RdRp) and a small RING finger z protein (analogous of matrix protein). The small (S) genomic segment encodes the nucleoprotein (NP), and the glycoprotein precursor (GPC). GPC is translated as single polypeptide precursor and undergoes processing by signal peptidases and cellular pro-protein convertases yielding the stable signal peptide (SSP), the N-terminal GP1, and the transmembrane GP2. The virion form of GPC is a trimer of heterodimers, each containing the N-terminal subunit GP1 and the transmembrane fusion-mediating subunit GP2. GPC represents the sole antigen on the LASV surface and is therefore considered the primary target of protective humoral immune responses.

LASV is endemic in West Africa with estimated 100,000-300,000 infections and 5,000-10,000 deaths annually. The early stage of human LASV infection is characterized by unspecific symptoms. After an incubation period of about 3-21 days, early symptoms include fever, sore throat, retrosternal pain, and myalgia. In progressed patients, elevated liver enzymes and high viral load in plasma (viremia) combined with vascular manifestations are indicators of poor prognosis and a fatal outcome. Human infection occurs via contact with rodents (e.g., Mastomys natalensis), inhalation of contaminated droplets/dust, ingestion of contaminated food, or contact with infected patients. Importantly, adaptive cellular immune response seems to play a key role in outcome of LASV infection in humans. Growing body of evidence indicates that very early events during the natural infection can affect the balance between effective adaptive immune responses and virus replication and determine the clinical outcome. T cell responses seem to be central for immunity to LASV. In humans, strong CD4+ T cell memory responses against LASV NP can be recalled in PBMCs for up to six years after the initial infection, and T cell memory responses to GPC are similarly long-lived. LASV infection that results in a fatal outcome is associated with a lack of demonstrable T cell activation.

Accordingly, provision of a LASV vaccine is an imperative public health need. In the art, several approaches for developing a LASV vaccine exist that are summarized in the following.

A potential approach includes epitope-based vaccines comprising e.g. HLA-binding LASV peptides. However, there are serious safety concerns regarding peptide-based vaccination of individuals recently infected with the virus or in immune individuals previously exposed to the pathogen due to the potential reactivation of CD8+ memory cell progeny leading to potentially fatal immune reactions.

Another potential approach includes virus-based vaccine platforms e.g. alphavirus vector-based vaccines or recombinant vaccinia virus-based vaccines expressing LASV antigens. However, recombinant replication-defective viruses may exhibit a low immunogenicity and thus insufficient protection, and viruses exhibiting an immunosuppressive phenotype such as e.g. vaccinia virus, harboring the risk of reversion to virulence.

Another challenge associated with past and future LASV vaccine development is the great genetic diversity among LASV strains. Recent studies using next-generation sequencing showed LASV clustering into four major clades and provided further evidence for high LASV genome diversity. Moreover, LASV in vivo re-assortment has been documented suggesting infection of individual hosts with at least two LASV strains from distinguished clades.

As outlined above, there is currently no licensed LASV vaccine and vaccine development is hampered by high cost of biocontainment requirement, the absence of appropriate small animal models and the genetic diversity of LASV species. LASV vaccine platforms currently under development have been falling short of expectations in terms of safety and efficacy. Accordingly, there remains an unmet medical need for an efficient vaccine for prophylaxis or treatment of LASV infections.

Accordingly, it is the object of the underlying invention to provide novel RNA coding for antigenic peptides or proteins of LASV and compositions/vaccines comprising said RNA for the use as vaccine for prophylaxis or treatment of LASV infections.

Further it would be desirable that an RNA-based composition or vaccine has some of the following advantageous features:
Improved translation of RNA constructs at the site of injection (e.g. muscle).
Very efficient induction of LASV antigen-specific immune responses against the encoded antigenic peptide or protein at a low dosages and dosing regimen.
Suitability for intramuscular administration.
Induction of an LASV-specific functional humoral immune response.
Induction of an LASV-specific neutralizing antibody immune response.
Induction of LASV-specific B-cell memory.
Faster onset of immune protection against LASV.
Longevity of the induced immune responses against LASV.
Induction of broad T-cell responses against LASV.
Induction of a (local and transient) pro-inflammatory environment.
No induction of systemic cytokine or chemokine response after application of the vaccine.
Good tolerability, no side-effects, non toxic effects.
Advantageous stability characteristics of the RNA, the composition and the vaccine.
Speed, adaptability, simplicity and scalability of LASV vaccine production.
Protection against a broad spectrum of LASV strains.
Formulation(s) and production compatible with many different antigens.

The objects outlined above are solved by the claimed subject matter of the independent claims, wherein further embodiments are incorporated in the dependent claims.

Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, the antigen is provided by the RNA coding sequence encoding at least one antigenic peptide or protein.

Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins derived from LASV comprising at least one epitope may be understood as antigens. In the context of the present invention, an antigen may be the product of translation of a provided RNA of the first aspect.

Antigenic peptide or protein: The term “antigenic peptide or protein” will be recognized and understood by the person of ordinary skill in the art, and is e.g. intended to refer to a peptide or protein derived from a (antigenic) protein which may stimulate the body's adaptive immune system to provide an adaptive immune response. Therefore an “antigenic peptide or protein” comprises at least one epitope (as defined herein) or antigen (as defined herein) of the protein it is derived from (i.e., a LASV antigenic peptide or protein).

Artificial RNA: The term “artificial RNA” as used herein is intended to refer to an RNA that does not occur naturally. In other words, an artificial RNA may be understood as a non-natural nucleic acid molecule. Such RNA molecules may be non-natural due to its individual sequence (e.g. G/C content modified coding sequence, UTRs) and/or due to other modifications, e.g. structural modifications of nucleotides. Typically, artificial RNA may be designed and/or generated by genetic engineering to correspond to a desired artificial sequence of nucleotides (i.e., heterologous sequence). In this context an artificial RNA is a sequence that may not occur naturally, i.e. it differs from the wild type sequence by at least one nucleotide. The term “artificial RNA” is not restricted to mean “one single molecule” but is understood to comprise an ensemble of essentially identical molecules. Accordingly, it may relate to a plurality of essentially identical RNA molecules. The RNA of the invention is preferably an artificial RNA.

Cationic, cationisable: Unless a different meaning is clear from the specific context, the term “cationic” means that the respective structure bears a positive charge, either permanently or not permanently but in response to certain conditions such as e.g. pH. Thus, the term “cationic” covers both “permanently cationic” and “cationisable”. The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Cellular immunity/cellular immune response: “Cellular immunity”, “cellular immunity”, “cellular immune response” or “cellular T-cell responses” relates to the activation of macrophages, natural killer cells, antigen- specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface. Such cells may be virus-infected or infected with intracellular bacteria, or cancer cells displaying tumor antigens. Further characteristics may be activation of macrophages and natural killer cells, enabling them to destroy pathogens and stimulation of cells to secrete a variety of cytokines that influence the function of other cells involved in adaptive immune responses and innate immune responses. In the context of the invention, the antigen is provided by the RNA of the first aspect, suitably inducing T-cell responses against LASV antigens (e.g. LASV NP).

Coding sequence/coding region: The terms “coding sequence” or “coding region” and “cds” as used herein will be recognized and understood by the person of ordinary skill in the art, and are e.g. intended to refer to a sequence of several nucleotides which may be translated into a peptide or protein. In the context of the present invention a cds is preferably an RNA sequence, consisting of a number of nucleotide triplets, starting with a start codon and preferably terminating with a stop codon.

Composition: In the context of the invention, a “composition” refers to any type of composition in which the specified ingredients (e.g. RNA e.g. in association with LNP), may be incorporated, optionally along with any further constituents, usually with at least one pharmaceutically acceptable carrier or excipient. Thus, the composition may be a dry composition such as a powder or granules, or a solid unit such as a lyophilized form or a tablet. Alternatively, the composition may be in liquid form, and each constituent may be independently incorporated in dissolved or dispersed (e.g. suspended or emulsified) form.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Epitope: The term “epitope” as used herein will be recognized and understood by the person of ordinary skill in the art, and is e.g. intended to refer to T cell and B cell epitopes. T cell epitopes or parts of the antigenic peptides or proteins may comprise fragments preferably having a length of about 6 to about 20 or even more amino acids (aa), e.g. fragments as processed and presented by MHC class I molecules have a typical length of about 8 aa to about 10 aa, or fragments as processed and presented by MHC class II molecules have a typical length of about 13 aa to about 20 aa. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens typically having a length of about 5 aa to about 15 aa which may be recognized by antibodies. Such epitopes of proteins or peptides may furthermore be selected from any of the herein mentioned variants of such proteins or peptides. In this context epitopes can be conformational or discontinuous which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain. In the context of the present invention, an epitope may be the product of translation (and subsequent post-translational modification and processing) of a provided RNA.

Fragment: The term “fragment” as used throughout the present specification in the context of a nucleic acid sequence or an amino acid (aa) sequence may typically be a shorter portion of a full-length sequence of e.g. a nucleic acid sequence or an amino acid sequence. A fragment typically consists of a sequence that is identical to the corresponding stretch within the full-length sequence. The term “fragment” as used throughout the present specification in the context of proteins or peptides may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the aa level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of antigenic proteins or peptides may comprise at least one epitope of those proteins or peptides. Furthermore also domains of a protein, like the extracellular domain, the intracellular domain or the transmembrane domain and shortened or truncated versions of a protein may be understood to comprise a fragment of a protein.

Heterologous: The terms “heterologous” or “heterologous sequence” as used throughout the present specification in the context of a nucleic acid sequence or an amino acid sequence refers to a sequence (e.g. DNA, RNA, amino acid) will be recognized and understood by the person of ordinary skill in the art, and is intended to refer to a sequence that is derived from another gene, from another allele, from another species. Two sequences are typically understood to be “heterologous” if they are not derivable from the same gene or in the same allele. I.e., although heterologous sequences may be derivable from the same organism, they naturally (in nature) do not occur in the same nucleic acid molecule, such as e.g. in the same RNA or protein.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or aa sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Immunogen, immunogenic: The terms “immunogen” or “immunogenic” will be recognized and understood by the person of ordinary skill in the art, and are e.g. intended to refer to a compound that is able to stimulate/induce an immune response. Preferably, an immunogen is a peptide, or protein. An immunogen in the sense of the present invention is the product of translation of a provided RNA.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is e.g. intended to refer to a system of an organism that may protect said organisms from infection. If a pathogen enters an organism, the innate immune system provides an immediate, non-specific response. The adaptive immune system adapts its response during an infection to improve recognition of the pathogen. This improved response is then retained after the pathogen has been eliminated, in the form of an immunological memory, allowing for faster and stronger attacks each time that pathogen is encountered.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be, e.g. activated by ligands of Toll-like receptors (TLRs) or other auxiliary substances such as lipopolysaccharides, TNF-alpha, CD40 ligand, or cytokines, monokines, lymphokines, interleukins or chemokines, IL-1 to IL-33, IFN-alpha, IFN-beta, IFN-gamma, GM-CSF, G-CSF, M-CSF, LT-beta, TNF-alpha, growth factors, and hGH, a ligand of human TLR1 to TLR10, a ligand of murine TLR1 to TLR13 a ligand of a NOD-like receptor, a ligand of a RIG-I like receptor, an immunostimulatory nucleic acid, an immunostimulatory RNA (isRNA), a CpG-DNA etc.

Monovalent vaccine, monovalent composition: The terms “monovalent vaccine”, “monovalent composition” “univalent vaccine” or “univalent composition” will be recognized and understood by the person of ordinary skill in the art, and are e.g. intended to refer to a composition or a vaccine comprising only one antigen from a virus. Accordingly, said vaccine or composition comprises only one RNA species encoding a single antigen for a single organism. The term “monovalent vaccine” includes the immunization against a single valence. In the context of the invention, a monovalent LASV vaccine or composition would comprise an RNA encoding one single antigenic peptide or protein derived from one LASV.

Nucleic acid: The terms “nucleic acid” or “nucleic acid molecule” will be recognized and understood by the person of ordinary skill in the art, and are e.g. intended to refer to a molecule comprising, preferably consisting of nucleic acid components. The term nucleic acid molecule preferably refers to DNA or RNA. It is preferably used synonymous with the term polynucleotide. Preferably, a nucleic acid or a nucleic acid molecule is a polymer comprising or consisting of nucleotide monomers (natural and/or modified), which are covalently linked to each other by phosphodiester-bonds of a sugar/phosphate-backbone. The term “nucleic acid molecule” also encompasses modified nucleic acid molecules, such as base-modified, sugar-modified or backbone-modified DNA or RNA molecules as defined herein.

Nucleic acid sequence/ RNA sequence/ amino acid sequence: The terms “nucleic acid sequence”, “RNA sequence” or “amino acid sequence” will be recognized and understood by the person of ordinary skill in the art, and are e.g. intended to refer to particular and individual order of the succession of its nucleotides or amino acids respectively.

Pharmaceutically effective amount: The terms “pharmaceutically effective amount” or “effective amount” will be recognized and understood by the person of ordinary skill in the art, and are e.g. intended to refer to an amount of a compound sufficient to induce a pharmaceutical effect, i.e. an immune response.

Polyvalent/multivalent vaccine, polyvalent/multivalent composition: The terms “polyvalent vaccine”, “polyvalent composition” “multivalent vaccine” or “multivalent composition” will be recognized and understood by the person of ordinary skill in the art, and are e.g. intended to refer to a composition or a vaccine comprising antigens from more than one strain of a virus, or comprising different antigens of the same virus, or any combination thereof. The terms describe that said vaccine or composition has more than one valence. In the context of the invention, a polyvalent LASV vaccine would comprise an RNA encoding antigenic peptides or proteins derived from several different LASV strains and/or clades or an RNA encoding different antigens from the same LASV strain, or a combination thereof.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides will be recognized and understood by the person of ordinary skill in the art, and is e.g. intended to refer to a proteins or peptide variant having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined herein may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, e.g., an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means that the variant exerts the same effect or functionality or at least 40%, 50%, 60%, 70%, 80%, 90%, or 95% of the effect or functionality as the protein it is derived from.

3′-untranslated region, 3′-UTR element, 3′-UTR: The terms “3′-untranslated region” or “3′-UTR” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of an mRNA located between a cds and a terminal poly(A) sequence. A 3′-UTR may comprise elements for controlling gene expression, also called regulatory elements. Such regulatory elements may be, e.g., ribosomal binding sites, miRNA binding sites etc.

5′-untranslated region, 5′-UTR element, 5′-UTR: The terms “5′-untranslated region” or “3′-UTR” will be recognized and understood by the person of ordinary skill in the art, and are e.g. intended to refer to a part of a nucleic acid molecule located 5′ (i.e. “upstream”) of a coding sequence and which is not translated into protein. A 5′-UTR may be part of an mRNA located 5′ of the cds. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic addition of a 5′-cap structure.

5′-terminal oligopyrimidine tract (TOP), TOP-UTR: The term “5′-terminal oligopyrimidine tract (TOP)” has to be understood as a stretch of pyrimidine nucleotides located in the 5′-terminal region of a nucleic acid molecule, such as the 5′-terminal region of certain mRNA molecules or the 5′-terminal region of a functional entity, e.g. the transcribed region of certain genes. The sequence starts with a cytidine, which usually corresponds to the transcriptional start site, and is followed by a stretch of usually about 3 to 30 pyrimidine nucleotides. A TOP may e.g. comprise 3-30 or even more nucleotides. The pyrimidine stretch and thus the 5′-TOP ends one nucleotide 5′ to the first purine nucleotide located downstream of the TOP. mRNA that contains a 5′-terminal oligopyrimidine tract is often referred to as TOP mRNA. Accordingly, genes that provide such mRNAs are referred to as TOP genes. The term “TOP motif” or “5′-TOP motif” has to be understood as a nucleic acid sequence which corresponds to a 5′-TOP as defined above. Thus, a TOP motif in the context of the present invention is preferably a stretch of pyrimidine nucleotides having a length of 3-30 nucleotides. The TOP-motif may consist of at least 3, 4, 5, 6, 7, or 8 pyrimidine nucleotides, wherein the stretch of pyrimidine nucleotides may start at its 5′-end with a cytosine nucleotide. In TOP genes and TOP mRNAs, the TOP-motif may start at its 5′-end with the transcriptional start site and ends one nucleotide 5′ to the first purine residue in said gene or mRNA. A TOP motif may be located at the 5′-end of a sequence which represents a 5′-UTR or at the 5′-end of a sequence which codes for a 5′-UTR. A stretch of 3 or more pyrimidine nucleotides may be called “TOP motif” if this stretch is located at the 5′-end of a respective sequence, such as the nucleic acid, the 5′-UTR element of the nucleic acid, or the nucleic acid sequence which is derived from the 5′-UTR of a TOP gene as described herein. The nucleic acid sequence of the 5′-UTR element, which is derived from a 5′-UTR of a TOP gene, may also terminate at its 3′-end with a nucleotide located at position 1, 2, 3, 4, 5, 6, 7, 8, 9 or 10 upstream of the start codon of the gene or RNA it is derived from.

### Short Description of the Invention

The present invention is based on the surprising finding that at least one peptide or protein derived from of a Lassa virus (LASV) glycoprotein precursor (GPC), nucleoprotein (NP), or zinc-binding matrix protein (Z) encoded by the RNA of the invention can efficiently be expressed in a mammalian cell. Even more unexpected, the inventors showed that the RNA of the invention can induce antigen-specific functional immune responses, e.g. effective cellular and humoral responses. Through optimizations of LASV antigen design (e.g. prefusion-stabilized GPC), the immune responses could be further improved. In addition, the expression of the LASV antigen encoded by the RNA could be increased via suitable heterologous 5′ untranslated regions (UTRs) and suitable heterologous 3′ untranslated regions (UTRs). Advantageously, said RNA comprising suitable 3′-UTR/5′-UTR combinations induce very efficient antigen-specific immune responses against the encoded LASV antigenic protein. Further, the RNA of the invention comprised in lipid nanoparticles (LNPs) very efficiently induces antigen-specific immune responses against LASV protein at a low dosages and dosing regimen. Furthermore, RNAs encoding different antigens (e.g GPC and NP, optionally derived from different LASV viruses) can be effectively combined in one RNA-based vaccine, e.g. to improve T-cell responses and/or to induce virus-like particle formation and/or to confer broad protection against different LASV clades. The RNA and the composition/vaccine comprising said RNA is therefore suitable for use as a vaccine, e.g. as a human vaccine. Advantageously, the RNA according to the invention enables rapid and rational vaccine design with flexibility, speed, and scalability of production.

In a first aspect, the present invention provides an RNA comprising at least one coding sequence encoding at least one antigenic peptide or protein derived from a Lassa virus (LASV) protein or a fragment or variant thereof, wherein said coding sequence is operably linked to a 5′-UTR derived from a HSD17B4 gene, a NDUFA4 gene, or a RPL32 gene and/or a 3′-UTR derived from a PSMB3 gene, a CASP1 gene, an ALB7 gene, or an alpha-globin gene.

The at least one antigenic peptide or protein may suitably be derived from LASV glycoprotein precursor (GPC), LASV nucleoprotein (NP), LASV zinc-binding matrix protein (Z), or a variant, fragment, or combination thereof, wherein GPC, NP, Z are preferably full-length proteins.

The at least one antigenic peptide or protein may suitably be derived from a GPC protein comprising a mutation to stabilize prefusion conformation, herein referred to as “prefusion-stabilized GPC” or “GPCstabilized”.

The RNA may comprise a codon modified coding sequence selected from C maximized coding sequence, CAI maximized coding sequence, human codon usage adapted coding sequence, G/C content modified coding sequence, and G/C optimized coding sequence, or any combination thereof.

Preferably, the RNA may comprise a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 255-2286, 3821-6106, 7798-9805, 11348-12803, 18002-18103, 19362-19463, 20723-20824, 22981-23108, 24677-24804, 26373-26500, 28069-28196, 29765-29892, 31461-31588, 33157-33284, 34853-34980, 36549-36676, 38245-38372, 41637-41764 or a fragment or variant of any of these sequences.

The RNA may further comprise at least one selected from a cap structure, a poly(A) sequence, a poly(C)sequence, a histone-stem loop, and/or a 3′-terminal sequence element.

The RNA preferably comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2287-3566, 14056-15207 (encoding GPC) or a fragment or variant of any of these, SEQ ID NOs: 6107-7546, 15208-16743, 23109-24644, 24805-26340, 26501-28036, 28197-29732, 29893-31428, 31589-33124, 33285-34820, 34981-36516, 36677-38212, 38373-39908, 40069-41604, 41765-43300 (encoding prefusion-stabilized GPC) or a fragment or variant of any of these, SEQ ID NOs: 9806-11165, 16744-17967, 18104-19327, 19464-20687, 20825-22048 (encoding NP or SP-NP) or a fragment or variant of any of these, SEQ ID NOs: 12804-13803, 22049-22948 (encoding Z) or a fragment or variant of any of these.

In a second aspect, the present invention provides a composition comprising at least one or more than one or a plurality, e.g. 2, 3, 4, 5, 6, 7, 8, 9, 10, 11, 12, 13, 14, 15 of the RNAs of the first aspect.

The composition may comprises at least one RNA encoding at least one antigenic peptide or protein derived from GPC or prefusion-stabilized GPC or a variant or fragment thereof and at least one additional RNA encoding at least one antigenic peptide or protein derived from NP or a variant or fragment thereof. Advantageously, NP may promote efficient T-cell responses of the composition or vaccine when administered to a subject.

The composition may comprise at least one RNA encoding at least one antigenic peptide or protein derived from GPC or prefusion-stabilized GPC or a variant or fragment thereof and at least one additional RNA encoding at least one antigenic peptide or protein derived from NP or a variant or fragment thereof and at least one additional RNA encoding at least one antigenic peptide or protein derived from Z or a variant or fragment thereof. Advantageously, GPC, NP and Z may promote the formation of virus-like particles (VLP).

In embodiments, antigenic peptides or proteins may be derived from the same LASV or from different LASV or combinations thereof, wherein the different LASV belong to different LASV clades or different LASV lineages, preferably to the LASV clades I, II, III and IV or to the LASV lineages I, II, III and IV.

The composition may preferably comprise the an RNA of the invention complexed with one or more lipids thereby forming lipid nanoparticles (LNP), wherein the LNP essentially consists of

(i) at least one cationic lipid as defined herein, preferably a lipid of formula (III), more preferably lipid III-3;

(ii) a neutral lipid as defined herein, preferably 1,2-distearoyl-sn-glycero-3-phosphocholine (DSPC);

(iii) a steroid or steroid analogue as defined herein, preferably cholesterol; and

(iv) a PEG-lipid as defined herein, e.g. PEG-DMG or PEG-cDMA, preferably a PEGylated lipid of formula (IVa);

wherein (i) to (iv) are in a molar ratio of about 20-60% cationic lipid: 5-25% neutral lipid: 25-55% sterol; 0.5-15% PEG-lipid.

In a third aspect, the invention provides a LASV vaccine comprising the RNA of the first aspect or the composition of the second aspect.

In a fourth aspect, the invention provides a kit, particularly a kit of parts, comprising the RNA, compositions and vaccines.

In further aspects, the invention relates to the first and second medical use of the RNA, the composition, the vaccine, the kit or kit of parts and to a method of treating or preventing an infection with a LASV, or a disorder related to such an infection.

### DETAILED DESCRIPTION OF THE INVENTION

The present application is filed together with a sequence listing in electronic format, which is part of the description of the present application (WIPO standard ST.25). The information contained in the electronic format of the sequence listing filed together with this application is incorporated herein by reference in its entirety. For many sequences, the sequence listing also provides additional detailed information, e.g. regarding certain structural features, sequence optimizations, GenBank identifiers, or additional detailed information regarding its coding capacity. In particular, such information is provided under numeric identifier <223> in the WIPO standard ST.25 sequence listing. Accordingly, information provided under said numeric identifier <223> is explicitly included herein in its entirety and has to be understood as integral part of the description of the underlying invention.

RNA:

In a first aspect, the invention relates to an RNA comprising
a) at least one heterologous 5′ untranslated region (5′-UTR) and/or at least one heterologous 3′ untranslated region (3′-UTR); and
b) at least one coding sequence (or coding region) operably linked to said 3′-UTR and/or 5′-UTR encoding at least one antigenic peptide or protein derived from a LASV protein or a fragment or variant thereof.

The RNA of the invention may be composed of a protein-coding region (“coding sequence” or “cds”), and 5′- and/or 3′-UTR. Notably, UTRs may harbor regulatory sequence elements that determine RNA turnover, stability, and localization. Moreover, UTRs may harbor sequence elements that enhance translation. In medical application of RNA, translation of said RNA into protein is of paramount importance to therapeutic efficacy. The inventors surprisingly found that certain combinations of 3′-UTRs and/or 5′-UTRs enhance the expression of operably linked coding sequences encoding LASV antigenic peptides or proteins. RNA molecules harboring said UTR combinations advantageously enable rapid and transient expression of LASV antigenic peptides or proteins. Accordingly, the RNA provided herein is particularly suitable for vaccination against LASV.

Suitably, the RNA of the first aspect may comprise at least one heterologous 5′-UTR and/or at least one heterologous 3′-UTR. Said heterologous 5′-UTRs or 3′-UTRs may be derived from naturally occurring genes or may be synthetically engineered.

In preferred embodiments, the at least one RNA comprises at least one heterologous 3′-UTR.

Preferably the RNA comprises a 3′-UTR, which may be derivable from a gene that relates to an RNA with enhanced half-life (i.e. that provides a stable RNA).

In preferred embodiments of the first aspect, the RNA comprises at least one heterologous 3′-UTR, wherein the at least one heterologous 3′-UTR comprises a nucleic acid sequence derived from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes. Particularly preferred 3′-UTRs are PSMB3, CASP1, ALB7, or muag.

ALB7-derived 3′-UTR: The RNA may comprise a 3′-UTR which is derived from the 3′-UTR of a vertebrate albumin gene or from a variant thereof, preferably from the 3′-UTR of a mammalian albumin gene or from a variant thereof, more preferably from the 3′-UTR of a human albumin gene or from a variant thereof, even more preferably from the 3′-UTR of the human albumin gene, or from a homolog, fragment or variant thereof. Accordingly, the RNA may comprise a 3′-UTR derived from a ALB7 gene, wherein said 3′-UTR derived from an ALB7 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13838 or 13839 or a fragment or a variant thereof.

Alpha-globin gene -derived 3′-UTR: The RNA may comprise a 3′-UTR which is derived from the 3′-UTR of a vertebrate alpha-globin gene (referred to as “muag”) or from a variant thereof, preferably from the 3′-UTR of a mammalian alpha-globin or from a variant thereof, more preferably from the 3′-UTR of a human alpha-globin gene or from a variant thereof, even more preferably from the 3′-UTR of the human alpha-globin gene.

Accordingly, the RNA may comprise a 3′-UTR derived from a alpha-globin gene, wherein said 3′-UTR derived from a alpha-globin gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13840 or 13841 or a fragment or a variant thereof.

PSMB3-derived 3′-UTR: The RNA may comprise a 3′-UTR which is derived from a 3′-UTR of a gene encoding a proteasome subunit beta type-3 (PSMB3) protein, or a homolog, variant, fragment or derivative thereof. Such 3′-UTRs preferably comprise or consist of a nucleic acid sequences derived from the 3′-UTR of a proteasome subunit beta type-3 (PSMB3) gene, preferably from a vertebrate, more preferably a mammalian, most preferably a human proteasome subunit beta type-3 (PSMB3) gene, or a homolog, variant, fragment or derivative thereof. Accordingly, the RNA may comprise a 3′-UTR derived from a PSMB3 gene, wherein said 3′-UTR derived from a PSMB3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13826 or 13827 or a fragment or a variant thereof.

CASP1-derived 3′-UTR: The RNA may comprise a 3′-UTR which is derived from a 3′-UTR of a gene encoding a Caspase-1 (CASP1) protein, or a homolog, variant, fragment or derivative thereof. Such 3′-UTRs preferably comprise or consist of a nucleic acid sequence derived from the 3′-UTR of a Caspase-1 (CASP1) gene, preferably from a vertebrate, more preferably a mammalian, most preferably a human Caspase-1 (CASP1) gene, or a homolog, variant, fragment or derivative thereof. Accordingly, the RNA may comprise a 3′-UTR derived from a CASP1 gene, wherein said 3′-UTR derived from a CASP1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13828 or 13829 or a fragment or a variant thereof.

COX6B1-derived 3′-UTR: The RNA may comprise a 3′-UTR which is derived from a 3′-UTR of a COX6B1 gene encoding a cytochrome c oxidase subunit 6B1 (COX6B1) protein, or a homolog, variant, fragment or derivative thereof. Such 3′-UTRs preferably comprise or consist of a nucleic acid sequence which is derived from the 3′-UTR of a cytochrome c oxidase subunit 6B1 (COX6B1) gene, preferably from a vertebrate, more preferably a mammalian, most preferably a human cytochrome c oxidase subunit 6B1 (COX6B1) gene, or a homolog, variant, fragment or derivative thereof. Accordingly, the RNA may comprise a 3′-UTR derived from a COX6B1 gene, wherein said 3′-UTR derived from a COX6B1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13830 or 13831 or a fragment or a variant thereof.

GNAS-derived 3′-UTR: The RNA may comprise a 3′-UTR which is derived from a 3′-UTR of a GNAS gene encoding a Guanine nucleotide-binding protein G(s) subunit alpha isoforms short (GNAS) protein, or a homolog, variant, fragment or derivative thereof. Such 3′-UTRs preferably comprise or consist of a nucleic acid sequence which is derived from the 3′-UTR of a Guanine nucleotide-binding protein G(s) subunit alpha isoforms short (GNAS) gene, preferably from a vertebrate, more preferably a mammalian Guanine nucleotide- binding protein G(s) subunit alpha isoforms short (GNAS) gene, or a homolog, variant, fragment or derivative thereof. Accordingly, the RNA may comprise a 3′-UTR derived from a GNAS gene, wherein said 3′-UTR derived from a GNAS gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13832 or 13833 or a fragment or a variant thereof.

NDUFA1-derived 3′-UTR: The RNA may comprise a 3′-UTR which is derived from a 3′-UTR of a gene encoding a NADH dehydrogenase [ubiquinone] 1 alpha sub complex subunit 1 (NDUFA1) protein, or a homolog, variant, fragment or derivative thereof. Such 3′-UTRs preferably comprise or consist of a nucleic acid sequence derived from the 3′-UTR of a NADH dehydrogenase [ubiquinone] 1 alpha sub complex subunit 1 (NDUFA1) gene, preferably from a vertebrate, more preferably a mammalian NADH dehydrogenase [ubiquinone] 1 alpha sub complex subunit 1 (NDUFA1) gene, or a homolog, variant, fragment or derivative thereof. Accordingly, the RNA may comprise a 3′-UTR derived from a NDUFA1 gene, wherein said 3′-UTR derived from a NDUFA1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13834 or 13835 or a fragment or a variant thereof.

RPS9-derived 3′-UTR: The RNA may comprise a 3′-UTR which is derived from a 3′-UTR of a gene encoding a 40S ribosomal protein S9 (RPS9) protein, or a homolog, variant, fragment or derivative thereof. Such 3′-UTRs preferably comprise or consist of a nucleic acid sequence derived from the 3′-UTR of a 40S ribosomal protein S9 (RPS9) gene, preferably from a vertebrate, more preferably a mammalian, most preferably a human 40S ribosomal protein S9 (RPS9) gene, or a homolog, variant, fragment or derivative thereof. Accordingly, the RNA may comprise a 3′-UTR derived from a RPS9 gene, wherein said 3′-UTR derived from a RPS9 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13836 or 13837 or a fragment or a variant thereof.

Further suitable 3′-UTRs: In embodiments, the RNA of the first aspect comprises a 3′-UTR as described in WO2016/107877, the disclosure of WO2016/107877 relating to 3′-UTR sequences herewith incorporated by reference. Suitable 3′-UTRs are SEQ ID NOs: 1 to 24 and SEQ ID NOs: 49 to 318 of WO2016/107877, or fragments or variants of these sequences. Accordingly, the 3′-UTRs of the RNA may comprise or consist of a corresponding RNA sequence of the nucleic acid sequence according SEQ ID NOs: 1 to 24 and SEQ ID NOs: 49 to 318 of WO2016/107877. In other embodiments, the RNA of the first aspect comprises a 3′-UTR as described in WO2017/036580, the disclosure of WO2017/036580 relating to 3′-UTR sequences herewith incorporated by reference. Suitable 3′-UTRs are SEQ ID NOs: 152 to 204 of WO2017/036580, or fragments or variants of these sequences. Accordingly, the 3′-UTR of the RNA may comprise or consist of a corresponding RNA sequence of the nucleic acid sequence according SEQ ID NOs: 152 to 204 of WO2017/036580.

In preferred embodiments, the at least one RNA comprises at least one heterologous 5′-UTR.

Preferably the RNA comprises a 5′-UTR, which may be derivable from a gene that relates to an RNA with enhanced half-life (i.e. that provides a stable RNA).

In preferred embodiments of the first aspect, the RNA comprises at least one heterologous 5′-UTR, wherein the at least one heterologous 5′-UTR comprises a nucleic acid sequence derived from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes. Particularly preferred 5′-UTRs are HSD17B4, NDUFA4 or RPL32.

RPL32-derived 5′-UTR: The RNA may comprise a 5′-UTR derived from a 5′-UTR of a gene encoding a 60S ribosomal protein L32, or a homolog, variant, fragment or derivative thereof, wherein said 5′-UTR preferably lacks the TOP motif. Such 5′-UTRs preferably comprise or consist of a nucleic acid sequence derived from the 5′-UTR of a 60S ribosomal protein L32 (RPL32) gene, preferably from a vertebrate, more preferably a mammalian, most preferably a human 60S ribosomal protein L32 (RPL32) gene, or a homolog, variant, fragment or derivative thereof, wherein the 5′-UTR preferably does not comprise the TOP motif of said gene. Accordingly, the RNA may comprise a 5′-UTR derived from a RPL32 gene, wherein said 5′-UTR derived from a RPL32 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13824 or 13825 or a fragment or a variant thereof.

HSD17B4-derived 5′-UTR: The RNA may comprise a 5′-UTR derived from a 5′-UTR of a gene encoding a 17-beta-hydroxysteroid dehydrogenase 4, or a homolog, variant, fragment or derivative thereof, preferably lacking the TOP motif. Such 5′-UTRs preferably comprise or consist of a nucleic acid sequence derived from the 5′-UTR of a 17-beta-hydroxysteroid dehydrogenase 4 gene, preferably from a vertebrate, more preferably mammalian, most preferably human 17-beta-hydroxysteroid dehydrogenase 4 (HSD17B4) gene, or a homolog, variant, fragment or derivative thereof, wherein preferably the 5′-UTR does not comprise the TOP motif of said gene. Accordingly, the RNA may comprise a 5′-UTR derived from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13804 or 13805 or a fragment or a variant thereof.

ASAH1-derived 5′-UTR: The RNA may comprise a 5′-UTR derived from a 5′-UTR of a gene encoding acid ceramidase (ASAH1), or a homolog, variant, fragment or derivative thereof. Such 5′-UTRs preferably comprise or consist of a nucleic acid sequence derived from the 5′-UTR of an acid ceramidase (ASAH1) gene, preferably from a vertebrate, more preferably mammalian, most preferably human acid ceramidase (ASAH1) gene, or a homolog, variant, fragment or derivative thereof. Accordingly, the RNA may comprise a 5′-UTR derived from a ASAH1 gene, wherein said 5′-UTR derived from a ASAH1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13806 or 13807 or a fragment or a variant thereof.

ATP5A1-derived 5′-UTR: The RNA may comprise a 5′-UTR which is derived from a 5′-UTR of a gene encoding mitochondrial ATP synthase subunit alpha (ATP5A1), or a homolog, variant, fragment or derivative thereof, wherein said 5′-UTR preferably lacks the TOP motif. Such 5′-UTRs preferably comprise or consist of a nucleic acid sequence derived from the 5′-UTR of a mitochondrial ATP synthase subunit alpha (ATP5A1) gene, preferably from a vertebrate, more preferably a mammalian and most preferably a human mitochondrial ATP synthase subunit alpha (ATP5A1) gene, or a homolog, variant, fragment or derivative thereof, wherein the 5′-UTR preferably does not comprise the TOP motif of said gene. Accordingly, the RNA may comprise a 5′-UTR derived from a ATP5A1 gene, wherein said 5′-UTR derived from a ATP5A1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13808 or 13809 or a fragment or a variant thereof.

MP68-derived 5′-UTR: The RNA may comprise a 5′-UTR which is derived from a 5′-UTR of a gene encoding MP68, or a homolog, fragment or variant thereof. Such 5′-UTRs preferably comprise or consist of a nucleic acid sequence derived from the 5′-UTR of a 6.8 kDa mitochondrial proteolipid (MP68) gene, preferably from a vertebrate, more preferably a mammalian 6.8 kDa mitochondrial proteolipid (MP68) gene, or a homolog, variant, fragment or derivative thereof. Accordingly, the RNA may comprise a 5′-UTR derived from a MP68 gene, wherein said 5′-UTR derived from a MP68 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13810 or 13811 or a fragment or a variant thereof.

NDUFA4-derived 5′-UTR: The RNA may comprise a 5′-UTR which is derived from a 5′-UTR of a gene encoding a Cytochrome c oxidase subunit (NDUFA4), or a homolog, fragment or variant thereof. Such 5′-UTRs preferably comprise or consist of a nucleic acid sequence derived from the 5′-UTR of a Cytochrome c oxidase subunit (NDUFA4) gene, preferably from a vertebrate, more preferably a mammalian Cytochrome c oxidase subunit (NDUFA4) gene, or a homolog, variant, fragment or derivative thereof. Accordingly, the RNA may comprise a 5′-UTR derived from a NDUFA4 gene, wherein said 5′-UTR derived from a NDUFA4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13812 or 13813 or a fragment or a variant thereof.

NOSIP-derived 5′-UTR: The RNA may comprise a 5′-UTR which is derived from a 5′-UTR of a gene encoding a Nitric oxide synthase-interacting (NOSIP) protein, or a homolog, variant, fragment or derivative thereof. Such 5′-UTRs preferably comprise or consist of a nucleic acid sequence derived from the 5′-UTR of a Nitric oxide synthase-interacting protein (NOSIP) gene, preferably from a vertebrate, more preferably a mammalian, most preferably a human Nitric oxide synthase-interacting protein (NOSIP) gene, or a homolog, variant, fragment or derivative thereof. Accordingly, the RNA may comprise a 5′-UTR derived from a NOSIP gene, wherein said 5′-UTR derived from a NOSIP gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13814 or 13815 or a fragment or a variant thereof.

RPL31-derived 5′-UTR: The RNA may comprise a 5′-UTR which is derived from a 5′-UTR of a gene encoding a 60S ribosomal protein L31, ora homolog, variant, fragment or derivative thereof, wherein said 5′-UTR preferably lacks the TOP motif. Such 5′-UTR preferably comprise or consist of a nucleic acid sequence derived from the 5′-UTR of a 60S ribosomal protein L31 (RPL31) gene, preferably from a vertebrate, more preferably a mammalian 60S ribosomal protein L31 (RPL31) gene, or a homolog, variant, fragment or derivative thereof, wherein the 5′-UTR preferably does not comprise the TOP motif of said gene. Accordingly, the RNA may comprise a 5′-UTR derived from a RPL31 gene, wherein said 5′-UTR derived from a RPL31 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13816 or 13817 or a fragment or a variant thereof.

SLC7A3-derived 5′-UTR: The RNA may comprise a 5′-UTR which is derived from a 5′-UTR of a gene encoding a cationic amino acid transporter 3 (solute carrier family 7 member 3, SLC7A3) protein, or a homolog, variant, fragment or derivative thereof. Such 5′-UTRs preferably comprise or consist of a nucleic acid sequence derived from the 5′-UTR of a cationic amino acid transporter 3 (SLC7A3) gene, preferably from a vertebrate, more preferably a mammalian cationic amino acid transporter 3 (SLC7A3) gene, or a homolog, variant, fragment or derivative thereof. Accordingly, the RNA may comprise a 5′-UTR derived from a SLC7A3 gene, wherein said 5′-UTR derived from a SLC7A3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13818 or 13819 or a fragment or a variant thereof.

TUBB4B-derived 5′-UTR: The RNA may comprise a 5′-UTR which is derived from a 5′-UTR of a gene encoding a tubulin beta-4B chain (TUBB4B) protein, or a homolog, variant, fragment or derivative thereof. Such 5′-UTRs preferably comprise or consist of a nucleic acid sequence derived from the 5′-UTR of a tubulin beta-4B chain (TUBB4B) gene, preferably from a vertebrate, more preferably a mammalian and most preferably a human tubulin beta-4B chain (TUBB4B) gene, or a homolog, variant, fragment or derivative thereof. Accordingly, the RNA may comprise a 5′-UTR derived from a TUBB4B gene, wherein said 5′-UTR derived from a TUBB4B gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13821 or 13821 or a fragment or a variant thereof.

UBQLN2-derived 5′-UTR: The RNA may comprise a 5′-UTR which is derived from a 5′-UTR of a gene encoding an ubiquilin-2 (UBQLN2) protein, or a homolog, variant, fragment or derivative thereof. Such 5′-UTRs preferably comprise or consist of a nucleic acid sequence derived from the 5′-UTR of an ubiquilin-2 (UBQLN2) gene, preferably from a vertebrate, more preferably a mammalian ubiquilin-2 (UBQLN2) gene, or a homolog, variant, fragment or derivative thereof. Accordingly, the RNA may comprise a 5′-UTR derived from a UBQLN2 gene, wherein said 5′-UTR derived from a UBQLN2 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13822 or 13823 or a fragment or a variant thereof.

Further suitable 5′-UTRs: In embodiments, the RNA of the first aspect comprises a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In this context, it is preferred that the 5′-UTR of the RNA comprises or consists of a corresponding RNA sequence of the nucleic acid sequence according SEQ ID NOs: 1-1363, SEQ ID NO: 1395, SEQ ID NO: 1421 and SEQ ID NO: 1422 of WO2013/143700. In other embodiments, the RNA of the first aspect comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25 to 30 and SEQ ID NOs: 319 to 382 of WO2016/107877, or fragments or variants of these sequences. In this context, it is particularly preferred that the 5′-UTR of the RNA comprises or consists of a corresponding RNA sequence of the nucleic acid sequence according SEQ ID NOs: 25 to 30 and SEQ ID NOs: 319 to 382 of WO2016/107877. In other embodiments, the RNA of the first aspect comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1 to 151 of WO2017/036580, or fragments or variants of these sequences. In this context, it is particularly preferred that the 5′-UTR of the RNA comprises or consists of a corresponding RNA sequence of the nucleic acid sequence according to SEQ ID NOs: 1 to 151 of WO2017/036580.

The inventors observed that certain combinations of 5′-UTR and/or 3′-UTR as described herein may increase the expression of the at least one coding sequence (encoding at least one antigenic peptide or protein derived from a LASV protein). Said increase in LASV protein expression may be particularly pronounced in the dermis (after intradermal application), the epidermis (after epidermal application) or, most advantageously, in the muscle (after intramuscular application).

Accordingly it is preferred that the at least one heterologous 5′-UTR as defined herein and the at least one heterologous 3′-UTR as defined herein act synergistically to increase production (that is translation) of antigenic peptide or protein from the RNA of the first aspect. These advantageous combinations of 5′-UTR and 3′-UTR are specified in the following. Each of the abbreviation introduced below, namely “a-1”, “a-2”, “a-3”, “a-4”, “a-5”, “b-1”, “b-2”, “b-3”, “b-4”, “b-5”, “c-1”, “c-2”, “c-3”, “c-4”, “c-5”, “d-1”, “d-2”, “d-3”, “d-4”, “d-5”, “e-1”, “e-2”, “e-3”, “e-4”, “e-5”, “e-6”, “f-1”, 1-2″, 1-3″, 1-4″,1-5″, “g-1”, “g-2”, “g-3”, “g-4”, “g-5”, “h-1”, “h-2”, “h-3”, “h-4”, “h-5”, 1-1″, “i-2”, “i-3”, are used throughout the specification of the present invention and represent one advantageous combination of 5′-UTR and/or 3′UTR.

Accordingly, in a preferred embodiment of the first aspect, the RNA comprises at least one coding sequence encoding at least one antigenic peptide or protein derived from a Lassa virus (LASV) protein or a fragment or variant thereof, wherein said coding sequence is operably linked to 5′-UTR and/or 3′-UTR, comprising
a-1. at least one 5′-UTR derived from a 5′-UTR of a HSD17B4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-2. at least one 5′-UTR derived from a 5′-UTR of a NDUFA4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
b-4. at least one 5′-UTR derived from a 5′-UTR of a HSD17B4 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
a-3. at least one 5′-UTR derived from a 5′-UTR of a SLC7A3 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-4. at least one 5′-UTR from a 5′-UTR of a NOSIP gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-5. at least one 5′-UTR derived from a 5′-UTR of a MP68 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
b-1. at least one 5′-UTR derived from a 5′-UTR of a UBQLN2 gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
b-2. at least one 5′-UTR derived from a 5′-UTR of a ASAH1 gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
b-3. at least one 5′-UTR derived from a 5′-UTR of a HSD17B4 gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
b-5. at least one 5′-UTR derived from a 5′-UTR of a NOSIP gene, or from a corresponding RNA sequence, homolog, fragment or variant thereof and at least one 3′-UTR derived from a 3′-UTR of a COX6B1 gene, or from a corresponding RNA sequence, homolog, fragment or variant thereof; or
c-1. at least one 5′-UTR derived from a 5′-UTR of a NDUFA4 gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
c-2. at least one 5′-UTR derived from a 5′-UTR of a NOSIP gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
c-3. at least one 5′-UTR derived from a 5′-UTR of a NDUFA4 gene, or from a corresponding RNA sequence, homolog, fragment or variant thereof and at least one 3′-UTR derived from a 3′-UTR of a COX6B1 gene, or from a corresponding RNA sequence, homolog, fragment or variant thereof; or
c-4. at least one 5′-UTR derived from a 5′-UTR of a NDUFA4 gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
c-5. at least one 5′-UTR derived from a 5′-UTR of a ATP5A1 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
d-1. at least one 5′-UTR derived from a 5′-UTR of a RPL31 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
d-2. at least one 5′-UTR derived from a 5′-UTR of a ATP5A1 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
d-3. at least one 5′-UTR derived from a 5′-UTR of a SLC7A3 gene, or from a corresponding RNA sequence, homolog, fragment or variant thereof and at least one 3′-UTR derived from a 3′-UTR of a GNAS gene, or from a corresponding RNA sequence, homolog, fragment or variant thereof; or
d-4. at least one 5′-UTR derived from a 5′-UTR of a HSD17B4 gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
d-5. at least one 5′-UTR derived from a 5′-UTR of a SLC7A3 gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
e-1. at least one 5′-UTR derived from a 5′-UTR of a TUBB4B gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
e-2. at least one 5′-UTR derived from a 5′-UTR of a RPL31 gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
e-3. at least one 5′-UTR derived from a 5′-UTR of a MP68 gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
e-4. at least one 5′-UTR derived from a 5′-UTR of a NOSIP gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
e-5. at least one 5′-UTR derived from a 5′-UTR of a ATP5A1 gene, or from a corresponding RNA sequence, homolog, fragment or variant thereof and at least one 3′-UTR derived from a 3′-UTR of a RPS9 gene, or from a corresponding RNA sequence, homolog, fragment or variant thereof; or
e-6. at least one 5′-UTR derived from a 5′-UTR of a ATP5A1 gene, or from a corresponding RNA sequence, homolog, fragment or variant thereof and at least one 3′-UTR derived from a 3′-UTR of a COX6B1 gene, or from a corresponding RNA sequence, homolog, fragment or variant thereof; or
f-1. at least one 5′-UTR derived from a 5′-UTR of a ATP5A1 gene, or from a corresponding RNA sequence, homolog, fragment or variant thereof and at least one 3′-UTR derived from a 3′-UTR of a GNAS gene, or from a corresponding RNA sequence, homolog, fragment or variant thereof; or
f-2. at least one 5′-UTR derived from a 5′-UTR of a ATP5A1 gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
f-3. at least one 5′-UTR derived from a 5′-UTR of a HSD17B4 gene, or from a corresponding RNA sequence, homolog, fragment or variant thereof and at least one 3′-UTR derived from a 3′-UTR of a COX6B1 gene, or from a corresponding RNA sequence, homolog, fragment or variant thereof; or
f-4. at least one 5′-UTR derived from a 5′-UTR of a HSD17B4 gene, or from a corresponding RNA sequence, homolog, fragment or variant thereof and at least one 3′-UTR derived from a 3′-UTR of a GNAS gene, or from a corresponding RNA sequence, homolog, fragment or variant thereof; or
f-5. at least one 5′-UTR derived from a 5′-UTR of a MP68 gene, or from a corresponding RNA sequence, homolog, fragment or variant thereof and at least one 3′-UTR derived from a 3′-UTRof a COX6B1 gene, or from a corresponding RNA sequence, homolog, fragment or variant thereof; or
g-1. at least one 5′-UTR derived from a 5′-UTR of a MP68 gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
g-2. at least one 5′-UTR derived from a 5′-UTR of a NDUFA4 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
g-3. at least one 5′-UTR derived from a 5′-UTR of a NDUFA4 gene, or from a corresponding RNA sequence, homolog, fragment or variant thereof and at least one 3′-UTR derived from a 3′-UTR of a GNAS gene, or from a corresponding RNA sequence, homolog, fragment or variant thereof; or
g-4. at least one 5′-UTR derived from a 5′-UTR of a NOSIP gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
g-5. at least one 5′-UTR derived from a 5′-UTR of a RPL31 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
h-1. at least one 5′-UTR derived from a 5′-UTR of a RPL31 gene, or from a corresponding RNA sequence, homolog, fragment or variant thereof and at least one 3′-UTR derived from a 3′-UTR of a COX6B1 gene, or from a corresponding RNA sequence, homolog, fragment or variant thereof; or
h-2. at least one 5′-UTR derived from a 5′-UTR of a RPL31 gene, or from a corresponding RNA sequence, homolog, fragment or variant thereof and at least one 3′-UTR derived from a 3′-UTR of a GNAS gene, or from a corresponding RNA sequence, homolog, fragment or variant thereof; or
h-3. at least one 5′-UTR derived from a 5′-UTR of a RPL31 gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
h-4. at least one 5′-UTR derived from a 5′-UTR of a SLC7A3 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
h-5. at least one 5′-UTR derived from a 5′-UTR of a SLC7A3 gene, or from a corresponding RNA sequence, homolog, fragment or variant thereof and at least one 3′-UTR derived from a 3′-UTR of a COX6B1 gene, or from a corresponding RNA sequence, homolog, fragment or variant thereof; or
i-1. at least one 5′-UTR derived from a 5′-UTR of a SLC7A3 gene, or from a corresponding RNA sequence, homolog, fragment or variant thereof and at least one 3′-UTR derived from a 3′-UTR of a RPS9 gene, or from a corresponding RNA sequence, homolog, fragment or variant thereof.
i-2. at least one 5′-UTR derived from a 5′-UTR of a RPL32 gene, or from a corresponding RNA sequence, homolog, fragment or variant thereof and at least one 3′-UTR derived from a 3′-UTR of a ALB7 gene, or from a corresponding RNA sequence, homolog, fragment or variant thereof.
i-3. at least one 3′-UTR derived from a 3′-UTR of a alpha-globin gene gene, or from a corresponding RNA sequence, homolog, fragment or variant thereof.

Suitably, the RNA comprises at least one coding sequence encoding at least one antigenic peptide or protein derived from a LASV as specified herein operably linked to a 3′-UTR and/or a 5′-UTR selected from a-1 (HSD17B4/PSMB3), a-2 (NDUFA4/PSMB3), a-3 (SLC7A3/PSMB3), a-4 (NOSIP/PSMB3), a-5 (MP68/PSMB3), b-1 (UBQLN2/RPS9), b-2 (ASAH1/RPS9), b-3 (HSD17B4/RPS9), b-4 (HSD17B4/CASP1), b-5 (NOSIP/COX6B1), c-1 (NDUFA4/RPS9), c-2 (NOSIP/NDUFA1), c-3 (NDUFA4/COX6B1), c-4 (NDUFA4/NDUFA1), c-5 (ATP5A1/PSMB3), d-1 (RpI31/PSMB3), d-2 (ATP5A1/CASP1), d-3 (SLC7A3/GNAS), d-4 (HSD17B4/NDUFA1), d-5 (S1c7a3/Ndufa1), e-1 (TUBB4B/RPS9), e-2 (RPL31/RPS9), e-3 (MP68/RPS9), e-4 (NOSIP/RPS9), e-5 (ATP5A1/RPS9), e-6 (ATP5A1/COX6B1), f-1 (ATP5A1/GNAS), f-2 (ATP5A1/NDUFA1), f-3 (HSD17B4/COX6B1), f-4 (HSD17B4/GNAS), f-5 (MP68/COX6B1), g-1 (MP68/NDUFA1), g-2 (NDUFA4/CASP1), g-3 (NDUFA4/GNAS), g-4 (NOSIP/CASP1), g-5 (RPL31/CASP1), h-1 (RPL31/COX6B1), h-2 (RPL31/GNAS), h-3 (RPL31/NDUFA1), h-4 (S1c7a3/CASP1), h-5 (SLC7A3/COX6B1), i-1 (SLC7A3/RPS9), i-2 (RPL32/ALB7), or i-3 (α-globin gene).

In particularly preferred embodiments of the first aspect, the RNA comprise UTR elements according to a-1 (HSD17B4/PSMB3), a-2 (NDUFA4/PSMB3), b-4 (HSD17B4/ CASP1), i-2 (RPL32/ALB7), or i-3 (alpha-globin, “muag”).

The RNA comprises at least one coding sequence operably linked to said 3′-UTR and/or 5′-UTR, wherein said coding sequence encodes at least one antigenic peptide or protein derived from a LASV protein, or a fragment or variant thereof. Advantageously in the context of the invention, the RNA of the first aspect is suitable for vaccination.

As used herein, the term “Lassa virus” or “Lassa mammarenavirus” or the corresponding abbreviation “LASV” is not limited to a particular virus strain, variant, serotype, clade member, lineage, or isolate, etc., and therefore comprises any LASV of any origin (NCBI:txid11620). LASV can be grouped into several different clades or lineages (I, II, III, IV, V, VI, etc.). The term clade is preferably used synonymously with the term lineages. Suitable LASV clade members are provided in List 1-6.

LASV is a member of the arenavirus family, which includes more than 30 known pathogens that exist on all populated continents on Earth. The Old World family of arenaviruses contains LASV; lymphocytic choriomeningitis virus (LCMV), which causes febrile illness, neurological disease, and birth defects with a 2 to 5% seroprevalence in North America and Europe; and the 80% lethal hemorrhagic fever virus Lujo (LUJV), which emerged in Southern Africa in 2008. Arenaviruses in the New World category include Machupo virus (MACV) and Junin virus (JUNV), the causative agents of Bolivian and Argentinian hemorrhagic fever, respectively, as well as numerous other agents such as Sabia and Guanarito (Hastie, Kathryn M., et al. “Structural basis for antibody-mediated neutralization of Lassa virus.” Science 356.6341 (2017): 923-928). The invention is not only limited to “Lassa virus” or “Lassa mammarenavirus”, further members of the arenavirus family, known as pathogens, are also included, suitably selected from LCMV (WE-HPI), LUJV, JUNV (MN-2) and MACV (Carvallo).

List 1: LASV Clade I or LASV Lineage I Members:

In embodiments, the LASV from which the antigenic peptide or protein is derived from is a clade I member, wherein said clade I member may preferably be selected from a list comprising LP, Pinneo-NIG-1969, Acar 3080; accordingly, when reference is made to “LASV clade I” or “LASV lineage I”, said LASV clade I member may be preferably selected from said list. Preferred LASV clade I members in the context of the invention are LP, or Pinneo-NIG-1969.

List 2: LASV Clade II or LASV Lineage II Members:

In embodiments, the LASV from which the antigenic peptide or protein is derived from is a clade II member, wherein said clade II member may preferably be selected from a list comprising 803213, ISTH0009-NIG-2011, ISTH0012-NIG-2011, ISTH0047-NIG-2011, ISTH0073-NIG-2011, ISTH0230-NIG-2011, ISTH0531-NIG-2011, ISTH0595-NIG-2011, ISTH0964-NIG-2011, ISTH1003-NIG-2011, ISTH1038-NIG-2011, ISTH1048-NIG-2011, ISTH1058-NIG-2011, ISTH1064-NIG-2011, ISTH1069-NIG-2011, ISTH1096-NIG-2012, ISTH1107-NIG-2012, ISTH1111-NIG-2011, ISTH1121-NIG-2012, ISTH1129-NIG-2012, ISTH1137-NIG-2011, ISTH2010-NIG-2012, ISTH2016-NIG-2012, ISTH2020-NIG-2012, ISTH2025-NIG-2012, ISTH2031-NIG-2012, ISTH2037-NIG-2012, ISTH2042-NIG-2012, ISTH2046-NIG-2012, ISTH2050-NIG-2012, ISTH2057-NIG-2012, ISTH2061-NIG-2012, ISTH2064-NIG-2012, ISTH2065-NIG-2012, ISTH2066-NIG-2012, ISTH2069-NIG-2012, ISTH2094-NIG-2012, ISTH2129-NIG-2012, ISTH2217-NIG-2012, ISTH2271-NIG-2012, ISTH2304-NIG-2012, ISTH2312-NIG-2012, ISTH2316-NIG-2012, ISTH2334-NIG-2012, ISTH2358-NIG-2012, ISTH2376-NIG-2012, Lassa virus/H.sapiens-wt/NGA/2015/ISTH_0543, Lassa virus/H.sapiens-wt/NGA/2015/ISTH_0547, Lassa virus/H.sapiens-wt/NGA/2015/ISTH_0574, Lassa virus/H.sapiens-wt/NGA/2015/ISTH _0681, Lassa virus/H.sapiens-wt/NGA/2015/ISTH_0682, Lassa virus/H.sapiens-wt/NGA/2015/ISTH_0687, Lassa virus/H.sapiens-wt/NGA/2015/ISTH_0736, Lassa virus/H.sapiens-wt/NGA/2015/ISTH_0789, Lassa virus/H.sapiens-wt/NGA/2015/ISTH_0821, Lassa virus/H.sapiens-wt/NGA/2015/ISTH_0831, Lassa virus/H.sapiens-wt/NGA/2016/ISTH _0009, Lassa virus/H.sapiens-wt/NGA/2016/ISTH_0017, Lassa virus/H.sapiens-wt/NGA/2016/ISTH_0035, Lassa virus/H.sapiens-wt/NGA/2016/ISTH_0089, Lassa virus/H.sapiens-wt/NGA/2016/ISTH_0104, Lassa virus/H.sapiens-wt/NGA/2016/ISTH_0133, Lassa virus/H.sapiens-wt/NGA/2016/ISTH_0141, Lassa virus/H.sapiens-wt/NGA/2016/ISTH _0146, Lassa virus/H.sapiens-wt/NGA/2016/ISTH_0176, Lassa virus/H.sapiens-wt/NGA/2016/ISTH_0187, Lassa virus/H.sapiens-wt/NGA/2016/ISTH_0199, Lassa virus/H.sapiens-wt/NGA/2016/ISTH_0419, Lassa virus/H.sapiens-wt/NGA/2016/ISTH_0447, Lassa virus/H.sapiens-wt/NGA/2016/ISTH_0543, Lassa virus/H.sapiens-wt/NGA/2016/ISTH _0565, Lassa virus/H.sapiens-wt/NGA/2016/ISTH_0572, Lassa virus/H.sapiens-wt/NGA/2016/ISTH_0610, Lassa virus/H.sapiens-wt/NGA/2016/ISTH_0621, Lassa virus/H.sapiens-wt/NGA/2016/ISTH_0701, Lassa virus/H.sapiens-wt/NGA/2016/ISTH_0702, Lassa virus/H.sapiens-wt/NGA/2016/ISTH_0703, Lassa virus/H.sapiens-wt/NGA/2016/ISTH _0717, Lassa virus/H.sapiens-wt/NGA/2016/ISTH_0753, Lassa virus/H.sapiens-wt/NGA/2016/ISTH_0759, Lassa virus/H.sapiens-wt/NGA/2016/ISTH_0777, Lassa virus/H.sapiens-wt/NGA/2016/ISTH_0779, Lassa virus/H.sapiens-wt/NGA/2016/ISTH_0917, Lassa virus/H.sapiens-wt/NGA/2016/ISTH_0919, Lassa virus/H.sapiens-wt/NGA/2016/ISTH _1006, Lassa virus/H.sapiens-wt/NGA/2016/ISTH_1026, Lassa virus/H.sapiens-wt/NGA/2016/ISTH_1069, Lassa virus/H.sapiens-wt/NGA/2016/ISTH_1070, Lassa virus/H.sapiens-wt/NGA/2016/ISTH_1123, Lassa virus/H.sapiens-wt/NGA/2016/ISTH_1150, Lassa virus/H.sapiens-wt/NGA/2016/ISTH_1174, Lassa virus/H.sapiens-wt/NGA/2016/ISTH _1207, Lassa virus/H.sapiens-wt/NGA/2016/ISTH_1250, Lassa virus/H.sapiens-wt/NGA/2016/ISTH_1306, Lassa virus/H.sapiens-wt/NGA/2016/ISTH_1311, Lassa virus/H.sapiens-wt/NGA/2016/ISTH_1398, Lassa virus/H.sapiens-wt/NGA/2016/ISTH_1423, Lassa virus/H.sapiens-wt/NGA/2016/ISTH_1462, Lassa virus/H.sapiens-wt/NGA/2016/ ISTH_1472, Lassa virus/H.sapiens-wt/NGA/2016/ISTH_1474, Lassa virus/H.sapiens-wt/NGA/2016/ISTH_1496, Lassa virus/H.sapiens-wt/NGA/2016/ISTH_1541, Lassa virus/H.sapiens-wt/NGA/2016/ISTH_1546, Lassa virus/H.sapiens-wt/NGA/2016/ISTH_1568, Lassa virus/H.sapiens-wt/NGA/2016/ISTH_1591, Lassa virus/H.sapiens-wt/NGA/2016/ ISTH_1604, Lassa virus/H.sapiens-wt/NGA/2016/ISTH_1609, Lassa virus/H.sapiens-wt/NGA/2016/ISTH_1610, Lassa virus/H.sapiens-wt/NGA/2018/ISTH_0026, Lassa virus/H.sapiens-wt/NGA/2018/ISTH_0097, Lassa virus/H.sapiens-wt/NGA/2018/ISTH_0541, Lassa virus/H.sapiens-wt/NGA/2018/ISTH_0664, Lassa virus/H.sapiens-wt/NGA/2018/ ISTH_0959, Lassa virus/H.sapiens-wt/NGA/2018/ISTH_1024, Lassa virus/H.sapiens-wt/NGA/2018/ISTH_1079, Lassa virus/H.sapiens-wt/NGA/2018/ISTH_1177, Lassa virus/H.sapiens-wt/NGA/2018/ISTH_1375, Lassa virus/H.sapiens-wt/NGA/2018/ISTH_1381, Lassa virus/H.sapiens-wt/NGA/2018/ISTH1643, LASV003-NIG-2008, LASV006-NIG-2008, LASV035-NIG-2009, LASV042-NIG-2009, LASV045-NIG-2009, LASV046-NIG-2009, LASV049-NIG-2009, LASV052-NIG-2008, LASV056-NIG-2008, LASV058-NIG-2008, LASV063-NIG-2009, LASV1000-NIG-2009, LASV1008-NIG-2009, LASV1011-NIG-2009, LASV1015-NIG-2009, LASV1016-NIG-2009, LASV221-NIG-2010, LASV224-NIG-2010, LASV225-NIG-2010, LASV229-NIG-2010, LASV237-NIG-2010, LASV239-NIG-2010, LASV241-NIG-2011, LASV242-NIG-2010, LASV245-NIG-2011, LASV246-NIG-2010, LASV250-NIG-2011, LASV251-NIG-2010, LASV253-NIG-2011, LASV254-NIG-2011, LASV256-NIG-2010, LASV263-NIG-2011, LASV267-NIG-2010, LASV271-NIG-2010, LASV274-NIG-2010, LASV711-NIG-2009, LASV716-NIG-2009, LASV719-NIG-2009, LASV736-NIG-2009, LASV737-NIG-2009, LASV738-NIG-2009, LASV746-NIG-2009, LASV966-NIG-2009, LASV967-NIG-2009, LASV969-NIG-2009, LASV971-NIG-2009, LASV975-NIG-2009, LASV976-NIG-2009, LASV977-NIG-2009, LASV978-NIG-2009, LASV979-NIG-2009, LASV981-NIG-2009, LASV982-NIG-2009, LASV988-NIG-2009, LASV989-NIG-2009, LASV990-NIG-2009, LASV991-NIG-2009, LASV992-NIG-2009, LASV993-NIG-2009, Nig08-04, Nig08-A37, Nig08-A41, Nig08-A47, or NIGERIA IKEJI; accordingly, when reference is made to “LASV clade II” or “LASV lineage II”, said LASV clade II member may be preferably selected from said list. Preferred LASV clade II members in the context of the invention are 803213, ISTH0009-NIG-2011, ISTH2010-NIG-2012, Lassa virus/H.sapiens-wt/NGA/2015/ISTH_0543, Lassa virus/H.sapiens-wt/NGA/2016/ISTH_0009, Lassa virus/H.sapiens-wt/NGA/2016/ISTH_0017, Lassa virus/H.sapiens-wt/NGA/2018/ISTH_0959, LASV003-NIG-2008, LASV035-NIG-2009, LASV1000-NIG-2009, LASV221-NIG-2010, LASV237-NIG-2010, LASV253-NIG-2011, LASV254-NIG-2011, or Nig08-04.

List 3: LASV Clade III or LASV Lineage III Members:

In embodiments, the LASV from which the antigenic peptide or protein is derived from is a clade III member, wherein said clade III member may preferably be selected from a list comprising GA391, CSF, ISTH2121-NIG-2012, Nig08-A18, Nig08-A19, ONM-299, ONM-314, ONM-700, or Weller; accordingly, when reference is made to “LASV clade III” or “LASV lineage III”, said LASV clade III member may be preferably selected from said list. Preferred LASV clade III members in the context of the invention are GA391, or Nig08-A19.

List 4: LASV Clade IV or LASV Lineage IV Members:

In embodiments, the LASV from which the antigenic peptide or protein is derived from is a clade IV member, wherein said clade IV member may preferably be selected from a list comprising Josiah, BA366, G1180-SLE-2010, G1190-SLE-2010, G1200-LIB-2010, G1442-SLE-2011, G1529-SLE-2011, G1618-SLE-2011, G1646-SLE-2011, G1647-SLE-2011, G1727-SLE-2011, G1774-SLE-2011, G1792-SLE-2011, G1897-SLE-2011, G1932-SLE-2011, G1959-SLE-2011, G1960-SLE-2011, G2141-SLE-2011, G2147-SLE-2011, G2165-SLE-2011, G2184-SLE-2011, G2197-SLE-2011, G2222-SLE-2011, G2230-SLE-2012, G2259-SLE-2012, G2263-SLE-2012, G2280-SLE-2012, G2295-SLE-2012, G2315-SLE-2012, G2318-SLE-2012, G2363-SLE-2012, G2384-SLE-2012, G2392-SLE-2012, G2405-SLE-2012, G2427-SLE-2012, G2431-SLE-2012, G2511-SLE-2012, G2554-SLE-2012, G2557-SLE-2012, G2565-SLE-2012, G2587-SLE-2012, G2612-SLE-2012, G2615-SLE-2012, G2723-SLE-2012, G2789-SLE-2012, G2868-SLE-2012, G2903-SLE-2012, G2906-SLE-2012, G2934-SLE-2012, G2944-SLE-2012, G3010-SLE-2013, G3016-SLE-2013, G3034-SLE-2013, G3076-SLE-2013, G3101-SLE-2013, G3106-SLE-2013, G3110-SLE-2013, G3148-SLE-2013, G3151-SLE-2013, G3157-SLE-2013, G3170-SLE-2013, G3206-SLE-2013, G3222-SLE-2013, G3229-SLE-2013, G3234-SLE-2013, G3248-SLE-2013, G3253-SLE-2013, G3278-SLE-2013, G3283-SLE-2013, G502-SLE-2009, G503-SLE-2009, G610-SLE-2009, G636-SLE-2009, G676-SLE-2009, G692-SLE-2009, G693-SLE-2009, G733-SLE-2010, G771-SLE-2010, G806-SLE-2010, G808-SLE-2010, Guinea Faranah, GUINEA Z-185a, IGS-1, IGS-10, IGS-11, IGS-12, IGS-13, IGS-14, IGS-16, IGS-17, IGS-18, IGS-19, IGS-2, IGS-20, IGS-21, IGS-22, IGS-23, IGS-24, IGS-25, IGS-26, IGS-27, IGS-3, IGS-4, IGS-7, IGS-9, IRF0172, IRF0185, IRF0193, IRF0194, IRF0201, IRF0205, LM032-SLE-2010, LM222-SLE-2010, LM395-SLE-2009, LM765-SLE-2012, LM771-SLE-2012, LM774-SLE-2012, LM776-SLE-2012, LM778-SLE-2012, LM779-SLE-2012, Macenta, Mad39, Mad41, Mad62, Mad63, Mad69, Mad83, ML29, NL, recombinant Josiah, Z0947-SLE-2011, Z0948-SLE-2011, or Z148; accordingly, when reference is made to “LASV clade IV” or “LASV lineage IV”, said LASV clade IV member may be preferably selected from said list. Preferred LASV clade IV members in the context of the invention are Josiah, G1180-SLE-2010, G1442-SLE-2011, G3010-SLE-2013, G502-SLE-2009, G733-SLE-2010, MOPV/LASV reassortant IGS-1, LM765-SLE-2012, LM776-SLE-2012, Mad39, or Z0947-SLE-2011.

List 5: LASV Clade V or LASV Lineage V Members:

In embodiments, the LASV from which the antigenic peptide or protein is derived from is a clade V member, wherein said clade V member may preferably be selected from a list comprising AV, Bamba-R114, Komina-R16, Ouoma-R123, Soromba-R, or Soromba-R30; accordingly, when reference is made to “LASV clade V” or “LASV lineage V”, said LASV clade V member may be preferably selected from said list. A preferred LASV clade V member in the context of the invention is AV.

List 6: LASV Clade VI or LASV Lineage VI Members:

In embodiments, the LASV from which the antigenic peptide or protein is derived from is a clade VI member, wherein said clade VI member may preferably be selected from a list comprising Togo/2016/7082, Alzey, Lassa/H.sapiens-tc/TGO/2016/812939, or Lassa/H.sapiens-wt/TGO/2016/201600568; accordingly, when reference is made to “LASV clade VI” or “LASV lineage VI”, said LASV clade VI member may be preferably selected from said list. A preferred LASV clade VI member in the context of the invention is Togo/2016/7082.

Accordingly, the RNA of the first aspect comprises at least one coding sequence encoding at least one antigenic peptide or protein derived from a Lassa virus (LASV) protein or a fragment or variant thereof, wherein said coding sequence is operably linked to a 5′-UTR and/or 3′-UTR, wherein suitably
said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13804 or 13805 or a fragment or a variant thereof;
said 5′-UTR derived from a ASAH1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13806 or 13807 or a fragment or a variant thereof;
said 5′-UTR derived from a ATP5A1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13808 or 13809 or a fragment or a variant thereof;
said 5′-UTR derived from a MP68 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13810 or 13811 or a fragment or a variant thereof;
said 5′-UTR derived from a NDUFA4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13812 or 13813 or a fragment or a variant thereof;
said 5′-UTR derived from a NOSIP gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13814 or 13815 or a fragment or a variant thereof;
said 5′-UTR derived from a RPL31 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13816 or 13817 or a fragment or a variant thereof;
said 5′-UTR derived from a RPL32 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13824 or 13825 or a fragment or a variant thereof;
said 5′-UTR derived from a SLC7A3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13818 or 13819 or a fragment or a variant thereof;
said 5′-UTR derived from a TUBB4B gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13820 or 13821 or a fragment or a variant thereof;
said 5′-UTR derived from a UBQLN2 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13822 or 13823 or a fragment or a variant thereof;
said 3′-UTR derived from a PSMB3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13826 or 13827 or a fragment or a variant thereof;
said 3′-UTR derived from a CASP1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13828 or 13829 or a fragment or a variant thereof;
said 3′-UTR derived from a COX6B1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13830 or 13831 or a fragment or a variant thereof;
said 3′-UTR derived from a GNAS gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13832 or 13833 or a fragment or a variant thereof;
said 3′-UTR derived from a NDUFA1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13834 or 13835 or a fragment or a variant thereof;
said 3′-UTR derived from a RPS9 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13836 or 13837 or a fragment or a variant thereof;
said 3′-UTR derived from a ALB7 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13838 or 13839 or a fragment or a variant thereof;
said 3′-UTR derived from a alpha-globin gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to EQ ID NO: 13840 or 13841 or a fragment or a variant thereof.

In preferred embodiments, the at least one antigenic peptide or protein is derived from glycoprotein precursor (GPC), a prefusion-stabilized GPC, nucleoprotein (NP), zinc-binding matrix protein (Z), or a variant, fragment, or combination thereof. In preferred embodiments, GPC, prefusion-stabilized GPC, NP, and Z are full-length proteins.

The term “full-length protein” or as used herein typically refers to a protein that substantially comprises the entire amino acid sequence of the naturally occurring (wild type) protein. Accordingly, in a preferred embodiment, the at least one coding sequence of the RNA of the first aspect encodes a full-length antigenic protein derived from LASV or a variant of said protein.

As the sole antigen on the viral surface, the LASV glycoprotein complex (GPC) is the primary target of protective humoral immune responses and a focus for vaccine design efforts. The mature GPC is a trimer of heterotrimers, with each heterotrimer composed of the non-covalently associated myristoylated stable signal peptide (SSP), GP1 and GP2 subunits. SSP is required for proper processing of GPC and is retained as part of the complex. GP1 is responsible for receptor binding and determines tropism, while GP2 mediates fusion of the virion with a host cell membrane wherein GP2 undergoes an acid pH-driven, conformational change from a metastable, prefusion structure to a more stable, postfusion structure (see e.g. Hastie, Kathryn M., and Erica Ollmann Saphire. “Lassa virus glycoprotein: stopping a moving target.” Current opinion in virology 31 (2018): 52-58). In preferred embodiments, the coding RNA of the invention encodes at least one antigenic protein that is or is derived from GPC or a fragment or variant thereof.

In embodiments, the coding RNA of the invention encodes at least one antigenic protein that is or is derived from GPC wherein the GPC is a full-length GPC protein having typically an amino acid sequence comprising or consisting of amino acid 1 to amino acid 491 (for e.g. most of the LASV clade/lineages IV members) or of amino acid 1 to amino acid 490 (for e.g. most of LASV clade/lineage I, II, Ill members). In other embodiments, the coding RNA of the invention encodes at least one antigenic protein that is or is derived from GPC, wherein the GPC is a fragment of a GPC protein.

According to preferred embodiments, the RNA of the first aspect comprises
at least one coding sequence encoding at least one antigenic peptide or protein derived from LASV GPC comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 1-254 or a fragment or variant of any of these sequences;
at least one coding sequence encoding at least one antigenic peptide or protein derived from LASV NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 7547-7797 or a fragment or variant of any of these sequences; or
at least one coding sequence encoding at least one antigenic peptide or protein derived from LASV Z comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 11166-11347 or a fragment or variant of any of these sequences.

Additional information regarding each of these suitable amino acid sequences encoding LASV proteins may also be derived from the sequence listing, e.g. the respective NCBI Accession number, strain and clade information, are provided therein under identifier <223>.

According to other embodiments, the RNA of the first aspect encodes at least one antigenic peptide or protein as defined above and additionally at least one further heterologous peptide or protein element.

Suitably, the at least one further peptide or protein element may promote/improve secretion of the antigenic peptide or protein (e.g. via secretory signal peptides), promote/improve anchoring of the antigenic peptide or protein in the plasma membrane (e.g. via transmembrane elements), promote/improve formation of antigen complexes (e.g. via multimerization domains), promote/improve virus-like particle formation (VLP forming sequence). In addition, the RNA of the first aspect may encode peptide linker elements, self-cleaving peptides, immunologic adjuvant sequences or dendritic cell targeting sequences. Suitable multimerization domains may be selected from the list of amino acid sequences according to SEQ ID NOs: 1116-1167 of WO2017/081082, or fragments or variants of these sequences. Suitable transmembrane elements may be selected from the list of amino acid sequences according to SEQ ID NOs: 1228-1343 of WO2017/081082, or fragments or variants of these sequences. Suitable VLP forming sequences may be selected from the list of amino acid sequences according to SEQ ID NOs: 1168-1227 of WO2017/081082, or fragments or variants of these sequences. Suitable peptide linkers may be selected from the list of amino acid sequences according to SEQ ID NOs: 1509-1565 of WO2017/081082, or fragments or variants of these sequences. Suitable self-cleaving peptides may be selected from the list of amino acid sequences according to SEQ ID NOs: 1434-1508 of WO2017/081082, or fragments or variants of these sequences. Suitable immunologic adjuvant sequences may be selected from the list of amino acid sequences according to SEQ ID NOs: 1360-1421 of WO2017/081082, or fragments or variants of these sequences. Suitable dendritic cell targeting sequences may be selected from the list of amino acid sequences according to SEQ ID NOs: 1344-1359 of WO2017/081082, or fragments or variants of these sequences. Suitable secretory signal peptides may be selected from the list of amino acid sequences according to SEQ ID NOs: 1-1115 and SEQ ID NO: 1728 of WO2017/081082, or fragments or variants of these sequences. Further suitable secretory signal peptides are selected from: SEQ ID NOs: 423-427 of patent application WO2017/070624A1 or a fragment or variant of any of these sequences. In this context SEQ ID NOs: 423-427, of patent application WO2017/070624A1, and the disclosure related thereto, are herewith incorporated by reference.

In embodiments, the RNA of the first aspect encodes at least one antigenic peptide or protein or a fragment thereof as defined above and additionally at least one further heterologous secretory signal peptide.

Such signal peptides are sequences, which typically exhibit a length of about 15 to 30 amino acids and are preferably located at the N-terminus of the encoded peptide, without being limited thereto. Signal peptides as defined herein preferably allow the transport of the antigenic peptide or protein as encoded by the inventive mRNA into a defined cellular compartment, preferably the cell surface, the endoplasmic reticulum (ER) or the endosomal-lysosomal compartment. Examples of secretory signal peptide sequences as defined herein include, without being limited thereto, signal sequences of classical or non-classical MHC-molecules (e.g. signal sequences of MHC I and II molecules, e.g. of the MHC class I molecule HLA-A*0201), signal sequences of cytokines or immunoglobulines, signal sequences of the invariant chain of immunoglobulines or antibodies, signal sequences of Lamp1, Tapasin, Erp57, Calretikulin, Calnexin, PLAT, EPO or albumin and further membrane associated proteins or of proteins associated with the endoplasmic reticulum (ER) or the endosomal-lysosomal compartment.

Particularly preferred signal peptides are those derived from human HLA-A2 (amino acids 1-24), human PLAT (amino acids 1-23, 1-21 or 1-22), human EPO (amino acids 1-27), human ALB (amino acids 1-18), human IgE, human CD5 (amino acids 1-24), human IL2 (amino acids 1-20), human CTRB2 (amino acids 1-18), human IgG-HC (amino acids 1-19), human Ig-HC (amino acids 1-19), human Ig-LC (amino acids 1-19), Gaussia princeps Luc (amino acids 1-17), mouse lgkappa, NrChit1 (1-26), CILp1.1(1-21), Nepenthes rafflesiana Nep1 (amino acids 1-24), human Azul (amino acids 1-19), human CD33 (amino acids 1-16), Vibrio cholera CtxB (amino acids 1-19), human CST4 (amino acids 1-20), human Ins-isol (amino acids 1-24), human SPARC (amino acids 1-17), or Influenza A SP-H1 N1 (Netherlands2009)-HA.

Such signal peptides are preferably used in order to promote secretion of the encoded antigenic peptide or protein. More preferably, a signal peptide as defined herein is fused to an encoded antigenic peptide or protein as defined herein.

Accordingly, in particularly preferred embodiments, the RNA of the first aspect encodes at least one antigenic peptide or protein or a fragment thereof as defined above and additionally at least one further heterologous secretory signal peptide. Said signal peptide is preferably fused to the at least one antigenic peptide or protein, more preferably to the N-terminus of the at least one antigenic peptide or protein as described herein, wherein the signal peptide preferably comprises or consists of an amino acid sequence as defined by SEQ ID NOs: 13880-13913, or a variant or fragment of any one of these amino acid sequences. Such variants or fragments are preferably functional, i.e. exhibit the same desired biological function as the signal peptides they are derived from, and are thus preferably capable of mediating secretion of the fused antigenic protein or peptide.

Suitably, the secretory signal peptide is or is derived from tissue plasminogen activator (TPA or HsPLAT), human serum albumin (HSA or HsALB), or immunoglobulin IgE (IgE).

In preferred embodiments, the secretory signal peptide is or is derived from HsPLAT, HsALB, or IgE, wherein the amino acid sequence of said heterologous signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 13881, 13883, or 13884, or fragment or variant of any of these.

In preferred embodiments, the RNA of the first aspect encodes at least one antigenic peptide or protein or a fragment thereof derived from NP comprising preferably of at least one amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 7547-7797 or a fragment or variant of any of these sequences and additionally at least one further heterologous secretory signal peptide.

In embodiments where the coding RNA of the invention additionally encodes heterologous secretory signal peptides, it is particularly preferred and suitable to generate a fusion protein comprising a heterologous N-terminal secretory signal peptide and a C-terminal peptide or protein derived from NP. The first natural methionine of NP (preferably according to SEQ ID NOs: SEQ ID NO: 7547-7797) is preferably replaced by the heterologous signal sequence. Accordingly, improved secretion of the LASV protein, preferably the NP protein, upon administration of the coding RNA of the first aspect, may be advantageous for the induction of humoral immune responses against the encoded LASV antigenic protein.

Suitable examples of LASV constructs comprising an N-terminal heterologous secretory signal peptide are SP- HsPLAT_NP, SP-HsALB_NP, SP-IgE_NP. The corresponding amino acid sequences for each of the above listed constructs can be found in Table 4.1, 4.2 and 4.3 respectively.

According to preferred embodiments, the RNA of the first aspect comprises
at least one coding sequence encoding at least one antigenic peptide or protein derived from HsPLAT_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 17968-18001 or a fragment or variant of any of these sequences; or
at least one coding sequence encoding at least one antigenic peptide or protein derived from HsALB_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 19328-19361 or a fragment or variant of any of these sequences;
at least one coding sequence encoding at least one antigenic peptide or protein derived from IgE_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 20689-20722 or a fragment or variant of any of these sequences.

Additional information regarding each of these suitable amino acid sequences encoding LASV proteins may also be derived from the sequence listing, e.g. the respective NCBI Accession Number, strain and clade information, are provided therein under identifier <223>.

On the surface of the virion GPC forms a trimer of heterotrimers, each containing the receptor binding subunit GP1 and the transmembrane fusion-mediating subunit GP2, and a stable signal peptide (SSP). GPC precursor is trafficked from the endoplasmic reticulum to the Golgi, where it is N-glycosylated and processed by cellular proteases into its mature form, which comprises non-covalently linked GP1, GP2, and SSP. A study of more than 100 antibodies from human survivors of LASV infection found that the majority of neutralizing response to LASV targeted the prefusion GPC trimer, rather than either subunit alone (Robinson, James E., et al. “Most neutralizing human monoclonal antibodies target novel epitopes requiring both Lassa virus glycoprotein subunits.” Nature communications 7 (2016): 11544). This indicates that to elicit potent neutralizing antibody response against the metastable LASV GP the stabilization in the prefusion conformation is important. Hence, vaccines designed with the intent to elicit neutralizing antibodies, should focus on stabilizing the LASV GPC in its prefusion, metastable state. Perfusion GPC trimer may therefore represent a particularly suitable antigen in the context of the invention. Hastie et al presented recently a crystal structure of the prefusion GPC trimer of LASV, in complex with the human neutralizing antibody 37.7H, which is directed against the quaternary GPC-B epitope (Hastie, Kathryn M., et al. “Structural basis for antibody-mediated neutralization of Lassa virus.” Science 356.6341 (2017): 923-928).

According to preferred embodiments, the at least one antigenic peptide or protein is derived from GPC, preferably from full-length GPC, as GPC is the sole antigen on the viral surface and the primary target of protective humoral immune responses. Moreover, the full-length GPC is required to generate a GPC trimer after translation of the RNA of the first aspect in a cell, requiring GP1, GP2 and stable signal peptide (SSP).

According to preferred embodiments, the at least one antigenic peptide or protein is derived from a prefusion- stabilized GPC.

Preferred prefusion-stabilized GPC comprise at least one of the following mutations i), ii), and iii):
i) amino acid substitutions allowing a covalent link of GP1 and GP2, preferably by introduction of two additional cysteine residues, preferably at position 207 or 206 and 360 or 359, preferably by amino acid substitutions R2070 and G360C;
ii) amino acid substitutions allowing stabilization of GP2, preferably E329P or E328P;
iii) replacing the native GP1-GP2 cleavage site with a furin cleavage site, e.g. RRLL to RRRR, preferably by amino acid substitutions L258R, L259R or L257R, L258R, wherein prefusion-stabilized GPC preferably comprises i), ii), and iii).

Prefusion-stabilized GPC comprises at least one of the following mutations A, preferably of Al, A2, A3 and B, and C:
A amino acid substitutions allowing a covalent link of different structural elements of GPC, preferably by introduction of two additional cysteine residues, wherein preferably
A1 the amino acid substitutions allowing a covalent link of GP1 and GP2, preferably by introduction of two additional cysteine residues, preferably at position 207 or 206 and 360 or 359, preferably by amino acid substitutions R2070 or R2060 and G360C or G359C, or
A2 the amino acid substitutions allowing a covalent link of GP1 to HR1 of GP2, preferably by introduction of two additional cysteine residues, preferably at position 81 or 80 and 319 or 318, preferably by amino acid substitutions T81C or T800 and N319C or N318C, or
A3 the amino acid substitutions allowing a covalent link of T-loop in GP2 to adjacent loop in GP2, preferably by introduction of two additional cysteine residues, preferably at positon 370 or 369 and 386 or 385, preferably by amino acid substitutions W370C or W369C and W386C or W385C,
B amino acid substitutions allowing stabilization of GP2, preferably by introduction of a helix-breaking point mutation in the metastable region of HR1 of GP2 subunit, more preferably by introducing E329P or E328P,
C replacing the native GP1-GP2 cleavage site (S1 P) with a furin cleavage site, e.g. RRLL to RRRR to facilitate efficient processing of the mRNA encoded antigenic protein in target cells, preferably by amino acid substitutions L258R, L259R or L257R, L258R,
wherein prefusion-stabilized GPC preferably comprises at least one mutation A selected from A1, A2 and A3.

According to preferred embodiments, the at least one antigenic peptide or protein is derived from a prefusion-stabilized GPC, wherein the stabilized GPC comprises the following mutations: A1, B, and C (herein referred to as “GPCmutl” or “GPCstabilized”), A2, B and C (herein referred to as “GPCmut2”), A3, B and C (herein referred to as “GPCmut3”), A2, A3, B and C (herein referred to as “GPCmut4”), A1 and B (herein referred to as “GPCmut5”), A2 and B (herein referred to as “GPCmut6”), A3 and B (herein referred to as “GPCmut7”), A2, A3 and B (herein referred to as “GPCmut8”), Al (herein referred to as “GPCmut9”), A2 (herein referred to as “GPCmutl 0”), A3 (herein referred to as “GPCmut11”), or A2 and A3 (herein referred to as “GPCmut12”). Table X3 summarizes the prefusion-stabilized GPC mutants.

According to preferred embodiments, the at least one antigenic peptide or protein is derived from a prefusion-stabilized GPC, wherein the stabilized GPC comprises the following mutations: A1 and/ or A2, wherein A1, B, and C (herein referred to as “GPCmut1”), A2, B and C (herein referred to as “GPCmut2”), A1 and B (herein referred to as “GPCmut5”), A2 and B (herein referred to as “GPCmut6”), A1 (herein referred to as “GPCmut9”), A2 (herein referred to as “GPCmut10”) are particularly preferred.

According to further preferred embodiments, the at least one antigenic peptide or protein is derived from a prefusion-stabilized GPC with at least mutation A1: wherein A1, B, and C (herein referred to as “GPCmut1, A1 and B (herein referred to as “GPCmut5”), A1 (herein referred to as “GPCmut9”), are particularly preferred.

According to further preferred embodiments, the at least one antigenic peptide or protein is derived from a prefusion-stabilized GPC with at least mutation A2, wherein, A2, B and C (herein referred to as “GPCmut2”), A2 and B (herein referred to as “GPCmut6”), and A2 (herein referred to as “GPCmut10”) are particularly preferred.

TABLE X3
Preferred prefusion-stabilized GPC mutants

| Construct | Comprised | | Protein |
|---|---|---|---|
| name | mutations | amino acid position of mutations | SEQ ID NOs |
| GPCmut1 | A1, B, C | R207C, L258R, L259R, E329P, G360C or | 3567-3820 |
| | | R206C, L257R, L258R, E328P, G359C | |
| GPCmut2 | A2, B, C | T81C, L258R, L259R, N319C, E329P or | 22949-22980 |
| | | T80C, L257R, L258R, N318C, E328P | |
| GPCmut3 | A3, B, C | L258R, L259R, E329P, W370C, W386C or | 24645-24676 |
| | | L257R, L258R, E328P, W369C, W385C | |
| GPCmut4 | A2, A3, B, C | T81C, L258R, L259R, N319C, E329P, | 26341-26372 |
| | | W370C, W386C or T80C, L257R, L258R, | |
| | | N318C, E328P, W369C, W385C | |
| GPCmut5 | A1, B | R207C, E329P, G360C or | 28037-28068 |
| | | R206C, E328P, G359C | |
| GPCmut6 | A2, B | T81C, N319C, E329P or | 29733-29764 |
| | | T80C, N318C, E328P | |
| GPCmut7 | A3, B | E329P, W370C, W386C or | 31429-31460 |
| | | E328P, W369C, W385C | |
| GPCmut8 | A2, A3, B | T81C, N319C, E329P, W370C, W386C or | 33125-33156 |
| | | T80C, N318C, E328P, W369C, W385C | |
| GPCmut9 | A1 | R207C, G360C or | 34821-34852 |
| | | R206C, G359C | |
| GPCmut10 | A2 | T81C, N319C or | 36517-36548 |
| | | T80C, N318C | |
| GPCmut11 | A3 | W370C, W386C or | 38213-38244 |
| | | W369C, W385C | |
| GPCmut12 | A2, A3 | T81C, N319C, W370C, W386C or | 39909-39940 |
| | | T80C, N318C, W369C, W385C | |

It has to be noted that under identifier <223> of the sequence listing, respective prefusion-stabilized GPC constructs are referred to as “GPCmut1”, “GPCmut2”, “GPCmut3”, “GPCmut4”, “GPCmut5”, “GPCmut6”, “GPCmut7”, “GPCmut8”, “GPCmut9”, “GPCmut10”, “GPCmut11”, “GPCmut12”, and “GPCstabilized”.

Preferred prefusion-stabilized GPC comprise at least one of the following mutations i), ii), and iii):
i) amino acid substitutions allowing a covalent link of GP1 and GP2, preferably by introduction of two additional cysteine residues, preferably at position 207 or 206 and 360 or 359, preferably by amino acid substitutions R2070 and G3600;
ii) amino acid substitutions allowing stabilization of GP2, preferably E329P or E328P;
iii) replacing the native GP1-GP2 cleavage site with a furin cleavage site, e.g. RRLL to RRRR, preferably by amino acid substitutions L258R, L259R or L257R, L258R, wherein prefusion-stabilized GPC preferably comprises i), ii), and iii).

The introduction of two additional cysteine residues in i) is intended to covalent link of GP1 and GP2 which may lead to a stabilization of the prefusion GPC trimer. Amino acid substitution according to ii) may lead to a stabilization of GP2. Amino acid substitution according to iii) may improve the processing/maturation of GPC.

According to preferred embodiments, the RNA of the first aspect comprises
at least one coding sequence encoding at least one antigenic peptide or protein derived from LASV prefusion-stabilized GPC (“GPCmut1” or “GPCstabilized”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 3567-3820 or a fragment or variant of any of these sequences;
at least one coding sequence encoding at least one antigenic peptide or protein derived from LASV prefusion-stabilized GPC (“GPCmut2”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 22949-22980 or a fragment or variant of any of these sequences;
at least one coding sequence encoding at least one antigenic peptide or protein derived from LASV prefusion-stabilized GPC (“GPCmut3”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 24645-24676 or a fragment or variant of any of these sequences;
at least one coding sequence encoding at least one antigenic peptide or protein derived from LASV prefusion-stabilized GPC (“GPCmut4”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 26341-26372 or a fragment or variant of any of these sequences;
at least one coding sequence encoding at least one antigenic peptide or protein derived from LASV prefusion-stabilized GPC (“GPCmut5”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 28037-28068 or a fragment or variant of any of these sequences;
at least one coding sequence encoding at least one antigenic peptide or protein derived from LASV prefusion-stabilized GPC (“GPCmut6”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 29733-29764 or a fragment or variant of any of these sequences;
at least one coding sequence encoding at least one antigenic peptide or protein derived from LASV prefusion-stabilized GPC (“GPCmut7”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 31429-31460 or a fragment or variant of any of these sequences;
at least one coding sequence encoding at least one antigenic peptide or protein derived from LASV prefusion-stabilized GPC (“GPCmut8”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 33125-33156 or a fragment or variant of any of these sequences;
at least one coding sequence encoding at least one antigenic peptide or protein derived from LASV prefusion-stabilized GPC (“GPCmut9”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 34821-34852 or a fragment or variant of any of these sequences;
at least one coding sequence encoding at least one antigenic peptide or protein derived from LASV prefusion-stabilized GPC (“GPCmut10”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 36517-36548 or a fragment or variant of any of these sequences;
at least one coding sequence encoding at least one antigenic peptide or protein derived from LASV prefusion-stabilized GPC (“GPCmut11”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 38213-38244 or a fragment or variant of any of these sequences, or
at least one coding sequence encoding at least one antigenic peptide or protein derived from LASV prefusion-stabilized GPC (“GPCmut12”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 39909-39940 or a fragment or variant of any of these sequences.

Additional information regarding each of these suitable amino acid sequences encoding LASV proteins may also be derived from the sequence listing, e.g. the respective NCBI Accession No., are provided therein under identifier <223>.

In preferred embodiments, the at least one antigenic peptide or protein is derived from glycoprotein precursor (GPC) or a prefusion-stabilized GPC, or a variant, fragment, or combination thereof. In embodiments, GPC or prefusion-stabilized GPC are truncated proteins.

A “truncated GPC” ora “truncated prefusion-stablized GPC” has to be understood as an N-terminal and/or a C-terminal truncated version of a full-length GPC or prefusion-stabilized GPC protein that typically comprises 490 amino acids (amino acid 1 to amino acid 490) or 491 amino acids (amino acid 1 to amino acid 491). In the context of the invention, the N- and/or C-terminal truncation has to be selected by the skilled person in a way that no important T-cell and/or B-cell epitopes are removed. Suitably, a “truncated protein” of a GPC or of a prefusion-stabilized GPC″ is large enough to elicit an adaptive immune response in a subject (wherein, in the context of the invention, the truncated protein is provided by the RNA comprising the at least one coding sequence encoding at least one antigenic truncated protein).

In preferred embodiments, the truncated GPC or prefusion-stabilized GPC is C-terminally truncated, preferably lacking the cytoplasmic tail.

The deletion of the cytoplasmic tail may lead to an increased surface expression which thereby promotes an increased antigen presentation to B cells with improved immune responses. According to domain prediction analysis tools, the cytoplasmic domain/cytoplasmic tail of GPC is located between amino acid 453 to amino acid 491 or between amino acid 452 to amino acid 490 (the same applies to prefusion-stabilized GPCs) (see e.g. FIG. 1A ofWatanabe, Yasunori, et al. “Structure of the Lassa virus glycan shield provides a model for immunological resistance.” Proceedings of the National Academy of Sciences 115.28 (2018): 7320-7325). Accordingly, the C-terminally truncated GPC or prefusion-stabilized GPC preferably lacks the cytoplasmic tail comprising preferably amino acids 453 to 491 or 452 to 490.

In further preferred embodiments, the truncated protein is a truncated GPC, preferably lacking the cytoplasmic tail, herein referred to as “GPCmut13”.

According to preferred embodiments, the RNA of the first aspect comprises at least one coding sequence encoding at least one antigenic peptide or protein derived from LASV GPC lacking the cytoplasmic tail (“GPCmut13”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 41605-41636 or a fragment or variant of any of these sequences.

According to embodiments, the at least one antigenic peptide or protein is derived from a prefusion-stabilized GPC, wherein the stabilized GPC comprises at least one of the mutations A1, A2, A3, B, and C and is a truncated prefusion-stabilized GPC lacking the cytoplasmic tail.

According to embodiments, the at least one antigenic peptide or protein is derived from a truncated prefusion-stabilized GPC lacking the cytoplasmic tail, wherein the truncated stabilized GPC comprises the following mutations: Al, B, and C (herein referred to as “GPCmut14”), A2, B and C (herein referred to as “GPCmut15”), A3, B and C (herein referred to as “GPCmut16”), A2, A3, B and C (herein referred to as “GPCmut17”), A2 and B (herein referred to as “GPCmut18”), A2 and B (herein referred to as “GPCmut19”), A3 and B (herein referred to as “GPCmut20”), A2, A3 and B (herein referred to as “GPCmut21”), A1 (herein referred to as “GPCmut22”),

A2 (herein referred to as “GPCmut23”), A3 (herein referred to as “GPCmut24”), or A2 and A3 (herein referred to as “GPCmut25”).

In various embodiments, the amino acid sequences of the at least one antigenic peptide or protein derived from LASV, in particular from LASV GPC or prefusion-GPC, is mutated/substituted to delete at least one predicted or potential glycosylation site.

It may suitable in the context of the invention that glycosylation sites in the encoded amino acid sequence are mutated/substituted which means that encoded amino acids which may be glycosylated, e.g. after translation of the coding RNA upon in vivo administration, are exchanged to a different amino acid. Accordingly, on nucleic acid level, codons encoding e.g. asparagine which are predicted to be glycosylated (N glycosylation sites) are substituted with codons encoding glutamine.

In embodiments, the coding region encoding at least one GPC or prefusion-stabilized GPC, or a fragment, variant or derivative thereof, is mutated in a way to delete at least one predicted or potential glycosylation site. Glycosylation is an important post-translational or co-translational modification of proteins. The majority of proteins synthesized in the rough endoplasmatic reticulum (ER) undergoes glycosylation. There are mainly two types of glycosylation: a) In N-glycosylation, the addition of sugar chains takes place at the amide nitrogen on the side-chain of the asparagine or arginine. b) In O-glycosylation, the addition of sugar chains takes place on the hydroxyl oxygen on the side-chain of hydroxylysine, hydroxyproline, serine, tyrosine or threonine.

Moreover, phospho-glycans linked through the phosphate of a phospho-serine and C-linked glycans, a rare form of glycosylation where a sugar is added to a carbon on a tryptophan side-chain, are known. The often inadequate antibody immune response elicited by natural LASV infection has been, in part, attributed to the abundance of N-linked glycosylation on the GPC presented on the virions with the glycans most likely impairing antibody access to neutralizing epitopes.

Therefore, it is particularly advantageous to delete the potential glycosylation sites of the encoded LASV protein, in particular GPC or prefusion-stabilized GPC. By mutation/substitution of the relevant amino acids, the glycosylation may be prevented. In this context at least one codon coding for an asparagine, arginine, serine, threonine, tyrosine, lysine, proline or tryptophan is modified in such a way that a different amino acid is encoded thereby deleting at least one predicted or potential glycosylation site. The predicted glycosylation sites may be predicted by using artificial neural networks that examine the sequence for common glycosylation sites, e.g. N-glycosylation sites may be predicted by using the NetNGlyc 1.0 Server.

In preferred embodiments, the at least one antigenic protein from LASV, preferably of GPC or prefusion- stabilized GPC, is mutated to delete at least one predicted or potential glycosylation site, e.g. asparagine (N) is substituted by a glutamine (Q). Accordingly, on nucleic acid level, the nucleic acid sequence is modified to encode for Q instead of N at predicted N-glycosylation sites, for example at predicted N-glycosylation sites of the encoded GPC or prefusion-stabilized GPC protein, or a fragment, variant or derivative thereof. In this context the term “mutated GPC” or “mutated prefusion-GPC” means that at least one (predicted) glycosylation site is mutated.

In particularly preferred embodiments, the at least one GPC or prefusion-stabilized GPC is mutated to substitute at least one asparagine (N) by a glutamine (Q) in GP1 of GPC or prefusion-stabilized GPC, preferably at amino acid positions 99 (or 98) or 119 (or 118).

In various embodiments, the amino acid sequences of the at least one antigenic peptide or protein from LASV, in particular LASV GPC or prefusion-stabilized GPC, is mutated to delete all predicted or potential glycosylation sites.

Accordingly, it may be particularly preferred that all predicted glycosylation sites of the amino acid sequences of the at least one antigenic peptide or protein, in particular LASV GPC or prefusion-stabilized GPC are mutated to completely prevent glycosylation of the resulting protein or peptide. This aspect of the invention may apply for e.g. all N-glycosylation sites or for all 0-glycosylation sites or for all glycosylation sites irrespective of their biochemical nature.

According to preferred embodiments, the RNA comprises at least one coding sequence encoding at least one antigenic peptide or protein derived from a LASV protein as specified herein, or fragments and variants thereof. In that context, any coding sequence encoding at least one antigenic peptide or protein derived from LASV protein or fragments and variants thereof may be understood as suitable coding sequence and may therefore be comprised in the RNA of the first aspect.

In preferred embodiments, the RNA of the first aspect may comprise or consist of at least one coding sequence encoding at least one antigenic peptide or protein derived from a LASV protein as defined herein, preferably GPC, prefusion-stabilized GPC, NP, or Z, preferably encoding any one of SEQ ID NO: 1-254, 3567-3820, 7547-7797, 11166-11347, 17968-18001, 19328-19361, 20689-20722, 22949-22980, 24645-24676, 26341-26372, 28037-28068, 29733-29764, 31429-31460, 33125-33156, 34821-34852, 36517-36548, 38213-38244, 41605-41636 or fragments of variants thereof. It has to be understood that, on nucleic acid level, any nucleic acid sequence, in particular, any RNA sequence which encodes an amino acid sequences being identical to SEQ ID NO: 1-254, 3567-3820, 7547-7797, 11166-11347, 17968-18001, 19328-19361, 20689-20722, 22949-22980, 24645-24676, 26341-26372, 28037-28068, 29733-29764, 31429-31460, 33125-33156, 34821-34852, 36517-36548, 38213-38244, 41605-41636 or fragments or variants thereof, or any nucleic acid sequence (e.g. DNA sequence, RNA sequence) which encodes amino acid sequences being at least 70%, 80%, 85%, 86%, 87%, 88%, 89%, 90%, 91%, 92%, 93%, 94%, 95%, 96%, 97%, 98%, or 99% identical to any one of SEQ ID NO: 1-254, 3567-3820, 7547-7797, 11166-11347, 17968-18001, 19328-19361, 20689-20722, 22949-22980, 24645-24676, 26341-26372, 28037-28068, 29733-29764, 31429-31460, 33125-33156, 34821-34852, 36517-36548, 38213-38244, 41605-41636 or fragments or variants thereof, may be selected and may accordingly be understood as suitable coding sequence and may therefore be comprised in the RNA of the first aspect.

In particularly preferred embodiments, the RNA of the first aspect comprises at least one coding sequence, wherein, suitably,
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 255-2286 (encoding GPC) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 3821-6106 (encoding GPCmut1) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 22981-23108 (encoding GPCmut2) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 24677-24804 (encoding GPCmut3) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 26373-26500 (encoding GPCmut4) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 28069-28196 (encoding GPCmut5) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 29765-29892 (encoding GPCmut6) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 31461-31588 (encoding GPCmut7) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 33157-33284 (encoding GPCmut8) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 34853-34980 (encoding GPCmut9) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 36549-36676 (encoding GPCmut10) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 38245-38372 (encoding GPCmut11) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 39941-40068 (encoding GPCmut12) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 41637-41764 (encoding GPCmut13) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 7798-9805 (encoding NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 18002-18103 (encoding SP-HsPLAT_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 19362-19463 (encoding SP-HsALB_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 20723-20824 (encoding SP-IgE_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 11348-12803 (encoding Z) or a fragment or a fragment or variant of any of these sequences.

Additional information regarding each of said suitable nucleic acid sequences may also be derived from the sequence listing, in particular from the details provided therein under identifier <223>.

According to preferred embodiments, the RNA is a modified and/or stabilized RNA.

According to preferred embodiments, the RNA of the present invention may thus be provided as a “stabilized RNA” that is to say an RNA showing improved resistance to in vivo degradation and/or an RNA showing improved stability in vivo, and/or an RNA showing improved translatability in vivo. In the following, specific suitable modifications suitably to “stabilize” the RNA are described. Such stabilization may also be effected by providing a “dried RNA” and/or a “purified RNA” as specified herein.

In the following, suitable modifications are described that are capable of “stabilizing” the RNA of the invention.

In embodiments, the RNA is a modified RNA, wherein the modification refers to chemical modifications comprising backbone modifications and/or sugar modifications and/or base modifications.

A backbone modification may be a modification in which phosphates of the backbone of the nucleotides contained in a nucleic acid, e.g. the RNA, are chemically modified. A sugar modification may be a chemical modification of the sugar of the nucleotides of the RNA as defined herein. Furthermore, a base modification may be a chemical modification of the base moiety of the nucleotides of the RNA. In this context, nucleotide analogues or modifications are preferably selected from nucleotide analogues which are applicable for RNA in vitro transcription and/or in vivo translation.

In particularly preferred embodiments of the present invention, the nucleotide analogues/modifications which may be incorporated into a modified nucleic acid or particularly into a modified RNA as described herein are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

Particularly preferred are pseudouridine (ψ), N1-methylpseudouridine (m1ψ), 5-methylcytosine, and 5-methoxyuridine. Accordingly, the RNA of the first aspect may comprise at least one modified nucleotide selected from pseudouridine (ψ), N1-methylpseudouridine (m1ψ), 5-methylcytosine, and 5-methoxyuridine, wherein pseudouridine (ψ), and N1-methylpseudouridine (m1ψ) are particularly preferred.

In preferred embodiments, the RNA of the invention comprises at least one coding sequence, wherein the at least one coding sequence is a codon modified coding sequence.

In preferred embodiments, the at least one coding sequence of the RNA of the first aspect is a codon modified coding sequence, wherein the amino acid sequence encoded by the at least one codon modified coding sequence is preferably not being modified compared to the amino acid sequence encoded by the corresponding wild type coding sequence.

The term “codon modified coding sequence” relates to coding sequences that differ in at least one codon (triplets of nucleotides coding for one amino acid) compared to the corresponding wild type coding sequence. Suitably, a codon modified coding sequence in the context of the invention may show improved resistance to in vivo degradation and/or improved stability in vivo and/or improved translatability in vivo. Codon modifications in the broadest sense make use of the degeneracy of the genetic code wherein multiple codons may encode the same amino acid and may be used interchangeably (cf. Table 1) to optimize/modify the coding sequence of the RNA for in vivo applications.

In particularly preferred embodiments of the first aspect, the at least one sequence is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof.

According to preferred embodiments, the RNA may be modified, wherein the C content of the at least one coding sequence may be increased, preferably maximized, compared to the C content of the corresponding wild type coding sequence (herein referred to as “C maximized coding sequence”). The amino acid sequence encoded by the C maximized coding sequence of the RNA is preferably not modified as compared to the amino acid sequence encoded by the respective wild type nucleic acid coding sequence. The generation of a C maximized nucleic acid sequences may suitably be carried out using a method according to WO2015/062738. In this context, the disclosure of WO2015/062738 is included herewith by reference. Throughout the disclosure of the invention, including the <223> identifier of the sequence listing, C maximized coding sequences are indicated by the abbreviation “opt2”.

According to preferred embodiments, the RNA may be modified, wherein the G/C content of the at least one coding sequence may be modified or optimized compared to the G/C content of the corresponding wild type coding sequence (herein referred to as “G/C content modified coding sequence”). The terms “G/C optimization” or “G/C content modification” relate to a nucleic acid that comprises a modified, preferably an increased number of guanosine and/or cytosine nucleotides as compared to the corresponding wild type nucleic acid sequence. In particular, in case of RNA, sequences having an increased G (guanosine)/C (cytosine) content are more stable than sequences having an increased A (adenosine)/U (uracil) content. “Optimized” in that context refers to a coding sequence wherein the G/C content is preferably increased to the essentially highest possible G/C content. The amino acid sequence encoded by the G/C content modified/optimized coding sequence of the nucleic acid sequence is preferably not modified as compared to the amino acid sequence encoded by the respective wild type nucleic acid coding sequence. In that context, the G/C content of the coding sequence of the RNA sequence of the present invention is increased by at least 10%, 20%, 30%, or 40% compared to the G/C content of the coding sequence of the corresponding wild type nucleic acid sequence (e.g. RNA sequence). The generation of a G/C content optimized nucleic RNA sequence may suitably be carried out using a G/C content optimization method according to WO2002/098443. In this context, the disclosure of WO2002/098443 is included in its full scope in the present invention. Throughout the disclosure of the invention, including the <223> identifier of the sequence listing, G/C optimized and G/C content modified coding sequences are indicated by the abbreviation “opt1, opt5, opt6, opt11”.

According to embodiments, the RNA may be modified, wherein codons in the at least one coding sequence may be adapted to human codon usage (herein referred to as “human codon usage adapted coding sequence”). Codons encoding the same amino acid occur at different frequencies in humans. Accordingly, the coding sequence of the RNA is preferably modified such that the frequency of the codons encoding the same amino acid corresponds to the naturally occurring frequency of that codon according to the human codon usage. E.g., in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table 1). Accordingly, such a procedure (as exemplified for Ala) is applied for each amino acid encoded by the coding sequence of the RNA to obtain sequences adapted to human codon usage. Throughout the disclosure of the invention, including the <223> identifier of the sequence listing, human codon usage adapted coding sequences are indicated by the abbreviation “opt3”.

TABLE 1
Human codon usage table with frequencies
indicated for each amino acid

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

*most frequent human codon

According to embodiments, the RNA of the present invention may be modified, wherein the codon adaptation index (CAI) may be increased or preferably maximised in the at least one coding sequence (herein referred to as “CAI maximized coding sequence”). Accordingly, it is preferred that most codons of the wild type nucleic acid sequence that are relatively rare in e.g. a human cell are exchanged for a respective codon that is frequent in the e.g. a human cell, wherein the frequent codon encodes the same amino acid as the relatively rare codon. Suitably, the most frequent codons are used for each encoded amino acid (see Table 1, most frequent human codons are marked with asterisks (*)). Suitably, the RNA comprises at least one coding sequence, wherein the codon adaptation index (CAI) of the at least one coding sequence is at least 0.5, 0.8, 0.9 or 0.95. Most preferably, the codon adaptation index (CAI) of the at least one coding sequence is 1. E.g., in the case of the amino acid Ala, the wild type coding sequence is adapted in a way that the most frequent human codon “GCC” is always used for said amino acid. Accordingly, such a procedure (as exemplified for Ala) is applied for each amino acid encoded by the coding sequence of the RNA to obtain CAI maximized coding sequences. Throughout the disclosure of the invention, including the <223> identifier of the sequence listing, CAI maximized coding sequences are indicated by the abbreviation “opt4”.

Accordingly, in a particularly preferred embodiment, the RNA of the first aspect comprises at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to any one SEQ ID NOs: 509-2286 (encoding GPC) or a fragment or variant of any of these sequences, or at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 4075-6106 (encoding GPCmut1) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 22981-23108 (encoding GPCmut2) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 24677-24804 (encoding GPCmut3) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 26373-26500 (encoding GPCmut4) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 28069-28196 (encoding GPCmut5) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 29765-29892 (encoding GPCmut6) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 31 461-31 588 (encoding GPCmut7) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 33157-33284 (encoding GPCmut8) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 34853-34980 (encoding GPCmut9) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 36549-36676 (encoding GPCmut10) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 38245-38372 (encoding GPCmut11) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 39941-40068 (encoding GPCmut12) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 41637-41764 (encoding GPCmut13) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 8049-9805 (encoding NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 18002-18103, 19362-19463, or 20723-20824 (encoding SP-NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 11530-12803 (encoding Z) or a fragment or variant of any of these sequences.

Additional information regarding each of these suitable nucleic acid sequences encoding may also be derived from the sequence listing, in particular from the details provided therein under identifier <223>.

In particularly preferred embodiment, the an RNA of the first aspect comprises at least one coding sequence comprising a G/C optimized or G/C content modified coding sequence (opt1, opt5, opt6, opt11) comprising a nucleic acid sequence which is identical or at least 50%, 60%, 70%, 80%, 85%, 86%, 87%, 88%, 89%, 90%, 91%, 92%, 93%, 94%, 95%, 96%, 97%, 98%, or 99% identical to any one of the nucleic acid sequences according to the SEQ ID NOs: 509-762, 1525-2286 (encoding GPC) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 4075-4582, 5345-6106 (encoding GPCmut1) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 22981-23044, 23077-23108 (encoding GPCmut2) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 24677-24740, 24773-24804 (encoding GPCmut3) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 26373-26436, 26469-26500 (encoding GPCmut4) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 28069-28132, 28165-28196 (encoding GPCmut5) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 29765-29828, 29861-29892 (encoding GPCmut6) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 31461-31524, 31557-31588 (encoding GPCmut7) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 33157-33220, 33253-33284 (encoding GPCmut8) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 34853-34916, 34949-34980 (encoding GPCmut9) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 36549-36612, 36645-36676 (encoding GPCmut10) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 38245-38308, 38341-38372 (encoding GPCmut11) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 39941-40004, 40037-40068 (encoding GPCmut12) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 41637-41700, 41733-41764 (encoding GPCmut13) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 8049-8299, 9053-9805 (encoding NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 18002-18035, 18070-18103, 19362-19395, 19430- 19463, 20723-20756, 20791-20824 (encoding SP_NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 11530-11711, 12258-12803 (encoding Z) or a fragment or variant of any of these sequences.

Additional information regarding each of these suitable nucleic acid sequences encoding may also be derived from the sequence listing, in particular from the details provided therein under identifier <223>.

In a particularly preferred embodiment, the RNA of the first aspect comprises a ribosome binding site, also referred to as “Kozak sequence”, identical to or at least 80%, 85%, 90%, 95% identical to any one of the sequences according to SEQ ID NOs: 13844 or 13845 or fragments or variants thereof.

In embodiments, the RNA of the first aspect is monocistronic, bicistronic, or multicistronic.

In preferred embodiments, the RNA of the invention is monocistronic.

The term “monocistronic” will be recognized and understood by the person of ordinary skill in the art, and is e.g. intended to refer to an RNA that comprises only one coding sequences as defined herein. The terms “bicistronic” or “multicistronic” as used herein will be recognized and understood by the person of ordinary skill in the art, and are e.g. intended to refer to an RNA that may comprise two (bicistronic) or even more (multicistronic) coding sequences as defined herein.

In embodiments, the RNA is monocistronic and the coding sequence of said monocistronic RNA encodes at least two different antigenic peptides or proteins derived from a LASV protein as defined herein, or a fragment or variant thereof. Accordingly, the at least one coding sequence of the monocistronic RNA may encode at least two, three, four, five, six, seven, eight and more antigenic peptides or proteins derived from a LASV protein, wherein the at least two, three, four, five, six, seven, eight and more antigenic peptides or proteins may be linked with or without an amino acid linker sequence, wherein said linker sequence can comprise rigid linkers, flexible linkers, cleavable linkers (self-cleaving peptides) as defined above, or a combination thereof (herein referred to as “multi-antigen-constructs/nucleic acid”).

In embodiments, the RNA of the invention is bicistronic or multicistronic and comprises at least two coding sequences, wherein the at least two coding sequences encode two or more different antigenic peptides or proteins derived from a LASV protein, or a fragment or variant of any of these. Accordingly, the coding sequences in a bicistronic or multicistronic RNA may encode distinct antigenic proteins or peptides as defined herein or a fragment or variant thereof. Preferably, the coding sequences in said bicistronic or multicistronic RNA may be separated by at least one IRES (internal ribosomal entry site) sequence. Thus, the term “encoding two or more antigenic peptides or proteins” may mean, without being limited thereto, that the bicistronic or multicistronic RNA encodes e.g. at least two, three, four, five, six or more (preferably different) antigenic peptides or proteins of different LASV or their fragments or variants within the definitions provided herein. Alternatively, the bicistronic or multicistronic RNA may encode e.g. at least two, three, four, five, six or more (preferably different) antigenic peptides or proteins derived from the same LASV or fragments or variants within the definitions provided herein. In that context, suitable IRES sequences may be selected from the list of nucleic acid sequences according to SEQ ID NOs: 1566-1662 of WO2017/081082, or fragments or variants of these sequences. In this context, the disclosure of WO2017/081082 relating to IRES sequences is herewith incorporated by reference.

It has to be understood that in the context of the invention, certain combinations of coding sequences may be generated by any combination of monocistronic, bicistronic and multicistronic nucleic acids and/or multi- antigen-constructs/nucleic acid to obtain a nucleic acid composition encoding multiple antigenic peptides or proteins as defined herein (further explained in the context of the second aspect).

Preferably, the RNA comprising at least one coding sequence as defined herein typically comprises a length of about 50 to about 20000, or 500 to about 20000 nucleotides, or about 500 to about 20000 nucleotides, or about 500 to about 10000 nucleotides, or of about 1000 to about 10000 nucleotides, or preferably of about 1000 to about 5000 nucleotides, or even more preferably of about 1000 to about 2500 nucleotides.

According to preferred embodiments, the RNA of the first aspect is a coding RNA, preferably an mRNA, a self-replicating RNA, a circular RNA, or a replicon RNA.

In embodiments, the RNA is a circular RNA. As used herein, “circular RNA” or “circRNA” has to be understood as a circular polynucleotide that can encode at least one antigenic peptide or protein as defined herein. Accordingly, in preferred embodiments, said circular RNA comprises at least one coding sequence encoding at least one antigenic peptide or protein derived from LASV or a fragment or variant thereof. Further, said circRNA may comprise at least one 3′-UTR and/or 5′-UTR as defined herein. The production of circRNAs can be performed using various methods provided in the art. E.g., U.S. Pat. No. 6,210,931 teaches a method of synthesizing circRNAs by inserting DNA fragments into a plasmid containing sequences having the capability of spontaneous cleavage and self-circularization. U.S. Pat. No. 5,773,244 teaches producing circRNAs by making a DNA construct encoding an RNA cyclase ribozyme, expressing the DNA construct as RNA, and then allowing the RNA to self-splice, which produces a circRNA free from intron in vitro. WO1992/001813 teaches a process of making single strand circular nucleic acids by synthesizing a linear polynucleotide, combining the linear nucleotide with a complementary linking oligonucleotide under hybridization conditions, and ligating the linear polynucleotide. The person skilled in the art may also use methods provided in WO2015/034925 or WO2016/011222 to produce circular RNA. Accordingly, methods for producing circular RNA as provided in U.S. Pat. No. 6,210,931, U.S. Pat. No. 5,773,244, WO1992/001813, WO2015/034925 and WO2016/011222 may suitably be used to generate the circRNA of the invention.

In embodiments, the RNA is a replicon RNA. The term “replicon RNA” will be recognized and understood by the person of ordinary skill in the art, and is e.g. intended to be an optimized self-replicating RNA. Such constructs may include replication elements (replicase) derived from e.g. alphaviruses and the substitution of the structural virus proteins with the nucleic acid of interest. Alternatively, the replicase may be provided on an independent nucleic acid construct comprising a replicase sequence derived from e.g. Semliki forest virus (SFV), Sindbis virus (SIN), Venezuelan equine Encephalitis virus (VEE), Ross-River virus (RRV), or other viruses belonging to the alphavirus family. Downstream of the replicase may be a sub-genomic promoter that controls replication of the replicon RNA of the first aspect.

In particularly preferred embodiments the RNA of the first aspect is an mRNA.

The term “mRNA” (abbreviation of “messenger RNA”) will be recognized and understood by the person of ordinary skill in the art, and is e.g. intended to be a ribonucleic acid molecule, i.e. a polymer consisting of nucleotides. These nucleotides are usually connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA usually provides the coding sequence (cds) that is translated into an amino-acid sequence of a particular peptide or protein. Typically, an mRNA comprises a 5′-cap structure, UTR elements, and a 3′ poly(A) sequence.

The RNA of the invention may be prepared using any method known in the art, including chemical synthesis such as e.g. solid phase RNA synthesis, as well as in vitro methods, such as RNA in vitro transcription reactions.

In a preferred embodiment, the RNA, preferably the mRNA is obtained by RNA in vitro transcription.

Accordingly, the RNA of the invention is an in vitro transcribed RNA, preferably an in vitro-transcribed mRNA.

The terms “RNA in vitro transcription” or “in vitro transcription” relate to a process wherein RNA is synthesized in a cell-free in vitro system. RNA may be obtained by DNA-dependent in vitro transcription of an appropriate DNA template, e.g. a linearized plasmid DNA template or a PCR-amplified DNA template. The promoter for controlling RNA in vitro transcription can be any promoter for any DNA-dependent RNA polymerase. Suitable examples of DNA-dependent RNA polymerases are the T7, T3, SP6, or Syn5 RNA polymerases.

Reagents used in RNA in vitro transcription typically include: a DNA template (linearized plasmid DNA or PCR generated DNA) with a promoter sequence that has a high binding affinity for its respective RNA polymerase such as bacteriophage-encoded RNA polymerases (T7, T3, SP6, or Syn5); a nucleotide mixture comprising ribonucleotide triphosphates (NTPs) for the four bases (adenine, cytosine, guanine and uracil) and optionally, a cap analogue as defined herein (preferably a cap° analogue e.g., m7G(5′)ppp(5′)G (m7G) or a cap1 analogue, e.g. m7G(5′)ppp(5′)(2′OMeA)pG or m7G(5′)ppp(5′)(2′OMeG)pG); optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising e.g. TRIS-Citrate as disclosed in WO2017/109161.

In embodiments, the nucleotide mixture used in RNA in vitro transcription may additionally comprise at least one modified nucleotides as defined herein. In that context, preferred modified nucleotides may be selected from pseudouridine N1-methylpseudouridine (m1ψ), 5-methylcytosine, and 5-methoxyuridine. In embodiments, said at least one modified nucleotide at least partially replaces at least one non-modified nucleotide. Preferably, said at least one modified nucleotide completely replaces all of the corresponding non- modified nucleotides in the RNA sequence.

In preferred embodiments, the nucleotide mixture (i.e. the fraction of each nucleotide in the mixture) used for RNA in vitro transcription reactions may be optimized for the given RNA sequence, preferably as described in WO2015/188933.

In embodiment where more than one different RNA as defined herein has to be produced, e.g. where 2, 3, 4, 5, 6, 7, 8, 9, 10 or even more different RNAs have to be produced (e.g. encoding different LASV antigens, e.g. a combination of LASV GPC and LASV NP; see second aspect), procedures as described in WO2017/109134 may be suitably used. In that context, it may be required to optimize/modify the RNA sequences of at least one of the more than one different RNA sequences for HPLC-based purification and/or analysis using procedures as described in PCT patent application PCT/EP2017/078647.

In the context of pharmaceutical RNA production, it may be required to provide GMP-grade RNA. GMP-grade RNA may be produced using a manufacturing process approved by regulatory authorities. Accordingly, in a particularly preferred embodiment, RNA production is performed under current good manufacturing practice (GMP), implementing various quality control steps on DNA and RNA level, preferably according to WO2016/180430. In preferred embodiments, the RNA of the invention is a GMP-grade RNA, particularly a GMP-grade mRNA. The obtained RNA products are preferably purified using PureMessenger® (CureVac, Tübingen, Germany; RP-HPLC according to WO2008/077592) and/or tangential flow filtration (as described in WO2016/193206) and/or oligo d(T) purification.

In further preferred embodiments, the RNA, particularly the purified RNA, is lyophilized, suitably according to WO2016/165831 or WO2011/069586 to yield a temperature stable dried RNA (powder) as defined herein. The RNA of the invention, particularly the purified RNA may also be dried using spray-drying or spray-freeze drying, suitably according to WO2016/184575 or WO2016184576 to yield a temperature stable RNA (powder). Accordingly, in the context of manufacturing and purifying RNA, the disclosures of WO2017/109161, WO2015/188933, WO2016/180430, WO2008/077592, WO2016/193206, WO2016/165831, WO2011/069586, WO2016/184575, and WO2016/184576 are incorporated herewith by reference.

Accordingly, in preferred embodiments, the RNA is a dried RNA, particularly a dried mRNA.

The term “dried RNA” as used herein has to be understood as RNA that has been lyophilized, or spray-dried, or spray-freeze dried as defined above to obtain a temperature stable RNA (e.g. in from of a powder or granules).

In preferred embodiments, the RNA of the invention is a purified RNA, particularly purified mRNA.

The term “purified RNA” or “purified mRNA” as used herein has to be understood as RNA which has a higher purity after certain purification steps (e.g. HPLC, TFF, oligo d(T) purification, precipitation steps) than the starting material (e.g. crude in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences, elongated sequences etc.), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full-length RNA transcripts is as close as possible to 100%. Accordingly “purified RNA” as used herein has a degree of purity of more than 75%, 80%, 85%, 90%, 91%, 92%, 93%, 94%, 95%, 96%, 97%, 98%, 99% or more. The degree of purity may e.g. be determined by an analytical HPLC, wherein the percentages provided above correspond to the ratio between the area of the peak for the target RNA and the total area of all peaks representing the by-products. Alternatively, the degree of purity may e.g. be determined by an analytical agarose gel electrophoresis or capillary gel electrophoresis or mass spectrometry.

It has to be understood that “dried RNA” as defined herein and “purified RNA” as defined herein or “GMP-grade mRNA” as defined herein may have superior stability characteristics (in vitro, in vivo) and improved efficiency (e.g. better translatability of the mRNA in vivo) and are therefore particularly suitable in the context of the invention. Moreover, “dried RNA” as defined herein and “purified RNA” as defined herein or “GMP-grade mRNA” may be particularly suitable for medical use as defined herein.

The RNA may suitably be modified by the addition of a 5′-cap structure, which preferably stabilizes the nucleic acid as described herein.

Accordingly, in preferred embodiments, the RNA of the first aspect comprises a 5′-cap structure, preferably m7G, cap° (e.g. m7G(5′)ppp(5′)G), cap1 (e.g. m7G(5′)ppp(5′)(2′OMeG) or m7G(5′)ppp(5′)(2′OMeA)), cap2, a modified cap0, or a modified cap1 structure (generated using a cap analogue as defined below), wherein cap1 is particularly preferred.

The term “5′-cap structure” as used herein will be recognized and understood by the person of ordinary skill in the art, and is e.g. intended to refer to a modified nucleotide (cap analogue), particularly a guanine nucleotide, added to the 5′-end of an RNA molecule, e.g. an mRNA molecule. Preferably, the 5′-cap is added using a 5′-5′-triphosphate linkage (also named m7GpppN). Further examples of 5′-cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Further 5′-cap structures which may be suitable in the context of the present invention are cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis or RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018075827 and WO2017/066797 wherein the disclosures referring to cap analogues are incorporated herewith by reference.

In embodiments, a modified cap1 structure is generated using a cap analogue as disclosed in WO2017/053297, WO2017/066793, WO2017/066781, WO2017/066791, WO2017/066789, WO2017/066782, WO2018075827 and WO2017/066797. In particular, any cap structures derivable from the structure disclosed in claim 1-5 of WO2017/053297 may be suitably used to co-transcriptionally generate a modified capl structure. Further, any cap structures derivable from the structure defined in claim 1 or claim 21 of WO2018075827 may be suitably used to co-transcriptionally generate a modified cap1 structure.

In preferred embodiments, the 5′-cap structure may suitably be added co-transcriptionally using cap-analogues as defined herein in an RNA in vitro transcription reaction as defined herein. Preferred cap-analogues in the context of the invention are m7G(5′)ppp(5′)G (m7G) or 3′-O-Me-m7G(5′)ppp(5′)G. Further preferred cap-analogues in the context of the invention are m7G(5′)ppp(5′)(2′OMeA)pG or m7G(5′)ppp(5′)(2′OMeG)pG to co-transcriptionally generate cap1 structures.

In other embodiments, the 5′-cap structure is added via enzymatic capping using capping enzymes (e.g. vaccinia virus capping enzymes and/or cap-dependent 2′-O methyltransferases) to generate cap0 or cap1 or cap2 structures. The 5′-cap structure (cap0 or cap1) may be added using immobilized capping enzymes and/or cap-dependent 2′-O methyltransferases using methods and means disclosed in WO2016/193226.

Accordingly, the RNA of the first aspect may comprise a 5′-cap structure, preferably m7G (m7G(5′)), m7G(5′)ppp(5′)(2′OMeA), or m7G(5′)ppp(5′)(2′OMeG).

In that context, it is preferred that the RNA of the invention comprises a cap1 structure as defined above, which preferably result in an increased protein expression through e.g. high capping efficiencies and increased translation efficiencies. Further suitably, the RNA of the invention comprising a cap1 structure displays a decreased stimulation of the innate immune system as compared to cap0 constructs of the same nucleic acid sequence. The person of ordinary skill knows how to determine translation efficiencies, capping degree, and immune stimulation.

In a particularly preferred embodiment, the RNA of the first aspect of the invention comprises a cap1 structure, wherein said cap1 structure may be formed enzymatically or co-transcriptionally (e.g. using m7G(5′)ppp(5′)(2′OMeA)pG or m7G(5′)ppp(5′)(2′OMeG)pG analogues).

In preferred embodiments, the artificial RNA of the first aspect comprises an m7G(5′)ppp(5′)(2′OMeA)pG cap structure. In such embodiments, the coding RNA comprises a 5′ terminal m7G cap, and an additional methylation of the ribose of the adjacent nucleotide of m7GpppN, in that case, a 2′O methylated adenosine.

In other preferred embodiments, the artificial RNA of the first aspect comprises an m7G(5′)ppp(5′)(2′OMeG)pG cap structure. In such embodiments, the coding RNA comprises a 5′ terminal m7G cap, and an additional methylation of the ribose of the adjacent nucleotide, in that case, a 2′O methylated guanosine.

Accordingly, whenever reference is made to suitable RNA or mRNA sequences in the context of the invention, the first nucleotide of said RNA or mRNA sequence, that is the nucleotide downstream of the m7G(5′)ppp structure, may be a 2′O methylated guanosine or a 2′O methylated adenosine.

Accordingly, in other embodiments, the artificial RNA of the invention may comprise a 5′-cap sequence element according to SEQ ID NOs 13846 or 13847 or a fragment or variant thereof.

In preferred embodiments, the RNA of the invention comprises at least one poly(A) sequence, preferably comprising 30 to 150 adenosine nucleotides.

In preferred embodiments, the poly(A) sequence, suitable located at the 3′ terminus (e.g. downstream of the 3′-UTR as defined herein), comprises 10 to 500 adenosine nucleotides, 10 to 200 adenosine nucleotides, 40 to 200 adenosine nucleotides or 40 to 150 adenosine nucleotides. In a particularly preferred embodiment, the poly(A) sequence comprises about 64 adenosine nucleotides. Suitably, the length of the poly(A) sequence may be at least about or even more than about 10, 50, 64, 75, 100, 200, 300, 400, or 500 adenosine nucleotides. In further particularly preferred embodiments, the poly(A) sequence comprises about 75 adenosine nucleotides.

In preferred embodiments, the coding RNA comprises at least one poly(A) sequence comprising about 30 to about 200 adenosine nucleotides. In preferred embodiments, the poly(A) sequence comprises about 64 adenosine nucleotides (A64). In particularly preferred embodiments, the poly(A) sequence comprises about 100 adenosine nucleotides (A100). In particularly preferred embodiments, the poly(A) sequence comprises about 150 adenosine nucleotides.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of an RNA, of up to about 1000 adenosine nucleotides. Preferably, said poly(A) sequence is essentially homopolymeric, e.g. a poly(A) sequence of e.g. 100 adenosine nucleotides has essentially the length of 100 nucleotides. In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide different from an adenosine nucleotide). In the context of the present invention, a poly(A) sequence may be located within an mRNA or any other nucleic acid molecule, such as in a DNA serving as template for the generation of an RNA, preferably an mRNA, e.g., by transcription said DNA template (e.g., plasmid DNA or PCR product).

The poly(A) sequence as defined herein is suitably located at the 3′ terminus of the coding RNA. Accordingly it is preferred that the 3′-terminal nucleotide of the coding RNA (that is the last 3′-terminal nucleotide in the polynucleotide chain) is the 3′-terminal A nucleotide of the at least one poly(A) sequence. The term “located at the 3′ terminus” has to be understood as being located exactly at the 3′ terminus—in other words, the 3′ terminus of the coding RNA consists of a poly(A) sequence terminating with an A nucleotide. Sequences having a 3′ terminus consisting of a poly(A) sequence are SEQ ID NOs: 14152-14247, 14440-14535, 14728-14823, 15016-15111, 15336-15463, 15720-15847, 16104-16231, 16488-16615, 16846-16947, 17152-17253, 17458-17559, 17764-17865, 22124-22198, 22349-22423, 22574-22648, 22799-22873, 18206-18307, 18512-18613, 18818-18919, 19124-19225, 19566-19667, 19872-19973, 20178-20279, 20484-20585, 20927-21028, 21233-21334, 21539-21640, 21845-21946, 23237-23364, 23621-23748, 24005-24132, 24389-24516, 24933-25060, 25317-25444, 25701-25828, 26085-26212, 26629-26756, 27013-27140, 27397-27524, 27781-27908, 28325-28452, 28709-28836, 29093-29220, 29477-29604, 30021-30148, 30405-30532, 30789-30916, 31173-31300, 31717-31844, 32101-32228, 32485-32612, 32869-32996, 33413-33540, 33797-33924, 34181-34308, 34565-34692, 35109-35236, 35493-35620, 35877-36004, 36261-36388, 36805-36932, 37189-37316, 37573-37700, 37957-38084, 38501-38628, 38885-39012, 39269-39396, 39653-39780, 41893-42020, 42277-42404, 42661-42788, 43045-43172 (hSL-A100) or 14248-14343, 14536-14631, 14824-14919, 15112-15207, 15464-15591, 15848-15975, 16232-16359, 16616-16743, 16948-17049, 17254-17355, 17560-17661, 17866-17967, 22199-22273, 22424-22498, 22649-22723, 22874-22948, 18308-18409, 18614-18715, 18920-19021, 19226-19327, 19668-19769, 19974-20075, 20280-20381, 20586-20687, 21029-21130, 21335-21436, 21641-21742, 21947-22048, 23365-23492, 23749-23876, 24133-24260, 24517-24644, 25061-25188, 25445-25572, 25829-25956, 26213-26340, 26757-26884, 27141-27268, 27525-27652, 27909-28036, 28453-28580, 28837-28964, 29221-29348, 29605-29732, 30149-30276, 30533-30660, 30917-31044, 31301-31428, 31845-31972, 32229-32356, 32613-32740, 32997-33124, 33541-33668, 33925-34052, 34309-34436, 34693-34820, 35237-35364, 35621-35748, 36005-36132, 36389-36516, 36933-37060, 37317-37444, 37701-37828, 38085-38212, 38629-38756, 39013-39140, 39397-39524, 39781-39908, 42021-42148, 42405-42532, 42789-42916, 43173-43300 (A100) indicated under <223> identifier of the respective SEQ ID NO with “hSL-A100” or “A100” in the sequence listing.

The presence of a poly(A) sequence exactly at the 3′ terminus of the coding RNA encoding a LASV antigenic peptide protein (e.g. GPC) is advantageous and of particular importance in the context of the invention as the induction of a specific immune response against LASV antigenic peptide or protein can increase).

Preferably, the poly(A) sequence of the RNA is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a methods and means as described in WO2016/174271.

In embodiments, the RNA may comprise a poly(A) sequence derived from a template DNA and may comprise at least one additional poly(A) sequence generated by enzymatic polyadenylation, e.g. as described in WO2016/091391.

In preferred embodiments, the RNA may comprise at least one poly(C) sequence, preferably comprising 10 to 40 cytosine nucleotides.

In preferred embodiments, the poly(C) sequence, suitable located at the 3′ terminus (e.g. downstream of the 3′-UTR as defined herein), comprises 10 to 200 cytosine nucleotides, 10 to 100 cytosine nucleotides, 20 to 70 cytosine nucleotides, 20 to 60 cytosine nucleotides, or 10 to 40 cytosine nucleotides. In a particularly preferred embodiment, the poly(C) sequence comprises about 30 cytosine nucleotides.

The term “poly(C) sequence” as used herein will be recognized and understood by the person of ordinary skill in the art, and are for example intended to be a sequence of cytosine nucleotides, typically located at the 3′-end of an RNA, of up to about 200 cytosine nucleotides. In the context of the present invention, a poly(C) sequence may be located within an mRNA or any other nucleic acid molecule, such as in a DNA serving as template for the generation of an RNA, preferably an mRNA, e.g., by transcription said DNA template (e.g., plasmid DNA or PCR product).

Preferably, the poly(C) sequence in the RNA sequence of the present invention is derived from a DNA template by RNA in vitro transcription. In other embodiments, the poly(C) sequence is obtained in vitro by common methods of chemical synthesis without being necessarily transcribed from a DNA template.

In other embodiments, the RNA of the invention does not comprise a poly(C) sequence as defined herein.

In preferred embodiments, the coding RNA of the invention does comprise a poly(A) sequence as defined herein, preferably A100 located (exactly) at the 3′ terminus, and does not comprise a poly(C) sequence.

In a particularly preferred embodiment, the coding RNA of the invention comprises a cap1 structure as defined herein and at least one poly(A) sequence as defined in herein. Preferably, said cap1 structure is obtainable by co-transcriptional capping as defined herein, and said poly(A) sequence is preferably (exactly) at the 3′ terminus (e.g., A100, hSL-A100).

The presence of cap1 structure and poly(A) sequence exactly at the 3′ terminus of the coding RNA encoding a LASV peptide or protein (e.g. GPC) is advantageous as the induction of a specific immune response against LASV antigenic peptide or proteins may be dramatically increase.

In preferred embodiments, the RNA of the first aspect comprises at least one histone stem-loop.

The term “histone stem-loop” as used herein will be recognized and understood by the person of ordinary skill in the art, and is e.g. intended to refer to nucleic acid sequences that are predominantly found in histone mRNAs. Exemplary histone stem-loop sequences are described in Lopez et al. (Davila Lopez et al, (2008), RNA, 14(1)). The stem-loops in histone pre-mRNAs are typically followed by a purine-rich sequence known as the histone downstream element (HDE). These pre-mRNAs are processed in the nucleus by a single endonucleolytic cleavage approximately 5 nucleotides downstream of the stem-loop, catalysed by the U7 snRNP through base pairing of the U7 snRNA with the HDE.

Histone stem-loop sequences may suitably be selected from histone stem-loop sequences disclosed in WO2012/019780, the disclosure relating to histone stem-loop sequences/structures incorporated herewith by reference. A histone stem-loop sequence that may be used within the present invention may preferably be derived from formulae (I) or (II) of WO2012/019780. According to a further preferred embodiment the RNA as defined herein may comprise at least one histone stem-loop sequence derived from at least one of the specific formulae (Ia) or (IIa) of WO2012/019780.

In particularly preferred embodiment, the RNA of the invention comprises at least one histone stem-loop, wherein said histone stem-loop comprises a nucleic acid sequence according to SEQ ID NOs: 13842 or 13843 or a fragments or variant thereof.

In other embodiments, the RNA of the first aspect does not comprise a histone stem-loop as defined herein.

In further embodiments, the RNA of the invention comprises a 3′-terminal sequence element. Said 3′-terminal sequence element has to be understood as a sequence element comprising a poly(A) sequence and/or a histone-stem-loop sequence, wherein said sequence element is located at the 3′ terminus of the RNA of the invention.

Accordingly, the RNA of the invention may comprise a 3′-terminal sequence element according to SEQ ID NOs: 13848-13867, 13873-13879 or a fragment or variant thereof.

In preferred embodiments of the first aspect, the RNA, preferably mRNA comprises preferably in 5′- to 3′-direction the following elements:
a) 5′-cap structure, preferably as specified herein;
b) optionally, 5′-UTR as specified herein, preferably at least one selected from SEQ ID NOs: 13804-13825;
c) at least one coding sequence as specified herein;
d) 3′-UTR as specified herein, preferably at least one selected from SEQ ID NOs: 13826-13841;
e) optionally, poly(A) sequence, preferably as specified herein;
f) optionally, poly(C) sequence, preferably as specified herein;
g) optionally, histone stem-loop as specified herein, preferably according to SEQ ID NOs: 13842 or 13843;
h) optionally, 3′-terminal sequence element as specified herein, preferably according to SEQ ID NOs: 13848-13867, 13873-13879.

In further preferred embodiments of the first aspect, the RNA, preferably mRNA comprises the following elements preferably in 5′- to 3′-direction:
a) 5′-cap structure, preferably as specified herein, most preferably m7G(5′), m7G(5′)ppp(5′)(2′OMeA), or m7G(5′)ppp(5′)(2′OMeG);
b) 3′-UTR and 5′-UTR element according to a-1, a-2, a-3, a-4, a-5, b-1, b-2, b-3, b-4, b-5, c-1, c-2, c-3, c-4, c-5, d-1, d-2, d-3, d-4, d-5, e-1, e-2, e-3, e-4, e-5, e-6, f-1, f-2, f-3, f-4, f-5, g-1, g-2, g-3, g-4, g-5, h-1, h-2, h-3, h-4, h-5, i-1, i-2, or i-3, as specified herein, wherein a-1, a-2, b-4, i-2, or i-3 are preferred;
c) at least one coding sequence as specified herein, wherein said coding sequence is located between said 5′-UTR and said 3′-UTR, preferably downstream of said 5′-UTR and upstream of said 3′-UTR, wherein the coding sequence is preferably selected from any one specified in Table 2-5, columns “cds”;
d) optionally, poly(A) sequence, preferably as specified herein;
e) optionally, poly(C) sequence, preferably as specified herein;
f) optionally, histone stem-loop, preferably as specified herein;
g) optionally, 3′-terminal sequence element as specified herein, preferably according to according to SEQ ID NOs: 13848-13867, 13873-13879.

In further preferred embodiments of the first aspect, the RNA, preferably mRNA comprises the following elements in 5′- to 3′-direction:
a) 5′-cap structure, preferably as specified herein, most preferably m7G(5′), m7G(5′)ppp(5′)(2′OMeA), or m7G(5′)ppp(5′)(2′OMeG);
b) 3′-UTR and 5′-UTR element according to a-1, a-2, b-4, i-2, or i-3 as specified herein;
c) at least one coding sequence as specified herein, wherein said coding region is located between said 5′-UTR and said 3′-UTR, preferably downstream of said 5′-UTR and upstream of said 3′-UTR, wherein the coding sequence is preferably selected from any one of SEQ ID NOs: 255-2286, 3821-6106, 7798-9805, 11348-12803, 18002-18103, 19362-19463, 20723-20824, 22981-23108, 24677-24804, 26373-26500, 28069-28196, 29765-29892, 31461-31588, 33157-33284, 34853-34980, 36549-36676, 38245-38372, 41637-41764 (or fragments or variants thereof);
d) poly(A) sequence comprising about 64 adenosine;
e) poly(C) sequence comprising about 30 cysteines;
f) histone stem-loop according to SEQ ID NOs: 13842 or 13843.

In further preferred embodiments of the first aspect, the RNA, preferably mRNA comprises the following elements in 5′- to 3′-direction:
a) 5′-cap structure, preferably as specified herein, most preferably m7G(5′), m7G(5′)ppp(5′)(2′OMeA), or m7G(5′)ppp(5′)(2′OMeG);
b) 3′-UTR and 5′-UTR element according to a-1, a-2, b-4, i-2, or i-3 as specified herein;
c) at least one coding sequence as specified herein, wherein said coding region is located between said 5′-UTR and said 3′-UTR, preferably downstream of said 5′-UTR and upstream of said 3′-UTR, wherein the coding sequence is preferably selected from any one of SEQ ID NOs: 255-2286, 3821-6106, 7798-9805, 11348-12803, 18002-18103, 19362-19463, 20723-20824, 22981-23108, 24677-24804, 26373-26500, 28069-28196, 29765-29892, 31461-31588, 33157-33284, 34853-34980, 36549-36676, 38245-38372, 41637-41764 (or fragments or variants thereof);
d) 3′-terminal sequence element as specified herein, preferably according to according to SEQ ID NOs: 13848-13867, 13873-13879.

In further preferred embodiments of the first aspect, the RNA, preferably mRNA comprises the following elements in 5′- to 3′-direction:
a) 5′-cap structure, preferably as specified herein, most a cap1 structure;
b) 3′-UTR and 5′-UTR element according to a-1, a-2, b-4, i-2, or i-3 as specified herein;
c) at least one coding sequence as specified herein, wherein said coding region is located between said 5′-UTR and said 3′-UTR, preferably downstream of said 5′-UTR and upstream of said 3′-UTR, wherein the coding sequence is preferably selected from any one of SEQ ID NOs: 255-2286, 3821-6106, 7798-9805, 11348-12803, 18002-18103, 19362-19463, 20723-20824, 22981-23108, 24677-24804, 26373-26500, 28069-28196, 29765-29892, 31461-31588, 33157-33284, 34853-34980, 36549-36676, 38245-38372, 41637-41764 (or fragments or variants thereof);
d) a poly(A) sequence, preferably wherein the poly(A) sequence located (exactly) at the 3′ terminus,
e) 3′-terminal sequence element as specified herein, preferably according to according to SEQ ID NOs: 13848-13867, 13873-13879, and
wherein optionally at least one or more than one, preferably wherein all uracil nucleotides are replaced by pseudouridine (ψ) nucleotides or N1-methylpseudouridine (m1ψ) nucleotides.

Preferred amino acid sequences, nucleic acid sequences, and mRNA sequences of the invention are provided in Table 2 (GPC), Table 3.1 (GPCmut1, GPCstabilized), Table 3.2 (GPCmut2), Table 3.3 (GPCmut3), Table 3.4 (GPCmut4), Table 3.5 (GPCmut5), Table 3.6 (GPCmut6), Table 3.7 (GPCmut7), Table 3.8 (GPCmut8), Table 3.9 (GPCmut9), Table 3.10 (GPCmut10), Table 3.11 (GPCmut11), Table 3.12 (GPCmut12), Table 3.13 (GPCmut13), Table 4 (NP), Table 4.1 (SP-HsPLAT_NP), Table 4.2 (SP-HsALB_NP), Table 4.3 (SP-IgE_NP), and Table 5 (Z). Therein, each row represents a specific suitable LASV antigen of the invention, wherein the SEQ ID NOs of the amino acid sequence is provided in Column (“PRT”). The respective LASV strain where the amino acid sequence is derived from, NCBI accession number(s), and further information (e.g. clade) is provided under <223> identifier of the respective SEQ ID NO in the sequence listing.

The corresponding SEQ ID NOs of the coding sequences encoding the respective protein indicated in Column A is provided in column “CDS”. In said column, the coding sequences are provided in the following order: wild type coding sequence, opt1, opt2, opt3, opt4, opt5, opt11. Further information is provided under <223> identifier of the respective SEQ ID NO in the sequence listing.

The corresponding RNA sequences comprising preferred coding sequences are provided in column “mRNA designs and SEQ ID NOs”, wherein a-1 provides RNA sequences with advantageous UTR combination a-1 as defined herein, wherein a-2 provides mRNA sequences with advantageous UTR combination a-2 as defined herein, wherein b-4 provides mRNA sequences with advantageous UTR combination b-4 as defined herein, wherein i-2 provides mRNA sequences with advantageous UTR combination i-2 as defined herein, and wherein i-3 provides mRNA sequences with UTR combination i-3 as defined herein. Further information e.g. regarding the type of coding sequence (wild type, opt1, opt2, opt3, opt4, opt5, opt6, opt11) comprised in the RNA constructs are provided under <223> identifier of the respective SEQ ID NO in the sequence listing.

TABLE 2
Preferred LASV Glycoprotein (GPC) constructs

| | | RNA designs and SEQ ID NOs | | | | |
|---|---|---|---|---|---|---|
| PRT | CDS | a-1 | a-2 | b-4 | i-2 | i-3 |
| 1 | 255, | 2287, 2319, | 2543, 2575, | 2799, 2831, | 3055, | 3311, 3343, |
| | 509, | 2351, 2383, | 2607, 2639, | 2863, 2895, | 3087, | 3375, 3407, |
| | 763, | 2415, 2447, | 2671, 2703, | 2927, 2959, | 3119, | 3439, 3471, |
| | 1017, | 2479, 2511, | 2735, 2767, | 2991, 3023, | 3151, | 3503, 3535, |
| | 1271, | 14056, 14088, | 14344, 14376, | 14632, 14664, | 3183, | 14920, 14952, |
| | 1525, | 14120, 14152, | 14408, 14440, | 14696, 14728, | 3215, | 14984, 15016, |
| | 1779, | 14184, 14216, | 14472, 14504, | 14760, 14792, | 3247, | 15048, 15080, |
| | 2033 | 14248, 14280, | 14536, 14568, | 14824, 14856, | 3279 | 15112, 15144, |
| | | 14312 | 14600 | 14888 | | 15176 |
| 2 | 256, | 2288, 2320, | 2544, 2576, | 2800, 2832, | 3056, | 3312, 3344, |
| | 510, | 2352, 2384, | 2608, 2640, | 2864, 2896, | 3088, | 3376, 3408, |
| | 764, | 2416, 2448, | 2672, 2704, | 2928, 2960, | 3120, | 3440, 3472, |
| | 1018, | 2480, 2512, | 2736, 2768, | 2992, 3024, | 3152, | 3504, 3536, |
| | 1272, | 14057, 14089, | 14345, 14377, | 14633, 14665, | 3184, | 14921, 14953, |
| | 1526, | 14121, 14153, | 14409, 14441, | 14697, 14729, | 3216, | 14985, 15017, |
| | 1780, | 14185, 14217, | 14473, 14505, | 14761, 14793, | 3248, | 15049, 15081, |
| | 2034 | 14249, 14281, | 14537, 14569, | 14825, 14857, | 3280 | 15113, 15145, |
| | | 14313 | 14601 | 14889 | | 15177 |
| 3 | 257, | 2289, 2321, | 2545, 2577, | 2801, 2833, | 3057, | 3313, 3345, |
| | 511, | 2353, 2385, | 2609, 2641, | 2865, 2897, | 3089, | 3377, 3409, |
| | 765, | 2417, 2449, | 2673, 2705, | 2929, 2961, | 3121, | 3441, 3473, |
| | 1019, | 2481, 2513, | 2737, 2769, | 2993, 3025, | 3153, | 3505, 3537, |
| | 1273, | 14058, 14090, | 14346, 14378, | 14634, 14666, | 3185, | 14922, 14954, |
| | 1527, | 14122, 14154, | 14410, 14442, | 14698, 14730, | 3217, | 14986, 15018, |
| | 1781, | 14186, 14218, | 14474, 14506, | 14762, 14794, | 3249, | 15050, 15082, |
| | 2035 | 14250, 14282, | 14538, 14570, | 14826, 14858, | 3281 | 15114, 15146, |
| | | 14314 | 14602 | 14890 | | 15178 |
| 4 | 258, | 2290, 2322, | 2546, 2578, | 2802, 2834, | 3058, | 3314, 3346, |
| | 512, | 2354, 2386, | 2610, 2642, | 2866, 2898, | 3090, | 3378, 3410, |
| | 766, | 2418, 2450, | 2674, 2706, | 2930, 2962, | 3122, | 3442, 3474, |
| | 1020, | 2482, 2514, | 2738, 2770, | 2994, 3026, | 3154, | 3506, 3538, |
| | 1274, | 14059, 14091, | 14347, 14379, | 14635, 14667, | 3186, | 14923, 14955, |
| | 1528, | 14123, 14155, | 14411, 14443, | 14699, 14731, | 3218, | 14987, 15019, |
| | 1782, | 14187, 14219, | 14475, 14507, | 14763, 14795, | 3250, | 15051, 15083, |
| | 2036 | 14251, 14283, | 14539, 14571, | 14827, 14859, | 3282 | 15115, 15147, |
| | | 14315 | 14603 | 14891 | | 15179 |
| 5 | 259, | 2291, 2323, | 2547, 2579, | 2803, 2835, | 3059, | 3315, 3347, |
| | 513, | 2355, 2387, | 2611, 2643, | 2867, 2899, | 3091, | 3379, 3411, |
| | 767, | 2419, 2451, | 2675, 2707, | 2931, 2963, | 3123, | 3443, 3475, |
| | 1021, | 2483, 2515, | 2739, 2771, | 2995, 3027, | 3155, | 3507, 3539, |
| | 1275, | 14060, 14092, | 14348, 14380, | 14636, 14668, | 3187, | 14924, 14956, |
| | 1529, | 14124, 14156, | 14412, 14444, | 14700, 14732, | 3219, | 14988, 15020, |
| | 1783, | 14188, 14220, | 14476, 14508, | 14764, 14796, | 3251, | 15052, 15084, |
| | 2037 | 14252, 14284, | 14540, 14572, | 14828, 14860, | 3283 | 15116, 15148, |
| | | 14316 | 14604 | 14892 | | 15180 |
| 6 | 260, | 2292, 2324, | 2548, 2580, | 2804, 2836, | 3060, | 3316, 3348, |
| | 514, | 2356, 2388, | 2612, 2644, | 2868, 2900, | 3092, | 3380, 3412, |
| | 768, | 2420, 2452, | 2676, 2708, | 2932, 2964, | 3124, | 3444, 3476, |
| | 1022, | 2484, 2516, | 2740, 2772, | 2996, 3028, | 3156, | 3508, 3540, |
| | 1276, | 14061, 14093, | 14349, 14381, | 14637, 14669, | 3188, | 14925, 14957, |
| | 1530, | 14125, 14157, | 14413, 14445, | 14701, 14733, | 3220, | 14989, 15021, |
| | 1784, | 14189, 14221, | 14477, 14509, | 14765, 14797, | 3252, | 15053, 15085, |
| | 2038 | 14253, 14285, | 14541, 14573, | 14829, 14861, | 3284 | 15117, 15149, |
| | | 14317 | 14605 | 14893 | | 15181 |
| 7 | 261, | 2293, 2325, | 2549, 2581, | 2805, 2837, | 3061, | 3317, 3349, |
| | 515, | 2357, 2389, | 2613, 2645, | 2869, 2901, | 3093, | 3381, 3413, |
| | 769, | 2421, 2453, | 2677, 2709, | 2933, 2965, | 3125, | 3445, 3477, |
| | 1023, | 2485, 2517, | 2741, 2773, | 2997, 3029, | 3157, | 3509, 3541, |
| | 1277, | 14062, 14094, | 14350, 14382, | 14638, 14670, | 3189, | 14926, 14958, |
| | 1531, | 14126, 14158, | 14414, 14446, | 14702, 14734, | 3221, | 14990, 15022, |
| | 1785, | 14190, 14222, | 14478, 14510, | 14766, 14798, | 3253, | 15054, 15086, |
| | 2039 | 14254, 14286, | 14542, 14574, | 14830, 14862, | 3285 | 15118, 15150, |
| | | 14318 | 14606 | 14894 | | 15182 |
| 9 | 263, | 2294, 2326, | 2550, 2582, | 2806, 2838, | 3062, | 3318, 3350, |
| | 517, | 2358, 2390, | 2614, 2646, | 2870, 2902, | 3094, | 3382, 3414, |
| | 771, | 2422, 2454, | 2678, 2710, | 2934, 2966, | 3126, | 3446, 3478, |
| | 1025, | 2486, 2518, | 2742, 2774, | 2998, 3030, | 3158, | 3510, 3542, |
| | 1279, | 14063, 14095, | 14351, 14383, | 14639, 14671, | 3190, | 14927, 14959, |
| | 1533, | 14127, 14159, | 14415, 14447, | 14703, 14735, | 3222, | 14991, 15023, |
| | 1787, | 14191, 14223, | 14479, 14511, | 14767, 14799, | 3254, | 15055, 15087, |
| | 2041 | 14255, 14287, | 14543, 14575, | 14831, 14863, | 3286 | 15119, 15151, |
| | | 14319 | 14607 | 14895 | | 15183 |
| 10 | 264, | 2295, 2327, | 2551, 2583, | 2807, 2839, | 3063, | 3319, 3351, |
| | 518, | 2359, 2391, | 2615, 2647, | 2871, 2903, | 3095, | 3383, 3415, |
| | 772, | 2423, 2455, | 2679, 2711, | 2935, 2967, | 3127, | 3447, 3479, |
| | 1026, | 2487, 2519, | 2743, 2775, | 2999, 3031, | 3159, | 3511, 3543, |
| | 1280, | 14064, 14096, | 14352, 14384, | 14640, 14672, | 3191, | 14928, 14960, |
| | 1534, | 14128, 14160, | 14416, 14448, | 14704, 14736, | 3223, | 14992, 15024, |
| | 1788, | 14192, 14224, | 14480, 14512, | 14768, 14800, | 3255, | 15056, 15088, |
| | 2042 | 14256, 14288, | 14544, 14576, | 14832, 14864, | 3287 | 15120, 15152, |
| | | 14320 | 14608 | 14896 | | 15184 |
| 29 | 283, | 2296, 2328, | 2552, 2584, | 2808, 2840, | 3064, | 3320, 3352, |
| | 537, | 2360, 2392, | 2616, 2648, | 2872, 2904, | 3096, | 3384, 3416, |
| | 791, | 2424, 2456, | 2680, 2712, | 2936, 2968, | 3128, | 3448, 3480, |
| | 1045, | 2488, 2520, | 2744, 2776, | 3000, 3032, | 3160, | 3512, 3544, |
| | 1299, | 14065, 14097, | 14353, 14385, | 14641, 14673, | 3192, | 14929, 14961, |
| | 1553, | 14129, 14161, | 14417, 14449, | 14705, 14737, | 3224, | 14993, 15025, |
| | 1807, | 14193, 14225, | 14481, 14513, | 14769, 14801, | 3256, | 15057, 15089, |
| | 2061 | 14257, 14289, | 14545, 14577, | 14833, 14865, | 3288 | 15121, 15153, |
| | | 14321 | 14609 | 14897 | | 15185 |
| 50 | 304, | 2297, 2329, | 2553, 2585, | 2809, 2841, | 3065, | 3321, 3353, |
| | 558, | 2361, 2393, | 2617, 2649, | 2873, 2905, | 3097, | 3385, 3417, |
| | 812, | 2425, 2457, | 2681, 2713, | 2937, 2969, | 3129, | 3449, 3481, |
| | 1066, | 2489, 2521, | 2745, 2777, | 3001, 3033, | 3161, | 3513, 3545, |
| | 1320, | 14066, 14098, | 14354, 14386, | 14642, 14674, | 3193, | 14930, 14962, |
| | 1574, | 14130, 14162, | 14418, 14450, | 14706, 14738, | 3225, | 14994, 15026, |
| | 1828, | 14194, 14226, | 14482, 14514, | 14770, 14802, | 3257, | 15058, 15090, |
| | 2082 | 14258, 14290, | 14546, 14578, | 14834, 14866, | 3289 | 15122, 15154, |
| | | 14322 | 14610 | 14898 | | 15186 |
| 60 | 314, | 2298, 2330, | 2554, 2586, | 2810, 2842, | 3066, | 3322, 3354, |
| | 568, | 2362, 2394, | 2618, 2650, | 2874, 2906, | 3098, | 3386, 3418, |
| | 822, | 2426, 2458, | 2682, 2714, | 2938, 2970, | 3130, | 3450, 3482, |
| | 1076, | 2490, 2522, | 2746, 2778, | 3002, 3034, | 3162, | 3514, 3546, |
| | 1330, | 14067, 14099, | 14355, 14387, | 14643, 14675, | 3194, | 14931, 14963, |
| | 1584, | 14131, 14163, | 14419, 14451, | 14707, 14739, | 3226, | 14995, 15027, |
| | 1838, | 14195, 14227, | 14483, 14515, | 14771, 14803, | 3258, | 15059, 15091, |
| | 2092 | 14259, 14291, | 14547, 14579, | 14835, 14867, | 3290 | 15123, 15155, |
| | | 14323 | 14611 | 14899 | | 15187 |
| 61 | 315, | 2299, 2331, | 2555, 2587, | 2811, 2843, | 3067, | 3323, 3355, |
| | 569, | 2363, 2395, | 2619, 2651, | 2875, 2907, | 3099, | 3387, 3419, |
| | 823, | 2427, 2459, | 2683, 2715, | 2939, 2971, | 3131, | 3451, 3483, |
| | 1077, | 2491, 2523, | 2747, 2779, | 3003, 3035, | 3163, | 3515, 3547, |
| | 1331, | 14068, 14100, | 14356, 14388, | 14644, 14676, | 3195, | 14932, 14964, |
| | 1585, | 14132, 14164, | 14420, 14452, | 14708, 14740, | 3227, | 14996, 15028, |
| | 1839, | 14196, 14228, | 14484, 14516, | 14772, 14804, | 3259, | 15060, 15092, |
| | 2093 | 14260, 14292, | 14548, 14580, | 14836, 14868, | 3291 | 15124, 15156, |
| | | 14324 | 14612 | 14900 | | 15188 |
| 105 | 359, | 2300, 2332, | 2556, 2588, | 2812, 2844, | 3068, | 3324, 3356, |
| | 613, | 2364, 2396, | 2620, 2652, | 2876, 2908, | 3100, | 3388, 3420, |
| | 867, | 2428, 2460, | 2684, 2716, | 2940, 2972, | 3132, | 3452, 3484, |
| | 1121, | 2492, 2524, | 2748, 2780, | 3004, 3036, | 3164, | 3516, 3548, |
| | 1375, | 14069, 14101, | 14357, 14389, | 14645, 14677, | 3196, | 14933, 14965, |
| | 1629, | 14133, 14165, | 14421, 14453, | 14709, 14741, | 3228, | 14997, 15029, |
| | 1883, | 14197, 14229, | 14485, 14517, | 14773, 14805, | 3260, | 15061, 15093, |
| | 2137 | 14261, 14293, | 14549, 14581, | 14837, 14869, | 3292 | 15125, 15157, |
| | | 14325 | 14613 | 14901 | | 15189 |
| 112 | 366, | 2301, 2333, | 2557, 2589, | 2813, 2845, | 3069, | 3325, 3357, |
| | 620, | 2365, 2397, | 2621, 2653, | 2877, 2909, | 3101, | 3389, 3421, |
| | 874, | 2429, 2461, | 2685, 2717, | 2941, 2973, | 3133, | 3453, 3485, |
| | 1128, | 2493, 2525, | 2749, 2781, | 3005, 3037, | 3165, | 3517, 3549, |
| | 1382, | 14070, 14102, | 14358, 14390, | 14646, 14678, | 3197, | 14934, 14966, |
| | 1636, | 14134, 14166, | 14422, 14454, | 14710, 14742, | 3229, | 14998, 15030, |
| | 1890, | 14198, 14230, | 14486, 14518, | 14774, 14806, | 3261, | 15062, 15094, |
| | 2144 | 14262, 14294, | 14550, 14582, | 14838, 14870, | 3293 | 15126, 15158, |
| | | 14326 | 14614 | 14902 | | 15190 |
| 114 | 368, | 2302, 2334, | 2558, 2590, | 2814, 2846, | 3070, | 3326, 3358, |
| | 622, | 2366, 2398, | 2622, 2654, | 2878, 2910, | 3102, | 3390, 3422, |
| | 876, | 2430, 2462, | 2686, 2718, | 2942, 2974, | 3134, | 3454, 3486, |
| | 1130, | 2494, 2526, | 2750, 2782, | 3006, 3038, | 3166, | 3518, 3550, |
| | 1384, | 14071, 14103, | 14359, 14391, | 14647, 14679, | 3198, | 14935, 14967, |
| | 1638, | 14135, 14167, | 14423, 14455, | 14711, 14743, | 3230, | 14999, 15031, |
| | 1892, | 14199, 14231, | 14487, 14519, | 14775, 14807, | 3262, | 15063, 15095, |
| | 2146 | 14263, 14295, | 14551, 14583, | 14839, 14871, | 3294 | 15127, 15159, |
| | | 14327 | 14615 | 14903 | | 15191 |
| 123 | 377, | 2303, 2335, | 2559, 2591, | 2815, 2847, | 3071, | 3327, 3359, |
| | 631, | 2367, 2399, | 2623, 2655, | 2879, 2911, | 3103, | 3391, 3423, |
| | 885, | 2431, 2463, | 2687, 2719, | 2943, 2975, | 3135, | 3455, 3487, |
| | 1139, | 2495, 2527, | 2751, 2783, | 3007, 3039, | 3167, | 3519, 3551, |
| | 1393, | 14072, 14104, | 14360, 14392, | 14648, 14680, | 3199, | 14936, 14968, |
| | 1647, | 14136, 14168, | 14424, 14456, | 14712, 14744, | 3231, | 15000, 15032, |
| | 1901, | 14200, 14232, | 14488, 14520, | 14776, 14808, | 3263, | 15064, 15096, |
| | 2155 | 14264, 14296, | 14552, 14584, | 14840, 14872, | 3295 | 15128, 15160, |
| | | 14328 | 14616 | 14904 | | 15192 |
| 127 | 381, | 2304, 2336, | 2560, 2592, | 2816, 2848, | 3072, | 3328, 3360, |
| | 635, | 2368, 2400, | 2624, 2656, | 2880, 2912, | 3104, | 3392, 3424, |
| | 889, | 2432, 2464, | 2688, 2720, | 2944, 2976, | 3136, | 3456, 3488, |
| | 1143, | 2496, 2528, | 2752, 2784, | 3008, 3040, | 3168, | 3520, 3552, |
| | 1397, | 14073, 14105, | 14361, 14393, | 14649, 14681, | 3200, | 14937, 14969, |
| | 1651, | 14137, 14169, | 14425, 14457, | 14713, 14745, | 3232, | 15001, 15033, |
| | 1905, | 14201, 14233, | 14489, 14521, | 14777, 14809, | 3264, | 15065, 15097, |
| | 2159 | 14265, 14297, | 14553, 14585, | 14841, 14873, | 3296 | 15129, 15161, |
| | | 14329 | 14617 | 14905 | | 15193 |
| 131 | 385, | 2305, 2337, | 2561, 2593, | 2817, 2849, | 3073, | 3329, 3361, |
| | 639, | 2369, 2401, | 2625, 2657, | 2881, 2913, | 3105, | 3393, 3425, |
| | 893, | 2433, 2465, | 2689, 2721, | 2945, 2977, | 3137, | 3457, 3489, |
| | 1147, | 2497, 2529, | 2753, 2785, | 3009, 3041, | 3169, | 3521, 3553, |
| | 1401, | 14074, 14106, | 14362, 14394, | 14650, 14682, | 3201, | 14938, 14970, |
| | 1655, | 14138, 14170, | 14426, 14458, | 14714, 14746, | 3233, | 15002, 15034, |
| | 1909, | 14202, 14234, | 14490, 14522, | 14778, 14810, | 3265, | 15066, 15098, |
| | 2163 | 14266, 14298, | 14554, 14586, | 14842, 14874, | 3297 | 15130, 15162, |
| | | 14330 | 14618 | 14906 | | 15194 |
| 137 | 391, | 2306, 2338, | 2562, 2594, | 2818, 2850, | 3074, | 3330, 3362, |
| | 645, | 2370, 2402, | 2626, 2658, | 2882, 2914, | 3106, | 3394, 3426, |
| | 899, | 2434, 2466, | 2690, 2722, | 2946, 2978, | 3138, | 3458, 3490, |
| | 1153, | 2498, 2530, | 2754, 2786, | 3010, 3042, | 3170, | 3522, 3554, |
| | 1407, | 14075, 14107, | 14363, 14395, | 14651, 14683, | 3202, | 14939, 14971, |
| | 1661, | 14139, 14171, | 14427, 14459, | 14715, 14747, | 3234, | 15003, 15035, |
| | 1915, | 14203, 14235, | 14491, 14523, | 14779, 14811, | 3266, | 15067, 15099, |
| | 2169 | 14267, 14299, | 14555, 14587, | 14843, 14875, | 3298 | 15131, 15163, |
| | | 14331 | 14619 | 14907 | | 15195 |
| 138 | 392, | 2307, 2339, | 2563, 2595, | 2819, 2851, | 3075, | 3331, 3363, |
| | 646, | 2371, 2403, | 2627, 2659, | 2883, 2915, | 3107, | 3395, 3427, |
| | 900, | 2435, 2467, | 2691, 2723, | 2947, 2979, | 3139, | 3459, 3491, |
| | 1154, | 2499, 2531, | 2755, 2787, | 3011, 3043, | 3171, | 3523, 3555, |
| | 1408, | 14076, 14108, | 14364, 14396, | 14652, 14684, | 3203, | 14940, 14972, |
| | 1662, | 14140, 14172, | 14428, 14460, | 14716, 14748, | 3235, | 15004, 15036, |
| | 1916, | 14204, 14236, | 14492, 14524, | 14780, 14812, | 3267, | 15068, 15100, |
| | 2170 | 14268, 14300, | 14556, 14588, | 14844, 14876, | 3299 | 15132, 15164, |
| | | 14332 | 14620 | 14908 | | 15196 |
| 162 | 416, | 2308, 2340, | 2564, 2596, | 2820, 2852, | 3076, | 3332, 3364, |
| | 670, | 2372, 2404, | 2628, 2660, | 2884, 2916, | 3108, | 3396, 3428, |
| | 924, | 2436, 2468, | 2692, 2724, | 2948, 2980, | 3140, | 3460, 3492, |
| | 1178, | 2500, 2532, | 2756, 2788, | 3012, 3044, | 3172, | 3524, 3556, |
| | 1432, | 14077, 14109, | 14365, 14397, | 14653, 14685, | 3204, | 14941, 14973, |
| | 1686, | 14141, 14173, | 14429, 14461, | 14717, 14749, | 3236, | 15005, 15037, |
| | 1940, | 14205, 14237, | 14493, 14525, | 14781, 14813, | 3268, | 15069, 15101, |
| | 2194 | 14269, 14301, | 14557, 14589, | 14845, 14877, | 3300 | 15133, 15165, |
| | | 14333 | 14621 | 14909 | | 15197 |
| 167 | 421, | 2309, 2341, | 2565, 2597, | 2821, 2853, | 3077, | 3333, 3365, |
| | 675, | 2373, 2405, | 2629, 2661, | 2885, 2917, | 3109, | 3397, 3429, |
| | 929, | 2437, 2469, | 2693, 2725, | 2949, 2981, | 3141, | 3461, 3493, |
| | 1183, | 2501, 2533, | 2757, 2789, | 3013, 3045, | 3173, | 3525, 3557, |
| | 1437, | 14078, 14110, | 14366, 14398, | 14654, 14686, | 3205, | 14942, 14974, |
| | 1691, | 14142, 14174, | 14430, 14462, | 14718, 14750, | 3237, | 15006, 15038, |
| | 1945, | 14206, 14238, | 14494, 14526, | 14782, 14814, | 3269, | 15070, 15102, |
| | 2199 | 14270, 14302, | 14558, 14590, | 14846, 14878, | 3301 | 15134, 15166, |
| | | 14334 | 14622 | 14910 | | 15198 |
| 170 | 424, | 2310, 2342, | 2566, 2598, | 2822, 2854, | 3078, | 3334, 3366, |
| | 678, | 2374, 2406, | 2630, 2662, | 2886, 2918, | 3110, | 3398, 3430, |
| | 932, | 2438, 2470, | 2694, 2726, | 2950, 2982, | 3142, | 3462, 3494, |
| | 1186, | 2502, 2534, | 2758, 2790, | 3014, 3046, | 3174, | 3526, 3558, |
| | 1440, | 14079, 14111, | 14367, 14399, | 14655, 14687, | 3206, | 14943, 14975, |
| | 1694, | 14143, 14175, | 14431, 14463, | 14719, 14751, | 3238, | 15007, 15039, |
| | 1948, | 14207, 14239, | 14495, 14527, | 14783, 14815, | 3270, | 15071, 15103, |
| | 2202 | 14271, 14303, | 14559, 14591, | 14847, 14879, | 3302 | 15135, 15167, |
| | | 14335 | 14623 | 14911 | | 15199 |
| 209 | 463, | 2311, 2343, | 2567, 2599, | 2823, 2855, | 3079, | 3335, 3367, |
| | 717, | 2375, 2407, | 2631, 2663, | 2887, 2919, | 3111, | 3399, 3431, |
| | 971, | 2439, 2471, | 2695, 2727, | 2951, 2983, | 3143, | 3463, 3495, |
| | 1225, | 2503, 2535, | 2759, 2791, | 3015, 3047, | 3175, | 3527, 3559, |
| | 1479, | 14080, 14112, | 14368, 14400, | 14656, 14688, | 3207, | 14944, 14976, |
| | 1733, | 14144, 14176, | 14432, 14464, | 14720, 14752, | 3239, | 15008, 15040, |
| | 1987, | 14208, 14240, | 14496, 14528, | 14784, 14816, | 3271, | 15072, 15104, |
| | 2241 | 14272, 14304, | 14560, 14592, | 14848, 14880, | 3303 | 15136, 15168, |
| | | 14336 | 14624 | 14912 | | 15200 |
| 219 | 473, | 2312, 2344, | 2568, 2600, | 2824, 2856, | 3080, | 3336, 3368, |
| | 727, | 2376, 2408, | 2632, 2664, | 2888, 2920, | 3112, | 3400, 3432, |
| | 981, | 2440, 2472, | 2696, 2728, | 2952, 2984, | 3144, | 3464, 3496, |
| | 1235, | 2504, 2536, | 2760, 2792, | 3016, 3048, | 3176, | 3528, 3560, |
| | 1489, | 14081, 14113, | 14369, 14401, | 14657, 14689, | 3208, | 14945, 14977, |
| | 1743, | 14145, 14177, | 14433, 14465, | 14721, 14753, | 3240, | 15009, 15041, |
| | 1997, | 14209, 14241, | 14497, 14529, | 14785, 14817, | 3272, | 15073, 15105, |
| | 2251 | 14273, 14305, | 14561, 14593, | 14849, 14881, | 3304 | 15137, 15169, |
| | | 14337 | 14625 | 14913 | | 15201 |
| 224 | 478, | 2313, 2345, | 2569, 2601, | 2825, 2857, | 3081, | 3337, 3369, |
| | 732, | 2377, 2409, | 2633, 2665, | 2889, 2921, | 3113, | 3401, 3433, |
| | 986, | 2441, 2473, | 2697, 2729, | 2953, 2985, | 3145, | 3465, 3497, |
| | 1240, | 2505, 2537, | 2761, 2793, | 3017, 3049, | 3177, | 3529, 3561, |
| | 1494, | 14082, 14114, | 14370, 14402, | 14658, 14690, | 3209, | 14946, 14978, |
| | 1748, | 14146, 14178, | 14434, 14466, | 14722, 14754, | 3241, | 15010, 15042, |
| | 2002, | 14210, 14242, | 14498, 14530, | 14786, 14818, | 3273, | 15074, 15106, |
| | 2256 | 14274, 14306, | 14562, 14594, | 14850, 14882, | 3305 | 15138, 15170, |
| | | 14338 | 14626 | 14914 | | 15202 |
| 229 | 483, | 2314, 2346, | 2570, 2602, | 2826, 2858, | 3082, | 3338, 3370, |
| | 737, | 2378, 2410, | 2634, 2666, | 2890, 2922, | 3114, | 3402, 3434, |
| | 991, | 2442, 2474, | 2698, 2730, | 2954, 2986, | 3146, | 3466, 3498, |
| | 1245, | 2506, 2538, | 2762, 2794, | 3018, 3050, | 3178, | 3530, 3562, |
| | 1499, | 14083, 14115, | 14371, 14403, | 14659, 14691, | 3210, | 14947, 14979, |
| | 1753, | 14147, 14179, | 14435, 14467, | 14723, 14755, | 3242, | 15011, 15043, |
| | 2007, | 14211, 14243, | 14499, 14531, | 14787, 14819, | 3274, | 15075, 15107, |
| | 2261 | 14275, 14307, | 14563, 14595, | 14851, 14883, | 3306 | 15139, 15171, |
| | | 14339 | 14627 | 14915 | | 15203 |
| 235 | 489, | 2315, 2347, | 2571, 2603, | 2827, 2859, | 3083, | 3339, 3371, |
| | 743, | 2379, 2411, | 2635, 2667, | 2891, 2923, | 3115, | 3403, 3435, |
| | 997, | 2443, 2475, | 2699, 2731, | 2955, 2987, | 3147, | 3467, 3499, |
| | 1251, | 2507, 2539, | 2763, 2795, | 3019, 3051, | 3179, | 3531, 3563, |
| | 1505, | 14084, 14116, | 14372, 14404, | 14660, 14692, | 3211, | 14948, 14980, |
| | 1759, | 14148, 14180, | 14436, 14468, | 14724, 14756, | 3243, | 15012, 15044, |
| | 2013, | 14212, 14244, | 14500, 14532, | 14788, 14820, | 3275, | 15076, 15108, |
| | 2267 | 14276, 14308, | 14564, 14596, | 14852, 14884, | 3307 | 15140, 15172, |
| | | 14340 | 14628 | 14916 | | 15204 |
| 237 | 491, | 2316, 2348, | 2572, 2604, | 2828, 2860, | 3084, | 3340, 3372, |
| | 745, | 2380, 2412, | 2636, 2668, | 2892, 2924, | 3116, | 3404, 3436, |
| | 999, | 2444, 2476, | 2700, 2732, | 2956, 2988, | 3148, | 3468, 3500, |
| | 1253, | 2508, 2540, | 2764, 2796, | 3020, 3052, | 3180, | 3532, 3564, |
| | 1507, | 14085, 14117, | 14373, 14405, | 14661, 14693, | 3212, | 14949, 14981, |
| | 1761, | 14149, 14181, | 14437, 14469, | 14725, 14757, | 3244, | 15013, 15045, |
| | 2015, | 14213, 14245, | 14501, 14533, | 14789, 14821, | 3276, | 15077, 15109, |
| | 2269 | 14277, 14309, | 14565, 14597, | 14853, 14885, | 3308 | 15141, 15173, |
| | | 14341 | 14629 | 14917 | | 15205 |
| 239 | 493, | 2317, 2349, | 2573, 2605, | 2829, 2861, | 3085, | 3341, 3373, |
| | 747, | 2381, 2413, | 2637, 2669, | 2893, 2925, | 3117, | 3405, 3437, |
| | 1001, | 2445, 2477, | 2701, 2733, | 2957, 2989, | 3149, | 3469, 3501, |
| | 1255, | 2509, 2541, | 2765, 2797, | 3021, 3053, | 3181, | 3533, 3565, |
| | 1509, | 14086, 14118, | 14374, 14406, | 14662, 14694, | 3213, | 14950, 14982, |
| | 1763, | 14150, 14182, | 14438, 14470, | 14726, 14758, | 3245, | 15014, 15046, |
| | 2017, | 14214, 14246, | 14502, 14534, | 14790, 14822, | 3277, | 15078, 15110, |
| | 2271 | 14278, 14310, | 14566, 14598, | 14854, 14886, | 3309 | 15142, 15174, |
| | | 14342 | 14630 | 14918 | | 15206 |
| 245 | 499, | 2318, 2350, | 2574, 2606, | 2830, 2862, | 3086, | 3342, 3374, |
| | 753, | 2382, 2414, | 2638, 2670, | 2894, 2926, | 3118, | 3406, 3438, |
| | 1007, | 2446, 2478, | 2702, 2734, | 2958, 2990, | 3150, | 3470, 3502, |
| | 1261, | 2510, 2542, | 2766, 2798, | 3022, 3054, | 3182, | 3534, 3566, |
| | 1515, | 14087, 14119, | 14375, 14407, | 14663, 14695, | 3214, | 14951, 14983, |
| | 1769, | 14151, 14183, | 14439, 14471, | 14727, 14759, | 3246, | 15015, 15047, |
| | 2023, | 14215, 14247, | 14503, 14535, | 14791, 14823, | 3278, | 15079, 15111, |
| | 2277 | 14279, 14311, | 14567, 14599, | 14855, 14887, | 3310 | 15143, 15175, |
| | | 14343 | 14631 | 14919 | | 15207 |

TABLE 3.1
Preferred LASV prefusion-stabilized (GPCmut1) constructs

| | | RNA designs and SEQ ID NOs | | | | |
|---|---|---|---|---|---|---|
| PRT | CDS | a-1 | a-2 | b-4 | i-2 | i-3 |
| 3567 | 3821, | 6107, 6139, | 6395, 6427, | 6683, 6715, | 6971, | 7259, 7291, |
| | 4075, | 6171, 6203, | 6459, 6491, | 6747, 6779, | 7003, | 7323, 7355, |
| | 4329, | 6235, 6267, | 6523, 6555, | 6811, 6843, | 7035, | 7387, 7419, |
| | 4583, | 6299, 6331, | 6587, 6619, | 6875, 6907, | 7067, | 7451, 7483, |
| | 4837, | 6363, 15208, | 6651, 15592, | 6939, 15976, | 7099, | 7515, 16360, |
| | 5091, | 15240, 15272, | 15624, 15656, | 16008, 16040, | 7131, | 16392, 16424, |
| | 5345, | 15304, 15336, | 15688, 15720, | 16072, 16104, | 7163, | 16456, 16488, |
| | 5599, | 15368, 15400, | 15752, 15784, | 16136, 16168, | 7195, | 16520, 16552, |
| | 5853 | 15432, 15464, | 15816, 15848, | 16200, 16232, | 7227 | 16584, 16616, |
| | | 15496, 15528, | 15880, 15912, | 16264, 16296, | | 16648, 16680, |
| | | 15560 | 15944 | 16328 | | 16712 |
| 3568 | 3822, | 6108, 6140, | 6396, 6428, | 6684, 6716, | 6972, | 7260, 7292, |
| | 4076, | 6172, 6204, | 6460, 6492, | 6748, 6780, | 7004, | 7324, 7356, |
| | 4330, | 6236, 6268, | 6524, 6556, | 6812, 6844, | 7036, | 7388, 7420, |
| | 4584, | 6300, 6332, | 6588, 6620, | 6876, 6908, | 7068, | 7452, 7484, |
| | 4838, | 6364, 15209, | 6652, 15593, | 6940, 15977, | 7100, | 7516, 16361, |
| | 5092, | 15241, 15273, | 15625, 15657, | 16009, 16041, | 7132, | 16393, 16425, |
| | 5346, | 15305, 15337, | 15689, 15721, | 16073, 16105, | 7164, | 16457, 16489, |
| | 5600, | 15369, 15401, | 15753, 15785, | 16137, 16169, | 7196, | 16521, 16553, |
| | 5854 | 15433, 15465, | 15817, 15849, | 16201, 16233, | 7228 | 16585, 16617, |
| | | 15497, 15529, | 15881, 15913, | 16265, 16297, | | 16649, 16681, |
| | | 15561 | 15945 | 16329 | | 16713 |
| 3569 | 3823, | 6109, 6141, | 6397, 6429, | 6685, 6717, | 6973, | 7261, 7293, |
| | 4077, | 6173, 6205, | 6461, 6493, | 6749, 6781, | 7005, | 7325, 7357, |
| | 4331, | 6237, 6269, | 6525, 6557, | 6813, 6845, | 7037, | 7389, 7421, |
| | 4585, | 6301, 6333, | 6589, 6621, | 6877, 6909, | 7069, | 7453, 7485, |
| | 4839, | 6365, 15210, | 6653, 15594, | 6941, 15978, | 7101, | 7517, 16362, |
| | 5093, | 15242, 15274, | 15626, 15658, | 16010, 16042, | 7133, | 16394, 16426, |
| | 5347, | 15306, 15338, | 15690, 15722, | 16074, 16106, | 7165, | 16458, 16490, |
| | 5601, | 15370, 15402, | 15754, 15786, | 16138, 16170, | 7197, | 16522, 16554, |
| | 5855 | 15434, 15466, | 15818, 15850, | 16202, 16234, | 7229 | 16586, 16618, |
| | | 15498, 15530, | 15882, 15914, | 16266, 16298, | | 16650, 16682, |
| | | 15562 | 15946 | 16330 | | 16714 |
| 3570 | 3824, | 6110, 6142, | 6398, 6430, | 6686, 6718, | 6974, | 7262, 7294, |
| | 4078, | 6174, 6206, | 6462, 6494, | 6750, 6782, | 7006, | 7326, 7358, |
| | 4332, | 6238, 6270, | 6526, 6558, | 6814, 6846, | 7038, | 7390, 7422, |
| | 4586, | 6302, 6334, | 6590, 6622, | 6878, 6910, | 7070, | 7454, 7486, |
| | 4840, | 6366, 15211, | 6654, 15595, | 6942, 15979, | 7102, | 7518, 16363, |
| | 5094, | 15243, 15275, | 15627, 15659, | 16011, 16043, | 7134, | 16395, 16427, |
| | 5348, | 15307, 15339, | 15691, 15723, | 16075, 16107, | 7166, | 16459, 16491, |
| | 5602, | 15371, 15403, | 15755, 15787, | 16139, 16171, | 7198, | 16523, 16555, |
| | 5856 | 15435, 15467, | 15819, 15851, | 16203, 16235, | 7230 | 16587, 16619, |
| | | 15499, 15531, | 15883, 15915, | 16267, 16299, | | 16651, 16683, |
| | | 15563 | 15947 | 16331 | | 16715 |
| 3571 | 3825, | 6111, 6143, | 6399, 6431, | 6687, 6719, | 6975, | 7263, 7295, |
| | 4079, | 6175, 6207, | 6463, 6495, | 6751, 6783, | 7007, | 7327, 7359, |
| | 4333, | 6239, 6271, | 6527, 6559, | 6815, 6847, | 7039, | 7391, 7423, |
| | 4587, | 6303, 6335, | 6591, 6623, | 6879, 6911, | 7071, | 7455, 7487, |
| | 4841, | 6367, 15212, | 6655, 15596, | 6943, 15980, | 7103, | 7519, 16364, |
| | 5095, | 15244, 15276, | 15628, 15660, | 16012, 16044, | 7135, | 16396, 16428, |
| | 5349, | 15308, 15340, | 15692, 15724, | 16076, 16108, | 7167, | 16460, 16492, |
| | 5603, | 15372, 15404, | 15756, 15788, | 16140, 16172, | 7199, | 16524, 16556, |
| | 5857 | 15436, 15468, | 15820, 15852, | 16204, 16236, | 7231 | 16588, 16620, |
| | | 15500, 15532, | 15884, 15916, | 16268, 16300, | | 16652, 16684, |
| | | 15564 | 15948 | 16332 | | 16716 |
| 3572 | 3826, | 6112, 6144, | 6400, 6432, | 6688, 6720, | 6976, | 7264, 7296, |
| | 4080, | 6176, 6208, | 6464, 6496, | 6752, 6784, | 7008, | 7328, 7360, |
| | 4334, | 6240, 6272, | 6528, 6560, | 6816, 6848, | 7040, | 7392, 7424, |
| | 4588, | 6304, 6336, | 6592, 6624, | 6880, 6912, | 7072, | 7456, 7488, |
| | 4842, | 6368, 15213, | 6656, 15597, | 6944, 15981, | 7104, | 7520, 16365, |
| | 5096, | 15245, 15277, | 15629, 15661, | 16013, 16045, | 7136, | 16397, 16429, |
| | 5350, | 15309, 15341, | 15693, 15725, | 16077, 16109, | 7168, | 16461, 16493, |
| | 5604, | 15373, 15405, | 15757, 15789, | 16141, 16173, | 7200, | 16525, 16557, |
| | 5858 | 15437, 15469, | 15821, 15853, | 16205, 16237, | 7232 | 16589, 16621, |
| | | 15501, 15533, | 15885, 15917, | 16269, 16301, | | 16653, 16685, |
| | | 15565 | 15949 | 16333 | | 16717 |
| 3573 | 3827, | 6113, 6145, | 6401, 6433, | 6689, 6721, | 6977, | 7265, 7297, |
| | 4081, | 6177, 6209, | 6465, 6497, | 6753, 6785, | 7009, | 7329, 7361, |
| | 4335, | 6241, 6273, | 6529, 6561, | 6817, 6849, | 7041, | 7393, 7425, |
| | 4589, | 6305, 6337, | 6593, 6625, | 6881, 6913, | 7073, | 7457, 7489, |
| | 4843, | 6369, 15214, | 6657, 15598, | 6945, 15982, | 7105, | 7521, 16366, |
| | 5097, | 15246, 15278, | 15630, 15662, | 16014, 16046, | 7137, | 16398, 16430, |
| | 5351, | 15310, 15342, | 15694, 15726, | 16078, 16110, | 7169, | 16462, 16494, |
| | 5605, | 15374, 15406, | 15758, 15790, | 16142, 16174, | 7201, | 16526, 16558, |
| | 5859 | 15438, 15470, | 15822, 15854, | 16206, 16238, | 7233 | 16590, 16622, |
| | | 15502, 15534, | 15886, 15918, | 16270, 16302, | | 16654, 16686, |
| | | 15566 | 15950 | 16334 | | 16718 |
| 3575 | 3829, | 6114, 6146, | 6402, 6434, | 6690, 6722, | 6978, | 7266, 7298, |
| | 4083, | 6178, 6210, | 6466, 6498, | 6754, 6786, | 7010, | 7330, 7362, |
| | 4337, | 6242, 6274, | 6530, 6562, | 6818, 6850, | 7042, | 7394, 7426, |
| | 4591, | 6306, 6338, | 6594, 6626, | 6882, 6914, | 7074, | 7458, 7490, |
| | 4845, | 6370, 15215, | 6658, 15599, | 6946, 15983, | 7106, | 7522, 16367, |
| | 5099, | 15247, 15279, | 15631, 15663, | 16015, 16047, | 7138, | 16399, 16431, |
| | 5353, | 15311, 15343, | 15695, 15727, | 16079, 16111, | 7170, | 16463, 16495, |
| | 5607, | 15375, 15407, | 15759, 15791, | 16143, 16175, | 7202, | 16527, 16559, |
| | 5861 | 15439, 15471, | 15823, 15855, | 16207, 16239, | 7234 | 16591, 16623, |
| | | 15503, 15535, | 15887, 15919, | 16271, 16303, | | 16655, 16687, |
| | | 15567 | 15951 | 16335 | | 16719 |
| 3576 | 3830, | 6115, 6147, | 6403, 6435, | 6691, 6723, | 6979, | 7267, 7299, |
| | 4084, | 6179, 6211, | 6467, 6499, | 6755, 6787, | 7011, | 7331, 7363, |
| | 4338, | 6243, 6275, | 6531, 6563, | 6819, 6851, | 7043, | 7395, 7427, |
| | 4592, | 6307, 6339, | 6595, 6627, | 6883, 6915, | 7075, | 7459, 7491, |
| | 4846, | 6371, 15216, | 6659, 15600, | 6947, 15984, | 7107, | 7523, 16368, |
| | 5100, | 15248, 15280, | 15632, 15664, | 16016, 16048, | 7139, | 16400, 16432, |
| | 5354, | 15312, 15344, | 15696, 15728, | 16080, 16112, | 7171, | 16464, 16496, |
| | 5608, | 15376, 15408, | 15760, 15792, | 16144, 16176, | 7203, | 16528, 16560, |
| | 5862 | 15440, 15472, | 15824, 15856, | 16208, 16240, | 7235 | 16592, 16624, |
| | | 15504, 15536, | 15888, 15920, | 16272, 16304, | | 16656, 16688, |
| | | 15568 | 15952 | 16336 | | 16720 |
| 3595 | 3849, | 6116, 6148, | 6404, 6436, | 6692, 6724, | 6980, | 7268, 7300, |
| | 4103, | 6180, 6212, | 6468, 6500, | 6756, 6788, | 7012, | 7332, 7364, |
| | 4357, | 6244, 6276, | 6532, 6564, | 6820, 6852, | 7044, | 7396, 7428, |
| | 4611, | 6308, 6340, | 6596, 6628, | 6884, 6916, | 7076, | 7460, 7492, |
| | 4865, | 6372, 15217, | 6660, 15601, | 6948, 15985, | 7108, | 7524, 16369, |
| | 5119, | 15249, 15281, | 15633, 15665, | 16017, 16049, | 7140, | 16401, 16433, |
| | 5373, | 15313, 15345, | 15697, 15729, | 16081, 16113, | 7172, | 16465, 16497, |
| | 5627, | 15377, 15409, | 15761, 15793, | 16145, 16177, | 7204, | 16529, 16561, |
| | 5881 | 15441, 15473, | 15825, 15857, | 16209, 16241, | 7236 | 16593, 16625, |
| | | 15505, 15537, | 15889, 15921, | 16273, 16305, | | 16657, 16689, |
| | | 15569 | 15953 | 16337 | | 16721 |
| 3616 | 3870, | 6117, 6149, | 6405, 6437, | 6693, 6725, | 6981, | 7269, 7301, |
| | 4124, | 6181, 6213, | 6469, 6501, | 6757, 6789, | 7013, | 7333, 7365, |
| | 4378, | 6245, 6277, | 6533, 6565, | 6821, 6853, | 7045, | 7397, 7429, |
| | 4632, | 6309, 6341, | 6597, 6629, | 6885, 6917, | 7077, | 7461, 7493, |
| | 4886, | 6373, 15218, | 6661, 15602, | 6949, 15986, | 7109, | 7525, 16370, |
| | 5140, | 15250, 15282, | 15634, 15666, | 16018, 16050, | 7141, | 16402, 16434, |
| | 5394, | 15314, 15346, | 15698, 15730, | 16082, 16114, | 7173, | 16466, 16498, |
| | 5648, | 15378, 15410, | 15762, 15794, | 16146, 16178, | 7205, | 16530, 16562, |
| | 5902 | 15442, 15474, | 15826, 15858, | 16210, 16242, | 7237 | 16594, 16626, |
| | | 15506, 15538, | 15890, 15922, | 16274, 16306, | | 16658, 16690, |
| | | 15570 | 15954 | 16338 | | 16722 |
| 3626 | 3880, | 6118, 6150, | 6406, 6438, | 6694, 6726, | 6982, | 7270, 7302, |
| | 4134, | 6182, 6214, | 6470, 6502, | 6758, 6790, | 7014, | 7334, 7366, |
| | 4388, | 6246, 6278, | 6534, 6566, | 6822, 6854, | 7046, | 7398, 7430, |
| | 4642, | 6310, 6342, | 6598, 6630, | 6886, 6918, | 7078, | 7462, 7494, |
| | 4896, | 6374, 15219, | 6662, 15603, | 6950, 15987, | 7110, | 7526, 16371, |
| | 5150, | 15251, 15283, | 15635, 15667, | 16019, 16051, | 7142, | 16403, 16435, |
| | 5404, | 15315, 15347, | 15699, 15731, | 16083, 16115, | 7174, | 16467, 16499, |
| | 5658, | 15379, 15411, | 15763, 15795, | 16147, 16179, | 7206, | 16531, 16563, |
| | 5912 | 15443, 15475, | 15827, 15859, | 16211, 16243, | 7238 | 16595, 16627, |
| | | 15507, 15539, | 15891, 15923, | 16275, 16307, | | 16659, 16691, |
| | | 15571 | 15955 | 16339 | | 16723 |
| 3627 | 3881, | 6119, 6151, | 6407, 6439, | 6695, 6727, | 6983, | 7271, 7303, |
| | 4135, | 6183, 6215, | 6471, 6503, | 6759, 6791, | 7015, | 7335, 7367, |
| | 4389, | 6247, 6279, | 6535, 6567, | 6823, 6855, | 7047, | 7399, 7431, |
| | 4643, | 6311, 6343, | 6599, 6631, | 6887, 6919, | 7079, | 7463, 7495, |
| | 4897, | 6375, 15220, | 6663, 15604, | 6951, 15988, | 7111, | 7527, 16372, |
| | 5151, | 15252, 15284, | 15636, 15668, | 16020, 16052, | 7143, | 16404, 16436, |
| | 5405, | 15316, 15348, | 15700, 15732, | 16084, 16116, | 7175, | 16468, 16500, |
| | 5659, | 15380, 15412, | 15764, 15796, | 16148, 16180, | 7207, | 16532, 16564, |
| | 5913 | 15444, 15476, | 15828, 15860, | 16212, 16244, | 7239 | 16596, 16628, |
| | | 15508, 15540, | 15892, 15924, | 16276, 16308, | | 16660, 16692, |
| | | 15572 | 15956 | 16340 | | 16724 |
| 3671 | 3925, | 6120, 6152, | 6408, 6440, | 6696, 6728, | 6984, | 7272, 7304, |
| | 4179, | 6184, 6216, | 6472, 6504, | 6760, 6792, | 7016, | 7336, 7368, |
| | 4433, | 6248, 6280, | 6536, 6568, | 6824, 6856, | 7048, | 7400, 7432, |
| | 4687, | 6312, 6344, | 6600, 6632, | 6888, 6920, | 7080, | 7464, 7496, |
| | 4941, | 6376, 15221, | 6664, 15605, | 6952, 15989, | 7112, | 7528, 16373, |
| | 5195, | 15253, 15285, | 15637, 15669, | 16021, 16053, | 7144, | 16405, 16437, |
| | 5449, | 15317, 15349, | 15701, 15733, | 16085, 16117, | 7176, | 16469, 16501, |
| | 5703, | 15381, 15413, | 15765, 15797, | 16149, 16181, | 7208, | 16533, 16565, |
| | 5957 | 15445, 15477, | 15829, 15861, | 16213, 16245, | 7240 | 16597, 16629, |
| | | 15509, 15541, | 15893, 15925, | 16277, 16309, | | 16661, 16693, |
| | | 15573 | 15957 | 16341 | | 16725 |
| 3678 | 3932, | 6121, 6153, | 6409, 6441, | 6697, 6729, | 6985, | 7273, 7305, |
| | 4186, | 6185, 6217, | 6473, 6505, | 6761, 6793, | 7017, | 7337, 7369, |
| | 4440, | 6249, 6281, | 6537, 6569, | 6825, 6857, | 7049, | 7401, 7433, |
| | 4694, | 6313, 6345, | 6601, 6633, | 6889, 6921, | 7081, | 7465, 7497, |
| | 4948, | 6377, 15222, | 6665, 15606, | 6953, 15990, | 7113, | 7529, 16374, |
| | 5202, | 15254, 15286, | 15638, 15670, | 16022, 16054, | 7145, | 16406, 16438, |
| | 5456, | 15318, 15350, | 15702, 15734, | 16086, 16118, | 7177, | 16470, 16502, |
| | 5710, | 15382, 15414, | 15766, 15798, | 16150, 16182, | 7209, | 16534, 16566, |
| | 5964 | 15446, 15478, | 15830, 15862, | 16214, 16246, | 7241 | 16598, 16630, |
| | | 15510, 15542, | 15894, 15926, | 16278, 16310, | | 16662, 16694, |
| | | 15574 | 15958 | 16342 | | 16726 |
| 3680 | 3934, | 6122, 6154, | 6410, 6442, | 6698, 6730, | 6986, | 7274, 7306, |
| | 4188, | 6186, 6218, | 6474, 6506, | 6762, 6794, | 7018, | 7338, 7370, |
| | 4442, | 6250, 6282, | 6538, 6570, | 6826, 6858, | 7050, | 7402, 7434, |
| | 4696, | 6314, 6346, | 6602, 6634, | 6890, 6922, | 7082, | 7466, 7498, |
| | 4950, | 6378, 15223, | 6666, 15607, | 6954, 15991, | 7114, | 7530, 16375, |
| | 5204, | 15255, 15287, | 15639, 15671, | 16023, 16055, | 7146, | 16407, 16439, |
| | 5458, | 15319, 15351, | 15703, 15735, | 16087, 16119, | 7178, | 16471, 16503, |
| | 5712, | 15383, 15415, | 15767, 15799, | 16151, 16183, | 7210, | 16535, 16567, |
| | 5966 | 15447, 15479, | 15831, 15863, | 16215, 16247, | 7242 | 16599, 16631, |
| | | 15511, 15543, | 15895, 15927, | 16279, 16311, | | 16663, 16695, |
| | | 15575 | 15959 | 16343 | | 16727 |
| 3689 | 3943, | 6123, 6155, | 6411, 6443, | 6699, 6731, | 6987, | 7275, 7307, |
| | 4197, | 6187, 6219, | 6475, 6507, | 6763, 6795, | 7019, | 7339, 7371, |
| | 4451, | 6251, 6283, | 6539, 6571, | 6827, 6859, | 7051, | 7403, 7435, |
| | 4705, | 6315, 6347, | 6603, 6635, | 6891, 6923, | 7083, | 7467, 7499, |
| | 4959, | 6379, 15224, | 6667, 15608, | 6955, 15992, | 7115, | 7531, 16376, |
| | 5213, | 15256, 15288, | 15640, 15672, | 16024, 16056, | 7147, | 16408, 16440, |
| | 5467, | 15320, 15352, | 15704, 15736, | 16088, 16120, | 7179, | 16472, 16504, |
| | 5721, | 15384, 15416, | 15768, 15800, | 16152, 16184, | 7211, | 16536, 16568, |
| | 5975 | 15448, 15480, | 15832, 15864, | 16216, 16248, | 7243 | 16600, 16632, |
| | | 15512, 15544, | 15896, 15928, | 16280, 16312, | | 16664, 16696, |
| | | 15576 | 15960 | 16344 | | 16728 |
| 3693 | 3947, | 6124, 6156, | 6412, 6444, | 6700, 6732, | 6988, | 7276, 7308, |
| | 4201, | 6188, 6220, | 6476, 6508, | 6764, 6796, | 7020, | 7340, 7372, |
| | 4455, | 6252, 6284, | 6540, 6572, | 6828, 6860, | 7052, | 7404, 7436, |
| | 4709, | 6316, 6348, | 6604, 6636, | 6892, 6924, | 7084, | 7468, 7500, |
| | 4963, | 6380, 15225, | 6668, 15609, | 6956, 15993, | 7116, | 7532, 16377, |
| | 5217, | 15257, 15289, | 15641, 15673, | 16025, 16057, | 7148, | 16409, 16441, |
| | 5471, | 15321, 15353, | 15705, 15737, | 16089, 16121, | 7180, | 16473, 16505, |
| | 5725, | 15385, 15417, | 15769, 15801, | 16153, 16185, | 7212, | 16537, 16569, |
| | 5979 | 15449, 15481, | 15833, 15865, | 16217, 16249, | 7244 | 16601, 16633, |
| | | 15513, 15545, | 15897, 15929, | 16281, 16313, | | 16665, 16697, |
| | | 15577 | 15961 | 16345 | | 16729 |
| 3697 | 3951, | 6125, 6157, | 6413, 6445, | 6701, 6733, | 6989, | 7277, 7309, |
| | 4205, | 6189, 6221, | 6477, 6509, | 6765, 6797, | 7021, | 7341, 7373, |
| | 4459, | 6253, 6285, | 6541, 6573, | 6829, 6861, | 7053, | 7405, 7437, |
| | 4713, | 6317, 6349, | 6605, 6637, | 6893, 6925, | 7085, | 7469, 7501, |
| | 4967, | 6381, 15226, | 6669, 15610, | 6957, 15994, | 7117, | 7533, 16378, |
| | 5221, | 15258, 15290, | 15642, 15674, | 16026, 16058, | 7149, | 16410, 16442, |
| | 5475, | 15322, 15354, | 15706, 15738, | 16090, 16122, | 7181, | 16474, 16506, |
| | 5729, | 15386, 15418, | 15770, 15802, | 16154, 16186, | 7213, | 16538, 16570, |
| | 5983 | 15450, 15482, | 15834, 15866, | 16218, 16250, | 7245 | 16602, 16634, |
| | | 15514, 15546, | 15898, 15930, | 16282, 16314, | | 16666, 16698, |
| | | 15578 | 15962 | 16346 | | 16730 |
| 3703 | 3957, | 6126, 6158, | 6414, 6446, | 6702, 6734, | 6990, | 7278, 7310, |
| | 4211, | 6190, 6222, | 6478, 6510, | 6766, 6798, | 7022, | 7342, 7374, |
| | 4465, | 6254, 6286, | 6542, 6574, | 6830, 6862, | 7054, | 7406, 7438, |
| | 4719, | 6318, 6350, | 6606, 6638, | 6894, 6926, | 7086, | 7470, 7502, |
| | 4973, | 6382, 15227, | 6670, 15611, | 6958, 15995, | 7118, | 7534, 16379, |
| | 5227, | 15259, 15291, | 15643, 15675, | 16027, 16059, | 7150, | 16411, 16443, |
| | 5481, | 15323, 15355, | 15707, 15739, | 16091, 16123, | 7182, | 16475, 16507, |
| | 5735, | 15387, 15419, | 15771, 15803, | 16155, 16187, | 7214, | 16539, 16571, |
| | 5989 | 15451, 15483, | 15835, 15867, | 16219, 16251, | 7246 | 16603, 16635, |
| | | 15515, 15547, | 15899, 15931, | 16283, 16315, | | 16667, 16699, |
| | | 15579 | 15963 | 16347 | | 16731 |
| 3704 | 3958, | 6127, 6159, | 6415, 6447, | 6703, 6735, | 6991, | 7279, 7311, |
| | 4212, | 6191, 6223, | 6479, 6511, | 6767, 6799, | 7023, | 7343, 7375, |
| | 4466, | 6255, 6287, | 6543, 6575, | 6831, 6863, | 7055, | 7407, 7439, |
| | 4720, | 6319, 6351, | 6607, 6639, | 6895, 6927, | 7087, | 7471, 7503, |
| | 4974, | 6383, 15228, | 6671, 15612, | 6959, 15996, | 7119, | 7535, 16380, |
| | 5228, | 15260, 15292, | 15644, 15676, | 16028, 16060, | 7151, | 16412, 16444, |
| | 5482, | 15324, 15356, | 15708, 15740, | 16092, 16124, | 7183, | 16476, 16508, |
| | 5736, | 15388, 15420, | 15772, 15804, | 16156, 16188, | 7215, | 16540, 16572, |
| | 5990 | 15452, 15484, | 15836, 15868, | 16220, 16252, | 7247 | 16604, 16636, |
| | | 15516, 15548, | 15900, 15932, | 16284, 16316, | | 16668, 16700, |
| | | 15580 | 15964 | 16348 | | 16732 |
| 3728 | 3982, | 6128, 6160, | 6416, 6448, | 6704, 6736, | 6992, | 7280, 7312, |
| | 4236, | 6192, 6224, | 6480, 6512, | 6768, 6800, | 7024, | 7344, 7376, |
| | 4490, | 6256, 6288, | 6544, 6576, | 6832, 6864, | 7056, | 7408, 7440, |
| | 4744, | 6320, 6352, | 6608, 6640, | 6896, 6928, | 7088, | 7472, 7504, |
| | 4998, | 6384, 15229, | 6672, 15613, | 6960, 15997, | 7120, | 7536, 16381, |
| | 5252, | 15261, 15293, | 15645, 15677, | 16029, 16061, | 7152, | 16413, 16445, |
| | 5506, | 15325, 15357, | 15709, 15741, | 16093, 16125, | 7184, | 16477, 16509, |
| | 5760, | 15389, 15421, | 15773, 15805, | 16157, 16189, | 7216, | 16541, 16573, |
| | 6014 | 15453, 15485, | 15837, 15869, | 16221, 16253, | 7248 | 16605, 16637, |
| | | 15517, 15549, | 15901, 15933, | 16285, 16317, | | 16669, 16701, |
| | | 15581 | 15965 | 16349 | | 16733 |
| 3733 | 3987, | 6129, 6161, | 6417, 6449, | 6705, 6737, | 6993, | 7281, 7313, |
| | 4241, | 6193, 6225, | 6481, 6513, | 6769, 6801, | 7025, | 7345, 7377, |
| | 4495, | 6257, 6289, | 6545, 6577, | 6833, 6865, | 7057, | 7409, 7441, |
| | 4749, | 6321, 6353, | 6609, 6641, | 6897, 6929, | 7089, | 7473, 7505, |
| | 5003, | 6385, 15230, | 6673, 15614, | 6961, 15998, | 7121, | 7537, 16382, |
| | 5257, | 15262, 15294, | 15646, 15678, | 16030, 16062, | 7153, | 16414, 16446, |
| | 5511, | 15326, 15358, | 15710, 15742, | 16094, 16126, | 7185, | 16478, 16510, |
| | 5765, | 15390, 15422, | 15774, 15806, | 16158, 16190, | 7217, | 16542, 16574, |
| | 6019 | 15454, 15486, | 15838, 15870, | 16222, 16254, | 7249 | 16606, 16638, |
| | | 15518, 15550, | 15902, 15934, | 16286, 16318, | | 16670, 16702, |
| | | 15582 | 15966 | 16350 | | 16734 |
| 3736 | 3990, | 6130, 6162, | 6418, 6450, | 6706, 6738, | 6994, | 7282, 7314, |
| | 4244, | 6194, 6226, | 6482, 6514, | 6770, 6802, | 7026, | 7346, 7378, |
| | 4498, | 6258, 6290, | 6546, 6578, | 6834, 6866, | 7058, | 7410, 7442, |
| | 4752, | 6322, 6354, | 6610, 6642, | 6898, 6930, | 7090, | 7474, 7506, |
| | 5006, | 6386, 15231, | 6674, 15615, | 6962, 15999, | 7122, | 7538, 16383, |
| | 5260, | 15263, 15295, | 15647, 15679, | 16031, 16063, | 7154, | 16415, 16447, |
| | 5514, | 15327, 15359, | 15711, 15743, | 16095, 16127, | 7186, | 16479, 16511, |
| | 5768, | 15391, 15423, | 15775, 15807, | 16159, 16191, | 7218, | 16543, 16575, |
| | 6022 | 15455, 15487, | 15839, 15871, | 16223, 16255, | 7250 | 16607, 16639, |
| | | 15519, 15551, | 15903, 15935, | 16287, 16319, | | 16671, 16703, |
| | | 15583 | 15967 | 16351 | | 16735 |
| 3775 | 4029, | 6131, 6163, | 6419, 6451, | 6707, 6739, | 6995, | 7283, 7315, |
| | 4283, | 6195, 6227, | 6483, 6515, | 6771, 6803, | 7027, | 7347, 7379, |
| | 4537, | 6259, 6291, | 6547, 6579, | 6835, 6867, | 7059, | 7411, 7443, |
| | 4791, | 6323, 6355, | 6611, 6643, | 6899, 6931, | 7091, | 7475, 7507, |
| | 5045, | 6387, 15232, | 6675, 15616, | 6963, 16000, | 7123, | 7539, 16384, |
| | 5299, | 15264, 15296, | 15648, 15680, | 16032, 16064, | 7155, | 16416, 16448, |
| | 5553, | 15328, 15360, | 15712, 15744, | 16096, 16128, | 7187, | 16480, 16512, |
| | 5807, | 15392, 15424, | 15776, 15808, | 16160, 16192, | 7219, | 16544, 16576, |
| | 6061 | 15456, 15488, | 15840, 15872, | 16224, 16256, | 7251 | 16608, 16640, |
| | | 15520, 15552, | 15904, 15936, | 16288, 16320, | | 16672, 16704, |
| | | 15584 | 15968 | 16352 | | 16736 |
| 3785 | 4039, | 6132, 6164, | 6420, 6452, | 6708, 6740, | 6996, | 7284, 7316, |
| | 4293, | 6196, 6228, | 6484, 6516, | 6772, 6804, | 7028, | 7348, 7380, |
| | 4547, | 6260, 6292, | 6548, 6580, | 6836, 6868, | 7060, | 7412, 7444, |
| | 4801, | 6324, 6356, | 6612, 6644, | 6900, 6932, | 7092, | 7476, 7508, |
| | 5055, | 6388, 15233, | 6676, 15617, | 6964, 16001, | 7124, | 7540, 16385, |
| | 5309, | 15265, 15297, | 15649, 15681, | 16033, 16065, | 7156, | 16417, 16449, |
| | 5563, | 15329, 15361, | 15713, 15745, | 16097, 16129, | 7188, | 16481, 16513, |
| | 5817, | 15393, 15425, | 15777, 15809, | 16161, 16193, | 7220, | 16545, 16577, |
| | 6071 | 15457, 15489, | 15841, 15873, | 16225, 16257, | 7252 | 16609, 16641, |
| | | 15521, 15553, | 15905, 15937, | 16289, 16321, | | 16673, 16705, |
| | | 15585 | 15969 | 16353 | | 16737 |
| 3790 | 4044, | 6133, 6165, | 6421, 6453, | 6709, 6741, | 6997, | 7285, 7317, |
| | 4298, | 6197, 6229, | 6485, 6517, | 6773, 6805, | 7029, | 7349, 7381, |
| | 4552, | 6261, 6293, | 6549, 6581, | 6837, 6869, | 7061, | 7413, 7445, |
| | 4806, | 6325, 6357, | 6613, 6645, | 6901, 6933, | 7093, | 7477, 7509, |
| | 5060, | 6389, 15234, | 6677, 15618, | 6965, 16002, | 7125, | 7541, 16386, |
| | 5314, | 15266, 15298, | 15650, 15682, | 16034, 16066, | 7157, | 16418, 16450, |
| | 5568, | 15330, 15362, | 15714, 15746, | 16098, 16130, | 7189, | 16482, 16514, |
| | 5822, | 15394, 15426, | 15778, 15810, | 16162, 16194, | 7221, | 16546, 16578, |
| | 6076 | 15458, 15490, | 15842, 15874, | 16226, 16258, | 7253 | 16610, 16642, |
| | | 15522, 15554, | 15906, 15938, | 16290, 16322, | | 16674, 16706, |
| | | 15586 | 15970 | 16354 | | 16738 |
| 3795 | 4049, | 6134, 6166, | 6422, 6454, | 6710, 6742, | 6998, | 7286, 7318, |
| | 4303, | 6198, 6230, | 6486, 6518, | 6774, 6806, | 7030, | 7350, 7382, |
| | 4557, | 6262, 6294, | 6550, 6582, | 6838, 6870, | 7062, | 7414, 7446, |
| | 4811, | 6326, 6358, | 6614, 6646, | 6902, 6934, | 7094, | 7478, 7510, |
| | 5065, | 6390, 15235, | 6678, 15619, | 6966, 16003, | 7126, | 7542, 16387, |
| | 5319, | 15267, 15299, | 15651, 15683, | 16035, 16067, | 7158, | 16419, 16451, |
| | 5573, | 15331, 15363, | 15715, 15747, | 16099, 16131, | 7190, | 16483, 16515, |
| | 5827, | 15395, 15427, | 15779, 15811, | 16163, 16195, | 7222, | 16547, 16579, |
| | 6081 | 15459, 15491, | 15843, 15875, | 16227, 16259, | 7254 | 16611, 16643, |
| | | 15523, 15555, | 15907, 15939, | 16291, 16323, | | 16675, 16707, |
| | | 15587 | 15971 | 16355 | | 16739 |
| 3801 | 4055, | 6135, 6167, | 6423, 6455, | 6711, 6743, | 6999, | 7287, 7319, |
| | 4309, | 6199, 6231, | 6487, 6519, | 6775, 6807, | 7031, | 7351, 7383, |
| | 4563, | 6263, 6295, | 6551, 6583, | 6839, 6871, | 7063, | 7415, 7447, |
| | 4817, | 6327, 6359, | 6615, 6647, | 6903, 6935, | 7095, | 7479, 7511, |
| | 5071, | 6391, 15236, | 6679, 15620, | 6967, 16004, | 7127, | 7543, 16388, |
| | 5325, | 15268, 15300, | 15652, 15684, | 16036, 16068, | 7159, | 16420, 16452, |
| | 5579, | 15332, 15364, | 15716, 15748, | 16100, 16132, | 7191, | 16484, 16516, |
| | 5833, | 15396, 15428, | 15780, 15812, | 16164, 16196, | 7223, | 16548, 16580, |
| | 6087 | 15460, 15492, | 15844, 15876, | 16228, 16260, | 7255 | 16612, 16644, |
| | | 15524, 15556, | 15908, 15940, | 16292, 16324, | | 16676, 16708, |
| | | 15588 | 15972 | 16356 | | 16740 |
| 3803 | 4057, | 6136, 6168, | 6424, 6456, | 6712, 6744, | 7000, | 7288, 7320, |
| | 4311, | 6200, 6232, | 6488, 6520, | 6776, 6808, | 7032, | 7352, 7384, |
| | 4565, | 6264, 6296, | 6552, 6584, | 6840, 6872, | 7064, | 7416, 7448, |
| | 4819, | 6328, 6360, | 6616, 6648, | 6904, 6936, | 7096, | 7480, 7512, |
| | 5073, | 6392, 15237, | 6680, 15621, | 6968, 16005, | 7128, | 7544, 16389, |
| | 5327, | 15269, 15301, | 15653, 15685, | 16037, 16069, | 7160, | 16421, 16453, |
| | 5581, | 15333, 15365, | 15717, 15749, | 16101, 16133, | 7192, | 16485, 16517, |
| | 5835, | 15397, 15429, | 15781, 15813, | 16165, 16197, | 7224, | 16549, 16581, |
| | 6089 | 15461, 15493, | 15845, 15877, | 16229, 16261, | 7256 | 16613, 16645, |
| | | 15525, 15557, | 15909, 15941, | 16293, 16325, | | 16677, 16709, |
| | | 15589 | 15973 | 16357 | | 16741 |
| 3805 | 4059, | 6137, 6169, | 6425, 6457, | 6713, 6745, | 7001, | 7289, 7321, |
| | 4313, | 6201, 6233, | 6489, 6521, | 6777, 6809, | 7033, | 7353, 7385, |
| | 4567, | 6265, 6297, | 6553, 6585, | 6841, 6873, | 7065, | 7417, 7449, |
| | 4821, | 6329, 6361, | 6617, 6649, | 6905, 6937, | 7097, | 7481, 7513, |
| | 5075, | 6393, 15238, | 6681, 15622, | 6969, 16006, | 7129, | 7545, 16390, |
| | 5329, | 15270, 15302, | 15654, 15686, | 16038, 16070, | 7161, | 16422, 16454, |
| | 5583, | 15334, 15366, | 15718, 15750, | 16102, 16134, | 7193, | 16486, 16518, |
| | 5837, | 15398, 15430, | 15782, 15814, | 16166, 16198, | 7225, | 16550, 16582, |
| | 6091 | 15462, 15494, | 15846, 15878, | 16230, 16262, | 7257 | 16614, 16646, |
| | | 15526, 15558, | 15910, 15942, | 16294, 16326, | | 16678, 16710, |
| | | 15590 | 15974 | 16358 | | 16742 |
| 3811 | 4065, | 6138, 6170, | 6426, 6458, | 6714, 6746, | 7002, | 7290, 7322, |
| | 4319, | 6202, 6234, | 6490, 6522, | 6778, 6810, | 7034, | 7354, 7386, |
| | 4573, | 6266, 6298, | 6554, 6586, | 6842, 6874, | 7066, | 7418, 7450, |
| | 4827, | 6330, 6362, | 6618, 6650, | 6906, 6938, | 7098, | 7482, 7514, |
| | 5081, | 6394, 15239, | 6682, 15623, | 6970, 16007, | 7130, | 7546, 16391, |
| | 5335, | 15271, 15303, | 15655, 15687, | 16039, 16071, | 7162, | 16423, 16455, |
| | 5589, | 15335, 15367, | 15719, 15751, | 16103, 16135, | 7194, | 16487, 16519, |
| | 5843, | 15399, 15431, | 15783, 15815, | 16167, 16199, | 7226, | 16551, 16583, |
| | 6097 | 15463, 15495, | 15847, 15879, | 16231, 16263, | 7258 | 16615, 16647, |
| | | 15527, 15559, | 15911, 15943, | 16295, 16327, | | 16679, 16711, |
| | | 15591 | 15975 | 16359 | | 16743 |

TABLE 3.2
Preferred LASV prefusion-stabilized (GPCmut2) constructs

| | | RNA designs and SEQ ID NOs | | | | |
|---|---|---|---|---|---|---|
| PRT | CDS | a-1 | a-2 | b-4 | i-2 | i-3 |

| | | | | | |
|---|---|---|---|---|---|
| 22949 | 22981, | 23109, 23141, | 23493, 23525, | 23877, 23909, | 24261, 24293, |
| | 23013, | 23173, 23205, | 23557, 23589, | 23941, 23973, | 24325, 24357, |
| | 23045, | 23237, 23269, | 23621, 23653, | 24005, 24037, | 24389, 24421, |
| | 23077 | 23301, 23333, | 23685, 23717, | 24069, 24101, | 24453, 24485, |
| | | 23365, 23397, | 23749, 23781, | 24133, 24165, | 24517, 24549, |
| | | 23429, 23461 | 23813, 23845 | 24197, 24229 | 24581, 24613 |
| 22950 | 22982, | 23110, 23142, | 23494, 23526, | 23878, 23910, | 24262, 24294, |
| | 23014, | 23174, 23206, | 23558, 23590, | 23942, 23974, | 24326, 24358, |
| | 23046, | 23238, 23270, | 23622, 23654, | 24006, 24038, | 24390, 24422, |
| | 23078 | 23302, 23334, | 23686, 23718, | 24070, 24102, | 24454, 24486, |
| | | 23366, 23398, | 23750, 23782, | 24134, 24166, | 24518, 24550, |
| | | 23430, 23462 | 23814, 23846 | 24198, 24230 | 24582, 24614 |
| 22951 | 22983, | 23111, 23143, | 23495, 23527, | 23879, 23911, | 24263, 24295, |
| | 23015, | 23175, 23207, | 23559, 23591, | 23943, 23975, | 24327, 24359, |
| | 23047, | 23239, 23271, | 23623, 23655, | 24007, 24039, | 24391, 24423, |
| | 23079 | 23303, 23335, | 23687, 23719, | 24071, 24103, | 24455, 24487, |
| | | 23367, 23399, | 23751, 23783, | 24135, 24167, | 24519, 24551, |
| | | 23431, 23463 | 23815, 23847 | 24199, 24231 | 24583, 24615 |
| 22952 | 22984, | 23112, 23144, | 23496, 23528, | 23880, 23912, | 24264, 24296, |
| | 23016, | 23176, 23208, | 23560, 23592, | 23944, 23976, | 24328, 24360, |
| | 23048, | 23240, 23272, | 23624, 23656, | 24008, 24040, | 24392, 24424, |
| | 23080 | 23304, 23336, | 23688, 23720, | 24072, 24104, | 24456, 24488, |
| | | 23368, 23400, | 23752, 23784, | 24136, 24168, | 24520, 24552, |
| | | 23432, 23464 | 23816, 23848 | 24200, 24232 | 24584, 24616 |
| 22953 | 22985, | 23113, 23145, | 23497, 23529, | 23881, 23913, | 24265, 24297, |
| | 23017, | 23177, 23209, | 23561, 23593, | 23945, 23977, | 24329, 24361, |
| | 23049, | 23241, 23273, | 23625, 23657, | 24009, 24041, | 24393, 24425, |
| | 23081 | 23305, 23337, | 23689, 23721, | 24073, 24105, | 24457, 24489, |
| | | 23369, 23401, | 23753, 23785, | 24137, 24169, | 24521, 24553, |
| | | 23433, 23465 | 23817, 23849 | 24201, 24233 | 24585, 24617 |
| 22954 | 22986, | 23114, 23146, | 23498, 23530, | 23882, 23914, | 24266, 24298, |
| | 23018, | 23178, 23210, | 23562, 23594, | 23946, 23978, | 24330, 24362, |
| | 23050, | 23242, 23274, | 23626, 23658, | 24010, 24042, | 24394, 24426, |
| | 23082 | 23306, 23338, | 23690, 23722, | 24074, 24106, | 24458, 24490, |
| | | 23370, 23402, | 23754, 23786, | 24138, 24170, | 24522, 24554, |
| | | 23434, 23466 | 23818, 23850 | 24202, 24234 | 24586, 24618 |
| 22955 | 22987, | 23115, 23147, | 23499, 23531, | 23883, 23915, | 24267, 24299, |
| | 23019, | 23179, 23211, | 23563, 23595, | 23947, 23979, | 24331, 24363, |
| | 23051, | 23243, 23275, | 23627, 23659, | 24011, 24043, | 24395, 24427, |
| | 23083 | 23307, 23339, | 23691, 23723, | 24075, 24107, | 24459, 24491, |
| | | 23371, 23403, | 23755, 23787, | 24139, 24171, | 24523, 24555, |
| | | 23435, 23467 | 23819, 23851 | 24203, 24235 | 24587, 24619 |
| 22956 | 22988, | 23116, 23148, | 23500, 23532, | 23884, 23916, | 24268, 24300, |
| | 23020, | 23180, 23212, | 23564, 23596, | 23948, 23980, | 24332, 24364, |
| | 23052, | 23244, 23276, | 23628, 23660, | 24012, 24044, | 24396, 24428, |
| | 23084 | 23308, 23340, | 23692, 23724, | 24076, 24108, | 24460, 24492, |
| | | 23372, 23404, | 23756, 23788, | 24140, 24172, | 24524, 24556, |
| | | 23436, 23468 | 23820, 23852 | 24204, 24236 | 24588, 24620 |
| 22957 | 22989, | 23117, 23149, | 23501, 23533, | 23885, 23917, | 24269, 24301, |
| | 23021, | 23181, 23213, | 23565, 23597, | 23949, 23981, | 24333, 24365, |
| | 23053, | 23245, 23277, | 23629, 23661, | 24013, 24045, | 24397, 24429, |
| | 23085 | 23309, 23341, | 23693, 23725, | 24077, 24109, | 24461, 24493, |
| | | 23373, 23405, | 23757, 23789, | 24141, 24173, | 24525, 24557, |
| | | 23437, 23469 | 23821, 23853 | 24205, 24237 | 24589, 24621 |
| 22958 | 22990, | 23118, 23150, | 23502, 23534, | 23886, 23918, | 24270, 24302, |
| | 23022, | 23182, 23214, | 23566, 23598, | 23950, 23982, | 24334, 24366, |
| | 23054, | 23246, 23278, | 23630, 23662, | 24014, 24046, | 24398, 24430, |
| | 23086 | 23310, 23342, | 23694, 23726, | 24078, 24110, | 24462, 24494, |
| | | 23374, 23406, | 23758, 23790, | 24142, 24174, | 24526, 24558, |
| | | 23438, 23470 | 23822, 23854 | 24206, 24238 | 24590, 24622 |
| 22959 | 22991, | 23119, 23151, | 23503, 23535, | 23887, 23919, | 24271, 24303, |
| | 23023, | 23183, 23215, | 23567, 23599, | 23951, 23983, | 24335, 24367, |
| | 23055, | 23247, 23279, | 23631, 23663, | 24015, 24047, | 24399, 24431, |
| | 23087 | 23311, 23343, | 23695, 23727, | 24079, 24111, | 24463, 24495, |
| | | 23375, 23407, | 23759, 23791, | 24143, 24175, | 24527, 24559, |
| | | 23439, 23471 | 23823, 23855 | 24207, 24239 | 24591, 24623 |
| 22960 | 22992, | 23120, 23152, | 23504, 23536, | 23888, 23920, | 24272, 24304, |
| | 23024, | 23184, 23216, | 23568, 23600, | 23952, 23984, | 24336, 24368, |
| | 23056, | 23248, 23280, | 23632, 23664, | 24016, 24048, | 24400, 24432, |
| | 23088 | 23312, 23344, | 23696, 23728, | 24080, 24112, | 24464, 24496, |
| | | 23376, 23408, | 23760, 23792, | 24144, 24176, | 24528, 24560, |
| | | 23440, 23472 | 23824, 23856 | 24208, 24240 | 24592, 24624 |
| 22961 | 22993, | 23121, 23153, | 23505, 23537, | 23889, 23921, | 24273, 24305, |
| | 23025, | 23185, 23217, | 23569, 23601, | 23953, 23985, | 24337, 24369, |
| | 23057, | 23249, 23281, | 23633, 23665, | 24017, 24049, | 24401, 24433, |
| | 23089 | 23313, 23345, | 23697, 23729, | 24081, 24113, | 24465, 24497, |
| | | 23377, 23409, | 23761, 23793, | 24145, 24177, | 24529, 24561, |
| | | 23441, 23473 | 23825, 23857 | 24209, 24241 | 24593, 24625 |
| 22962 | 22994, | 23122, 23154, | 23506, 23538, | 23890, 23922, | 24274, 24306, |
| | 23026, | 23186, 23218, | 23570, 23602, | 23954, 23986, | 24338, 24370, |
| | 23058, | 23250, 23282, | 23634, 23666, | 24018, 24050, | 24402, 24434, |
| | 23090 | 23314, 23346, | 23698, 23730, | 24082, 24114, | 24466, 24498, |
| | | 23378, 23410, | 23762, 23794, | 24146, 24178, | 24530, 24562, |
| | | 23442, 23474 | 23826, 23858 | 24210, 24242 | 24594, 24626 |
| 22963 | 22995, | 23123, 23155, | 23507, 23539, | 23891, 23923, | 24275, 24307, |
| | 23027, | 23187, 23219, | 23571, 23603, | 23955, 23987, | 24339, 24371, |
| | 23059, | 23251, 23283, | 23635, 23667, | 24019, 24051, | 24403, 24435, |
| | 23091 | 23315, 23347, | 23699, 23731, | 24083, 24115, | 24467, 24499, |
| | | 23379, 23411, | 23763, 23795, | 24147, 24179, | 24531, 24563, |
| | | 23443, 23475 | 23827, 23859 | 24211, 24243 | 24595, 24627 |
| 22964 | 22996, | 23124, 23156, | 23508, 23540, | 23892, 23924, | 24276, 24308, |
| | 23028, | 23188, 23220, | 23572, 23604, | 23956, 23988, | 24340, 24372, |
| | 23060, | 23252, 23284, | 23636, 23668, | 24020, 24052, | 24404, 24436, |
| | 23092 | 23316, 23348, | 23700, 23732, | 24084, 24116, | 24468, 24500, |
| | | 23380, 23412, | 23764, 23796, | 24148, 24180, | 24532, 24564, |
| | | 23444, 23476 | 23828, 23860 | 24212, 24244 | 24596, 24628 |
| 22965 | 22997, | 23125, 23157, | 23509, 23541, | 23893, 23925, | 24277, 24309, |
| | 23029, | 23189, 23221, | 23573, 23605, | 23957, 23989, | 24341, 24373, |
| | 23061, | 23253, 23285, | 23637, 23669, | 24021, 24053, | 24405, 24437, |
| | 23093 | 23317, 23349, | 23701, 23733, | 24085, 24117, | 24469, 24501, |
| | | 23381, 23413, | 23765, 23797, | 24149, 24181, | 24533, 24565, |
| | | 23445, 23477 | 23829, 23861 | 24213, 24245 | 24597, 24629 |
| 22966 | 22998, | 23126, 23158, | 23510, 23542, | 23894, 23926, | 24278, 24310, |
| | 23030, | 23190, 23222, | 23574, 23606, | 23958, 23990, | 24342, 24374, |
| | 23062, | 23254, 23286, | 23638, 23670, | 24022, 24054, | 24406, 24438, |
| | 23094 | 23318, 23350, | 23702, 23734, | 24086, 24118, | 24470, 24502, |
| | | 23382, 23414, | 23766, 23798, | 24150, 24182, | 24534, 24566, |
| | | 23446, 23478 | 23830, 23862 | 24214, 24246 | 24598, 24630 |
| 22967 | 22999, | 23127, 23159, | 23511, 23543, | 23895, 23927, | 24279, 24311, |
| | 23031, | 23191, 23223, | 23575, 23607, | 23959, 23991, | 24343, 24375, |
| | 23063, | 23255, 23287, | 23639, 23671, | 24023, 24055, | 24407, 24439, |
| | 23095 | 23319, 23351, | 23703, 23735, | 24087, 24119, | 24471, 24503, |
| | | 23383, 23415, | 23767, 23799, | 24151, 24183, | 24535, 24567, |
| | | 23447, 23479 | 23831, 23863 | 24215, 24247 | 24599, 24631 |
| 22968 | 23000, | 23128, 23160, | 23512, 23544, | 23896, 23928, | 24280, 24312, |
| | 23032, | 23192, 23224, | 23576, 23608, | 23960, 23992, | 24344, 24376, |
| | 23064, | 23256, 23288, | 23640, 23672, | 24024, 24056, | 24408, 24440, |
| | 23096 | 23320, 23352, | 23704, 23736, | 24088, 24120, | 24472, 24504, |
| | | 23384, 23416, | 23768, 23800, | 24152, 24184, | 24536, 24568, |
| | | 23448, 23480 | 23832, 23864 | 24216, 24248 | 24600, 24632 |
| 22969 | 23001, | 23129, 23161, | 23513, 23545, | 23897, 23929, | 24281, 24313, |
| | 23033, | 23193, 23225, | 23577, 23609, | 23961, 23993, | 24345, 24377, |
| | 23065, | 23257, 23289, | 23641, 23673, | 24025, 24057, | 24409, 24441, |
| | 23097 | 23321, 23353, | 23705, 23737, | 24089, 24121, | 24473, 24505, |
| | | 23385, 23417, | 23769, 23801, | 24153, 24185, | 24537, 24569, |
| | | 23449, 23481 | 23833, 23865 | 24217, 24249 | 24601, 24633 |
| 22970 | 23002, | 23130, 23162, | 23514, 23546, | 23898, 23930, | 24282, 24314, |
| | 23034, | 23194, 23226, | 23578, 23610, | 23962, 23994, | 24346, 24378, |
| | 23066, | 23258, 23290, | 23642, 23674, | 24026, 24058, | 24410, 24442, |
| | 23098 | 23322, 23354, | 23706, 23738, | 24090, 24122, | 24474, 24506, |
| | | 23386, 23418, | 23770, 23802, | 24154, 24186, | 24538, 24570, |
| | | 23450, 23482 | 23834, 23866 | 24218, 24250 | 24602, 24634 |
| 22971 | 23003, | 23131, 23163, | 23515, 23547, | 23899, 23931, | 24283, 24315, |
| | 23035, | 23195, 23227, | 23579, 23611, | 23963, 23995, | 24347, 24379, |
| | 23067, | 23259, 23291, | 23643, 23675, | 24027, 24059, | 24411, 24443, |
| | 23099 | 23323, 23355, | 23707, 23739, | 24091, 24123, | 24475, 24507, |
| | | 23387, 23419, | 23771, 23803, | 24155, 24187, | 24539, 24571, |
| | | 23451, 23483 | 23835, 23867 | 24219, 24251 | 24603, 24635 |
| 22972 | 23004, | 23132, 23164, | 23516, 23548, | 23900, 23932, | 24284, 24316, |
| | 23036, | 23196, 23228, | 23580, 23612, | 23964, 23996, | 24348, 24380, |
| | 23068, | 23260, 23292, | 23644, 23676, | 24028, 24060, | 24412, 24444, |
| | 23100 | 23324, 23356, | 23708, 23740, | 24092, 24124, | 24476, 24508, |
| | | 23388, 23420, | 23772, 23804, | 24156, 24188, | 24540, 24572, |
| | | 23452, 23484 | 23836, 23868 | 24220, 24252 | 24604, 24636 |
| 22973 | 23005, | 23133, 23165, | 23517, 23549, | 23901, 23933, | 24285, 24317, |
| | 23037, | 23197, 23229, | 23581, 23613, | 23965, 23997, | 24349, 24381, |
| | 23069, | 23261, 23293, | 23645, 23677, | 24029, 24061, | 24413, 24445, |
| | 23101 | 23325, 23357, | 23709, 23741, | 24093, 24125, | 24477, 24509, |
| | | 23389, 23421, | 23773, 23805, | 24157, 24189, | 24541, 24573, |
| | | 23453, 23485 | 23837, 23869 | 24221, 24253 | 24605, 24637 |
| 22974 | 23006, | 23134, 23166, | 23518, 23550, | 23902, 23934, | 24286, 24318, |
| | 23038, | 23198, 23230, | 23582, 23614, | 23966, 23998, | 24350, 24382, |
| | 23070, | 23262, 23294, | 23646, 23678, | 24030, 24062, | 24414, 24446, |
| | 23102 | 23326, 23358, | 23710, 23742, | 24094, 24126, | 24478, 24510, |
| | | 23390, 23422, | 23774, 23806, | 24158, 24190, | 24542, 24574, |
| | | 23454, 23486 | 23838, 23870 | 24222, 24254 | 24606, 24638 |
| 22975 | 23007, | 23135, 23167, | 23519, 23551, | 23903, 23935, | 24287, 24319, |
| | 23039, | 23199, 23231, | 23583, 23615, | 23967, 23999, | 24351, 24383, |
| | 23071, | 23263, 23295, | 23647, 23679, | 24031, 24063, | 24415, 24447, |
| | 23103 | 23327, 23359, | 23711, 23743, | 24095, 24127, | 24479, 24511, |
| | | 23391, 23423, | 23775, 23807, | 24159, 24191, | 24543, 24575, |
| | | 23455, 23487 | 23839, 23871 | 24223, 24255 | 24607, 24639 |
| 22976 | 23008, | 23136, 23168, | 23520, 23552, | 23904, 23936, | 24288, 24320, |
| | 23040, | 23200, 23232, | 23584, 23616, | 23968, 24000, | 24352, 24384, |
| | 23072, | 23264, 23296, | 23648, 23680, | 24032, 24064, | 24416, 24448, |
| | 23104 | 23328, 23360, | 23712, 23744, | 24096, 24128, | 24480, 24512, |
| | | 23392, 23424, | 23776, 23808, | 24160, 24192, | 24544, 24576, |
| | | 23456, 23488 | 23840, 23872 | 24224, 24256 | 24608, 24640 |
| 22977 | 23009, | 23137, 23169, | 23521, 23553, | 23905, 23937, | 24289, 24321, |
| | 23041, | 23201, 23233, | 23585, 23617, | 23969, 24001, | 24353, 24385, |
| | 23073, | 23265, 23297, | 23649, 23681, | 24033, 24065, | 24417, 24449, |
| | 23105 | 23329, 23361, | 23713, 23745, | 24097, 24129, | 24481, 24513, |
| | | 23393, 23425, | 23777, 23809, | 24161, 24193, | 24545, 24577, |
| | | 23457, 23489 | 23841, 23873 | 24225, 24257 | 24609, 24641 |
| 22978 | 23010, | 23138, 23170, | 23522, 23554, | 23906, 23938, | 24290, 24322, |
| | 23042, | 23202, 23234, | 23586, 23618, | 23970, 24002, | 24354, 24386, |
| | 23074, | 23266, 23298, | 23650, 23682, | 24034, 24066, | 24418, 24450, |
| | 23106 | 23330, 23362, | 23714, 23746, | 24098, 24130, | 24482, 24514, |
| | | 23394, 23426, | 23778, 23810, | 24162, 24194, | 24546, 24578, |
| | | 23458, 23490 | 23842, 23874 | 24226, 24258 | 24610, 24642 |
| 22979 | 23011, | 23139, 23171, | 23523, 23555, | 23907, 23939, | 24291, 24323, |
| | 23043, | 23203, 23235, | 23587, 23619, | 23971, 24003, | 24355, 24387, |
| | 23075, | 23267, 23299, | 23651, 23683, | 24035, 24067, | 24419, 24451, |
| | 23107 | 23331, 23363, | 23715, 23747, | 24099, 24131, | 24483, 24515, |
| | | 23395, 23427, | 23779, 23811, | 24163, 24195, | 24547, 24579, |
| | | 23459, 23491 | 23843, 23875 | 24227, 24259 | 24611, 24643 |
| 22980 | 23012, | 23140, 23172, | 23524, 23556, | 23908, 23940, | 24292, 24324, |
| | 23044, | 23204, 23236, | 23588, 23620, | 23972, 24004, | 24356, 24388, |
| | 23076, | 23268, 23300, | 23652, 23684, | 24036, 24068, | 24420, 24452, |
| | 23108 | 23332, 23364, | 23716, 23748, | 24100, 24132, | 24484, 24516, |
| | | 23396, 23428, | 23780, 23812, | 24164, 24196, | 24548, 24580, |
| | | 23460, 23492 | 23844, 23876 | 24228, 24260 | 24612, 24644 |

TABLE 3.3
Preferred LASV prefusion-stabilized (GPCmut3) constructs

| | | RNA designs and SEQ ID NOs | | | | |
|---|---|---|---|---|---|---|
| PRT | CDS | a-1 | a-2 | b-4 | i-2 | i-3 |

| | | | | | |
|---|---|---|---|---|---|
| 24645 | 24677, | 24805, 24837, | 25189, 25221, | 25573, 25605, | 25957, 25989, |
| | 24709, | 24869, 24901, | 25253, 25285, | 25637, 25669, | 26021, 26053, |
| | 24741, | 24933, 24965, | 25317, 25349, | 25701, 25733, | 26085, 26117, |
| | 24773 | 24997, 25029, | 25381, 25413, | 25765, 25797, | 26149, 26181, |
| | | 25061, 25093, | 25445, 25477, | 25829, 25861, | 26213, 26245, |
| | | 25125, 25157 | 25509, 25541 | 25893, 25925 | 26277, 26309 |
| 24646 | 24678, | 24806, 24838, | 25190, 25222, | 25574, 25606, | 25958, 25990, |
| | 24710, | 24870, 24902, | 25254, 25286, | 25638, 25670, | 26022, 26054, |
| | 24742, | 24934, 24966, | 25318, 25350, | 25702, 25734, | 26086, 26118, |
| | 24774 | 24998, 25030, | 25382, 25414, | 25766, 25798, | 26150, 26182, |
| | | 25062, 25094, | 25446, 25478, | 25830, 25862, | 26214, 26246, |
| | | 25126, 25158 | 25510, 25542 | 25894, 25926 | 26278, 26310 |
| 24647 | 24679, | 24807, 24839, | 25191, 25223, | 25575, 25607, | 25959, 25991, |
| | 24711, | 24871, 24903, | 25255, 25287, | 25639, 25671, | 26023, 26055, |
| | 24743, | 24935, 24967, | 25319, 25351, | 25703, 25735, | 26087, 26119, |
| | 24775 | 24999, 25031, | 25383, 25415, | 25767, 25799, | 26151, 26183, |
| | | 25063, 25095, | 25447, 25479, | 25831, 25863, | 26215, 26247, |
| | | 25127, 25159 | 25511, 25543 | 25895, 25927 | 26279, 26311 |
| 24648 | 24680, | 24808, 24840, | 25192, 25224, | 25576, 25608, | 25960, 25992, |
| | 24712, | 24872, 24904, | 25256, 25288, | 25640, 25672, | 26024, 26056, |
| | 24744, | 24936, 24968, | 25320, 25352, | 25704, 25736, | 26088, 26120, |
| | 24776 | 25000, 25032, | 25384, 25416, | 25768, 25800, | 26152, 26184, |
| | | 25064, 25096, | 25448, 25480, | 25832, 25864, | 26216, 26248, |
| | | 25128, 25160 | 25512, 25544 | 25896, 25928 | 26280, 26312 |
| 24649 | 24681, | 24809, 24841, | 25193, 25225, | 25577, 25609, | 25961, 25993, |
| | 24713, | 24873, 24905, | 25257, 25289, | 25641, 25673, | 26025, 26057, |
| | 24745, | 24937, 24969, | 25321, 25353, | 25705, 25737, | 26089, 26121, |
| | 24777 | 25001, 25033, | 25385, 25417, | 25769, 25801, | 26153, 26185, |
| | | 25065, 25097, | 25449, 25481, | 25833, 25865, | 26217, 26249, |
| | | 25129, 25161 | 25513, 25545 | 25897, 25929 | 26281, 26313 |
| 24650 | 24682, | 24810, 24842, | 25194, 25226, | 25578, 25610, | 25962, 25994, |
| | 24714, | 24874, 24906, | 25258, 25290, | 25642, 25674, | 26026, 26058, |
| | 24746, | 24938, 24970, | 25322, 25354, | 25706, 25738, | 26090, 26122, |
| | 24778 | 25002, 25034, | 25386, 25418, | 25770, 25802, | 26154, 26186, |
| | | 25066, 25098, | 25450, 25482, | 25834, 25866, | 26218, 26250, |
| | | 25130, 25162 | 25514, 25546 | 25898, 25930 | 26282, 26314 |
| 24651 | 24683, | 24811, 24843, | 25195, 25227, | 25579, 25611, | 25963, 25995, |
| | 24715, | 24875, 24907, | 25259, 25291, | 25643, 25675, | 26027, 26059, |
| | 24747, | 24939, 24971, | 25323, 25355, | 25707, 25739, | 26091, 26123, |
| | 24779 | 25003, 25035, | 25387, 25419, | 25771, 25803, | 26155, 26187, |
| | | 25067, 25099, | 25451, 25483, | 25835, 25867, | 26219, 26251, |
| | | 25131, 25163 | 25515, 25547 | 25899, 25931 | 26283, 26315 |
| 24652 | 24684, | 24812, 24844, | 25196, 25228, | 25580, 25612, | 25964, 25996, |
| | 24716, | 24876, 24908, | 25260, 25292, | 25644, 25676, | 26028, 26060, |
| | 24748, | 24940, 24972, | 25324, 25356, | 25708, 25740, | 26092, 26124, |
| | 24780 | 25004, 25036, | 25388, 25420, | 25772, 25804, | 26156, 26188, |
| | | 25068, 25100, | 25452, 25484, | 25836, 25868, | 26220, 26252, |
| | | 25132, 25164 | 25516, 25548 | 25900, 25932 | 26284, 26316 |
| 24653 | 24685, | 24813, 24845, | 25197, 25229, | 25581, 25613, | 25965, 25997, |
| | 24717, | 24877, 24909, | 25261, 25293, | 25645, 25677, | 26029, 26061, |
| | 24749, | 24941, 24973, | 25325, 25357, | 25709, 25741, | 26093, 26125, |
| | 24781 | 25005, 25037, | 25389, 25421, | 25773, 25805, | 26157, 26189, |
| | | 25069, 25101, | 25453, 25485, | 25837, 25869, | 26221, 26253, |
| | | 25133, 25165 | 25517, 25549 | 25901, 25933 | 26285, 26317 |
| 24654 | 24686, | 24814, 24846, | 25198, 25230, | 25582, 25614, | 25966, 25998, |
| | 24718, | 24878, 24910, | 25262, 25294, | 25646, 25678, | 26030, 26062, |
| | 24750, | 24942, 24974, | 25326, 25358, | 25710, 25742, | 26094, 26126, |
| | 24782 | 25006, 25038, | 25390, 25422, | 25774, 25806, | 26158, 26190, |
| | | 25070, 25102, | 25454, 25486, | 25838, 25870, | 26222, 26254, |
| | | 25134, 25166 | 25518, 25550 | 25902, 25934 | 26286, 26318 |
| 24655 | 24687, | 24815, 24847, | 25199, 25231, | 25583, 25615, | 25967, 25999, |
| | 24719, | 24879, 24911, | 25263, 25295, | 25647, 25679, | 26031, 26063, |
| | 24751, | 24943, 24975, | 25327, 25359, | 25711, 25743, | 26095, 26127, |
| | 24783 | 25007, 25039, | 25391, 25423, | 25775, 25807, | 26159, 26191, |
| | | 25071, 25103, | 25455, 25487, | 25839, 25871, | 26223, 26255, |
| | | 25135, 25167 | 25519, 25551 | 25903, 25935 | 26287, 26319 |
| 24656 | 24688, | 24816, 24848, | 25200, 25232, | 25584, 25616, | 25968, 26000, |
| | 24720, | 24880, 24912, | 25264, 25296, | 25648, 25680, | 26032, 26064, |
| | 24752, | 24944, 24976, | 25328, 25360, | 25712, 25744, | 26096, 26128, |
| | 24784 | 25008, 25040, | 25392, 25424, | 25776, 25808, | 26160, 26192, |
| | | 25072, 25104, | 25456, 25488, | 25840, 25872, | 26224, 26256, |
| | | 25136, 25168 | 25520, 25552 | 25904, 25936 | 26288, 26320 |
| 24657 | 24689, | 24817, 24849, | 25201, 25233, | 25585, 25617, | 25969, 26001, |
| | 24721, | 24881, 24913, | 25265, 25297, | 25649, 25681, | 26033, 26065, |
| | 24753, | 24945, 24977, | 25329, 25361, | 25713, 25745, | 26097, 26129, |
| | 24785 | 25009, 25041, | 25393, 25425, | 25777, 25809, | 26161, 26193, |
| | | 25073, 25105, | 25457, 25489, | 25841, 25873, | 26225, 26257, |
| | | 25137, 25169 | 25521, 25553 | 25905, 25937 | 26289, 26321 |
| 24658 | 24690, | 24818, 24850, | 25202, 25234, | 25586, 25618, | 25970, 26002, |
| | 24722, | 24882, 24914, | 25266, 25298, | 25650, 25682, | 26034, 26066, |
| | 24754, | 24946, 24978, | 25330, 25362, | 25714, 25746, | 26098, 26130, |
| | 24786 | 25010, 25042, | 25394, 25426, | 25778, 25810, | 26162, 26194, |
| | | 25074, 25106, | 25458, 25490, | 25842, 25874, | 26226, 26258, |
| | | 25138, 25170 | 25522, 25554 | 25906, 25938 | 26290, 26322 |
| 24659 | 24691, | 24819, 24851, | 25203, 25235, | 25587, 25619, | 25971, 26003, |
| | 24723, | 24883, 24915, | 25267, 25299, | 25651, 25683, | 26035, 26067, |
| | 24755, | 24947, 24979, | 25331, 25363, | 25715, 25747, | 26099, 26131, |
| | 24787 | 25011, 25043, | 25395, 25427, | 25779, 25811, | 26163, 26195, |
| | | 25075, 25107, | 25459, 25491, | 25843, 25875, | 26227, 26259, |
| | | 25139, 25171 | 25523, 25555 | 25907, 25939 | 26291, 26323 |
| 24660 | 24692, | 24820, 24852, | 25204, 25236, | 25588, 25620, | 25972, 26004, |
| | 24724, | 24884, 24916, | 25268, 25300, | 25652, 25684, | 26036, 26068, |
| | 24756, | 24948, 24980, | 25332, 25364, | 25716, 25748, | 26100, 26132, |
| | 24788 | 25012, 25044, | 25396, 25428, | 25780, 25812, | 26164, 26196, |
| | | 25076, 25108, | 25460, 25492, | 25844, 25876, | 26228, 26260, |
| | | 25140, 25172 | 25524, 25556 | 25908, 25940 | 26292, 26324 |
| 24661 | 24693, | 24821, 24853, | 25205, 25237, | 25589, 25621, | 25973, 26005, |
| | 24725, | 24885, 24917, | 25269, 25301, | 25653, 25685, | 26037, 26069, |
| | 24757, | 24949, 24981, | 25333, 25365, | 25717, 25749, | 26101, 26133, |
| | 24789 | 25013, 25045, | 25397, 25429, | 25781, 25813, | 26165, 26197, |
| | | 25077, 25109, | 25461, 25493, | 25845, 25877, | 26229, 26261, |
| | | 25141, 25173 | 25525, 25557 | 25909, 25941 | 26293, 26325 |
| 24662 | 24694, | 24822, 24854, | 25206, 25238, | 25590, 25622, | 25974, 26006, |
| | 24726, | 24886, 24918, | 25270, 25302, | 25654, 25686, | 26038, 26070, |
| | 24758, | 24950, 24982, | 25334, 25366, | 25718, 25750, | 26102, 26134, |
| | 24790 | 25014, 25046, | 25398, 25430, | 25782, 25814, | 26166, 26198, |
| | | 25078, 25110, | 25462, 25494, | 25846, 25878, | 26230, 26262, |
| | | 25142, 25174 | 25526, 25558 | 25910, 25942 | 26294, 26326 |
| 24663 | 24695, | 24823, 24855, | 25207, 25239, | 25591, 25623, | 25975, 26007, |
| | 24727, | 24887, 24919, | 25271, 25303, | 25655, 25687, | 26039, 26071, |
| | 24759, | 24951, 24983, | 25335, 25367, | 25719, 25751, | 26103, 26135, |
| | 24791 | 25015, 25047, | 25399, 25431, | 25783, 25815, | 26167, 26199, |
| | | 25079, 25111, | 25463, 25495, | 25847, 25879, | 26231, 26263, |
| | | 25143, 25175 | 25527, 25559 | 25911, 25943 | 26295, 26327 |
| 24664 | 24696, | 24824, 24856, | 25208, 25240, | 25592, 25624, | 25976, 26008, |
| | 24728, | 24888, 24920, | 25272, 25304, | 25656, 25688, | 26040, 26072, |
| | 24760, | 24952, 24984, | 25336, 25368, | 25720, 25752, | 26104, 26136, |
| | 24792 | 25016, 25048, | 25400, 25432, | 25784, 25816, | 26168, 26200, |
| | | 25080, 25112, | 25464, 25496, | 25848, 25880, | 26232, 26264, |
| | | 25144, 25176 | 25528, 25560 | 25912, 25944 | 26296, 26328 |
| 24665 | 24697, | 24825, 24857, | 25209, 25241, | 25593, 25625, | 25977, 26009, |
| | 24729, | 24889, 24921, | 25273, 25305, | 25657, 25689, | 26041, 26073, |
| | 24761, | 24953, 24985, | 25337, 25369, | 25721, 25753, | 26105, 26137, |
| | 24793 | 25017, 25049, | 25401, 25433, | 25785, 25817, | 26169, 26201, |
| | | 25081, 25113, | 25465, 25497, | 25849, 25881, | 26233, 26265, |
| | | 25145, 25177 | 25529, 25561 | 25913, 25945 | 26297, 26329 |
| 24666 | 24698, | 24826, 24858, | 25210, 25242, | 25594, 25626, | 25978, 26010, |
| | 24730, | 24890, 24922, | 25274, 25306, | 25658, 25690, | 26042, 26074, |
| | 24762, | 24954, 24986, | 25338, 25370, | 25722, 25754, | 26106, 26138, |
| | 24794 | 25018, 25050, | 25402, 25434, | 25786, 25818, | 26170, 26202, |
| | | 25082, 25114, | 25466, 25498, | 25850, 25882, | 26234, 26266, |
| | | 25146, 25178 | 25530, 25562 | 25914, 25946 | 26298, 26330 |
| 24667 | 24699, | 24827, 24859, | 25211, 25243, | 25595, 25627, | 25979, 26011, |
| | 24731, | 24891, 24923, | 25275, 25307, | 25659, 25691, | 26043, 26075, |
| | 24763, | 24955, 24987, | 25339, 25371, | 25723, 25755, | 26107, 26139, |
| | 24795 | 25019, 25051, | 25403, 25435, | 25787, 25819, | 26171, 26203, |
| | | 25083, 25115, | 25467, 25499, | 25851, 25883, | 26235, 26267, |
| | | 25147, 25179 | 25531, 25563 | 25915, 25947 | 26299, 26331 |
| 24668 | 24700, | 24828, 24860, | 25212, 25244, | 25596, 25628, | 25980, 26012, |
| | 24732, | 24892, 24924, | 25276, 25308, | 25660, 25692, | 26044, 26076, |
| | 24764, | 24956, 24988, | 25340, 25372, | 25724, 25756, | 26108, 26140, |
| | 24796 | 25020, 25052, | 25404, 25436, | 25788, 25820, | 26172, 26204, |
| | | 25084, 25116, | 25468, 25500, | 25852, 25884, | 26236, 26268, |
| | | 25148, 25180 | 25532, 25564 | 25916, 25948 | 26300, 26332 |
| 24669 | 24701, | 24829, 24861, | 25213, 25245, | 25597, 25629, | 25981, 26013, |
| | 24733, | 24893, 24925, | 25277, 25309, | 25661, 25693, | 26045, 26077, |
| | 24765, | 24957, 24989, | 25341, 25373, | 25725, 25757, | 26109, 26141, |
| | 24797 | 25021, 25053, | 25405, 25437, | 25789, 25821, | 26173, 26205, |
| | | 25085, 25117, | 25469, 25501, | 25853, 25885, | 26237, 26269, |
| | | 25149, 25181 | 25533, 25565 | 25917, 25949 | 26301, 26333 |
| 24670 | 24702, | 24830, 24862, | 25214, 25246, | 25598, 25630, | 25982, 26014, |
| | 24734, | 24894, 24926, | 25278, 25310, | 25662, 25694, | 26046, 26078, |
| | 24766, | 24958, 24990, | 25342, 25374, | 25726, 25758, | 26110, 26142, |
| | 24798 | 25022, 25054, | 25406, 25438, | 25790, 25822, | 26174, 26206, |
| | | 25086, 25118, | 25470, 25502, | 25854, 25886, | 26238, 26270, |
| | | 25150, 25182 | 25534, 25566 | 25918, 25950 | 26302, 26334 |
| 24671 | 24703, | 24831, 24863, | 25215, 25247, | 25599, 25631, | 25983, 26015, |
| | 24735, | 24895, 24927, | 25279, 25311, | 25663, 25695, | 26047, 26079, |
| | 24767, | 24959, 24991, | 25343, 25375, | 25727, 25759, | 26111, 26143, |
| | 24799 | 25023, 25055, | 25407, 25439, | 25791, 25823, | 26175, 26207, |
| | | 25087, 25119, | 25471, 25503, | 25855, 25887, | 26239, 26271, |
| | | 25151, 25183 | 25535, 25567 | 25919, 25951 | 26303, 26335 |
| 24672 | 24704, | 24832, 24864, | 25216, 25248, | 25600, 25632, | 25984, 26016, |
| | 24736, | 24896, 24928, | 25280, 25312, | 25664, 25696, | 26048, 26080, |
| | 24768, | 24960, 24992, | 25344, 25376, | 25728, 25760, | 26112, 26144, |
| | 24800 | 25024, 25056, | 25408, 25440, | 25792, 25824, | 26176, 26208, |
| | | 25088, 25120, | 25472, 25504, | 25856, 25888, | 26240, 26272, |
| | | 25152, 25184 | 25536, 25568 | 25920, 25952 | 26304, 26336 |
| 24673 | 24705, | 24833, 24865, | 25217, 25249, | 25601, 25633, | 25985, 26017, |
| | 24737, | 24897, 24929, | 25281, 25313, | 25665, 25697, | 26049, 26081, |
| | 24769, | 24961, 24993, | 25345, 25377, | 25729, 25761, | 26113, 26145, |
| | 24801 | 25025, 25057, | 25409, 25441, | 25793, 25825, | 26177, 26209, |
| | | 25089, 25121, | 25473, 25505, | 25857, 25889, | 26241, 26273, |
| | | 25153, 25185 | 25537, 25569 | 25921, 25953 | 26305, 26337 |
| 24674 | 24706, | 24834, 24866, | 25218, 25250, | 25602, 25634, | 25986, 26018, |
| | 24738, | 24898, 24930, | 25282, 25314, | 25666, 25698, | 26050, 26082, |
| | 24770, | 24962, 24994, | 25346, 25378, | 25730, 25762, | 26114, 26146, |
| | 24802 | 25026, 25058, | 25410, 25442, | 25794, 25826, | 26178, 26210, |
| | | 25090, 25122, | 25474, 25506, | 25858, 25890, | 26242, 26274, |
| | | 25154, 25186 | 25538, 25570 | 25922, 25954 | 26306, 26338 |
| 24675 | 24707, | 24835, 24867, | 25219, 25251, | 25603, 25635, | 25987, 26019, |
| | 24739, | 24899, 24931, | 25283, 25315, | 25667, 25699, | 26051, 26083, |
| | 24771, | 24963, 24995, | 25347, 25379, | 25731, 25763, | 26115, 26147, |
| | 24803 | 25027, 25059, | 25411, 25443, | 25795, 25827, | 26179, 26211, |
| | | 25091, 25123, | 25475, 25507, | 25859, 25891, | 26243, 26275, |
| | | 25155, 25187 | 25539, 25571 | 25923, 25955 | 26307, 26339 |
| 24676 | 24708, | 24836, 24868, | 25220, 25252, | 25604, 25636, | 25988, 26020, |
| | 24740, | 24900, 24932, | 25284, 25316, | 25668, 25700, | 26052, 26084, |
| | 24772, | 24964, 24996, | 25348, 25380, | 25732, 25764, | 26116, 26148, |
| | 24804 | 25028, 25060, | 25412, 25444, | 25796, 25828, | 26180, 26212, |
| | | 25092, 25124, | 25476, 25508, | 25860, 25892, | 26244, 26276, |
| | | 25156, 25188 | 25540, 25572 | 25924, 25956 | 26308, 26340 |

TABLE 3.4
Preferred LASV prefusion-stabilized (GPCmut4) constructs

| | | RNA designs and SEQ ID NOs | | | | |
|---|---|---|---|---|---|---|
| PRT | CDS | a-1 | a-2 | b-4 | i-2 | i-3 |

| | | | | | |
|---|---|---|---|---|---|
| 26341 | 26373, | 26501, 26533, | 26885, 26917, | 27269, 27301, | 27653, 27685, |
| | 26405, | 26565, 26597, | 26949, 26981, | 27333, 27365, | 27717, 27749, |
| | 26437, | 26629, 26661, | 27013, 27045, | 27397, 27429, | 27781, 27813, |
| | 26469 | 26693, 26725, | 27077, 27109, | 27461, 27493, | 27845, 27877, |
| | | 26757, 26789, | 27141, 27173, | 27525, 27557, | 27909, 27941, |
| | | 26821, 26853 | 27205, 27237 | 27589, 27621 | 27973, 28005 |
| 26342 | 26374, | 26502, 26534, | 26886, 26918, | 27270, 27302, | 27654, 27686, |
| | 26406, | 26566, 26598, | 26950, 26982, | 27334, 27366, | 27718, 27750, |
| | 26438, | 26630, 26662, | 27014, 27046, | 27398, 27430, | 27782, 27814, |
| | 26470 | 26694, 26726, | 27078, 27110, | 27462, 27494, | 27846, 27878, |
| | | 26758, 26790, | 27142, 27174, | 27526, 27558, | 27910, 27942, |
| | | 26822, 26854 | 27206, 27238 | 27590, 27622 | 27974, 28006 |
| 26343 | 26375, | 26503, 26535, | 26887, 26919, | 27271, 27303, | 27655, 27687, |
| | 26407, | 26567, 26599, | 26951, 26983, | 27335, 27367, | 27719, 27751, |
| | 26439, | 26631, 26663, | 27015, 27047, | 27399, 27431, | 27783, 27815, |
| | 26471 | 26695, 26727, | 27079, 27111, | 27463, 27495, | 27847, 27879, |
| | | 26759, 26791, | 27143, 27175, | 27527, 27559, | 27911, 27943, |
| | | 26823, 26855 | 27207, 27239 | 27591, 27623 | 27975, 28007 |
| 26344 | 26376, | 26504, 26536, | 26888, 26920, | 27272, 27304, | 27656, 27688, |
| | 26408, | 26568, 26600, | 26952, 26984, | 27336, 27368, | 27720, 27752, |
| | 26440, | 26632, 26664, | 27016, 27048, | 27400, 27432, | 27784, 27816, |
| | 26472 | 26696, 26728, | 27080, 27112, | 27464, 27496, | 27848, 27880, |
| | | 26760, 26792, | 27144, 27176, | 27528, 27560, | 27912, 27944, |
| | | 26824, 26856 | 27208, 27240 | 27592, 27624 | 27976, 28008 |
| 26345 | 26377, | 26505, 26537, | 26889, 26921, | 27273, 27305, | 27657, 27689, |
| | 26409, | 26569, 26601, | 26953, 26985, | 27337, 27369, | 27721, 27753, |
| | 26441, | 26633, 26665, | 27017, 27049, | 27401, 27433, | 27785, 27817, |
| | 26473 | 26697, 26729, | 27081, 27113, | 27465, 27497, | 27849, 27881, |
| | | 26761, 26793, | 27145, 27177, | 27529, 27561, | 27913, 27945, |
| | | 26825, 26857 | 27209, 27241 | 27593, 27625 | 27977, 28009 |
| 26346 | 26378, | 26506, 26538, | 26890, 26922, | 27274, 27306, | 27658, 27690, |
| | 26410, | 26570, 26602, | 26954, 26986, | 27338, 27370, | 27722, 27754, |
| | 26442, | 26634, 26666, | 27018, 27050, | 27402, 27434, | 27786, 27818, |
| | 26474 | 26698, 26730, | 27082, 27114, | 27466, 27498, | 27850, 27882, |
| | | 26762, 26794, | 27146, 27178, | 27530, 27562, | 27914, 27946, |
| | | 26826, 26858 | 27210, 27242 | 27594, 27626 | 27978, 28010 |
| 26347 | 26379, | 26507, 26539, | 26891, 26923, | 27275, 27307, | 27659, 27691, |
| | 26411, | 26571, 26603, | 26955, 26987, | 27339, 27371, | 27723, 27755, |
| | 26443, | 26635, 26667, | 27019, 27051, | 27403, 27435, | 27787, 27819, |
| | 26475 | 26699, 26731, | 27083, 27115, | 27467, 27499, | 27851, 27883, |
| | | 26763, 26795, | 27147, 27179, | 27531, 27563, | 27915, 27947, |
| | | 26827, 26859 | 27211, 27243 | 27595, 27627 | 27979, 28011 |
| 26348 | 26380, | 26508, 26540, | 26892, 26924, | 27276, 27308, | 27660, 27692, |
| | 26412, | 26572, 26604, | 26956, 26988, | 27340, 27372, | 27724, 27756, |
| | 26444, | 26636, 26668, | 27020, 27052, | 27404, 27436, | 27788, 27820, |
| | 26476 | 26700, 26732, | 27084, 27116, | 27468, 27500, | 27852, 27884, |
| | | 26764, 26796, | 27148, 27180, | 27532, 27564, | 27916, 27948, |
| | | 26828, 26860 | 27212, 27244 | 27596, 27628 | 27980, 28012 |
| 26349 | 26381, | 26509, 26541, | 26893, 26925, | 27277, 27309, | 27661, 27693, |
| | 26413, | 26573, 26605, | 26957, 26989, | 27341, 27373, | 27725, 27757, |
| | 26445, | 26637, 26669, | 27021, 27053, | 27405, 27437, | 27789, 27821, |
| | 26477 | 26701, 26733, | 27085, 27117, | 27469, 27501, | 27853, 27885, |
| | | 26765, 26797, | 27149, 27181, | 27533, 27565, | 27917, 27949, |
| | | 26829, 26861 | 27213, 27245 | 27597, 27629 | 27981, 28013 |
| 26350 | 26382, | 26510, 26542, | 26894, 26926, | 27278, 27310, | 27662, 27694, |
| | 26414, | 26574, 26606, | 26958, 26990, | 27342, 27374, | 27726, 27758, |
| | 26446, | 26638, 26670, | 27022, 27054, | 27406, 27438, | 27790, 27822, |
| | 26478 | 26702, 26734, | 27086, 27118, | 27470, 27502, | 27854, 27886, |
| | | 26766, 26798, | 27150, 27182, | 27534, 27566, | 27918, 27950, |
| | | 26830, 26862 | 27214, 27246 | 27598, 27630 | 27982, 28014 |
| 26351 | 26383, | 26511, 26543, | 26895, 26927, | 27279, 27311, | 27663, 27695, |
| | 26415, | 26575, 26607, | 26959, 26991, | 27343, 27375, | 27727, 27759, |
| | 26447, | 26639, 26671, | 27023, 27055, | 27407, 27439, | 27791, 27823, |
| | 26479 | 26703, 26735, | 27087, 27119, | 27471, 27503, | 27855, 27887, |
| | | 26767, 26799, | 27151, 27183, | 27535, 27567, | 27919, 27951, |
| | | 26831, 26863 | 27215, 27247 | 27599, 27631 | 27983, 28015 |
| 26352 | 26384, | 26512, 26544, | 26896, 26928, | 27280, 27312, | 27664, 27696, |
| | 26416, | 26576, 26608, | 26960, 26992, | 27344, 27376, | 27728, 27760, |
| | 26448, | 26640, 26672, | 27024, 27056, | 27408, 27440, | 27792, 27824, |
| | 26480 | 26704, 26736, | 27088, 27120, | 27472, 27504, | 27856, 27888, |
| | | 26768, 26800, | 27152, 27184, | 27536, 27568, | 27920, 27952, |
| | | 26832, 26864 | 27216, 27248 | 27600, 27632 | 27984, 28016 |
| 26353 | 26385, | 26513, 26545, | 26897, 26929, | 27281, 27313, | 27665, 27697, |
| | 26417, | 26577, 26609, | 26961, 26993, | 27345, 27377, | 27729, 27761, |
| | 26449, | 26641, 26673, | 27025, 27057, | 27409, 27441, | 27793, 27825, |
| | 26481 | 26705, 26737, | 27089, 27121, | 27473, 27505, | 27857, 27889, |
| | | 26769, 26801, | 27153, 27185, | 27537, 27569, | 27921, 27953, |
| | | 26833, 26865 | 27217, 27249 | 27601, 27633 | 27985, 28017 |
| 26354 | 26386, | 26514, 26546, | 26898, 26930, | 27282, 27314, | 27666, 27698, |
| | 26418, | 26578, 26610, | 26962, 26994, | 27346, 27378, | 27730, 27762, |
| | 26450, | 26642, 26674, | 27026, 27058, | 27410, 27442, | 27794, 27826, |
| | 26482 | 26706, 26738, | 27090, 27122, | 27474, 27506, | 27858, 27890, |
| | | 26770, 26802, | 27154, 27186, | 27538, 27570, | 27922, 27954, |
| | | 26834, 26866 | 27218, 27250 | 27602, 27634 | 27986, 28018 |
| 26355 | 26387, | 26515, 26547, | 26899, 26931, | 27283, 27315, | 27667, 27699, |
| | 26419, | 26579, 26611, | 26963, 26995, | 27347, 27379, | 27731, 27763, |
| | 26451, | 26643, 26675, | 27027, 27059, | 27411, 27443, | 27795, 27827, |
| | 26483 | 26707, 26739, | 27091, 27123, | 27475, 27507, | 27859, 27891, |
| | | 26771, 26803, | 27155, 27187, | 27539, 27571, | 27923, 27955, |
| | | 26835, 26867 | 27219, 27251 | 27603, 27635 | 27987, 28019 |
| 26356 | 26388, | 26516, 26548, | 26900, 26932, | 27284, 27316, | 27668, 27700, |
| | 26420, | 26580, 26612, | 26964, 26996, | 27348, 27380, | 27732, 27764, |
| | 26452, | 26644, 26676, | 27028, 27060, | 27412, 27444, | 27796, 27828, |
| | 26484 | 26708, 26740, | 27092, 27124, | 27476, 27508, | 27860, 27892, |
| | | 26772, 26804, | 27156, 27188, | 27540, 27572, | 27924, 27956, |
| | | 26836, 26868 | 27220, 27252 | 27604, 27636 | 27988, 28020 |
| 26357 | 26389, | 26517, 26549, | 26901, 26933, | 27285, 27317, | 27669, 27701, |
| | 26421, | 26581, 26613, | 26965, 26997, | 27349, 27381, | 27733, 27765, |
| | 26453, | 26645, 26677, | 27029, 27061, | 27413, 27445, | 27797, 27829, |
| | 26485 | 26709, 26741, | 27093, 27125, | 27477, 27509, | 27861, 27893, |
| | | 26773, 26805, | 27157, 27189, | 27541, 27573, | 27925, 27957, |
| | | 26837, 26869 | 27221, 27253 | 27605, 27637 | 27989, 28021 |
| 26358 | 26390, | 26518, 26550, | 26902, 26934, | 27286, 27318, | 27670, 27702, |
| | 26422, | 26582, 26614, | 26966, 26998, | 27350, 27382, | 27734, 27766, |
| | 26454, | 26646, 26678, | 27030, 27062, | 27414, 27446, | 27798, 27830, |
| | 26486 | 26710, 26742, | 27094, 27126, | 27478, 27510, | 27862, 27894, |
| | | 26774, 26806, | 27158, 27190, | 27542, 27574, | 27926, 27958, |
| | | 26838, 26870 | 27222, 27254 | 27606, 27638 | 27990, 28022 |
| 26359 | 26391, | 26519, 26551, | 26903, 26935, | 27287, 27319, | 27671, 27703, |
| | 26423, | 26583, 26615, | 26967, 26999, | 27351, 27383, | 27735, 27767, |
| | 26455, | 26647, 26679, | 27031, 27063, | 27415, 27447, | 27799, 27831, |
| | 26487 | 26711, 26743, | 27095, 27127, | 27479, 27511, | 27863, 27895, |
| | | 26775, 26807, | 27159, 27191, | 27543, 27575, | 27927, 27959, |
| | | 26839, 26871 | 27223, 27255 | 27607, 27639 | 27991, 28023 |
| 26360 | 26392, | 26520, 26552, | 26904, 26936, | 27288, 27320, | 27672, 27704, |
| | 26424, | 26584, 26616, | 26968, 27000, | 27352, 27384, | 27736, 27768, |
| | 26456, | 26648, 26680, | 27032, 27064, | 27416, 27448, | 27800, 27832, |
| | 26488 | 26712, 26744, | 27096, 27128, | 27480, 27512, | 27864, 27896, |
| | | 26776, 26808, | 27160, 27192, | 27544, 27576, | 27928, 27960, |
| | | 26840, 26872 | 27224, 27256 | 27608, 27640 | 27992, 28024 |
| 26361 | 26393, | 26521, 26553, | 26905, 26937, | 27289, 27321, | 27673, 27705, |
| | 26425, | 26585, 26617, | 26969, 27001, | 27353, 27385, | 27737, 27769, |
| | 26457, | 26649, 26681, | 27033, 27065, | 27417, 27449, | 27801, 27833, |
| | 26489 | 26713, 26745, | 27097, 27129, | 27481, 27513, | 27865, 27897, |
| | | 26777, 26809, | 27161, 27193, | 27545, 27577, | 27929, 27961, |
| | | 26841, 26873 | 27225, 27257 | 27609, 27641 | 27993, 28025 |
| 26362 | 26394, | 26522, 26554, | 26906, 26938, | 27290, 27322, | 27674, 27706, |
| | 26426, | 26586, 26618, | 26970, 27002, | 27354, 27386, | 27738, 27770, |
| | 26458, | 26650, 26682, | 27034, 27066, | 27418, 27450, | 27802, 27834, |
| | 26490 | 26714, 26746, | 27098, 27130, | 27482, 27514, | 27866, 27898, |
| | | 26778, 26810, | 27162, 27194, | 27546, 27578, | 27930, 27962, |
| | | 26842, 26874 | 27226, 27258 | 27610, 27642 | 27994, 28026 |
| 26363 | 26395, | 26523, 26555, | 26907, 26939, | 27291, 27323, | 27675, 27707, |
| | 26427, | 26587, 26619, | 26971, 27003, | 27355, 27387, | 27739, 27771, |
| | 26459, | 26651, 26683, | 27035, 27067, | 27419, 27451, | 27803, 27835, |
| | 26491 | 26715, 26747, | 27099, 27131, | 27483, 27515, | 27867, 27899, |
| | | 26779, 26811, | 27163, 27195, | 27547, 27579, | 27931, 27963, |
| | | 26843, 26875 | 27227, 27259 | 27611, 27643 | 27995, 28027 |
| 26364 | 26396, | 26524, 26556, | 26908, 26940, | 27292, 27324, | 27676, 27708, |
| | 26428, | 26588, 26620, | 26972, 27004, | 27356, 27388, | 27740, 27772, |
| | 26460, | 26652, 26684, | 27036, 27068, | 27420, 27452, | 27804, 27836, |
| | 26492 | 26716, 26748, | 27100, 27132, | 27484, 27516, | 27868, 27900, |
| | | 26780, 26812, | 27164, 27196, | 27548, 27580, | 27932, 27964, |
| | | 26844, 26876 | 27228, 27260 | 27612, 27644 | 27996, 28028 |
| 26365 | 26397, | 26525, 26557, | 26909, 26941, | 27293, 27325, | 27677, 27709, |
| | 26429, | 26589, 26621, | 26973, 27005, | 27357, 27389, | 27741, 27773, |
| | 26461, | 26653, 26685, | 27037, 27069, | 27421, 27453, | 27805, 27837, |
| | 26493 | 26717, 26749, | 27101, 27133, | 27485, 27517, | 27869, 27901, |
| | | 26781, 26813, | 27165, 27197, | 27549, 27581, | 27933, 27965, |
| | | 26845, 26877 | 27229, 27261 | 27613, 27645 | 27997, 28029 |
| 26366 | 26398, | 26526, 26558, | 26910, 26942, | 27294, 27326, | 27678, 27710, |
| | 26430, | 26590, 26622, | 26974, 27006, | 27358, 27390, | 27742, 27774, |
| | 26462, | 26654, 26686, | 27038, 27070, | 27422, 27454, | 27806, 27838, |
| | 26494 | 26718, 26750, | 27102, 27134, | 27486, 27518, | 27870, 27902, |
| | | 26782, 26814, | 27166, 27198, | 27550, 27582, | 27934, 27966, |
| | | 26846, 26878 | 27230, 27262 | 27614, 27646 | 27998, 28030 |
| 26367 | 26399, | 26527, 26559, | 26911, 26943, | 27295, 27327, | 27679, 27711, |
| | 26431, | 26591, 26623, | 26975, 27007, | 27359, 27391, | 27743, 27775, |
| | 26463, | 26655, 26687, | 27039, 27071, | 27423, 27455, | 27807, 27839, |
| | 26495 | 26719, 26751, | 27103, 27135, | 27487, 27519, | 27871, 27903, |
| | | 26783, 26815, | 27167, 27199, | 27551, 27583, | 27935, 27967, |
| | | 26847, 26879 | 27231, 27263 | 27615, 27647 | 27999, 28031 |
| 26368 | 26400, | 26528, 26560, | 26912, 26944, | 27296, 27328, | 27680, 27712, |
| | 26432, | 26592, 26624, | 26976, 27008, | 27360, 27392, | 27744, 27776, |
| | 26464, | 26656, 26688, | 27040, 27072, | 27424, 27456, | 27808, 27840, |
| | 26496 | 26720, 26752, | 27104, 27136, | 27488, 27520, | 27872, 27904, |
| | | 26784, 26816, | 27168, 27200, | 27552, 27584, | 27936, 27968, |
| | | 26848, 26880 | 27232, 27264 | 27616, 27648 | 28000, 28032 |
| 26369 | 26401, | 26529, 26561, | 26913, 26945, | 27297, 27329, | 27681, 27713, |
| | 26433, | 26593, 26625, | 26977, 27009, | 27361, 27393, | 27745, 27777, |
| | 26465, | 26657, 26689, | 27041, 27073, | 27425, 27457, | 27809, 27841, |
| | 26497 | 26721, 26753, | 27105, 27137, | 27489, 27521, | 27873, 27905, |
| | | 26785, 26817, | 27169, 27201, | 27553, 27585, | 27937, 27969, |
| | | 26849, 26881 | 27233, 27265 | 27617, 27649 | 28001, 28033 |
| 26370 | 26402, | 26530, 26562, | 26914, 26946, | 27298, 27330, | 27682, 27714, |
| | 26434, | 26594, 26626, | 26978, 27010, | 27362, 27394, | 27746, 27778, |
| | 26466, | 26658, 26690, | 27042, 27074, | 27426, 27458, | 27810, 27842, |
| | 26498 | 26722, 26754, | 27106, 27138, | 27490, 27522, | 27874, 27906, |
| | | 26786, 26818, | 27170, 27202, | 27554, 27586, | 27938, 27970, |
| | | 26850, 26882 | 27234, 27266 | 27618, 27650 | 28002, 28034 |
| 26371 | 26403, | 26531, 26563, | 26915, 26947, | 27299, 27331, | 27683, 27715, |
| | 26435, | 26595, 26627, | 26979, 27011, | 27363, 27395, | 27747, 27779, |
| | 26467, | 26659, 26691, | 27043, 27075, | 27427, 27459, | 27811, 27843, |
| | 26499 | 26723, 26755, | 27107, 27139, | 27491, 27523, | 27875, 27907, |
| | | 26787, 26819, | 27171, 27203, | 27555, 27587, | 27939, 27971, |
| | | 26851, 26883 | 27235, 27267 | 27619, 27651 | 28003, 28035 |
| 26372 | 26404, | 26532, 26564, | 26916, 26948, | 27300, 27332, | 27684, 27716, |
| | 26436, | 26596, 26628, | 26980, 27012, | 27364, 27396, | 27748, 27780, |
| | 26468, | 26660, 26692, | 27044, 27076, | 27428, 27460, | 27812, 27844, |
| | 26500 | 26724, 26756, | 27108, 27140, | 27492, 27524, | 27876, 27908, |
| | | 26788, 26820, | 27172, 27204, | 27556, 27588, | 27940, 27972, |
| | | 26852, 26884 | 27236, 27268 | 27620, 27652 | 28004, 28036 |

TABLE 3.5
Preferred LASV prefusion-stabilized (GPCmut5) constructs

| | | RNA designs and SEQ ID NOs | | | | |
|---|---|---|---|---|---|---|
| PRT | CDS | a-1 | a-2 | b-4 | i-2 | i-3 |

| | | | | | |
|---|---|---|---|---|---|
| 28037 | 28069, | 28197, 28229, | 28581, 28613, | 28965, 28997, | 29349, 29381, |
| | 28101, | 28261, 28293, | 28645, 28677, | 29029, 29061, | 29413, 29445, |
| | 28133, | 28325, 28357, | 28709, 28741, | 29093, 29125, | 29477, 29509, |
| | 28165 | 28389, 28421, | 28773, 28805, | 29157, 29189, | 29541, 29573, |
| | | 28453, 28485, | 28837, 28869, | 29221, 29253, | 29605, 29637, |
| | | 28517, 28549 | 28901, 28933 | 29285, 29317 | 29669, 29701 |
| 28038 | 28070, | 28198, 28230, | 28582, 28614, | 28966, 28998, | 29350, 29382, |
| | 28102, | 28262, 28294, | 28646, 28678, | 29030, 29062, | 29414, 29446, |
| | 28134, | 28326, 28358, | 28710, 28742, | 29094, 29126, | 29478, 29510, |
| | 28166 | 28390, 28422, | 28774, 28806, | 29158, 29190, | 29542, 29574, |
| | | 28454, 28486, | 28838, 28870, | 29222, 29254, | 29606, 29638, |
| | | 28518, 28550 | 28902, 28934 | 29286, 29318 | 29670, 29702 |
| 28039 | 28071, | 28199, 28231, | 28583, 28615, | 28967, 28999, | 29351, 29383, |
| | 28103, | 28263, 28295, | 28647, 28679, | 29031, 29063, | 29415, 29447, |
| | 28135, | 28327, 28359, | 28711, 28743, | 29095, 29127, | 29479, 29511, |
| | 28167 | 28391, 28423, | 28775, 28807, | 29159, 29191, | 29543, 29575, |
| | | 28455, 28487, | 28839, 28871, | 29223, 29255, | 29607, 29639, |
| | | 28519, 28551 | 28903, 28935 | 29287, 29319 | 29671, 29703 |
| 28040 | 28072, | 28200, 28232, | 28584, 28616, | 28968, 29000, | 29352, 29384, |
| | 28104, | 28264, 28296, | 28648, 28680, | 29032, 29064, | 29416, 29448, |
| | 28136, | 28328, 28360, | 28712, 28744, | 29096, 29128, | 29480, 29512, |
| | 28168 | 28392, 28424, | 28776, 28808, | 29160, 29192, | 29544, 29576, |
| | | 28456, 28488, | 28840, 28872, | 29224, 29256, | 29608, 29640, |
| | | 28520, 28552 | 28904, 28936 | 29288, 29320 | 29672, 29704 |
| 28041 | 28073, | 28201, 28233, | 28585, 28617, | 28969, 29001, | 29353, 29385, |
| | 28105, | 28265, 28297, | 28649, 28681, | 29033, 29065, | 29417, 29449, |
| | 28137, | 28329, 28361, | 28713, 28745, | 29097, 29129, | 29481, 29513, |
| | 28169 | 28393, 28425, | 28777, 28809, | 29161, 29193, | 29545, 29577, |
| | | 28457, 28489, | 28841, 28873, | 29225, 29257, | 29609, 29641, |
| | | 28521, 28553 | 28905, 28937 | 29289, 29321 | 29673, 29705 |
| 28042 | 28074, | 28202, 28234, | 28586, 28618, | 28970, 29002, | 29354, 29386, |
| | 28106, | 28266, 28298, | 28650, 28682, | 29034, 29066, | 29418, 29450, |
| | 28138, | 28330, 28362, | 28714, 28746, | 29098, 29130, | 29482, 29514, |
| | 28170 | 28394, 28426, | 28778, 28810, | 29162, 29194, | 29546, 29578, |
| | | 28458, 28490, | 28842, 28874, | 29226, 29258, | 29610, 29642, |
| | | 28522, 28554 | 28906, 28938 | 29290, 29322 | 29674, 29706 |
| 28043 | 28075, | 28203, 28235, | 28587, 28619, | 28971, 29003, | 29355, 29387, |
| | 28107, | 28267, 28299, | 28651, 28683, | 29035, 29067, | 29419, 29451, |
| | 28139, | 28331, 28363, | 28715, 28747, | 29099, 29131, | 29483, 29515, |
| | 28171 | 28395, 28427, | 28779, 28811, | 29163, 29195, | 29547, 29579, |
| | | 28459, 28491, | 28843, 28875, | 29227, 29259, | 29611, 29643, |
| | | 28523, 28555 | 28907, 28939 | 29291, 29323 | 29675, 29707 |
| 28044 | 28076, | 28204, 28236, | 28588, 28620, | 28972, 29004, | 29356, 29388, |
| | 28108, | 28268, 28300, | 28652, 28684, | 29036, 29068, | 29420, 29452, |
| | 28140, | 28332, 28364, | 28716, 28748, | 29100, 29132, | 29484, 29516, |
| | 28172 | 28396, 28428, | 28780, 28812, | 29164, 29196, | 29548, 29580, |
| | | 28460, 28492, | 28844, 28876, | 29228, 29260, | 29612, 29644, |
| | | 28524, 28556 | 28908, 28940 | 29292, 29324 | 29676, 29708 |
| 28045 | 28077, | 28205, 28237, | 28589, 28621, | 28973, 29005, | 29357, 29389, |
| | 28109, | 28269, 28301, | 28653, 28685, | 29037, 29069, | 29421, 29453, |
| | 28141, | 28333, 28365, | 28717, 28749, | 29101, 29133, | 29485, 29517, |
| | 28173 | 28397, 28429, | 28781, 28813, | 29165, 29197, | 29549, 29581, |
| | | 28461, 28493, | 28845, 28877, | 29229, 29261, | 29613, 29645, |
| | | 28525, 28557 | 28909, 28941 | 29293, 29325 | 29677, 29709 |
| 28046 | 28078, | 28206, 28238, | 28590, 28622, | 28974, 29006, | 29358, 29390, |
| | 28110, | 28270, 28302, | 28654, 28686, | 29038, 29070, | 29422, 29454, |
| | 28142, | 28334, 28366, | 28718, 28750, | 29102, 29134, | 29486, 29518, |
| | 28174 | 28398, 28430, | 28782, 28814, | 29166, 29198, | 29550, 29582, |
| | | 28462, 28494, | 28846, 28878, | 29230, 29262, | 29614, 29646, |
| | | 28526, 28558 | 28910, 28942 | 29294, 29326 | 29678, 29710 |
| 28047 | 28079, | 28207, 28239, | 28591, 28623, | 28975, 29007, | 29359, 29391, |
| | 28111, | 28271, 28303, | 28655, 28687, | 29039, 29071, | 29423, 29455, |
| | 28143, | 28335, 28367, | 28719, 28751, | 29103, 29135, | 29487, 29519, |
| | 28175 | 28399, 28431, | 28783, 28815, | 29167, 29199, | 29551, 29583, |
| | | 28463, 28495, | 28847, 28879, | 29231, 29263, | 29615, 29647, |
| | | 28527, 28559 | 28911, 28943 | 29295, 29327 | 29679, 29711 |
| 28048 | 28080, | 28208, 28240, | 28592, 28624, | 28976, 29008, | 29360, 29392, |
| | 28112, | 28272, 28304, | 28656, 28688, | 29040, 29072, | 29424, 29456, |
| | 28144, | 28336, 28368, | 28720, 28752, | 29104, 29136, | 29488, 29520, |
| | 28176 | 28400, 28432, | 28784, 28816, | 29168, 29200, | 29552, 29584, |
| | | 28464, 28496, | 28848, 28880, | 29232, 29264, | 29616, 29648, |
| | | 28528, 28560 | 28912, 28944 | 29296, 29328 | 29680, 29712 |
| 28049 | 28081, | 28209, 28241, | 28593, 28625, | 28977, 29009, | 29361, 29393, |
| | 28113, | 28273, 28305, | 28657, 28689, | 29041, 29073, | 29425, 29457, |
| | 28145, | 28337, 28369, | 28721, 28753, | 29105, 29137, | 29489, 29521, |
| | 28177 | 28401, 28433, | 28785, 28817, | 29169, 29201, | 29553, 29585, |
| | | 28465, 28497, | 28849, 28881, | 29233, 29265, | 29617, 29649, |
| | | 28529, 28561 | 28913, 28945 | 29297, 29329 | 29681, 29713 |
| 28050 | 28082, | 28210, 28242, | 28594, 28626, | 28978, 29010, | 29362, 29394, |
| | 28114, | 28274, 28306, | 28658, 28690, | 29042, 29074, | 29426, 29458, |
| | 28146, | 28338, 28370, | 28722, 28754, | 29106, 29138, | 29490, 29522, |
| | 28178 | 28402, 28434, | 28786, 28818, | 29170, 29202, | 29554, 29586, |
| | | 28466, 28498, | 28850, 28882, | 29234, 29266, | 29618, 29650, |
| | | 28530, 28562 | 28914, 28946 | 29298, 29330 | 29682, 29714 |
| 28051 | 28083, | 28211, 28243, | 28595, 28627, | 28979, 29011, | 29363, 29395, |
| | 28115, | 28275, 28307, | 28659, 28691, | 29043, 29075, | 29427, 29459, |
| | 28147, | 28339, 28371, | 28723, 28755, | 29107, 29139, | 29491, 29523, |
| | 28179 | 28403, 28435, | 28787, 28819, | 29171, 29203, | 29555, 29587, |
| | | 28467, 28499, | 28851, 28883, | 29235, 29267, | 29619, 29651, |
| | | 28531, 28563 | 28915, 28947 | 29299, 29331 | 29683, 29715 |
| 28052 | 28084, | 28212, 28244, | 28596, 28628, | 28980, 29012, | 29364, 29396, |
| | 28116, | 28276, 28308, | 28660, 28692, | 29044, 29076, | 29428, 29460, |
| | 28148, | 28340, 28372, | 28724, 28756, | 29108, 29140, | 29492, 29524, |
| | 28180 | 28404, 28436, | 28788, 28820, | 29172, 29204, | 29556, 29588, |
| | | 28468, 28500, | 28852, 28884, | 29236, 29268, | 29620, 29652, |
| | | 28532, 28564 | 28916, 28948 | 29300, 29332 | 29684, 29716 |
| 28053 | 28085, | 28213, 28245, | 28597, 28629, | 28981, 29013, | 29365, 29397, |
| | 28117, | 28277, 28309, | 28661, 28693, | 29045, 29077, | 29429, 29461, |
| | 28149, | 28341, 28373, | 28725, 28757, | 29109, 29141, | 29493, 29525, |
| | 28181 | 28405, 28437, | 28789, 28821, | 29173, 29205, | 29557, 29589, |
| | | 28469, 28501, | 28853, 28885, | 29237, 29269, | 29621, 29653, |
| | | 28533, 28565 | 28917, 28949 | 29301, 29333 | 29685, 29717 |
| 28054 | 28086, | 28214, 28246, | 28598, 28630, | 28982, 29014, | 29366, 29398, |
| | 28118, | 28278, 28310, | 28662, 28694, | 29046, 29078, | 29430, 29462, |
| | 28150, | 28342, 28374, | 28726, 28758, | 29110, 29142, | 29494, 29526, |
| | 28182 | 28406, 28438, | 28790, 28822, | 29174, 29206, | 29558, 29590, |
| | | 28470, 28502, | 28854, 28886, | 29238, 29270, | 29622, 29654, |
| | | 28534, 28566 | 28918, 28950 | 29302, 29334 | 29686, 29718 |
| 28055 | 28087, | 28215, 28247, | 28599, 28631, | 28983, 29015, | 29367, 29399, |
| | 28119, | 28279, 28311, | 28663, 28695, | 29047, 29079, | 29431, 29463, |
| | 28151, | 28343, 28375, | 28727, 28759, | 29111, 29143, | 29495, 29527, |
| | 28183 | 28407, 28439, | 28791, 28823, | 29175, 29207, | 29559, 29591, |
| | | 28471, 28503, | 28855, 28887, | 29239, 29271, | 29623, 29655, |
| | | 28535, 28567 | 28919, 28951 | 29303, 29335 | 29687, 29719 |
| 28056 | 28088, | 28216, 28248, | 28600, 28632, | 28984, 29016, | 29368, 29400, |
| | 28120, | 28280, 28312, | 28664, 28696, | 29048, 29080, | 29432, 29464, |
| | 28152, | 28344, 28376, | 28728, 28760, | 29112, 29144, | 29496, 29528, |
| | 28184 | 28408, 28440, | 28792, 28824, | 29176, 29208, | 29560, 29592, |
| | | 28472, 28504, | 28856, 28888, | 29240, 29272, | 29624, 29656, |
| | | 28536, 28568 | 28920, 28952 | 29304, 29336 | 29688, 29720 |
| 28057 | 28089, | 28217, 28249, | 28601, 28633, | 28985, 29017, | 29369, 29401, |
| | 28121, | 28281, 28313, | 28665, 28697, | 29049, 29081, | 29433, 29465, |
| | 28153, | 28345, 28377, | 28729, 28761, | 29113, 29145, | 29497, 29529, |
| | 28185 | 28409, 28441, | 28793, 28825, | 29177, 29209, | 29561, 29593, |
| | | 28473, 28505, | 28857, 28889, | 29241, 29273, | 29625, 29657, |
| | | 28537, 28569 | 28921, 28953 | 29305, 29337 | 29689, 29721 |
| 28058 | 28090, | 28218, 28250, | 28602, 28634, | 28986, 29018, | 29370, 29402, |
| | 28122, | 28282, 28314, | 28666, 28698, | 29050, 29082, | 29434, 29466, |
| | 28154, | 28346, 28378, | 28730, 28762, | 29114, 29146, | 29498, 29530, |
| | 28186 | 28410, 28442, | 28794, 28826, | 29178, 29210, | 29562, 29594, |
| | | 28474, 28506, | 28858, 28890, | 29242, 29274, | 29626, 29658, |
| | | 28538, 28570 | 28922, 28954 | 29306, 29338 | 29690, 29722 |
| 28059 | 28091, | 28219, 28251, | 28603, 28635, | 28987, 29019, | 29371, 29403, |
| | 28123, | 28283, 28315, | 28667, 28699, | 29051, 29083, | 29435, 29467, |
| | 28155, | 28347, 28379, | 28731, 28763, | 29115, 29147, | 29499, 29531, |
| | 28187 | 28411, 28443, | 28795, 28827, | 29179, 29211, | 29563, 29595, |
| | | 28475, 28507, | 28859, 28891, | 29243, 29275, | 29627, 29659, |
| | | 28539, 28571 | 28923, 28955 | 29307, 29339 | 29691, 29723 |
| 28060 | 28092, | 28220, 28252, | 28604, 28636, | 28988, 29020, | 29372, 29404, |
| | 28124, | 28284, 28316, | 28668, 28700, | 29052, 29084, | 29436, 29468, |
| | 28156, | 28348, 28380, | 28732, 28764, | 29116, 29148, | 29500, 29532, |
| | 28188 | 28412, 28444, | 28796, 28828, | 29180, 29212, | 29564, 29596, |
| | | 28476, 28508, | 28860, 28892, | 29244, 29276, | 29628, 29660, |
| | | 28540, 28572 | 28924, 28956 | 29308, 29340 | 29692, 29724 |
| 28061 | 28093, | 28221, 28253, | 28605, 28637, | 28989, 29021, | 29373, 29405, |
| | 28125, | 28285, 28317, | 28669, 28701, | 29053, 29085, | 29437, 29469, |
| | 28157, | 28349, 28381, | 28733, 28765, | 29117, 29149, | 29501, 29533, |
| | 28189 | 28413, 28445, | 28797, 28829, | 29181, 29213, | 29565, 29597, |
| | | 28477, 28509, | 28861, 28893, | 29245, 29277, | 29629, 29661, |
| | | 28541, 28573 | 28925, 28957 | 29309, 29341 | 29693, 29725 |
| 28062 | 28094, | 28222, 28254, | 28606, 28638, | 28990, 29022, | 29374, 29406, |
| | 28126, | 28286, 28318, | 28670, 28702, | 29054, 29086, | 29438, 29470, |
| | 28158, | 28350, 28382, | 28734, 28766, | 29118, 29150, | 29502, 29534, |
| | 28190 | 28414, 28446, | 28798, 28830, | 29182, 29214, | 29566, 29598, |
| | | 28478, 28510, | 28862, 28894, | 29246, 29278, | 29630, 29662, |
| | | 28542, 28574 | 28926, 28958 | 29310, 29342 | 29694, 29726 |
| 28063 | 28095, | 28223, 28255, | 28607, 28639, | 28991, 29023, | 29375, 29407, |
| | 28127, | 28287, 28319, | 28671, 28703, | 29055, 29087, | 29439, 29471, |
| | 28159, | 28351, 28383, | 28735, 28767, | 29119, 29151, | 29503, 29535, |
| | 28191 | 28415, 28447, | 28799, 28831, | 29183, 29215, | 29567, 29599, |
| | | 28479, 28511, | 28863, 28895, | 29247, 29279, | 29631, 29663, |
| | | 28543, 28575 | 28927, 28959 | 29311, 29343 | 29695, 29727 |
| 28064 | 28096, | 28224, 28256, | 28608, 28640, | 28992, 29024, | 29376, 29408, |
| | 28128, | 28288, 28320, | 28672, 28704, | 29056, 29088, | 29440, 29472, |
| | 28160, | 28352, 28384, | 28736, 28768, | 29120, 29152, | 29504, 29536, |
| | 28192 | 28416, 28448, | 28800, 28832, | 29184, 29216, | 29568, 29600, |
| | | 28480, 28512, | 28864, 28896, | 29248, 29280, | 29632, 29664, |
| | | 28544, 28576 | 28928, 28960 | 29312, 29344 | 29696, 29728 |
| 28065 | 28097, | 28225, 28257, | 28609, 28641, | 28993, 29025, | 29377, 29409, |
| | 28129, | 28289, 28321, | 28673, 28705, | 29057, 29089, | 29441, 29473, |
| | 28161, | 28353, 28385, | 28737, 28769, | 29121, 29153, | 29505, 29537, |
| | 28193 | 28417, 28449, | 28801, 28833, | 29185, 29217, | 29569, 29601, |
| | | 28481, 28513, | 28865, 28897, | 29249, 29281, | 29633, 29665, |
| | | 28545, 28577 | 28929, 28961 | 29313, 29345 | 29697, 29729 |
| 28066 | 28098, | 28226, 28258, | 28610, 28642, | 28994, 29026, | 29378, 29410, |
| | 28130, | 28290, 28322, | 28674, 28706, | 29058, 29090, | 29442, 29474, |
| | 28162, | 28354, 28386, | 28738, 28770, | 29122, 29154, | 29506, 29538, |
| | 28194 | 28418, 28450, | 28802, 28834, | 29186, 29218, | 29570, 29602, |
| | | 28482, 28514, | 28866, 28898, | 29250, 29282, | 29634, 29666, |
| | | 28546, 28578 | 28930, 28962 | 29314, 29346 | 29698, 29730 |
| 28067 | 28099, | 28227, 28259, | 28611, 28643, | 28995, 29027, | 29379, 29411, |
| | 28131, | 28291, 28323, | 28675, 28707, | 29059, 29091, | 29443, 29475, |
| | 28163, | 28355, 28387, | 28739, 28771, | 29123, 29155, | 29507, 29539, |
| | 28195 | 28419, 28451, | 28803, 28835, | 29187, 29219, | 29571, 29603, |
| | | 28483, 28515, | 28867, 28899, | 29251, 29283, | 29635, 29667, |
| | | 28547, 28579 | 28931, 28963 | 29315, 29347 | 29699, 29731 |
| 28068 | 28100, | 28228, 28260, | 28612, 28644, | 28996, 29028, | 29380, 29412, |
| | 28132, | 28292, 28324, | 28676, 28708, | 29060, 29092, | 29444, 29476, |
| | 28164, | 28356, 28388, | 28740, 28772, | 29124, 29156, | 29508, 29540, |
| | 28196 | 28420, 28452, | 28804, 28836, | 29188, 29220, | 29572, 29604, |
| | | 28484, 28516, | 28868, 28900, | 29252, 29284, | 29636, 29668, |
| | | 28548, 28580 | 28932, 28964 | 29316, 29348 | 29700, 29732 |

TABLE 3.6
Preferred LASV prefusion-stabilized (GPCmut6) constructs

| | | RNA designs and SEQ ID NOs | | | | |
|---|---|---|---|---|---|---|
| PRT | CDS | a-1 | a-2 | b-4 | i-2 | i-3 |
| 29733 | 29765, | 29893, 29925, | 30277, 30309, | 30661, 30693, | | 31045, 31077, |
| | 29797, | 29957, 29989, | 30341, 30373, | 30725, 30757, | | 31109, 31141, |
| | 29829, | 30021, 30053, | 30405, 30437, | 30789, 30821, | | 31173, 31205, |
| | 29861 | 30085, 30117, | 30469, 30501, | 30853, 30885, | | 31237, 31269, |
| | | 30149, 30181, | 30533, 30565, | 30917, 30949, | | 31301, 31333, |
| | | 30213, 30245 | 30597, 30629 | 30981, 31013 | | 31365, 31397 |
| 29734 | 29766, | 29894, 29926, | 30278, 30310, | 30662, 30694, | | 31046, 31078, |
| | 29798, | 29958, 29990, | 30342, 30374, | 30726, 30758, | | 31110, 31142, |
| | 29830, | 30022, 30054, | 30406, 30438, | 30790, 30822, | | 31174, 31206, |
| | 29862 | 30086, 30118, | 30470, 30502, | 30854, 30886, | | 31238, 31270, |
| | | 30150, 30182, | 30534, 30566, | 30918, 30950, | | 31302, 31334, |
| | | 30214, 30246 | 30598, 30630 | 30982, 31014 | | 31366, 31398 |
| 29735 | 29767, | 29895, 29927, | 30279, 30311, | 30663, 30695, | | 31047, 31079, |
| | 29799, | 29959, 29991, | 30343, 30375, | 30727, 30759, | | 31111, 31143, |
| | 29831, | 30023, 30055, | 30407, 30439, | 30791, 30823, | | 31175, 31207, |
| | 29863 | 30087, 30119, | 30471, 30503, | 30855, 30887, | | 31239, 31271, |
| | | 30151, 30183, | 30535, 30567, | 30919, 30951, | | 31303, 31335, |
| | | 30215, 30247 | 30599, 30631 | 30983, 31015 | | 31367, 31399 |
| 29736 | 29768, | 29896, 29928, | 30280, 30312, | 30664, 30696, | | 31048, 31080, |
| | 29800, | 29960, 29992, | 30344, 30376, | 30728, 30760, | | 31112, 31144, |
| | 29832, | 30024, 30056, | 30408, 30440, | 30792, 30824, | | 31176, 31208, |
| | 29864 | 30088, 30120, | 30472, 30504, | 30856, 30888, | | 31240, 31272, |
| | | 30152, 30184, | 30536, 30568, | 30920, 30952, | | 31304, 31336, |
| | | 30216, 30248 | 30600, 30632 | 30984, 31016 | | 31368, 31400 |
| 29737 | 29769, | 29897, 29929, | 30281, 30313, | 30665, 30697, | | 31049, 31081, |
| | 29801, | 29961, 29993, | 30345, 30377, | 30729, 30761, | | 31113, 31145, |
| | 29833, | 30025, 30057, | 30409, 30441, | 30793, 30825, | | 31177, 31209, |
| | 29865 | 30089, 30121, | 30473, 30505, | 30857, 30889, | | 31241, 31273, |
| | | 30153, 30185, | 30537, 30569, | 30921, 30953, | | 31305, 31337, |
| | | 30217, 30249 | 30601, 30633 | 30985, 31017 | | 31369, 31401 |
| 29738 | 29770, | 29898, 29930, | 30282, 30314, | 30666, 30698, | | 31050, 31082, |
| | 29802, | 29962, 29994, | 30346, 30378, | 30730, 30762, | | 31114, 31146, |
| | 29834, | 30026, 30058, | 30410, 30442, | 30794, 30826, | | 31178, 31210, |
| | 29866 | 30090, 30122, | 30474, 30506, | 30858, 30890, | | 31242, 31274, |
| | | 30154, 30186, | 30538, 30570, | 30922, 30954, | | 31306, 31338, |
| | | 30218, 30250 | 30602, 30634 | 30986, 31018 | | 31370, 31402 |
| 29739 | 29771, | 29899, 29931, | 30283, 30315, | 30667, 30699, | | 31051, 31083, |
| | 29803, | 29963, 29995, | 30347, 30379, | 30731, 30763, | | 31115, 31147, |
| | 29835, | 30027, 30059, | 30411, 30443, | 30795, 30827, | | 31179, 31211, |
| | 29867 | 30091, 30123, | 30475, 30507, | 30859, 30891, | | 31243, 31275, |
| | | 30155, 30187, | 30539, 30571, | 30923, 30955, | | 31307, 31339, |
| | | 30219, 30251 | 30603, 30635 | 30987, 31019 | | 31371, 31403 |
| 29740 | 29772, | 29900, 29932, | 30284, 30316, | 30668, 30700, | | 31052, 31084, |
| | 29804, | 29964, 29996, | 30348, 30380, | 30732, 30764, | | 31116, 31148, |
| | 29836, | 30028, 30060, | 30412, 30444, | 30796, 30828, | | 31180, 31212, |
| | 29868 | 30092, 30124, | 30476, 30508, | 30860, 30892, | | 31244, 31276, |
| | | 30156, 30188, | 30540, 30572, | 30924, 30956, | | 31308, 31340, |
| | | 30220, 30252 | 30604, 30636 | 30988, 31020 | | 31372, 31404 |
| 29741 | 29773, | 29901, 29933, | 30285, 30317, | 30669, 30701, | | 31053, 31085, |
| | 29805, | 29965, 29997, | 30349, 30381, | 30733, 30765, | | 31117, 31149, |
| | 29837, | 30029, 30061, | 30413, 30445, | 30797, 30829, | | 31181, 31213, |
| | 29869 | 30093, 30125, | 30477, 30509, | 30861, 30893, | | 31245, 31277, |
| | | 30157, 30189, | 30541, 30573, | 30925, 30957, | | 31309, 31341, |
| | | 30221, 30253 | 30605, 30637 | 30989, 31021 | | 31373, 31405 |
| 29742 | 29774, | 29902, 29934, | 30286, 30318, | 30670, 30702, | | 31054, 31086, |
| | 29806, | 29966, 29998, | 30350, 30382, | 30734, 30766, | | 31118, 31150, |
| | 29838, | 30030, 30062, | 30414, 30446, | 30798, 30830, | | 31182, 31214, |
| | 29870 | 30094, 30126, | 30478, 30510, | 30862, 30894, | | 31246, 31278, |
| | | 30158, 30190, | 30542, 30574, | 30926, 30958, | | 31310, 31342, |
| | | 30222, 30254 | 30606, 30638 | 30990, 31022 | | 31374, 31406 |
| 29743 | 29775, | 29903, 29935, | 30287, 30319, | 30671, 30703, | | 31055, 31087, |
| | 29807, | 29967, 29999, | 30351, 30383, | 30735, 30767, | | 31119, 31151, |
| | 29839, | 30031, 30063, | 30415, 30447, | 30799, 30831, | | 31183, 31215, |
| | 29871 | 30095, 30127, | 30479, 30511, | 30863, 30895, | | 31247, 31279, |
| | | 30159, 30191, | 30543, 30575, | 30927, 30959, | | 31311, 31343, |
| | | 30223, 30255 | 30607, 30639 | 30991, 31023 | | 31375, 31407 |
| 29744 | 29776, | 29904, 29936, | 30288, 30320, | 30672, 30704, | | 31056, 31088, |
| | 29808, | 29968, 30000, | 30352, 30384, | 30736, 30768, | | 31120, 31152, |
| | 29840, | 30032, 30064, | 30416, 30448, | 30800, 30832, | | 31184, 31216, |
| | 29872 | 30096, 30128, | 30480, 30512, | 30864, 30896, | | 31248, 31280, |
| | | 30160, 30192, | 30544, 30576, | 30928, 30960, | | 31312, 31344, |
| | | 30224, 30256 | 30608, 30640 | 30992, 31024 | | 31376, 31408 |
| 29745 | 29777, | 29905, 29937, | 30289, 30321, | 30673, 30705, | | 31057, 31089, |
| | 29809, | 29969, 30001, | 30353, 30385, | 30737, 30769, | | 31121, 31153, |
| | 29841, | 30033, 30065, | 30417, 30449, | 30801, 30833, | | 31185, 31217, |
| | 29873 | 30097, 30129, | 30481, 30513, | 30865, 30897, | | 31249, 31281, |
| | | 30161, 30193, | 30545, 30577, | 30929, 30961, | | 31313, 31345, |
| | | 30225, 30257 | 30609, 30641 | 30993, 31025 | | 31377, 31409 |
| 29746 | 29778, | 29906, 29938, | 30290, 30322, | 30674, 30706, | | 31058, 31090, |
| | 29810, | 29970, 30002, | 30354, 30386, | 30738, 30770, | | 31122, 31154, |
| | 29842, | 30034, 30066, | 30418, 30450, | 30802, 30834, | | 31186, 31218, |
| | 29874 | 30098, 30130, | 30482, 30514, | 30866, 30898, | | 31250, 31282, |
| | | 30162, 30194, | 30546, 30578, | 30930, 30962, | | 31314, 31346, |
| | | 30226, 30258 | 30610, 30642 | 30994, 31026 | | 31378, 31410 |
| 29747 | 29779, | 29907, 29939, | 30291, 30323, | 30675, 30707, | | 31059, 31091, |
| | 29811, | 29971, 30003, | 30355, 30387, | 30739, 30771, | | 31123, 31155, |
| | 29843, | 30035, 30067, | 30419, 30451, | 30803, 30835, | | 31187, 31219, |
| | 29875 | 30099, 30131, | 30483, 30515, | 30867, 30899, | | 31251, 31283, |
| | | 30163, 30195, | 30547, 30579, | 30931, 30963, | | 31315, 31347, |
| | | 30227, 30259 | 30611, 30643 | 30995, 31027 | | 31379, 31411 |
| 29748 | 29780, | 29908, 29940, | 30292, 30324, | 30676, 30708, | | 31060, 31092, |
| | 29812, | 29972, 30004, | 30356, 30388, | 30740, 30772, | | 31124, 31156, |
| | 29844, | 30036, 30068, | 30420, 30452, | 30804, 30836, | | 31188, 31220, |
| | 29876 | 30100, 30132, | 30484, 30516, | 30868, 30900, | | 31252, 31284, |
| | | 30164, 30196, | 30548, 30580, | 30932, 30964, | | 31316, 31348, |
| | | 30228, 30260 | 30612, 30644 | 30996, 31028 | | 31380, 31412 |
| 29749 | 29781, | 29909, 29941, | 30293, 30325, | 30677, 30709, | | 31061, 31093, |
| | 29813, | 29973, 30005, | 30357, 30389, | 30741, 30773, | | 31125, 31157, |
| | 29845, | 30037, 30069, | 30421, 30453, | 30805, 30837, | | 31189, 31221, |
| | 29877 | 30101, 30133, | 30485, 30517, | 30869, 30901, | | 31253, 31285, |
| | | 30165, 30197, | 30549, 30581, | 30933, 30965, | | 31317, 31349, |
| | | 30229, 30261 | 30613, 30645 | 30997, 31029 | | 31381, 31413 |
| 29750 | 29782, | 29910, 29942, | 30294, 30326, | 30678, 30710, | | 31062, 31094, |
| | 29814, | 29974, 30006, | 30358, 30390, | 30742, 30774, | | 31126, 31158, |
| | 29846, | 30038, 30070, | 30422, 30454, | 30806, 30838, | | 31190, 31222, |
| | 29878 | 30102, 30134, | 30486, 30518, | 30870, 30902, | | 31254, 31286, |
| | | 30166, 30198, | 30550, 30582, | 30934, 30966, | | 31318, 31350, |
| | | 30230, 30262 | 30614, 30646 | 30998, 31030 | | 31382, 31414 |
| 29751 | 29783, | 29911, 29943, | 30295, 30327, | 30679, 30711, | | 31063, 31095, |
| | 29815, | 29975, 30007, | 30359, 30391, | 30743, 30775, | | 31127, 31159, |
| | 29847, | 30039, 30071, | 30423, 30455, | 30807, 30839, | | 31191, 31223, |
| | 29879 | 30103, 30135, | 30487, 30519, | 30871, 30903, | | 31255, 31287, |
| | | 30167, 30199, | 30551, 30583, | 30935, 30967, | | 31319, 31351, |
| | | 30231, 30263 | 30615, 30647 | 30999, 31031 | | 31383, 31415 |
| 29752 | 29784, | 29912, 29944, | 30296, 30328, | 30680, 30712, | | 31064, 31096, |
| | 29816, | 29976, 30008, | 30360, 30392, | 30744, 30776, | | 31128, 31160, |
| | 29848, | 30040, 30072, | 30424, 30456, | 30808, 30840, | | 31192, 31224, |
| | 29880 | 30104, 30136, | 30488, 30520, | 30872, 30904, | | 31256, 31288, |
| | | 30168, 30200, | 30552, 30584, | 30936, 30968, | | 31320, 31352, |
| | | 30232, 30264 | 30616, 30648 | 31000, 31032 | | 31384, 31416 |
| 29753 | 29785, | 29913, 29945, | 30297, 30329, | 30681, 30713, | | 31065, 31097, |
| | 29817, | 29977, 30009, | 30361, 30393, | 30745, 30777, | | 31129, 31161, |
| | 29849, | 30041, 30073, | 30425, 30457, | 30809, 30841, | | 31193, 31225, |
| | 29881 | 30105, 30137, | 30489, 30521, | 30873, 30905, | | 31257, 31289, |
| | | 30169, 30201, | 30553, 30585, | 30937, 30969, | | 31321, 31353, |
| | | 30233, 30265 | 30617, 30649 | 31001, 31033 | | 31385, 31417 |
| 29754 | 29786, | 29914, 29946, | 30298, 30330, | 30682, 30714, | | 31066, 31098, |
| | 29818, | 29978, 30010, | 30362, 30394, | 30746, 30778, | | 31130, 31162, |
| | 29850, | 30042, 30074, | 30426, 30458, | 30810, 30842, | | 31194, 31226, |
| | 29882 | 30106, 30138, | 30490, 30522, | 30874, 30906, | | 31258, 31290, |
| | | 30170, 30202, | 30554, 30586, | 30938, 30970, | | 31322, 31354, |
| | | 30234, 30266 | 30618, 30650 | 31002, 31034 | | 31386, 31418 |
| 29755 | 29787, | 29915, 29947, | 30299, 30331, | 30683, 30715, | | 31067, 31099, |
| | 29819, | 29979, 30011, | 30363, 30395, | 30747, 30779, | | 31131, 31163, |
| | 29851, | 30043, 30075, | 30427, 30459, | 30811, 30843, | | 31195, 31227, |
| | 29883 | 30107, 30139, | 30491, 30523, | 30875, 30907, | | 31259, 31291, |
| | | 30171, 30203, | 30555, 30587, | 30939, 30971, | | 31323, 31355, |
| | | 30235, 30267 | 30619, 30651 | 31003, 31035 | | 31387, 31419 |
| 29756 | 29788, | 29916, 29948, | 30300, 30332, | 30684, 30716, | | 31068, 31100, |
| | 29820, | 29980, 30012, | 30364, 30396, | 30748, 30780, | | 31132, 31164, |
| | 29852, | 30044, 30076, | 30428, 30460, | 30812, 30844, | | 31196, 31228, |
| | 29884 | 30108, 30140, | 30492, 30524, | 30876, 30908, | | 31260, 31292, |
| | | 30172, 30204, | 30556, 30588, | 30940, 30972, | | 31324, 31356, |
| | | 30236, 30268 | 30620, 30652 | 31004, 31036 | | 31388, 31420 |
| 29757 | 29789, | 29917, 29949, | 30301, 30333, | 30685, 30717, | | 31069, 31101, |
| | 29821, | 29981, 30013, | 30365, 30397, | 30749, 30781, | | 31133, 31165, |
| | 29853, | 30045, 30077, | 30429, 30461, | 30813, 30845, | | 31197, 31229, |
| | 29885 | 30109, 30141, | 30493, 30525, | 30877, 30909, | | 31261, 31293, |
| | | 30173, 30205, | 30557, 30589, | 30941, 30973, | | 31325, 31357, |
| | | 30237, 30269 | 30621, 30653 | 31005, 31037 | | 31389, 31421 |
| 29758 | 29790, | 29918, 29950, | 30302, 30334, | 30686, 30718, | | 31070, 31102, |
| | 29822, | 29982, 30014, | 30366, 30398, | 30750, 30782, | | 31134, 31166, |
| | 29854, | 30046, 30078, | 30430, 30462, | 30814, 30846, | | 31198, 31230, |
| | 29886 | 30110, 30142, | 30494, 30526, | 30878, 30910, | | 31262, 31294, |
| | | 30174, 30206, | 30558, 30590, | 30942, 30974, | | 31326, 31358, |
| | | 30238, 30270 | 30622, 30654 | 31006, 31038 | | 31390, 31422 |
| 29759 | 29791, | 29919, 29951, | 30303, 30335, | 30687, 30719, | | 31071, 31103, |
| | 29823, | 29983, 30015, | 30367, 30399, | 30751, 30783, | | 31135, 31167, |
| | 29855, | 30047, 30079, | 30431, 30463, | 30815, 30847, | | 31199, 31231, |
| | 29887 | 30111, 30143, | 30495, 30527, | 30879, 30911, | | 31263, 31295, |
| | | 30175, 30207, | 30559, 30591, | 30943, 30975, | | 31327, 31359, |
| | | 30239, 30271 | 30623, 30655 | 31007, 31039 | | 31391, 31423 |
| 29760 | 29792, | 29920, 29952, | 30304, 30336, | 30688, 30720, | | 31072, 31104, |
| | 29824, | 29984, 30016, | 30368, 30400, | 30752, 30784, | | 31136, 31168, |
| | 29856, | 30048, 30080, | 30432, 30464, | 30816, 30848, | | 31200, 31232, |
| | 29888 | 30112, 30144, | 30496, 30528, | 30880, 30912, | | 31264, 31296, |
| | | 30176, 30208, | 30560, 30592, | 30944, 30976, | | 31328, 31360, |
| | | 30240, 30272 | 30624, 30656 | 31008, 31040 | | 31392, 31424 |
| 29761 | 29793, | 29921, 29953, | 30305, 30337, | 30689, 30721, | | 31073, 31105, |
| | 29825, | 29985, 30017, | 30369, 30401, | 30753, 30785, | | 31137, 31169, |
| | 29857, | 30049, 30081, | 30433, 30465, | 30817, 30849, | | 31201, 31233, |
| | 29889 | 30113, 30145, | 30497, 30529, | 30881, 30913, | | 31265, 31297, |
| | | 30177, 30209, | 30561, 30593, | 30945, 30977, | | 31329, 31361, |
| | | 30241, 30273 | 30625, 30657 | 31009, 31041 | | 31393, 31425 |
| 29762 | 29794, | 29922, 29954, | 30306, 30338, | 30690, 30722, | | 31074, 31106, |
| | 29826, | 29986, 30018, | 30370, 30402, | 30754, 30786, | | 31138, 31170, |
| | 29858, | 30050, 30082, | 30434, 30466, | 30818, 30850, | | 31202, 31234, |
| | 29890 | 30114, 30146, | 30498, 30530, | 30882, 30914, | | 31266, 31298, |
| | | 30178, 30210, | 30562, 30594, | 30946, 30978, | | 31330, 31362, |
| | | 30242, 30274 | 30626, 30658 | 31010, 31042 | | 31394, 31426 |
| 29763 | 29795, | 29923, 29955, | 30307, 30339, | 30691, 30723, | | 31075, 31107, |
| | 29827, | 29987, 30019, | 30371, 30403, | 30755, 30787, | | 31139, 31171, |
| | 29859, | 30051, 30083, | 30435, 30467, | 30819, 30851, | | 31203, 31235, |
| | 29891 | 30115, 30147, | 30499, 30531, | 30883, 30915, | | 31267, 31299, |
| | | 30179, 30211, | 30563, 30595, | 30947, 30979, | | 31331, 31363, |
| | | 30243, 30275 | 30627, 30659 | 31011, 31043 | | 31395, 31427 |
| 29764 | 29796, | 29924, 29956, | 30308, 30340, | 30692, 30724, | | 31076, 31108, |
| | 29828, | 29988, 30020, | 30372, 30404, | 30756, 30788, | | 31140, 31172, |
| | 29860, | 30052, 30084, | 30436, 30468, | 30820, 30852, | | 31204, 31236, |
| | 29892 | 30116, 30148, | 30500, 30532, | 30884, 30916, | | 31268, 31300, |
| | | 30180, 30212, | 30564, 30596, | 30948, 30980, | | 31332, 31364, |
| | | 30244, 30276 | 30628, 30660 | 31012, 31044 | | 31396, 31428 |

TABLE 3.7
Preferred LASV prefusion-stabilized (GPCmut7) constructs

| | | RNA designs and SEQ ID NOs | | | | |
|---|---|---|---|---|---|---|
| PRT | CDS | a-1 | a-2 | b-4 | i-2 | i-3 |
| 31429 | 31461, | 31589, 31621, | 31973, 32005, | 32357, 32389, | | 32741, 32773, |
| | 31493, | 31653, 31685, | 32037, 32069, | 32421, 32453, | | 32805, 32837, |
| | 31525, | 31717, 31749, | 32101, 32133, | 32485, 32517, | | 32869, 32901, |
| | 31557 | 31781, 31813, | 32165, 32197, | 32549, 32581, | | 32933, 32965, |
| | | 31845, 31877, | 32229, 32261, | 32613, 32645, | | 32997, 33029, |
| | | 31909, 31941 | 32293, 32325 | 32677, 32709 | | 33061, 33093 |
| 31430 | 31462, | 31590, 31622, | 31974, 32006, | 32358, 32390, | | 32742, 32774, |
| | 31494, | 31654, 31686, | 32038, 32070, | 32422, 32454, | | 32806, 32838, |
| | 31526, | 31718, 31750, | 32102, 32134, | 32486, 32518, | | 32870, 32902, |
| | 31558 | 31782, 31814, | 32166, 32198, | 32550, 32582, | | 32934, 32966, |
| | | 31846, 31878, | 32230, 32262, | 32614, 32646, | | 32998, 33030, |
| | | 31910, 31942 | 32294, 32326 | 32678, 32710 | | 33062, 33094 |
| 31431 | 31463, | 31591, 31623, | 31975, 32007, | 32359, 32391, | | 32743, 32775, |
| | 31495, | 31655, 31687, | 32039, 32071, | 32423, 32455, | | 32807, 32839, |
| | 31527, | 31719, 31751, | 32103, 32135, | 32487, 32519, | | 32871, 32903, |
| | 31559 | 31783, 31815, | 32167, 32199, | 32551, 32583, | | 32935, 32967, |
| | | 31847, 31879, | 32231, 32263, | 32615, 32647, | | 32999, 33031, |
| | | 31911, 31943 | 32295, 32327 | 32679, 32711 | | 33063, 33095 |
| 31432 | 31464, | 31592, 31624, | 31976, 32008, | 32360, 32392, | | 32744, 32776, |
| | 31496, | 31656, 31688, | 32040, 32072, | 32424, 32456, | | 32808, 32840, |
| | 31528, | 31720, 31752, | 32104, 32136, | 32488, 32520, | | 32872, 32904, |
| | 31560 | 31784, 31816, | 32168, 32200, | 32552, 32584, | | 32936, 32968, |
| | | 31848, 31880, | 32232, 32264, | 32616, 32648, | | 33000, 33032, |
| | | 31912, 31944 | 32296, 32328 | 32680, 32712 | | 33064, 33096 |
| 31433 | 31465, | 31593, 31625, | 31977, 32009, | 32361, 32393, | | 32745, 32777, |
| | 31497, | 31657, 31689, | 32041, 32073, | 32425, 32457, | | 32809, 32841, |
| | 31529, | 31721, 31753, | 32105, 32137, | 32489, 32521, | | 32873, 32905, |
| | 31561 | 31785, 31817, | 32169, 32201, | 32553, 32585, | | 32937, 32969, |
| | | 31849, 31881, | 32233, 32265, | 32617, 32649, | | 33001, 33033, |
| | | 31913, 31945 | 32297, 32329 | 32681, 32713 | | 33065, 33097 |
| 31434 | 31466, | 31594, 31626, | 31978, 32010, | 32362, 32394, | | 32746, 32778, |
| | 31498, | 31658, 31690, | 32042, 32074, | 32426, 32458, | | 32810, 32842, |
| | 31530, | 31722, 31754, | 32106, 32138, | 32490, 32522, | | 32874, 32906, |
| | 31562 | 31786, 31818, | 32170, 32202, | 32554, 32586, | | 32938, 32970, |
| | | 31850, 31882, | 32234, 32266, | 32618, 32650, | | 33002, 33034, |
| | | 31914, 31946 | 32298, 32330 | 32682, 32714 | | 33066, 33098 |
| 31435 | 31467, | 31595, 31627, | 31979, 32011, | 32363, 32395, | | 32747, 32779, |
| | 31499, | 31659, 31691, | 32043, 32075, | 32427, 32459, | | 32811, 32843, |
| | 31531, | 31723, 31755, | 32107, 32139, | 32491, 32523, | | 32875, 32907, |
| | 31563 | 31787, 31819, | 32171, 32203, | 32555, 32587, | | 32939, 32971, |
| | | 31851, 31883, | 32235, 32267, | 32619, 32651, | | 33003, 33035, |
| | | 31915, 31947 | 32299, 32331 | 32683, 32715 | | 33067, 33099 |
| 31436 | 31468, | 31596, 31628, | 31980, 32012, | 32364, 32396, | | 32748, 32780, |
| | 31500, | 31660, 31692, | 32044, 32076, | 32428, 32460, | | 32812, 32844, |
| | 31532, | 31724, 31756, | 32108, 32140, | 32492, 32524, | | 32876, 32908, |
| | 31564 | 31788, 31820, | 32172, 32204, | 32556, 32588, | | 32940, 32972, |
| | | 31852, 31884, | 32236, 32268, | 32620, 32652, | | 33004, 33036, |
| | | 31916, 31948 | 32300, 32332 | 32684, 32716 | | 33068, 33100 |
| 31437 | 31469, | 31597, 31629, | 31981, 32013, | 32365, 32397, | | 32749, 32781, |
| | 31501, | 31661, 31693, | 32045, 32077, | 32429, 32461, | | 32813, 32845, |
| | 31533, | 31725, 31757, | 32109, 32141, | 32493, 32525, | | 32877, 32909, |
| | 31565 | 31789, 31821, | 32173, 32205, | 32557, 32589, | | 32941, 32973, |
| | | 31853, 31885, | 32237, 32269, | 32621, 32653, | | 33005, 33037, |
| | | 31917, 31949 | 32301, 32333 | 32685, 32717 | | 33069, 33101 |
| 31438 | 31470, | 31598, 31630, | 31982, 32014, | 32366, 32398, | | 32750, 32782, |
| | 31502, | 31662, 31694, | 32046, 32078, | 32430, 32462, | | 32814, 32846, |
| | 31534, | 31726, 31758, | 32110, 32142, | 32494, 32526, | | 32878, 32910, |
| | 31566 | 31790, 31822, | 32174, 32206, | 32558, 32590, | | 32942, 32974, |
| | | 31854, 31886, | 32238, 32270, | 32622, 32654, | | 33006, 33038, |
| | | 31918, 31950 | 32302, 32334 | 32686, 32718 | | 33070, 33102 |
| 31439 | 31471, | 31599, 31631, | 31983, 32015, | 32367, 32399, | | 32751, 32783, |
| | 31503, | 31663, 31695, | 32047, 32079, | 32431, 32463, | | 32815, 32847, |
| | 31535, | 31727, 31759, | 32111, 32143, | 32495, 32527, | | 32879, 32911, |
| | 31567 | 31791, 31823, | 32175, 32207, | 32559, 32591, | | 32943, 32975, |
| | | 31855, 31887, | 32239, 32271, | 32623, 32655, | | 33007, 33039, |
| | | 31919, 31951 | 32303, 32335 | 32687, 32719 | | 33071, 33103 |
| 31440 | 31472, | 31600, 31632, | 31984, 32016, | 32368, 32400, | | 32752, 32784, |
| | 31504, | 31664, 31696, | 32048, 32080, | 32432, 32464, | | 32816, 32848, |
| | 31536, | 31728, 31760, | 32112, 32144, | 32496, 32528, | | 32880, 32912, |
| | 31568 | 31792, 31824, | 32176, 32208, | 32560, 32592, | | 32944, 32976, |
| | | 31856, 31888, | 32240, 32272, | 32624, 32656, | | 33008, 33040, |
| | | 31920, 31952 | 32304, 32336 | 32688, 32720 | | 33072, 33104 |
| 31441 | 31473, | 31601, 31633, | 31985, 32017, | 32369, 32401, | | 32753, 32785, |
| | 31505, | 31665, 31697, | 32049, 32081, | 32433, 32465, | | 32817, 32849, |
| | 31537, | 31729, 31761, | 32113, 32145, | 32497, 32529, | | 32881, 32913, |
| | 31569 | 31793, 31825, | 32177, 32209, | 32561, 32593, | | 32945, 32977, |
| | | 31857, 31889, | 32241, 32273, | 32625, 32657, | | 33009, 33041, |
| | | 31921, 31953 | 32305, 32337 | 32689, 32721 | | 33073, 33105 |
| 31442 | 31474, | 31602, 31634, | 31986, 32018, | 32370, 32402, | | 32754, 32786, |
| | 31506, | 31666, 31698, | 32050, 32082, | 32434, 32466, | | 32818, 32850, |
| | 31538, | 31730, 31762, | 32114, 32146, | 32498, 32530, | | 32882, 32914, |
| | 31570 | 31794, 31826, | 32178, 32210, | 32562, 32594, | | 32946, 32978, |
| | | 31858, 31890, | 32242, 32274, | 32626, 32658, | | 33010, 33042, |
| | | 31922, 31954 | 32306, 32338 | 32690, 32722 | | 33074, 33106 |
| 31443 | 31475, | 31603, 31635, | 31987, 32019, | 32371, 32403, | | 32755, 32787, |
| | 31507, | 31667, 31699, | 32051, 32083, | 32435, 32467, | | 32819, 32851, |
| | 31539, | 31731, 31763, | 32115, 32147, | 32499, 32531, | | 32883, 32915, |
| | 31571 | 31795, 31827, | 32179, 32211, | 32563, 32595, | | 32947, 32979, |
| | | 31859, 31891, | 32243, 32275, | 32627, 32659, | | 33011, 33043, |
| | | 31923, 31955 | 32307, 32339 | 32691, 32723 | | 33075, 33107 |
| 31444 | 31476, | 31604, 31636, | 31988, 32020, | 32372, 32404, | | 32756, 32788, |
| | 31508, | 31668, 31700, | 32052, 32084, | 32436, 32468, | | 32820, 32852, |
| | 31540, | 31732, 31764, | 32116, 32148, | 32500, 32532, | | 32884, 32916, |
| | 31572 | 31796, 31828, | 32180, 32212, | 32564, 32596, | | 32948, 32980, |
| | | 31860, 31892, | 32244, 32276, | 32628, 32660, | | 33012, 33044, |
| | | 31924, 31956 | 32308, 32340 | 32692, 32724 | | 33076, 33108 |
| 31445 | 31477, | 31605, 31637, | 31989, 32021, | 32373, 32405, | | 32757, 32789, |
| | 31509, | 31669, 31701, | 32053, 32085, | 32437, 32469, | | 32821, 32853, |
| | 31541, | 31733, 31765, | 32117, 32149, | 32501, 32533, | | 32885, 32917, |
| | 31573 | 31797, 31829, | 32181, 32213, | 32565, 32597, | | 32949, 32981, |
| | | 31861, 31893, | 32245, 32277, | 32629, 32661, | | 33013, 33045, |
| | | 31925, 31957 | 32309, 32341 | 32693, 32725 | | 33077, 33109 |
| 31446 | 31478, | 31606, 31638, | 31990, 32022, | 32374, 32406, | | 32758, 32790, |
| | 31510, | 31670, 31702, | 32054, 32086, | 32438, 32470, | | 32822, 32854, |
| | 31542, | 31734, 31766, | 32118, 32150, | 32502, 32534, | | 32886, 32918, |
| | 31574 | 31798, 31830, | 32182, 32214, | 32566, 32598, | | 32950, 32982, |
| | | 31862, 31894, | 32246, 32278, | 32630, 32662, | | 33014, 33046, |
| | | 31926, 31958 | 32310, 32342 | 32694, 32726 | | 33078, 33110 |
| 31447 | 31479, | 31607, 31639, | 31991, 32023, | 32375, 32407, | | 32759, 32791, |
| | 31511, | 31671, 31703, | 32055, 32087, | 32439, 32471, | | 32823, 32855, |
| | 31543, | 31735, 31767, | 32119, 32151, | 32503, 32535, | | 32887, 32919, |
| | 31575 | 31799, 31831, | 32183, 32215, | 32567, 32599, | | 32951, 32983, |
| | | 31863, 31895, | 32247, 32279, | 32631, 32663, | | 33015, 33047, |
| | | 31927, 31959 | 32311, 32343 | 32695, 32727 | | 33079, 33111 |
| 31448 | 31480, | 31608, 31640, | 31992, 32024, | 32376, 32408, | | 32760, 32792, |
| | 31512, | 31672, 31704, | 32056, 32088, | 32440, 32472, | | 32824, 32856, |
| | 31544, | 31736, 31768, | 32120, 32152, | 32504, 32536, | | 32888, 32920, |
| | 31576 | 31800, 31832, | 32184, 32216, | 32568, 32600, | | 32952, 32984, |
| | | 31864, 31896, | 32248, 32280, | 32632, 32664, | | 33016, 33048, |
| | | 31928, 31960 | 32312, 32344 | 32696, 32728 | | 33080, 33112 |
| 31449 | 31481, | 31609, 31641, | 31993, 32025, | 32377, 32409, | | 32761, 32793, |
| | 31513, | 31673, 31705, | 32057, 32089, | 32441, 32473, | | 32825, 32857, |
| | 31545, | 31737, 31769, | 32121, 32153, | 32505, 32537, | | 32889, 32921, |
| | 31577 | 31801, 31833, | 32185, 32217, | 32569, 32601, | | 32953, 32985, |
| | | 31865, 31897, | 32249, 32281, | 32633, 32665, | | 33017, 33049, |
| | | 31929, 31961 | 32313, 32345 | 32697, 32729 | | 33081, 33113 |
| 31450 | 31482, | 31610, 31642, | 31994, 32026, | 32378, 32410, | | 32762, 32794, |
| | 31514, | 31674, 31706, | 32058, 32090, | 32442, 32474, | | 32826, 32858, |
| | 31546, | 31738, 31770, | 32122, 32154, | 32506, 32538, | | 32890, 32922, |
| | 31578 | 31802, 31834, | 32186, 32218, | 32570, 32602, | | 32954, 32986, |
| | | 31866, 31898, | 32250, 32282, | 32634, 32666, | | 33018, 33050, |
| | | 31930, 31962 | 32314, 32346 | 32698, 32730 | | 33082, 33114 |
| 31451 | 31483, | 31611, 31643, | 31995, 32027, | 32379, 32411, | | 32763, 32795, |
| | 31515, | 31675, 31707, | 32059, 32091, | 32443, 32475, | | 32827, 32859, |
| | 31547, | 31739, 31771, | 32123, 32155, | 32507, 32539, | | 32891, 32923, |
| | 31579 | 31803, 31835, | 32187, 32219, | 32571, 32603, | | 32955, 32987, |
| | | 31867, 31899, | 32251, 32283, | 32635, 32667, | | 33019, 33051, |
| | | 31931, 31963 | 32315, 32347 | 32699, 32731 | | 33083, 33115 |
| 31452 | 31484, | 31612, 31644, | 31996, 32028, | 32380, 32412, | | 32764, 32796, |
| | 31516, | 31676, 31708, | 32060, 32092, | 32444, 32476, | | 32828, 32860, |
| | 31548, | 31740, 31772, | 32124, 32156, | 32508, 32540, | | 32892, 32924, |
| | 31580 | 31804, 31836, | 32188, 32220, | 32572, 32604, | | 32956, 32988, |
| | | 31868, 31900, | 32252, 32284, | 32636, 32668, | | 33020, 33052, |
| | | 31932, 31964 | 32316, 32348 | 32700, 32732 | | 33084, 33116 |
| 31453 | 31485, | 31613, 31645, | 31997, 32029, | 32381, 32413, | | 32765, 32797, |
| | 31517, | 31677, 31709, | 32061, 32093, | 32445, 32477, | | 32829, 32861, |
| | 31549, | 31741, 31773, | 32125, 32157, | 32509, 32541, | | 32893, 32925, |
| | 31581 | 31805, 31837, | 32189, 32221, | 32573, 32605, | | 32957, 32989, |
| | | 31869, 31901, | 32253, 32285, | 32637, 32669, | | 33021, 33053, |
| | | 31933, 31965 | 32317, 32349 | 32701, 32733 | | 33085, 33117 |
| 31454 | 31486, | 31614, 31646, | 31998, 32030, | 32382, 32414, | | 32766, 32798, |
| | 31518, | 31678, 31710, | 32062, 32094, | 32446, 32478, | | 32830, 32862, |
| | 31550, | 31742, 31774, | 32126, 32158, | 32510, 32542, | | 32894, 32926, |
| | 31582 | 31806, 31838, | 32190, 32222, | 32574, 32606, | | 32958, 32990, |
| | | 31870, 31902, | 32254, 32286, | 32638, 32670, | | 33022, 33054, |
| | | 31934, 31966 | 32318, 32350 | 32702, 32734 | | 33086, 33118 |
| 31455 | 31487, | 31615, 31647, | 31999, 32031, | 32383, 32415, | | 32767, 32799, |
| | 31519, | 31679, 31711, | 32063, 32095, | 32447, 32479, | | 32831, 32863, |
| | 31551, | 31743, 31775, | 32127, 32159, | 32511, 32543, | | 32895, 32927, |
| | 31583 | 31807, 31839, | 32191, 32223, | 32575, 32607, | | 32959, 32991, |
| | | 31871, 31903, | 32255, 32287, | 32639, 32671, | | 33023, 33055, |
| | | 31935, 31967 | 32319, 32351 | 32703, 32735 | | 33087, 33119 |
| 31456 | 31488, | 31616, 31648, | 32000, 32032, | 32384, 32416, | | 32768, 32800, |
| | 31520, | 31680, 31712, | 32064, 32096, | 32448, 32480, | | 32832, 32864, |
| | 31552, | 31744, 31776, | 32128, 32160, | 32512, 32544, | | 32896, 32928, |
| | 31584 | 31808, 31840, | 32192, 32224, | 32576, 32608, | | 32960, 32992, |
| | | 31872, 31904, | 32256, 32288, | 32640, 32672, | | 33024, 33056, |
| | | 31936, 31968 | 32320, 32352 | 32704, 32736 | | 33088, 33120 |
| 31457 | 31489, | 31617, 31649, | 32001, 32033, | 32385, 32417, | | 32769, 32801, |
| | 31521, | 31681, 31713, | 32065, 32097, | 32449, 32481, | | 32833, 32865, |
| | 31553, | 31745, 31777, | 32129, 32161, | 32513, 32545, | | 32897, 32929, |
| | 31585 | 31809, 31841, | 32193, 32225, | 32577, 32609, | | 32961, 32993, |
| | | 31873, 31905, | 32257, 32289, | 32641, 32673, | | 33025, 33057, |
| | | 31937, 31969 | 32321, 32353 | 32705, 32737 | | 33089, 33121 |
| 31458 | 31490, | 31618, 31650, | 32002, 32034, | 32386, 32418, | | 32770, 32802, |
| | 31522, | 31682, 31714, | 32066, 32098, | 32450, 32482, | | 32834, 32866, |
| | 31554, | 31746, 31778, | 32130, 32162, | 32514, 32546, | | 32898, 32930, |
| | 31586 | 31810, 31842, | 32194, 32226, | 32578, 32610, | | 32962, 32994, |
| | | 31874, 31906, | 32258, 32290, | 32642, 32674, | | 33026, 33058, |
| | | 31938, 31970 | 32322, 32354 | 32706, 32738 | | 33090, 33122 |
| 31459 | 31491, | 31619, 31651, | 32003, 32035, | 32387, 32419, | | 32771, 32803, |
| | 31523, | 31683, 31715, | 32067, 32099, | 32451, 32483, | | 32835, 32867, |
| | 31555, | 31747, 31779, | 32131, 32163, | 32515, 32547, | | 32899, 32931, |
| | 31587 | 31811, 31843, | 32195, 32227, | 32579, 32611, | | 32963, 32995, |
| | | 31875, 31907, | 32259, 32291, | 32643, 32675, | | 33027, 33059, |
| | | 31939, 31971 | 32323, 32355 | 32707, 32739 | | 33091, 33123 |
| 31460 | 31492, | 31620, 31652, | 32004, 32036, | 32388, 32420, | | 32772, 32804, |
| | 31524, | 31684, 31716, | 32068, 32100, | 32452, 32484, | | 32836, 32868, |
| | 31556, | 31748, 31780, | 32132, 32164, | 32516, 32548, | | 32900, 32932, |
| | 31588 | 31812, 31844, | 32196, 32228, | 32580, 32612, | | 32964, 32996, |
| | | 31876, 31908, | 32260, 32292, | 32644, 32676, | | 33028, 33060, |
| | | 31940, 31972 | 32324, 32356 | 32708, 32740 | | 33092, 33124 |

TABLE 3.8
Preferred LASV prefusion-stabilized (GPCmut8) constructs

| | | RNA designs and SEQ ID NOs | | | | |
|---|---|---|---|---|---|---|
| PRT | CDS | a-1 | a-2 | b-4 | i-2 | i-3 |
| 33125 | 33157, | 33285, 33317, | 33669, 33701, | 34053, 34085, | | 34437, 34469, |
| | 33189, | 33349, 33381, | 33733, 33765, | 34117, 34149, | | 34501, 34533, |
| | 33221, | 33413, 33445, | 33797, 33829, | 34181, 34213, | | 34565, 34597, |
| | 33253 | 33477, 33509, | 33861, 33893, | 34245, 34277, | | 34629, 34661, |
| | | 33541, 33573, | 33925, 33957, | 34309, 34341, | | 34693, 34725, |
| | | 33605, 33637 | 33989, 34021 | 34373, 34405 | | 34757, 34789 |
| 33126 | 33158, | 33286, 33318, | 33670, 33702, | 34054, 34086, | | 34438, 34470, |
| | 33190, | 33350, 33382, | 33734, 33766, | 34118, 34150, | | 34502, 34534, |
| | 33222, | 33414, 33446, | 33798, 33830, | 34182, 34214, | | 34566, 34598, |
| | 33254 | 33478, 33510, | 33862, 33894, | 34246, 34278, | | 34630, 34662, |
| | | 33542, 33574, | 33926, 33958, | 34310, 34342, | | 34694, 34726, |
| | | 33606, 33638 | 33990, 34022 | 34374, 34406 | | 34758, 34790 |
| 33127 | 33159, | 33287, 33319, | 33671, 33703, | 34055, 34087, | | 34439, 34471, |
| | 33191, | 33351, 33383, | 33735, 33767, | 34119, 34151, | | 34503, 34535, |
| | 33223, | 33415, 33447, | 33799, 33831, | 34183, 34215, | | 34567, 34599, |
| | 33255 | 33479, 33511, | 33863, 33895, | 34247, 34279, | | 34631, 34663, |
| | | 33543, 33575, | 33927, 33959, | 34311, 34343, | | 34695, 34727, |
| | | 33607, 33639 | 33991, 34023 | 34375, 34407 | | 34759, 34791 |
| 33128 | 33160, | 33288, 33320, | 33672, 33704, | 34056, 34088, | | 34440, 34472, |
| | 33192, | 33352, 33384, | 33736, 33768, | 34120, 34152, | | 34504, 34536, |
| | 33224, | 33416, 33448, | 33800, 33832, | 34184, 34216, | | 34568, 34600, |
| | 33256 | 33480, 33512, | 33864, 33896, | 34248, 34280, | | 34632, 34664, |
| | | 33544, 33576, | 33928, 33960, | 34312, 34344, | | 34696, 34728, |
| | | 33608, 33640 | 33992, 34024 | 34376, 34408 | | 34760, 34792 |
| 33129 | 33161, | 33289, 33321, | 33673, 33705, | 34057, 34089, | | 34441, 34473, |
| | 33193, | 33353, 33385, | 33737, 33769, | 34121, 34153, | | 34505, 34537, |
| | 33225, | 33417, 33449, | 33801, 33833, | 34185, 34217, | | 34569, 34601, |
| | 33257 | 33481, 33513, | 33865, 33897, | 34249, 34281, | | 34633, 34665, |
| | | 33545, 33577, | 33929, 33961, | 34313, 34345, | | 34697, 34729, |
| | | 33609, 33641 | 33993, 34025 | 34377, 34409 | | 34761, 34793 |
| 33130 | 33162, | 33290, 33322, | 33674, 33706, | 34058, 34090, | | 34442, 34474, |
| | 33194, | 33354, 33386, | 33738, 33770, | 34122, 34154, | | 34506, 34538, |
| | 33226, | 33418, 33450, | 33802, 33834, | 34186, 34218, | | 34570, 34602, |
| | 33258 | 33482, 33514, | 33866, 33898, | 34250, 34282, | | 34634, 34666, |
| | | 33546, 33578, | 33930, 33962, | 34314, 34346, | | 34698, 34730, |
| | | 33610, 33642 | 33994, 34026 | 34378, 34410 | | 34762, 34794 |
| 33131 | 33163, | 33291, 33323, | 33675, 33707, | 34059, 34091, | | 34443, 34475, |
| | 33195, | 33355, 33387, | 33739, 33771, | 34123, 34155, | | 34507, 34539, |
| | 33227, | 33419, 33451, | 33803, 33835, | 34187, 34219, | | 34571, 34603, |
| | 33259 | 33483, 33515, | 33867, 33899, | 34251, 34283, | | 34635, 34667, |
| | | 33547, 33579, | 33931, 33963, | 34315, 34347, | | 34699, 34731, |
| | | 33611, 33643 | 33995, 34027 | 34379, 34411 | | 34763, 34795 |
| 33132 | 33164, | 33292, 33324, | 33676, 33708, | 34060, 34092, | | 34444, 34476, |
| | 33196, | 33356, 33388, | 33740, 33772, | 34124, 34156, | | 34508, 34540, |
| | 33228, | 33420, 33452, | 33804, 33836, | 34188, 34220, | | 34572, 34604, |
| | 33260 | 33484, 33516, | 33868, 33900, | 34252, 34284, | | 34636, 34668, |
| | | 33548, 33580, | 33932, 33964, | 34316, 34348, | | 34700, 34732, |
| | | 33612, 33644 | 33996, 34028 | 34380, 34412 | | 34764, 34796 |
| 33133 | 33165, | 33293, 33325, | 33677, 33709, | 34061, 34093, | | 34445, 34477, |
| | 33197, | 33357, 33389, | 33741, 33773, | 34125, 34157, | | 34509, 34541, |
| | 33229, | 33421, 33453, | 33805, 33837, | 34189, 34221, | | 34573, 34605, |
| | 33261 | 33485, 33517, | 33869, 33901, | 34253, 34285, | | 34637, 34669, |
| | | 33549, 33581, | 33933, 33965, | 34317, 34349, | | 34701, 34733, |
| | | 33613, 33645 | 33997, 34029 | 34381, 34413 | | 34765, 34797 |
| 33134 | 33166, | 33294, 33326, | 33678, 33710, | 34062, 34094, | | 34446, 34478, |
| | 33198, | 33358, 33390, | 33742, 33774, | 34126, 34158, | | 34510, 34542, |
| | 33230, | 33422, 33454, | 33806, 33838, | 34190, 34222, | | 34574, 34606, |
| | 33262 | 33486, 33518, | 33870, 33902, | 34254, 34286, | | 34638, 34670, |
| | | 33550, 33582, | 33934, 33966, | 34318, 34350, | | 34702, 34734, |
| | | 33614, 33646 | 33998, 34030 | 34382, 34414 | | 34766, 34798 |
| 33135 | 33167, | 33295, 33327, | 33679, 33711, | 34063, 34095, | | 34447, 34479, |
| | 33199, | 33359, 33391, | 33743, 33775, | 34127, 34159, | | 34511, 34543, |
| | 33231, | 33423, 33455, | 33807, 33839, | 34191, 34223, | | 34575, 34607, |
| | 33263 | 33487, 33519, | 33871, 33903, | 34255, 34287, | | 34639, 34671, |
| | | 33551, 33583, | 33935, 33967, | 34319, 34351, | | 34703, 34735, |
| | | 33615, 33647 | 33999, 34031 | 34383, 34415 | | 34767, 34799 |
| 33136 | 33168, | 33296, 33328, | 33680, 33712, | 34064, 34096, | | 34448, 34480, |
| | 33200, | 33360, 33392, | 33744, 33776, | 34128, 34160, | | 34512, 34544, |
| | 33232, | 33424, 33456, | 33808, 33840, | 34192, 34224, | | 34576, 34608, |
| | 33264 | 33488, 33520, | 33872, 33904, | 34256, 34288, | | 34640, 34672, |
| | | 33552, 33584, | 33936, 33968, | 34320, 34352, | | 34704, 34736, |
| | | 33616, 33648 | 34000, 34032 | 34384, 34416 | | 34768, 34800 |
| 33137 | 33169, | 33297, 33329, | 33681, 33713, | 34065, 34097, | | 34449, 34481, |
| | 33201, | 33361, 33393, | 33745, 33777, | 34129, 34161, | | 34513, 34545, |
| | 33233, | 33425, 33457, | 33809, 33841, | 34193, 34225, | | 34577, 34609, |
| | 33265 | 33489, 33521, | 33873, 33905, | 34257, 34289, | | 34641, 34673, |
| | | 33553, 33585, | 33937, 33969, | 34321, 34353, | | 34705, 34737, |
| | | 33617, 33649 | 34001, 34033 | 34385, 34417 | | 34769, 34801 |
| 33138 | 33170, | 33298, 33330, | 33682, 33714, | 34066, 34098, | | 34450, 34482, |
| | 33202, | 33362, 33394, | 33746, 33778, | 34130, 34162, | | 34514, 34546, |
| | 33234, | 33426, 33458, | 33810, 33842, | 34194, 34226, | | 34578, 34610, |
| | 33266 | 33490, 33522, | 33874, 33906, | 34258, 34290, | | 34642, 34674, |
| | | 33554, 33586, | 33938, 33970, | 34322, 34354, | | 34706, 34738, |
| | | 33618, 33650 | 34002, 34034 | 34386, 34418 | | 34770, 34802 |
| 33139 | 33171, | 33299, 33331, | 33683, 33715, | 34067, 34099, | | 34451, 34483, |
| | 33203, | 33363, 33395, | 33747, 33779, | 34131, 34163, | | 34515, 34547, |
| | 33235, | 33427, 33459, | 33811, 33843, | 34195, 34227, | | 34579, 34611, |
| | 33267 | 33491, 33523, | 33875, 33907, | 34259, 34291, | | 34643, 34675, |
| | | 33555, 33587, | 33939, 33971, | 34323, 34355, | | 34707, 34739, |
| | | 33619, 33651 | 34003, 34035 | 34387, 34419 | | 34771, 34803 |
| 33140 | 33172, | 33300, 33332, | 33684, 33716, | 34068, 34100, | | 34452, 34484, |
| | 33204, | 33364, 33396, | 33748, 33780, | 34132, 34164, | | 34516, 34548, |
| | 33236, | 33428, 33460, | 33812, 33844, | 34196, 34228, | | 34580, 34612, |
| | 33268 | 33492, 33524, | 33876, 33908, | 34260, 34292, | | 34644, 34676, |
| | | 33556, 33588, | 33940, 33972, | 34324, 34356, | | 34708, 34740, |
| | | 33620, 33652 | 34004, 34036 | 34388, 34420 | | 34772, 34804 |
| 33141 | 33173, | 33301, 33333, | 33685, 33717, | 34069, 34101, | | 34453, 34485, |
| | 33205, | 33365, 33397, | 33749, 33781, | 34133, 34165, | | 34517, 34549, |
| | 33237, | 33429, 33461, | 33813, 33845, | 34197, 34229, | | 34581, 34613, |
| | 33269 | 33493, 33525, | 33877, 33909, | 34261, 34293, | | 34645, 34677, |
| | | 33557, 33589, | 33941, 33973, | 34325, 34357, | | 34709, 34741, |
| | | 33621, 33653 | 34005, 34037 | 34389, 34421 | | 34773, 34805 |
| 33142 | 33174, | 33302, 33334, | 33686, 33718, | 34070, 34102, | | 34454, 34486, |
| | 33206, | 33366, 33398, | 33750, 33782, | 34134, 34166, | | 34518, 34550, |
| | 33238, | 33430, 33462, | 33814, 33846, | 34198, 34230, | | 34582, 34614, |
| | 33270 | 33494, 33526, | 33878, 33910, | 34262, 34294, | | 34646, 34678, |
| | | 33558, 33590, | 33942, 33974, | 34326, 34358, | | 34710, 34742, |
| | | 33622, 33654 | 34006, 34038 | 34390, 34422 | | 34774, 34806 |
| 33143 | 33175, | 33303, 33335, | 33687, 33719, | 34071, 34103, | | 34455, 34487, |
| | 33207, | 33367, 33399, | 33751, 33783, | 34135, 34167, | | 34519, 34551, |
| | 33239, | 33431, 33463, | 33815, 33847, | 34199, 34231, | | 34583, 34615, |
| | 33271 | 33495, 33527, | 33879, 33911, | 34263, 34295, | | 34647, 34679, |
| | | 33559, 33591, | 33943, 33975, | 34327, 34359, | | 34711, 34743, |
| | | 33623, 33655 | 34007, 34039 | 34391, 34423 | | 34775, 34807 |
| 33144 | 33176, | 33304, 33336, | 33688, 33720, | 34072, 34104, | | 34456, 34488, |
| | 33208, | 33368, 33400, | 33752, 33784, | 34136, 34168, | | 34520, 34552, |
| | 33240, | 33432, 33464, | 33816, 33848, | 34200, 34232, | | 34584, 34616, |
| | 33272 | 33496, 33528, | 33880, 33912, | 34264, 34296, | | 34648, 34680, |
| | | 33560, 33592, | 33944, 33976, | 34328, 34360, | | 34712, 34744, |
| | | 33624, 33656 | 34008, 34040 | 34392, 34424 | | 34776, 34808 |
| 33145 | 33177, | 33305, 33337, | 33689, 33721, | 34073, 34105, | | 34457, 34489, |
| | 33209, | 33369, 33401, | 33753, 33785, | 34137, 34169, | | 34521, 34553, |
| | 33241, | 33433, 33465, | 33817, 33849, | 34201, 34233, | | 34585, 34617, |
| | 33273 | 33497, 33529, | 33881, 33913, | 34265, 34297, | | 34649, 34681, |
| | | 33561, 33593, | 33945, 33977, | 34329, 34361, | | 34713, 34745, |
| | | 33625, 33657 | 34009, 34041 | 34393, 34425 | | 34777, 34809 |
| 33146 | 33178, | 33306, 33338, | 33690, 33722, | 34074, 34106, | | 34458, 34490, |
| | 33210, | 33370, 33402, | 33754, 33786, | 34138, 34170, | | 34522, 34554, |
| | 33242, | 33434, 33466, | 33818, 33850, | 34202, 34234, | | 34586, 34618, |
| | 33274 | 33498, 33530, | 33882, 33914, | 34266, 34298, | | 34650, 34682, |
| | | 33562, 33594, | 33946, 33978, | 34330, 34362, | | 34714, 34746, |
| | | 33626, 33658 | 34010, 34042 | 34394, 34426 | | 34778, 34810 |
| 33147 | 33179, | 33307, 33339, | 33691, 33723, | 34075, 34107, | | 34459, 34491, |
| | 33211, | 33371, 33403, | 33755, 33787, | 34139, 34171, | | 34523, 34555, |
| | 33243, | 33435, 33467, | 33819, 33851, | 34203, 34235, | | 34587, 34619, |
| | 33275 | 33499, 33531, | 33883, 33915, | 34267, 34299, | | 34651, 34683, |
| | | 33563, 33595, | 33947, 33979, | 34331, 34363, | | 34715, 34747, |
| | | 33627, 33659 | 34011, 34043 | 34395, 34427 | | 34779, 34811 |
| 33148 | 33180, | 33308, 33340, | 33692, 33724, | 34076, 34108, | | 34460, 34492, |
| | 33212, | 33372, 33404, | 33756, 33788, | 34140, 34172, | | 34524, 34556, |
| | 33244, | 33436, 33468, | 33820, 33852, | 34204, 34236, | | 34588, 34620, |
| | 33276 | 33500, 33532, | 33884, 33916, | 34268, 34300, | | 34652, 34684, |
| | | 33564, 33596, | 33948, 33980, | 34332, 34364, | | 34716, 34748, |
| | | 33628, 33660 | 34012, 34044 | 34396, 34428 | | 34780, 34812 |
| 33149 | 33181, | 33309, 33341, | 33693, 33725, | 34077, 34109, | | 34461, 34493, |
| | 33213, | 33373, 33405, | 33757, 33789, | 34141, 34173, | | 34525, 34557, |
| | 33245, | 33437, 33469, | 33821, 33853, | 34205, 34237, | | 34589, 34621, |
| | 33277 | 33501, 33533, | 33885, 33917, | 34269, 34301, | | 34653, 34685, |
| | | 33565, 33597, | 33949, 33981, | 34333, 34365, | | 34717, 34749, |
| | | 33629, 33661 | 34013, 34045 | 34397, 34429 | | 34781, 34813 |
| 33150 | 33182, | 33310, 33342, | 33694, 33726, | 34078, 34110, | | 34462, 34494, |
| | 33214, | 33374, 33406, | 33758, 33790, | 34142, 34174, | | 34526, 34558, |
| | 33246, | 33438, 33470, | 33822, 33854, | 34206, 34238, | | 34590, 34622, |
| | 33278 | 33502, 33534, | 33886, 33918, | 34270, 34302, | | 34654, 34686, |
| | | 33566, 33598, | 33950, 33982, | 34334, 34366, | | 34718, 34750, |
| | | 33630, 33662 | 34014, 34046 | 34398, 34430 | | 34782, 34814 |
| 33151 | 33183, | 33311, 33343, | 33695, 33727, | 34079, 34111, | | 34463, 34495, |
| | 33215, | 33375, 33407, | 33759, 33791, | 34143, 34175, | | 34527, 34559, |
| | 33247, | 33439, 33471, | 33823, 33855, | 34207, 34239, | | 34591, 34623, |
| | 33279 | 33503, 33535, | 33887, 33919, | 34271, 34303, | | 34655, 34687, |
| | | 33567, 33599, | 33951, 33983, | 34335, 34367, | | 34719, 34751, |
| | | 33631, 33663 | 34015, 34047 | 34399, 34431 | | 34783, 34815 |
| 33152 | 33184, | 33312, 33344, | 33696, 33728, | 34080, 34112, | | 34464, 34496, |
| | 33216, | 33376, 33408, | 33760, 33792, | 34144, 34176, | | 34528, 34560, |
| | 33248, | 33440, 33472, | 33824, 33856, | 34208, 34240, | | 34592, 34624, |
| | 33280 | 33504, 33536, | 33888, 33920, | 34272, 34304, | | 34656, 34688, |
| | | 33568, 33600, | 33952, 33984, | 34336, 34368, | | 34720, 34752, |
| | | 33632, 33664 | 34016, 34048 | 34400, 34432 | | 34784, 34816 |
| 33153 | 33185, | 33313, 33345, | 33697, 33729, | 34081, 34113, | | 34465, 34497, |
| | 33217, | 33377, 33409, | 33761, 33793, | 34145, 34177, | | 34529, 34561, |
| | 33249, | 33441, 33473, | 33825, 33857, | 34209, 34241, | | 34593, 34625, |
| | 33281 | 33505, 33537, | 33889, 33921, | 34273, 34305, | | 34657, 34689, |
| | | 33569, 33601, | 33953, 33985, | 34337, 34369, | | 34721, 34753, |
| | | 33633, 33665 | 34017, 34049 | 34401, 34433 | | 34785, 34817 |
| 33154 | 33186, | 33314, 33346, | 33698, 33730, | 34082, 34114, | | 34466, 34498, |
| | 33218, | 33378, 33410, | 33762, 33794, | 34146, 34178, | | 34530, 34562, |
| | 33250, | 33442, 33474, | 33826, 33858, | 34210, 34242, | | 34594, 34626, |
| | 33282 | 33506, 33538, | 33890, 33922, | 34274, 34306, | | 34658, 34690, |
| | | 33570, 33602, | 33954, 33986, | 34338, 34370, | | 34722, 34754, |
| | | 33634, 33666 | 34018, 34050 | 34402, 34434 | | 34786, 34818 |
| 33155 | 33187, | 33315, 33347, | 33699, 33731, | 34083, 34115, | | 34467, 34499, |
| | 33219, | 33379, 33411, | 33763, 33795, | 34147, 34179, | | 34531, 34563, |
| | 33251, | 33443, 33475, | 33827, 33859, | 34211, 34243, | | 34595, 34627, |
| | 33283 | 33507, 33539, | 33891, 33923, | 34275, 34307, | | 34659, 34691, |
| | | 33571, 33603, | 33955, 33987, | 34339, 34371, | | 34723, 34755, |
| | | 33635, 33667 | 34019, 34051 | 34403, 34435 | | 34787, 34819 |
| 33156 | 33188, | 33316, 33348, | 33700, 33732, | 34084, 34116, | | 34468, 34500, |
| | 33220, | 33380, 33412, | 33764, 33796, | 34148, 34180, | | 34532, 34564, |
| | 33252, | 33444, 33476, | 33828, 33860, | 34212, 34244, | | 34596, 34628, |
| | 33284 | 33508, 33540, | 33892, 33924, | 34276, 34308, | | 34660, 34692, |
| | | 33572, 33604, | 33956, 33988, | 34340, 34372, | | 34724, 34756, |
| | | 33636, 33668 | 34020, 34052 | 34404, 34436 | | 34788, 34820 |

TABLE 3.9
Preferred LASV prefusion-stabilized (GPCmut9) constructs

| | | RNA designs and SEQ ID NOs | | | | |
|---|---|---|---|---|---|---|
| PRT | CDS | a-1 | a-2 | b-4 | i-2 | i-3 |
| 34821 | 34853, | 34981, 35013, | 35365, 35397, | 35749, 35781, | | 36133, 36165, |
| | 34885, | 35045, 35077, | 35429, 35461, | 35813, 35845, | | 36197, 36229, |
| | 34917, | 35109, 35141, | 35493, 35525, | 35877, 35909, | | 36261, 36293, |
| | 34949 | 35173, 35205, | 35557, 35589, | 35941, 35973, | | 36325, 36357, |
| | | 35237, 35269, | 35621, 35653, | 36005, 36037, | | 36389, 36421, |
| | | 35301, 35333 | 35685, 35717 | 36069, 36101 | | 36453, 36485 |
| 34822 | 34854, | 34982, 35014, | 35366, 35398, | 35750, 35782, | | 36134, 36166, |
| | 34886, | 35046, 35078, | 35430, 35462, | 35814, 35846, | | 36198, 36230, |
| | 34918, | 35110, 35142, | 35494, 35526, | 35878, 35910, | | 36262, 36294, |
| | 34950 | 35174, 35206, | 35558, 35590, | 35942, 35974, | | 36326, 36358, |
| | | 35238, 35270, | 35622, 35654, | 36006, 36038, | | 36390, 36422, |
| | | 35302, 35334 | 35686, 35718 | 36070, 36102 | | 36454, 36486 |
| 34823 | 34855, | 34983, 35015, | 35367, 35399, | 35751, 35783, | | 36135, 36167, |
| | 34887, | 35047, 35079, | 35431, 35463, | 35815, 35847, | | 36199, 36231, |
| | 34919, | 35111, 35143, | 35495, 35527, | 35879, 35911, | | 36263, 36295, |
| | 34951 | 35175, 35207, | 35559, 35591, | 35943, 35975, | | 36327, 36359, |
| | | 35239, 35271, | 35623, 35655, | 36007, 36039, | | 36391, 36423, |
| | | 35303, 35335 | 35687, 35719 | 36071, 36103 | | 36455, 36487 |
| 34824 | 34856, | 34984, 35016, | 35368, 35400, | 35752, 35784, | | 36136, 36168, |
| | 34888, | 35048, 35080, | 35432, 35464, | 35816, 35848, | | 36200, 36232, |
| | 34920, | 35112, 35144, | 35496, 35528, | 35880, 35912, | | 36264, 36296, |
| | 34952 | 35176, 35208, | 35560, 35592, | 35944, 35976, | | 36328, 36360, |
| | | 35240, 35272, | 35624, 35656, | 36008, 36040, | | 36392, 36424, |
| | | 35304, 35336 | 35688, 35720 | 36072, 36104 | | 36456, 36488 |
| 34825 | 34857, | 34985, 35017, | 35369, 35401, | 35753, 35785, | | 36137, 36169, |
| | 34889, | 35049, 35081, | 35433, 35465, | 35817, 35849, | | 36201, 36233, |
| | 34921, | 35113, 35145, | 35497, 35529, | 35881, 35913, | | 36265, 36297, |
| | 34953 | 35177, 35209, | 35561, 35593, | 35945, 35977, | | 36329, 36361, |
| | | 35241, 35273, | 35625, 35657, | 36009, 36041, | | 36393, 36425, |
| | | 35305, 35337 | 35689, 35721 | 36073, 36105 | | 36457, 36489 |
| 34826 | 34858, | 34986, 35018, | 35370, 35402, | 35754, 35786, | | 36138, 36170, |
| | 34890, | 35050, 35082, | 35434, 35466, | 35818, 35850, | | 36202, 36234, |
| | 34922, | 35114, 35146, | 35498, 35530, | 35882, 35914, | | 36266, 36298, |
| | 34954 | 35178, 35210, | 35562, 35594, | 35946, 35978, | | 36330, 36362, |
| | | 35242, 35274, | 35626, 35658, | 36010, 36042, | | 36394, 36426, |
| | | 35306, 35338 | 35690, 35722 | 36074, 36106 | | 36458, 36490 |
| 34827 | 34859, | 34987, 35019, | 35371, 35403, | 35755, 35787, | | 36139, 36171, |
| | 34891, | 35051, 35083, | 35435, 35467, | 35819, 35851, | | 36203, 36235, |
| | 34923, | 35115, 35147, | 35499, 35531, | 35883, 35915, | | 36267, 36299, |
| | 34955 | 35179, 35211, | 35563, 35595, | 35947, 35979, | | 36331, 36363, |
| | | 35243, 35275, | 35627, 35659, | 36011, 36043, | | 36395, 36427, |
| | | 35307, 35339 | 35691, 35723 | 36075, 36107 | | 36459, 36491 |
| 34828 | 34860, | 34988, 35020, | 35372, 35404, | 35756, 35788, | | 36140, 36172, |
| | 34892, | 35052, 35084, | 35436, 35468, | 35820, 35852, | | 36204, 36236, |
| | 34924, | 35116, 35148, | 35500, 35532, | 35884, 35916, | | 36268, 36300, |
| | 34956 | 35180, 35212, | 35564, 35596, | 35948, 35980, | | 36332, 36364, |
| | | 35244, 35276, | 35628, 35660, | 36012, 36044, | | 36396, 36428, |
| | | 35308, 35340 | 35692, 35724 | 36076, 36108 | | 36460, 36492 |
| 34829 | 34861, | 34989, 35021, | 35373, 35405, | 35757, 35789, | | 36141, 36173, |
| | 34893, | 35053, 35085, | 35437, 35469, | 35821, 35853, | | 36205, 36237, |
| | 34925, | 35117, 35149, | 35501, 35533, | 35885, 35917, | | 36269, 36301, |
| | 34957 | 35181, 35213, | 35565, 35597, | 35949, 35981, | | 36333, 36365, |
| | | 35245, 35277, | 35629, 35661, | 36013, 36045, | | 36397, 36429, |
| | | 35309, 35341 | 35693, 35725 | 36077, 36109 | | 36461, 36493 |
| 34830 | 34862, | 34990, 35022, | 35374, 35406, | 35758, 35790, | | 36142, 36174, |
| | 34894, | 35054, 35086, | 35438, 35470, | 35822, 35854, | | 36206, 36238, |
| | 34926, | 35118, 35150, | 35502, 35534, | 35886, 35918, | | 36270, 36302, |
| | 34958 | 35182, 35214, | 35566, 35598, | 35950, 35982, | | 36334, 36366, |
| | | 35246, 35278, | 35630, 35662, | 36014, 36046, | | 36398, 36430, |
| | | 35310, 35342 | 35694, 35726 | 36078, 36110 | | 36462, 36494 |
| 34831 | 34863, | 34991, 35023, | 35375, 35407, | 35759, 35791, | | 36143, 36175, |
| | 34895, | 35055, 35087, | 35439, 35471, | 35823, 35855, | | 36207, 36239, |
| | 34927, | 35119, 35151, | 35503, 35535, | 35887, 35919, | | 36271, 36303, |
| | 34959 | 35183, 35215, | 35567, 35599, | 35951, 35983, | | 36335, 36367, |
| | | 35247, 35279, | 35631, 35663, | 36015, 36047, | | 36399, 36431, |
| | | 35311, 35343 | 35695, 35727 | 36079, 36111 | | 36463, 36495 |
| 34832 | 34864, | 34992, 35024, | 35376, 35408, | 35760, 35792, | | 36144, 36176, |
| | 34896, | 35056, 35088, | 35440, 35472, | 35824, 35856, | | 36208, 36240, |
| | 34928, | 35120, 35152, | 35504, 35536, | 35888, 35920, | | 36272, 36304, |
| | 34960 | 35184, 35216, | 35568, 35600, | 35952, 35984, | | 36336, 36368, |
| | | 35248, 35280, | 35632, 35664, | 36016, 36048, | | 36400, 36432, |
| | | 35312, 35344 | 35696, 35728 | 36080, 36112 | | 36464, 36496 |
| 34833 | 34865, | 34993, 35025, | 35377, 35409, | 35761, 35793, | | 36145, 36177, |
| | 34897, | 35057, 35089, | 35441, 35473, | 35825, 35857, | | 36209, 36241, |
| | 34929, | 35121, 35153, | 35505, 35537, | 35889, 35921, | | 36273, 36305, |
| | 34961 | 35185, 35217, | 35569, 35601, | 35953, 35985, | | 36337, 36369, |
| | | 35249, 35281, | 35633, 35665, | 36017, 36049, | | 36401, 36433, |
| | | 35313, 35345 | 35697, 35729 | 36081, 36113 | | 36465, 36497 |
| 34834 | 34866, | 34994, 35026, | 35378, 35410, | 35762, 35794, | | 36146, 36178, |
| | 34898, | 35058, 35090, | 35442, 35474, | 35826, 35858, | | 36210, 36242, |
| | 34930, | 35122, 35154, | 35506, 35538, | 35890, 35922, | | 36274, 36306, |
| | 34962 | 35186, 35218, | 35570, 35602, | 35954, 35986, | | 36338, 36370, |
| | | 35250, 35282, | 35634, 35666, | 36018, 36050, | | 36402, 36434, |
| | | 35314, 35346 | 35698, 35730 | 36082, 36114 | | 36466, 36498 |
| 34835 | 34867, | 34995, 35027, | 35379, 35411, | 35763, 35795, | | 36147, 36179, |
| | 34899, | 35059, 35091, | 35443, 35475, | 35827, 35859, | | 36211, 36243, |
| | 34931, | 35123, 35155, | 35507, 35539, | 35891, 35923, | | 36275, 36307, |
| | 34963 | 35187, 35219, | 35571, 35603, | 35955, 35987, | | 36339, 36371, |
| | | 35251, 35283, | 35635, 35667, | 36019, 36051, | | 36403, 36435, |
| | | 35315, 35347 | 35699, 35731 | 36083, 36115 | | 36467, 36499 |
| 34836 | 34868, | 34996, 35028, | 35380, 35412, | 35764, 35796, | | 36148, 36180, |
| | 34900, | 35060, 35092, | 35444, 35476, | 35828, 35860, | | 36212, 36244, |
| | 34932, | 35124, 35156, | 35508, 35540, | 35892, 35924, | | 36276, 36308, |
| | 34964 | 35188, 35220, | 35572, 35604, | 35956, 35988, | | 36340, 36372, |
| | | 35252, 35284, | 35636, 35668, | 36020, 36052, | | 36404, 36436, |
| | | 35316, 35348 | 35700, 35732 | 36084, 36116 | | 36468, 36500 |
| 34837 | 34869, | 34997, 35029, | 35381, 35413, | 35765, 35797, | | 36149, 36181, |
| | 34901, | 35061, 35093, | 35445, 35477, | 35829, 35861, | | 36213, 36245, |
| | 34933, | 35125, 35157, | 35509, 35541, | 35893, 35925, | | 36277, 36309, |
| | 34965 | 35189, 35221, | 35573, 35605, | 35957, 35989, | | 36341, 36373, |
| | | 35253, 35285, | 35637, 35669, | 36021, 36053, | | 36405, 36437, |
| | | 35317, 35349 | 35701, 35733 | 36085, 36117 | | 36469, 36501 |
| 34838 | 34870, | 34998, 35030, | 35382, 35414, | 35766, 35798, | | 36150, 36182, |
| | 34902, | 35062, 35094, | 35446, 35478, | 35830, 35862, | | 36214, 36246, |
| | 34934, | 35126, 35158, | 35510, 35542, | 35894, 35926, | | 36278, 36310, |
| | 34966 | 35190, 35222, | 35574, 35606, | 35958, 35990, | | 36342, 36374, |
| | | 35254, 35286, | 35638, 35670, | 36022, 36054, | | 36406, 36438, |
| | | 35318, 35350 | 35702, 35734 | 36086, 36118 | | 36470, 36502 |
| 34839 | 34871, | 34999, 35031, | 35383, 35415, | 35767, 35799, | | 36151, 36183, |
| | 34903, | 35063, 35095, | 35447, 35479, | 35831, 35863, | | 36215, 36247, |
| | 34935, | 35127, 35159, | 35511, 35543, | 35895, 35927, | | 36279, 36311, |
| | 34967 | 35191, 35223, | 35575, 35607, | 35959, 35991, | | 36343, 36375, |
| | | 35255, 35287, | 35639, 35671, | 36023, 36055, | | 36407, 36439, |
| | | 35319, 35351 | 35703, 35735 | 36087, 36119 | | 36471, 36503 |
| 34840 | 34872, | 35000, 35032, | 35384, 35416, | 35768, 35800, | | 36152, 36184, |
| | 34904, | 35064, 35096, | 35448, 35480, | 35832, 35864, | | 36216, 36248, |
| | 34936, | 35128, 35160, | 35512, 35544, | 35896, 35928, | | 36280, 36312, |
| | 34968 | 35192, 35224, | 35576, 35608, | 35960, 35992, | | 36344, 36376, |
| | | 35256, 35288, | 35640, 35672, | 36024, 36056, | | 36408, 36440, |
| | | 35320, 35352 | 35704, 35736 | 36088, 36120 | | 36472, 36504 |
| 34841 | 34873, | 35001, 35033, | 35385, 35417, | 35769, 35801, | | 36153, 36185, |
| | 34905, | 35065, 35097, | 35449, 35481, | 35833, 35865, | | 36217, 36249, |
| | 34937, | 35129, 35161, | 35513, 35545, | 35897, 35929, | | 36281, 36313, |
| | 34969 | 35193, 35225, | 35577, 35609, | 35961, 35993, | | 36345, 36377, |
| | | 35257, 35289, | 35641, 35673, | 36025, 36057, | | 36409, 36441, |
| | | 35321, 35353 | 35705, 35737 | 36089, 36121 | | 36473, 36505 |
| 34842 | 34874, | 35002, 35034, | 35386, 35418, | 35770, 35802, | | 36154, 36186, |
| | 34906, | 35066, 35098, | 35450, 35482, | 35834, 35866, | | 36218, 36250, |
| | 34938, | 35130, 35162, | 35514, 35546, | 35898, 35930, | | 36282, 36314, |
| | 34970 | 35194, 35226, | 35578, 35610, | 35962, 35994, | | 36346, 36378, |
| | | 35258, 35290, | 35642, 35674, | 36026, 36058, | | 36410, 36442, |
| | | 35322, 35354 | 35706, 35738 | 36090, 36122 | | 36474, 36506 |
| 34843 | 34875, | 35003, 35035, | 35387, 35419, | 35771, 35803, | | 36155, 36187, |
| | 34907, | 35067, 35099, | 35451, 35483, | 35835, 35867, | | 36219, 36251, |
| | 34939, | 35131, 35163, | 35515, 35547, | 35899, 35931, | | 36283, 36315, |
| | 34971 | 35195, 35227, | 35579, 35611, | 35963, 35995, | | 36347, 36379, |
| | | 35259, 35291, | 35643, 35675, | 36027, 36059, | | 36411, 36443, |
| | | 35323, 35355 | 35707, 35739 | 36091, 36123 | | 36475, 36507 |
| 34844 | 34876, | 35004, 35036, | 35388, 35420, | 35772, 35804, | | 36156, 36188, |
| | 34908, | 35068, 35100, | 35452, 35484, | 35836, 35868, | | 36220, 36252, |
| | 34940, | 35132, 35164, | 35516, 35548, | 35900, 35932, | | 36284, 36316, |
| | 34972 | 35196, 35228, | 35580, 35612, | 35964, 35996, | | 36348, 36380, |
| | | 35260, 35292, | 35644, 35676, | 36028, 36060, | | 36412, 36444, |
| | | 35324, 35356 | 35708, 35740 | 36092, 36124 | | 36476, 36508 |
| 34845 | 34877, | 35005, 35037, | 35389, 35421, | 35773, 35805, | | 36157, 36189, |
| | 34909, | 35069, 35101, | 35453, 35485, | 35837, 35869, | | 36221, 36253, |
| | 34941, | 35133, 35165, | 35517, 35549, | 35901, 35933, | | 36285, 36317, |
| | 34973 | 35197, 35229, | 35581, 35613, | 35965, 35997, | | 36349, 36381, |
| | | 35261, 35293, | 35645, 35677, | 36029, 36061, | | 36413, 36445, |
| | | 35325, 35357 | 35709, 35741 | 36093, 36125 | | 36477, 36509 |
| 34846 | 34878, | 35006, 35038, | 35390, 35422, | 35774, 35806, | | 36158, 36190, |
| | 34910, | 35070, 35102, | 35454, 35486, | 35838, 35870, | | 36222, 36254, |
| | 34942, | 35134, 35166, | 35518, 35550, | 35902, 35934, | | 36286, 36318, |
| | 34974 | 35198, 35230, | 35582, 35614, | 35966, 35998, | | 36350, 36382, |
| | | 35262, 35294, | 35646, 35678, | 36030, 36062, | | 36414, 36446, |
| | | 35326, 35358 | 35710, 35742 | 36094, 36126 | | 36478, 36510 |
| 34847 | 34879, | 35007, 35039, | 35391, 35423, | 35775, 35807, | | 36159, 36191, |
| | 34911, | 35071, 35103, | 35455, 35487, | 35839, 35871, | | 36223, 36255, |
| | 34943, | 35135, 35167, | 35519, 35551, | 35903, 35935, | | 36287, 36319, |
| | 34975 | 35199, 35231, | 35583, 35615, | 35967, 35999, | | 36351, 36383, |
| | | 35263, 35295, | 35647, 35679, | 36031, 36063, | | 36415, 36447, |
| | | 35327, 35359 | 35711, 35743 | 36095, 36127 | | 36479, 36511 |
| 34848 | 34880, | 35008, 35040, | 35392, 35424, | 35776, 35808, | | 36160, 36192, |
| | 34912, | 35072, 35104, | 35456, 35488, | 35840, 35872, | | 36224, 36256, |
| | 34944, | 35136, 35168, | 35520, 35552, | 35904, 35936, | | 36288, 36320, |
| | 34976 | 35200, 35232, | 35584, 35616, | 35968, 36000, | | 36352, 36384, |
| | | 35264, 35296, | 35648, 35680, | 36032, 36064, | | 36416, 36448, |
| | | 35328, 35360 | 35712, 35744 | 36096, 36128 | | 36480, 36512 |
| 34849 | 34881, | 35009, 35041, | 35393, 35425, | 35777, 35809, | | 36161, 36193, |
| | 34913, | 35073, 35105, | 35457, 35489, | 35841, 35873, | | 36225, 36257, |
| | 34945, | 35137, 35169, | 35521, 35553, | 35905, 35937, | | 36289, 36321, |
| | 34977 | 35201, 35233, | 35585, 35617, | 35969, 36001, | | 36353, 36385, |
| | | 35265, 35297, | 35649, 35681, | 36033, 36065, | | 36417, 36449, |
| | | 35329, 35361 | 35713, 35745 | 36097, 36129 | | 36481, 36513 |
| 34850 | 34882, | 35010, 35042, | 35394, 35426, | 35778, 35810, | | 36162, 36194, |
| | 34914, | 35074, 35106, | 35458, 35490, | 35842, 35874, | | 36226, 36258, |
| | 34946, | 35138, 35170, | 35522, 35554, | 35906, 35938, | | 36290, 36322, |
| | 34978 | 35202, 35234, | 35586, 35618, | 35970, 36002, | | 36354, 36386, |
| | | 35266, 35298, | 35650, 35682, | 36034, 36066, | | 36418, 36450, |
| | | 35330, 35362 | 35714, 35746 | 36098, 36130 | | 36482, 36514 |
| 34851 | 34883, | 35011, 35043, | 35395, 35427, | 35779, 35811, | | 36163, 36195, |
| | 34915, | 35075, 35107, | 35459, 35491, | 35843, 35875, | | 36227, 36259, |
| | 34947, | 35139, 35171, | 35523, 35555, | 35907, 35939, | | 36291, 36323, |
| | 34979 | 35203, 35235, | 35587, 35619, | 35971, 36003, | | 36355, 36387, |
| | | 35267, 35299, | 35651, 35683, | 36035, 36067, | | 36419, 36451, |
| | | 35331, 35363 | 35715, 35747 | 36099, 36131 | | 36483, 36515 |
| 34852 | 34884, | 35012, 35044, | 35396, 35428, | 35780, 35812, | | 36164, 36196, |
| | 34916, | 35076, 35108, | 35460, 35492, | 35844, 35876, | | 36228, 36260, |
| | 34948, | 35140, 35172, | 35524, 35556, | 35908, 35940, | | 36292, 36324, |
| | 34980 | 35204, 35236, | 35588, 35620, | 35972, 36004, | | 36356, 36388, |
| | | 35268, 35300, | 35652, 35684, | 36036, 36068, | | 36420, 36452, |
| | | 35332, 35364 | 35716, 35748 | 36100, 36132 | | 36484, 36516 |

TABLE 3.10
Preferred LASV prefusion-stabilized (GPCmut10) constructs

| | | RNA designs and SEQ ID NOs | | | | |
|---|---|---|---|---|---|---|
| PRT | CDS | a-1 | a-2 | b-4 | i-2 | i-3 |
| 36517 | 36549, | 36677, 36709, | 37061, 37093, | 37445, 37477, | | 37829, 37861, |
| | 36581, | 36741, 36773, | 37125, 37157, | 37509, 37541, | | 37893, 37925, |
| | 36613, | 36805, 36837, | 37189, 37221, | 37573, 37605, | | 37957, 37989, |
| | 36645 | 36869, 36901, | 37253, 37285, | 37637, 37669, | | 38021, 38053, |
| | | 36933, 36965, | 37317, 37349, | 37701, 37733, | | 38085, 38117, |
| | | 36997, 37029 | 37381, 37413 | 37765, 37797 | | 38149, 38181 |
| 36518 | 36550, | 36678, 36710, | 37062, 37094, | 37446, 37478, | | 37830, 37862, |
| | 36582, | 36742, 36774, | 37126, 37158, | 37510, 37542, | | 37894, 37926, |
| | 36614, | 36806, 36838, | 37190, 37222, | 37574, 37606, | | 37958, 37990, |
| | 36646 | 36870, 36902, | 37254, 37286, | 37638, 37670, | | 38022, 38054, |
| | | 36934, 36966, | 37318, 37350, | 37702, 37734, | | 38086, 38118, |
| | | 36998, 37030 | 37382, 37414 | 37766, 37798 | | 38150, 38182 |
| 36519 | 36551, | 36679, 36711, | 37063, 37095, | 37447, 37479, | | 37831, 37863, |
| | 36583, | 36743, 36775, | 37127, 37159, | 37511, 37543, | | 37895, 37927, |
| | 36615, | 36807, 36839, | 37191, 37223, | 37575, 37607, | | 37959, 37991, |
| | 36647 | 36871, 36903, | 37255, 37287, | 37639, 37671, | | 38023, 38055, |
| | | 36935, 36967, | 37319, 37351, | 37703, 37735, | | 38087, 38119, |
| | | 36999, 37031 | 37383, 37415 | 37767, 37799 | | 38151, 38183 |
| 36520 | 36552, | 36680, 36712, | 37064, 37096, | 37448, 37480, | | 37832, 37864, |
| | 36584, | 36744, 36776, | 37128, 37160, | 37512, 37544, | | 37896, 37928, |
| | 36616, | 36808, 36840, | 37192, 37224, | 37576, 37608, | | 37960, 37992, |
| | 36648 | 36872, 36904, | 37256, 37288, | 37640, 37672, | | 38024, 38056, |
| | | 36936, 36968, | 37320, 37352, | 37704, 37736, | | 38088, 38120, |
| | | 37000, 37032 | 37384, 37416 | 37768, 37800 | | 38152, 38184 |
| 36521 | 36553, | 36681, 36713, | 37065, 37097, | 37449, 37481, | | 37833, 37865, |
| | 36585, | 36745, 36777, | 37129, 37161, | 37513, 37545, | | 37897, 37929, |
| | 36617, | 36809, 36841, | 37193, 37225, | 37577, 37609, | | 37961, 37993, |
| | 36649 | 36873, 36905, | 37257, 37289, | 37641, 37673, | | 38025, 38057, |
| | | 36937, 36969, | 37321, 37353, | 37705, 37737, | | 38089, 38121, |
| | | 37001, 37033 | 37385, 37417 | 37769, 37801 | | 38153, 38185 |
| 36522 | 36554, | 36682, 36714, | 37066, 37098, | 37450, 37482, | | 37834, 37866, |
| | 36586, | 36746, 36778, | 37130, 37162, | 37514, 37546, | | 37898, 37930, |
| | 36618, | 36810, 36842, | 37194, 37226, | 37578, 37610, | | 37962, 37994, |
| | 36650 | 36874, 36906, | 37258, 37290, | 37642, 37674, | | 38026, 38058, |
| | | 36938, 36970, | 37322, 37354, | 37706, 37738, | | 38090, 38122, |
| | | 37002, 37034 | 37386, 37418 | 37770, 37802 | | 38154, 38186 |
| 36523 | 36555, | 36683, 36715, | 37067, 37099, | 37451, 37483, | | 37835, 37867, |
| | 36587, | 36747, 36779, | 37131, 37163, | 37515, 37547, | | 37899, 37931, |
| | 36619, | 36811, 36843, | 37195, 37227, | 37579, 37611, | | 37963, 37995, |
| | 36651 | 36875, 36907, | 37259, 37291, | 37643, 37675, | | 38027, 38059, |
| | | 36939, 36971, | 37323, 37355, | 37707, 37739, | | 38091, 38123, |
| | | 37003, 37035 | 37387, 37419 | 37771, 37803 | | 38155, 38187 |
| 36524 | 36556, | 36684, 36716, | 37068, 37100, | 37452, 37484, | | 37836, 37868, |
| | 36588, | 36748, 36780, | 37132, 37164, | 37516, 37548, | | 37900, 37932, |
| | 36620, | 36812, 36844, | 37196, 37228, | 37580, 37612, | | 37964, 37996, |
| | 36652 | 36876, 36908, | 37260, 37292, | 37644, 37676, | | 38028, 38060, |
| | | 36940, 36972, | 37324, 37356, | 37708, 37740, | | 38092, 38124, |
| | | 37004, 37036 | 37388, 37420 | 37772, 37804 | | 38156, 38188 |
| 36525 | 36557, | 36685, 36717, | 37069, 37101, | 37453, 37485, | | 37837, 37869, |
| | 36589, | 36749, 36781, | 37133, 37165, | 37517, 37549, | | 37901, 37933, |
| | 36621, | 36813, 36845, | 37197, 37229, | 37581, 37613, | | 37965, 37997, |
| | 36653 | 36877, 36909, | 37261, 37293, | 37645, 37677, | | 38029, 38061, |
| | | 36941, 36973, | 37325, 37357, | 37709, 37741, | | 38093, 38125, |
| | | 37005, 37037 | 37389, 37421 | 37773, 37805 | | 38157, 38189 |
| 36526 | 36558, | 36686, 36718, | 37070, 37102, | 37454, 37486, | | 37838, 37870, |
| | 36590, | 36750, 36782, | 37134, 37166, | 37518, 37550, | | 37902, 37934, |
| | 36622, | 36814, 36846, | 37198, 37230, | 37582, 37614, | | 37966, 37998, |
| | 36654 | 36878, 36910, | 37262, 37294, | 37646, 37678, | | 38030, 38062, |
| | | 36942, 36974, | 37326, 37358, | 37710, 37742, | | 38094, 38126, |
| | | 37006, 37038 | 37390, 37422 | 37774, 37806 | | 38158, 38190 |
| 36527 | 36559, | 36687, 36719, | 37071, 37103, | 37455, 37487, | | 37839, 37871, |
| | 36591, | 36751, 36783, | 37135, 37167, | 37519, 37551, | | 37903, 37935, |
| | 36623, | 36815, 36847, | 37199, 37231, | 37583, 37615, | | 37967, 37999, |
| | 36655 | 36879, 36911, | 37263, 37295, | 37647, 37679, | | 38031, 38063, |
| | | 36943, 36975, | 37327, 37359, | 37711, 37743, | | 38095, 38127, |
| | | 37007, 37039 | 37391, 37423 | 37775, 37807 | | 38159, 38191 |
| 36528 | 36560, | 36688, 36720, | 37072, 37104, | 37456, 37488, | | 37840, 37872, |
| | 36592, | 36752, 36784, | 37136, 37168, | 37520, 37552, | | 37904, 37936, |
| | 36624, | 36816, 36848, | 37200, 37232, | 37584, 37616, | | 37968, 38000, |
| | 36656 | 36880, 36912, | 37264, 37296, | 37648, 37680, | | 38032, 38064, |
| | | 36944, 36976, | 37328, 37360, | 37712, 37744, | | 38096, 38128, |
| | | 37008, 37040 | 37392, 37424 | 37776, 37808 | | 38160, 38192 |
| 36529 | 36561, | 36689, 36721, | 37073, 37105, | 37457, 37489, | | 37841, 37873, |
| | 36593, | 36753, 36785, | 37137, 37169, | 37521, 37553, | | 37905, 37937, |
| | 36625, | 36817, 36849, | 37201, 37233, | 37585, 37617, | | 37969, 38001, |
| | 36657 | 36881, 36913, | 37265, 37297, | 37649, 37681, | | 38033, 38065, |
| | | 36945, 36977, | 37329, 37361, | 37713, 37745, | | 38097, 38129, |
| | | 37009, 37041 | 37393, 37425 | 37777, 37809 | | 38161, 38193 |
| 36530 | 36562, | 36690, 36722, | 37074, 37106, | 37458, 37490, | | 37842, 37874, |
| | 36594, | 36754, 36786, | 37138, 37170, | 37522, 37554, | | 37906, 37938, |
| | 36626, | 36818, 36850, | 37202, 37234, | 37586, 37618, | | 37970, 38002, |
| | 36658 | 36882, 36914, | 37266, 37298, | 37650, 37682, | | 38034, 38066, |
| | | 36946, 36978, | 37330, 37362, | 37714, 37746, | | 38098, 38130, |
| | | 37010, 37042 | 37394, 37426 | 37778, 37810 | | 38162, 38194 |
| 36531 | 36563, | 36691, 36723, | 37075, 37107, | 37459, 37491, | | 37843, 37875, |
| | 36595, | 36755, 36787, | 37139, 37171, | 37523, 37555, | | 37907, 37939, |
| | 36627, | 36819, 36851, | 37203, 37235, | 37587, 37619, | | 37971, 38003, |
| | 36659 | 36883, 36915, | 37267, 37299, | 37651, 37683, | | 38035, 38067, |
| | | 36947, 36979, | 37331, 37363, | 37715, 37747, | | 38099, 38131, |
| | | 37011, 37043 | 37395, 37427 | 37779, 37811 | | 38163, 38195 |
| 36532 | 36564, | 36692, 36724, | 37076, 37108, | 37460, 37492, | | 37844, 37876, |
| | 36596, | 36756, 36788, | 37140, 37172, | 37524, 37556, | | 37908, 37940, |
| | 36628, | 36820, 36852, | 37204, 37236, | 37588, 37620, | | 37972, 38004, |
| | 36660 | 36884, 36916, | 37268, 37300, | 37652, 37684, | | 38036, 38068, |
| | | 36948, 36980, | 37332, 37364, | 37716, 37748, | | 38100, 38132, |
| | | 37012, 37044 | 37396, 37428 | 37780, 37812 | | 38164, 38196 |
| 36533 | 36565, | 36693, 36725, | 37077, 37109, | 37461, 37493, | | 37845, 37877, |
| | 36597, | 36757, 36789, | 37141, 37173, | 37525, 37557, | | 37909, 37941, |
| | 36629, | 36821, 36853, | 37205, 37237, | 37589, 37621, | | 37973, 38005, |
| | 36661 | 36885, 36917, | 37269, 37301, | 37653, 37685, | | 38037, 38069, |
| | | 36949, 36981, | 37333, 37365, | 37717, 37749, | | 38101, 38133, |
| | | 37013, 37045 | 37397, 37429 | 37781, 37813 | | 38165, 38197 |
| 36534 | 36566, | 36694, 36726, | 37078, 37110, | 37462, 37494, | | 37846, 37878, |
| | 36598, | 36758, 36790, | 37142, 37174, | 37526, 37558, | | 37910, 37942, |
| | 36630, | 36822, 36854, | 37206, 37238, | 37590, 37622, | | 37974, 38006, |
| | 36662 | 36886, 36918, | 37270, 37302, | 37654, 37686, | | 38038, 38070, |
| | | 36950, 36982, | 37334, 37366, | 37718, 37750, | | 38102, 38134, |
| | | 37014, 37046 | 37398, 37430 | 37782, 37814 | | 38166, 38198 |
| 36535 | 36567, | 36695, 36727, | 37079, 37111, | 37463, 37495, | | 37847, 37879, |
| | 36599, | 36759, 36791, | 37143, 37175, | 37527, 37559, | | 37911, 37943, |
| | 36631, | 36823, 36855, | 37207, 37239, | 37591, 37623, | | 37975, 38007, |
| | 36663 | 36887, 36919, | 37271, 37303, | 37655, 37687, | | 38039, 38071, |
| | | 36951, 36983, | 37335, 37367, | 37719, 37751, | | 38103, 38135, |
| | | 37015, 37047 | 37399, 37431 | 37783, 37815 | | 38167, 38199 |
| 36536 | 36568, | 36696, 36728, | 37080, 37112, | 37464, 37496, | | 37848, 37880, |
| | 36600, | 36760, 36792, | 37144, 37176, | 37528, 37560, | | 37912, 37944, |
| | 36632, | 36824, 36856, | 37208, 37240, | 37592, 37624, | | 37976, 38008, |
| | 36664 | 36888, 36920, | 37272, 37304, | 37656, 37688, | | 38040, 38072, |
| | | 36952, 36984, | 37336, 37368, | 37720, 37752, | | 38104, 38136, |
| | | 37016, 37048 | 37400, 37432 | 37784, 37816 | | 38168, 38200 |
| 36537 | 36569, | 36697, 36729, | 37081, 37113, | 37465, 37497, | | 37849, 37881, |
| | 36601, | 36761, 36793, | 37145, 37177, | 37529, 37561, | | 37913, 37945, |
| | 36633, | 36825, 36857, | 37209, 37241, | 37593, 37625, | | 37977, 38009, |
| | 36665 | 36889, 36921, | 37273, 37305, | 37657, 37689, | | 38041, 38073, |
| | | 36953, 36985, | 37337, 37369, | 37721, 37753, | | 38105, 38137, |
| | | 37017, 37049 | 37401, 37433 | 37785, 37817 | | 38169, 38201 |
| 36538 | 36570, | 36698, 36730, | 37082, 37114, | 37466, 37498, | | 37850, 37882, |
| | 36602, | 36762, 36794, | 37146, 37178, | 37530, 37562, | | 37914, 37946, |
| | 36634, | 36826, 36858, | 37210, 37242, | 37594, 37626, | | 37978, 38010, |
| | 36666 | 36890, 36922, | 37274, 37306, | 37658, 37690, | | 38042, 38074, |
| | | 36954, 36986, | 37338, 37370, | 37722, 37754, | | 38106, 38138, |
| | | 37018, 37050 | 37402, 37434 | 37786, 37818 | | 38170, 38202 |
| 36539 | 36571, | 36699, 36731, | 37083, 37115, | 37467, 37499, | | 37851, 37883, |
| | 36603, | 36763, 36795, | 37147, 37179, | 37531, 37563, | | 37915, 37947, |
| | 36635, | 36827, 36859, | 37211, 37243, | 37595, 37627, | | 37979, 38011, |
| | 36667 | 36891, 36923, | 37275, 37307, | 37659, 37691, | | 38043, 38075, |
| | | 36955, 36987, | 37339, 37371, | 37723, 37755, | | 38107, 38139, |
| | | 37019, 37051 | 37403, 37435 | 37787, 37819 | | 38171, 38203 |
| 36540 | 36572, | 36700, 36732, | 37084, 37116, | 37468, 37500, | | 37852, 37884, |
| | 36604, | 36764, 36796, | 37148, 37180, | 37532, 37564, | | 37916, 37948, |
| | 36636, | 36828, 36860, | 37212, 37244, | 37596, 37628, | | 37980, 38012, |
| | 36668 | 36892, 36924, | 37276, 37308, | 37660, 37692, | | 38044, 38076, |
| | | 36956, 36988, | 37340, 37372, | 37724, 37756, | | 38108, 38140, |
| | | 37020, 37052 | 37404, 37436 | 37788, 37820 | | 38172, 38204 |
| 36541 | 36573, | 36701, 36733, | 37085, 37117, | 37469, 37501, | | 37853, 37885, |
| | 36605, | 36765, 36797, | 37149, 37181, | 37533, 37565, | | 37917, 37949, |
| | 36637, | 36829, 36861, | 37213, 37245, | 37597, 37629, | | 37981, 38013, |
| | 36669 | 36893, 36925, | 37277, 37309, | 37661, 37693, | | 38045, 38077, |
| | | 36957, 36989, | 37341, 37373, | 37725, 37757, | | 38109, 38141, |
| | | 37021, 37053 | 37405, 37437 | 37789, 37821 | | 38173, 38205 |
| 36542 | 36574, | 36702, 36734, | 37086, 37118, | 37470, 37502, | | 37854, 37886, |
| | 36606, | 36766, 36798, | 37150, 37182, | 37534, 37566, | | 37918, 37950, |
| | 36638, | 36830, 36862, | 37214, 37246, | 37598, 37630, | | 37982, 38014, |
| | 36670 | 36894, 36926, | 37278, 37310, | 37662, 37694, | | 38046, 38078, |
| | | 36958, 36990, | 37342, 37374, | 37726, 37758, | | 38110, 38142, |
| | | 37022, 37054 | 37406, 37438 | 37790, 37822 | | 38174, 38206 |
| 36543 | 36575, | 36703, 36735, | 37087, 37119, | 37471, 37503, | | 37855, 37887, |
| | 36607, | 36767, 36799, | 37151, 37183, | 37535, 37567, | | 37919, 37951, |
| | 36639, | 36831, 36863, | 37215, 37247, | 37599, 37631, | | 37983, 38015, |
| | 36671 | 36895, 36927, | 37279, 37311, | 37663, 37695, | | 38047, 38079, |
| | | 36959, 36991, | 37343, 37375, | 37727, 37759, | | 38111, 38143, |
| | | 37023, 37055 | 37407, 37439 | 37791, 37823 | | 38175, 38207 |
| 36544 | 36576, | 36704, 36736, | 37088, 37120, | 37472, 37504, | | 37856, 37888, |
| | 36608, | 36768, 36800, | 37152, 37184, | 37536, 37568, | | 37920, 37952, |
| | 36640, | 36832, 36864, | 37216, 37248, | 37600, 37632, | | 37984, 38016, |
| | 36672 | 36896, 36928, | 37280, 37312, | 37664, 37696, | | 38048, 38080, |
| | | 36960, 36992, | 37344, 37376, | 37728, 37760, | | 38112, 38144, |
| | | 37024, 37056 | 37408, 37440 | 37792, 37824 | | 38176, 38208 |
| 36545 | 36577, | 36705, 36737, | 37089, 37121, | 37473, 37505, | | 37857, 37889, |
| | 36609, | 36769, 36801, | 37153, 37185, | 37537, 37569, | | 37921, 37953, |
| | 36641, | 36833, 36865, | 37217, 37249, | 37601, 37633, | | 37985, 38017, |
| | 36673 | 36897, 36929, | 37281, 37313, | 37665, 37697, | | 38049, 38081, |
| | | 36961, 36993, | 37345, 37377, | 37729, 37761, | | 38113, 38145, |
| | | 37025, 37057 | 37409, 37441 | 37793, 37825 | | 38177, 38209 |
| 36546 | 36578, | 36706, 36738, | 37090, 37122, | 37474, 37506, | | 37858, 37890, |
| | 36610, | 36770, 36802, | 37154, 37186, | 37538, 37570, | | 37922, 37954, |
| | 36642, | 36834, 36866, | 37218, 37250, | 37602, 37634, | | 37986, 38018, |
| | 36674 | 36898, 36930, | 37282, 37314, | 37666, 37698, | | 38050, 38082, |
| | | 36962, 36994, | 37346, 37378, | 37730, 37762, | | 38114, 38146, |
| | | 37026, 37058 | 37410, 37442 | 37794, 37826 | | 38178, 38210 |
| 36547 | 36579, | 36707, 36739, | 37091, 37123, | 37475, 37507, | | 37859, 37891, |
| | 36611, | 36771, 36803, | 37155, 37187, | 37539, 37571, | | 37923, 37955, |
| | 36643, | 36835, 36867, | 37219, 37251, | 37603, 37635, | | 37987, 38019, |
| | 36675 | 36899, 36931, | 37283, 37315, | 37667, 37699, | | 38051, 38083, |
| | | 36963, 36995, | 37347, 37379, | 37731, 37763, | | 38115, 38147, |
| | | 37027, 37059 | 37411, 37443 | 37795, 37827 | | 38179, 38211 |
| 36548 | 36580, | 36708, 36740, | 37092, 37124, | 37476, 37508, | | 37860, 37892, |
| | 36612, | 36772, 36804, | 37156, 37188, | 37540, 37572, | | 37924, 37956, |
| | 36644, | 36836, 36868, | 37220, 37252, | 37604, 37636, | | 37988, 38020, |
| | 36676 | 36900, 36932, | 37284, 37316, | 37668, 37700, | | 38052, 38084, |
| | | 36964, 36996, | 37348, 37380, | 37732, 37764, | | 38116, 38148, |
| | | 37028, 37060 | 37412, 37444 | 37796, 37828 | | 38180, 38212 |

TABLE 3.11
Preferred LASV prefusion-stabilized (GPCmut11) constructs

| | | RNA designs and SEQ ID NOs | | | | |
|---|---|---|---|---|---|---|
| PRT | CDS | a-1 | a-2 | b-4 | i-2 | i-3 |
| 38213 | 38245, | 38373, 38405, | 38757, 38789, | 39141, 39173, | | 39525, 39557, |
| | 38277, | 38437, 38469, | 38821, 38853, | 39205, 39237, | | 39589, 39621, |
| | 38309, | 38501, 38533, | 38885, 38917, | 39269, 39301, | | 39653, 39685, |
| | 38341 | 38565, 38597, | 38949, 38981, | 39333, 39365, | | 39717, 39749, |
| | | 38629, 38661, | 39013, 39045, | 39397, 39429, | | 39781, 39813, |
| | | 38693, 38725 | 39077, 39109 | 39461, 39493 | | 39845, 39877 |
| 38214 | 38246, | 38374, 38406, | 38758, 38790, | 39142, 39174, | | 39526, 39558, |
| | 38278, | 38438, 38470, | 38822, 38854, | 39206, 39238, | | 39590, 39622, |
| | 38310, | 38502, 38534, | 38886, 38918, | 39270, 39302, | | 39654, 39686, |
| | 38342 | 38566, 38598, | 38950, 38982, | 39334, 39366, | | 39718, 39750, |
| | | 38630, 38662, | 39014, 39046, | 39398, 39430, | | 39782, 39814, |
| | | 38694, 38726 | 39078, 39110 | 39462, 39494 | | 39846, 39878 |
| 38215 | 38247, | 38375, 38407, | 38759, 38791, | 39143, 39175, | | 39527, 39559, |
| | 38279, | 38439, 38471, | 38823, 38855, | 39207, 39239, | | 39591, 39623, |
| | 38311, | 38503, 38535, | 38887, 38919, | 39271, 39303, | | 39655, 39687, |
| | 38343 | 38567, 38599, | 38951, 38983, | 39335, 39367, | | 39719, 39751, |
| | | 38631, 38663, | 39015, 39047, | 39399, 39431, | | 39783, 39815, |
| | | 38695, 38727 | 39079, 39111 | 39463, 39495 | | 39847, 39879 |
| 38216 | 38248, | 38376, 38408, | 38760, 38792, | 39144, 39176, | | 39528, 39560, |
| | 38280, | 38440, 38472, | 38824, 38856, | 39208, 39240, | | 39592, 39624, |
| | 38312, | 38504, 38536, | 38888, 38920, | 39272, 39304, | | 39656, 39688, |
| | 38344 | 38568, 38600, | 38952, 38984, | 39336, 39368, | | 39720, 39752, |
| | | 38632, 38664, | 39016, 39048, | 39400, 39432, | | 39784, 39816, |
| | | 38696, 38728 | 39080, 39112 | 39464, 39496 | | 39848, 39880 |
| 38217 | 38249, | 38377, 38409, | 38761, 38793, | 39145, 39177, | | 39529, 39561, |
| | 38281, | 38441, 38473, | 38825, 38857, | 39209, 39241, | | 39593, 39625, |
| | 38313, | 38505, 38537, | 38889, 38921, | 39273, 39305, | | 39657, 39689, |
| | 38345 | 38569, 38601, | 38953, 38985, | 39337, 39369, | | 39721, 39753, |
| | | 38633, 38665, | 39017, 39049, | 39401, 39433, | | 39785, 39817, |
| | | 38697, 38729 | 39081, 39113 | 39465, 39497 | | 39849, 39881 |
| 38218 | 38250, | 38378, 38410, | 38762, 38794, | 39146, 39178, | | 39530, 39562, |
| | 38282, | 38442, 38474, | 38826, 38858, | 39210, 39242, | | 39594, 39626, |
| | 38314, | 38506, 38538, | 38890, 38922, | 39274, 39306, | | 39658, 39690, |
| | 38346 | 38570, 38602, | 38954, 38986, | 39338, 39370, | | 39722, 39754, |
| | | 38634, 38666, | 39018, 39050, | 39402, 39434, | | 39786, 39818, |
| | | 38698, 38730 | 39082, 39114 | 39466, 39498 | | 39850, 39882 |
| 38219 | 38251, | 38379, 38411, | 38763, 38795, | 39147, 39179, | | 39531, 39563, |
| | 38283, | 38443, 38475, | 38827, 38859, | 39211, 39243, | | 39595, 39627, |
| | 38315, | 38507, 38539, | 38891, 38923, | 39275, 39307, | | 39659, 39691, |
| | 38347 | 38571, 38603, | 38955, 38987, | 39339, 39371, | | 39723, 39755, |
| | | 38635, 38667, | 39019, 39051, | 39403, 39435, | | 39787, 39819, |
| | | 38699, 38731 | 39083, 39115 | 39467, 39499 | | 39851, 39883 |
| 38220 | 38252, | 38380, 38412, | 38764, 38796, | 39148, 39180, | | 39532, 39564, |
| | 38284, | 38444, 38476, | 38828, 38860, | 39212, 39244, | | 39596, 39628, |
| | 38316, | 38508, 38540, | 38892, 38924, | 39276, 39308, | | 39660, 39692, |
| | 38348 | 38572, 38604, | 38956, 38988, | 39340, 39372, | | 39724, 39756, |
| | | 38636, 38668, | 39020, 39052, | 39404, 39436, | | 39788, 39820, |
| | | 38700, 38732 | 39084, 39116 | 39468, 39500 | | 39852, 39884 |
| 38221 | 38253, | 38381, 38413, | 38765, 38797, | 39149, 39181, | | 39533, 39565, |
| | 38285, | 38445, 38477, | 38829, 38861, | 39213, 39245, | | 39597, 39629, |
| | 38317, | 38509, 38541, | 38893, 38925, | 39277, 39309, | | 39661, 39693, |
| | 38349 | 38573, 38605, | 38957, 38989, | 39341, 39373, | | 39725, 39757, |
| | | 38637, 38669, | 39021, 39053, | 39405, 39437, | | 39789, 39821, |
| | | 38701, 38733 | 39085, 39117 | 39469, 39501 | | 39853, 39885 |
| 38222 | 38254, | 38382, 38414, | 38766, 38798, | 39150, 39182, | | 39534, 39566, |
| | 38286, | 38446, 38478, | 38830, 38862, | 39214, 39246, | | 39598, 39630, |
| | 38318, | 38510, 38542, | 38894, 38926, | 39278, 39310, | | 39662, 39694, |
| | 38350 | 38574, 38606, | 38958, 38990, | 39342, 39374, | | 39726, 39758, |
| | | 38638, 38670, | 39022, 39054, | 39406, 39438, | | 39790, 39822, |
| | | 38702, 38734 | 39086, 39118 | 39470, 39502 | | 39854, 39886 |
| 38223 | 38255, | 38383, 38415, | 38767, 38799, | 39151, 39183, | | 39535, 39567, |
| | 38287, | 38447, 38479, | 38831, 38863, | 39215, 39247, | | 39599, 39631, |
| | 38319, | 38511, 38543, | 38895, 38927, | 39279, 39311, | | 39663, 39695, |
| | 38351 | 38575, 38607, | 38959, 38991, | 39343, 39375, | | 39727, 39759, |
| | | 38639, 38671, | 39023, 39055, | 39407, 39439, | | 39791, 39823, |
| | | 38703, 38735 | 39087, 39119 | 39471, 39503 | | 39855, 39887 |
| 38224 | 38256, | 38384, 38416, | 38768, 38800, | 39152, 39184, | | 39536, 39568, |
| | 38288, | 38448, 38480, | 38832, 38864, | 39216, 39248, | | 39600, 39632, |
| | 38320, | 38512, 38544, | 38896, 38928, | 39280, 39312, | | 39664, 39696, |
| | 38352 | 38576, 38608, | 38960, 38992, | 39344, 39376, | | 39728, 39760, |
| | | 38640, 38672, | 39024, 39056, | 39408, 39440, | | 39792, 39824, |
| | | 38704, 38736 | 39088, 39120 | 39472, 39504 | | 39856, 39888 |
| 38225 | 38257, | 38385, 38417, | 38769, 38801, | 39153, 39185, | | 39537, 39569, |
| | 38289, | 38449, 38481, | 38833, 38865, | 39217, 39249, | | 39601, 39633, |
| | 38321, | 38513, 38545, | 38897, 38929, | 39281, 39313, | | 39665, 39697, |
| | 38353 | 38577, 38609, | 38961, 38993, | 39345, 39377, | | 39729, 39761, |
| | | 38641, 38673, | 39025, 39057, | 39409, 39441, | | 39793, 39825, |
| | | 38705, 38737 | 39089, 39121 | 39473, 39505 | | 39857, 39889 |
| 38226 | 38258, | 38386, 38418, | 38770, 38802, | 39154, 39186, | | 39538, 39570, |
| | 38290, | 38450, 38482, | 38834, 38866, | 39218, 39250, | | 39602, 39634, |
| | 38322, | 38514, 38546, | 38898, 38930, | 39282, 39314, | | 39666, 39698, |
| | 38354 | 38578, 38610, | 38962, 38994, | 39346, 39378, | | 39730, 39762, |
| | | 38642, 38674, | 39026, 39058, | 39410, 39442, | | 39794, 39826, |
| | | 38706, 38738 | 39090, 39122 | 39474, 39506 | | 39858, 39890 |
| 38227 | 38259, | 38387, 38419, | 38771, 38803, | 39155, 39187, | | 39539, 39571, |
| | 38291, | 38451, 38483, | 38835, 38867, | 39219, 39251, | | 39603, 39635, |
| | 38323, | 38515, 38547, | 38899, 38931, | 39283, 39315, | | 39667, 39699, |
| | 38355 | 38579, 38611, | 38963, 38995, | 39347, 39379, | | 39731, 39763, |
| | | 38643, 38675, | 39027, 39059, | 39411, 39443, | | 39795, 39827, |
| | | 38707, 38739 | 39091, 39123 | 39475, 39507 | | 39859, 39891 |
| 38228 | 38260, | 38388, 38420, | 38772, 38804, | 39156, 39188, | | 39540, 39572, |
| | 38292, | 38452, 38484, | 38836, 38868, | 39220, 39252, | | 39604, 39636, |
| | 38324, | 38516, 38548, | 38900, 38932, | 39284, 39316, | | 39668, 39700, |
| | 38356 | 38580, 38612, | 38964, 38996, | 39348, 39380, | | 39732, 39764, |
| | | 38644, 38676, | 39028, 39060, | 39412, 39444, | | 39796, 39828, |
| | | 38708, 38740 | 39092, 39124 | 39476, 39508 | | 39860, 39892 |
| 38229 | 38261, | 38389, 38421, | 38773, 38805, | 39157, 39189, | | 39541, 39573, |
| | 38293, | 38453, 38485, | 38837, 38869, | 39221, 39253, | | 39605, 39637, |
| | 38325, | 38517, 38549, | 38901, 38933, | 39285, 39317, | | 39669, 39701, |
| | 38357 | 38581, 38613, | 38965, 38997, | 39349, 39381, | | 39733, 39765, |
| | | 38645, 38677, | 39029, 39061, | 39413, 39445, | | 39797, 39829, |
| | | 38709, 38741 | 39093, 39125 | 39477, 39509 | | 39861, 39893 |
| 38230 | 38262, | 38390, 38422, | 38774, 38806, | 39158, 39190, | | 39542, 39574, |
| | 38294, | 38454, 38486, | 38838, 38870, | 39222, 39254, | | 39606, 39638, |
| | 38326, | 38518, 38550, | 38902, 38934, | 39286, 39318, | | 39670, 39702, |
| | 38358 | 38582, 38614, | 38966, 38998, | 39350, 39382, | | 39734, 39766, |
| | | 38646, 38678, | 39030, 39062, | 39414, 39446, | | 39798, 39830, |
| | | 38710, 38742 | 39094, 39126 | 39478, 39510 | | 39862, 39894 |
| 38231 | 38263, | 38391, 38423, | 38775, 38807, | 39159, 39191, | | 39543, 39575, |
| | 38295, | 38455, 38487, | 38839, 38871, | 39223, 39255, | | 39607, 39639, |
| | 38327, | 38519, 38551, | 38903, 38935, | 39287, 39319, | | 39671, 39703, |
| | 38359 | 38583, 38615, | 38967, 38999, | 39351, 39383, | | 39735, 39767, |
| | | 38647, 38679, | 39031, 39063, | 39415, 39447, | | 39799, 39831, |
| | | 38711, 38743 | 39095, 39127 | 39479, 39511 | | 39863, 39895 |
| 38232 | 38264, | 38392, 38424, | 38776, 38808, | 39160, 39192, | | 39544, 39576, |
| | 38296, | 38456, 38488, | 38840, 38872, | 39224, 39256, | | 39608, 39640, |
| | 38328, | 38520, 38552, | 38904, 38936, | 39288, 39320, | | 39672, 39704, |
| | 38360 | 38584, 38616, | 38968, 39000, | 39352, 39384, | | 39736, 39768, |
| | | 38648, 38680, | 39032, 39064, | 39416, 39448, | | 39800, 39832, |
| | | 38712, 38744 | 39096, 39128 | 39480, 39512 | | 39864, 39896 |
| 38233 | 38265, | 38393, 38425, | 38777, 38809, | 39161, 39193, | | 39545, 39577, |
| | 38297, | 38457, 38489, | 38841, 38873, | 39225, 39257, | | 39609, 39641, |
| | 38329, | 38521, 38553, | 38905, 38937, | 39289, 39321, | | 39673, 39705, |
| | 38361 | 38585, 38617, | 38969, 39001, | 39353, 39385, | | 39737, 39769, |
| | | 38649, 38681, | 39033, 39065, | 39417, 39449, | | 39801, 39833, |
| | | 38713, 38745 | 39097, 39129 | 39481, 39513 | | 39865, 39897 |
| 38234 | 38266, | 38394, 38426, | 38778, 38810, | 39162, 39194, | | 39546, 39578, |
| | 38298, | 38458, 38490, | 38842, 38874, | 39226, 39258, | | 39610, 39642, |
| | 38330, | 38522, 38554, | 38906, 38938, | 39290, 39322, | | 39674, 39706, |
| | 38362 | 38586, 38618, | 38970, 39002, | 39354, 39386, | | 39738, 39770, |
| | | 38650, 38682, | 39034, 39066, | 39418, 39450, | | 39802, 39834, |
| | | 38714, 38746 | 39098, 39130 | 39482, 39514 | | 39866, 39898 |
| 38235 | 38267, | 38395, 38427, | 38779, 38811, | 39163, 39195, | | 39547, 39579, |
| | 38299, | 38459, 38491, | 38843, 38875, | 39227, 39259, | | 39611, 39643, |
| | 38331, | 38523, 38555, | 38907, 38939, | 39291, 39323, | | 39675, 39707, |
| | 38363 | 38587, 38619, | 38971, 39003, | 39355, 39387, | | 39739, 39771, |
| | | 38651, 38683, | 39035, 39067, | 39419, 39451, | | 39803, 39835, |
| | | 38715, 38747 | 39099, 39131 | 39483, 39515 | | 39867, 39899 |
| 38236 | 38268, | 38396, 38428, | 38780, 38812, | 39164, 39196, | | 39548, 39580, |
| | 38300, | 38460, 38492, | 38844, 38876, | 39228, 39260, | | 39612, 39644, |
| | 38332, | 38524, 38556, | 38908, 38940, | 39292, 39324, | | 39676, 39708, |
| | 38364 | 38588, 38620, | 38972, 39004, | 39356, 39388, | | 39740, 39772, |
| | | 38652, 38684, | 39036, 39068, | 39420, 39452, | | 39804, 39836, |
| | | 38716, 38748 | 39100, 39132 | 39484, 39516 | | 39868, 39900 |
| 38237 | 38269, | 38397, 38429, | 38781, 38813, | 39165, 39197, | | 39549, 39581, |
| | 38301, | 38461, 38493, | 38845, 38877, | 39229, 39261, | | 39613, 39645, |
| | 38333, | 38525, 38557, | 38909, 38941, | 39293, 39325, | | 39677, 39709, |
| | 38365 | 38589, 38621, | 38973, 39005, | 39357, 39389, | | 39741, 39773, |
| | | 38653, 38685, | 39037, 39069, | 39421, 39453, | | 39805, 39837, |
| | | 38717, 38749 | 39101, 39133 | 39485, 39517 | | 39869, 39901 |
| 38238 | 38270, | 38398, 38430, | 38782, 38814, | 39166, 39198, | | 39550, 39582, |
| | 38302, | 38462, 38494, | 38846, 38878, | 39230, 39262, | | 39614, 39646, |
| | 38334, | 38526, 38558, | 38910, 38942, | 39294, 39326, | | 39678, 39710, |
| | 38366 | 38590, 38622, | 38974, 39006, | 39358, 39390, | | 39742, 39774, |
| | | 38654, 38686, | 39038, 39070, | 39422, 39454, | | 39806, 39838, |
| | | 38718, 38750 | 39102, 39134 | 39486, 39518 | | 39870, 39902 |
| 38239 | 38271, | 38399, 38431, | 38783, 38815, | 39167, 39199, | | 39551, 39583, |
| | 38303, | 38463, 38495, | 38847, 38879, | 39231, 39263, | | 39615, 39647, |
| | 38335, | 38527, 38559, | 38911, 38943, | 39295, 39327, | | 39679, 39711, |
| | 38367 | 38591, 38623, | 38975, 39007, | 39359, 39391, | | 39743, 39775, |
| | | 38655, 38687, | 39039, 39071, | 39423, 39455, | | 39807, 39839, |
| | | 38719, 38751 | 39103, 39135 | 39487, 39519 | | 39871, 39903 |
| 38240 | 38272, | 38400, 38432, | 38784, 38816, | 39168, 39200, | | 39552, 39584, |
| | 38304, | 38464, 38496, | 38848, 38880, | 39232, 39264, | | 39616, 39648, |
| | 38336, | 38528, 38560, | 38912, 38944, | 39296, 39328, | | 39680, 39712, |
| | 38368 | 38592, 38624, | 38976, 39008, | 39360, 39392, | | 39744, 39776, |
| | | 38656, 38688, | 39040, 39072, | 39424, 39456, | | 39808, 39840, |
| | | 38720, 38752 | 39104, 39136 | 39488, 39520 | | 39872, 39904 |
| 38241 | 38273, | 38401, 38433, | 38785, 38817, | 39169, 39201, | | 39553, 39585, |
| | 38305, | 38465, 38497, | 38849, 38881, | 39233, 39265, | | 39617, 39649, |
| | 38337, | 38529, 38561, | 38913, 38945, | 39297, 39329, | | 39681, 39713, |
| | 38369 | 38593, 38625, | 38977, 39009, | 39361, 39393, | | 39745, 39777, |
| | | 38657, 38689, | 39041, 39073, | 39425, 39457, | | 39809, 39841, |
| | | 38721, 38753 | 39105, 39137 | 39489, 39521 | | 39873, 39905 |
| 38242 | 38274, | 38402, 38434, | 38786, 38818, | 39170, 39202, | | 39554, 39586, |
| | 38306, | 38466, 38498, | 38850, 38882, | 39234, 39266, | | 39618, 39650, |
| | 38338, | 38530, 38562, | 38914, 38946, | 39298, 39330, | | 39682, 39714, |
| | 38370 | 38594, 38626, | 38978, 39010, | 39362, 39394, | | 39746, 39778, |
| | | 38658, 38690, | 39042, 39074, | 39426, 39458, | | 39810, 39842, |
| | | 38722, 38754 | 39106, 39138 | 39490, 39522 | | 39874, 39906 |
| 38243 | 38275, | 38403, 38435, | 38787, 38819, | 39171, 39203, | | 39555, 39587, |
| | 38307, | 38467, 38499, | 38851, 38883, | 39235, 39267, | | 39619, 39651, |
| | 38339, | 38531, 38563, | 38915, 38947, | 39299, 39331, | | 39683, 39715, |
| | 38371 | 38595, 38627, | 38979, 39011, | 39363, 39395, | | 39747, 39779, |
| | | 38659, 38691, | 39043, 39075, | 39427, 39459, | | 39811, 39843, |
| | | 38723, 38755 | 39107, 39139 | 39491, 39523 | | 39875, 39907 |
| 38244 | 38276, | 38404, 38436, | 38788, 38820, | 39172, 39204, | | 39556, 39588, |
| | 38308, | 38468, 38500, | 38852, 38884, | 39236, 39268, | | 39620, 39652, |
| | 38340, | 38532, 38564, | 38916, 38948, | 39300, 39332, | | 39684, 39716, |
| | 38372 | 38596, 38628, | 38980, 39012, | 39364, 39396, | | 39748, 39780, |
| | | 38660, 38692, | 39044, 39076, | 39428, 39460, | | 39812, 39844, |
| | | 38724, 38756 | 39108, 39140 | 39492, 39524 | | 39876, 39908 |

TABLE 3.12
Preferred LASV prefusion-stabilized (GPCmut12) constructs

| | | RNA designs and SEQ ID NOs | | | | |
|---|---|---|---|---|---|---|
| PRT | CDS | a-1 | a-2 | b-4 | i-2 | i-3 |
| 39909 | 39941, | 40069, 40101, | 40453, 40485, | 40837, 40869, | | 41221, 41253, |
| | 39973, | 40133, 40165, | 40517, 40549, | 40901, 40933, | | 41285, 41317, |
| | 40005, | 40197, 40229, | 40581, 40613, | 40965, 40997, | | 41349, 41381, |
| | 40037 | 40261, 40293, | 40645, 40677, | 41029, 41061, | | 41413, 41445, |
| | | 40325, 40357, | 40709, 40741, | 41093, 41125, | | 41477, 41509, |
| | | 40389, 40421 | 40773, 40805 | 41157, 41189 | | 41541, 41573 |
| 39910 | 39942, | 40070, 40102, | 40454, 40486, | 40838, 40870, | | 41222, 41254, |
| | 39974, | 40134, 40166, | 40518, 40550, | 40902, 40934, | | 41286, 41318, |
| | 40006, | 40198, 40230, | 40582, 40614, | 40966, 40998, | | 41350, 41382, |
| | 40038 | 40262, 40294, | 40646, 40678, | 41030, 41062, | | 41414, 41446, |
| | | 40326, 40358, | 40710, 40742, | 41094, 41126, | | 41478, 41510, |
| | | 40390, 40422 | 40774, 40806 | 41158, 41190 | | 41542, 41574 |
| 39911 | 39943, | 40071, 40103, | 40455, 40487, | 40839, 40871, | | 41223, 41255, |
| | 39975, | 40135, 40167, | 40519, 40551, | 40903, 40935, | | 41287, 41319, |
| | 40007, | 40199, 40231, | 40583, 40615, | 40967, 40999, | | 41351, 41383, |
| | 40039 | 40263, 40295, | 40647, 40679, | 41031, 41063, | | 41415, 41447, |
| | | 40327, 40359, | 40711, 40743, | 41095, 41127, | | 41479, 41511, |
| | | 40391, 40423 | 40775, 40807 | 41159, 41191 | | 41543, 41575 |
| 39912 | 39944, | 40072, 40104, | 40456, 40488, | 40840, 40872, | | 41224, 41256, |
| | 39976, | 40136, 40168, | 40520, 40552, | 40904, 40936, | | 41288, 41320, |
| | 40008, | 40200, 40232, | 40584, 40616, | 40968, 41000, | | 41352, 41384, |
| | 40040 | 40264, 40296, | 40648, 40680, | 41032, 41064, | | 41416, 41448, |
| | | 40328, 40360, | 40712, 40744, | 41096, 41128, | | 41480, 41512, |
| | | 40392, 40424 | 40776, 40808 | 41160, 41192 | | 41544, 41576 |
| 39913 | 39945, | 40073, 40105, | 40457, 40489, | 40841, 40873, | | 41225, 41257, |
| | 39977, | 40137, 40169, | 40521, 40553, | 40905, 40937, | | 41289, 41321, |
| | 40009, | 40201, 40233, | 40585, 40617, | 40969, 41001, | | 41353, 41385, |
| | 40041 | 40265, 40297, | 40649, 40681, | 41033, 41065, | | 41417, 41449, |
| | | 40329, 40361, | 40713, 40745, | 41097, 41129, | | 41481, 41513, |
| | | 40393, 40425 | 40777, 40809 | 41161, 41193 | | 41545, 41577 |
| 39914 | 39946, | 40074, 40106, | 40458, 40490, | 40842, 40874, | | 41226, 41258, |
| | 39978, | 40138, 40170, | 40522, 40554, | 40906, 40938, | | 41290, 41322, |
| | 40010, | 40202, 40234, | 40586, 40618, | 40970, 41002, | | 41354, 41386, |
| | 40042 | 40266, 40298, | 40650, 40682, | 41034, 41066, | | 41418, 41450, |
| | | 40330, 40362, | 40714, 40746, | 41098, 41130, | | 41482, 41514, |
| | | 40394, 40426 | 40778, 40810 | 41162, 41194 | | 41546, 41578 |
| 39915 | 39947, | 40075, 40107, | 40459, 40491, | 40843, 40875, | | 41227, 41259, |
| | 39979, | 40139, 40171, | 40523, 40555, | 40907, 40939, | | 41291, 41323, |
| | 40011, | 40203, 40235, | 40587, 40619, | 40971, 41003, | | 41355, 41387, |
| | 40043 | 40267, 40299, | 40651, 40683, | 41035, 41067, | | 41419, 41451, |
| | | 40331, 40363, | 40715, 40747, | 41099, 41131, | | 41483, 41515, |
| | | 40395, 40427 | 40779, 40811 | 41163, 41195 | | 41547, 41579 |
| 39916 | 39948, | 40076, 40108, | 40460, 40492, | 40844, 40876, | | 41228, 41260, |
| | 39980, | 40140, 40172, | 40524, 40556, | 40908, 40940, | | 41292, 41324, |
| | 40012, | 40204, 40236, | 40588, 40620, | 40972, 41004, | | 41356, 41388, |
| | 40044 | 40268, 40300, | 40652, 40684, | 41036, 41068, | | 41420, 41452, |
| | | 40332, 40364, | 40716, 40748, | 41100, 41132, | | 41484, 41516, |
| | | 40396, 40428 | 40780, 40812 | 41164, 41196 | | 41548, 41580 |
| 39917 | 39949, | 40077, 40109, | 40461, 40493, | 40845, 40877, | | 41229, 41261, |
| | 39981, | 40141, 40173, | 40525, 40557, | 40909, 40941, | | 41293, 41325, |
| | 40013, | 40205, 40237, | 40589, 40621, | 40973, 41005, | | 41357, 41389, |
| | 40045 | 40269, 40301, | 40653, 40685, | 41037, 41069, | | 41421, 41453, |
| | | 40333, 40365, | 40717, 40749, | 41101, 41133, | | 41485, 41517, |
| | | 40397, 40429 | 40781, 40813 | 41165, 41197 | | 41549, 41581 |
| 39918 | 39950, | 40078, 40110, | 40462, 40494, | 40846, 40878, | | 41230, 41262, |
| | 39982, | 40142, 40174, | 40526, 40558, | 40910, 40942, | | 41294, 41326, |
| | 40014, | 40206, 40238, | 40590, 40622, | 40974, 41006, | | 41358, 41390, |
| | 40046 | 40270, 40302, | 40654, 40686, | 41038, 41070, | | 41422, 41454, |
| | | 40334, 40366, | 40718, 40750, | 41102, 41134, | | 41486, 41518, |
| | | 40398, 40430 | 40782, 40814 | 41166, 41198 | | 41550, 41582 |
| 39919 | 39951, | 40079, 40111, | 40463, 40495, | 40847, 40879, | | 41231, 41263, |
| | 39983, | 40143, 40175, | 40527, 40559, | 40911, 40943, | | 41295, 41327, |
| | 40015, | 40207, 40239, | 40591, 40623, | 40975, 41007, | | 41359, 41391, |
| | 40047 | 40271, 40303, | 40655, 40687, | 41039, 41071, | | 41423, 41455, |
| | | 40335, 40367, | 40719, 40751, | 41103, 41135, | | 41487, 41519, |
| | | 40399, 40431 | 40783, 40815 | 41167, 41199 | | 41551, 41583 |
| 39920 | 39952, | 40080, 40112, | 40464, 40496, | 40848, 40880, | | 41232, 41264, |
| | 39984, | 40144, 40176, | 40528, 40560, | 40912, 40944, | | 41296, 41328, |
| | 40016, | 40208, 40240, | 40592, 40624, | 40976, 41008, | | 41360, 41392, |
| | 40048 | 40272, 40304, | 40656, 40688, | 41040, 41072, | | 41424, 41456, |
| | | 40336, 40368, | 40720, 40752, | 41104, 41136, | | 41488, 41520, |
| | | 40400, 40432 | 40784, 40816 | 41168, 41200 | | 41552, 41584 |
| 39921 | 39953, | 40081, 40113, | 40465, 40497, | 40849, 40881, | | 41233, 41265, |
| | 39985, | 40145, 40177, | 40529, 40561, | 40913, 40945, | | 41297, 41329, |
| | 40017, | 40209, 40241, | 40593, 40625, | 40977, 41009, | | 41361, 41393, |
| | 40049 | 40273, 40305, | 40657, 40689, | 41041, 41073, | | 41425, 41457, |
| | | 40337, 40369, | 40721, 40753, | 41105, 41137, | | 41489, 41521, |
| | | 40401, 40433 | 40785, 40817 | 41169, 41201 | | 41553, 41585 |
| 39922 | 39954, | 40082, 40114, | 40466, 40498, | 40850, 40882, | | 41234, 41266, |
| | 39986, | 40146, 40178, | 40530, 40562, | 40914, 40946, | | 41298, 41330, |
| | 40018, | 40210, 40242, | 40594, 40626, | 40978, 41010, | | 41362, 41394, |
| | 40050 | 40274, 40306, | 40658, 40690, | 41042, 41074, | | 41426, 41458, |
| | | 40338, 40370, | 40722, 40754, | 41106, 41138, | | 41490, 41522, |
| | | 40402, 40434 | 40786, 40818 | 41170, 41202 | | 41554, 41586 |
| 39923 | 39955, | 40083, 40115, | 40467, 40499, | 40851, 40883, | | 41235, 41267, |
| | 39987, | 40147, 40179, | 40531, 40563, | 40915, 40947, | | 41299, 41331, |
| | 40019, | 40211, 40243, | 40595, 40627, | 40979, 41011, | | 41363, 41395, |
| | 40051 | 40275, 40307, | 40659, 40691, | 41043, 41075, | | 41427, 41459, |
| | | 40339, 40371, | 40723, 40755, | 41107, 41139, | | 41491, 41523, |
| | | 40403, 40435 | 40787, 40819 | 41171, 41203 | | 41555, 41587 |
| 39924 | 39956, | 40084, 40116, | 40468, 40500, | 40852, 40884, | | 41236, 41268, |
| | 39988, | 40148, 40180, | 40532, 40564, | 40916, 40948, | | 41300, 41332, |
| | 40020, | 40212, 40244, | 40596, 40628, | 40980, 41012, | | 41364, 41396, |
| | 40052 | 40276, 40308, | 40660, 40692, | 41044, 41076, | | 41428, 41460, |
| | | 40340, 40372, | 40724, 40756, | 41108, 41140, | | 41492, 41524, |
| | | 40404, 40436 | 40788, 40820 | 41172, 41204 | | 41556, 41588 |
| 39925 | 39957, | 40085, 40117, | 40469, 40501, | 40853, 40885, | | 41237, 41269, |
| | 39989, | 40149, 40181, | 40533, 40565, | 40917, 40949, | | 41301, 41333, |
| | 40021, | 40213, 40245, | 40597, 40629, | 40981, 41013, | | 41365, 41397, |
| | 40053 | 40277, 40309, | 40661, 40693, | 41045, 41077, | | 41429, 41461, |
| | | 40341, 40373, | 40725, 40757, | 41109, 41141, | | 41493, 41525, |
| | | 40405, 40437 | 40789, 40821 | 41173, 41205 | | 41557, 41589 |
| 39926 | 39958, | 40086, 40118, | 40470, 40502, | 40854, 40886, | | 41238, 41270, |
| | 39990, | 40150, 40182, | 40534, 40566, | 40918, 40950, | | 41302, 41334, |
| | 40022, | 40214, 40246, | 40598, 40630, | 40982, 41014, | | 41366, 41398, |
| | 40054 | 40278, 40310, | 40662, 40694, | 41046, 41078, | | 41430, 41462, |
| | | 40342, 40374, | 40726, 40758, | 41110, 41142, | | 41494, 41526, |
| | | 40406, 40438 | 40790, 40822 | 41174, 41206 | | 41558, 41590 |
| 39927 | 39959, | 40087, 40119, | 40471, 40503, | 40855, 40887, | | 41239, 41271, |
| | 39991, | 40151, 40183, | 40535, 40567, | 40919, 40951, | | 41303, 41335, |
| | 40023, | 40215, 40247, | 40599, 40631, | 40983, 41015, | | 41367, 41399, |
| | 40055 | 40279, 40311, | 40663, 40695, | 41047, 41079, | | 41431, 41463, |
| | | 40343, 40375, | 40727, 40759, | 41111, 41143, | | 41495, 41527, |
| | | 40407, 40439 | 40791, 40823 | 41175, 41207 | | 41559, 41591 |
| 39928 | 39960, | 40088, 40120, | 40472, 40504, | 40856, 40888, | | 41240, 41272, |
| | 39992, | 40152, 40184, | 40536, 40568, | 40920, 40952, | | 41304, 41336, |
| | 40024, | 40216, 40248, | 40600, 40632, | 40984, 41016, | | 41368, 41400, |
| | 40056 | 40280, 40312, | 40664, 40696, | 41048, 41080, | | 41432, 41464, |
| | | 40344, 40376, | 40728, 40760, | 41112, 41144, | | 41496, 41528, |
| | | 40408, 40440 | 40792, 40824 | 41176, 41208 | | 41560, 41592 |
| 39929 | 39961, | 40089, 40121, | 40473, 40505, | 40857, 40889, | | 41241, 41273, |
| | 39993, | 40153, 40185, | 40537, 40569, | 40921, 40953, | | 41305, 41337, |
| | 40025, | 40217, 40249, | 40601, 40633, | 40985, 41017, | | 41369, 41401, |
| | 40057 | 40281, 40313, | 40665, 40697, | 41049, 41081, | | 41433, 41465, |
| | | 40345, 40377, | 40729, 40761, | 41113, 41145, | | 41497, 41529, |
| | | 40409, 40441 | 40793, 40825 | 41177, 41209 | | 41561, 41593 |
| 39930 | 39962, | 40090, 40122, | 40474, 40506, | 40858, 40890, | | 41242, 41274, |
| | 39994, | 40154, 40186, | 40538, 40570, | 40922, 40954, | | 41306, 41338, |
| | 40026, | 40218, 40250, | 40602, 40634, | 40986, 41018, | | 41370, 41402, |
| | 40058 | 40282, 40314, | 40666, 40698, | 41050, 41082, | | 41434, 41466, |
| | | 40346, 40378, | 40730, 40762, | 41114, 41146, | | 41498, 41530, |
| | | 40410, 40442 | 40794, 40826 | 41178, 41210 | | 41562, 41594 |
| 39931 | 39963, | 40091, 40123, | 40475, 40507, | 40859, 40891, | | 41243, 41275, |
| | 39995, | 40155, 40187, | 40539, 40571, | 40923, 40955, | | 41307, 41339, |
| | 40027, | 40219, 40251, | 40603, 40635, | 40987, 41019, | | 41371, 41403, |
| | 40059 | 40283, 40315, | 40667, 40699, | 41051, 41083, | | 41435, 41467, |
| | | 40347, 40379, | 40731, 40763, | 41115, 41147, | | 41499, 41531, |
| | | 40411, 40443 | 40795, 40827 | 41179, 41211 | | 41563, 41595 |
| 39932 | 39964, | 40092, 40124, | 40476, 40508, | 40860, 40892, | | 41244, 41276, |
| | 39996, | 40156, 40188, | 40540, 40572, | 40924, 40956, | | 41308, 41340, |
| | 40028, | 40220, 40252, | 40604, 40636, | 40988, 41020, | | 41372, 41404, |
| | 40060 | 40284, 40316, | 40668, 40700, | 41052, 41084, | | 41436, 41468, |
| | | 40348, 40380, | 40732, 40764, | 41116, 41148, | | 41500, 41532, |
| | | 40412, 40444 | 40796, 40828 | 41180, 41212 | | 41564, 41596 |
| 39933 | 39965, | 40093, 40125, | 40477, 40509, | 40861, 40893, | | 41245, 41277, |
| | 39997, | 40157, 40189, | 40541, 40573, | 40925, 40957, | | 41309, 41341, |
| | 40029, | 40221, 40253, | 40605, 40637, | 40989, 41021, | | 41373, 41405, |
| | 40061 | 40285, 40317, | 40669, 40701, | 41053, 41085, | | 41437, 41469, |
| | | 40349, 40381, | 40733, 40765, | 41117, 41149, | | 41501, 41533, |
| | | 40413, 40445 | 40797, 40829 | 41181, 41213 | | 41565, 41597 |
| 39934 | 39966, | 40094, 40126, | 40478, 40510, | 40862, 40894, | | 41246, 41278, |
| | 39998, | 40158, 40190, | 40542, 40574, | 40926, 40958, | | 41310, 41342, |
| | 40030, | 40222, 40254, | 40606, 40638, | 40990, 41022, | | 41374, 41406, |
| | 40062 | 40286, 40318, | 40670, 40702, | 41054, 41086, | | 41438, 41470, |
| | | 40350, 40382, | 40734, 40766, | 41118, 41150, | | 41502, 41534, |
| | | 40414, 40446 | 40798, 40830 | 41182, 41214 | | 41566, 41598 |
| 39935 | 39967, | 40095, 40127, | 40479, 40511, | 40863, 40895, | | 41247, 41279, |
| | 39999, | 40159, 40191, | 40543, 40575, | 40927, 40959, | | 41311, 41343, |
| | 40031, | 40223, 40255, | 40607, 40639, | 40991, 41023, | | 41375, 41407, |
| | 40063 | 40287, 40319, | 40671, 40703, | 41055, 41087, | | 41439, 41471, |
| | | 40351, 40383, | 40735, 40767, | 41119, 41151, | | 41503, 41535, |
| | | 40415, 40447 | 40799, 40831 | 41183, 41215 | | 41567, 41599 |
| 39936 | 39968, | 40096, 40128, | 40480, 40512, | 40864, 40896, | | 41248, 41280, |
| | 40000, | 40160, 40192, | 40544, 40576, | 40928, 40960, | | 41312, 41344, |
| | 40032, | 40224, 40256, | 40608, 40640, | 40992, 41024, | | 41376, 41408, |
| | 40064 | 40288, 40320, | 40672, 40704, | 41056, 41088, | | 41440, 41472, |
| | | 40352, 40384, | 40736, 40768, | 41120, 41152, | | 41504, 41536, |
| | | 40416, 40448 | 40800, 40832 | 41184, 41216 | | 41568, 41600 |
| 39937 | 39969, | 40097, 40129, | 40481, 40513, | 40865, 40897, | | 41249, 41281, |
| | 40001, | 40161, 40193, | 40545, 40577, | 40929, 40961, | | 41313, 41345, |
| | 40033, | 40225, 40257, | 40609, 40641, | 40993, 41025, | | 41377, 41409, |
| | 40065 | 40289, 40321, | 40673, 40705, | 41057, 41089, | | 41441, 41473, |
| | | 40353, 40385, | 40737, 40769, | 41121, 41153, | | 41505, 41537, |
| | | 40417, 40449 | 40801, 40833 | 41185, 41217 | | 41569, 41601 |
| 39938 | 39970, | 40098, 40130, | 40482, 40514, | 40866, 40898, | | 41250, 41282, |
| | 40002, | 40162, 40194, | 40546, 40578, | 40930, 40962, | | 41314, 41346, |
| | 40034, | 40226, 40258, | 40610, 40642, | 40994, 41026, | | 41378, 41410, |
| | 40066 | 40290, 40322, | 40674, 40706, | 41058, 41090, | | 41442, 41474, |
| | | 40354, 40386, | 40738, 40770, | 41122, 41154, | | 41506, 41538, |
| | | 40418, 40450 | 40802, 40834 | 41186, 41218 | | 41570, 41602 |
| 39939 | 39971, | 40099, 40131, | 40483, 40515, | 40867, 40899, | | 41251, 41283, |
| | 40003, | 40163, 40195, | 40547, 40579, | 40931, 40963, | | 41315, 41347, |
| | 40035, | 40227, 40259, | 40611, 40643, | 40995, 41027, | | 41379, 41411, |
| | 40067 | 40291, 40323, | 40675, 40707, | 41059, 41091, | | 41443, 41475, |
| | | 40355, 40387, | 40739, 40771, | 41123, 41155, | | 41507, 41539, |
| | | 40419, 40451 | 40803, 40835 | 41187, 41219 | | 41571, 41603 |
| 39940 | 39972, | 40100, 40132, | 40484, 40516, | 40868, 40900, | | 41252, 41284, |
| | 40004, | 40164, 40196, | 40548, 40580, | 40932, 40964, | | 41316, 41348, |
| | 40036, | 40228, 40260, | 40612, 40644, | 40996, 41028, | | 41380, 41412, |
| | 40068 | 40292, 40324, | 40676, 40708, | 41060, 41092, | | 41444, 41476, |
| | | 40356, 40388, | 40740, 40772, | 41124, 41156, | | 41508, 41540, |
| | | 40420, 40452 | 40804, 40836 | 41188, 41220 | | 41572, 41604 |

TABLE 3.13
Preferred LASV prefusion-stabilized (GPCmut13) constructs

| | | RNA designs and SEQ ID NOs | | | | |
|---|---|---|---|---|---|---|
| PRT | CDS | a-1 | a-2 | b-4 | i-2 | i-3 |
| 41605 | 41637, | 41765, 41797, | 42149, 42181, | 42533, 42565, | | 42917, 42949, |
| | 41669, | 41829, 41861, | 42213, 42245, | 42597, 42629, | | 42981, 43013, |
| | 41701, | 41893, 41925, | 42277, 42309, | 42661, 42693, | | 43045, 43077, |
| | 41733 | 41957, 41989, | 42341, 42373, | 42725, 42757, | | 43109, 43141, |
| | | 42021, 42053, | 42405, 42437, | 42789, 42821, | | 43173, 43205, |
| | | 42085, 42117 | 42469, 42501 | 42853, 42885 | | 43237, 43269 |
| 41606 | 41638, | 41766, 41798, | 42150, 42182, | 42534, 42566, | | 42918, 42950, |
| | 41670, | 41830, 41862, | 42214, 42246, | 42598, 42630, | | 42982, 43014, |
| | 41702, | 41894, 41926, | 42278, 42310, | 42662, 42694, | | 43046, 43078, |
| | 41734 | 41958, 41990, | 42342, 42374, | 42726, 42758, | | 43110, 43142, |
| | | 42022, 42054, | 42406, 42438, | 42790, 42822, | | 43174, 43206, |
| | | 42086, 42118 | 42470, 42502 | 42854, 42886 | | 43238, 43270 |
| 41607 | 41639, | 41767, 41799, | 42151, 42183, | 42535, 42567, | | 42919, 42951, |
| | 41671, | 41831, 41863, | 42215, 42247, | 42599, 42631, | | 42983, 43015, |
| | 41703, | 41895, 41927, | 42279, 42311, | 42663, 42695, | | 43047, 43079, |
| | 41735 | 41959, 41991, | 42343, 42375, | 42727, 42759, | | 43111, 43143, |
| | | 42023, 42055, | 42407, 42439, | 42791, 42823, | | 43175, 43207, |
| | | 42087, 42119 | 42471, 42503 | 42855, 42887 | | 43239, 43271 |
| 41608 | 41640, | 41768, 41800, | 42152, 42184, | 42536, 42568, | | 42920, 42952, |
| | 41672, | 41832, 41864, | 42216, 42248, | 42600, 42632, | | 42984, 43016, |
| | 41704, | 41896, 41928, | 42280, 42312, | 42664, 42696, | | 43048, 43080, |
| | 41736 | 41960, 41992, | 42344, 42376, | 42728, 42760, | | 43112, 43144, |
| | | 42024, 42056, | 42408, 42440, | 42792, 42824, | | 43176, 43208, |
| | | 42088, 42120 | 42472, 42504 | 42856, 42888 | | 43240, 43272 |
| 41609 | 41641, | 41769, 41801, | 42153, 42185, | 42537, 42569, | | 42921, 42953, |
| | 41673, | 41833, 41865, | 42217, 42249, | 42601, 42633, | | 42985, 43017, |
| | 41705, | 41897, 41929, | 42281, 42313, | 42665, 42697, | | 43049, 43081, |
| | 41737 | 41961, 41993, | 42345, 42377, | 42729, 42761, | | 43113, 43145, |
| | | 42025, 42057, | 42409, 42441, | 42793, 42825, | | 43177, 43209, |
| | | 42089, 42121 | 42473, 42505 | 42857, 42889 | | 43241, 43273 |
| 41610 | 41642, | 41770, 41802, | 42154, 42186, | 42538, 42570, | | 42922, 42954, |
| | 41674, | 41834, 41866, | 42218, 42250, | 42602, 42634, | | 42986, 43018, |
| | 41706, | 41898, 41930, | 42282, 42314, | 42666, 42698, | | 43050, 43082, |
| | 41738 | 41962, 41994, | 42346, 42378, | 42730, 42762, | | 43114, 43146, |
| | | 42026, 42058, | 42410, 42442, | 42794, 42826, | | 43178, 43210, |
| | | 42090, 42122 | 42474, 42506 | 42858, 42890 | | 43242, 43274 |
| 41611 | 41643, | 41771, 41803, | 42155, 42187, | 42539, 42571, | | 42923, 42955, |
| | 41675, | 41835, 41867, | 42219, 42251, | 42603, 42635, | | 42987, 43019, |
| | 41707, | 41899, 41931, | 42283, 42315, | 42667, 42699, | | 43051, 43083, |
| | 41739 | 41963, 41995, | 42347, 42379, | 42731, 42763, | | 43115, 43147, |
| | | 42027, 42059, | 42411, 42443, | 42795, 42827, | | 43179, 43211, |
| | | 42091, 42123 | 42475, 42507 | 42859, 42891 | | 43243, 43275 |
| 41612 | 41644, | 41772, 41804, | 42156, 42188, | 42540, 42572, | | 42924, 42956, |
| | 41676, | 41836, 41868, | 42220, 42252, | 42604, 42636, | | 42988, 43020, |
| | 41708, | 41900, 41932, | 42284, 42316, | 42668, 42700, | | 43052, 43084, |
| | 41740 | 41964, 41996, | 42348, 42380, | 42732, 42764, | | 43116, 43148, |
| | | 42028, 42060, | 42412, 42444, | 42796, 42828, | | 43180, 43212, |
| | | 42092, 42124 | 42476, 42508 | 42860, 42892 | | 43244, 43276 |
| 41613 | 41645, | 41773, 41805, | 42157, 42189, | 42541, 42573, | | 42925, 42957, |
| | 41677, | 41837, 41869, | 42221, 42253, | 42605, 42637, | | 42989, 43021, |
| | 41709, | 41901, 41933, | 42285, 42317, | 42669, 42701, | | 43053, 43085, |
| | 41741 | 41965, 41997, | 42349, 42381, | 42733, 42765, | | 43117, 43149, |
| | | 42029, 42061, | 42413, 42445, | 42797, 42829, | | 43181, 43213, |
| | | 42093, 42125 | 42477, 42509 | 42861, 42893 | | 43245, 43277 |
| 41614 | 41646, | 41774, 41806, | 42158, 42190, | 42542, 42574, | | 42926, 42958, |
| | 41678, | 41838, 41870, | 42222, 42254, | 42606, 42638, | | 42990, 43022, |
| | 41710, | 41902, 41934, | 42286, 42318, | 42670, 42702, | | 43054, 43086, |
| | 41742 | 41966, 41998, | 42350, 42382, | 42734, 42766, | | 43118, 43150, |
| | | 42030, 42062, | 42414, 42446, | 42798, 42830, | | 43182, 43214, |
| | | 42094, 42126 | 42478, 42510 | 42862, 42894 | | 43246, 43278 |
| 41615 | 41647, | 41775, 41807, | 42159, 42191, | 42543, 42575, | | 42927, 42959, |
| | 41679, | 41839, 41871, | 42223, 42255, | 42607, 42639, | | 42991, 43023, |
| | 41711, | 41903, 41935, | 42287, 42319, | 42671, 42703, | | 43055, 43087, |
| | 41743 | 41967, 41999, | 42351, 42383, | 42735, 42767, | | 43119, 43151, |
| | | 42031, 42063, | 42415, 42447, | 42799, 42831, | | 43183, 43215, |
| | | 42095, 42127 | 42479, 42511 | 42863, 42895 | | 43247, 43279 |
| 41616 | 41648, | 41776, 41808, | 42160, 42192, | 42544, 42576, | | 42928, 42960, |
| | 41680, | 41840, 41872, | 42224, 42256, | 42608, 42640, | | 42992, 43024, |
| | 41712, | 41904, 41936, | 42288, 42320, | 42672, 42704, | | 43056, 43088, |
| | 41744 | 41968, 42000, | 42352, 42384, | 42736, 42768, | | 43120, 43152, |
| | | 42032, 42064, | 42416, 42448, | 42800, 42832, | | 43184, 43216, |
| | | 42096, 42128 | 42480, 42512 | 42864, 42896 | | 43248, 43280 |
| 41617 | 41649, | 41777, 41809, | 42161, 42193, | 42545, 42577, | | 42929, 42961, |
| | 41681, | 41841, 41873, | 42225, 42257, | 42609, 42641, | | 42993, 43025, |
| | 41713, | 41905, 41937, | 42289, 42321, | 42673, 42705, | | 43057, 43089, |
| | 41745 | 41969, 42001, | 42353, 42385, | 42737, 42769, | | 43121, 43153, |
| | | 42033, 42065, | 42417, 42449, | 42801, 42833, | | 43185, 43217, |
| | | 42097, 42129 | 42481, 42513 | 42865, 42897 | | 43249, 43281 |
| 41618 | 41650, | 41778, 41810, | 42162, 42194, | 42546, 42578, | | 42930, 42962, |
| | 41682, | 41842, 41874, | 42226, 42258, | 42610, 42642, | | 42994, 43026, |
| | 41714, | 41906, 41938, | 42290, 42322, | 42674, 42706, | | 43058, 43090, |
| | 41746 | 41970, 42002, | 42354, 42386, | 42738, 42770, | | 43122, 43154, |
| | | 42034, 42066, | 42418, 42450, | 42802, 42834, | | 43186, 43218, |
| | | 42098, 42130 | 42482, 42514 | 42866, 42898 | | 43250, 43282 |
| 41619 | 41651, | 41779, 41811, | 42163, 42195, | 42547, 42579, | | 42931, 42963, |
| | 41683, | 41843, 41875, | 42227, 42259, | 42611, 42643, | | 42995, 43027, |
| | 41715, | 41907, 41939, | 42291, 42323, | 42675, 42707, | | 43059, 43091, |
| | 41747 | 41971, 42003, | 42355, 42387, | 42739, 42771, | | 43123, 43155, |
| | | 42035, 42067, | 42419, 42451, | 42803, 42835, | | 43187, 43219, |
| | | 42099, 42131 | 42483, 42515 | 42867, 42899 | | 43251, 43283 |
| 41620 | 41652, | 41780, 41812, | 42164, 42196, | 42548, 42580, | | 42932, 42964, |
| | 41684, | 41844, 41876, | 42228, 42260, | 42612, 42644, | | 42996, 43028, |
| | 41716, | 41908, 41940, | 42292, 42324, | 42676, 42708, | | 43060, 43092, |
| | 41748 | 41972, 42004, | 42356, 42388, | 42740, 42772, | | 43124, 43156, |
| | | 42036, 42068, | 42420, 42452, | 42804, 42836, | | 43188, 43220, |
| | | 42100, 42132 | 42484, 42516 | 42868, 42900 | | 43252, 43284 |
| 41621 | 41653, | 41781, 41813, | 42165, 42197, | 42549, 42581, | | 42933, 42965, |
| | 41685, | 41845, 41877, | 42229, 42261, | 42613, 42645, | | 42997, 43029, |
| | 41717, | 41909, 41941, | 42293, 42325, | 42677, 42709, | | 43061, 43093, |
| | 41749 | 41973, 42005, | 42357, 42389, | 42741, 42773, | | 43125, 43157, |
| | | 42037, 42069, | 42421, 42453, | 42805, 42837, | | 43189, 43221, |
| | | 42101, 42133 | 42485, 42517 | 42869, 42901 | | 43253, 43285 |
| 41622 | 41654, | 41782, 41814, | 42166, 42198, | 42550, 42582, | | 42934, 42966, |
| | 41686, | 41846, 41878, | 42230, 42262, | 42614, 42646, | | 42998, 43030, |
| | 41718, | 41910, 41942, | 42294, 42326, | 42678, 42710, | | 43062, 43094, |
| | 41750 | 41974, 42006, | 42358, 42390, | 42742, 42774, | | 43126, 43158, |
| | | 42038, 42070, | 42422, 42454, | 42806, 42838, | | 43190, 43222, |
| | | 42102, 42134 | 42486, 42518 | 42870, 42902 | | 43254, 43286 |
| 41623 | 41655, | 41783, 41815, | 42167, 42199, | 42551, 42583, | | 42935, 42967, |
| | 41687, | 41847, 41879, | 42231, 42263, | 42615, 42647, | | 42999, 43031, |
| | 41719, | 41911, 41943, | 42295, 42327, | 42679, 42711, | | 43063, 43095, |
| | 41751 | 41975, 42007, | 42359, 42391, | 42743, 42775, | | 43127, 43159, |
| | | 42039, 42071, | 42423, 42455, | 42807, 42839, | | 43191, 43223, |
| | | 42103, 42135 | 42487, 42519 | 42871, 42903 | | 43255, 43287 |
| 41624 | 41656, | 41784, 41816, | 42168, 42200, | 42552, 42584, | | 42936, 42968, |
| | 41688, | 41848, 41880, | 42232, 42264, | 42616, 42648, | | 43000, 43032, |
| | 41720, | 41912, 41944, | 42296, 42328, | 42680, 42712, | | 43064, 43096, |
| | 41752 | 41976, 42008, | 42360, 42392, | 42744, 42776, | | 43128, 43160, |
| | | 42040, 42072, | 42424, 42456, | 42808, 42840, | | 43192, 43224, |
| | | 42104, 42136 | 42488, 42520 | 42872, 42904 | | 43256, 43288 |
| 41625 | 41657, | 41785, 41817, | 42169, 42201, | 42553, 42585, | | 42937, 42969, |
| | 41689, | 41849, 41881, | 42233, 42265, | 42617, 42649, | | 43001, 43033, |
| | 41721, | 41913, 41945, | 42297, 42329, | 42681, 42713, | | 43065, 43097, |
| | 41753 | 41977, 42009, | 42361, 42393, | 42745, 42777, | | 43129, 43161, |
| | | 42041, 42073, | 42425, 42457, | 42809, 42841, | | 43193, 43225, |
| | | 42105, 42137 | 42489, 42521 | 42873, 42905 | | 43257, 43289 |
| 41626 | 41658, | 41786, 41818, | 42170, 42202, | 42554, 42586, | | 42938, 42970, |
| | 41690, | 41850, 41882, | 42234, 42266, | 42618, 42650, | | 43002, 43034, |
| | 41722, | 41914, 41946, | 42298, 42330, | 42682, 42714, | | 43066, 43098, |
| | 41754 | 41978, 42010, | 42362, 42394, | 42746, 42778, | | 43130, 43162, |
| | | 42042, 42074, | 42426, 42458, | 42810, 42842, | | 43194, 43226, |
| | | 42106, 42138 | 42490, 42522 | 42874, 42906 | | 43258, 43290 |
| 41627 | 41659, | 41787, 41819, | 42171, 42203, | 42555, 42587, | | 42939, 42971, |
| | 41691, | 41851, 41883, | 42235, 42267, | 42619, 42651, | | 43003, 43035, |
| | 41723, | 41915, 41947, | 42299, 42331, | 42683, 42715, | | 43067, 43099, |
| | 41755 | 41979, 42011, | 42363, 42395, | 42747, 42779, | | 43131, 43163, |
| | | 42043, 42075, | 42427, 42459, | 42811, 42843, | | 43195, 43227, |
| | | 42107, 42139 | 42491, 42523 | 42875, 42907 | | 43259, 43291 |
| 41628 | 41660, | 41788, 41820, | 42172, 42204, | 42556, 42588, | | 42940, 42972, |
| | 41692, | 41852, 41884, | 42236, 42268, | 42620, 42652, | | 43004, 43036, |
| | 41724, | 41916, 41948, | 42300, 42332, | 42684, 42716, | | 43068, 43100, |
| | 41756 | 41980, 42012, | 42364, 42396, | 42748, 42780, | | 43132, 43164, |
| | | 42044, 42076, | 42428, 42460, | 42812, 42844, | | 43196, 43228, |
| | | 42108, 42140 | 42492, 42524 | 42876, 42908 | | 43260, 43292 |
| 41629 | 41661, | 41789, 41821, | 42173, 42205, | 42557, 42589, | | 42941, 42973, |
| | 41693, | 41853, 41885, | 42237, 42269, | 42621, 42653, | | 43005, 43037, |
| | 41725, | 41917, 41949, | 42301, 42333, | 42685, 42717, | | 43069, 43101, |
| | 41757 | 41981, 42013, | 42365, 42397, | 42749, 42781, | | 43133, 43165, |
| | | 42045, 42077, | 42429, 42461, | 42813, 42845, | | 43197, 43229, |
| | | 42109, 42141 | 42493, 42525 | 42877, 42909 | | 43261, 43293 |
| 41630 | 41662, | 41790, 41822, | 42174, 42206, | 42558, 42590, | | 42942, 42974, |
| | 41694, | 41854, 41886, | 42238, 42270, | 42622, 42654, | | 43006, 43038, |
| | 41726, | 41918, 41950, | 42302, 42334, | 42686, 42718, | | 43070, 43102, |
| | 41758 | 41982, 42014, | 42366, 42398, | 42750, 42782, | | 43134, 43166, |
| | | 42046, 42078, | 42430, 42462, | 42814, 42846, | | 43198, 43230, |
| | | 42110, 42142 | 42494, 42526 | 42878, 42910 | | 43262, 43294 |
| 41631 | 41663, | 41791, 41823, | 42175, 42207, | 42559, 42591, | | 42943, 42975, |
| | 41695, | 41855, 41887, | 42239, 42271, | 42623, 42655, | | 43007, 43039, |
| | 41727, | 41919, 41951, | 42303, 42335, | 42687, 42719, | | 43071, 43103, |
| | 41759 | 41983, 42015, | 42367, 42399, | 42751, 42783, | | 43135, 43167, |
| | | 42047, 42079, | 42431, 42463, | 42815, 42847, | | 43199, 43231, |
| | | 42111, 42143 | 42495, 42527 | 42879, 42911 | | 43263, 43295 |
| 41632 | 41664, | 41792, 41824, | 42176, 42208, | 42560, 42592, | | 42944, 42976, |
| | 41696, | 41856, 41888, | 42240, 42272, | 42624, 42656, | | 43008, 43040, |
| | 41728, | 41920, 41952, | 42304, 42336, | 42688, 42720, | | 43072, 43104, |
| | 41760 | 41984, 42016, | 42368, 42400, | 42752, 42784, | | 43136, 43168, |
| | | 42048, 42080, | 42432, 42464, | 42816, 42848, | | 43200, 43232, |
| | | 42112, 42144 | 42496, 42528 | 42880, 42912 | | 43264, 43296 |
| 41633 | 41665, | 41793, 41825, | 42177, 42209, | 42561, 42593, | | 42945, 42977, |
| | 41697, | 41857, 41889, | 42241, 42273, | 42625, 42657, | | 43009, 43041, |
| | 41729, | 41921, 41953, | 42305, 42337, | 42689, 42721, | | 43073, 43105, |
| | 41761 | 41985, 42017, | 42369, 42401, | 42753, 42785, | | 43137, 43169, |
| | | 42049, 42081, | 42433, 42465, | 42817, 42849, | | 43201, 43233, |
| | | 42113, 42145 | 42497, 42529 | 42881, 42913 | | 43265, 43297 |
| 41634 | 41666, | 41794, 41826, | 42178, 42210, | 42562, 42594, | | 42946, 42978, |
| | 41698, | 41858, 41890, | 42242, 42274, | 42626, 42658, | | 43010, 43042, |
| | 41730, | 41922, 41954, | 42306, 42338, | 42690, 42722, | | 43074, 43106, |
| | 41762 | 41986, 42018, | 42370, 42402, | 42754, 42786, | | 43138, 43170, |
| | | 42050, 42082, | 42434, 42466, | 42818, 42850, | | 43202, 43234, |
| | | 42114, 42146 | 42498, 42530 | 42882, 42914 | | 43266, 43298 |
| 41635 | 41667, | 41795, 41827, | 42179, 42211, | 42563, 42595, | | 42947, 42979, |
| | 41699, | 41859, 41891, | 42243, 42275, | 42627, 42659, | | 43011, 43043, |
| | 41731, | 41923, 41955, | 42307, 42339, | 42691, 42723, | | 43075, 43107, |
| | 41763 | 41987, 42019, | 42371, 42403, | 42755, 42787, | | 43139, 43171, |
| | | 42051, 42083, | 42435, 42467, | 42819, 42851, | | 43203, 43235, |
| | | 42115, 42147 | 42499, 42531 | 42883, 42915 | | 43267, 43299 |
| 41636 | 41668, | 41796, 41828, | 42180, 42212, | 42564, 42596, | | 42948, 42980, |
| | 41700, | 41860, 41892, | 42244, 42276, | 42628, 42660, | | 43012, 43044, |
| | 41732, | 41924, 41956, | 42308, 42340, | 42692, 42724, | | 43076, 43108, |
| | 41764 | 41988, 42020, | 42372, 42404, | 42756, 42788, | | 43140, 43172, |
| | | 42052, 42084, | 42436, 42468, | 42820, 42852, | | 43204, 43236, |
| | | 42116, 42148 | 42500, 42532 | 42884, 42916 | | 43268, 43300 |

TABLE 4
Preferred LASV Nucleoprotein (NP) constructs

| | | RNA designs and SEQ ID NOs | | | | |
|---|---|---|---|---|---|---|
| PRT | CDS | a-1 | a-2 | b-4 | i-2 | i-3 |
| 7547 | 7798, | 9806, 9840, | 10078, 10112, | 10350, 10384, | 10622, | 10894, 10928, |
| | 8049, | 9874, 9908, | 10146, 10180, | 10418, 10452, | 10656, | 10962, 10996, |
| | 8300, | 9942, 9976, | 10214, 10248, | 10486, 10520, | 10690, | 11030, 11064, |
| | 8551, | 10010, 10044, | 10282, 10316, | 10554, 10588, | 10724, | 11098, 11132, |
| | 8802, | 16744, 16778, | 17050, 17084, | 17356, 17390, | 10758, | 17662, 17696, |
| | 9053, | 16812, 16846, | 17118, 17152, | 17424, 17458, | 10792, | 17730, 17764, |
| | 9304, | 16880, 16914, | 17186, 17220, | 17492, 17526, | 10826, | 17798, 17832, |
| | 9555 | 16948, 16982, | 17254, 17288, | 17560, 17594, | 10860 | 17866, 17900, |
| | | 17016 | 17322 | 17628 | | 17934 |
| 7548 | 7799, | 9807, 9841, | 10079, 10113, | 10351, 10385, | 10623, | 10895, 10929, |
| | 8050, | 9875, 9909, | 10147, 10181, | 10419, 10453, | 10657, | 10963, 10997, |
| | 8301, | 9943, 9977, | 10215, 10249, | 10487, 10521, | 10691, | 11031, 11065, |
| | 8552, | 10011, 10045, | 10283, 10317, | 10555, 10589, | 10725, | 11099, 11133, |
| | 8803, | 16745, 16779, | 17051, 17085, | 17357, 17391, | 10759, | 17663, 17697, |
| | 9054, | 16813, 16847, | 17119, 17153, | 17425, 17459, | 10793, | 17731, 17765, |
| | 9305, | 16881, 16915, | 17187, 17221, | 17493, 17527, | 10827, | 17799, 17833, |
| | 9556 | 16949, 16983, | 17255, 17289, | 17561, 17595, | 10861 | 17867, 17901, |
| | | 17017 | 17323 | 17629 | | 17935 |
| 7549 | 7800, | 9808, 9842, | 10080, 10114, | 10352, 10386, | 10624, | 10896, 10930, |
| | 8051, | 9876, 9910, | 10148, 10182, | 10420, 10454, | 10658, | 10964, 10998, |
| | 8302, | 9944, 9978, | 10216, 10250, | 10488, 10522, | 10692, | 11032, 11066, |
| | 8553, | 10012, 10046, | 10284, 10318, | 10556, 10590, | 10726, | 11100, 11134, |
| | 8804, | 16746, 16780, | 17052, 17086, | 17358, 17392, | 10760, | 17664, 17698, |
| | 9055, | 16814, 16848, | 17120, 17154, | 17426, 17460, | 10794, | 17732, 17766, |
| | 9306, | 16882, 16916, | 17188, 17222, | 17494, 17528, | 10828, | 17800, 17834, |
| | 9557 | 16950, 16984, | 17256, 17290, | 17562, 17596, | 10862 | 17868, 17902, |
| | | 17018 | 17324 | 17630 | | 17936 |
| 7550 | 7801, | 9809, 9843, | 10081, 10115, | 10353, 10387, | 10625, | 10897, 10931, |
| | 8052, | 9877, 9911, | 10149, 10183, | 10421, 10455, | 10659, | 10965, 10999, |
| | 8303, | 9945, 9979, | 10217, 10251, | 10489, 10523, | 10693, | 11033, 11067, |
| | 8554, | 10013, 10047, | 10285, 10319, | 10557, 10591, | 10727, | 11101, 11135, |
| | 8805, | 16747, 16781, | 17053, 17087, | 17359, 17393, | 10761, | 17665, 17699, |
| | 9056, | 16815, 16849, | 17121, 17155, | 17427, 17461, | 10795, | 17733, 17767, |
| | 9307, | 16883, 16917, | 17189, 17223, | 17495, 17529, | 10829, | 17801, 17835, |
| | 9558 | 16951, 16985, | 17257, 17291, | 17563, 17597, | 10863 | 17869, 17903, |
| | | 17019 | 17325 | 17631 | | 17937 |
| 7551 | 7802, | 9810, 9844, | 10082, 10116, | 10354, 10388, | 10626, | 10898, 10932, |
| | 8053, | 9878, 9912, | 10150, 10184, | 10422, 10456, | 10660, | 10966, 11000, |
| | 8304, | 9946, 9980, | 10218, 10252, | 10490, 10524, | 10694, | 11034, 11068, |
| | 8555, | 10014, 10048, | 10286, 10320, | 10558, 10592, | 10728, | 11102, 11136, |
| | 8806, | 16748, 16782, | 17054, 17088, | 17360, 17394, | 10762, | 17666, 17700, |
| | 9057, | 16816, 16850, | 17122, 17156, | 17428, 17462, | 10796, | 17734, 17768, |
| | 9308, | 16884, 16918, | 17190, 17224, | 17496, 17530, | 10830, | 17802, 17836, |
| | 9559 | 16952, 16986, | 17258, 17292, | 17564, 17598, | 10864 | 17870, 17904, |
| | | 17020 | 17326 | 17632 | | 17938 |
| 7552 | 7803, | 9811, 9845, | 10083, 10117, | 10355, 10389, | 10627, | 10899, 10933, |
| | 8054, | 9879, 9913, | 10151, 10185, | 10423, 10457, | 10661, | 10967, 11001, |
| | 8305, | 9947, 9981, | 10219, 10253, | 10491, 10525, | 10695, | 11035, 11069, |
| | 8556, | 10015, 10049, | 10287, 10321, | 10559, 10593, | 10729, | 11103, 11137, |
| | 8807, | 16749, 16783, | 17055, 17089, | 17361, 17395, | 10763, | 17667, 17701, |
| | 9058, | 16817, 16851, | 17123, 17157, | 17429, 17463, | 10797, | 17735, 17769, |
| | 9309, | 16885, 16919, | 17191, 17225, | 17497, 17531, | 10831, | 17803, 17837, |
| | 9560 | 16953, 16987, | 17259, 17293, | 17565, 17599, | 10865 | 17871, 17905, |
| | | 17021 | 17327 | 17633 | | 17939 |
| 7553 | 7804, | 9812, 9846, | 10084, 10118, | 10356, 10390, | 10628, | 10900, 10934, |
| | 8055, | 9880, 9914, | 10152, 10186, | 10424, 10458, | 10662, | 10968, 11002, |
| | 8306, | 9948, 9982, | 10220, 10254, | 10492, 10526, | 10696, | 11036, 11070, |
| | 8557, | 10016, 10050, | 10288, 10322, | 10560, 10594, | 10730, | 11104, 11138, |
| | 8808, | 16750, 16784, | 17056, 17090, | 17362, 17396, | 10764, | 17668, 17702, |
| | 9059, | 16818, 16852, | 17124, 17158, | 17430, 17464, | 10798, | 17736, 17770, |
| | 9310, | 16886, 16920, | 17192, 17226, | 17498, 17532, | 10832, | 17804, 17838, |
| | 9561 | 16954, 16988, | 17260, 17294, | 17566, 17600, | 10866 | 17872, 17906, |
| | | 17022 | 17328 | 17634 | | 17940 |
| 7554 | 7805, | 9813, 9847, | 10085, 10119, | 10357, 10391, | 10629, | 10901, 10935, |
| | 8056, | 9881, 9915, | 10153, 10187, | 10425, 10459, | 10663, | 10969, 11003, |
| | 8307, | 9949, 9983, | 10221, 10255, | 10493, 10527, | 10697, | 11037, 11071, |
| | 8558, | 10017, 10051, | 10289, 10323, | 10561, 10595, | 10731, | 11105, 11139, |
| | 8809, | 16751, 16785, | 17057, 17091, | 17363, 17397, | 10765, | 17669, 17703, |
| | 9060, | 16819, 16853, | 17125, 17159, | 17431, 17465, | 10799, | 17737, 17771, |
| | 9311, | 16887, 16921, | 17193, 17227, | 17499, 17533, | 10833, | 17805, 17839, |
| | 9562 | 16955, 16989, | 17261, 17295, | 17567, 17601, | 10867 | 17873, 17907, |
| | | 17023 | 17329 | 17635 | | 17941 |
| 7555 | 7806, | 9814, 9848, | 10086, 10120, | 10358, 10392, | 10630, | 10902, 10936, |
| | 8057, | 9882, 9916, | 10154, 10188, | 10426, 10460, | 10664, | 10970, 11004, |
| | 8308, | 9950, 9984, | 10222, 10256, | 10494, 10528, | 10698, | 11038, 11072, |
| | 8559, | 10018, 10052, | 10290, 10324, | 10562, 10596, | 10732, | 11106, 11140, |
| | 8810, | 16752, 16786, | 17058, 17092, | 17364, 17398, | 10766, | 17670, 17704, |
| | 9061, | 16820, 16854, | 17126, 17160, | 17432, 17466, | 10800, | 17738, 17772, |
| | 9312, | 16888, 16922, | 17194, 17228, | 17500, 17534, | 10834, | 17806, 17840, |
| | 9563 | 16956, 16990, | 17262, 17296, | 17568, 17602, | 10868 | 17874, 17908, |
| | | 17024 | 17330 | 17636 | | 17942 |
| 7573 | 7824, | 9815, 9849, | 10087, 10121, | 10359, 10393, | 10631, | 10903, 10937, |
| | 8075, | 9883, 9917, | 10155, 10189, | 10427, 10461, | 10665, | 10971, 11005, |
| | 8326, | 9951, 9985, | 10223, 10257, | 10495, 10529, | 10699, | 11039, 11073, |
| | 8577, | 10019, 10053, | 10291, 10325, | 10563, 10597, | 10733, | 11107, 11141, |
| | 8828, | 16753, 16787, | 17059, 17093, | 17365, 17399, | 10767, | 17671, 17705, |
| | 9079, | 16821, 16855, | 17127, 17161, | 17433, 17467, | 10801, | 17739, 17773, |
| | 9330, | 16889, 16923, | 17195, 17229, | 17501, 17535, | 10835, | 17807, 17841, |
| | 9581 | 16957, 16991, | 17263, 17297, | 17569, 17603, | 10869 | 17875, 17909, |
| | | 17025 | 17331 | 17637 | | 17943 |
| 7593 | 7844, | 9816, 9850, | 10088, 10122, | 10360, 10394, | 10632, | 10904, 10938, |
| | 8095, | 9884, 9918, | 10156, 10190, | 10428, 10462, | 10666, | 10972, 11006, |
| | 8346, | 9952, 9986, | 10224, 10258, | 10496, 10530, | 10700, | 11040, 11074, |
| | 8597, | 10020, 10054, | 10292, 10326, | 10564, 10598, | 10734, | 11108, 11142, |
| | 8848, | 16754, 16788, | 17060, 17094, | 17366, 17400, | 10768, | 17672, 17706, |
| | 9099, | 16822, 16856, | 17128, 17162, | 17434, 17468, | 10802, | 17740, 17774, |
| | 9350, | 16890, 16924, | 17196, 17230, | 17502, 17536, | 10836, | 17808, 17842, |
| | 9601 | 16958, 16992, | 17264, 17298, | 17570, 17604, | 10870 | 17876, 17910, |
| | | 17026 | 17332 | 17638 | | 17944 |
| 7602 | 7853, | 9817, 9851, | 10089, 10123, | 10361, 10395, | 10633, | 10905, 10939, |
| | 8104, | 9885, 9919, | 10157, 10191, | 10429, 10463, | 10667, | 10973, 11007, |
| | 8355, | 9953, 9987, | 10225, 10259, | 10497, 10531, | 10701, | 11041, 11075, |
| | 8606, | 10021, 10055, | 10293, 10327, | 10565, 10599, | 10735, | 11109, 11143, |
| | 8857, | 16755, 16789, | 17061, 17095, | 17367, 17401, | 10769, | 17673, 17707, |
| | 9108, | 16823, 16857, | 17129, 17163, | 17435, 17469, | 10803, | 17741, 17775, |
| | 9359, | 16891, 16925, | 17197, 17231, | 17503, 17537, | 10837, | 17809, 17843, |
| | 9610 | 16959, 16993, | 17265, 17299, | 17571, 17605, | 10871 | 17877, 17911, |
| | | 17027 | 17333 | 17639 | | 17945 |
| 7603 | 7854, | 9818, 9852, | 10090, 10124, | 10362, 10396, | 10634, | 10906, 10940, |
| | 8105, | 9886, 9920, | 10158, 10192, | 10430, 10464, | 10668, | 10974, 11008, |
| | 8356, | 9954, 9988, | 10226, 10260, | 10498, 10532, | 10702, | 11042, 11076, |
| | 8607, | 10022, 10056, | 10294, 10328, | 10566, 10600, | 10736, | 11110, 11144, |
| | 8858, | 16756, 16790, | 17062, 17096, | 17368, 17402, | 10770, | 17674, 17708, |
| | 9109, | 16824, 16858, | 17130, 17164, | 17436, 17470, | 10804, | 17742, 17776, |
| | 9360, | 16892, 16926, | 17198, 17232, | 17504, 17538, | 10838, | 17810, 17844, |
| | 9611 | 16960, 16994, | 17266, 17300, | 17572, 17606, | 10872 | 17878, 17912, |
| | | 17028 | 17334 | 17640 | | 17946 |
| 7645 | 7896, | 9819, 9853, | 10091, 10125, | 10363, 10397, | 10635, | 10907, 10941, |
| | 8147, | 9887, 9921, | 10159, 10193, | 10431, 10465, | 10669, | 10975, 11009, |
| | 8398, | 9955, 9989, | 10227, 10261, | 10499, 10533, | 10703, | 11043, 11077, |
| | 8649, | 10023, 10057, | 10295, 10329, | 10567, 10601, | 10737, | 11111, 11145, |
| | 8900, | 16757, 16791, | 17063, 17097, | 17369, 17403, | 10771, | 17675, 17709, |
| | 9151, | 16825, 16859, | 17131, 17165, | 17437, 17471, | 10805, | 17743, 17777, |
| | 9402, | 16893, 16927, | 17199, 17233, | 17505, 17539, | 10839, | 17811, 17845, |
| | 9653 | 16961, 16995, | 17267, 17301, | 17573, 17607, | 10873 | 17879, 17913, |
| | | 17029 | 17335 | 17641 | | 17947 |
| 7650 | 7901, | 9820, 9854, | 10092, 10126, | 10364, 10398, | 10636, | 10908, 10942, |
| | 8152, | 9888, 9922, | 10160, 10194, | 10432, 10466, | 10670, | 10976, 11010, |
| | 8403, | 9956, 9990, | 10228, 10262, | 10500, 10534, | 10704, | 11044, 11078, |
| | 8654, | 10024, 10058, | 10296, 10330, | 10568, 10602, | 10738, | 11112, 11146, |
| | 8905, | 16758, 16792, | 17064, 17098, | 17370, 17404, | 10772, | 17676, 17710, |
| | 9156, | 16826, 16860, | 17132, 17166, | 17438, 17472, | 10806, | 17744, 17778, |
| | 9407, | 16894, 16928, | 17200, 17234, | 17506, 17540, | 10840, | 17812, 17846, |
| | 9658 | 16962, 16996, | 17268, 17302, | 17574, 17608, | 10874 | 17880, 17914, |
| | | 17030 | 17336 | 17642 | | 17948 |
| 7651 | 7902, | 9821, 9855, | 10093, 10127, | 10365, 10399, | 10637, | 10909, 10943, |
| | 8153, | 9889, 9923, | 10161, 10195, | 10433, 10467, | 10671, | 10977, 11011, |
| | 8404, | 9957, 9991, | 10229, 10263, | 10501, 10535, | 10705, | 11045, 11079, |
| | 8655, | 10025, 10059, | 10297, 10331, | 10569, 10603, | 10739, | 11113, 11147, |
| | 8906, | 16759, 16793, | 17065, 17099, | 17371, 17405, | 10773, | 17677, 17711, |
| | 9157, | 16827, 16861, | 17133, 17167, | 17439, 17473, | 10807, | 17745, 17779, |
| | 9408, | 16895, 16929, | 17201, 17235, | 17507, 17541, | 10841, | 17813, 17847, |
| | 9659 | 16963, 16997, | 17269, 17303, | 17575, 17609, | 10875 | 17881, 17915, |
| | | 17031 | 17337 | 17643 | | 17949 |
| 7658 | 7909, | 9822, 9856, | 10094, 10128, | 10366, 10400, | 10638, | 10910, 10944, |
| | 8160, | 9890, 9924, | 10162, 10196, | 10434, 10468, | 10672, | 10978, 11012, |
| | 8411, | 9958, 9992, | 10230, 10264, | 10502, 10536, | 10706, | 11046, 11080, |
| | 8662, | 10026, 10060, | 10298, 10332, | 10570, 10604, | 10740, | 11114, 11148, |
| | 8913, | 16760, 16794, | 17066, 17100, | 17372, 17406, | 10774, | 17678, 17712, |
| | 9164, | 16828, 16862, | 17134, 17168, | 17440, 17474, | 10808, | 17746, 17780, |
| | 9415, | 16896, 16930, | 17202, 17236, | 17508, 17542, | 10842, | 17814, 17848, |
| | 9666 | 16964, 16998, | 17270, 17304, | 17576, 17610, | 10876 | 17882, 17916, |
| | | 17032 | 17338 | 17644 | | 17950 |
| 7662 | 7913, | 9823, 9857, | 10095, 10129, | 10367, 10401, | 10639, | 10911, 10945, |
| | 8164, | 9891, 9925, | 10163, 10197, | 10435, 10469, | 10673, | 10979, 11013, |
| | 8415, | 9959, 9993, | 10231, 10265, | 10503, 10537, | 10707, | 11047, 11081, |
| | 8666, | 10027, 10061, | 10299, 10333, | 10571, 10605, | 10741, | 11115, 11149, |
| | 8917, | 16761, 16795, | 17067, 17101, | 17373, 17407, | 10775, | 17679, 17713, |
| | 9168, | 16829, 16863, | 17135, 17169, | 17441, 17475, | 10809, | 17747, 17781, |
| | 9419, | 16897, 16931, | 17203, 17237, | 17509, 17543, | 10843, | 17815, 17849, |
| | 9670 | 16965, 16999, | 17271, 17305, | 17577, 17611, | 10877 | 17883, 17917, |
| | | 17033 | 17339 | 17645 | | 17951 |
| 7666 | 7917, | 9824, 9858, | 10096, 10130, | 10368, 10402, | 10640, | 10912, 10946, |
| | 8168, | 9892, 9926, | 10164, 10198, | 10436, 10470, | 10674, | 10980, 11014, |
| | 8419, | 9960, 9994, | 10232, 10266, | 10504, 10538, | 10708, | 11048, 11082, |
| | 8670, | 10028, 10062, | 10300, 10334, | 10572, 10606, | 10742, | 11116, 11150, |
| | 8921, | 16762, 16796, | 17068, 17102, | 17374, 17408, | 10776, | 17680, 17714, |
| | 9172, | 16830, 16864, | 17136, 17170, | 17442, 17476, | 10810, | 17748, 17782, |
| | 9423, | 16898, 16932, | 17204, 17238, | 17510, 17544, | 10844, | 17816, 17850, |
| | 9674 | 16966, 17000, | 17272, 17306, | 17578, 17612, | 10878 | 17884, 17918, |
| | | 17034 | 17340 | 17646 | | 17952 |
| 7671 | 7922, | 9825, 9859, | 10097, 10131, | 10369, 10403, | 10641, | 10913, 10947, |
| | 8173, | 9893, 9927, | 10165, 10199, | 10437, 10471, | 10675, | 10981, 11015, |
| | 8424, | 9961, 9995, | 10233, 10267, | 10505, 10539, | 10709, | 11049, 11083, |
| | 8675, | 10029, 10063, | 10301, 10335, | 10573, 10607, | 10743, | 11117, 11151, |
| | 8926, | 16763, 16797, | 17069, 17103, | 17375, 17409, | 10777, | 17681, 17715, |
| | 9177, | 16831, 16865, | 17137, 17171, | 17443, 17477, | 10811, | 17749, 17783, |
| | 9428, | 16899, 16933, | 17205, 17239, | 17511, 17545, | 10845, | 17817, 17851, |
| | 9679 | 16967, 17001, | 17273, 17307, | 17579, 17613, | 10879 | 17885, 17919, |
| | | 17035 | 17341 | 17647 | | 17953 |
| 7672 | 7923, | 9826, 9860, | 10098, 10132, | 10370, 10404, | 10642, | 10914, 10948, |
| | 8174, | 9894, 9928, | 10166, 10200, | 10438, 10472, | 10676, | 10982, 11016, |
| | 8425, | 9962, 9996, | 10234, 10268, | 10506, 10540, | 10710, | 11050, 11084, |
| | 8676, | 10030, 10064, | 10302, 10336, | 10574, 10608, | 10744, | 11118, 11152, |
| | 8927, | 16764, 16798, | 17070, 17104, | 17376, 17410, | 10778, | 17682, 17716, |
| | 9178, | 16832, 16866, | 17138, 17172, | 17444, 17478, | 10812, | 17750, 17784, |
| | 9429, | 16900, 16934, | 17206, 17240, | 17512, 17546, | 10846, | 17818, 17852, |
| | 9680 | 16968, 17002, | 17274, 17308, | 17580, 17614, | 10880 | 17886, 17920, |
| | | 17036 | 17342 | 17648 | | 17954 |
| 7690 | 7941, | 9827, 9861, | 10099, 10133, | 10371, 10405, | 10643, | 10915, 10949, |
| | 8192, | 9895, 9929, | 10167, 10201, | 10439, 10473, | 10677, | 10983, 11017, |
| | 8443, | 9963, 9997, | 10235, 10269, | 10507, 10541, | 10711, | 11051, 11085, |
| | 8694, | 10031, 10065, | 10303, 10337, | 10575, 10609, | 10745, | 11119, 11153, |
| | 8945, | 16765, 16799, | 17071, 17105, | 17377, 17411, | 10779, | 17683, 17717, |
| | 9196, | 16833, 16867, | 17139, 17173, | 17445, 17479, | 10813, | 17751, 17785, |
| | 9447, | 16901, 16935, | 17207, 17241, | 17513, 17547, | 10847, | 17819, 17853, |
| | 9698 | 16969, 17003, | 17275, 17309, | 17581, 17615, | 10881 | 17887, 17921, |
| | | 17037 | 17343 | 17649 | | 17955 |
| 7692 | 7943, | 9828, 9862, | 10100, 10134, | 10372, 10406, | 10644, | 10916, 10950, |
| | 8194, | 9896, 9930, | 10168, 10202, | 10440, 10474, | 10678, | 10984, 11018, |
| | 8445, | 9964, 9998, | 10236, 10270, | 10508, 10542, | 10712, | 11052, 11086, |
| | 8696, | 10032, 10066, | 10304, 10338, | 10576, 10610, | 10746, | 11120, 11154, |
| | 8947, | 16766, 16800, | 17072, 17106, | 17378, 17412, | 10780, | 17684, 17718, |
| | 9198, | 16834, 16868, | 17140, 17174, | 17446, 17480, | 10814, | 17752, 17786, |
| | 9449, | 16902, 16936, | 17208, 17242, | 17514, 17548, | 10848, | 17820, 17854, |
| | 9700 | 16970, 17004, | 17276, 17310, | 17582, 17616, | 10882 | 17888, 17922, |
| | | 17038 | 17344 | 17650 | | 17956 |
| 7695 | 7946, | 9829, 9863, | 10101, 10135, | 10373, 10407, | 10645, | 10917, 10951, |
| | 8197, | 9897, 9931, | 10169, 10203, | 10441, 10475, | 10679, | 10985, 11019, |
| | 8448, | 9965, 9999, | 10237, 10271, | 10509, 10543, | 10713, | 11053, 11087, |
| | 8699, | 10033, 10067, | 10305, 10339, | 10577, 10611, | 10747, | 11121, 11155, |
| | 8950, | 16767, 16801, | 17073, 17107, | 17379, 17413, | 10781, | 17685, 17719, |
| | 9201, | 16835, 16869, | 17141, 17175, | 17447, 17481, | 10815, | 17753, 17787, |
| | 9452, | 16903, 16937, | 17209, 17243, | 17515, 17549, | 10849, | 17821, 17855, |
| | 9703 | 16971, 17005, | 17277, 17311, | 17583, 17617, | 10883 | 17889, 17923, |
| | | 17039 | 17345 | 17651 | | 17957 |
| 7701 | 7952, | 9830, 9864, | 10102, 10136, | 10374, 10408, | 10646, | 10918, 10952, |
| | 8203, | 9898, 9932, | 10170, 10204, | 10442, 10476, | 10680, | 10986, 11020, |
| | 8454, | 9966, 10000, | 10238, 10272, | 10510, 10544, | 10714, | 11054, 11088, |
| | 8705, | 10034, 10068, | 10306, 10340, | 10578, 10612, | 10748, | 11122, 11156, |
| | 8956, | 16768, 16802, | 17074, 17108, | 17380, 17414, | 10782, | 17686, 17720, |
| | 9207, | 16836, 16870, | 17142, 17176, | 17448, 17482, | 10816, | 17754, 17788, |
| | 9458, | 16904, 16938, | 17210, 17244, | 17516, 17550, | 10850, | 17822, 17856, |
| | 9709 | 16972, 17006, | 17278, 17312, | 17584, 17618, | 10884 | 17890, 17924, |
| | | 17040 | 17346 | 17652 | | 17958 |
| 7704 | 7955, | 9831, 9865, | 10103, 10137, | 10375, 10409, | 10647, | 10919, 10953, |
| | 8206, | 9899, 9933, | 10171, 10205, | 10443, 10477, | 10681, | 10987, 11021, |
| | 8457, | 9967, 10001, | 10239, 10273, | 10511, 10545, | 10715, | 11055, 11089, |
| | 8708, | 10035, 10069, | 10307, 10341, | 10579, 10613, | 10749, | 11123, 11157, |
| | 8959, | 16769, 16803, | 17075, 17109, | 17381, 17415, | 10783, | 17687, 17721, |
| | 9210, | 16837, 16871, | 17143, 17177, | 17449, 17483, | 10817, | 17755, 17789, |
| | 9461, | 16905, 16939, | 17211, 17245, | 17517, 17551, | 10851, | 17823, 17857, |
| | 9712 | 16973, 17007, | 17279, 17313, | 17585, 17619, | 10885 | 17891, 17925, |
| | | 17041 | 17347 | 17653 | | 17959 |
| 7747 | 7998, | 9832, 9866, | 10104, 10138, | 10376, 10410, | 10648, | 10920, 10954, |
| | 8249, | 9900, 9934, | 10172, 10206, | 10444, 10478, | 10682, | 10988, 11022, |
| | 8500, | 9968, 10002, | 10240, 10274, | 10512, 10546, | 10716, | 11056, 11090, |
| | 8751, | 10036, 10070, | 10308, 10342, | 10580, 10614, | 10750, | 11124, 11158, |
| | 9002, | 16770, 16804, | 17076, 17110, | 17382, 17416, | 10784, | 17688, 17722, |
| | 9253, | 16838, 16872, | 17144, 17178, | 17450, 17484, | 10818, | 17756, 17790, |
| | 9504, | 16906, 16940, | 17212, 17246, | 17518, 17552, | 10852, | 17824, 17858, |
| | 9755 | 16974, 17008, | 17280, 17314, | 17586, 17620, | 10886 | 17892, 17926, |
| | | 17042 | 17348 | 17654 | | |
| | | | | | | 17960 |
| 7757 | 8008, | 9833, 9867, | 10105, 10139, | 10377, 10411, | 10649, | 10921, 10955, |
| | 8259, | 9901, 9935, | 10173, 10207, | 10445, 10479, | 10683, | 10989, 11023, |
| | 8510, | 9969, 10003, | 10241, 10275, | 10513, 10547, | 10717, | 11057, 11091, |
| | 8761, | 10037, 10071, | 10309, 10343, | 10581, 10615, | 10751, | 11125, 11159, |
| | 9012, | 16771, 16805, | 17077, 17111, | 17383, 17417, | 10785, | 17689, 17723, |
| | 9263, | 16839, 16873, | 17145, 17179, | 17451, 17485, | 10819, | 17757, 17791, |
| | 9514, | 16907, 16941, | 17213, 17247, | 17519, 17553, | 10853, | 17825, 17859, |
| | 9765 | 16975, 17009, | 17281, 17315, | 17587, 17621, | 10887 | 17893, 17927, |
| | | 17043 | 17349 | 17655 | | 17961 |
| 7763 | 8014, | 9834, 9868, | 10106, 10140, | 10378, 10412, | 10650, | 10922, 10956, |
| | 8265, | 9902, 9936, | 10174, 10208, | 10446, 10480, | 10684, | 10990, 11024, |
| | 8516, | 9970, 10004, | 10242, 10276, | 10514, 10548, | 10718, | 11058, 11092, |
| | 8767, | 10038, 10072, | 10310, 10344, | 10582, 10616, | 10752, | 11126, 11160, |
| | 9018, | 16772, 16806, | 17078, 17112, | 17384, 17418, | 10786, | 17690, 17724, |
| | 9269, | 16840, 16874, | 17146, 17180, | 17452, 17486, | 10820, | 17758, 17792, |
| | 9520, | 16908, 16942, | 17214, 17248, | 17520, 17554, | 10854, | 17826, 17860, |
| | 9771 | 16976, 17010, | 17282, 17316, | 17588, 17622, | 10888 | 17894, 17928, |
| | | 17044 | 17350 | 17656 | | 17962 |
| 7768 | 8019, | 9835, 9869, | 10107, 10141, | 10379, 10413, | 10651, | 10923, 10957, |
| | 8270, | 9903, 9937, | 10175, 10209, | 10447, 10481, | 10685, | 10991, 11025, |
| | 8521, | 9971, 10005, | 10243, 10277, | 10515, 10549, | 10719, | 11059, 11093, |
| | 8772, | 10039, 10073, | 10311, 10345, | 10583, 10617, | 10753, | 11127, 11161, |
| | 9023, | 16773, 16807, | 17079, 17113, | 17385, 17419, | 10787, | 17691, 17725, |
| | 9274, | 16841, 16875, | 17147, 17181, | 17453, 17487, | 10821, | 17759, 17793, |
| | 9525, | 16909, 16943, | 17215, 17249, | 17521, 17555, | 10855, | 17827, 17861, |
| | 9776 | 16977, 17011, | 17283, 17317, | 17589, 17623, | 10889 | 17895, 17929, |
| | | 17045 | 17351 | 17657 | | 17963 |
| 7779 | 8030, | 9836, 9870, | 10108, 10142, | 10380, 10414, | 10652, | 10924, 10958, |
| | 8281, | 9904, 9938, | 10176, 10210, | 10448, 10482, | 10686, | 10992, 11026, |
| | 8532, | 9972, 10006, | 10244, 10278, | 10516, 10550, | 10720, | 11060, 11094, |
| | 8783, | 10040, 10074, | 10312, 10346, | 10584, 10618, | 10754, | 11128, 11162, |
| | 9034, | 16774, 16808, | 17080, 17114, | 17386, 17420, | 10788, | 17692, 17726, |
| | 9285, | 16842, 16876, | 17148, 17182, | 17454, 17488, | 10822, | 17760, 17794, |
| | 9536, | 16910, 16944, | 17216, 17250, | 17522, 17556, | 10856, | 17828, 17862, |
| | 9787 | 16978, 17012, | 17284, 17318, | 17590, 17624, | 10890 | 17896, 17930, |
| | | 17046 | 17352 | 17658 | | 17964 |
| 7781 | 8032, | 9837, 9871, | 10109, 10143, | 10381, 10415, | 10653, | 10925, 10959, |
| | 8283, | 9905, 9939, | 10177, 10211, | 10449, 10483, | 10687, | 10993, 11027, |
| | 8534, | 9973, 10007, | 10245, 10279, | 10517, 10551, | 10721, | 11061, 11095, |
| | 8785, | 10041, 10075, | 10313, 10347, | 10585, 10619, | 10755, | 11129, 11163, |
| | 9036, | 16775, 16809, | 17081, 17115, | 17387, 17421, | 10789, | 17693, 17727, |
| | 9287, | 16843, 16877, | 17149, 17183, | 17455, 17489, | 10823, | 17761, 17795, |
| | 9538, | 16911, 16945, | 17217, 17251, | 17523, 17557, | 10857, | 17829, 17863, |
| | 9789 | 16979, 17013, | 17285, 17319, | 17591, 17625, | 10891 | 17897, 17931, |
| | | 17047 | 17353 | 17659 | | 17965 |
| 7782 | 8033, | 9838, 9872, | 10110, 10144, | 10382, 10416, | 10654, | 10926, 10960, |
| | 8284, | 9906, 9940, | 10178, 10212, | 10450, 10484, | 10688, | 10994, 11028, |
| | 8535, | 9974, 10008, | 10246, 10280, | 10518, 10552, | 10722, | 11062, 11096, |
| | 8786, | 10042, 10076, | 10314, 10348, | 10586, 10620, | 10756, | 11130, 11164, |
| | 9037, | 16776, 16810, | 17082, 17116, | 17388, 17422, | 10790, | 17694, 17728, |
| | 9288, | 16844, 16878, | 17150, 17184, | 17456, 17490, | 10824, | 17762, 17796, |
| | 9539, | 16912, 16946, | 17218, 17252, | 17524, 17558, | 10858, | 17830, 17864, |
| | 9790 | 16980, 17014, | 17286, 17320, | 17592, 17626, | 10892 | 17898, 17932, |
| | | 17048 | 17354 | 17660 | | 17966 |
| 7788 | 8039, | 9839, 9873, | 10111, 10145, | 10383, 10417, | 10655, | 10927, 10961, |
| | 8290, | 9907, 9941, | 10179, 10213, | 10451, 10485, | 10689, | 10995, 11029, |
| | 8541, | 9975, 10009, | 10247, 10281, | 10519, 10553, | 10723, | 11063, 11097, |
| | 8792, | 10043, 10077, | 10315, 10349, | 10587, 10621, | 10757, | 11131, 11165, |
| | 9043, | 16777, 16811, | 17083, 17117, | 17389, 17423, | 10791, | 17695, 17729, |
| | 9294, | 16845, 16879, | 17151, 17185, | 17457, 17491, | 10825, | 17763, 17797, |
| | 9545, | 16913, 16947, | 17219, 17253, | 17525, 17559, | 10859, | 17831, 17865, |
| | 9796 | 16981, 17015, | 17287, 17321, | 17593, 17627, | 10893 | 17899, 17933, |
| | | 17049 | 17355 | 17661 | | 17967 |

TABLE 4.1
Preferred LASV Nucleoprotein (SP-HsPLAT NP) constructs

| | | RNA designs and SEQ ID NOs | | | | |
|---|---|---|---|---|---|---|
| PRT | CDS | a-1 | a-2 | b-4 | i-2 | i-3 |
| 17968 | 18002, | 18104, 18138, | 18410, 18444, | 18716, 18750, | | 19022, 19056, |
| | 18036, | 18172, 18206, | 18478, 18512, | 18784, 18818, | | 19090, 19124, |
| | 18070 | 18240, 18274, | 18546, 18580, | 18852, 18886, | | 19158, 19192, |
| | | 18308, 18342, | 18614, 18648, | 18920, 18954, | | 19226, 19260, |
| | | 18376 | 18682 | 18988 | | 19294 |
| 17969 | 18003, | 18105, 18139, | 18411, 18445, | 18717, 18751, | | 19023, 19057, |
| | 18037, | 18173, 18207, | 18479, 18513, | 18785, 18819, | | 19091, 19125, |
| | 18071 | 18241, 18275, | 18547, 18581, | 18853, 18887, | | 19159, 19193, |
| | | 18309, 18343, | 18615, 18649, | 18921, 18955, | | 19227, 19261, |
| | | 18377 | 18683 | 18989 | | 19295 |
| 17970 | 18004, | 18106, 18140, | 18412, 18446, | 18718, 18752, | | 19024, 19058, |
| | 18038, | 18174, 18208, | 18480, 18514, | 18786, 18820, | | 19092, 19126, |
| | 18072 | 18242, 18276, | 18548, 18582, | 18854, 18888, | | 19160, 19194, |
| | | 18310, 18344, | 18616, 18650, | 18922, 18956, | | 19228, 19262, |
| | | 18378 | 18684 | 18990 | | 19296 |
| 17971 | 18005, | 18107, 18141, | 18413, 18447, | 18719, 18753, | | 19025, 19059, |
| | 18039, | 18175, 18209, | 18481, 18515, | 18787, 18821, | | 19093, 19127, |
| | 18073 | 18243, 18277, | 18549, 18583, | 18855, 18889, | | 19161, 19195, |
| | | 18311, 18345, | 18617, 18651, | 18923, 18957, | | 19229, 19263, |
| | | 18379 | 18685 | 18991 | | 19297 |
| 17972 | 18006, | 18108, 18142, | 18414, 18448, | 18720, 18754, | | 19026, 19060, |
| | 18040, | 18176, 18210, | 18482, 18516, | 18788, 18822, | | 19094, 19128, |
| | 18074 | 18244, 18278, | 18550, 18584, | 18856, 18890, | | 19162, 19196, |
| | | 18312, 18346, | 18618, 18652, | 18924, 18958, | | 19230, 19264, |
| | | 18380 | 18686 | 18992 | | 19298 |
| 17973 | 18007, | 18109, 18143, | 18415, 18449, | 18721, 18755, | | 19027, 19061, |
| | 18041, | 18177, 18211, | 18483, 18517, | 18789, 18823, | | 19095, 19129, |
| | 18075 | 18245, 18279, | 18551, 18585, | 18857, 18891, | | 19163, 19197, |
| | | 18313, 18347, | 18619, 18653, | 18925, 18959, | | 19231, 19265, |
| | | 18381 | 18687 | 18993 | | 19299 |
| 17974 | 18008, | 18110, 18144, | 18416, 18450, | 18722, 18756, | | 19028, 19062, |
| | 18042, | 18178, 18212, | 18484, 18518, | 18790, 18824, | | 19096, 19130, |
| | 18076 | 18246, 18280, | 18552, 18586, | 18858, 18892, | | 19164, 19198, |
| | | 18314, 18348, | 18620, 18654, | 18926, 18960, | | 19232, 19266, |
| | | 18382 | 18688 | 18994 | | 19300 |
| 17975 | 18009, | 18111, 18145, | 18417, 18451, | 18723, 18757, | | 19029, 19063, |
| | 18043, | 18179, 18213, | 18485, 18519, | 18791, 18825, | | 19097, 19131, |
| | 18077 | 18247, 18281, | 18553, 18587, | 18859, 18893, | | 19165, 19199, |
| | | 18315, 18349, | 18621, 18655, | 18927, 18961, | | 19233, 19267, |
| | | 18383 | 18689 | 18995 | | 19301 |
| 17976 | 18010, | 18112, 18146, | 18418, 18452, | 18724, 18758, | | 19030, 19064, |
| | 18044, | 18180, 18214, | 18486, 18520, | 18792, 18826, | | 19098, 19132, |
| | 18078 | 18248, 18282, | 18554, 18588, | 18860, 18894, | | 19166, 19200, |
| | | 18316, 18350, | 18622, 18656, | 18928, 18962, | | 19234, 19268, |
| | | 18384 | 18690 | 18996 | | 19302 |
| 17977 | 18011, | 18113, 18147, | 18419, 18453, | 18725, 18759, | | 19031, 19065, |
| | 18045, | 18181, 18215, | 18487, 18521, | 18793, 18827, | | 19099, 19133, |
| | 18079 | 18249, 18283, | 18555, 18589, | 18861, 18895, | | 19167, 19201, |
| | | 18317, 18351, | 18623, 18657, | 18929, 18963, | | 19235, 19269, |
| | | 18385 | 18691 | 18997 | | 19303 |
| 17978 | 18012, | 18114, 18148, | 18420, 18454, | 18726, 18760, | | 19032, 19066, |
| | 18046, | 18182, 18216, | 18488, 18522, | 18794, 18828, | | 19100, 19134, |
| | 18080 | 18250, 18284, | 18556, 18590, | 18862, 18896, | | 19168, 19202, |
| | | 18318, 18352, | 18624, 18658, | 18930, 18964, | | 19236, 19270, |
| | | 18386 | 18692 | 18998 | | 19304 |
| 17979 | 18013, | 18115, 18149, | 18421, 18455, | 18727, 18761, | | 19033, 19067, |
| | 18047, | 18183, 18217, | 18489, 18523, | 18795, 18829, | | 19101, 19135, |
| | 18081 | 18251, 18285, | 18557, 18591, | 18863, 18897, | | 19169, 19203, |
| | | 18319, 18353, | 18625, 18659, | 18931, 18965, | | 19237, 19271, |
| | | 18387 | 18693 | 18999 | | 19305 |
| 17980 | 18014, | 18116, 18150, | 18422, 18456, | 18728, 18762, | | 19034, 19068, |
| | 18048, | 18184, 18218, | 18490, 18524, | 18796, 18830, | | 19102, 19136, |
| | 18082 | 18252, 18286, | 18558, 18592, | 18864, 18898, | | 19170, 19204, |
| | | 18320, 18354, | 18626, 18660, | 18932, 18966, | | 19238, 19272, |
| | | 18388 | 18694 | 19000 | | 19306 |
| 17981 | 18015, | 18117, 18151, | 18423, 18457, | 18729, 18763, | | 19035, 19069, |
| | 18049, | 18185, 18219, | 18491, 18525, | 18797, 18831, | | 19103, 19137, |
| | 18083 | 18253, 18287, | 18559, 18593, | 18865, 18899, | | 19171, 19205, |
| | | 18321, 18355, | 18627, 18661, | 18933, 18967, | | 19239, 19273, |
| | | 18389 | 18695 | 19001 | | 19307 |
| 17982 | 18016, | 18118, 18152, | 18424, 18458, | 18730, 18764, | | 19036, 19070, |
| | 18050, | 18186, 18220, | 18492, 18526, | 18798, 18832, | | 19104, 19138, |
| | 18084 | 18254, 18288, | 18560, 18594, | 18866, 18900, | | 19172, 19206, |
| | | 18322, 18356, | 18628, 18662, | 18934, 18968, | | 19240, 19274, |
| | | 18390 | 18696 | 19002 | | 19308 |
| 17983 | 18017, | 18119, 18153, | 18425, 18459, | 18731, 18765, | | 19037, 19071, |
| | 18051, | 18187, 18221, | 18493, 18527, | 18799, 18833, | | 19105, 19139, |
| | 18085 | 18255, 18289, | 18561, 18595, | 18867, 18901, | | 19173, 19207, |
| | | 18323, 18357, | 18629, 18663, | 18935, 18969, | | 19241, 19275, |
| | | 18391 | 18697 | 19003 | | 19309 |
| 17984 | 18018, | 18120, 18154, | 18426, 18460, | 18732, 18766, | | 19038, 19072, |
| | 18052, | 18188, 18222, | 18494, 18528, | 18800, 18834, | | 19106, 19140, |
| | 18086 | 18256, 18290, | 18562, 18596, | 18868, 18902, | | 19174, 19208, |
| | | 18324, 18358, | 18630, 18664, | 18936, 18970, | | 19242, 19276, |
| | | 18392 | 18698 | 19004 | | 19310 |
| 17985 | 18019, | 18121, 18155, | 18427, 18461, | 18733, 18767, | | 19039, 19073, |
| | 18053, | 18189, 18223, | 18495, 18529, | 18801, 18835, | | 19107, 19141, |
| | 18087 | 18257, 18291, | 18563, 18597, | 18869, 18903, | | 19175, 19209, |
| | | 18325, 18359, | 18631, 18665, | 18937, 18971, | | 19243, 19277, |
| | | 18393 | 18699 | 19005 | | 19311 |
| 17986 | 18020, | 18122, 18156, | 18428, 18462, | 18734, 18768, | | 19040, 19074, |
| | 18054, | 18190, 18224, | 18496, 18530, | 18802, 18836, | | 19108, 19142, |
| | 18088 | 18258, 18292, | 18564, 18598, | 18870, 18904, | | 19176, 19210, |
| | | 18326, 18360, | 18632, 18666, | 18938, 18972, | | 19244, 19278, |
| | | 18394 | 18700 | 19006 | | 19312 |
| 17987 | 18021, | 18123, 18157, | 18429, 18463, | 18735, 18769, | | 19041, 19075, |
| | 18055, | 18191, 18225, | 18497, 18531, | 18803, 18837, | | 19109, 19143, |
| | 18089 | 18259, 18293, | 18565, 18599, | 18871, 18905, | | 19177, 19211, |
| | | 18327, 18361, | 18633, 18667, | 18939, 18973, | | 19245, 19279, |
| | | 18395 | 18701 | 19007 | | 19313 |
| 17988 | 18022, | 18124, 18158, | 18430, 18464, | 18736, 18770, | | 19042, 19076, |
| | 18056, | 18192, 18226, | 18498, 18532, | 18804, 18838, | | 19110, 19144, |
| | 18090 | 18260, 18294, | 18566, 18600, | 18872, 18906, | | 19178, 19212, |
| | | 18328, 18362, | 18634, 18668, | 18940, 18974, | | 19246, 19280, |
| | | 18396 | 18702 | 19008 | | 19314 |
| 17989 | 18023, | 18125, 18159, | 18431, 18465, | 18737, 18771, | | 19043, 19077, |
| | 18057, | 18193, 18227, | 18499, 18533, | 18805, 18839, | | 19111, 19145, |
| | 18091 | 18261, 18295, | 18567, 18601, | 18873, 18907, | | 19179, 19213, |
| | | 18329, 18363, | 18635, 18669, | 18941, 18975, | | 19247, 19281, |
| | | 18397 | 18703 | 19009 | | 19315 |
| 17990 | 18024, | 18126, 18160, | 18432, 18466, | 18738, 18772, | | 19044, 19078, |
| | 18058, | 18194, 18228, | 18500, 18534, | 18806, 18840, | | 19112, 19146, |
| | 18092 | 18262, 18296, | 18568, 18602, | 18874, 18908, | | 19180, 19214, |
| | | 18330, 18364, | 18636, 18670, | 18942, 18976, | | 19248, 19282, |
| | | 18398 | 18704 | 19010 | | 19316 |
| 17991 | 18025, | 18127, 18161, | 18433, 18467, | 18739, 18773, | | 19045, 19079, |
| | 18059, | 18195, 18229, | 18501, 18535, | 18807, 18841, | | 19113, 19147, |
| | 18093 | 18263, 18297, | 18569, 18603, | 18875, 18909, | | 19181, 19215, |
| | | 18331, 18365, | 18637, 18671, | 18943, 18977, | | 19249, 19283, |
| | | 18399 | 18705 | 19011 | | 19317 |
| 17992 | 18026, | 18128, 18162, | 18434, 18468, | 18740, 18774, | | 19046, 19080, |
| | 18060, | 18196, 18230, | 18502, 18536, | 18808, 18842, | | 19114, 19148, |
| | 18094 | 18264, 18298, | 18570, 18604, | 18876, 18910, | | 19182, 19216, |
| | | 18332, 18366, | 18638, 18672, | 18944, 18978, | | 19250, 19284, |
| | | 18400 | 18706 | 19012 | | 19318 |
| 17993 | 18027, | 18129, 18163, | 18435, 18469, | 18741, 18775, | | 19047, 19081, |
| | 18061, | 18197, 18231, | 18503, 18537, | 18809, 18843, | | 19115, 19149, |
| | 18095 | 18265, 18299, | 18571, 18605, | 18877, 18911, | | 19183, 19217, |
| | | 18333, 18367, | 18639, 18673, | 18945, 18979, | | 19251, 19285, |
| | | 18401 | 18707 | 19013 | | 19319 |
| 17994 | 18028, | 18130, 18164, | 18436, 18470, | 18742, 18776, | | 19048, 19082, |
| | 18062, | 18198, 18232, | 18504, 18538, | 18810, 18844, | | 19116, 19150, |
| | 18096 | 18266, 18300, | 18572, 18606, | 18878, 18912, | | 19184, 19218, |
| | | 18334, 18368, | 18640, 18674, | 18946, 18980, | | 19252, 19286, |
| | | 18402 | 18708 | 19014 | | 19320 |
| 17995 | 18029, | 18131, 18165, | 18437, 18471, | 18743, 18777, | | 19049, 19083, |
| | 18063, | 18199, 18233, | 18505, 18539, | 18811, 18845, | | 19117, 19151, |
| | 18097 | 18267, 18301, | 18573, 18607, | 18879, 18913, | | 19185, 19219, |
| | | 18335, 18369, | 18641, 18675, | 18947, 18981, | | 19253, 19287, |
| | | 18403 | 18709 | 19015 | | 19321 |
| 17996 | 18030, | 18132, 18166, | 18438, 18472, | 18744, 18778, | | 19050, 19084, |
| | 18064, | 18200, 18234, | 18506, 18540, | 18812, 18846, | | 19118, 19152, |
| | 18098 | 18268, 18302, | 18574, 18608, | 18880, 18914, | | 19186, 19220, |
| | | 18336, 18370, | 18642, 18676, | 18948, 18982, | | 19254, 19288, |
| | | 18404 | 18710 | 19016 | | 19322 |
| 17997 | 18031, | 18133, 18167, | 18439, 18473, | 18745, 18779, | | 19051, 19085, |
| | 18065, | 18201, 18235, | 18507, 18541, | 18813, 18847, | | 19119, 19153, |
| | 18099 | 18269, 18303, | 18575, 18609, | 18881, 18915, | | 19187, 19221, |
| | | 18337, 18371, | 18643, 18677, | 18949, 18983, | | 19255, 19289, |
| | | 18405 | 18711 | 19017 | | 19323 |
| 17998 | 18032, | 18134, 18168, | 18440, 18474, | 18746, 18780, | | 19052, 19086, |
| | 18066, | 18202, 18236, | 18508, 18542, | 18814, 18848, | | 19120, 19154, |
| | 18100 | 18270, 18304, | 18576, 18610, | 18882, 18916, | | 19188, 19222, |
| | | 18338, 18372, | 18644, 18678, | 18950, 18984, | | 19256, 19290, |
| | | 18406 | 18712 | 19018 | | 19324 |
| 17999 | 18033, | 18135, 18169, | 18441, 18475, | 18747, 18781, | | 19053, 19087, |
| | 18067, | 18203, 18237, | 18509, 18543, | 18815, 18849, | | 19121, 19155, |
| | 18101 | 18271, 18305, | 18577, 18611, | 18883, 18917, | | 19189, 19223, |
| | | 18339, 18373, | 18645, 18679, | 18951, 18985, | | 19257, 19291, |
| | | 18407 | 18713 | 19019 | | 19325 |
| 18000 | 18034, | 18136, 18170, | 18442, 18476, | 18748, 18782, | | 19054, 19088, |
| | 18068, | 18204, 18238, | 18510, 18544, | 18816, 18850, | | 19122, 19156, |
| | 18102 | 18272, 18306, | 18578, 18612, | 18884, 18918, | | 19190, 19224, |
| | | 18340, 18374, | 18646, 18680, | 18952, 18986, | | 19258, 19292, |
| | | 18408 | 18714 | 19020 | | 19326 |
| 18001 | 18035, | 18137, 18171, | 18443, 18477, | 18749, 18783, | | 19055, 19089, |
| | 18069, | 18205, 18239, | 18511, 18545, | 18817, 18851, | | 19123, 19157, |
| | 18103 | 18273, 18307, | 18579, 18613, | 18885, 18919, | | 19191, 19225, |
| | | 18341, 18375, | 18647, 18681, | 18953, 18987, | | 19259, 19293, |
| | | 18409 | 18715 | 19021 | | 19327 |

TABLE 4.2
Preferred LASV Nucleoprotein (SP-HsALB NP) constructs

| | | RNA designs and SEQ ID NOs | | | | |
|---|---|---|---|---|---|---|
| PRT | CDS | a-1 | a-2 | b-4 | i-2 | i-3 |
| 19328 | 19362, | 19464, 19498, | 19770, 19804, | 20076, 20110, | | 20382, 20416, |
| | 19396, | 19532, 19566, | 19838, 19872, | 20144, 20178, | | 20450, 20484, |
| | 19430 | 19600, 19634, | 19906, 19940, | 20212, 20246, | | 20518, 20552, |
| | | 19668, 19702, | 19974, 20008, | 20280, 20314, | | 20586, 20620, |
| | | 19736 | 20042 | 20348 | | 20654 |
| 19329 | 19363, | 19465, 19499, | 19771, 19805, | 20077, 20111, | | 20383, 20417, |
| | 19397, | 19533, 19567, | 19839, 19873, | 20145, 20179, | | 20451, 20485, |
| | 19431 | 19601, 19635, | 19907, 19941, | 20213, 20247, | | 20519, 20553, |
| | | 19669, 19703, | 19975, 20009, | 20281, 20315, | | 20587, 20621, |
| | | 19737 | 20043 | 20349 | | 20655 |
| 19330 | 19364, | 19466, 19500, | 19772, 19806, | 20078, 20112, | | 20384, 20418, |
| | 19398, | 19534, 19568, | 19840, 19874, | 20146, 20180, | | 20452, 20486, |
| | 19432 | 19602, 19636, | 19908, 19942, | 20214, 20248, | | 20520, 20554, |
| | | 19670, 19704, | 19976, 20010, | 20282, 20316, | | 20588, 20622, |
| | | 19738 | 20044 | 20350 | | 20656 |
| 19331 | 19365, | 19467, 19501, | 19773, 19807, | 20079, 20113, | | 20385, 20419, |
| | 19399, | 19535, 19569, | 19841, 19875, | 20147, 20181, | | 20453, 20487, |
| | 19433 | 19603, 19637, | 19909, 19943, | 20215, 20249, | | 20521, 20555, |
| | | 19671, 19705, | 19977, 20011, | 20283, 20317, | | 20589, 20623, |
| | | 19739 | 20045 | 20351 | | 20657 |
| 19332 | 19366, | 19468, 19502, | 19774, 19808, | 20080, 20114, | | 20386, 20420, |
| | 19400, | 19536, 19570, | 19842, 19876, | 20148, 20182, | | 20454, 20488, |
| | 19434 | 19604, 19638, | 19910, 19944, | 20216, 20250, | | 20522, 20556, |
| | | 19672, 19706, | 19978, 20012, | 20284, 20318, | | 20590, 20624, |
| | | 19740 | 20046 | 20352 | | 20658 |
| 19333 | 19367, | 19469, 19503, | 19775, 19809, | 20081, 20115, | | 20387, 20421, |
| | 19401, | 19537, 19571, | 19843, 19877, | 20149, 20183, | | 20455, 20489, |
| | 19435 | 19605, 19639, | 19911, 19945, | 20217, 20251, | | 20523, 20557, |
| | | 19673, 19707, | 19979, 20013, | 20285, 20319, | | 20591, 20625, |
| | | 19741 | 20047 | 20353 | | 20659 |
| 19334 | 19368, | 19470, 19504, | 19776, 19810, | 20082, 20116, | | 20388, 20422, |
| | 19402, | 19538, 19572, | 19844, 19878, | 20150, 20184, | | 20456, 20490, |
| | 19436 | 19606, 19640, | 19912, 19946, | 20218, 20252, | | 20524, 20558, |
| | | 19674, 19708, | 19980, 20014, | 20286, 20320, | | 20592, 20626, |
| | | 19742 | 20048 | 20354 | | 20660 |
| 19335 | 19369, | 19471, 19505, | 19777, 19811, | 20083, 20117, | | 20389, 20423, |
| | 19403, | 19539, 19573, | 19845, 19879, | 20151, 20185, | | 20457, 20491, |
| | 19437 | 19607, 19641, | 19913, 19947, | 20219, 20253, | | 20525, 20559, |
| | | 19675, 19709, | 19981, 20015, | 20287, 20321, | | 20593, 20627, |
| | | 19743 | 20049 | 20355 | | 20661 |
| 19336 | 19370, | 19472, 19506, | 19778, 19812, | 20084, 20118, | | 20390, 20424, |
| | 19404, | 19540, 19574, | 19846, 19880, | 20152, 20186, | | 20458, 20492, |
| | 19438 | 19608, 19642, | 19914, 19948, | 20220, 20254, | | 20526, 20560, |
| | | 19676, 19710, | 19982, 20016, | 20288, 20322, | | 20594, 20628, |
| | | 19744 | 20050 | 20356 | | 20662 |
| 19337 | 19371, | 19473, 19507, | 19779, 19813, | 20085, 20119, | | 20391, 20425, |
| | 19405, | 19541, 19575, | 19847, 19881, | 20153, 20187, | | 20459, 20493, |
| | 19439 | 19609, 19643, | 19915, 19949, | 20221, 20255, | | 20527, 20561, |
| | | 19677, 19711, | 19983, 20017, | 20289, 20323, | | 20595, 20629, |
| | | 19745 | 20051 | 20357 | | 20663 |
| 19338 | 19372, | 19474, 19508, | 19780, 19814, | 20086, 20120, | | 20392, 20426, |
| | 19406, | 19542, 19576, | 19848, 19882, | 20154, 20188, | | 20460, 20494, |
| | 19440 | 19610, 19644, | 19916, 19950, | 20222, 20256, | | 20528, 20562, |
| | | 19678, 19712, | 19984, 20018, | 20290, 20324, | | 20596, 20630, |
| | | 19746 | 20052 | 20358 | | 20664 |
| 19339 | 19373, | 19475, 19509, | 19781, 19815, | 20087, 20121, | | 20393, 20427, |
| | 19407, | 19543, 19577, | 19849, 19883, | 20155, 20189, | | 20461, 20495, |
| | 19441 | 19611, 19645, | 19917, 19951, | 20223, 20257, | | 20529, 20563, |
| | | 19679, 19713, | 19985, 20019, | 20291, 20325, | | 20597, 20631, |
| | | 19747 | 20053 | 20359 | | 20665 |
| 19340 | 19374, | 19476, 19510, | 19782, 19816, | 20088, 20122, | | 20394, 20428, |
| | 19408, | 19544, 19578, | 19850, 19884, | 20156, 20190, | | 20462, 20496, |
| | 19442 | 19612, 19646, | 19918, 19952, | 20224, 20258, | | 20530, 20564, |
| | | 19680, 19714, | 19986, 20020, | 20292, 20326, | | 20598, 20632, |
| | | 19748 | 20054 | 20360 | | 20666 |
| 19341 | 19375, | 19477, 19511, | 19783, 19817, | 20089, 20123, | | 20395, 20429, |
| | 19409, | 19545, 19579, | 19851, 19885, | 20157, 20191, | | 20463, 20497, |
| | 19443 | 19613, 19647, | 19919, 19953, | 20225, 20259, | | 20531, 20565, |
| | | 19681, 19715, | 19987, 20021, | 20293, 20327, | | 20599, 20633, |
| | | 19749 | 20055 | 20361 | | 20667 |
| 19342 | 19376, | 19478, 19512, | 19784, 19818, | 20090, 20124, | | 20396, 20430, |
| | 19410, | 19546, 19580, | 19852, 19886, | 20158, 20192, | | 20464, 20498, |
| | 19444 | 19614, 19648, | 19920, 19954, | 20226, 20260, | | 20532, 20566, |
| | | 19682, 19716, | 19988, 20022, | 20294, 20328, | | 20600, 20634, |
| | | 19750 | 20056 | 20362 | | 20668 |
| 19343 | 19377, | 19479, 19513, | 19785, 19819, | 20091, 20125, | | 20397, 20431, |
| | 19411, | 19547, 19581, | 19853, 19887, | 20159, 20193, | | 20465, 20499, |
| | 19445 | 19615, 19649, | 19921, 19955, | 20227, 20261, | | 20533, 20567, |
| | | 19683, 19717, | 19989, 20023, | 20295, 20329, | | 20601, 20635, |
| | | 19751 | 20057 | 20363 | | 20669 |
| 19344 | 19378, | 19480, 19514, | 19786, 19820, | 20092, 20126, | | 20398, 20432, |
| | 19412, | 19548, 19582, | 19854, 19888, | 20160, 20194, | | 20466, 20500, |
| | 19446 | 19616, 19650, | 19922, 19956, | 20228, 20262, | | 20534, 20568, |
| | | 19684, 19718, | 19990, 20024, | 20296, 20330, | | 20602, 20636, |
| | | 19752 | 20058 | 20364 | | 20670 |
| 19345 | 19379, | 19481, 19515, | 19787, 19821, | 20093, 20127, | | 20399, 20433, |
| | 19413, | 19549, 19583, | 19855, 19889, | 20161, 20195, | | 20467, 20501, |
| | 19447 | 19617, 19651, | 19923, 19957, | 20229, 20263, | | 20535, 20569, |
| | | 19685, 19719, | 19991, 20025, | 20297, 20331, | | 20603, 20637, |
| | | 19753 | 20059 | 20365 | | 20671 |
| 19346 | 19380, | 19482, 19516, | 19788, 19822, | 20094, 20128, | | 20400, 20434, |
| | 19414, | 19550, 19584, | 19856, 19890, | 20162, 20196, | | 20468, 20502, |
| | 19448 | 19618, 19652, | 19924, 19958, | 20230, 20264, | | 20536, 20570, |
| | | 19686, 19720, | 19992, 20026, | 20298, 20332, | | 20604, 20638, |
| | | 19754 | 20060 | 20366 | | 20672 |
| 19347 | 19381, | 19483, 19517, | 19789, 19823, | 20095, 20129, | | 20401, 20435, |
| | 19415, | 19551, 19585, | 19857, 19891, | 20163, 20197, | | 20469, 20503, |
| | 19449 | 19619, 19653, | 19925, 19959, | 20231, 20265, | | 20537, 20571, |
| | | 19687, 19721, | 19993, 20027, | 20299, 20333, | | 20605, 20639, |
| | | 19755 | 20061 | 20367 | | 20673 |
| 19348 | 19382, | 19484, 19518, | 19790, 19824, | 20096, 20130, | | 20402, 20436, |
| | 19416, | 19552, 19586, | 19858, 19892, | 20164, 20198, | | 20470, 20504, |
| | 19450 | 19620, 19654, | 19926, 19960, | 20232, 20266, | | 20538, 20572, |
| | | 19688, 19722, | 19994, 20028, | 20300, 20334, | | 20606, 20640, |
| | | 19756 | 20062 | 20368 | | 20674 |
| 19349 | 19383, | 19485, 19519, | 19791, 19825, | 20097, 20131, | | 20403, 20437, |
| | 19417, | 19553, 19587, | 19859, 19893, | 20165, 20199, | | 20471, 20505, |
| | 19451 | 19621, 19655, | 19927, 19961, | 20233, 20267, | | 20539, 20573, |
| | | 19689, 19723, | 19995, 20029, | 20301, 20335, | | 20607, 20641, |
| | | 19757 | 20063 | 20369 | | 20675 |
| 19350 | 19384, | 19486, 19520, | 19792, 19826, | 20098, 20132, | | 20404, 20438, |
| | 19418, | 19554, 19588, | 19860, 19894, | 20166, 20200, | | 20472, 20506, |
| | 19452 | 19622, 19656, | 19928, 19962, | 20234, 20268, | | 20540, 20574, |
| | | 19690, 19724, | 19996, 20030, | 20302, 20336, | | 20608, 20642, |
| | | 19758 | 20064 | 20370 | | 20676 |
| 19351 | 19385, | 19487, 19521, | 19793, 19827, | 20099, 20133, | | 20405, 20439, |
| | 19419, | 19555, 19589, | 19861, 19895, | 20167, 20201, | | 20473, 20507, |
| | 19453 | 19623, 19657, | 19929, 19963, | 20235, 20269, | | 20541, 20575, |
| | | 19691, 19725, | 19997, 20031, | 20303, 20337, | | 20609, 20643, |
| | | 19759 | 20065 | 20371 | | 20677 |
| 19352 | 19386, | 19488, 19522, | 19794, 19828, | 20100, 20134, | | 20406, 20440, |
| | 19420, | 19556, 19590, | 19862, 19896, | 20168, 20202, | | 20474, 20508, |
| | 19454 | 19624, 19658, | 19930, 19964, | 20236, 20270, | | 20542, 20576, |
| | | 19692, 19726, | 19998, 20032, | 20304, 20338, | | 20610, 20644, |
| | | 19760 | 20066 | 20372 | | 20678 |
| 19353 | 19387, | 19489, 19523, | 19795, 19829, | 20101, 20135, | | 20407, 20441, |
| | 19421, | 19557, 19591, | 19863, 19897, | 20169, 20203, | | 20475, 20509, |
| | 19455 | 19625, 19659, | 19931, 19965, | 20237, 20271, | | 20543, 20577, |
| | | 19693, 19727, | 19999, 20033, | 20305, 20339, | | 20611, 20645, |
| | | 19761 | 20067 | 20373 | | 20679 |
| 19354 | 19388, | 19490, 19524, | 19796, 19830, | 20102, 20136, | | 20408, 20442, |
| | 19422, | 19558, 19592, | 19864, 19898, | 20170, 20204, | | 20476, 20510, |
| | 19456 | 19626, 19660, | 19932, 19966, | 20238, 20272, | | 20544, 20578, |
| | | 19694, 19728, | 20000, 20034, | 20306, 20340, | | 20612, 20646, |
| | | 19762 | 20068 | 20374 | | 20680 |
| 19355 | 19389, | 19491, 19525, | 19797, 19831, | 20103, 20137, | | 20409, 20443, |
| | 19423, | 19559, 19593, | 19865, 19899, | 20171, 20205, | | 20477, 20511, |
| | 19457 | 19627, 19661, | 19933, 19967, | 20239, 20273, | | 20545, 20579, |
| | | 19695, 19729, | 20001, 20035, | 20307, 20341, | | 20613, 20647, |
| | | 19763 | 20069 | 20375 | | 20681 |
| 19356 | 19390, | 19492, 19526, | 19798, 19832, | 20104, 20138, | | 20410, 20444, |
| | 19424, | 19560, 19594, | 19866, 19900, | 20172, 20206, | | 20478, 20512, |
| | 19458 | 19628, 19662, | 19934, 19968, | 20240, 20274, | | 20546, 20580, |
| | | 19696, 19730, | 20002, 20036, | 20308, 20342, | | 20614, 20648, |
| | | 19764 | 20070 | 20376 | | 20682 |
| 19357 | 19391, | 19493, 19527, | 19799, 19833, | 20105, 20139, | | 20411, 20445, |
| | 19425, | 19561, 19595, | 19867, 19901, | 20173, 20207, | | 20479, 20513, |
| | 19459 | 19629, 19663, | 19935, 19969, | 20241, 20275, | | 20547, 20581, |
| | | 19697, 19731, | 20003, 20037, | 20309, 20343, | | 20615, 20649, |
| | | 19765 | 20071 | 20377 | | 20683 |
| 19358 | 19392, | 19494, 19528, | 19800, 19834, | 20106, 20140, | | 20412, 20446, |
| | 19426, | 19562, 19596, | 19868, 19902, | 20174, 20208, | | 20480, 20514, |
| | 19460 | 19630, 19664, | 19936, 19970, | 20242, 20276, | | 20548, 20582, |
| | | 19698, 19732, | 20004, 20038, | 20310, 20344, | | 20616, 20650, |
| | | 19766 | 20072 | 20378 | | 20684 |
| 19359 | 19393, | 19495, 19529, | 19801, 19835, | 20107, 20141, | | 20413, 20447, |
| | 19427, | 19563, 19597, | 19869, 19903, | 20175, 20209, | | 20481, 20515, |
| | 19461 | 19631, 19665, | 19937, 19971, | 20243, 20277, | | 20549, 20583, |
| | | 19699, 19733, | 20005, 20039, | 20311, 20345, | | 20617, 20651, |
| | | 19767 | 20073 | 20379 | | 20685 |
| 19360 | 19394, | 19496, 19530, | 19802, 19836, | 20108, 20142, | | 20414, 20448, |
| | 19428, | 19564, 19598, | 19870, 19904, | 20176, 20210, | | 20482, 20516, |
| | 19462 | 19632, 19666, | 19938, 19972, | 20244, 20278, | | 20550, 20584, |
| | | 19700, 19734, | 20006, 20040, | 20312, 20346, | | 20618, 20652, |
| | | 19768 | 20074 | 20380 | | 20686 |
| 19361 | 19395, | 19497, 19531, | 19803, 19837, | 20109, 20143, | | 20415, 20449, |
| | 19429, | 19565, 19599, | 19871, 19905, | 20177, 20211, | | 20483, 20517, |
| | 19463 | 19633, 19667, | 19939, 19973, | 20245, 20279, | | 20551, 20585, |
| | | 19701, 19735, | 20007, 20041, | 20313, 20347, | | 20619, 20653, |
| | | 19769 | 20075 | 20381 | | 20687 |

TABLE 4.3
Preferred LASV Nucleoprotein (SP-IgE NP) constructs

| | | RNA designs and SEQ ID NOs | | | | |
|---|---|---|---|---|---|---|
| PRT | CDS | a-1 | a-2 | b-4 | i-2 | i-3 |
| 20689 | 20723, | 20825, 20859, | 21131, 21165, | 21437, 21471, | | 21743, 21777, |
| | 20757, | 20893, 20927, | 21199, 21233, | 21505, 21539, | | 21811, 21845, |
| | 20791 | 20961, 20995, | 21267, 21301, | 21573, 21607, | | 21879, 21913, |
| | | 21029, 21063, | 21335, 21369, | 21641, 21675, | | 21947, 21981, |
| | | 21097 | 21403 | 21709 | | 22015 |
| 20690 | 20724, | 20826, 20860, | 21132, 21166, | 21438, 21472, | | 21744, 21778, |
| | 20758, | 20894, 20928, | 21200, 21234, | 21506, 21540, | | 21812, 21846, |
| | 20792 | 20962, 20996, | 21268, 21302, | 21574, 21608, | | 21880, 21914, |
| | | 21030, 21064, | 21336, 21370, | 21642, 21676, | | 21948, 21982, |
| | | 21098 | 21404 | 21710 | | 22016 |
| 20691 | 20725, | 20827, 20861, | 21133, 21167, | 21439, 21473, | | 21745, 21779, |
| | 20759, | 20895, 20929, | 21201, 21235, | 21507, 21541, | | 21813, 21847, |
| | 20793 | 20963, 20997, | 21269, 21303, | 21575, 21609, | | 21881, 21915, |
| | | 21031, 21065, | 21337, 21371, | 21643, 21677, | | 21949, 21983, |
| | | 21099 | 21405 | 21711 | | 22017 |
| 20692 | 20726, | 20828, 20862, | 21134, 21168, | 21440, 21474, | | 21746, 21780, |
| | 20760, | 20896, 20930, | 21202, 21236, | 21508, 21542, | | 21814, 21848, |
| | 20794 | 20964, 20998, | 21270, 21304, | 21576, 21610, | | 21882, 21916, |
| | | 21032, 21066, | 21338, 21372, | 21644, 21678, | | 21950, 21984, |
| | | 21100 | 21406 | 21712 | | 22018 |
| 20693 | 20727, | 20829, 20863, | 21135, 21169, | 21441, 21475, | | 21747, 21781, |
| | 20761, | 20897, 20931, | 21203, 21237, | 21509, 21543, | | 21815, 21849, |
| | 20795 | 20965, 20999, | 21271, 21305, | 21577, 21611, | | 21883, 21917, |
| | | 21033, 21067, | 21339, 21373, | 21645, 21679, | | 21951, 21985, |
| | | 21101 | 21407 | 21713 | | 22019 |
| 20694 | 20728, | 20830, 20864, | 21136, 21170, | 21442, 21476, | | 21748, 21782, |
| | 20762, | 20898, 20932, | 21204, 21238, | 21510, 21544, | | 21816, 21850, |
| | 20796 | 20966, 21000, | 21272, 21306, | 21578, 21612, | | 21884, 21918, |
| | | 21034, 21068, | 21340, 21374, | 21646, 21680, | | 21952, 21986, |
| | | 21102 | 21408 | 21714 | | 22020 |
| 20695 | 20729, | 20831, 20865, | 21137, 21171, | 21443, 21477, | | 21749, 21783, |
| | 20763, | 20899, 20933, | 21205, 21239, | 21511, 21545, | | 21817, 21851, |
| | 20797 | 20967, 21001, | 21273, 21307, | 21579, 21613, | | 21885, 21919, |
| | | 21035, 21069, | 21341, 21375, | 21647, 21681, | | 21953, 21987, |
| | | 21103 | 21409 | 21715 | | 22021 |
| 20696 | 20730, | 20832, 20866, | 21138, 21172, | 21444, 21478, | | 21750, 21784, |
| | 20764, | 20900, 20934, | 21206, 21240, | 21512, 21546, | | 21818, 21852, |
| | 20798 | 20968, 21002, | 21274, 21308, | 21580, 21614, | | 21886, 21920, |
| | | 21036, 21070, | 21342, 21376, | 21648, 21682, | | 21954, 21988, |
| | | 21104 | 21410 | 21716 | | 22022 |
| 20697 | 20731, | 20833, 20867, | 21139, 21173, | 21445, 21479, | | 21751, 21785, |
| | 20765, | 20901, 20935, | 21207, 21241, | 21513, 21547, | | 21819, 21853, |
| | 20799 | 20969, 21003, | 21275, 21309, | 21581, 21615, | | 21887, 21921, |
| | | 21037, 21071, | 21343, 21377, | 21649, 21683, | | 21955, 21989, |
| | | 21105 | 21411 | 21717 | | 22023 |
| 20698 | 20732, | 20834, 20868, | 21140, 21174, | 21446, 21480, | | 21752, 21786, |
| | 20766, | 20902, 20936, | 21208, 21242, | 21514, 21548, | | 21820, 21854, |
| | 20800 | 20970, 21004, | 21276, 21310, | 21582, 21616, | | 21888, 21922, |
| | | 21038, 21072, | 21344, 21378, | 21650, 21684, | | 21956, 21990, |
| | | 21106 | 21412 | 21718 | | 22024 |
| 20699 | 20733, | 20835, 20869, | 21141, 21175, | 21447, 21481, | | 21753, 21787, |
| | 20767, | 20903, 20937, | 21209, 21243, | 21515, 21549, | | 21821, 21855, |
| | 20801 | 20971, 21005, | 21277, 21311, | 21583, 21617, | | 21889, 21923, |
| | | 21039, 21073, | 21345, 21379, | 21651, 21685, | | 21957, 21991, |
| | | 21107 | 21413 | 21719 | | 22025 |
| 20700 | 20734, | 20836, 20870, | 21142, 21176, | 21448, 21482, | | 21754, 21788, |
| | 20768, | 20904, 20938, | 21210, 21244, | 21516, 21550, | | 21822, 21856, |
| | 20802 | 20972, 21006, | 21278, 21312, | 21584, 21618, | | 21890, 21924, |
| | | 21040, 21074, | 21346, 21380, | 21652, 21686, | | 21958, 21992, |
| | | 21108 | 21414 | 21720 | | 22026 |
| 20701 | 20735, | 20837, 20871, | 21143, 21177, | 21449, 21483, | | 21755, 21789, |
| | 20769, | 20905, 20939, | 21211, 21245, | 21517, 21551, | | 21823, 21857, |
| | 20803 | 20973, 21007, | 21279, 21313, | 21585, 21619, | | 21891, 21925, |
| | | 21041, 21075, | 21347, 21381, | 21653, 21687, | | 21959, 21993, |
| | | 21109 | 21415 | 21721 | | 22027 |
| 20702 | 20736, | 20838, 20872, | 21144, 21178, | 21450, 21484, | | 21756, 21790, |
| | 20770, | 20906, 20940, | 21212, 21246, | 21518, 21552, | | 21824, 21858, |
| | 20804 | 20974, 21008, | 21280, 21314, | 21586, 21620, | | 21892, 21926, |
| | | 21042, 21076, | 21348, 21382, | 21654, 21688, | | 21960, 21994, |
| | | 21110 | 21416 | 21722 | | 22028 |
| 20703 | 20737, | 20839, 20873, | 21145, 21179, | 21451, 21485, | | 21757, 21791, |
| | 20771, | 20907, 20941, | 21213, 21247, | 21519, 21553, | | 21825, 21859, |
| | 20805 | 20975, 21009, | 21281, 21315, | 21587, 21621, | | 21893, 21927, |
| | | 21043, 21077, | 21349, 21383, | 21655, 21689, | | 21961, 21995, |
| | | 21111 | 21417 | 21723 | | 22029 |
| 20704 | 20738, | 20840, 20874, | 21146, 21180, | 21452, 21486, | | 21758, 21792, |
| | 20772, | 20908, 20942, | 21214, 21248, | 21520, 21554, | | 21826, 21860, |
| | 20806 | 20976, 21010, | 21282, 21316, | 21588, 21622, | | 21894, 21928, |
| | | 21044, 21078, | 21350, 21384, | 21656, 21690, | | 21962, 21996, |
| | | 21112 | 21418 | 21724 | | 22030 |
| 20705 | 20739, | 20841, 20875, | 21147, 21181, | 21453, 21487, | | 21759, 21793, |
| | 20773, | 20909, 20943, | 21215, 21249, | 21521, 21555, | | 21827, 21861, |
| | 20807 | 20977, 21011, | 21283, 21317, | 21589, 21623, | | 21895, 21929, |
| | | 21045, 21079, | 21351, 21385, | 21657, 21691, | | 21963, 21997, |
| | | 21113 | 21419 | 21725 | | 22031 |
| 20706 | 20740, | 20842, 20876, | 21148, 21182, | 21454, 21488, | | 21760, 21794, |
| | 20774, | 20910, 20944, | 21216, 21250, | 21522, 21556, | | 21828, 21862, |
| | 20808 | 20978, 21012, | 21284, 21318, | 21590, 21624, | | 21896, 21930, |
| | | 21046, 21080, | 21352, 21386, | 21658, 21692, | | 21964, 21998, |
| | | 21114 | 21420 | 21726 | | 22032 |
| 20707 | 20741, | 20843, 20877, | 21149, 21183, | 21455, 21489, | | 21761, 21795, |
| | 20775, | 20911, 20945, | 21217, 21251, | 21523, 21557, | | 21829, 21863, |
| | 20809 | 20979, 21013, | 21285, 21319, | 21591, 21625, | | 21897, 21931, |
| | | 21047, 21081, | 21353, 21387, | 21659, 21693, | | 21965, 21999, |
| | | 21115 | 21421 | 21727 | | 22033 |
| 20708 | 20742, | 20844, 20878, | 21150, 21184, | 21456, 21490, | | 21762, 21796, |
| | 20776, | 20912, 20946, | 21218, 21252, | 21524, 21558, | | 21830, 21864, |
| | 20810 | 20980, 21014, | 21286, 21320, | 21592, 21626, | | 21898, 21932, |
| | | 21048, 21082, | 21354, 21388, | 21660, 21694, | | 21966, 22000, |
| | | 21116 | 21422 | 21728 | | 22034 |
| 20709 | 20743, | 20845, 20879, | 21151, 21185, | 21457, 21491, | | 21763, 21797, |
| | 20777, | 20913, 20947, | 21219, 21253, | 21525, 21559, | | 21831, 21865, |
| | 20811 | 20981, 21015, | 21287, 21321, | 21593, 21627, | | 21899, 21933, |
| | | 21049, 21083, | 21355, 21389, | 21661, 21695, | | 21967, 22001, |
| | | 21117 | 21423 | 21729 | | 22035 |
| 20710 | 20744, | 20846, 20880, | 21152, 21186, | 21458, 21492, | | 21764, 21798, |
| | 20778, | 20914, 20948, | 21220, 21254, | 21526, 21560, | | 21832, 21866, |
| | 20812 | 20982, 21016, | 21288, 21322, | 21594, 21628, | | 21900, 21934, |
| | | 21050, 21084, | 21356, 21390, | 21662, 21696, | | 21968, 22002, |
| | | 21118 | 21424 | 21730 | | 22036 |
| 20711 | 20745, | 20847, 20881, | 21153, 21187, | 21459, 21493, | | 21765, 21799, |
| | 20779, | 20915, 20949, | 21221, 21255, | 21527, 21561, | | 21833, 21867, |
| | 20813 | 20983, 21017, | 21289, 21323, | 21595, 21629, | | 21901, 21935, |
| | | 21051, 21085, | 21357, 21391, | 21663, 21697, | | 21969, 22003, |
| | | 21119 | 21425 | 21731 | | 22037 |
| 20712 | 20746, | 20848, 20882, | 21154, 21188, | 21460, 21494, | | 21766, 21800, |
| | 20780, | 20916, 20950, | 21222, 21256, | 21528, 21562, | | 21834, 21868, |
| | 20814 | 20984, 21018, | 21290, 21324, | 21596, 21630, | | 21902, 21936, |
| | | 21052, 21086, | 21358, 21392, | 21664, 21698, | | 21970, 22004, |
| | | 21120 | 21426 | 21732 | | 22038 |
| 20713 | 20747, | 20849, 20883, | 21155, 21189, | 21461, 21495, | | 21767, 21801, |
| | 20781, | 20917, 20951, | 21223, 21257, | 21529, 21563, | | 21835, 21869, |
| | 20815 | 20985, 21019, | 21291, 21325, | 21597, 21631, | | 21903, 21937, |
| | | 21053, 21087, | 21359, 21393, | 21665, 21699, | | 21971, 22005, |
| | | 21121 | 21427 | 21733 | | 22039 |
| 20714 | 20748, | 20850, 20884, | 21156, 21190, | 21462, 21496, | | 21768, 21802, |
| | 20782, | 20918, 20952, | 21224, 21258, | 21530, 21564, | | 21836, 21870, |
| | 20816 | 20986, 21020, | 21292, 21326, | 21598, 21632, | | 21904, 21938, |
| | | 21054, 21088, | 21360, 21394, | 21666, 21700, | | 21972, 22006, |
| | | 21122 | 21428 | 21734 | | 22040 |
| 20715 | 20749, | 20851, 20885, | 21157, 21191, | 21463, 21497, | | 21769, 21803, |
| | 20783, | 20919, 20953, | 21225, 21259, | 21531, 21565, | | 21837, 21871, |
| | 20817 | 20987, 21021, | 21293, 21327, | 21599, 21633, | | 21905, 21939, |
| | | 21055, 21089, | 21361, 21395, | 21667, 21701, | | 21973, 22007, |
| | | 21123 | 21429 | 21735 | | 22041 |
| 20716 | 20750, | 20852, 20886, | 21158, 21192, | 21464, 21498, | | 21770, 21804, |
| | 20784, | 20920, 20954, | 21226, 21260, | 21532, 21566, | | 21838, 21872, |
| | 20818 | 20988, 21022, | 21294, 21328, | 21600, 21634, | | 21906, 21940, |
| | | 21056, 21090, | 21362, 21396, | 21668, 21702, | | 21974, 22008, |
| | | 21124 | 21430 | 21736 | | 22042 |
| 20717 | 20751, | 20853, 20887, | 21159, 21193, | 21465, 21499, | | 21771, 21805, |
| | 20785, | 20921, 20955, | 21227, 21261, | 21533, 21567, | | 21839, 21873, |
| | 20819 | 20989, 21023, | 21295, 21329, | 21601, 21635, | | 21907, 21941, |
| | | 21057, 21091, | 21363, 21397, | 21669, 21703, | | 21975, 22009, |
| | | 21125 | 21431 | 21737 | | 22043 |
| 20718 | 20752, | 20854, 20888, | 21160, 21194, | 21466, 21500, | | 21772, 21806, |
| | 20786, | 20922, 20956, | 21228, 21262, | 21534, 21568, | | 21840, 21874, |
| | 20820 | 20990, 21024, | 21296, 21330, | 21602, 21636, | | 21908, 21942, |
| | | 21058, 21092, | 21364, 21398, | 21670, 21704, | | 21976, 22010, |
| | | 21126 | 21432 | 21738 | | 22044 |
| 20719 | 20753, | 20855, 20889, | 21161, 21195, | 21467, 21501, | | 21773, 21807, |
| | 20787, | 20923, 20957, | 21229, 21263, | 21535, 21569, | | 21841, 21875, |
| | 20821 | 20991, 21025, | 21297, 21331, | 21603, 21637, | | 21909, 21943, |
| | | 21059, 21093, | 21365, 21399, | 21671, 21705, | | 21977, 22011, |
| | | 21127 | 21433 | 21739 | | 22045 |
| 20720 | 20754, | 20856, 20890, | 21162, 21196, | 21468, 21502, | | 21774, 21808, |
| | 20788, | 20924, 20958, | 21230, 21264, | 21536, 21570, | | 21842, 21876, |
| | 20822 | 20992, 21026, | 21298, 21332, | 21604, 21638, | | 21910, 21944, |
| | | 21060, 21094, | 21366, 21400, | 21672, 21706, | | 21978, 22012, |
| | | 21128 | 21434 | 21740 | | 22046 |
| 20721 | 20755, | 20857, 20891, | 21163, 21197, | 21469, 21503, | | 21775, 21809, |
| | 20789, | 20925, 20959, | 21231, 21265, | 21537, 21571, | | 21843, 21877, |
| | 20823 | 20993, 21027, | 21299, 21333, | 21605, 21639, | | 21911, 21945, |
| | | 21061, 21095, | 21367, 21401, | 21673, 21707, | | 21979, 22013, |
| | | 21129 | 21435 | 21741 | | 22047 |
| 20722 | 20756, | 20858, 20892, | 21164, 21198, | 21470, 21504, | | 21776, 21810, |
| | 20790, | 20926, 20960, | 21232, 21266, | 21538, 21572, | | 21844, 21878, |
| | 20824 | 20994, 21028, | 21300, 21334, | 21606, 21640, | | 21912, 21946, |
| | | 21062, 21096, | 21368, 21402, | 21674, 21708, | | 21980, 22014, |
| | | 21130 | 21436 | 21742 | | 22048 |

TABLE 5
Preferred LASV zinc-binding matrix protein (Z) constructs

| | | RNA designs and SEQ ID NOs | | | | |
|---|---|---|---|---|---|---|
| PRT | CDS | a-1 | a-2 | b-4 | i-2 | i-3 |
| 11166 | 11348, | 12804, 12829, | 13004, 13029, | 13204, 13229, | 13404, | 13604, 13629, |
| | 11530, | 12854, 12879, | 13054, 13079, | 13254, 13279, | 13429, | 13654, 13679, |
| | 11712, | 12904, 12929, | 13104, 13129, | 13304, 13329, | 13454, | 13704, 13729, |
| | 11894, | 12954, 12979, | 13154, 13179, | 13354, 13379, | 13479, | 13754, 13779, |
| | 12076, | 22049, 22074, | 22274, 22299, | 22499, 22524, | 13504, | 22724, 22749, |
| | 12258, | 22099, 22124, | 22324, 22349, | 22549, 22574, | 13529, | 22774, 22799, |
| | 12440, | 22149, 22174, | 22374, 22399, | 22599, 22624, | 13554, | 22824, 22849, |
| | 12622 | 22199, 22224, | 22424, 22449, | 22649, 22674, | 13579 | 22874, 22899, |
| | | 22249 | 22474 | 22699 | | 22924 |
| 11167 | 11349, | 12805, 12830, | 13005, 13030, | 13205, 13230, | 13405, | 13605, 13630, |
| | 11531, | 12855, 12880, | 13055, 13080, | 13255, 13280, | 13430, | 13655, 13680, |
| | 11713, | 12905, 12930, | 13105, 13130, | 13305, 13330, | 13455, | 13705, 13730, |
| | 11895, | 12955, 12980, | 13155, 13180, | 13355, 13380, | 13480, | 13755, 13780, |
| | 12077, | 22050, 22075, | 22275, 22300, | 22500, 22525, | 13505, | 22725, 22750, |
| | 12259, | 22100, 22125, | 22325, 22350, | 22550, 22575, | 13530, | 22775, 22800, |
| | 12441, | 22150, 22175, | 22375, 22400, | 22600, 22625, | 13555, | 22825, 22850, |
| | 12623 | 22200, 22225, | 22425, 22450, | 22650, 22675, | 13580 | 22875, 22900, |
| | | 22250 | 22475 | 22700 | | 22925 |
| 11168 | 11350, | 12806, 12831, | 13006, 13031, | 13206, 13231, | 13406, | 13606, 13631, |
| | 11532, | 12856, 12881, | 13056, 13081, | 13256, 13281, | 13431, | 13656, 13681, |
| | 11714, | 12906, 12931, | 13106, 13131, | 13306, 13331, | 13456, | 13706, 13731, |
| | 11896, | 12956, 12981, | 13156, 13181, | 13356, 13381, | 13481, | 13756, 13781, |
| | 12078, | 22051, 22076, | 22276, 22301, | 22501, 22526, | 13506, | 22726, 22751, |
| | 12260, | 22101, 22126, | 22326, 22351, | 22551, 22576, | 13531, | 22776, 22801, |
| | 12442, | 22151, 22176, | 22376, 22401, | 22601, 22626, | 13556, | 22826, 22851, |
| | 12624 | 22201, 22226, | 22426, 22451, | 22651, 22676, | 13581 | 22876, 22901, |
| | | 22251 | 22476 | 22701 | | 22926 |
| 11169 | 11351, | 12807, 12832, | 13007, 13032, | 13207, 13232, | 13407, | 13607, 13632, |
| | 11533, | 12857, 12882, | 13057, 13082, | 13257, 13282, | 13432, | 13657, 13682, |
| | 11715, | 12907, 12932, | 13107, 13132, | 13307, 13332, | 13457, | 13707, 13732, |
| | 11897, | 12957, 12982, | 13157, 13182, | 13357, 13382, | 13482, | 13757, 13782, |
| | 12079, | 22052, 22077, | 22277, 22302, | 22502, 22527, | 13507, | 22727, 22752, |
| | 12261, | 22102, 22127, | 22327, 22352, | 22552, 22577, | 13532, | 22777, 22802, |
| | 12443, | 22152, 22177, | 22377, 22402, | 22602, 22627, | 13557, | 22827, 22852, |
| | 12625 | 22202, 22227, | 22427, 22452, | 22652, 22677, | 13582 | 22877, 22902, |
| | | 22252 | 22477 | 22702 | | 22927 |
| 11170 | 11352, | 12808, 12833, | 13008, 13033, | 13208, 13233, | 13408, | 13608, 13633, |
| | 11534, | 12858, 12883, | 13058, 13083, | 13258, 13283, | 13433, | 13658, 13683, |
| | 11716, | 12908, 12933, | 13108, 13133, | 13308, 13333, | 13458, | 13708, 13733, |
| | 11898, | 12958, 12983, | 13158, 13183, | 13358, 13383, | 13483, | 13758, 13783, |
| | 12080, | 22053, 22078, | 22278, 22303, | 22503, 22528, | 13508, | 22728, 22753, |
| | 12262, | 22103, 22128, | 22328, 22353, | 22553, 22578, | 13533, | 22778, 22803, |
| | 12444, | 22153, 22178, | 22378, 22403, | 22603, 22628, | 13558, | 22828, 22853, |
| | 12626 | 22203, 22228, | 22428, 22453, | 22653, 22678, | 13583 | 22878, 22903, |
| | | 22253 | 22478 | 22703 | | 22928 |
| 11171 | 11353, | 12809, 12834, | 13009, 13034, | 13209, 13234, | 13409, | 13609, 13634, |
| | 11535, | 12859, 12884, | 13059, 13084, | 13259, 13284, | 13434, | 13659, 13684, |
| | 11717, | 12909, 12934, | 13109, 13134, | 13309, 13334, | 13459, | 13709, 13734, |
| | 11899, | 12959, 12984, | 13159, 13184, | 13359, 13384, | 13484, | 13759, 13784, |
| | 12081, | 22054, 22079, | 22279, 22304, | 22504, 22529, | 13509, | 22729, 22754, |
| | 12263, | 22104, 22129, | 22329, 22354, | 22554, 22579, | 13534, | 22779, 22804, |
| | 12445, | 22154, 22179, | 22379, 22404, | 22604, 22629, | 13559, | 22829, 22854, |
| | 12627 | 22204, 22229, | 22429, 22454, | 22654, 22679, | 13584 | 22879, 22904, |
| | | 22254 | 22479 | 22704 | | 22929 |
| 11172 | 11354, | 12810, 12835, | 13010, 13035, | 13210, 13235, | 13410, | 13610, 13635, |
| | 11536, | 12860, 12885, | 13060, 13085, | 13260, 13285, | 13435, | 13660, 13685, |
| | 11718, | 12910, 12935, | 13110, 13135, | 13310, 13335, | 13460, | 13710, 13735, |
| | 11900, | 12960, 12985, | 13160, 13185, | 13360, 13385, | 13485, | 13760, 13785, |
| | 12082, | 22055, 22080, | 22280, 22305, | 22505, 22530, | 13510, | 22730, 22755, |
| | 12264, | 22105, 22130, | 22330, 22355, | 22555, 22580, | 13535, | 22780, 22805, |
| | 12446, | 22155, 22180, | 22380, 22405, | 22605, 22630, | 13560, | 22830, 22855, |
| | 12628 | 22205, 22230, | 22430, 22455, | 22655, 22680, | 13585 | 22880, 22905, |
| | | 22255 | 22480 | 22705 | | 22930 |
| 11173 | 11355, | 12811, 12836, | 13011, 13036, | 13211, 13236, | 13411, | 13611, 13636, |
| | 11537, | 12861, 12886, | 13061, 13086, | 13261, 13286, | 13436, | 13661, 13686, |
| | 11719, | 12911, 12936, | 13111, 13136, | 13311, 13336, | 13461, | 13711, 13736, |
| | 11901, | 12961, 12986, | 13161, 13186, | 13361, 13386, | 13486, | 13761, 13786, |
| | 12083, | 22056, 22081, | 22281, 22306, | 22506, 22531, | 13511, | 22731, 22756, |
| | 12265, | 22106, 22131, | 22331, 22356, | 22556, 22581, | 13536, | 22781, 22806, |
| | 12447, | 22156, 22181, | 22381, 22406, | 22606, 22631, | 13561, | 22831, 22856, |
| | 12629 | 22206, 22231, | 22431, 22456, | 22656, 22681, | 13586 | 22881, 22906, |
| | | 22256 | 22481 | 22706 | | 22931 |
| 11190 | 11372, | 12812, 12837, | 13012, 13037, | 13212, 13237, | 13412, | 13612, 13637, |
| | 11554, | 12862, 12887, | 13062, 13087, | 13262, 13287, | 13437, | 13662, 13687, |
| | 11736, | 12912, 12937, | 13112, 13137, | 13312, 13337, | 13462, | 13712, 13737, |
| | 11918, | 12962, 12987, | 13162, 13187, | 13362, 13387, | 13487, | 13762, 13787, |
| | 12100, | 22057, 22082, | 22282, 22307, | 22507, 22532, | 13512, | 22732, 22757, |
| | 12282, | 22107, 22132, | 22332, 22357, | 22557, 22582, | 13537, | 22782, 22807, |
| | 12464, | 22157, 22182, | 22382, 22407, | 22607, 22632, | 13562, | 22832, 22857, |
| | 12646 | 22207, 22232, | 22432, 22457, | 22657, 22682, | 13587 | 22882, 22907, |
| | | 22257 | 22482 | 22707 | | 22932 |
| 11208 | 11390, | 12813, 12838, | 13013, 13038, | 13213, 13238, | 13413, | 13613, 13638, |
| | 11572, | 12863, 12888, | 13063, 13088, | 13263, 13288, | 13438, | 13663, 13688, |
| | 11754, | 12913, 12938, | 13113, 13138, | 13313, 13338, | 13463, | 13713, 13738, |
| | 11936, | 12963, 12988, | 13163, 13188, | 13363, 13388, | 13488, | 13763, 13788, |
| | 12118, | 22058, 22083, | 22283, 22308, | 22508, 22533, | 13513, | 22733, 22758, |
| | 12300, | 22108, 22133, | 22333, 22358, | 22558, 22583, | 13538, | 22783, 22808, |
| | 12482, | 22158, 22183, | 22383, 22408, | 22608, 22633, | 13563, | 22833, 22858, |
| | 12664 | 22208, 22233, | 22433, 22458, | 22658, 22683, | 13588 | 22883, 22908, |
| | | 22258 | 22483 | 22708 | | 22933 |
| 11229 | 11411, | 12814, 12839, | 13014, 13039, | 13214, 13239, | 13414, | 13614, 13639, |
| | 11593, | 12864, 12889, | 13064, 13089, | 13264, 13289, | 13439, | 13664, 13689, |
| | 11775, | 12914, 12939, | 13114, 13139, | 13314, 13339, | 13464, | 13714, 13739, |
| | 11957, | 12964, 12989, | 13164, 13189, | 13364, 13389, | 13489, | 13764, 13789, |
| | 12139, | 22059, 22084, | 22284, 22309, | 22509, 22534, | 13514, | 22734, 22759, |
| | 12321, | 22109, 22134, | 22334, 22359, | 22559, 22584, | 13539, | 22784, 22809, |
| | 12503, | 22159, 22184, | 22384, 22409, | 22609, 22634, | 13564, | 22834, 22859, |
| | 12685 | 22209, 22234, | 22434, 22459, | 22659, 22684, | 13589 | 22884, 22909, |
| | | 22259 | 22484 | 22709 | | 22934 |
| 11233 | 11415, | 12815, 12840, | 13015, 13040, | 13215, 13240, | 13415, | 13615, 13640, |
| | 11597, | 12865, 12890, | 13065, 13090, | 13265, 13290, | 13440, | 13665, 13690, |
| | 11779, | 12915, 12940, | 13115, 13140, | 13315, 13340, | 13465, | 13715, 13740, |
| | 11961, | 12965, 12990, | 13165, 13190, | 13365, 13390, | 13490, | 13765, 13790, |
| | 12143, | 22060, 22085, | 22285, 22310, | 22510, 22535, | 13515, | 22735, 22760, |
| | 12325, | 22110, 22135, | 22335, 22360, | 22560, 22585, | 13540, | 22785, 22810, |
| | 12507, | 22160, 22185, | 22385, 22410, | 22610, 22635, | 13565, | 22835, 22860, |
| | 12689 | 22210, 22235, | 22435, 22460, | 22660, 22685, | 13590 | 22885, 22910, |
| | | 22260 | 22485 | 22710 | | 22935 |
| 11234 | 11416, | 12816, 12841, | 13016, 13041, | 13216, 13241, | 13416, | 13616, 13641, |
| | 11598, | 12866, 12891, | 13066, 13091, | 13266, 13291, | 13441, | 13666, 13691, |
| | 11780, | 12916, 12941, | 13116, 13141, | 13316, 13341, | 13466, | 13716, 13741, |
| | 11962, | 12966, 12991, | 13166, 13191, | 13366, 13391, | 13491, | 13766, 13791, |
| | 12144, | 22061, 22086, | 22286, 22311, | 22511, 22536, | 13516, | 22736, 22761, |
| | 12326, | 22111, 22136, | 22336, 22361, | 22561, 22586, | 13541, | 22786, 22811, |
| | 12508, | 22161, 22186, | 22386, 22411, | 22611, 22636, | 13566, | 22836, 22861, |
| | 12690 | 22211, 22236, | 22436, 22461, | 22661, 22686, | 13591 | 22886, 22911, |
| | | 22261 | 22486 | 22711 | | 22936 |
| 11240 | 11422, | 12817, 12842, | 13017, 13042, | 13217, 13242, | 13417, | 13617, 13642, |
| | 11604, | 12867, 12892, | 13067, 13092, | 13267, 13292, | 13442, | 13667, 13692, |
| | 11786, | 12917, 12942, | 13117, 13142, | 13317, 13342, | 13467, | 13717, 13742, |
| | 11968, | 12967, 12992, | 13167, 13192, | 13367, 13392, | 13492, | 13767, 13792, |
| | 12150, | 22062, 22087, | 22287, 22312, | 22512, 22537, | 13517, | 22737, 22762, |
| | 12332, | 22112, 22137, | 22337, 22362, | 22562, 22587, | 13542, | 22787, 22812, |
| | 12514, | 22162, 22187, | 22387, 22412, | 22612, 22637, | 13567, | 22837, 22862, |
| | 12696 | 22212, 22237, | 22437, 22462, | 22662, 22687, | 13592 | 22887, 22912, |
| | | 22262 | 22487 | 22712 | | 22937 |
| 11245 | 11427, | 12818, 12843, | 13018, 13043, | 13218, 13243, | 13418, | 13618, 13643, |
| | 11609, | 12868, 12893, | 13068, 13093, | 13268, 13293, | 13443, | 13668, 13693, |
| | 11791, | 12918, 12943, | 13118, 13143, | 13318, 13343, | 13468, | 13718, 13743, |
| | 11973, | 12968, 12993, | 13168, 13193, | 13368, 13393, | 13493, | 13768, 13793, |
| | 12155, | 22063, 22088, | 22288, 22313, | 22513, 22538, | 13518, | 22738, 22763, |
| | 12337, | 22113, 22138, | 22338, 22363, | 22563, 22588, | 13543, | 22788, 22813, |
| | 12519, | 22163, 22188, | 22388, 22413, | 22613, 22638, | 13568, | 22838, 22863, |
| | 12701 | 22213, 22238, | 22438, 22463, | 22663, 22688, | 13593 | 22888, 22913, |
| | | 22263 | 22488 | 22713 | | 22938 |
| 11248 | 11430, | 12819, 12844, | 13019, 13044, | 13219, 13244, | 13419, | 13619, 13644, |
| | 11612, | 12869, 12894, | 13069, 13094, | 13269, 13294, | 13444, | 13669, 13694, |
| | 11794, | 12919, 12944, | 13119, 13144, | 13319, 13344, | 13469, | 13719, 13744, |
| | 11976, | 12969, 12994, | 13169, 13194, | 13369, 13394, | 13494, | 13769, 13794, |
| | 12158, | 22064, 22089, | 22289, 22314, | 22514, 22539, | 13519, | 22739, 22764, |
| | 12340, | 22114, 22139, | 22339, 22364, | 22564, 22589, | 13544, | 22789, 22814, |
| | 12522, | 22164, 22189, | 22389, 22414, | 22614, 22639, | 13569, | 22839, 22864, |
| | 12704 | 22214, 22239, | 22439, 22464, | 22664, 22689, | 13594 | 22889, 22914, |
| | | 22264 | 22489 | 22714 | | 22939 |
| 11255 | 11437, | 12820, 12845, | 13020, 13045, | 13220, 13245, | 13420, | 13620, 13645, |
| | 11619, | 12870, 12895, | 13070, 13095, | 13270, 13295, | 13445, | 13670, 13695, |
| | 11801, | 12920, 12945, | 13120, 13145, | 13320, 13345, | 13470, | 13720, 13745, |
| | 11983, | 12970, 12995, | 13170, 13195, | 13370, 13395, | 13495, | 13770, 13795, |
| | 12165, | 22065, 22090, | 22290, 22315, | 22515, 22540, | 13520, | 22740, 22765, |
| | 12347, | 22115, 22140, | 22340, 22365, | 22565, 22590, | 13545, | 22790, 22815, |
| | 12529, | 22165, 22190, | 22390, 22415, | 22615, 22640, | 13570, | 22840, 22865, |
| | 12711 | 22215, 22240, | 22440, 22465, | 22665, 22690, | 13595 | 22890, 22915, |
| | | 22265 | 22490 | 22715 | | 22940 |
| 11278 | 11460, | 12821, 12846, | 13021, 13046, | 13221, 13246, | 13421, | 13621, 13646, |
| | 11642, | 12871, 12896, | 13071, 13096, | 13271, 13296, | 13446, | 13671, 13696, |
| | 11824, | 12921, 12946, | 13121, 13146, | 13321, 13346, | 13471, | 13721, 13746, |
| | 12006, | 12971, 12996, | 13171, 13196, | 13371, 13396, | 13496, | 13771, 13796, |
| | 12188, | 22066, 22091, | 22291, 22316, | 22516, 22541, | 13521, | 22741, 22766, |
| | 12370, | 22116, 22141, | 22341, 22366, | 22566, 22591, | 13546, | 22791, 22816, |
| | 12552, | 22166, 22191, | 22391, 22416, | 22616, 22641, | 13571, | 22841, 22866, |
| | 12734 | 22216, 22241, | 22441, 22466, | 22666, 22691, | 13596 | 22891, 22916, |
| | | 22266 | 22491 | 22716 | | 22941 |
| 11281 | 11463, | 12822, 12847, | 13022, 13047, | 13222, 13247, | 13422, | 13622, 13647, |
| | 11645, | 12872, 12897, | 13072, 13097, | 13272, 13297, | 13447, | 13672, 13697, |
| | 11827, | 12922, 12947, | 13122, 13147, | 13322, 13347, | 13472, | 13722, 13747, |
| | 12009, | 12972, 12997, | 13172, 13197, | 13372, 13397, | 13497, | 13772, 13797, |
| | 12191, | 22067, 22092, | 22292, 22317, | 22517, 22542, | 13522, | 22742, 22767, |
| | 12373, | 22117, 22142, | 22342, 22367, | 22567, 22592, | 13547, | 22792, 22817, |
| | 12555, | 22167, 22192, | 22392, 22417, | 22617, 22642, | 13572, | 22842, 22867, |
| | 12737 | 22217, 22242, | 22442, 22467, | 22667, 22692, | 13597 | 22892, 22917, |
| | | 22267 | 22492 | 22717 | | 22942 |
| 11321 | 11503, | 12823, 12848, | 13023, 13048, | 13223, 13248, | 13423, | 13623, 13648, |
| | 11685, | 12873, 12898, | 13073, 13098, | 13273, 13298, | 13448, | 13673, 13698, |
| | 11867, | 12923, 12948, | 13123, 13148, | 13323, 13348, | 13473, | 13723, 13748, |
| | 12049, | 12973, 12998, | 13173, 13198, | 13373, 13398, | 13498, | 13773, 13798, |
| | 12231, | 22068, 22093, | 22293, 22318, | 22518, 22543, | 13523, | 22743, 22768, |
| | 12413, | 22118, 22143, | 22343, 22368, | 22568, 22593, | 13548, | 22793, 22818, |
| | 12595, | 22168, 22193, | 22393, 22418, | 22618, 22643, | 13573, | 22843, 22868, |
| | 12777 | 22218, 22243, | 22443, 22468, | 22668, 22693, | 13598 | 22893, 22918, |
| | | 22268 | 22493 | 22718 | | 22943 |
| 11326 | 11508, | 12824, 12849, | 13024, 13049, | 13224, 13249, | 13424, | 13624, 13649, |
| | 11690, | 12874, 12899, | 13074, 13099, | 13274, 13299, | 13449, | 13674, 13699, |
| | 11872, | 12924, 12949, | 13124, 13149, | 13324, 13349, | 13474, | 13724, 13749, |
| | 12054, | 12974, 12999, | 13174, 13199, | 13374, 13399, | 13499, | 13774, 13799, |
| | 12236, | 22069, 22094, | 22294, 22319, | 22519, 22544, | 13524, | 22744, 22769, |
| | 12418, | 22119, 22144, | 22344, 22369, | 22569, 22594, | 13549, | 22794, 22819, |
| | 12600, | 22169, 22194, | 22394, 22419, | 22619, 22644, | 13574, | 22844, 22869, |
| | 12782 | 22219, 22244, | 22444, 22469, | 22669, 22694, | 13599 | 22894, 22919, |
| | | 22269 | 22494 | 22719 | | 22944 |
| 11330 | 11512, | 12825, 12850, | 13025, 13050, | 13225, 13250, | 13425, | 13625, 13650, |
| | 11694, | 12875, 12900, | 13075, 13100, | 13275, 13300, | 13450, | 13675, 13700, |
| | 11876, | 12925, 12950, | 13125, 13150, | 13325, 13350, | 13475, | 13725, 13750, |
| | 12058, | 12975, 13000, | 13175, 13200, | 13375, 13400, | 13500, | 13775, 13800, |
| | 12240, | 22070, 22095, | 22295, 22320, | 22520, 22545, | 13525, | 22745, 22770, |
| | 12422, | 22120, 22145, | 22345, 22370, | 22570, 22595, | 13550, | 22795, 22820, |
| | 12604, | 22170, 22195, | 22395, 22420, | 22620, 22645, | 13575, | 22845, 22870, |
| | 12786 | 22220, 22245, | 22445, 22470, | 22670, 22695, | 13600 | 22895, 22920, |
| | | 22270 | 22495 | 22720 | | 22945 |
| 11334 | 11516, | 12826, 12851, | 13026, 13051, | 13226, 13251, | 13426, | 13626, 13651, |
| | 11698, | 12876, 12901, | 13076, 13101, | 13276, 13301, | 13451, | 13676, 13701, |
| | 11880, | 12926, 12951, | 13126, 13151, | 13326, 13351, | 13476, | 13726, 13751, |
| | 12062, | 12976, 13001, | 13176, 13201, | 13376, 13401, | 13501, | 13776, 13801, |
| | 12244, | 22071, 22096, | 22296, 22321, | 22521, 22546, | 13526, | 22746, 22771, |
| | 12426, | 22121, 22146, | 22346, 22371, | 22571, 22596, | 13551, | 22796, 22821, |
| | 12608, | 22171, 22196, | 22396, 22421, | 22621, 22646, | 13576, | 22846, 22871, |
| | 12790 | 22221, 22246, | 22446, 22471, | 22671, 22696, | 13601 | 22896, 22921, |
| | | 22271 | 22496 | 22721 | | 22946 |
| 11335 | 11517, | 12827, 12852, | 13027, 13052, | 13227, 13252, | 13427, | 13627, 13652, |
| | 11699, | 12877, 12902, | 13077, 13102, | 13277, 13302, | 13452, | 13677, 13702, |
| | 11881, | 12927, 12952, | 13127, 13152, | 13327, 13352, | 13477, | 13727, 13752, |
| | 12063, | 12977, 13002, | 13177, 13202, | 13377, 13402, | 13502, | 13777, 13802, |
| | 12245, | 22072, 22097, | 22297, 22322, | 22522, 22547, | 13527, | 22747, 22772, |
| | 12427, | 22122, 22147, | 22347, 22372, | 22572, 22597, | 13552, | 22797, 22822, |
| | 12609, | 22172, 22197, | 22397, 22422, | 22622, 22647, | 13577, | 22847, 22872, |
| | 12791 | 22222, 22247, | 22447, 22472, | 22672, 22697, | 13602 | 22897, 22922, |
| | | 22272 | 22497 | 22722 | | 22947 |
| 11341 | 11523, | 12828, 12853, | 13028, 13053, | 13228, 13253, | 13428, | 13628, 13653, |
| | 11705, | 12878, 12903, | 13078, 13103, | 13278, 13303, | 13453, | 13678, 13703, |
| | 11887, | 12928, 12953, | 13128, 13153, | 13328, 13353, | 13478, | 13728, 13753, |
| | 12069, | 12978, 13003, | 13178, 13203, | 13378, 13403, | 13503, | 13778, 13803, |
| | 12251, | 22073, 22098, | 22298, 22323, | 22523, 22548, | 13528, | 22748, 22773, |
| | 12433, | 22123, 22148, | 22348, 22373, | 22573, 22598, | 13553, | 22798, 22823, |
| | 12615, | 22173, 22198, | 22398, 22423, | 22623, 22648, | 13578, | 22848, 22873, |
| | 12797 | 22223, 22248, | 22448, 22473, | 22673, 22698, | 13603 | 22898, 22923, |
| | | 22273 | 22498 | 22723 | | 22948 |

In preferred embodiments, the RNA of the first aspect comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2287- 3566, 6107-7546, 9806-11165, 12804-13803, 14056-17967, 18104-19327, 19464-20687, 20825-22948, 23109-24644, 24805-26340, 26501-28036, 28197-29732, 29893-31428, 31589-33124, 33285-34820, 34981-36516, 36677-38212, 38373-39908, 41765-43300 or a fragment or variant of any of these sequences. Further information e.g. regarding the type of coding sequence (wt, opt1, opt2, opt3, opt4, opt5, opt6, opt11 etc.) comprised in the RNA constructs, the antigen, or the UTR sequences are provided under <223> identifier of the respective SEQ ID NO in the sequence listing.

In particularly preferred embodiments, the RNA of the first aspect comprises
(a) at least one heterologous 5′ untranslated region (5′-UTR) and/or at least one heterologous 3′ untranslated region (3′-UTR) and
(b) at least one coding sequence operably linked to said 3′-UTR and/or 5′-UTR encoding at least one antigenic peptide or protein derived from a LASV protein or a fragment or variant thereof, wherein
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2287-3566, 14056-15207 (encoding GPC) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 6107-7546, 15208-16743 (encoding GPCmut1) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 23109-24644 (encoding GPCmut2) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 24805-26340 (encoding GPCmut3) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 26501-28036 (encoding GPCmut4) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 28197-29732 (encoding GPCmut5) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 29893-31428 (encoding GPCmut6) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 31589-33124 (encoding GPCmut7) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 33285-34820 (encoding GPCmut8) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 34981-36516 (encoding GPCmut9) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 36677-38212 (encoding GPCmut10) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 38373-39908 (encoding GPCmut11) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 40069-41604 (encoding GPCmut12) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 41765-43300 (encoding GPCmut13) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 9806-11165, 16744-17967 (encoding NP) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 18104-19327, 19464-20687, 20825-22048 (encoding SP-NP) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 12804-13803, 22049-22948 (encoding Z) or a fragment or variant of any of these sequences.

As outlined throughout the specification, additional information regarding suitable amino acid sequences or suitable nucleic acid sequences (coding sequences, mRNA sequences) may also be derived from the sequence listing, in particular from the details provided therein under identifier <223> as explained in the following. Therein, the skilled person can easily obtain additional information for each sequence type (amino acid sequence, nucleic acid sequence, e.g. coding sequence or mRNA sequence).

It has to be noted that throughout the sequence listing, information provided under numeric identifier <223> follows the same structure: “<SEQUENCE_DESCRIPTOR> from <CONSTRUCT_IDENTIFIER>”. The <SEQUENCE_DESCRIPTOR> relates to the type of sequence (e.g., “derived and/or modified protein sequence”, “derived and/or modified CDS sequence” “mRNA product Design a-1 comprising derived and/or modified sequence”, or “mRNA product Design a-2 comprising derived and/or modified sequence”, or “mRNA product Design i-2 comprising derived and/or modified sequence”, etc.) and whether the (coding) sequence comprises or consists of a wild type sequence (“wt”) or whether the sequence comprises or consists of a sequence-optimized sequence (e.g. “opt1”, “opt2”, “opt3”, “opt4”, “opt5”, “opt6”, “opt11”; sequence optimizations are described in further detail below).

For example, the <SEQUENCE_DESCRIPTOR> provided under numeric identifier <223> of SEQ ID NO: 8 reads as follows: “derived and/or modified protein sequence (wt)”. The <CONSTRUCT_IDENTIFIER> provided under numeric identifier <223> has the following structures: (“LASV(strain; clade)_construct name”, or “organism_accession number_construct name”) and is intended to help the person skilled in the art to explicitly derive suitable nucleic acid sequences (e.g., RNA, mRNA) encoding the same LASV protein according to the invention. For example, the <CONSTRUCT_IDENTIFIER> provided under numeric identifier <223> of SEQ ID NO: 8 reads as follows: “LASV(Acar 3080;Clade I)_AAT49014_GPC”. In that example, the respective protein sequence is derived from LASV Acar 3080, a member of Clade I, wherein the amino acid sequence comprises GPC. Moreover, the NCBI accession number is indicated “AAT49014”. If the skilled person uses the construct identifier of SEQ ID NO: 8, namely “LASV(Acar 3080;Clade I)_AAT49014_GPC” or the NCBI accession number “AAT49014” said person easily arrive at a list of suitable nucleic acid coding sequences, e.g. RNA coding sequences and mRNA sequences encoding said respective LASV antigenic protein, that can easily be retrieve from the sequence listing of the present invention.

Accordingly, the sequence listing of the invention provides additional information (under <223> identifier) that is explicitly included herein as part of the description. Moreover, said additional information enables the skilled person to obtain suitable sequences encoding the same antigenic peptide or protein.

Composition:

A second aspect relates to a composition comprising at least one RNA of the first aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect (comprising the RNA of the first aspect).

In preferred embodiments, said composition comprises at least one coding RNA encoding a LASV antigenic peptide or protein, preferably GPC or prefusion-stabilized GPC according to the first aspect, or an immunogenic fragment or immunogenic variant thereof, wherein said composition is to be, preferably, administered intramuscularly or intradermal.

Preferably, intramuscular or intradermal administration of said composition results in expression of the encoded LASV antigen construct in a subject. Preferably, the composition of the second aspect is suitable for a vaccine, in particular, suitable for a LASV vaccine.

In the context of the invention, a “composition” refers to any type of composition in which the specified ingredients (e.g. RNA encoding GPC e.g. in association with a polymeric carrier or LNP), may be incorporated, optionally along with any further constituents, usually with at least one pharmaceutically acceptable carrier or excipient. The composition may be a dry composition such as a powder or granules, or a solid unit such as a lyophilized form. Alternatively, the composition may be in liquid form, and each constituent may be independently incorporated in dissolved or dispersed (e.g. suspended or emulsified) form.

In a preferred embodiment of the second aspect, the composition comprises at least one RNA of the first aspect and, optionally, at least one pharmaceutically acceptable carrier.

In particularly preferred embodiments of the second aspect, the composition comprises at least one RNA, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs 255-2286, 3821-6106, 7798-9805, 11348-12803, 18002-18103, 19362-19463, 20723-20824, 22981-23108, 24677-24804, 26373-26500, 28069-28196, 29765-29892, 31461-31588, 33157-33284, 34853-34980, 36549-36676, 38245-38372, 41637-41764, 14056-17967, 18104-19327, 19464-20687, 20825-22948, 23109-24644, 24805-26340, 26501-28036, 28197-29732, 29893-31428, 31589-33124, 33285-34820, 34981-36516, 36677-38212, 38373-39908, 41765-43300 and, optionally, at least one pharmaceutically acceptable carrier or excipient.

The term “pharmaceutically acceptable carrier” as used herein preferably includes the liquid or non-liquid basis of the composition. If the composition is provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, Kl, KBr, K₂CO₃, KHCO₃, K₂SO₄, and examples of calcium salts include CaCl₂, CaI₂, CaBr₂, CaCO₃, CaSO₄, Ca(OH)₂. Furthermore, organic anions of the aforementioned cations may be in the buffer.

In preferred embodiments, the composition comprises more than one or a plurality, e.g. 2, 3, 4, 5, 6, 7, 8, 9, 10, 11, 12, 13, 14, 15 of different RNAs each defined in the first aspect of the invention.

In embodiments, the at least one RNA comprised in the composition is a bi- or multicistronic nucleic acid, particularly a bi- or multicistronic nucleic acid as defined herein, which encodes the at least two, three, four, five, six, seven, eight, nine, ten, eleven or twelve distinct antigenic peptides or protein derived from the same LASV and/or a different LASV.

In embodiment, the composition may comprise at least 2, 3, 4, 5, 6, 7, 8, 9, 10 or even more different RNAs as defined in the context of the first aspect each encoding at least one antigenic peptide or protein derived from genetically the same LASV or a fragment or variant thereof. The term “same LASV” as used in the context of a virus, e.g. “same virus”, has to be understood as genetically the same. Particularly, said (genetically) same virus expresses essentially the same proteins or peptides, wherein all proteins or peptides have essentially the same amino acid sequence.

In embodiments, the composition may comprise at least 2, 3, 4, 5, 6, 7, 8, 9, 10 or even more different RNAs as defined in the context of the first aspect each encoding at least one antigenic peptide or protein derived from a genetically different LASV or a fragment or variant thereof. The term “different LASV” as used in the context of a virus, e.g. “different virus”, has to be understood as the difference manifested on the RNA genome of the respective different virus. Particularly, said (genetically) different LASV expresses at least one different protein, peptide or polyprotein, wherein the at least one different protein, peptide or polyprotein preferably differs in at least one amino acid.

In preferred embodiments, the composition of the second aspect may comprise at least 2, 3, 4, 5, 6, 7, 8, 9, 10 or even more different RNAs, wherein each of said RNAs encodes the same antigenic peptide or protein derived from GPC or prefusion-stabilized GPC of different LASV, preferably derived of different LASV clades.

In preferred embodiments, the composition of the second aspect comprises
(i) at least one coding RNA encoding at least one antigenic protein that is or is derived from GPC or prefusion-stabilized GPC of a LASV strain from clade I as specified herein; and
(ii) at least one coding RNA encoding at least one antigenic protein that is or is derived from GPC or prefusion-stabilized GPC of a LASV strain from clade II as specified herein; and
(iii) at least one coding RNA encoding at least one antigenic protein that is or is derived from GPC or prefusion-stabilized GPC of a LASV strain from clade III as specified herein; and
(iv) at least one coding RNA encoding at least one antigenic protein that is or is derived from GPC or prefusion-stabilized GPC of a LASV strain from clade IV as specified herein.

In particularly preferred embodiments, the composition of the second aspect comprises
(i) at least one coding RNA encoding at least one antigenic protein that is or is derived from GPC or prefusion-stabilized GPC of a LASV strain from clade I, preferably of strain LP,
(ii) at least one coding RNA encoding at least one antigenic protein that is or is derived from GPC or prefusion-stabilized GPC of a LASV strain from clade II, preferably of strain 803213 and
(iii) at least one coding RNA encoding at least one antigenic protein that is or is derived from GPC or prefusion-stabilized GPC of a LASV strain from clade III, preferably of strain GA391; and
(iv) at least one coding RNA encoding at least one antigenic protein that is or is derived from GPC or prefusion-stabilized GPC of a LASV strain from clade IV preferably of strain Josiah.

In particularly preferred embodiments, the composition of the second aspect comprises
(i) at least one coding RNA encoding at least one antigenic protein that is or is derived from GPC or prefusion-stabilized GPC of a LASV strain from clade I, preferably of strain LP, preferably encoding a protein according to SEQ ID NO: 2, 3568, 22950, 24646, 26342, 28038, 29734, 31430, 33126, 34822, 36518, 38214, 39910, 41606, 7548, 17969, 19329, 20690,
(ii) at least one coding RNA encoding at least one antigenic protein that is or is derived from GPC or prefusion-stabilized GPC of a LASV strain from clade II, preferably of strain 803213, preferably encoding a protein according to SEQ ID NO: 3, 3569, 22951, 24647, 26343, 28039, 29735, 31431, 33127, 34823, 36519, 38215, 39911, 41607, 7549, 17970, 19330, 20691,
(iii) at least one coding RNA encoding at least one antigenic protein that is or is derived from GPC or prefusion-stabilized GPC of a LASV strain from clade III, preferably of strain GA391, preferably encoding a protein according to SEQ ID NO: 4, 3570, 22952, 24648, 26344, 28040, 29736, 31432, 33128, 34824, 36520, 38216, 39912, 41608, 7550, 7692, 17971, 17990, 19331, 19350, 20692, 20711, and
(iv) at least one coding RNA encoding at least one antigenic protein that is or is derived from GPC or prefusion-stabilized GPC of a LASV strain from clade IV preferably of strain Josiah, preferably encoding a protein according to SEQ ID NO: 1, 3567, 22949, 24645, 26341, 28037, 29733, 31429, 33125, 34821, 36517, 38213, 39909, 41605, 7547, 17968, 19328, 20689, 11166.

In further preferred embodiments, the composition of the second aspect comprises
(i) at least one coding RNA encoding at least one antigenic protein that is or is derived from GPC or prefusion-stabilized GPC of a LASV strain from clade I, preferably of strain LP,
(ii) at least one coding RNA encoding at least one antigenic protein that is or is derived from GPC or prefusion-stabilized GPC of a LASV strain from clade II, preferably of strain 803213 ISTH0009-NIG-2011, ISTH2010-NIG-2012, Lassa virus/H.sapiens-wt/NGA/2015/ISTH_0543, Lassa virus/H.sapiens-wt/NGA/2016/ISTH_0009, Lassa virus/H.sapiens-wt/NGA/2016/ISTH_0017, Lassa virus/H.sapiens-wt/NGA/2018/ISTH0959, LASV003-NIG-2008, LASV035-NIG-2009, LASV1000-NIG-2009, LASV221-NIG-2010, LASV237-NIG-2010, LASV253-NIG-2011, LASV254-NIG-2011,or Nig08-04, and
(iii) at least one coding RNA encoding at least one antigenic protein that is or is derived from GPC or prefusion-stabilized GPC of a LASV strain from clade III, preferably of strain GA391 or Nig08-A19; and
(iv) at least one coding RNA encoding at least one antigenic protein that is or is derived from GPC or prefusion-stabilized GPC of a LASV strain from clade IV preferably of strain Josiah.

Accordingly, in embodiments, the composition of the second aspect may comprise at least 2, 3, 4, 5, 6, 7, 8, 9, 10 or even more different RNAs of the first aspect, wherein each of said RNAs encodes a different antigenic peptide or protein derived from the same LASV.

In other embodiments, the composition of the second aspect may comprise at least 2, 3, 4, 5, 6, 7, 8, 9, 10 or even more different RNAs of the first aspect, wherein each of said RNAs encodes a different antigenic peptide or protein derived from different proteins of the same LASV.

In other embodiments, the composition of the second aspect may comprise at least 2, 3, 4, 5, 6, 7, 8, 9, 10 or even more different RNAs of the first aspect, wherein each of said RNAs encodes a different antigenic peptide or protein derived from different proteins of different LASV.

In embodiments of the second aspect, the composition comprises at least one RNA encoding at least one antigenic peptide or protein derived from GPC or a variant or fragment thereof and at least one additional RNA encoding at least one antigenic peptide or protein derived from NP (or SP-NP) or a variant or fragment thereof, wherein NP (or SP-NP) may suitably promote T-cell responses (when administered to a subject), wherein GPC and NP (or SP-NP) are derived from the same LASV or from different LASV or combinations thereof.

Preferably, the different LASV belong to different LASV clades or different LASV lineages, preferably to the LASV clades I, II, III, IV, V, and VI or to the LASV lineages I, II, III, IV, V, and VI.

In further embodiments of the second aspect, the composition comprises at least one RNA encoding at least one antigenic peptide or protein derived from GPC or a variant or fragment thereof and at least one additional RNA encoding at least one antigenic peptide or protein derived from SP-NP, preferably from SP-HsPLAT_NP, SP-HsALB_NP, or SP-IgE_NP, or a variant or fragment thereof, wherein SP-NP may suitably promote T-cell responses (when administered to a subject), wherein GPC and SP-NP are derived from the same LASV or from different LASV or combinations thereof. Preferably, the different LASV belong to different LASV clades or different LASV lineages, preferably to the LASV clades or lineages I, II, III, and IV or to the LASV clades or lineages I, II, III, IV, V, and VI.

In particularly preferred embodiments of the second aspect, the composition of the second aspect comprises
at least one artificial RNA which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 2287-3566, 14056-15207 (encoding GPC as defined in the first aspect) and a further artificial RNA which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 9806-11165, 16744-17967, 18104-19327, 19464-20687, 20825-22048 (encoding NP or SP-NP); or
at least one artificial RNA which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 6107-7546, 15208-16743, 23109-24644,24805-26340,26501-28036, 28197-29732, 29893-31428, 31589-33124, 33285-34820, 34981-36516, 36677-38212, 38373-39908, 40069-41604 (encoding prefusion stabilized GPC as defined in the first aspect) and a further artificial RNA which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 9806-11165, 16744-17967, 18104- 19327, 19464-20687, 20825-22048 (encoding NP or SP-NP); or
at least one artificial RNA which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 41765-43300 (encoding GPCmut13) as defined in the first aspect) and a further artificial RNA which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 9806- 11165, 16744-17967, 18104-19327, 19464-20687, 20825-22048 (encoding NP or SP-NP).

In various embodiments, different combinations of GPC or prefusion-stabilized GPC (GPC, GPCmut1, GPCmut1, GPCmut1, GPCmut1, GPCmut1, GPCmut1, GPCmut1, GPCmut1, GPCmut1, GPCmut1, GPCmut1, GPCmut1) RNA constructs and NP (NP, SP-NP) are suitably comprised in the composition.

In preferred embodiments, the composition of the second aspect comprises
(i) at least one coding RNA encoding at least one antigenic protein that is or is derived from GPC or prefusion-stabilized GPC and NP (or SP-NP) of a LASV strain from clade I as specified herein; and
(ii) at least one coding RNA encoding at least one antigenic protein that is or is derived from GPC or prefusion-stabilized GPC and NP (or SP-NP) of a LASV strain from clade II as specified herein; and
(iii) at least one coding RNA encoding at least one antigenic protein that is or is derived from GPC or prefusion-stabilized GPC and NP (or SP-NP) of a LASV strain from clade III as specified herein; and
(iv) at least one coding RNA encoding at least one antigenic protein that is or is derived from GPC or prefusion-stabilized GPC and NP (or SP-NP) of a LASV strain from clade IV as specified herein.

LASV clade members may be derived from Lists 1-6. Moreover, each suitable amino acid sequence or nucleic acid sequence provided herein is provided with information regarding virus strain, clade etc. that can be found under <223> identifier in the sequence listing of the invention. Particularly suitable clade members are provided in Table A below.

TABLE A
Suitable LSAV clade members

| LASV Strain/Isolate | Clade |
|---|---|
| LP | I |
| Pinneo-NIG-1969 | I |
| 803213 | II |
| ISTH0009-NIG-2011 | II |
| ISTH2010-NIG-2012 | II |
| Lassa virus/H. sapiens-wt/NGA/2015/ISTH_0543 | II |
| Lassa virus/H. sapiens-wt/NGA/2016/ISTH_0009 | II |
| Lassa virus/H. sapiens-wt/NGA/2016/ISTH_0017 | II |
| Lassa virus/H. sapiens-wt/NGA/2018/ISTH_0959 | II |
| LASV003-NIG-2008 | II |
| LASV035-NIG-2009 | II |
| LASV1000-NIG-2009 | II |
| LASV221-NIG-2010 | II |
| LASV237-NIG-2010 | II |
| LASV253-NIG-2011 | II |
| LASV254-NIG-2011 | II |
| Nig08-04 | II |
| GA391 | III |
| Nig08-A19 | III |
| Josiah | IV |
| G1180-SLE-2010 | IV |
| G1442-SLE-2011 | IV |
| G3010-SLE-2013 | IV |
| G502-SLE-2009 | IV |
| G733-SLE-2010 | IV |
| MOPV/LASV reassortant IGS-1 | IV |
| LM765-SLE-2012 | IV |
| LM776-SLE-2012 | IV |
| Mad39 | IV |
| Z0947-SLE-2011 | IV |
| AV | V |
| Togo/2016/7082 | VI |
| KAK-428 | n.d. (VII) |

In embodiments of the second aspect, the composition comprises at least one RNA encoding at least one antigenic peptide or protein derived from prefusion-stabilized GPC or a variant or fragment thereof and at least one additional RNA encoding at least one antigenic peptide or protein derived from NP (or SP-NP) or a variant or fragment thereof, wherein NP (or SP-NP) may suitably promote T-cell responses (when administered to a subject), wherein prefusion-stabilized GPC and NP (or SP-NP) are derived from the same LASV or from different LASV or combinations thereof. Preferably, the different LASV belong to different LASV clades or different LASV lineages, preferably to the LASV clades I, II, III, IV, V, and VI or to the LASV lineages I, II, III, IV, V, and VI.

In embodiments of the second aspect, the composition comprises at least one RNA encoding at least one antigenic peptide or protein derived from GPC or a variant or fragment thereof and at least one additional RNA encoding at least one antigenic peptide or protein derived from NP (or SP-NP) or a variant or fragment thereof, and at least one additional RNA encoding at least one antigenic peptide or protein derived from Z or a variant or fragment thereof, wherein GPC, NP (or SP-NP) and Z may suitably promote the formation of VLPs (when administered to a subject), wherein GPC, NP (or SP-NP) and Z are derived from the same LASV or from different LASV or combinations thereof. Preferably, the different LASV belong to different LASV clades or different LASV lineages, preferably to the LASV clades I, II, III, IV, V, and VI or to the LASV lineages I, II, III, IV, V, and VI.

In embodiments of the second aspect, the composition comprises at least one RNA encoding at least one antigenic peptide or protein derived from prefusion-stabilized GPC or a variant or fragment thereof and at least one additional RNA encoding at least one antigenic peptide or protein derived from NP (or SP-NP) or a variant or fragment thereof, and at least one additional RNA encoding at least one antigenic peptide or protein derived from Z or a variant or fragment thereof, wherein GPC, NP (or SP-NP) and Z may suitably promote the formation of VLPs (when administered to a subject), wherein prefusion-stabilized GPC, NP (or SP-NP) and Z are derived from the same LASV or from different LASV or combinations thereof. Preferably, the different LASV belong to different LASV clades or different LASV lineages, preferably to the LASV clades I, II, III, IV, V, and VI or to the LASV lineages I, II, III, IV, V, and VI.

As outlined above, the antigenic peptide(s) or protein(s) may be derived from different LASV clades, in particular from clade I, II, III, IV, V, and VI and/or from a LASV of lineage I, II, III, IV, V, and VI. Such embodiments may have the advantage that the composition, when administered to the subject, provides broad protection against different LASV clades which is important in the context of an effective LASV vaccine. Different suitable LASV clade members are provided in Lists 1-6, and in particular in Table A, showing particularly preferred LASV strains. Further particularly preferred strains are LP (clade I), 803213 (clade II), GA391 (clade III) and Josiah (clade IV).

For the production of a composition comprising at least 2, 3, 4, 5, 6, 7, 8, 9, 10 RNA constructs of the first aspect, methods as disclosed in published patent application WO2017/1090134 are preferably used and adapted accordingly.

It has to be understood that in the context of the invention, certain combinations of coding sequences may be generated by any combination of monocistronic, bicistronic and multicistronic nucleic acids and/or multi- antigen-constructs/nucleic acid to obtain a nucleic acid composition encoding multiple antigenic peptides or proteins as defined herein.

In embodiments, one or more compatible solid or liquid fillers or diluents or encapsulating compounds may be used as well, which are suitable for administration to a subject. The term “compatible” as used herein means that the constituents of the composition are capable of being mixed with the at least one RNA and, optionally, a plurality of RNAs of the composition, in such a manner that no interaction occurs, which would substantially reduce the biological activity or the pharmaceutical effectiveness of the composition under typical use conditions. Pharmaceutically acceptable carriers, fillers and diluents must have sufficiently high purity and sufficiently low toxicity to make them suitable for administration to a subject to be treated. Compounds which may be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

Complexation:

In a preferred embodiment of the second aspect, at least one RNA is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof. In embodiments where more than one or a plurality, e.g. 2, 3, 4, 5, 6, 7, 8, 9, 10, 11, 12, 13, 14, 15 of the RNAs are comprised in the composition, said more than one or a plurality, e.g. 2, 3, 4, 5, 6, 7, 8, 9, 10, 11, 12, 13, 14, 15 of the RNAs may be complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof as described in the following.

Accordingly, embodiments relating to “at least one RNA” may likewise be read on and be understood as suitable embodiments of more than one or a plurality, e.g. 2, 3, 4, 5, 6, 7, 8, 9, 10, 11, 12, 13, 14, 15 of the RNAs as specified herein.

The term “cationic or polycationic compound” as used herein will be recognized and understood by the person of ordinary skill in the art, and are e.g. intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, p151, FGF, Lactoferrin, Transportan, Buforin-2, Bac715-24, SynB, SynB(1), pVEC, hCT-derived peptides, SAP, or histones. More preferably, the nucleic acid as defined herein, preferably the mRNA as defined herein, is complexed with one or more polycations, preferably with protamine or oligofectamine, most preferably with protamine.

In a preferred embodiment of the second aspect, the at least one RNA is complexed with protamine.

Further preferred cationic or polycationic compounds, which can be used as complexation agent may include cationic polysaccharides, e.g. chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In this context it is particularly preferred that the at least one RNA is complexed or at least partially complexed with a cationic or polycationic compound and/or a polymeric carrier, preferably cationic proteins or peptides. In this context, the disclosure of WO2010/037539 and WO2012/113513 is incorporated herewith by reference. Partially means that only a part of the nucleic acid is complexed with a cationic compound and that the rest of the nucleic acid is (comprised in the inventive (pharmaceutical) composition) in uncomplexed form (“free”).

In a preferred embodiment of the second aspect, the at least one RNA is complexed with one or more cationic or polycationic compounds, preferably protamine, and at least one free RNA.

In this context it is particularly preferred that the at least one RNA is complexed, or at least partially complexed with protamine. Preferably, the molar ratio of the nucleic acid, particularly the RNA of the protamine-complexed RNA to the free RNA may be selected from a molar ratio of about 0.001:1 to about 1:0.001, including a ratio of about 1:1. Suitably, the complexed RNA is complexed with protamine by addition of protamine-trehalose solution to the RNA sample at a RNA:protamine weight to weight ratio (w/w) of 2:1.

Further preferred cationic or polycationic proteins or peptides that may be used for complexation can be derived from formula (Arg)I;(Lys)m;(His)n;(Orn)o;(Xaa)x of the patent application WO2009/030481 or WO2011/026641, the disclosure of WO2009/030481 or WO2011/026641 relating thereto incorporated herewith by reference.

In a preferred embodiment of the second aspect, the at least one RNA is complexed or at least partially complexed with at least one cationic or polycationic proteins or peptides preferably selected from SEQ ID NOs: 13868-13872, or any combinations thereof.

According to embodiments, the composition of the present invention comprises at least one RNA as defined herein, and a polymeric carrier.

The term “polymeric carrier” as used herein will be recognized and understood by the person of ordinary skill in the art, and are e.g. intended to refer to a compound that facilitates transport and/or complexation of another compound (cargo). A polymeric carrier is typically a carrier that is formed of a polymer. A polymeric carrier may be associated to its cargo (e.g. RNA) by covalent or non-covalent interaction.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are crosslinked by disulfide bonds (via -SH groups).

In this context, polymeric carriers according to formula {(Arg)I;(Lys)m;(His)n;(Orn)o;(Xaa′)x(Cys)y} and formula Cys,{(Arg)I;(Lys)m;(His)n;(Orn)o;(Xaa)x}Cys₂ of WO2012/013326 are preferred, the disclosure of WO2012/013326 relating thereto incorporated herewith by reference.

In embodiments, the polymeric carrier used to complex the at least one RNA as defined herein may be derived from a polymeric carrier molecule according formula (L-P¹-S—[S—P²—S]ₙ—S—P³-L) of WO2011/026641, the disclosure of WO2011/026641 relating thereto incorporated herewith by reference.

In embodiments, the polymeric carrier compound is formed by, or comprises or consists of the peptide elements CysArg12Cys (SEQ ID NO: 13868) or CysArg12 (SEQ ID NO: 13869) or TrpArg12Cys (SEQ ID NO: 13870). In particularly preferred embodiments, the polymeric carrier compound consists of a (R₁₂C)-(R₁₂C) dimer, a (WR₁₂C)-(WR₁₂C) dimer, or a (CR12)-(CR12C)-(CR12) trimer, wherein the individual peptide elements in the dimer (e.g. (WR12C)), or the trimer (e.g. (CR12)), are connected via —SH groups.

In a preferred embodiment of the second aspect, the at least one RNA is complexed or associated with a polyethylene glycol/peptide polymer comprising HO-PEG5000-S—(S—CHHHHHHRRRRHHHHHHC—S-)7-S-PEG5000-OH (SEQ ID NO: 13871 as peptide monomer).

In a further preferred embodiment of the second aspect, the at least one artificial RNA of the first aspect and, optionally, the further artificial RNA of the second aspect, is complexed or associated with a polyethylene glycol/peptide polymer comprising HO-PEG5000-S—(S—CHHHHHHRRRRHHHHHHC—S-)4-S-PEG5000-OH (SEQ ID NO: 13871 as peptide monomer).

In a further preferred embodiment of the second aspect, the at least one artificial RNA of the first aspect and, optionally, the further artificial RNA of the second aspect, is complexed or associated with a polyethylene glycol/peptide polymer comprising HO-PEG5000-S—(S—CGHHHHHRRRRHHHHHGC—S-)7-S-PEG5000-OH (SEQ ID NO: 13872 as peptide monomer).

In a further preferred embodiment of the second aspect, the at least one artificial RNA of the first aspect and, optionally, the further artificial RNA of the second aspect, is complexed or associated with a polyethylene glycol/peptide polymer comprising HO-PEG5000-S—(S—CGHHHHHRRRRHHHHHGC-S-)4-S-PEG5000-OH (SEQ ID NO: 13872 as peptide monomer).

In other embodiments, the composition comprises at least one RNA, wherein the at least one RNA is complexed or associated with polymeric carriers and, optionally, with at least one lipid component as described in WO2017/212008A1, WO2017/212006A1, WO2017/212007A1, and WO2017/212009A1. In this context, the disclosures of WO2017/212008A1, WO2017/212006A1, WO2017/212007A1, and WO2017/212009A1 are herewith incorporated by reference.

In a particularly preferred embodiment, the polymeric carrier is a peptide polymer, preferably a polyethylene glycol/peptide polymer as defined above, and a lipid component, preferably a lipidoid component, more preferably lipidoid component.

A lipidoid compound, also simply referred to as lipidoid, is a lipid-like compound, i.e. an amphiphilic compound with lipid-like physical properties. In the context of the present invention the term lipid is considered to also encompass lipidoid compounds.

In preferred embodiment of the second aspect, the at least one RNA is complexed or associated with a polymeric carrier, preferably with a polyethylene glycol/peptide polymer as defined above, and a lipidoid component, wherein the lipidoid component is a compound according to formula A

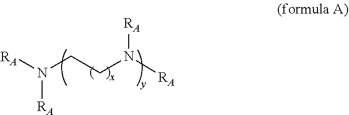

wherein
R_{A} is independently selected for each occurrence an unsubstituted, cyclic or acyclic, branched or unbranched C₁₋₂₀ aliphatic group; a substituted or unsubstituted, cyclic or acyclic, branched or unbranched C₁₋₂₀ heteroaliphatic group; a substituted or unsubstituted aryl; a substituted or unsubstituted heteroaryl;

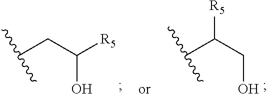

wherein at least one R_{A} is

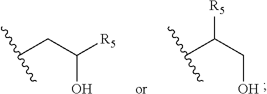

R₅ is independently selected for each occurrence of from an unsubstituted, cyclic or acyclic, branched or unbranched C₈₋₁₆ aliphatic; a substituted or unsubstituted aryl; or a substituted or unsubstituted heteroaryl;
each occurrence of x is an integer from 1 to 10;
each occurrence of y is an integer from 1 to 10;
or a pharmaceutically acceptable salt thereof.

In a preferred embodiment, the lipidoid component is 3-C12-OH according to formula B

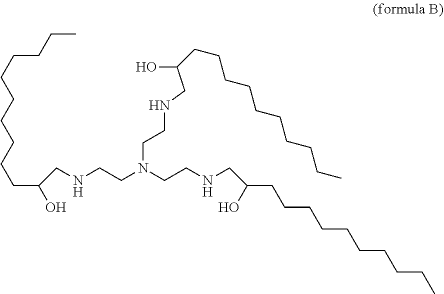 [SMILES: CCCCCCCCCCC(O)CNCCN(CCNCC(O)CCCCCCCCCC)CCNCC(O)CCCCCCCCCC]

In preferred embodiments, the peptide polymer comprising lipidoid 3-C12-OH as specified above is used to complex the at least one RNA of the first aspect to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of WO2017/212009A1, in particular claims 1 to 10 of W02017/212009A1, and the specific disclosure relating thereto is herewith incorporated by reference.

Encapsulation/Complexation in LNPs:

In preferred embodiments of the second aspect, the at least one RNA is complexed or associated with one or more lipids (e.g. cationic lipids and/or neutral lipids), thereby forming liposomes, lipid nanoparticles (LNPs), lipoplexes, and/or nanoliposomes.

For compositions comprising more than one artificial RNA construct as defined herein (e,g, GPC and NP; or e.g. at least 2 GPCs or 2 prefusion-stbilized GPCs of different strains), said constructs may be co-formulated in e.g. LNPs to form the respective composition.

Alternatively, said more than one RNA constructs may be formulated separately, and may subsequently be combined, to form the respective composition.

In this context, the terms “complexed” or “associated” refer to the essentially stable combination of RNA of the first aspect with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of RNA. E.g., a liposome, a lipid complex, a lipoplex and the like are within the scope of an LNP.

Accordingly, in preferred embodiments of the second aspect, the at least one RNA is complexed with one or more lipids thereby forming lipid nanoparticles (LNP).

LNPs typically comprise a cationic lipid and one or more excipient selected from neutral lipids, charged lipids, steroids and polymer conjugated lipids (e.g. PEGylated lipid). The at least one RNA, or the plurality of RNAs may be encapsulated in the lipid portion of the LNP or an aqueous space enveloped by some or the entire lipid portion of the LNP. The at least one RNA or a portion thereof may also be associated and complexed with the LNP. An LNP may comprise any lipid capable of forming a particle to which the nucleic acids are attached, or in which the one or more nucleic acids are encapsulated. Preferably, the LNP comprising nucleic acids comprises one or more cationic lipids, and one or more stabilizing lipids. Stabilizing lipids include neutral lipids and PEGylated lipids.

The cationic lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

The LNP may comprise any further cationic or cationisable lipid, i.e. any of a number of lipid species which carry a net positive charge at a selective pH, such as physiological pH.

Such lipids include, but are not limited to, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, HGT4003, 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing.

In some embodiments, the lipid is selected from the group consisting of 98N12-5, C12-200, and ckk-E12.

In one embodiment, the further cationic lipid is an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In one embodiment, the at least one RNA may be formulated in an aminoalcohol lipidoid. Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531A1, hereby incorporated by reference.

In another embodiment, ionizable lipids can also be the compounds as disclosed in WO2015/074085A1 (i.e. ATX-001 to ATX-032 or the compounds as specified in claims 1-26), U.S. Appl. No. 61/905,724 and Ser. No. 15/614,499 or U.S. Pat. Nos. 9,593,077 and 9,567,296 hereby incorporated by reference in their entirety.

In that context, any lipid derived from generic formula (X1)

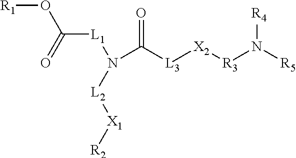

wherein, Ri and R2 are the same or different, each a linear or branched alkyl consisting of 1 to 9 carbons, an alkenyl or alkynyl consisting of 2 to 11carbons, Li and L2 are the same or different, each a linear alkylene or alkenylene consisting of 5 to 18 carbons, or forming a heterocycle with N, Xi is a bond, or is —CO—O— whereby -L2-CO—O—R2 is formed, X2 is S or O, L3 is a bond or a linear or branched alkylene consisting of 1 to 6 carbons, or forming a heterocycle with N, R3 is a linear or branched alkylene consisting of 1 to 6 carbons, and R4 and R 5 are the same or different, each hydrogen or a linear or branched alkyl consisting of 1 to 6 carbons; or a pharmaceutically acceptable salt thereof may be suitably used.

In other embodiments, suitable cationic lipids can also be the compounds as disclosed in WO2017/117530A1 (i.e. lipids 13, 14, 15, 16, 17, 18, 19, 20, or the compounds as specified in the claims), hereby incorporated by reference in its entirety.

In that context, any lipid derived from generic formula (X2)

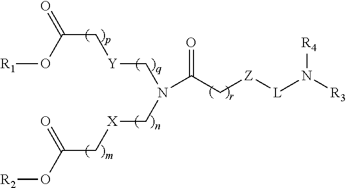

wherein

X is a linear or branched alkylene or alkenylene, monocyclic, bicyclic, or tricyclic arene or heteroarene;

Y is a bond, an ethene, or an unsubstituted or substituted aromatic or heteroaromatic ring; Z is S or 0;

L is a linear or branched alkylene of 1 to 6 carbons;

R-3 and R4 are independently a linear or branched alkyl of 1 to 6 carbons;

Ri and R2 are independently a linear or branched alkyl or alkenyl of 1 to 20 carbons; r is 0 to 6; and

m, n, p, and q are independently 1 to 18;

wherein when n=q, m=p, and Ri=R2, then X and Y differ;

wherein when X=Y, n=q, m=p, then Ri and R2 differ;

wherein when X=Y, n=q, and Ri=R2, then m and p differ; and

wherein when X=Y, m=p, and Ri=R2, then n and q differ;

or a pharmaceutically acceptable salt thereof.

In preferred embodiments, a lipid may be used derived from formula (X2), wherein, X is a bond, linear or branched alkylene, alkenylene, or monocyclic, bicyclic, or tricyclic arene or heteroarene; Y is a monocyclic, bicyclic, or tricyclic arene or heteroarene; Z is S or O; L is a linear or branched alkylene of 1 to 6 carbons; R3 and R4 are independently a linear or branched alkyl of 1 to 6 carbons; Ri and R2 are independently a linear or branched alkyl or alkenyl of 1 to 20 carbons; r is 0 to 6; and m, n, p, and q are independently 1 to 18; or a pharmaceutically acceptable salt thereof may be suitably used.

In preferred embodiments, ionizable lipids may also be selected from the lipids disclosed in WO2018078053A1 (i.e. lipids derived form formula I, II, and III of WO2018078053A1, or lipids as specified in claims 1 to 12 of WO2018078053A1), the disclosure of WO2018078053A1 hereby incorporated by reference in its entirety. In that context, lipids disclosed in Table 7 of WO2018078053A1 (e.g. lipids derived from formula I-1 to 1-41) and lipids disclosed in Table 8 of WO2018078053A1 (e.g. lipids derived from formula 11-1 to 11-36) may be suitably used in the context of the invention. Accordingly, formula I-1 to formula 1-41 and formula 11-1 to formula 11-36 of WO2018078053A1, and the specific disclosure relating thereto, are herewith incorporated by reference.

In particularly preferred embodiments of the second aspect, a suitable lipid may be a cationic lipid according to formula (III)

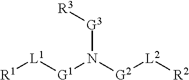

or a pharmaceutically acceptable salt, tautomer, prodrug or stereoisomer thereof, wherein, R1 , R2 , R3, L1, L2, G1, G2, and G3 are as below.

Formula (III) is further defined in that: one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}—or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O—or a direct bond;
G¹ and G² are each independently unsubstituted C₁-C₁₂ alkylene or C₁-C₁₂ alkenylene;
G³ is C₁-C₂₄ alkylene, alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of formula (III), the lipid has one of the following structures (IIIA) or (IIIB):

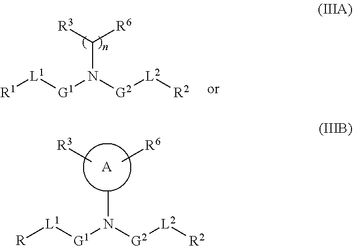

wherein:

A is a 3 to 8-membered cycloalkyl or cycloalkylene ring; R⁶ is, at each occurrence, independently H, OH or C₁-C₂₄ alkyl; n is an integer ranging from 1 to 15.

In some of the foregoing embodiments of formula (III), the lipid has structure (IIIA), and in other embodiments, the lipid has structure (IIIB).

In other embodiments of formula (III), the lipid has one of the following structures (IIIC) or (IIID):

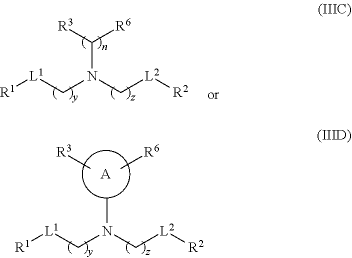

wherein y and z are each independently integers ranging from 1 to 12.

In any of the foregoing embodiments of formula (III), one of L¹ or L² is —O(C═O)—. E.g., in some embodiments each of L¹ and L² are —O(C═O)—. In some different embodiments of any of the foregoing, Land L² are each independently —(C═O)O— or —O(C═O)—. E.g., in some embodiments each of L¹ and L² is —(C═O)O—.

In preferred embodiments of the second aspect, the cationic lipid of the LNP is a compound of formula III, wherein:

L¹ and L² are each independently —O(C═O)— or (C═O)—O—;

G³ is C₁-C₂₄ alkylene or C₁-C₂₄ alkenylene; and

R³ is H or OR⁵.

In some different embodiments of formula (III), the lipid has one of the following structures (IIIE) or (IIIF):

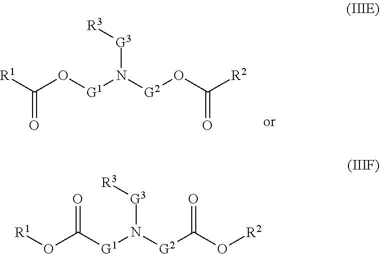

In some of the foregoing embodiments of formula (III), the lipid has one of the following structures (IIIG), (IIIH), (IIII), or (IIIJ):

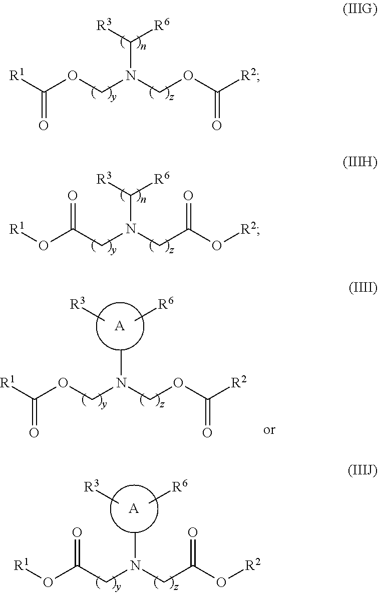

In some of the foregoing embodiments of formula (III), n is an integer ranging from 2 to 12, e.g. from 2 to 8 or from 2 to 4. In some embodiments, n is 3, 4, 5 or 6. In some embodiments, n is 3. In some embodiments, n is 4. In some embodiments, n is 5. In some embodiments, n is 6. In some other of the foregoing embodiments of formula (III), y and z are each independently an integer ranging from 2 to 10. E.g., in some embodiments, y and z are each independently an integer ranging from 4 to 9 or from 4 to 6. In some of the foregoing embodiments of formula (III), R⁶ is H. In other of the foregoing embodiments, R⁶ is C₁-C₂₄ alkyl. In other embodiments, R⁶ is OH. In some embodiments of formula (III), G³ is unsubstituted. In other embodiments, G3 is substituted. In various different embodiments, G³ is linear C₁-C₂₄ alkylene or linear C₁-C₂₄ alkenylene. In some other foregoing embodiments of formula (III), R¹ or R², or both, is C₆-C₂₄ alkenyl. E.g., in some embodiments, R¹ and R² each, independently have the following structure:

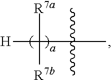

wherein:

R⁷¹ and R^{7b} are, at each occurrence, independently H or C₁-C₁₂ alkyl; and a is an integer from 2 to 12, wherein

R^{7a}, R^{7b} and a are each selected such that R¹ and R² each independently comprise from 6 to 20 carbon atoms. E.g., in some embodiments a is an integer ranging from 5 to 9 or from 8 to 12. In some of the foregoing embodiments of formula (III), at least one occurrence of R^{7a} is H. In some embodiments, R^{7a} is H at each occurrence. In other different embodiments of the foregoing, at least one occurrence of R^{7b} is C₁-C₈ alkyl. E.g., in some embodiments, C₁-C₈ alkyl is methyl, ethyl, n-propyl, iso-propyl, n-butyl, iso-butyl, tert-butyl, n-hexyl or n-octyl.

In different embodiments of formula (III), R¹ or R², or both, has one of the following structures:

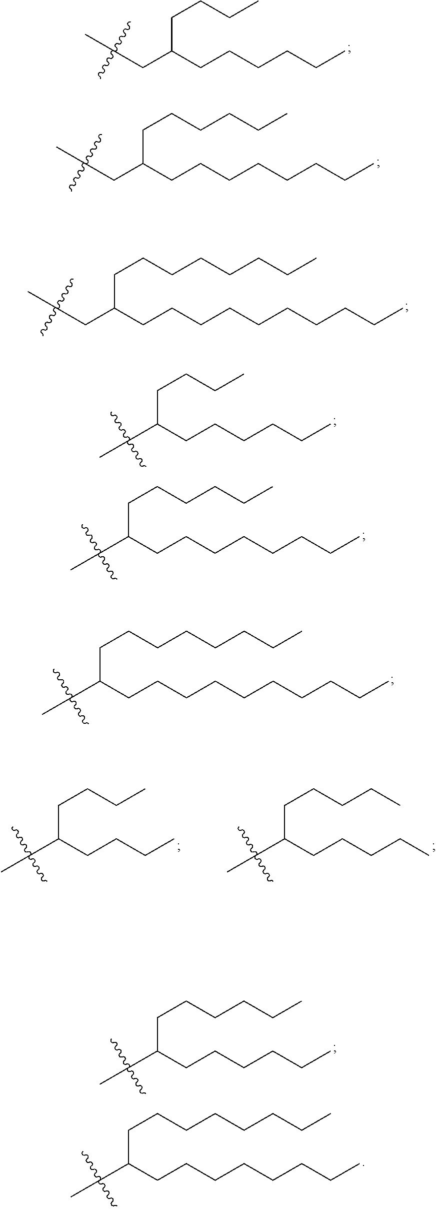

In preferred embodiments of the second aspect, the cationic lipid of the LNP is a compound of formula III, wherein:
L¹ and L² are each independently —O(C═O)— or (C═O)—O—; and
R¹ and R² each independently have one of the following structures:

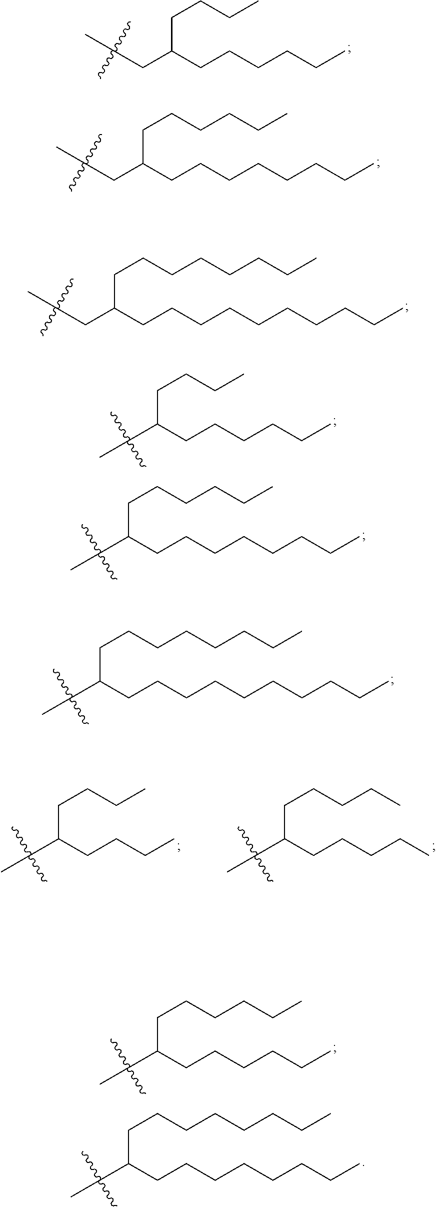

In some of the foregoing embodiments of formula (III), R³ is OH, CN, —C(═O)OR⁴, —OC(═O)R⁴ or —NHC(═O)R⁴.

In some embodiments, R⁴ is methyl or ethyl.

In preferred embodiments of the second aspect, the cationic lipid of the LNP is a compound of formula III, wherein R³ is OH.

In particularly preferred embodiment of the second aspect, the at least one RNA is complexed with one or more lipids thereby forming LNPs, wherein the cationic lipid of the LNP is selected from structures III-1 to III-36 (see Table 6).

TABLE 6
Representative lipid compounds derived from formula (III)

| No. | Structure |
|---|---|
| III-1 | 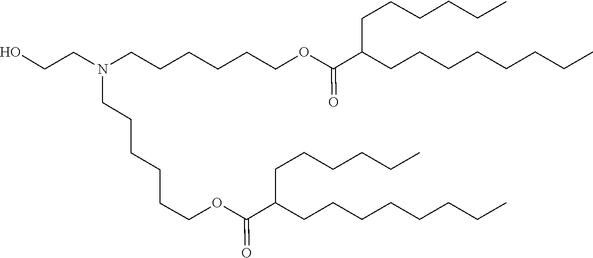 [SMILES: CCCCCCCCC(CCCCCC)C(=O)OCCCCCCN(CCO)CCCCCCOC(=O)C(CCCCCC)CCCCCCCC] |
| III-2 | 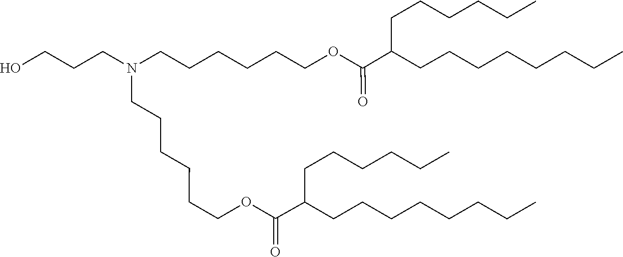 [SMILES: CCCCCCCCC(CCCCCC)C(=O)OCCCCCCN(CCCO)CCCCCCOC(=O)C(CCCCCC)CCCCCCCC] |
| III-3 | 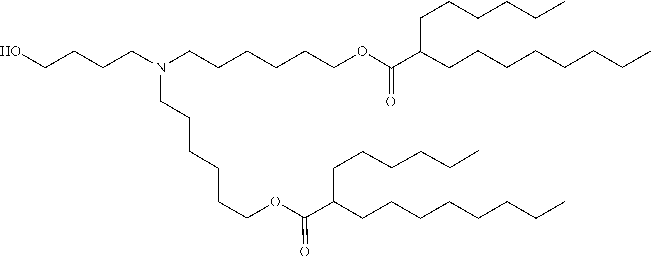 [SMILES: CCCCCCCCC(CCCCCC)C(=O)OCCCCCCN(CCCCO)CCCCCCOC(=O)C(CCCCCC)CCCCCCCC] |
| III-4 | 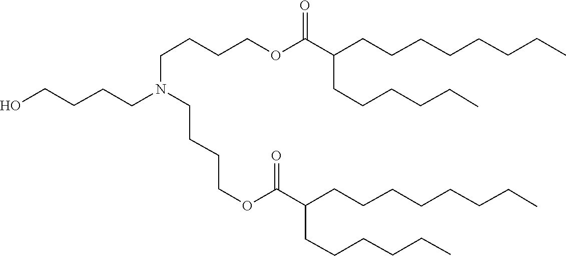 [SMILES: CCCCCCCCC(CCCCCC)C(=O)OCCCCN(CCCCO)CCCCOC(=O)C(CCCCCC)CCCCCCCC] |
| III-5 | 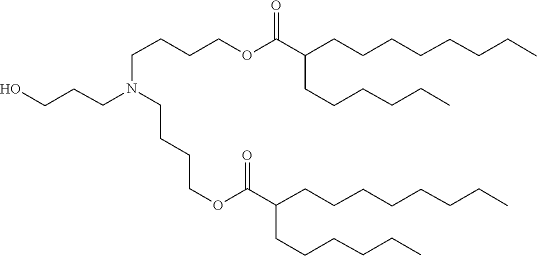 [SMILES: CCCCCCCCC(CCCCCC)C(=O)OCCCCN(CCCO)CCCCOC(=O)C(CCCCCC)CCCCCCCC] |
| III-6 | 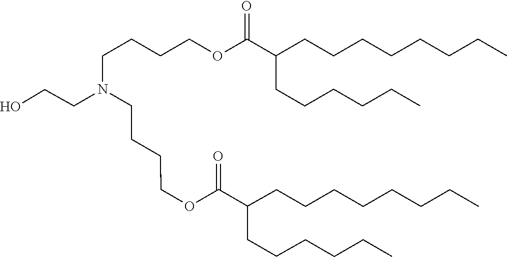 [SMILES: CCCCCCCCC(CCCCCC)C(=O)OCCCCN(CCO)CCCCOC(=O)C(CCCCCC)CCCCCCCC] |
| III-7 | 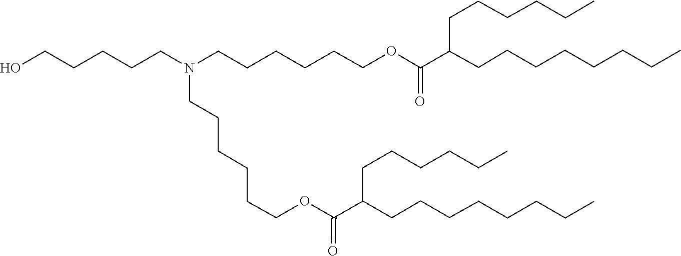 [SMILES: CCCCCCCCC(CCCCCC)C(=O)OCCCCCCN(CCCCCO)CCCCCCOC(=O)C(CCCCCC)CCCCCCCC] |
| III-8 | 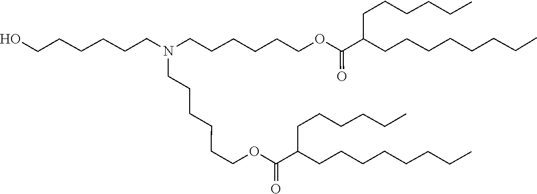 [SMILES: CCCCCCCCC(CCCCCC)C(=O)OCCCCCCN(CCCCCCO)CCCCCCOC(=O)C(CCCCCC)CCCCCCCC] |
| III-9 | 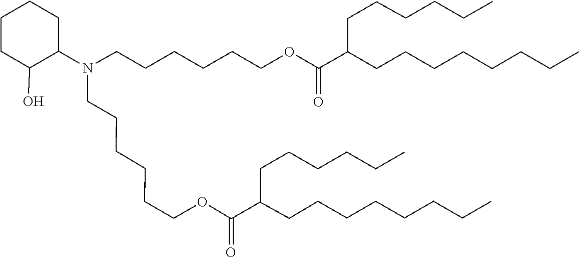 [SMILES: CCCCCCCCC(CCCCCC)C(=O)OCCCCCCN(CCCCCCOC(=O)C(CCCCCC)CCCCCCCC)C1CCCCC1O] |
| III-10 | 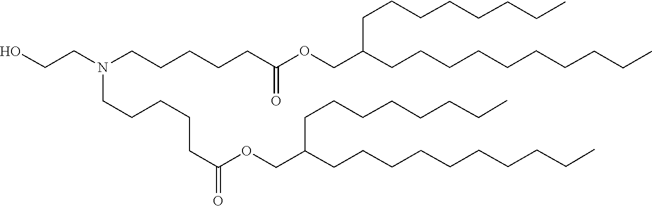 [SMILES: CCCCCCCCCCC(CCCCCCCC)COC(=O)CCCCCN(CCO)CCCCCC(=O)OCC(CCCCCCCC)CCCCCCCCCC] |
| III-11 | 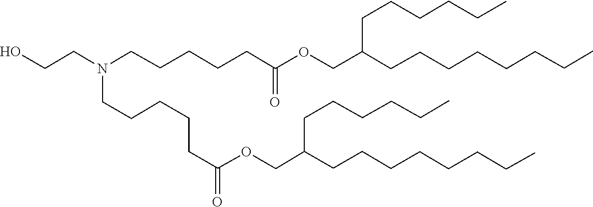 [SMILES: CCCCCCCCC(CCCCCC)COC(=O)CCCCCN(CCO)CCCCCC(=O)OCC(CCCCCC)CCCCCCCC] |
| III-12 | 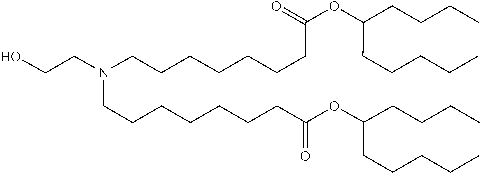 [SMILES: CCCCCC(CCCC)OC(=O)CCCCCCCN(CCO)CCCCCCCC(=O)OC(CCCC)CCCCC] |
| III-13 | 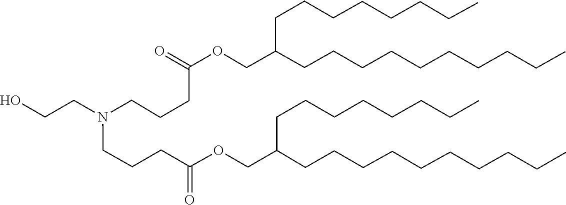 [SMILES: CCCCCCCCCCC(CCCCCCCC)COC(=O)CCCN(CCO)CCCC(=O)OCC(CCCCCCCC)CCCCCCCCCC] |
| III-14 | 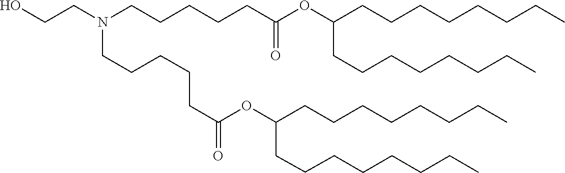 [SMILES: CCCCCCCCC(CCCCCCCC)OC(=O)CCCCCN(CCO)CCCCCC(=O)OC(CCCCCCCC)CCCCCCCC] |
| III-15 | 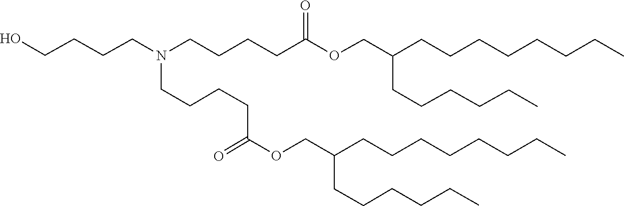 [SMILES: CCCCCCCCC(CCCCCC)COC(=O)CCCCN(CCCCO)CCCCC(=O)OCC(CCCCCC)CCCCCCCC] |
| III-16 | 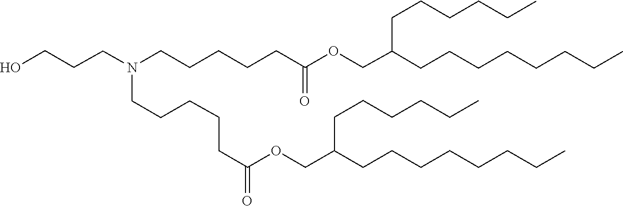 [SMILES: CCCCCCCCC(CCCCCC)COC(=O)CCCCCN(CCCO)CCCCCC(=O)OCC(CCCCCC)CCCCCCCC] |
| III-17 | 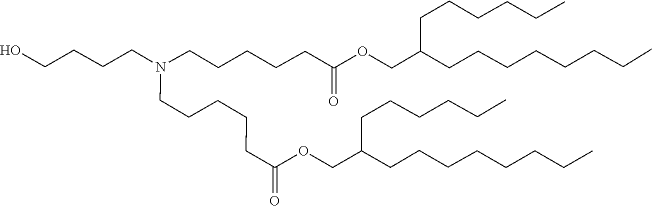 [SMILES: CCCCCCCCC(CCCCCC)COC(=O)CCCCCN(CCCCO)CCCCCC(=O)OCC(CCCCCC)CCCCCCCC] |
| III-18 | 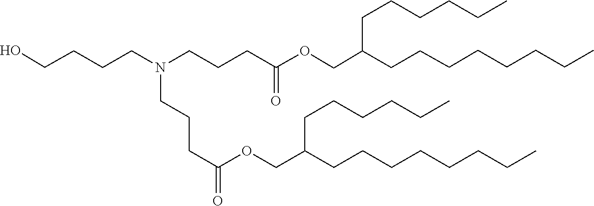 [SMILES: CCCCCCCCC(CCCCCC)COC(=O)CCCN(CCCCO)CCCC(=O)OCC(CCCCCC)CCCCCCCC] |
| III-19 | 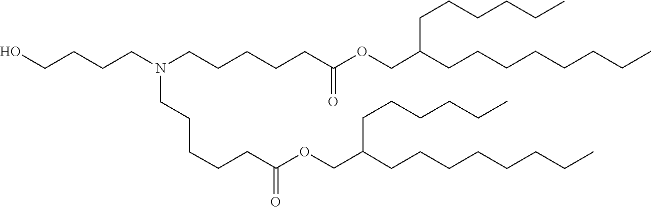 [SMILES: CCCCCCCCC(CCCCCC)COC(=O)CCCCCN(CCCCO)CCCCCC(=O)OCC(CCCCCC)CCCCCCCC] |
| III-20 | 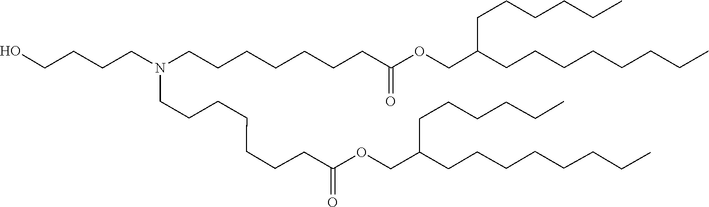 [SMILES: CCCCCCCCC(CCCCCC)COC(=O)CCCCCCCN(CCCCO)CCCCCCCC(=O)OCC(CCCCCC)CCCCCCCC] |
| III-21 | 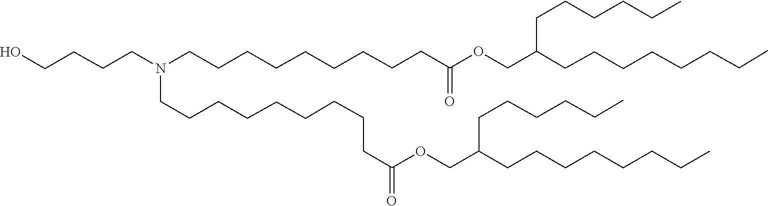 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 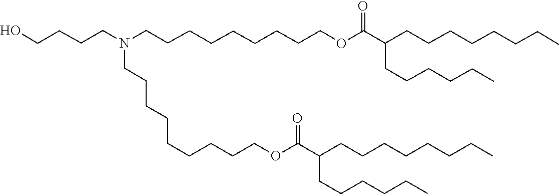 [SMILES: CCCCCCCCC(CCCCCC)C(=O)OCCCCCCCCCN(CCCCO)CCCCCCCCCOC(=O)C(CCCCCC)CCCCCCCC] |
| III-23 | 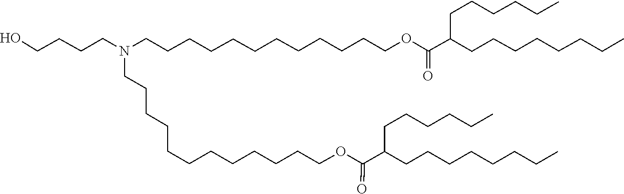 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 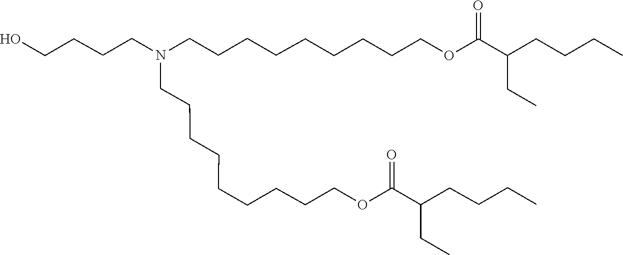 [SMILES: CCCCC(CC)C(=O)OCCCCCCCCCN(CCCCO)CCCCCCCCCOC(=O)C(CC)CCCC] |
| III-25 | 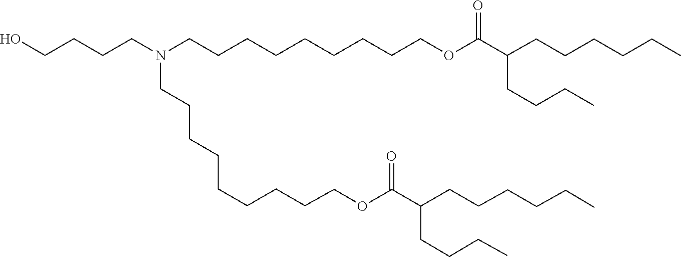 [SMILES: CCCCCCC(CCCC)C(=O)OCCCCCCCCCN(CCCCO)CCCCCCCCCOC(=O)C(CCCC)CCCCCC] |
| III-26 | 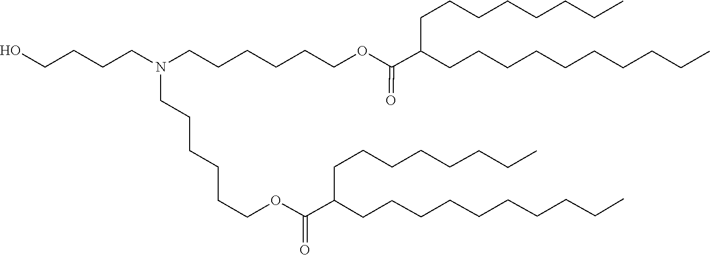 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 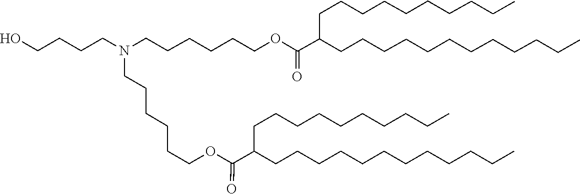 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 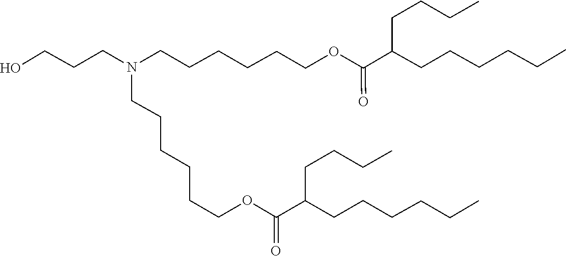 [SMILES: CCCCCCC(CCCC)C(=O)OCCCCCCN(CCCO)CCCCCCOC(=O)C(CCCC)CCCCCC] |
| III-29 | 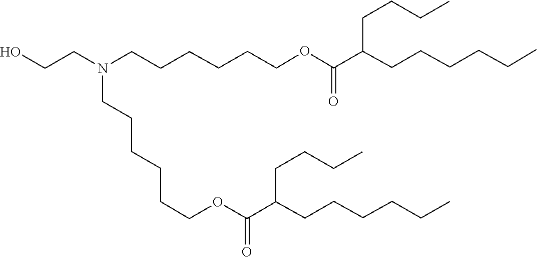 [SMILES: CCCCCCC(CCCC)C(=O)OCCCCCCN(CCO)CCCCCCOC(=O)C(CCCC)CCCCCC] |
| III-30 | 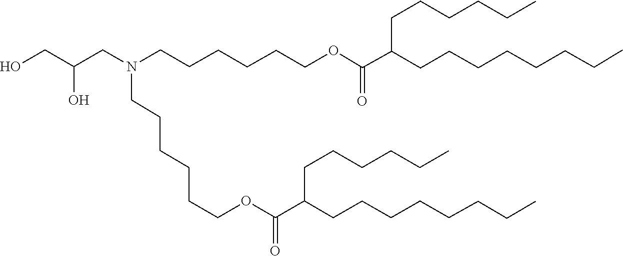 [SMILES: CCCCCCCCC(CCCCCC)C(=O)OCCCCCCN(CCCCCCOC(=O)C(CCCCCC)CCCCCCCC)CC(O)CO] |
| III-31 | 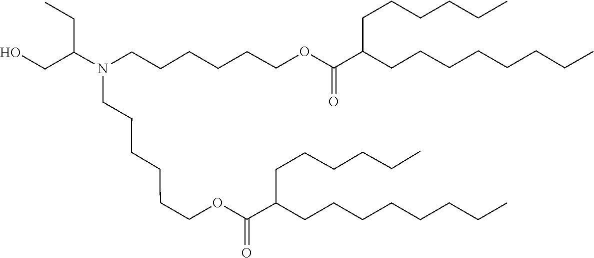 [SMILES: CCCCCCCCC(CCCCCC)C(=O)OCCCCCCN(CCCCCCOC(=O)C(CCCCCC)CCCCCCCC)C(CC)CO] |
| III-32 | 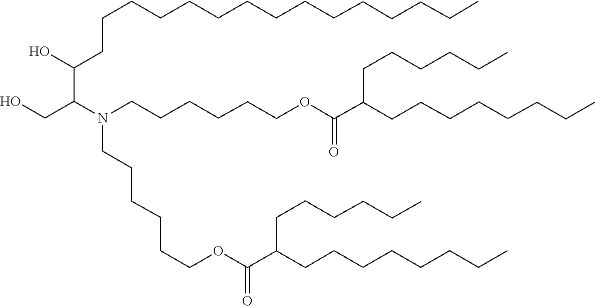 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 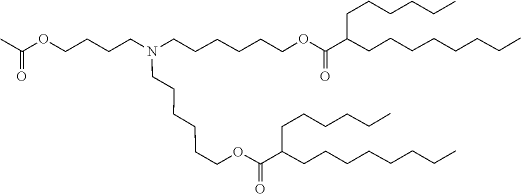 [SMILES: CCCCCCCCC(CCCCCC)C(=O)OCCCCCCN(CCCCCCOC(=O)C(CCCCCC)CCCCCCCC)CCCCOC(C)=O] |
| III-34 | 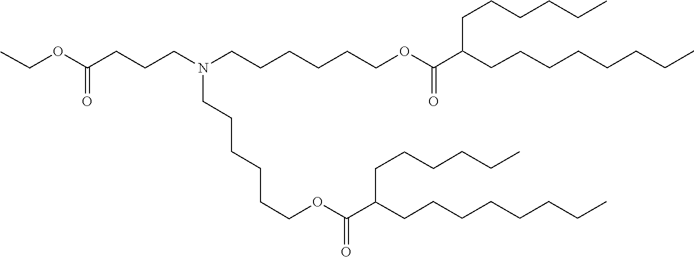 [SMILES: CCCCCCCCC(CCCCCC)C(=O)OCCCCCCN(CCCCCCOC(=O)C(CCCCCC)CCCCCCCC)CCCC(=O)OCC] |
| III-35 | 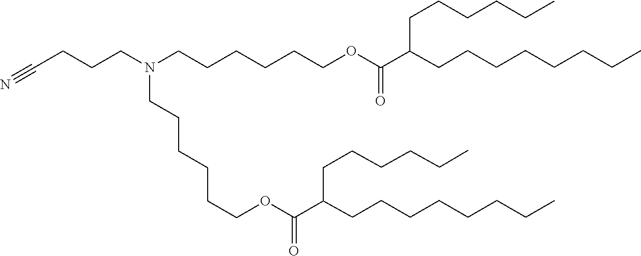 [SMILES: CCCCCCCCC(CCCCCC)C(=O)OCCCCCCN(CCCC#N)CCCCCCOC(=O)C(CCCCCC)CCCCCCCC] |
| III-36 | 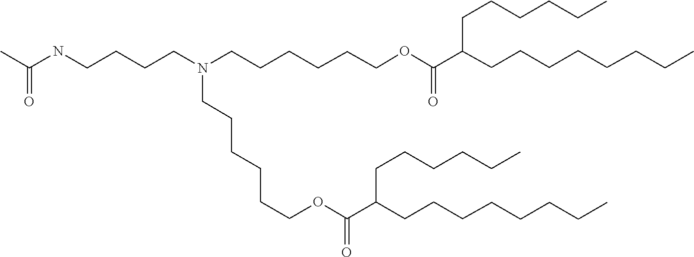 |

In some embodiments, the LNP comprises a lipid of formula (III), at least one RNA of the first aspect, and one or more excipient selected from neutral lipids, steroids and PEGylated lipids. In some embodiments the lipid of formula (III) is compound III-3. In some embodiments the lipid of formula (III) is compound III-7.

In preferred embodiments, the LNP comprises a cationic lipid selected from:

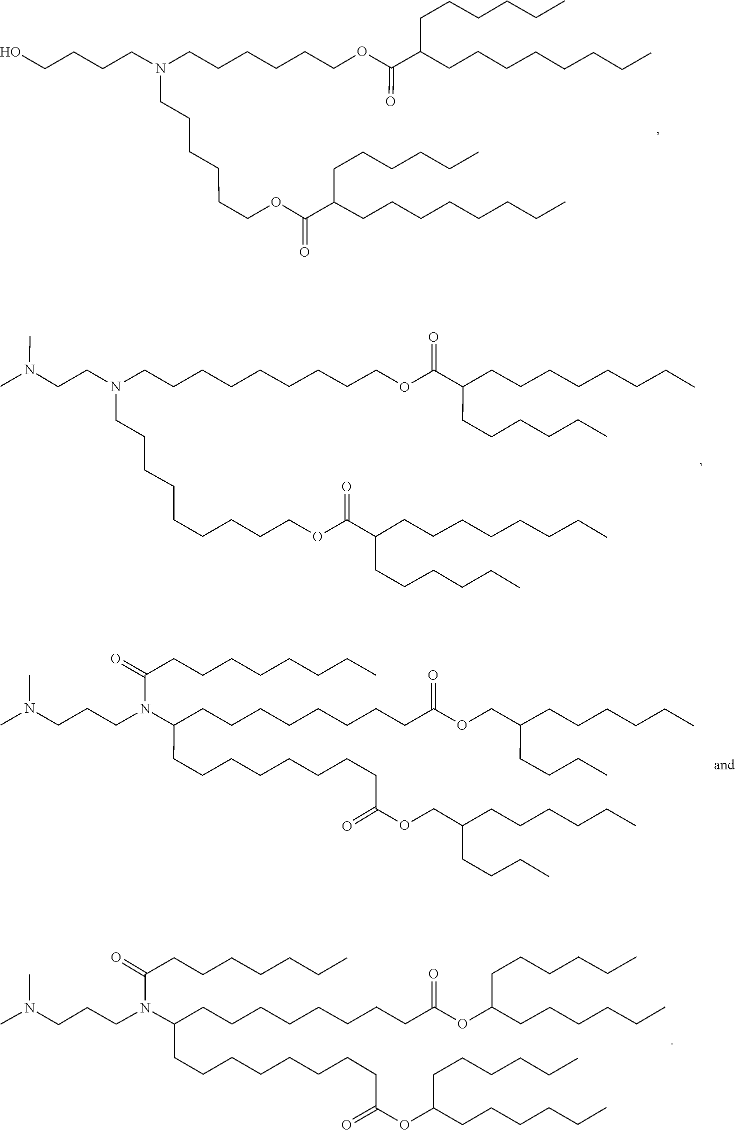

In particularly preferred embodiment of the second aspect, the at least one RNA is complexed with one or more lipids thereby forming LNPs, wherein the LNP comprises the following cationic lipid (lipid according to formula III-3 of Table 6):

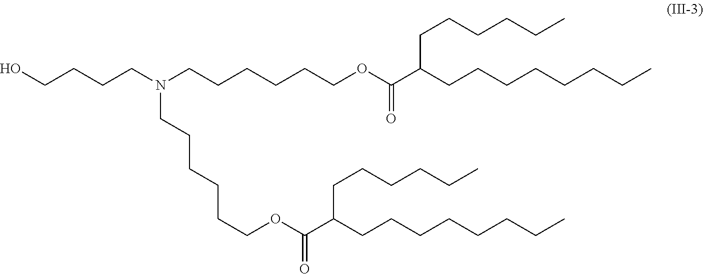 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid as defined herein, preferably as disclosed in Table 6, more preferably cationic lipid compound III-3, is present in the LNP in an amount from about 30 to about 95 mole percent, relative to the total lipid content of the LNP. If more than one cationic lipid is incorporated within the LNP, such percentages apply to the combined cationic lipids.

In one embodiment, the cationic lipid is present in the LNP in an amount from about 30 to about 70 mole percent. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the LNP in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.7 mole percent are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the LNP). In some embodiments, the ratio of cationic lipid to RNA is from about 3 to about 15, such as from about 5 to about 13 or from about 7 to about 11.

In some embodiments of the invention the LNP comprises a combination or mixture of any the lipids described above.

Other suitable (cationic) lipids are disclosed in WO2009/086558, WO2009/127060, WO2010/048536, WO2010/054406, WO2010/088537, WO2010/129709, WO2011/153493, US2011/0256175, US2012/0128760, US2012/0027803, US8158601, WO2016/118724, WO2016/118725, WO2017/070613, WO2017/070620, WO2017/099823, and WO2017/112865. In that context, the disclosures of WO2009/086558, WO2009/127060, WO2010/048536, WO2010/054406, WO2010/088537, WO2010/129709, WO2011/153493, US2011/0256175, US2012/0128760, US2012/0027803, US8158601, WO2016/118724, WO2016/118725, WO2017/070613, WO2017/070620, WO2017/099823, and WO2017/112865 specifically relating to (cationic) lipids suitable for LNPs are incorporated herewith by reference.

In some embodiments, the lipid is selected from the group consisting of 98N12-5, C12-200, and ckk-E12.

In some embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention.

In some embodiments, the protonatable lipids have a pKa of the protonatable group in the range of about 4 to about 11, e.g., a pKa of about 5 to about 7.

LNPs can comprise two or more (different) cationic lipids. The cationic lipids may be selected to contribute different advantageous properties. E.g., cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the RNA which is used as cargo. The N/P ratio may be calculated on the basis that e.g. lug RNA typically contains about 3 nmo1 phosphate residues, provided that the RNA exhibits a statistical distribution of bases. The “N”-value of the lipid or lipidoid may be calculated on the basis of its molecular weight and the relative content of permanently cationic and—if present—cationisable groups.

LNP in vivo characteristics and behavior can be modified by addition of a hydrophilic polymer coating, e.g. polyethylene glycol (PEG), to the LNP surface to confer steric stabilization. Furthermore, LNPs can be used for specific targeting by attaching ligands (e.g. antibodies, peptides, and carbohydrates) to its surface or to the terminal end of the attached PEG chains (e.g. via PEGylated lipids or PEGylated cholesterol).

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyI]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as w-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In preferred embodiments of the second aspect, the at least one RNA is complexed with one or more lipids thereby forming LNPs, wherein the LNP additionally comprises a PEGylated lipid with the formula (IV):

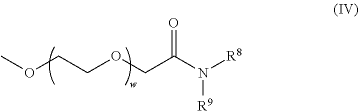

or a pharmaceutically acceptable salt, tautomer or stereoisomer thereof, wherein R⁸ and R⁹ are each independently a straight or branched, saturated or unsaturated alkyl chain containing from 10 to 30 carbon atoms, wherein the alkyl chain is optionally interrupted by one or more ester bonds; and w has mean value ranging from 30 to 60.

In some of the foregoing embodiments of the PEGylated lipid according to formula (IV), R⁸ and R⁹ are not both n-octadecyl when w is 42. In some other embodiments, R⁸ and R⁹ are each independently a straight or branched, saturated or unsaturated alkyl chain containing from 10 to 18 carbon atoms. In some embodiments, R⁸ and R⁹ are each independently a straight or branched, saturated or unsaturated alkyl chain containing from 12 to 16 carbon atoms. In some embodiments, R⁸ and R⁹ are each independently a straight or branched, saturated or unsaturated alkyl chain containing 12 carbon atoms. In some embodiments, R⁸ and R⁹ are each independently a straight or branched, saturated or unsaturated alkyl chain containing 14 carbon atoms. In other embodiments, R⁸ and R⁹ are each independently a straight or branched, saturated or unsaturated alkyl chain containing 16 carbon atoms. In still more embodiments, R⁸ and R⁹ are each independently a straight or branched, saturated or unsaturated alkyl chain containing 18 carbon atoms. In still other embodiments, R⁸ is a straight or branched, saturated or unsaturated alkyl chain containing 12 carbon atoms and R⁹ is a straight or branched, saturated or unsaturated alkyl chain containing 14 carbon atoms.

In various embodiments, w spans a range that is selected such that the PEG portion of the PEGylated lipid according to formula (IV) has an average molecular weight of about 400 to about 6000 g/mol. In some embodiments, the average w is about 50.

In preferred embodiments of the second aspect, R⁸ and R⁹ of the PEGylated lipid according to formula (IV) are saturated alkyl chains.

In a particularly preferred embodiment of the second aspect, the at least one RNA is complexed with one or more lipids thereby forming LNPs, wherein the LNP additionally comprises a PEGylated lipid, wherein the PEG lipid is of formula (IVa)

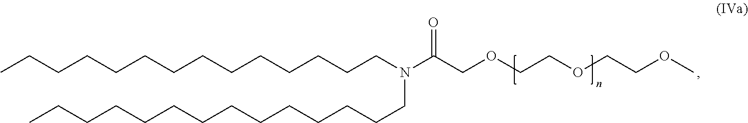

wherein n has a mean value ranging from 30 to 60, such as about 28 to about 32, about 30 to about 34, 32 to about 36, about 34 to about 38, 36 to about 40, about 38 to about 42, 40 to about 44, about 42 to about 46, 44 to about 48, about 46 to about 50, 48 to about 52, about 50 to about 54, 52 to about 56, about 54 to about 58, 56 to about 60, about 58 to about 62. In preferred embodiments, n is about 45, 46, 47, 48, 49, 50, 51, 52, 53, 54. In a most preferred embodiment n has a mean value of 49.

In other embodiments, the PEGylated lipid has one of the following structures:

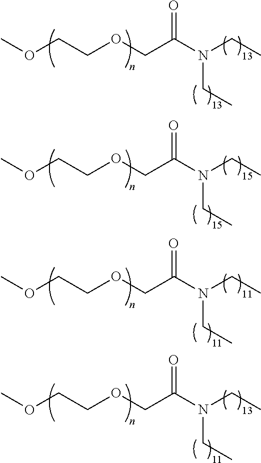

wherein n is an integer selected such that the average molecular weight of the PEGylated lipid is about 2500g/mol, most preferably n is about 49.

Further examples of PEG-lipids suitable in that context are provided in US2015/0376115A1 and WO2015/199952, each of which is incorporated by reference in its entirety.

In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2,5%, about 2%, about 1.5%, about 1%, about 0.5%, or about 0.3% on a molar basis (based on 100% total moles of lipids in the LNP). In preferred embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP). In various embodiments, the molar ratio of the cationic lipid to the PEGylated lipid ranges from about 100:1 to about 25:1.

In preferred embodiments, the LNP additionally comprises one or more additional lipids which stabilize the formation of particles during their formation (e.g. neutral lipid and/or one or more steroid or steroid analogue).

In preferred embodiments of the second aspect, the RNA is complexed with one or more lipids thereby forming LNPs, wherein the LNP additionally comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceram ides, sphingomyelins, dihydro sphingomyelins, cephalins, and cerebrosides.

In embodiments of the second aspect, the LNP additionally comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl- phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE).

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In preferred embodiments of the second aspect, the neutral lipid is 1,2-distearoyl-sn-glycero-3-phosphocholine (DSPC). The molar ratio of the cationic lipid to DSPC may be in the range from about 2:1 to 8:1.

In preferred embodiments of the second aspect, the steroid is cholesterol. The molar ratio of the cationic lipid to cholesterol may be in the range from about 2:1 to 1:1.

In some embodiments, the cholesterol may be PEGylated.

The sterol can be about 10 mol % to about 60 mol % or about 25 mol % to about 40 mol % of the lipid particle.

In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the LNP).

Preferably, LNPs comprise: (a) at least one RNA, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the LNPs comprise a lipid of formula (III), at least one RNA as defined herein, a neutral lipid, a steroid and a PEGylated lipid. In preferred embodiments, the lipid of formula (III) is lipid compound III-3, the neutral lipid is DSPC, the steroid is cholesterol, and the PEGylated lipid is the compound of formula (IVa).

In a preferred embodiment of the second aspect, the LNP consists essentially of (i) at least one cationic lipid; (ii) a neutral lipid; (iii) a sterol, e.g. , cholesterol; and (iv) a PEG-lipid, e.g. PEG-DMG or PEG-cDMA, in a molar ratio of about 20-60% cationic lipid: 5-25% neutral lipid: 25-55% sterol; 0.5-15% PEG-lipid.

In particularly preferred embodiments of the second aspect, the at least one RNA is complexed with one or more lipids thereby forming LNPs, wherein the LNP essentially consists of
(i) at least one cationic lipid as defined herein, preferably a lipid of formula (III), more preferably lipid III-3;
(ii) a neutral lipid as defined herein, preferably 1,2-distearoyl-sn-glycero-3-phosphocholine (DSPC);
(iii) a steroid or steroid analogue as defined herein, preferably cholesterol; and
(iv) a PEG-lipid as defined herein, e.g. PEG-DMG or PEG-cDMA, preferably a PEGylated lipid of formula (IVa),
wherein (i) to (iv) are in a molar ratio of about 20-60% cationic lipid: 5-25% neutral lipid: 25-55% sterol; 0.5-15% PEG-lipid.

In one preferred embodiment, the LNP comprises: a cationic lipid with formula (III) and/or PEG lipid with formula (IV), optionally a neutral lipid, preferably 1,2-distearoyl-sn-glycero-3-phosphocholine (DSPC) and optionally a steroid, preferably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In a particular preferred embodiment, the composition of the second aspect comprising at least one RNA of the first aspect comprises LNPs, which have a molar ratio of approximately 50:10:38.5:1.5, preferably 47.5:10:40.8:1.7 or more preferably 47.4:10:40.9:1.7 (i.e. proportion (mol%) of cationic lipid (preferably lipid III-3), DSPC, cholesterol and PEG-lipid ((preferably PEG-lipid of formula (IVa) with n =49); solubilized in ethanol).

The total amount of RNA in the LNPs may vary and is defined depending on the e.g. RNA to total lipid w/w ratio. In one embodiment of the invention the RNA to total lipid ratio is less than 0.06 w/w, preferably between 0.03 w/w and 0.04 w/w.

In various embodiments, the LNP as defined herein have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average as determined by dynamic light scattering as commonly known in the art.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In embodiments where more than one or a plurality, e.g. 2, 3, 4, 5, 6, 7, 8, 9, 10, 11, 12, 13, 14, 15 of the RNAs of the first aspect are comprised in the composition, said more than one or said plurality e.g. 2, 3, 4, 5, 6, 7, 8, 9, 10, 11, 12, 13, 14, 15 of the RNAs may be complexed within one or more lipids thereby forming LNPs comprising more than one or a plurality, e.g. 2, 3, 4, 5, 6, 7, 8, 9, 10, 11, 12, 13, 14, 15 of different RNAs.

According to further embodiments, the composition of the second aspect may comprise at least one adjuvant. Suitably, the adjuvant is preferably added to enhance the immunostimulatory properties of the composition.

The term “adjuvant” as used herein will be recognized and understood by the person of ordinary skill in the art, and is e.g. intended to refer to an agent that may modify, e.g. enhance, the effect of other agents (herein: the effect of the RNA) or that may be suitable to support administration and delivery of the composition. The term “adjuvant” refers to a broad spectrum of substances. Typically, these substances are able to increase the immunogenicity of antigens (herein: antigen is a product of translation provided by the RNA). For example, adjuvants may be recognized by the innate immune systems and, e.g., may elicit an innate immune response. “Adjuvants” typically do not elicit an adaptive immune response. In the context of the invention, adjuvants may enhance the effect of the antigenic peptide or protein provided by the RNA as defined herein. In that context, the at least one adjuvant may be selected from any adjuvant known to a skilled person and suitable for the present case, i.e. supporting the induction of an immune response in a subject.

Accordingly, the composition of the second aspect may comprise at least one adjuvant, wherein the at least one adjuvant may be suitably selected from any adjuvant provided in WO2016/203025. Adjuvants disclosed in any of the claims 2 to 17 of WO2016/203025, preferably adjuvants disclosed in claim 17 of W02016/203025 are particularly suitable, the specific content relating thereto herewith incorporated by reference.

The composition of the second aspect may comprise, besides the components specified herein, at least one further component which may be selected from the group consisting of further antigens (e.g. in the form of a peptide or protein) or further antigen-encoding nucleic acids; a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and/or an adjuvant nucleic acid, preferably an immunostimulatory RNA (isRNA), e.g. CpG-RNA etc.

Vaccine:

In a third aspect, the present invention provides a vaccine comprising the RNA of the first aspect or the composition of the second aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect.

The term “vaccine” will be recognized and understood by the person of ordinary skill in the art, and is e.g. intended to be a prophylactic or therapeutic material providing at least one epitope or antigen, preferably an immunogen. In the context of the invention the antigen or antigenic function is provided by the inventive RNA of the first aspect, or the composition of the second aspect (comprising at least one RNA of the first aspect)

In preferred embodiments of the third aspect, the vaccine comprises the RNA of the first aspect, the composition of the second aspect wherein said RNA or said composition elicits an adaptive immune response.

In particularly preferred embodiments, the vaccine comprises the RNA of the first aspect or the composition of the second aspect wherein said RNA or said composition elicits an adaptive immune response, preferably an adaptive immune response against LASV.

According to a preferred embodiment of the third aspect, the vaccine as defined herein may further comprise a pharmaceutically acceptable carrier and optionally at least one adjuvant as specified in the context of the second aspect.

Suitable adjuvants in that context may be selected from adjuvants disclosed in claim 17 of WO2016/203025.

In a preferred embodiment, the vaccine is a monovalent vaccine.

In embodiments the vaccine is a polyvalent vaccine comprising a plurality or at least more than one of the RNAs as defined in the context of the first aspect. Embodiments relating to a polyvalent composition as disclosed in the context of the second aspect may likewise be read on and be understood as suitable embodiments of the polyvalent vaccine of the third aspect. In a preferred embodiment, the vaccine is a tetravalent vaccine

Said tetravalent vaccine may suitably comprise one RNA species encoding a GPC or prefusion-stabilized GPC construct, wherein the GPC or prefusion-stabilized GPC is or is derived from a clade I LASV; one RNA species encoding a GPC or prefusion-stabilized GPC construct, wherein the GPC or prefusion-stabilized GPC is or is derived from a clade II LASV, one RNA species encoding a GPC or prefusion-stabilized GPC construct, wherein the GPC or prefusion-stabilized GPC is or is derived from a clade III LASV, and one RNA species encoding a GPC or prefusion-stabilized GPC construct, wherein the GPC or prefusion-stabilized GPC is or is derived from a clade IV LASV.

Embodiments relating to a tetravalent composition as described in the context of the second aspect may likewise be read on and be understood as suitable embodiments of the tetravalent vaccine.

The vaccine of the third aspect typically comprises a safe and effective amount of the RNA as specified herein. As used herein, “safe and effective amount” means an amount of the RNA that is sufficient to significantly induce a positive modification of a disease or disorder related to an infection with a LASV. At the same time, a “safe and effective amount” is small enough to avoid serious side-effects. In relation to the vaccine or composition of the present invention, the expression “safe and effective amount” preferably means an amount of the RNA that is suitable for stimulating the adaptive immune system in such a manner that no excessive or damaging immune reactions are achieved but, preferably, also no such immune reactions below a measurable level.

A “safe and effective amount” of the RNA of the composition or vaccine as defined above will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying medical doctor. Moreover, the “safe and effective amount” of the RNA, the composition, the vaccine may depend from application route (intradermal, intramuscular), application device (jet injection, needle injection, microneedle patch) and/or complexation (protamine complexation or LNP encapsulation). Moreover, the “safe and effective amount” of the RNA, the composition, the vaccine may depend from the condition of the treated subject (infant, pregnant women, immunocompromised human subject etc.). Accordingly, the suitable “safe and effective amount” has to be adapted accordingly and will be chosen and defined by the skilled person.

The vaccine can be used according to the invention for human medical purposes and also for veterinary medical purposes (mammals, vertebrates, avian species), as a pharmaceutical composition, or as a vaccine.

In a preferred embodiment, the RNA, the composition, or the vaccine is provided in lyophilized form (using e.g. lyophilisation methods as described in WO2016/165831, WO2011/069586, WO2016/184575 or WO2016/184576). Preferably, the lyophilized RNA, the lyophilized composition, or the lyophilized vaccine is reconstituted in a suitable buffer, advantageously based on an aqueous carrier, prior to administration, e.g. Ringer-Lactate solution or a phosphate buffer solution.

Accordingly, the pharmaceutically acceptable carrier as used herein preferably includes the liquid or non-liquid basis of the inventive vaccine. If the inventive vaccine is provided in liquid form, the carrier will be water, typically pyrogen-free water; isotonic saline or buffered (aqueous) solutions, e.g. phosphate, citrate etc. buffered solutions. Preferably, Ringer-Lactate solution is used as a liquid basis for the vaccine or the composition according to the invention as described in WO2006/122828, the disclosure relating to suitable buffered solutions incorporated herewith by reference.

The choice of a pharmaceutically acceptable carrier as defined herein may be determined by the manner in which the vaccine is administered. The vaccine or composition may be administered, e.g., systemically or locally. Routes for systemic administration in general include, e.g., transdermal, oral, parenteral routes, including subcutaneous, intravenous, intramuscular, intra-arterial, intradermal and intraperitoneal injections and/or intranasal administration routes. Routes for local administration in general include, e.g., topical administration routes but also intradermal, transdermal, subcutaneous, or intramuscular injections or intralesional, intracranial, intrapulmonal, intracardial, intraarticular and sublingual injections. More preferably, compositions or vaccines according to the present invention may be administered by an intradermal, subcutaneous, or intramuscular route, preferably by injection, which may be needle-free and/or needle injection. Compositions/vaccines are therefore preferably formulated in liquid or solid form. The suitable amount of the vaccine or composition according to the invention to be administered can be determined by routine experiments, e.g. by using animal models (e.g. rabbit, ferret, sheep, mouse, rat, dog and non-human primate). Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition or vaccine is to be administered perorally, tablets, capsules and the like are the preferred unit dose form. The pharmaceutically acceptable carriers for the preparation of unit dose forms which can be used for oral administration are well known in the prior art.

The inventive vaccine or composition as defined herein can additionally comprise one or more auxiliary substances as defined above in order to further increase the immunogenicity. A synergistic action of the RNA contained in the vaccine or compositionand of an auxiliary substance, which may be optionally be co- formulated (or separately formulated) with the inventive vaccine or composition as described above, is preferably achieved thereby. Such immunogenicity increasing agents or compounds may be provided separately (not co-formulated with the inventive vaccine or composition) and administered individually.

Further additives which may be included in the vaccine or composition are emulsifiers, such as for example, tween; wetting agents, such as, for example, sodium lauryl sulfate; colouring agents; taste-imparting agents, pharmaceutical carriers; tablet-forming agents; stabilizers; antioxidants; preservatives.

Kit or Kit of Parts, Application, Medical Uses, Method of Treatment:

In a fourth aspect, the present invention provides a kit or kit of parts, wherein the kit or kit of parts comprises the RNA of the first aspect, the composition of the second aspect, and/or the vaccine of the third aspect, optionally comprising a liquid vehicle for solubilising, and optionally technical instructions providing information on administration and dosage of the components.

In a preferred embodiment, the kit or kit of parts of the fourth aspect comprises at least the following components
a) at least one RNA of the first aspect, preferably encoding at least one antigenic peptide or protein derived from a LASV GPC, most preferably an RNA selected from Table 2 or Table 3.13, wherein said RNA is preferably complexed with one or more lipids thereby forming lipid nanoparticles (LNP); and
b) at least one, two, or three further RNA encoding an antigenic peptide or protein derived from LASV NP or LASV Z, most preferably an RNA selected from Table 4, 4.1-4.3 or 5, wherein said further RNA is preferably complexed with one or more lipids thereby forming lipid nanoparticles (LNP),
wherein components a) and b) are provided as separate entities or as a single entity.

In a preferred embodiment, the kit or kit of parts of the fourth aspect comprises at least the following components
c) at least one RNA of the first aspect, preferably encoding at least one antigenic peptide or protein derived from a LASV prefusion-stabilized GPC, most preferably an RNA selected from Table 3.1-3.12, wherein said RNA is preferably complexed with one or more lipids thereby forming lipid nanoparticles (LNP); and
d) at least one, two, or three further RNA encoding an antigenic peptide or protein derived from LASV NP or LASV Z, most preferably an RNA selected from Table 4, 4.1-4.3 or 5, wherein said further RNA is preferably complexed with one or more lipids thereby forming lipid nanoparticles (LNP), wherein components a) and b) are provided as separate entities or as a single entity.

In a preferred embodiment, the kit or kit of parts of the fourth aspect comprises at least the following components
a) at least one RNA of the first aspect, preferably encoding at least one antigenic peptide or protein derived from a LASV GPC or prefusion-stabilized GPC, wherein said RNA is preferably complexed with one or more lipids thereby forming lipid nanoparticles (LNP); and
b) at least one further RNA encoding an antigenic peptide or protein derived from LASV NP (or SP-NP), wherein said further RNA is preferably complexed with one or more lipids thereby forming lipid nanoparticles (LNP); and
c) at least one further RNA encoding an antigenic peptide or protein derived from LASV Z, wherein said further RNA is preferably complexed with one or more lipids thereby forming lipid nanoparticles (LNP), wherein components a), b) and c) are provided as separate entities or as a single entity.

The antigenic peptide(s) or protein(s) may be derived from different LASV clades, in particular from clade I, II, III, IV, V, or VI and/or from a LASV of lineage I, II, III, IV, V, or VI (see List 1-6 and Table A). Such embodiments may have the advantage that the components of the kit or kit of parts, when administered to the subject, provide broad protection against different LASV clades which is important in the context of an effective LASV vaccine.

The kit or kit of parts may further comprise additional components as described in the context of the composition of the second aspect or the vaccine of the third aspect.

The technical instructions of said kit or kit of parts may comprise information about administration and dosage and patient groups. Such kits, preferably kits of parts, may be applied e.g. for any of the applications or uses mentioned herein, preferably for the use of the RNA of the first aspect, the composition of the second aspect, the vaccine of the third aspect, for the treatment or prophylaxis of an infection or diseases caused by LASV or disorders related thereto. Preferably, the RNA of the first aspect, the composition of the second aspect, the vaccine of the third aspect is provided in a separate part of the kit, wherein the RNA of the first aspect, the composition of the second aspect, or the vaccine of the third aspect is preferably lyophilised. The kit may further contain as a part a vehicle (e.g. buffer solution) for solubilising the RNA of the first aspect, the composition of the second aspect, or the vaccine of the third aspect.

In preferred embodiments, the kit or kit of parts as defined herein comprises Ringer lactate solution.

Any of the above kits may be used in a treatment or prophylaxis as defined herein. More preferably, any of the above kits may be used as a vaccine, preferably a vaccine against infections caused by LASV as defined herein.

Medical Use:

In a further aspect, the present invention relates to the first medical use of the RNA of the first aspect, the composition of the second aspect, the vaccine of the third aspect, or the kit or kit of parts of the fourth aspect.

Accordingly, the RNA of the first aspect, the composition of the second aspect, the vaccine of the third aspect, or the kit or kit of parts of the fourth aspect is for use as a medicament.

The present invention furthermore provides several applications and uses of the RNA of the first aspect, the composition of the second aspect, the vaccine of the third aspect, or the kit or kit of parts of the fourth aspect.

In particular, said RNA, composition, vaccine, kit or kit of parts may be used for human medical purposes and also for veterinary medical purposes, preferably for human medical purposes.

In another aspect, the present invention relates to the second medical use of the RNA of the first aspect, the composition of the second aspect, the vaccine of the third aspect, or the kit or kit of parts of the fourth aspect.

Accordingly, the RNA of the first aspect, the composition of the second aspect, the vaccine of the third aspect, or the kit or kit of parts of the fourth aspect is for use in the treatment or prophylaxis of an infection with LASV, or a disorder related to such an infection.

In particular, the RNA of the first aspect, the composition of the second aspect, the vaccine of the third aspect, or the kit or kit of parts of the fourth aspect may be used in the treatment or prophylaxis of an infection with LASV, or a disorder related to such an infection for human and also for veterinary medical purposes, preferably for human medical purposes.

As used herein, “a disorder related to a LASV infection” may preferably comprise a typical symptom or a complication of an LASV infection.

Particularly, the RNA of the first aspect, the composition of the second aspect, the vaccine of the third aspect, or the kit or kit of parts of the fourth aspect may be used in a method of prophylactic (pre-exposure prophylaxis or post-exposure prophylaxis) and/or therapeutic treatment of infections caused by LASV.

The composition, vaccine, or the kit or kit of parts as defined herein may preferably be administered locally, in particular, by an intradermal, subcutaneous, intranasal, or intramuscular route. Inventive compositions or vaccines of the invention are therefore preferably formulated in liquid (or sometimes in solid) form. In embodiments, composition, vaccine, or the kit or kit of parts may be administered by conventional needle injection or needle-free jet injection. Preferred in that context is the RNA, the composition, the vaccine is administered by intramuscular needle injection.

The term “jet injection”, as used herein, refers to a needle-free injection method, wherein a fluid (composition, vaccine,) containing e.g. at least one RNA of the first aspect is forced through an orifice, thus generating an ultra-fine liquid stream of high pressure that is capable of penetrating mammalian skin and, depending on the injection settings, subcutaneous tissue or muscle tissue. In principle, the liquid stream perforates the skin, through which the liquid stream is pushed into the target tissue. Preferably, jet injection is used for intradermal, subcutaneous or intramuscular injection of the RNA, the compositions, the vaccines disclosed herein.

In embodiments, the RNA as comprised in a composition, vaccine, kit or kit of parts as defined herein is provided in an amount of about 100 ng to about 500 ug, about 1 ug to about 200 ug, about 1 ug to about 100 ug, about 5 ug to about 100 ug, about 10 ug to about 50 ug, specifically, in an amount of about 5 ug, 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug.

Depending from application route (intradermal, intramuscular, intranasal), application device (jet injection, needle injection, microneedle patch) and/or complexation (preferably LNP encapsulation) the suitable amount has to be adapted accordingly and will be chosen and defined by the skilled person.

The immunization protocol for the treatment or prophylaxis of an infection as defined herein, i.e. the immunization of a subject against a LASV, typically comprises a series of single doses or dosages of the composition or the vaccine. A single dosage, as used herein, refers to the initial/first dose, a second dose or any further doses, respectively, which are preferably administered in order to “boost” the immune reaction.

In one embodiment, the immunization protocol for the treatment or prophylaxis of an infection as defined herein, i.e. the immunization of a subject against a LASV, comprises one single doses of the composition or the vaccine.

The treatment or prophylaxis as defined above may comprise the administration of a further active pharmaceutical ingredient.

For example, at least one LASV protein or peptide as described herein, or a fragment or variant thereof, may be co-administered in order to induce or enhance an immune response. Further, two distinct RNAs of the first aspect and, optionally further RNAs of the first aspect may be administered at different time points, preferably in a prime-boost scenario, e.g. using a composition comprising at least one LASV polypeptide as prime vaccination and a composition/vaccine comprising at least one RNA of the first aspect as boost vaccination.

Suitably, the treatment or prophylaxis as defined above comprises the administration of a further active pharmaceutical ingredient, wherein the further active pharmaceutical ingredient may be an immunotherapeutic agent that can be selected from immunoglobulins, preferably IgGs, monoclonal or polyclonal antibodies, polyclonal serum or sera, etc., most preferably immunoglobulins directed against a LASV protein or peptide as defined herein. Preferably, such a further immunotherapeutic agent may be provided as a peptide/protein or may be encoded by a nucleic acid, preferably by a DNA or an RNA, more preferably an mRNA.

Method of Treatment and Use, Diagnostic Method and Use:

In another aspect, the present invention relates to a method of treating or preventing a disorder.

In preferred embodiments, the present invention relates to a method of treating or preventing a disorder, wherein the method comprises applying or administering to a subject in need thereof the RNA of the first aspect, the composition of the second aspect, the vaccine of the third aspect, or the kit or kit of parts of the fourth aspect.

In preferred embodiments, the disorder is an infection with LASV or a disorder related to such an infection.

In preferred embodiments, the present invention relates to a method of treating or preventing a disorder, wherein the method comprises applying or administering to a subject in need thereof the RNA of the first aspect, the composition of the second aspect, the vaccine of the third aspect, or the kit or kit of parts of the fourth aspect, wherein the subject in need is preferably a mammalian subject. In particularly preferred embodiments, the mammalian subject is a human subject.

In particular, such a method may preferably comprise the steps of:
a) providing the RNA of the first aspect, the composition of the second aspect, the vaccine of the third aspect, or the kit or kit of parts of the fourth aspect;
b) applying or administering said RNA, composition, vaccine or kit or kit of parts of the to a tissue or an organism;
c) optionally, administering immunoglobulin (IgGs) against a LASV;
d) optionally, administering a further substance (adjuvant, auxiliary substance, further antigen, further RNA).

According to a further aspect, the present invention also provides a method for expression of at least one polypeptide comprising at least one peptide or protein derived from a LASV, or a fragment or variant thereof, wherein the method preferably comprises the following steps:
a) providing the RNA of the first aspect or the composition of the second aspect; and
b) applying or administering said RNA or composition to an expression system (cells), a tissue, an organism.

The method may be applied for laboratory, for research, for diagnostic, for commercial production of peptides or proteins and/or for therapeutic purposes. The method may furthermore be carried out in the context of the treatment of a specific disease, particularly in the treatment of infectious diseases, particularly LASV infections.

Likewise, according to another aspect, the present invention also provides the use of the RNA of the first aspect, the composition of the second aspect, the vaccine of the third aspect, or the kit or kit of parts of the fourth aspect preferably for diagnostic or therapeutic purposes, e.g. for expression of an encoded LASV antigenic peptide or protein, e.g. by applying or administering said RNA, composition comprising said RNA, vaccine comprising said RNA, e.g. to a cell-free expression system, a cell (e.g. an expression host cell or a somatic cell), a tissue or an organism. In specific embodiments, applying or administering said RNA, composition comprising said RNA, vaccine comprising said RNA to a tissue or an organism is followed by e.g. a step of obtaining induced LASV GPC antibodies e.g. LASV GPC specific (monoclonal) antibodies.

The use may be applied for a (diagnostic) laboratory, for research, for diagnostics, for commercial production of peptides, proteins, or LASV antibodies and/or for therapeutic purposes. The use may be carried out in vitro, in vivo or ex vivo. The use may furthermore be carried out in the context of the treatment of a specific disease, particularly in the treatment of an LASV infection or a related disorder.

In a particularly preferred embodiment, the invention provides the RNA of the first aspect, the composition of the second aspect, the composition of the third aspect, the vaccine of the fourth aspect, or the kit or kit of parts of the fifth aspect for use as a medicament, for use in treatment or prophylaxis, preferably treatment or prophylaxis of an LASV infection or a related disorder, or for use as a vaccine.

According to a further aspect, the present invention also provides a method of manufacturing a composition or a LASV vaccine, comprising the steps of:
a) RNA in vitro transcription step using a DNA template in the presence of a trinuclotide cap analogue to obtain cap1 comprising coding RNA,
b) Purifying the obtained cap1 comprising coding RNA of step a) using RP-HPLC, and/or TFF, and/or Oligo(dT) purification and/or AEX, preferably using RP-HPLC;
c) Providing a first liquid composition comprising the purified cap1 comprising coding RNA of step b);
d) Providing a second liquid composition comprising at least one cationic lipid as defined herein, a neutral lipid as defined herein, a steroid or steroid analogue as defined herein, and a PEG-lipid as defined herein;
e) Introducing the first liquid composition and the second liquid composition into at least one mixing means to allow the formation of LN Ps comprising cap1 comprising coding RNA;
f) Purifying the obtained LNPs comprising cap1 comprising coding RNA;
g) optionally, lyophilizing the purified LNPs comprising cap1 comprising coding RNA.

Preferably, the mixing means of step e) is a T-piece connector or a microfluidic mixing device. Preferably, the purifying step f) comprises at least one step selected from precipitation step, dialysis step, filtration step, TFF step. Optionally, an enzymatic polyadenylation step may be performed after step a) or b). Optionally, further purification steps may be implemented to e.g. remove residual DNA, buffers, small RNA by-products etc. Optionally, RNA in vitro transcription is performed in the absence of a cap analog, and an enzymatic capping step is performed after RNA vitro transcription. Optionally, RNA in vitro transcription is performed in the presence of at least one modified nucleotide as defined herein.

List of Preferred Embodiments/Items

In the following, particularly preferred embodiments (items 1-49) of the invention are provided.

Item 1:

RNA comprising at least one coding sequence encoding at least one antigenic peptide or protein derived from a Lassa virus (LASV) protein or a fragment or variant thereof, wherein said coding sequence is operably linked to a 5′-UTR derived from a HSD17B4 gene, a NDUFA4 gene, or a RPL32 gene and/or a 3′-UTR derived from a PSMB3 gene, a CASP1 gene, an ALB7 gene, or an alpha-globin gene.

Item 2:

RNA according to item 1, wherein said coding sequence is operably linked to a 5′-UTR and/or 3′-UTR, comprising
a-1. at least one 5′-UTR derived from a 5′-UTR of a HSD17B4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-2. at least one 5′-UTR derived from a 5′-UTR of a NDUFA4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
b-4. at least one 5′-UTR derived from a 5′-UTR of a HSD17B4 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
i-2. at least one 5′-UTR derived from a 5′-UTR of a RPL32 gene, or from a corresponding RNA sequence, homolog, fragment or variant thereof and at least one 3′-UTR derived from a 3′-UTR of a ALB7 gene, or from a corresponding RNA sequence, homolog, fragment or variant thereof; or
i-3. at least one 3′-UTR derived from a 3′-UTR of a alpha-globin gene gene, or from a corresponding RNA sequence, homolog, fragment or variant thereof.

Item 3:

RNA according to item 1 or 2, wherein
said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13804 or 13805 or a fragment or a variant thereof;
said 5′-UTR derived from a NDUFA4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13812 or 13813 or a fragment or a variant thereof;
said 5′-UTR derived from a RPL32 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13824 or 13825 or a fragment or a variant thereof;
said 3′-UTR derived from a PSMB3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13826 or 13827 or a fragment or a variant thereof;
said 3′-UTR derived from a CASP1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13828 or 13829 or a fragment or a variant thereof;
said 3′-UTR derived from a ALB7 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13838 or 13839 or a fragment or a variant thereof;
said 3′-UTR derived from a alpha-globin gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to EQ ID NO: 13840 or 13841 or a fragment or a variant thereof.

Item 4:

RNA according to any one of the preceding items, wherein the LASV protein is derived from glycoprotein precursor (GPC), a prefusion-stabilized GPC, nucleoprotein (NP), zinc-binding matrix protein (Z), or a variant, fragment, or combination thereof, wherein GPC, prefusion-stabilized GPC, NP, Z are preferably full-length proteins.

Item 4.1:

RNA according to item 4, wherein the LASV protein is derived from glycoprotein precursor (GPC), wherein the GPC or prefusion-stabilized GPC is C-terminally truncated, preferably lacking the cytoplasmic tail (herein referred to as GPCmut13, or GPCmut14 to GPCmut25).

Item 4.2:

RNA according to item 4, wherein the LASV protein is derived from nucleoprotein (NP), additionally encoding a heterologous secretory signal peptide, preferably derived from tissue plasminogen activator (TPA or HsPLAT), human serum albumin (HSA or HsALB), or immunoglobulin IgE (IgE) (herein referred to as SP-NP or SP-HsPLAT_NP, SP-HsALB_NP, SP-IgE_NP).

Item 4.3:

RNA according to item 4, wherein the LASV protein is preferably derived from glycoprotein precursor (GPC) or from prefusion-stabilized GPC and is mutated/substituted to delete at least one predicted or potential glycosylation site.

Item 5:

RNA according item 4, wherein the prefusion-stabilized GPC comprises at least one of the following mutations
A, preferably of Al, A2, A3 and B, and C:
A amino acid substitutions allowing a covalent link of different structural elements of GPC, preferably by introduction of two additional cysteine residues, wherein preferably
   A1 the amino acid substitutions allowing a covalent link of GP1 and GP2, preferably by introduction of two additional cysteine residues, preferably at position 207 or 206 and 360 or 359, preferably by amino acid substitutions R2070 or R2060 and G3600 or G359C, or
   A2 the amino acid substitutions allowing a covalent link of GP1 to HR1 of GP2, preferably by introduction of two additional cysteine residues, preferably at position 81 or 80 and 319 or 318, preferably by amino acid substitutions T81C or T800 and N3190 or N3180, or
   A3 the amino acid substitutions allowing a covalent link of T-loop in GP2 to adjacent loop in GP2, preferably by introduction of two additional cysteine residues, preferably at positon 370 or 369 and 386 or 385, preferably by amino acid substitutions W3700 or W369C and W386C or W385C,
B amino acid substitutions allowing stabilization of GP2, preferably by introduction of a helix- breaking point mutation in the metastable region of HR1 of GP2 subunit, more preferably by introducing E329P or E328P,
C replacing the native GP1-GP2 cleavage site (S1 P) with a furin cleavage site, e.g. RRLL to RRRR to facilitate efficient processing of the mRNA encoded antigenic protein in target cells, preferably by amino acid substitutions L258R, L259R or L257R, L258R,

wherein prefusion-stabilized GPC preferably comprises at least one mutation A selected from A1, A2 and A3 (herein referred to as GPCmut1, GPCmut2, GPCmut3, GPCmut4, GPCmut5, GPCmut6, GPCmut7, GPCmut8, GPCmut9, GPCmut10, GPCmut11, or GPCmut12).

Item 6:

RNA according to any one of the preceding items, wherein
said at least one coding sequence encodes at least one antigenic peptide or protein derived from LASV GPC comprising or consisting of an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 1-254 or a fragment or a variant thereof;
said at least one coding sequence encoding at least one antigenic peptide or protein derived from LASV prefusion-stabilized GPC (GPCmutl or GPCstabilized) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 3567-3820 or a fragment or variant of any of these sequences;
said at least one coding sequence encoding at least one antigenic peptide or protein derived from LASV prefusion-stabilized GPC (GPCmut2) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 22949-22980 or a fragment or variant of any of these sequences;
said at least one coding sequence encoding at least one antigenic peptide or protein derived from LASV prefusion-stabilized GPC (GPCmut3) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 24645-24676 or a fragment or variant of any of these sequences;
said at least one coding sequence encoding at least one antigenic peptide or protein derived from LASV prefusion-stabilized GPC (GPCmut4) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 26341-26372 or a fragment or variant of any of these sequences;
said at least one coding sequence encoding at least one antigenic peptide or protein derived from LASV prefusion-stabilized GPC (GPCmut5) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 28037-28068 or a fragment or variant of any of these sequences;
said at least one coding sequence encoding at least one antigenic peptide or protein derived from LASV prefusion-stabilized GPC (GPCmut6) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 29733-29764 or a fragment or variant of any of these sequences;
said at least one coding sequence encoding at least one antigenic peptide or protein derived from LASV prefusion-stabilized GPC (GPCmut7) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 31429-31460 or a fragment or variant of any of these sequences;
said at least one coding sequence encoding at least one antigenic peptide or protein derived from LASV prefusion-stabilized GPC (GPCmut8) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 33125-33156 or a fragment or variant of any of these sequences;
said at least one coding sequence encoding at least one antigenic peptide or protein derived from LASV prefusion-stabilized GPC (GPCmut9) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 34821-34852 or a fragment or variant of any of these sequences;
said at least one coding sequence encoding at least one antigenic peptide or protein derived from LASV prefusion-stabilized GPC (GPCmut10) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 36517-36548 or a fragment or variant of any of these sequences;
said at least one coding sequence encoding at least one antigenic peptide or protein derived from LASV prefusion-stabilized GPC (GPCmut11) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 38213-38244 or a fragment or variant of any of these sequences,
said at least one coding sequence encoding at least one antigenic peptide or protein derived from LASV prefusion-stabilized GPC (GPCmut12) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 39909-39940 or a fragment or variant of any of these sequences,
said at least one coding sequence encoding at least one antigenic peptide or protein derived from LASV GPC lacking the cytoplasmic tail (GPCmut13) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 41605-41636 or a fragment or variant of any of these sequences,
said at least one coding sequence encodes at least one antigenic peptide or protein derived from LASV NP comprising or consisting of an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 7547-7797 or a fragment or a variant thereof;
said at least one coding sequence encoding at least one antigenic peptide or protein derived from HsPLAT_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 17968-18001 or a fragment or variant of any of these sequences;
said at least one coding sequence encoding at least one antigenic peptide or protein derived from HsALB_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 19328-19361 or a fragment or variant of any of these sequences;
said at least one coding sequence encoding at least one antigenic peptide or protein derived from IgE_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 20689-20722 or a fragment or variant of any of these sequences;
said at least one coding sequence encodes at least one antigenic peptide or protein derived from LASV Z comprising or consisting of an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 11166-11347 or a fragment or a variant thereof.

Item 7:

RNA according to any one of the preceding items, wherein
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 255-2286 or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 3821-6106 (encoding GPCmut1) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 22981-23108 (encoding GPCmut2) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 24677-24804 (encoding GPCmut3) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 26373-26500 (encoding GPCmut4) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 28069-28196 (encoding GPCmut5) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 29765-29892 (encoding GPCmut6) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 31461-31588 (encoding GPCmut7) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 33157-33284 (encoding GPCmut8) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 34853-34980 (encoding GPCmut9) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 36549-36676 (encoding GPCmut10) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 38245-38372 (encoding GPCmut11) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 39941-40068 (encoding GPCmut12) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 41637-41764 (encoding GPCmut13) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 7798-9805 (encoding NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 18002-18103 (encoding SP- HsPLAT_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 19362-19463 (encoding SP- HsALB_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 20723-20824 (encoding SP- IgE_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 11348-12803 (encoding Z) or a fragment or a fragment or variant of any of these sequences.

Item 8:

RNA according to any one of the preceding items, wherein the at least one coding sequence is a codon modified coding sequence, wherein the amino acid sequence encoded by the at least one codon modified coding sequence is preferably not being modified compared to the amino acid sequence encoded by the corresponding wild type coding sequence.

Item 9:

RNA according to item 8, wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

Item 10:

RNA according to item 8 or 9, wherein the
at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to any one SEQ ID NOs: 509-2286 (encoding GPC) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 4075-6106 (encoding GPCmut1) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 22981-23108 (encoding GPCmut2) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 24677-24804 (encoding GPCmut3) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 26373-26500 (encoding GPCmut4) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 28069-28196 (encoding GPCmut5) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 29765-29892 (encoding GPCmut6) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 31461-31588 (encoding GPCmut7) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 33157-33284 (encoding GPCmut8) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 34853-34980 (encoding GPCmut9) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 36549-36676 (encoding GPCmut10) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 38245-38372 (encoding GPCmut11) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 39941-40068 (encoding GPCmut12) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 41637-41764 (encoding GPCmut13) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 8049-9805 (encoding NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 18002-18103, 19362-19463, or 20723-20824 (encoding SP-NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 11530-12803 (encoding Z) or a fragment or variant of any of these sequences.

Item 11:

RNA according to item 8 to 10, wherein the
at least one coding sequence comprising a G/C optimized or G/C content modified coding sequence (opt1, opts, opt6, opt11) comprising a nucleic acid sequence which is identical or at least 50%, 60%, 70%, 80%, 85%, 86%, 87%, 88%, 89%, 90%, 91%, 92%, 93%, 94%, 95%, 96%, 97%, 98%, or 99% identical to any one of the nucleic acid sequences according to the SEQ ID NOs: 509-762, 1525-2286 (encoding GPC) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 4075-4582, 5345-6106 (encoding GPCmut1) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 22981-23044, 23077-23108 (encoding GPCmut2) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 24677-24740, 24773-24804 (encoding GPCmut3) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 26373-26436, 26469-26500 (encoding GPCmut4) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 28069-28132, 28165-28196 (encoding GPCmut5) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 29765-29828, 29861-29892 (encoding GPCmut6) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 31461-31524, 31557-31588 (encoding GPCmut7) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 33157-33220, 33253-33284 (encoding GPCmut8) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 34853-34916, 34949-34980 (encoding GPCmut9) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 36549-36612, 36645-36676 (encoding GPCmut10) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 38245-38308, 38341-38372 (encoding GPCmut11) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 39941-40004, 40037-40068 (encoding GPCmut12) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 41637-41700, 41733-41764 (encoding GPCmut13) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 8049-8299, 9053-9805 (encoding NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 18002-18035, 18070-18103, 19362-19395, 19430-19463, 20723-20756, 20791-20824 (encoding SP_NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 11530-11711, 12258-12803 (encoding Z) or a fragment or variant of any of these sequences.

Item 11.1:

RNA according to item 8 to 10, wherein the at least one coding sequence is a codon modified coding sequence, comprising at least one modified nucleotide selected from pseudouridine (ψ), N1-methylpseudouridine (m1ψ), 5-methylcytosine, and 5-methoxyuridine, wherein pseudouridine (ψ), and N1-methylpseudouridine (m1ψ) are particularly preferred.

Item 12:

RNA according to any one of the preceding items, wherein the RNA comprises a 5′-cap structure, preferably m7G(5′), m7G(5′)ppp(5′)(2′OMeA), or m7G(5′)ppp(5′)(2′OMeG), wherein cap1 (m7G(5′)ppp(5′)(2′OMeA), or m7G(5′)ppp(5′)(2′OMeG) is particularly preferred.

Item 13:

RNA according to any one of the preceding items, wherein the RNA comprises at least one poly(A) sequence, preferably comprising 30 to 200 adenosine nucleotides, preferably about 64 adenosine nucleotides (A64), about 100 adenosine nucleotides (A100) or about 150 adenosine nucleotides.

Item 13.1:

RNA according to any one of the preceding items, wherein the RNA comprises at least one poly(A) sequence, located (exactly) at the 3′ terminus of the coding RNA.

Item 14:

RNA according to any one of the preceding items, wherein the RNA comprises at least one histone stem-loop, wherein the histone stem-loop preferably comprises a nucleic acid sequence according to SEQ ID NOs: 13842 or 13843 or a fragment or variant thereof.

Item 15:

RNA according to any one of the preceding items comprising the following elements:
a) 5′-cap structure, preferably as defined in item 12;
b) 5′-UTR and/or 3′-UTR as defined in items 1 to 3;
c) at least one coding sequence, preferably as defined by any one of items 7 to 11 or encoding a protein as defined by any one of items 4 to 6 or;
d) optionally, a poly(A) sequence, preferably as defined by item 13;
e) optionally, a poly(C) sequence, preferably as defined by item 13;
f) optionally, a histone stem-loop, preferably as defined by any one of item 14;
g) optionally, a 3′-terminal sequence according to SEQ ID NOs: 13848-13867, 13873-13879.

Item 16:

RNA according items to any one of the preceding items comprising the following elements, preferably in 5′- to 3′-direction:
a) 5′-cap structure, preferably as defined in item 12;
b) 5′-UTR and/or 3′-UTR according to a-1, a-2, b-4, i-2, or i-3;
c) at least one coding sequence as defined by any one of items 7 to 11 wherein said coding sequence is located between said 5′-UTR and said 3′-UTR, preferably downstream of said 5′-UTR and upstream of said 3′-UTR;
d) optionally, a poly(A) sequence, preferably as defined by item 13;
e) optionally, poly(C) sequence, preferably as defined by item 13;
f) optionally, histone stem-loop, preferably as defined by any one of item 14;
g) optionally, a 3′-terminal sequence according to SEQ ID NOs: 13848-13867, 13873-13879.

Item 17:

RNA according to any one of the preceding items wherein
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2287-3566, 14056-15207 (encoding GPC) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 6107-7546, 15208-16743 (encoding GPCmut1) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 23109-24644 (encoding GPCmut2) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 24805-26340 (encoding GPCmut3) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 26501-28036 (encoding GPCmut4) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 28197-29732 (encoding GPCmut5) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 29893-31428 (encoding GPCmut6) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 31589-33124 (encoding GPCmut7) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 33285-34820 (encoding GPCmut8) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 34981-36516 (encoding GPCmut9) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 36677-38212 (encoding GPCmut10) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 38373-39908 (encoding GPCmut11) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 40069-41604 (encoding GPCmut12) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 41765-43300 (encoding GPCmut13) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 9806-11165, 16744-17967 (encoding NP) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 18104-19327, 19464-20687, 20825-22048 (encoding SP-NP) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 12804-13803, 22049-22948 (encoding Z) or a fragment or variant of any of these sequences.

Item 18:

A composition comprising at least one RNA as defined in any one of items 1 to 17, wherein the composition optionally comprises at least one pharmaceutically acceptable carrier.

Item 19:

Composition according to item 18, wherein the composition comprises more than one or a plurality, preferably 2, 3, 4, 5, 6, 7, 8, 9, 10, 11, 12, 13, 14, 15 of different RNAs each defined in any one of items 1 to 18.

Item 20:

Composition according to item 19, wherein each of the different RNAs encodes a different antigenic peptide or protein derived from the same LASV, or wherein each of the RNAs encodes a different antigenic peptide or protein derived from different proteins of the same LASV, or wherein each of the RNAs encodes a different antigenic peptide or protein derived from different proteins of different LASV.

Item 21:

Composition according to items 19 or 20, wherein the composition comprises at least one RNA encoding at least one antigenic peptide or protein derived from GPC or prefusion-stabilized GPC or a variant or fragment thereof and at least one additional RNA encoding at least one antigenic peptide or protein derived from NP (or SP-NP) or a variant or fragment thereof, and/or at least one additional RNA encoding at least one antigenic peptide or protein derived from Z or a variant or fragment thereof.

Item 22:

Composition according to items 19 to 21, wherein the composition comprises at least one RNA encoding at least one antigenic peptide or protein derived from GPC or prefusion-stabilized GPC or a variant or fragment thereof and at least one additional RNA encoding at least one antigenic peptide or protein derived from NP (or SP-NP) or a variant or fragment thereof.

Item 23:

Composition according to items 19 to 22, wherein said antigenic peptides or proteins are derived from the same LASV or from different LASV or combinations thereof.

Item 24:

Composition according to item 23, wherein the different LASV belong to different LASV clades or different LASV lineages, preferably to the LASV clades I, II, III, IV, V, and VI or to the LASV lineages I, II, III, IV, V, and VI.

Item 25:

Composition according to item 18 to 24, wherein at least one RNA is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

Item 26:

Composition according to item 25, wherein the at least one RNA is complexed or associated with one or more lipids, thereby forming liposomes, lipid nanoparticles, lipoplexes, and/or nanoliposomes.

Item 27:

Composition according to item 26, wherein the at least one RNA is complexed with one or more lipids thereby forming lipid nanoparticles (LNP).

Item 28:

Composition according to item 26 or 27, wherein the LNP comprises a cationic lipid with the formula III:

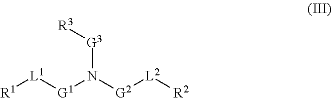

or a pharmaceutically acceptable salt, tautomer, prodrug or stereoisomer thereof, wherein:
L¹ or L² is each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}— or —NR^{a}C(═O)O—, preferably L¹ or L² is —O(C═O)—or
—(C═O)O—;
G¹ and G² are each independently unsubstituted C₁-C₁₂ alkylene or C₁-C₁₂ alkenylene;
G³ is C₁-C₂₄ alkylene, alkenylene, C₃-C₈ cycloalkylene, or C₃-C₈ cycloalkenylene;
R^{a} is H or C₁-C₁₂ alkyl;
R¹ and R² are each independently C6⁻C24 alkyl or C6⁻C24 alkenyl;
R³ is H, OR⁵, CN, —C(═O)OR⁴, —OC(═O)R⁴ or —NR⁵C(═O)R⁴;
R⁴ is 1-C₁₂ alkyl;
R⁵ is H or 1-C₆ alkyl; and
x is 0, 1 or 2;

Item 29:

Composition according to item 28, wherein the cationic lipid is a compound of formula III, and wherein:
L¹ and L² are each independently —O(C═O)— or (C═O)—O—;
G³ is C₁-C₂₄ alkylene or C₁-C₂₄ alkenylene; and
R³ is H or OR⁵.

Item 30:

Composition according to any one of items 28 to 29, wherein the cationic lipid is a compound of formula III, and wherein:
L¹ and L² are each independently —O(C═O)— or (C═O)—O—; and
R¹ and R² each independently have one of the following structures:

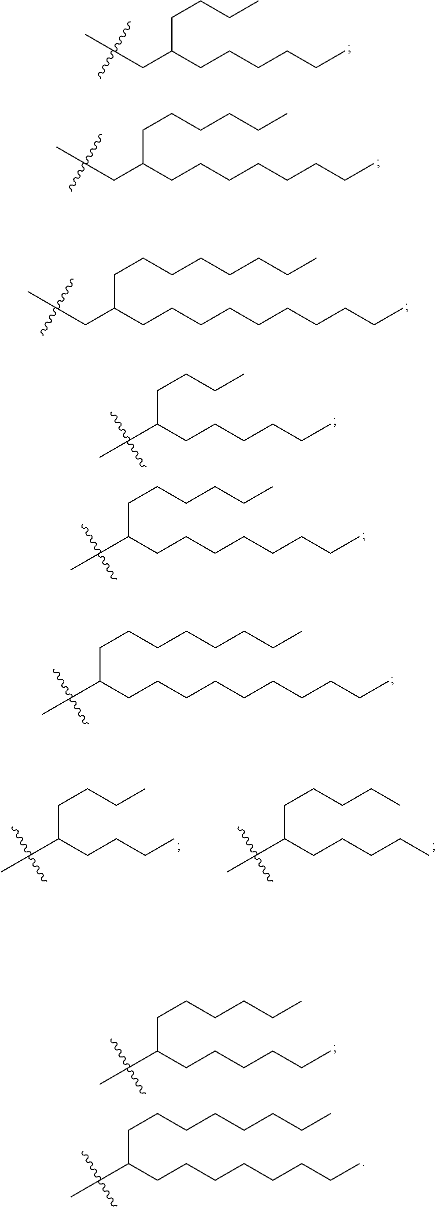

Item 31:

Composition according to any one of items 28 to 30, wherein the cationic lipid is a compound of formula III, and wherein R³ is OH.

Item 32:

Composition according to any one of items 28 to 31, wherein the cationic lipid is selected from structures III-1 to III-36:

| No. | Structure |
|---|---|
| III-1 | 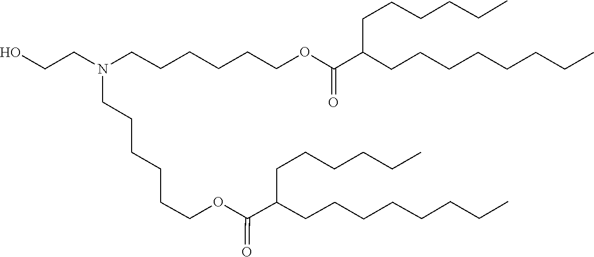 [SMILES: CCCCCCCCC(CCCCCC)C(=O)OCCCCCCN(CCO)CCCCCCOC(=O)C(CCCCCC)CCCCCCCC] |
| III-2 | 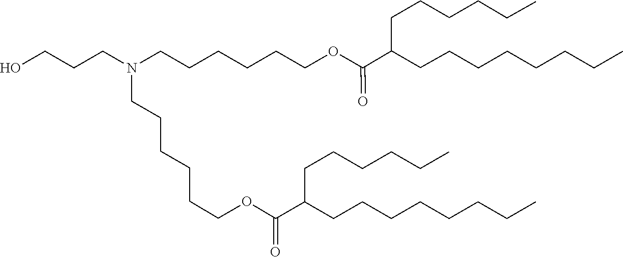 [SMILES: CCCCCCCCC(CCCCCC)C(=O)OCCCCCCN(CCCO)CCCCCCOC(=O)C(CCCCCC)CCCCCCCC] |
| III-3 | 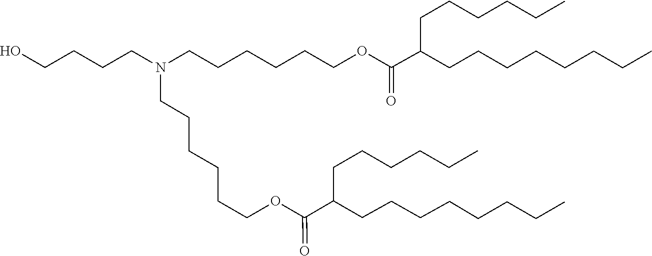 [SMILES: CCCCCCCCC(CCCCCC)C(=O)OCCCCCCN(CCCCO)CCCCCCOC(=O)C(CCCCCC)CCCCCCCC] |
| III-4 | 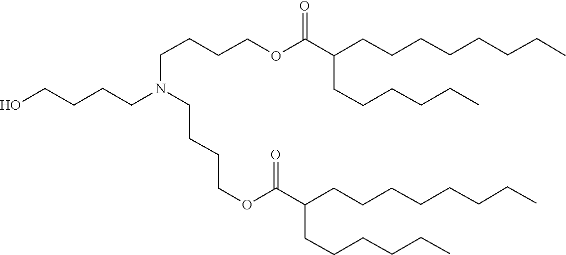 [SMILES: CCCCCCCCC(CCCCCC)C(=O)OCCCCN(CCCCO)CCCCOC(=O)C(CCCCCC)CCCCCCCC] |
| III-5 | 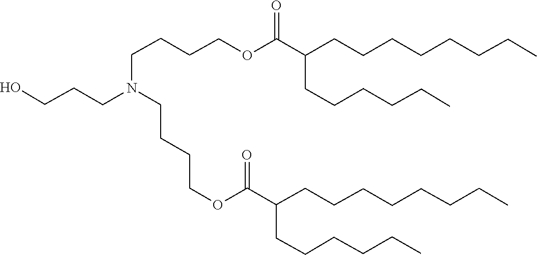 [SMILES: CCCCCCCCC(CCCCCC)C(=O)OCCCCN(CCCO)CCCCOC(=O)C(CCCCCC)CCCCCCCC] |
| III-6 | 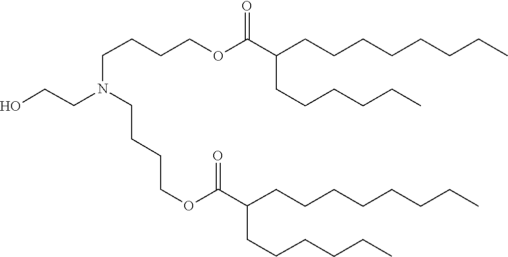 [SMILES: CCCCCCCCC(CCCCCC)C(=O)OCCCCN(CCO)CCCCOC(=O)C(CCCCCC)CCCCCCCC] |
| III-7 | 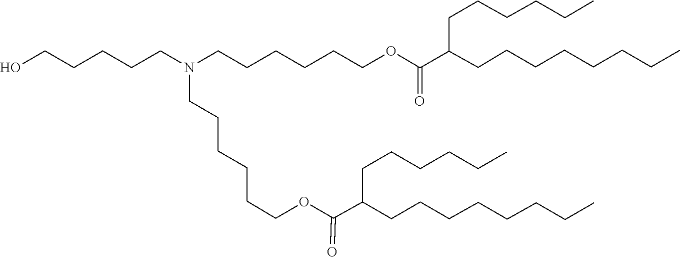 [SMILES: CCCCCCCCC(CCCCCC)C(=O)OCCCCCCN(CCCCCO)CCCCCCOC(=O)C(CCCCCC)CCCCCCCC] |
| III-8 | 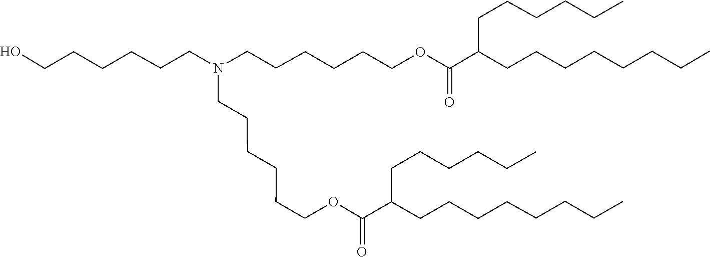 [SMILES: CCCCCCCCC(CCCCCC)C(=O)OCCCCCCN(CCCCCCO)CCCCCCOC(=O)C(CCCCCC)CCCCCCCC] |
| III-9 | 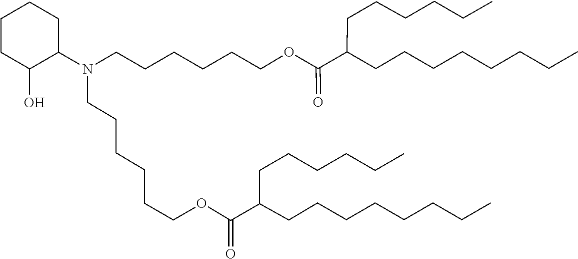 [SMILES: CCCCCCCCC(CCCCCC)C(=O)OCCCCCCN(CCCCCCOC(=O)C(CCCCCC)CCCCCCCC)C1CCCCC1O] |
| III-10 | 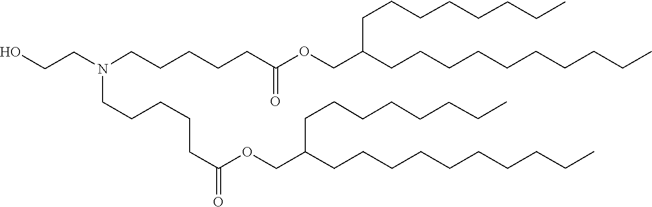 [SMILES: CCCCCCCCCCC(CCCCCCCC)COC(=O)CCCCCN(CCO)CCCCCC(=O)OCC(CCCCCCCC)CCCCCCCCCC] |
| III-11 | 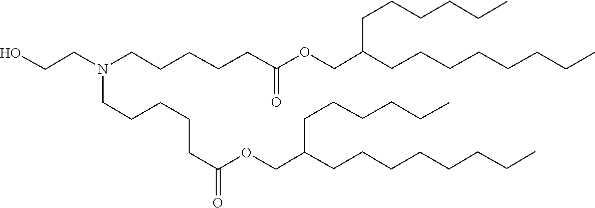 [SMILES: CCCCCCCCC(CCCCCC)COC(=O)CCCCCN(CCO)CCCCCC(=O)OCC(CCCCCC)CCCCCCCC] |
| III-12 | 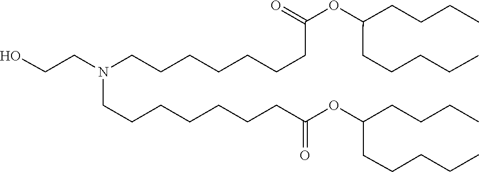 [SMILES: CCCCCC(CCCC)OC(=O)CCCCCCCN(CCO)CCCCCCCC(=O)OC(CCCC)CCCCC] |
| III-13 | 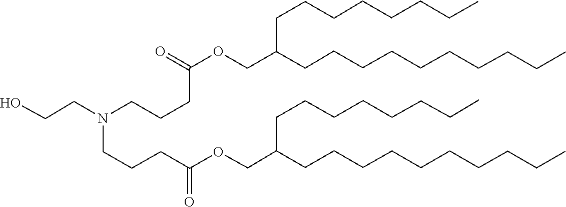 [SMILES: CCCCCCCCCCC(CCCCCCCC)COC(=O)CCCN(CCO)CCCC(=O)OCC(CCCCCCCC)CCCCCCCCCC] |
| III-14 | 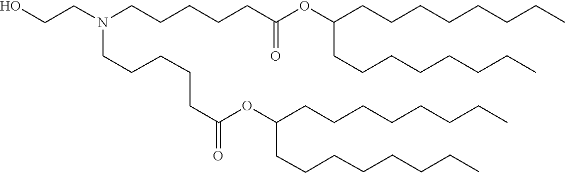 [SMILES: CCCCCCCCC(CCCCCCCC)OC(=O)CCCCCN(CCO)CCCCCC(=O)OC(CCCCCCCC)CCCCCCCC] |
| III-15 | 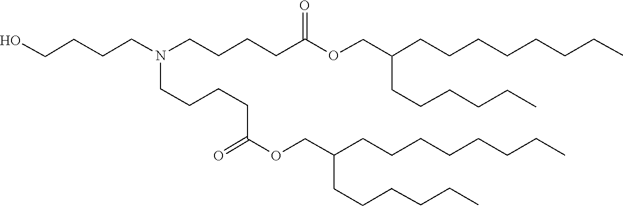 [SMILES: CCCCCCCCC(CCCCCC)COC(=O)CCCCN(CCCCO)CCCCC(=O)OCC(CCCCCC)CCCCCCCC] |
| III-16 | 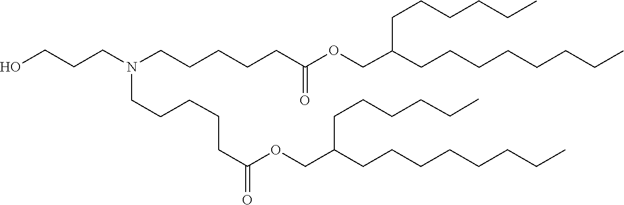 [SMILES: CCCCCCCCC(CCCCCC)COC(=O)CCCCCN(CCCO)CCCCCC(=O)OCC(CCCCCC)CCCCCCCC] |
| III-17 | 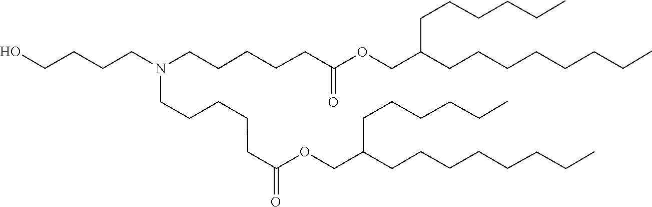 [SMILES: CCCCCCCCC(CCCCCC)COC(=O)CCCCCN(CCCCO)CCCCCC(=O)OCC(CCCCCC)CCCCCCCC] |
| III-18 | 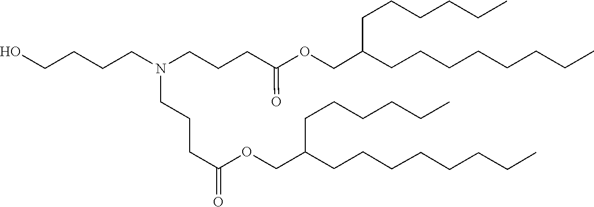 [SMILES: CCCCCCCCC(CCCCCC)COC(=O)CCCN(CCCCO)CCCC(=O)OCC(CCCCCC)CCCCCCCC] |
| III-19 | 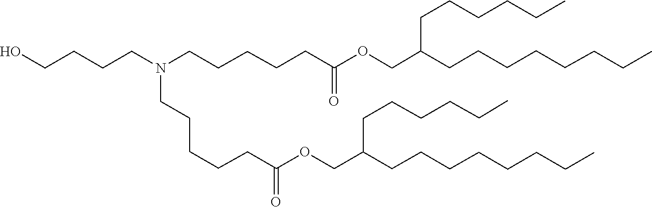 [SMILES: CCCCCCCCC(CCCCCC)COC(=O)CCCCCN(CCCCO)CCCCCC(=O)OCC(CCCCCC)CCCCCCCC] |
| III-20 | 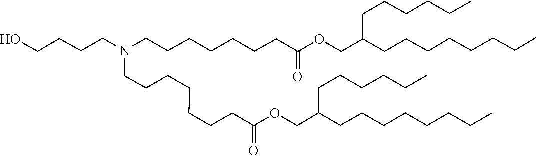 [SMILES: CCCCCCCCC(CCCCCC)COC(=O)CCCCCCCN(CCCCO)CCCCCCCC(=O)OCC(CCCCCC)CCCCCCCC] |
| III-21 | 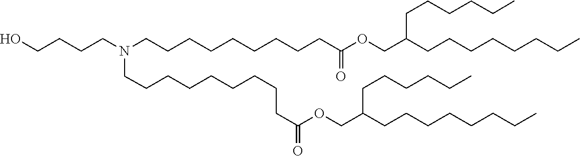 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 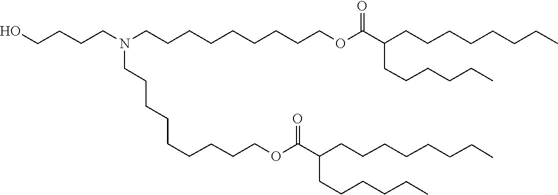 [SMILES: CCCCCCCCC(CCCCCC)C(=O)OCCCCCCCCCN(CCCCO)CCCCCCCCCOC(=O)C(CCCCCC)CCCCCCCC] |
| III-23 | 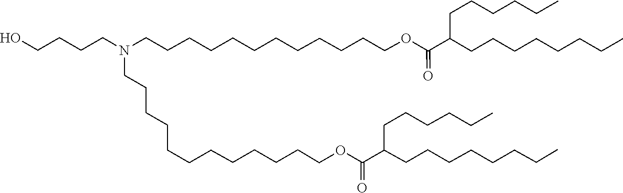 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 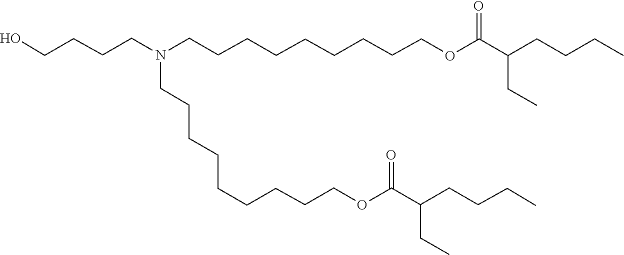 [SMILES: CCCCC(CC)C(=O)OCCCCCCCCCN(CCCCO)CCCCCCCCCOC(=O)C(CC)CCCC] |
| III-25 | 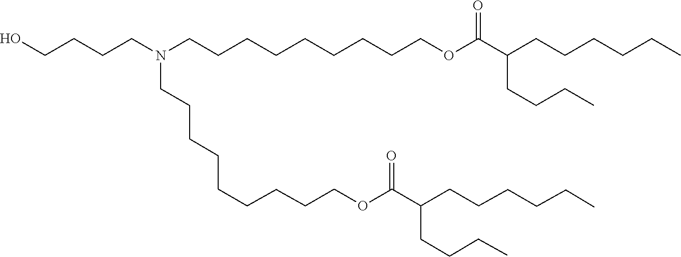 [SMILES: CCCCCCC(CCCC)C(=O)OCCCCCCCCCN(CCCCO)CCCCCCCCCOC(=O)C(CCCC)CCCCCC] |
| III-26 | 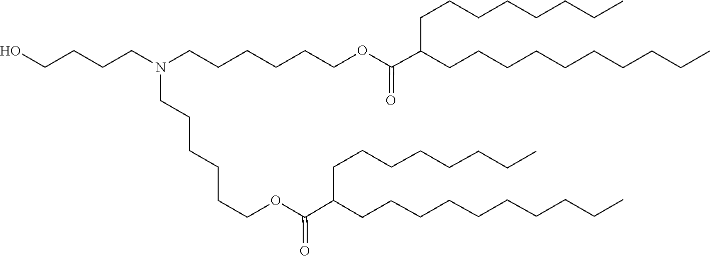 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 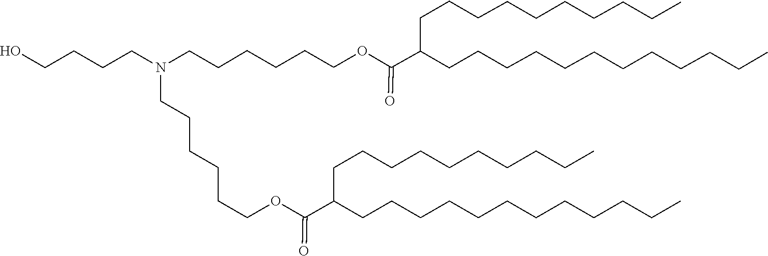 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 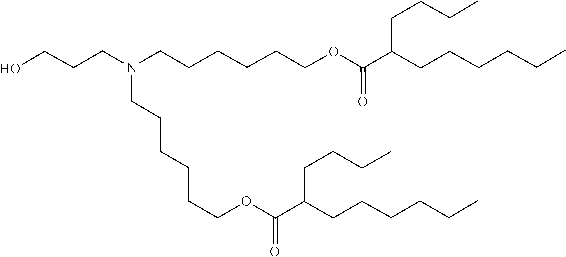 [SMILES: CCCCCCC(CCCC)C(=O)OCCCCCCN(CCCO)CCCCCCOC(=O)C(CCCC)CCCCCC] |
| III-29 | 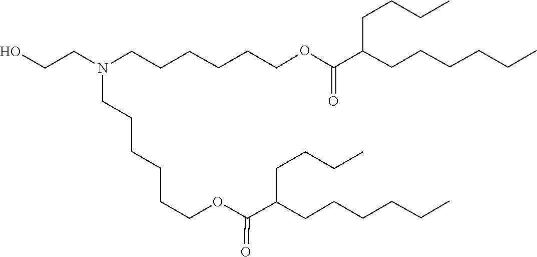 [SMILES: CCCCCCC(CCCC)C(=O)OCCCCCCN(CCO)CCCCCCOC(=O)C(CCCC)CCCCCC] |
| III-30 | 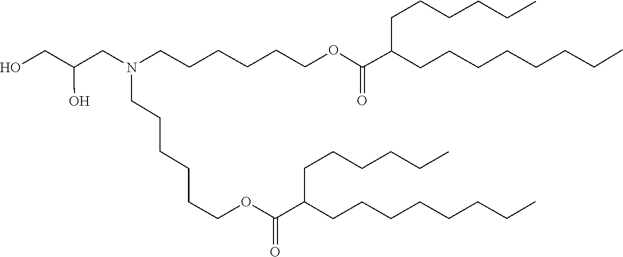 [SMILES: CCCCCCCCC(CCCCCC)C(=O)OCCCCCCN(CCCCCCOC(=O)C(CCCCCC)CCCCCCCC)CC(O)CO] |
| III-31 | 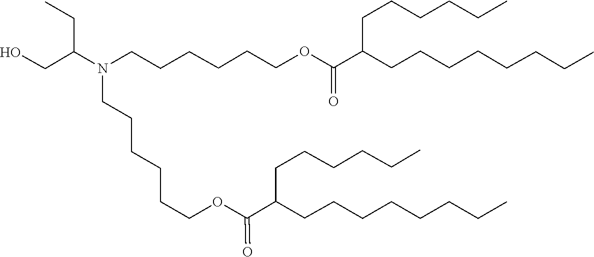 [SMILES: CCCCCCCCC(CCCCCC)C(=O)OCCCCCCN(CCCCCCOC(=O)C(CCCCCC)CCCCCCCC)C(CC)CO] |
| III-32 | 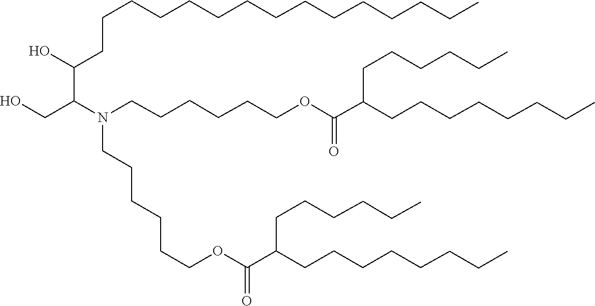 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 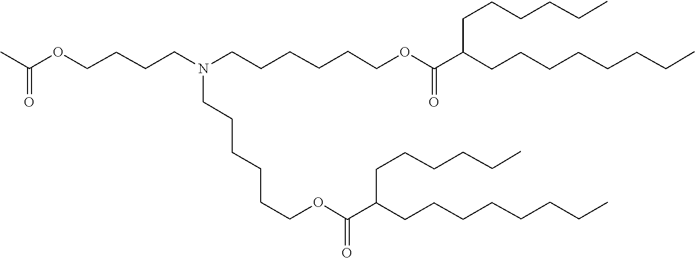 [SMILES: CCCCCCCCC(CCCCCC)C(=O)OCCCCCCN(CCCCCCOC(=O)C(CCCCCC)CCCCCCCC)CCCCOC(C)=O] |
| III-34 | 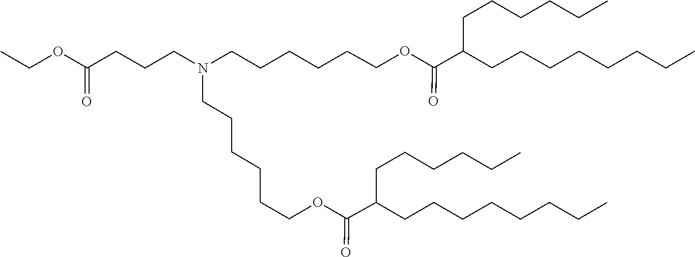 [SMILES: CCCCCCCCC(CCCCCC)C(=O)OCCCCCCN(CCCCCCOC(=O)C(CCCCCC)CCCCCCCC)CCCC(=O)OCC] |
| III-35 | 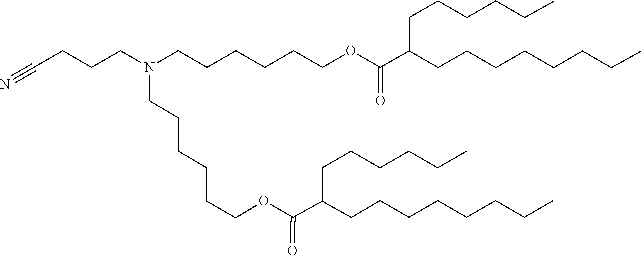 [SMILES: CCCCCCCCC(CCCCCC)C(=O)OCCCCCCN(CCCC#N)CCCCCCOC(=O)C(CCCCCC)CCCCCCCC] |
| III-36 | 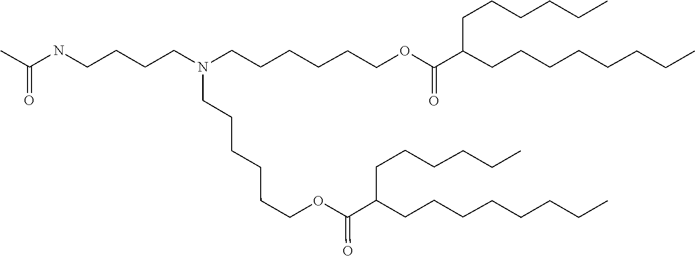 |

Item 33:

Composition according to any one of items 28 to 32, wherein the cationic lipid is

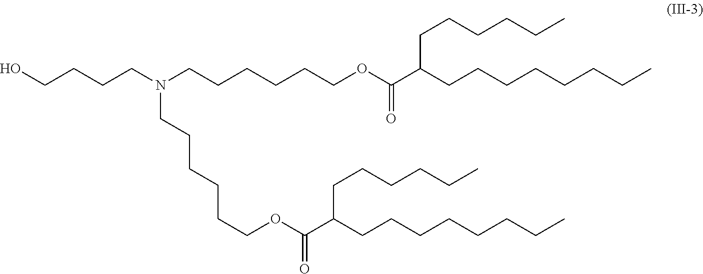 [SMILES: CCCCCCCCC(CCCCCC)C(=O)OCCCCCCN(CCCCO)CCCCCCOC(=O)C(CCCCCC)CCCCCCCC]

Item 34:

Composition according to any one of items 27 to 33, wherein the LNP comprises a PEG lipid with the formula (IV):

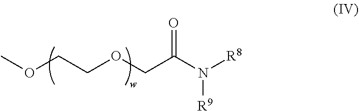

wherein R⁹ and R⁹ are each independently a straight or branched, saturated or unsaturated alkyl chain containing from 10 to 30 carbon atoms, wherein the alkyl chain is optionally interrupted by one or more ester bonds;
and w has a mean value ranging from 30 to 60.

Item 35:

Composition according to item 34, wherein in the PEG lipid R⁹ and R⁹ are saturated alkyl chains.

Item 36:

Composition according to item 34 or 35, wherein the PEG lipid is

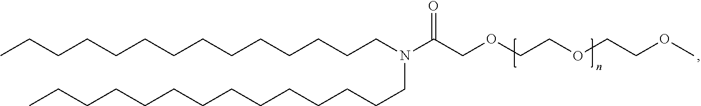

wherein n has a mean value ranging from 30 to 60, preferably wherein n has a mean value of about 45, 46, 47, 48, 49, 50, 51, 52, 53, 54, most preferably wherein n has a mean value of 49.

Item 37:

Composition according to any one of items 27 to 36, wherein the LNP comprises one or more neutral lipids and/or a steroid or steroid analogues.

Item 38:

Composition according to item 37, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE).

Item 39:

Composition according to item 37 or 38, wherein the neutral lipid is 1,2-distearoyl-sn-glycero-3-phosphocholine (DSPC), and wherein the molar ratio of the cationic lipid to DSPC is optionally in the range from about 2:1 to 8:1.

Item 40:

Composition according to item 37, wherein the steroid is cholesterol, and wherein the molar ratio of the cationic lipid to cholesterol is optionally in the range from about 2:1 to 1:1

Item 41:

Composition according to any one of items 27 to 40, wherein the LNP essentially consists of
(i) at least one cationic lipid, preferably as defined in any one of items 28 to 33;
(ii) a neutral lipid, preferably as defined in any one of items 37 to 39;
(iii) a steroid or steroid analogue, preferably as defined in item 40; and
(iv) a PEG-lipid, e.g. PEG-DMG or PEG-cDMA, preferably as defined in any one of items 34 to 36,

wherein (i) to (iv) are in a molar ratio of about 20-60% cationic lipid, 5-25% neutral lipid, 25-55% sterol, and 0.5-15% PEG-lipid.

Item 42:

A vaccine comprising at least one RNA as defined in any one of items 1 to 17, or the composition as defined in any one of items 18 to 41.

Item 43:

Vaccine according to item 42, wherein the at least one RNA as defined in any one of items 1 to 17, or the composition as defined in any one of items 18 to 41 elicits an adaptive immune response.

Item 44:

A Kit or kit of parts comprising at least one RNA as defined in any one of items 1 to 17, the composition as defined in any one of items 18 to 41, and/or the vaccine as defined in any one of items 42 or 43, optionally comprising a liquid vehicle for solubilising, and optionally technical instructions providing information on administration and dosage of the components.

Item 45:

Kit or kit of parts according to item 44 comprising at least the following components
a) at least one RNA as defined in any one of items 1 to 17 encoding at least one antigenic peptide or protein derived from a LASV, preferably derived from GPC or prefusion-stabilized GPC, wherein said RNA is preferably complexed with one or more lipids thereby forming lipid nanoparticles (LNP); and
b) at least one, two or more further RNA species as defined in any one of items 1 to 17 each encoding an antigenic peptide or protein derived from a LASV, preferably derived from NP or Z, wherein each of said further RNA species are preferably complexed with one or more lipids thereby forming lipid nanoparticles (LNP),

wherein components a) and b) are provided as separate entities or as a single entity.

Item 46:

Kit or kit of parts according to item 45 or 46 further comprising Ringer lactate solution.

Item 47:

RNA as defined in any one of items 1 to 17, the composition as defined in any one of items 18 to 41, the vaccine as defined in any one of items 42 or 43, or the kit or kit of parts as defined in item 44 to 46 for use as a medicament.

Item 48:

RNA as defined in any one of items 1 to 17, the composition as defined in any one of items 18 to 41, the vaccine as defined in any one of items 42 or 43, or the kit or kit of parts as defined in item 44 to 46 for use in the treatment or prophylaxis of an infection with a virus, preferably with LASV, or a disorder related to such an infection.

Item 49:

A method of treating or preventing an infection with a LASV, or a disorder related to such an infection, wherein the method comprises applying or administering to a subject in need thereof the at least one RNA as defined in any one of items 1 to 17, the composition as defined in any one of items 18 to 41, the vaccine as defined in any one of items 42 or 43, or the kit or kit of parts as defined in item 44 to 46.

Brief Description of Tables and Lists

List 1: LASV clade I or LASV lineage I members

List 2: LASV clade II or LASV lineage II members

List 3: LASV clade III or LASV lineage III members

List 4: LASV clade IV or LASV lineage IV members

List 5: LASV clade V or LASV lineage V members

List 6: LASV clade VI or LASV lineage VI members

Table 1: Human codon usage table with frequencies indicated for each amino acid

Table 2: Preferred LASV Glycoprotein (GPC) constructs

Table 3.1: Preferred LASV prefusion-stabilized (GPCmut1) constructs

Table 3.2: Preferred LASV prefusion-stabilized (GPCmut2) constructs

Table 3.3: Preferred LASV prefusion-stabilized (GPCmut3) constructs

Table 3.4: Preferred LASV prefusion-stabilized (GPCmut4) constructs

Table 3.5: Preferred LASV prefusion-stabilized (GPCmut5) constructs

Table 3.6: Preferred LASV prefusion-stabilized (GPCmut6) constructs

Table 3.7: Preferred LASV prefusion-stabilized (GPCmut7) constructs

Table 3.8: Preferred LASV prefusion-stabilized (GPCmut8) constructs

Table 3.9: Preferred LASV prefusion-stabilized (GPCmut9) constructs

Table 3.10: Preferred LASV prefusion-stabilized (GPCmut10) constructs

Table 3.11: Preferred LASV prefusion-stabilized (GPCmut11) constructs

Table 3.12: Preferred LASV prefusion-stabilized (GPCmut12) constructs

Table 3.13: Preferred LASV prefusion-stabilized (GPCmut13) constructs

Table 4: Preferred LASV Nucleoprotein (NP) constructs

Table 4.1: Preferred LASV Nucleoprotein (SP-HsPLAT_NP) constructs

Table 4.2: Preferred LASV Nucleoprotein (SP-HsALB_NP) constructs

Table 4.3: Preferred LASV Nucleoprotein (SP-IgE_NP) constructs

Table 5: Preferred LASV Zinc-binding matrix protein (Z) constructs

Table 6: Representative lipid compounds derived from formula (III)

Table 7: RNA constructs used in the Examples section

Table 8: Animal groups and vaccination schedule of Example 2 (see Examples section)

Table 9: Animal groups and vaccination schedule of Example 3 (see Examples section)

Table 10: Animal groups and vaccination schedule of Example 4 (see Examples section)

Table 11: Animal groups and vaccination schedule of Example 5 (see Examples section)

Table 12: Tested RNA constructs of Example 6 (see Examples section)

Table 13: mRNA designs for Western blot analysis (see Examples section)

Table 14: Animal groups and vaccination schedule of Example 7 (see Examples section)

Table 15: Animal groups and vaccination schedule of Example 8 (see Examples section)

Table 16: Animal groups and vaccination schedule of Example 9 (see Examples section)

### BRIEF DESCRIPTION OF THE DRAWINGS

FIG. 1: shows that LNP formulated RNA encoding LASV GPC induced the formation of specific binding antibodies in mice. A: IgG1 titers on day 21; B: IgG1 titers on day 35; C: IgG2a titers on day 21; D: IgG2a titers on day 35. Vaccination schedule and animal groups see Table 8. Further details are provided in Example 2.

FIG. 2: shows that LNP-formulated RNA encoding LASV GPC induced the T-cell responses. Vaccination schedule and animal groups see Table 8. Further details are provided in Example 2.

FIG. 3: shows that LNP-formulated RNA encoding LASV GPC with different mRNA design induced the formation of specific binding antibodies in mice. The experiment was performed as described in Example 4. Further construct details are provided in Table 10.

FIG. 4: shows that LNP-formulated RNA encoding LASV GPC with different mRNA designs induced T-cell responses using an intracellular cytokine staining assay. A:% of CD8/INFgamma/TNF positive T cells. B: % of CD4/INFgamma/TNF positive T cells. The experiment was performed as described in Example 4. Further construct details are provided in Table 10.

FIG. 5: shows that LNP-formulated RNA encoding an prefusion-stabilized GPC construct induced the formation of specific binding antibodies in mice. The experiment was performed as described in Example 5. Further details are provided in Table 11.

FIG. 6: shows that LNP-formulated RNA encoding an optimized LASV GPC construct induced T-cell responses using an intracellular cytokine staining assay. A:% of CD8/INFgamma/TNF positive T cells. B: % of CD4/INFgamma/TNF positive T cells. The experiment was performed as described in Example 5. Further details are provided in Table 11.

FIG. 7: shows that optimized GPC constructs encoded by optimized mRNA designs express GPC proteins. The experiment was performed as described in Example 6. Further details are provided in Table 12.

FIG. 8: shows that optimized GPC constructs encoded by optimized mRNA designs express GPC proteins. The experiment was performed as described in Example 6. Further details are provided in Table 12.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1

### Preparation of DNA and RNA Constructs and Compositions for In Vitro and In Vivo Experiments

The present Example provides methods of obtaining the RNA of the invention as well as methods of generating a composition or a vaccine of the invention.

1.1. Preparation of DNA and RNA constructs:

DNA sequences encoding different LASV antigenic proteins were prepared and used for subsequent RNA in vitro transcription. Said DNA sequences were prepared by modifying the wild type cds sequences by introducing an optimized cds (opt1, opt2, opt4, opt6, opt11). Sequences were introduced into a plasmid vector to comprise (i) advantageous 3′-UTR sequences derived from PSMB3, ALB7, alpha-globin (“muag”), or CASP1 and (ii) advantageous 5′-UTR sequences selected from HSD17B4, RPL32, NDUFA4, additionally comprising (iii) a stretch of adenosines, and optionally a histone-stem-loop structure and/or a stretch of 30 cytosines (Table 7).

Obtained plasmid DNA was transformed and propagated in bacteria using common protocols and plasmid DNA was extracted, purified, and used for subsequent RNA in vitro transcription (see section 1.2.).

Alternatively, DNA plasmids prepared according to paragraph 1 are used as DNA template for PCR-based amplification. The generated PCR products are purified and used for subsequent RNA in vitro transcription (see section 1.3.).

1.2. RNA In Vitro Transcription from Plasmid DNA Templates:

DNA plasmids prepared according to paragraph 1.1 were enzymatically linearized using a restriction enzyme (e.g. EcoRI, sapI or HindIII) and used for DNA dependent RNA in vitro transcription using T7 RNA polymerase in the presence of a nucleotide mixture (ATP/GTP/CTP/UTP) and cap analog (e.g., m7GpppG, m7G(5′)ppp(5′)(2′OMeA)pG or 3′-O-Me-m7G(5′)ppp(5′)G)) under suitable buffer conditions. The obtained RNA constructs were purified using RP-HPLC (PureMessenger®, CureVac AG, Tübingen, Germany; WO2008/077592) and used for in vitro and in vivo experiments. RNA for clinical development is produced under current good manufacturing practice e.g. according to WO2016/180430, implementing various quality control steps on DNA and RNA level. The generated RNA sequences/constructs are provided in Table 7 with the encoded antigenic protein and the respective UTR elements indicated therein. In addition to the information provided in Table 7, further information relating to specific RNA SEQ-ID NOs may be derived from the information provided under <223> identifier provided in the ST.25 sequence listing.

Alternatively, enzymatically linearized DNA is used for DNA dependent RNA in vitro transcription using an RNA polymerase in the presence of a modified nucleotide mixture (ATP, GTP, CTP, N(1)-methylpseudouridine (m1ψ), pseudouridine (ψ) or 5-methoxyuridine) and cap analog (m7GpppG, m7G(5′)ppp(5′)(2′OMeA)pG or m7G(5′)ppp(5′)(2′OMeG)pG) under suitable buffer conditions. The obtained m1ψ-modified, ψ-modified or 5-methoxyuridine modified RNA is purified e.g. as explained above and used for further experiments.

Some RNA constructs are in vitro transcribed in the absence of a cap analog. The cap-structure (cap0 or cap1) is added enzymatically using capping enzymes as commonly known in the art. In short, in vitro transcribed RNA is capped using a capping kit to obtain cap0-RNA. Cap0-RNA may be additionally modified using Cap specific 2′-O-methyltransferase to obtain cap1-RNA. CapO-RNA or Cap1-RNA is purified e.g. as explained above and used for further experiments.

1.3. RNA In Vitro Transcription from PCR Amplified DNA Templates:

Purified PCR amplified DNA templates prepared according to paragraph 1.1 are transcribed in vitro using DNA dependent T7 RNA polymerase in the presence of a nucleotide mixture (ATP/GTP/CTP/UTP) and cap analog (m7GpppG, m7G(5′)ppp(5′)(2′OMeA)pG or 3′-O-Me-m7G(5′)ppp(5′)G)) under suitable buffer conditions. Alternatively, PCR amplified DNA is transcribed in vitro using DNA dependent T7 RNA polymerase in the presence of a modified nucleotide mixture (ATP, GTP, CTP, N(1)-methylpseudouridine (m1ψ), pseudouridine (ψ) or 5-methoxyuridine) and cap analog (m7GpppG, m7G(5′)ppp(5′)(2′OMeA)pG or 3′-O-Me-m7G(5′)ppp(5′)G)) under suitable buffer conditions. Some RNA constructs are in vitro transcribed in the absence of a cap analog and the cap-structure (cap0 or cap1) is added enzymatically using capping enzymes as commonly known in the art. The obtained RNA is purified e.g. as explained above and used for further experiments.

TABLE 7
RNA constructs used in the present examples

| | | | | | 5′-UTR/ | SEQ ID | SEQ ID |
|---|---|---|---|---|---|---|---|
| | Virus | Virus | LASV | CDS | 3′-UTR; | NO: | NO: |
| RNA ID | Strain | Clade | Antigen | opt. | UTR Design | RNA | Protein |
| R6023/R6630 | Josiah | IV | GPC | opt1 | RPL32/ALB7; i-2 | 3087 | 1 |
| R6024 | Josiah | IV | NP | opt1 | RPL32/ALB7; i-2 | 10656 | 7547 |
| R6025 | Josiah | IV | Z | opt1 | RPL32/ALB7; i-2 | 13429 | 11166 |
| R6026/R6631 | LP | I | GPC | opt1 | RPL32/ALB7; i-2 | 3088 | 2 |
| R6027/R6632 | 803213 | II | GPC | opt1 | RPL32/ALB7; i-2 | 3089 | 3 |
| R6028/R6633 | GA391 | III | GPC | opt1 | RPL32/ALB7; i-2 | 3090 | 4 |
| R6717 | Josiah | IV | GPC | opt1 | —/muag; i-3 | 3343 | 1 |
| R7278 | Josiah | IV | GPC | opt1 | HSD17B4/PSMB3; a-1 | 2319 | 1 |
| R7279 | Josiah | IV | GPC | opt1 | HSD17B4/CASP1; b-4 | 2831 | 1 |
| R7280 | Josiah | IV | GPC | opt1 | Ndufa4/PSMB3; a-2 | 2575 | 1 |
| R7286 | Josiah | IV | GPC | opt2 | —/muag; i-3 | 3375 | 1 |
| R7287 | Josiah | IV | GPC | opt4 | —/muag; i-3 | 3439 | 1 |
| R7288 | Josiah | IV | GPC | opt6 | —/muag; i-3 | 3503 | 1 |
| R7289 | Josiah | IV | GPC | opt11 | —/muag; i-3 | 3535 | 1 |
| R7390 | Josiah | IV | GPCmut1 | opt1 | —/muag; i-3 | 7291 | 3567 |
| R8477 | Josiah | IV | GPC | opt1 | —/muag; i-3 | 14055 | 1 |
| R8471 | Josiah | IV | GPC | opt1 | —/muag; i-3 | 14920 | 1 |
| R8472 | Josiah | IV | GPCmut1 | opt1 | —/muag; i-3 | 16360 | 3567 |
| R8513 | Josiah | IV | GPCmut2 | opt1 | —/muag; i-3 | 24261 | 22949 |
| R8516 | Josiah | IV | GPCmut3 | opt1 | —/muag; i-3 | 25957 | 24645 |
| R8473 | Josiah | IV | GPC | opt1 | —/muag; i-3 | 14920 | 1 |
| R8474 | Josiah | IV | GPCmut1 | opt1 | —/muag; i-3 | 16360 | 3567 |
| R8514 | Josiah | IV | GPCmut2 | opt1 | —/muag; i-3 | 24261 | 22949 |
| R8517 | Josiah | IV | GPCmut3 | opt1 | —/muag; i-3 | 25957 | 24645 |
| R8475 | Josiah | IV | GPC | opt1 | HSD17B4/PSMB3; a-1 | 14152 | 1 |
| R8476 | Josiah | IV | GPCmut1 | opt1 | HSD17B4/PSMB3; a-1 | 15336 | 3567 |
| R8515 | Josiah | IV | GPCmut2 | opt1 | HSD17B4/PSMB3; a-1 | 23237 | 22949 |
| R8518 | Josiah | IV | GPCmut3 | opt1 | HSD17B4/PSMB3; a-1 | 24933 | 24645 |
| R8479 | Josiah | IV | GPCmut13 | opt1 | —/muag; i-3 | 41893 | 41605 |

1.4. Preparation of an LNP Formulated RNA Composition:

Lipid nanoparticles (LNP), cationic lipids, and polymer conjugated lipids (PEG-lipid) were prepared and tested essentially according to the general procedures described in WO2015/199952, WO2017/004143 and WO2017/075531, the full disclosures of which are incorporated herein by reference. LNP formulated RNA was prepared using an ionizable amino lipid (cationic lipid), phospholipid, cholesterol and a PEGylated lipid. Briefly, cationic lipid compound of formula 111-3, DSPC, cholesterol, and PEG-lipid of formula IVa were solubilized in ethanol at a molar ratio (%) of approximately 50:10:38.5:1.5 or 47.4:10:40.9:1.7. LNPs comprising cationic lipid compound of formula III-3 and PEG-lipid compound of formula IVa were prepared at a ratio of RNA to total Lipid of 0.03-0.04 w/w. The RNA was diluted to 0.05 mg/mL to 0.2 mg/mL in 10 mM to 50 mM citrate buffer, pH4. Syringe pumps were used to mix the ethanolic lipid solution with the RNA aqueous solution at a ratio of about 1:5 to 1:3 (vol/vol) with total flow rates above 15 ml/min. The ethanol was then removed and the external buffer replaced with a PBS buffer comprising Sucrose by dialysis. Finally, the lipid nanoparticles were filtered through a 0.2 um pore sterile filter and the LNP-formulated RNA composition was adjusted to about 1 mg/ml total RNA. Lipid nanoparticle particle diameter size was 60-90 nm as determined by quasi-elastic light scattering using a Malvern Zetasizer Nano (Malvern, UK). For other cationic lipid compounds mentioned in the present specification, the formulation process is essentially similar. The obtained LNP-formulated RNA composition (1 mg/ml total RNA) was diluted to the desired target concentration using Saline before in vivo application.

### Example 2

### Vaccination of Mice with LNP-Formulated mRNA Encoding LASV GPC

The present Example shows that LNP-formulated mRNA encoding LASV GPC induces strong humoral and cellular immune responses in mice after i.m. immunization.

RNA encoding LASV GPC was generated according to Example 1 (R6717; see Table 7) and formulated in LNPs according to Example 1.4. Female Balb/c mice were vaccinated on day 0 and day 21 intramuscularly (i.m) with doses provided in Table 8. Mice injected with saline (0.9% NaCl buffer) served as negative controls.

TABLE 8
Animal groups and vaccination schedule of Example 2

| | | | SEQ ID NO: | SEQ ID NO: | | |
|---|---|---|---|---|---|---|
| Group | Mice | Treatment | Protein | mRNA | Dose | Route |
| 1 | 6 | R6717; LNP formulated | 1 | 3343 | 10 ug | i.m. |
| 2 | 6 | R6717; LNP formulated | 1 | 3343 | 5 ug | i.m. |
| 3 | 6 | R6717; LNP formulated | 1 | 3343 | 2 ug | i.m. |
| 4 | 6 | 0.9% NaCl buffer | — | — | — | i.m. |

Determination of Binding Antibody Titers:

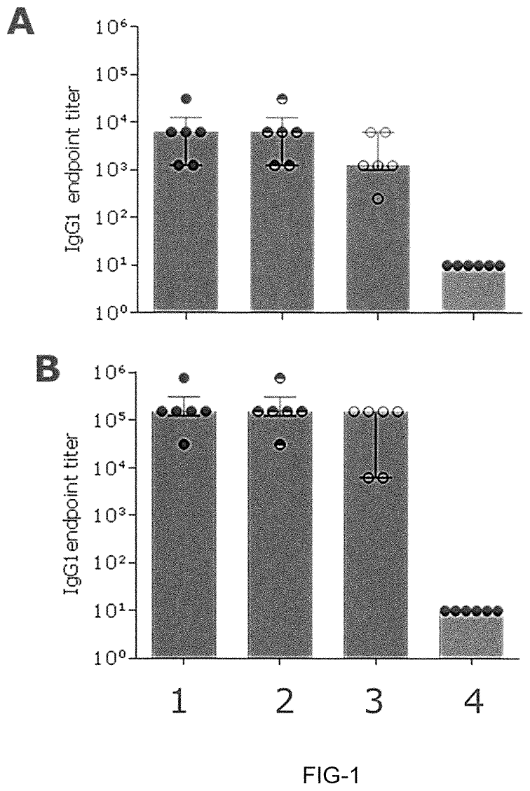

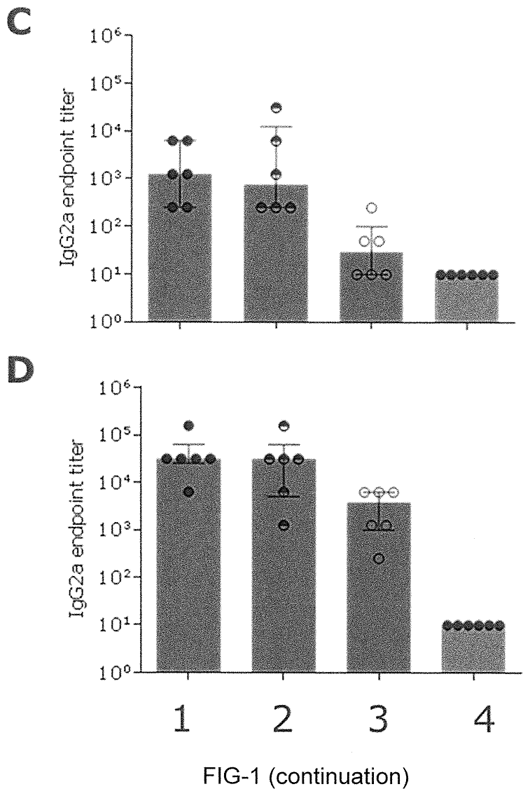

Serum samples were collected (on day 21 and day 35) and binding antibodies (IgG1 and IgG2a) were determined by ELISA using LASV GP2 protein (LASV GA391, clade I; The Native Antigen Company) for coating. Coated plates were incubated using given serum dilutions. Binding of specific antibodies to the GA391 protein was detected using biotinylated isotype specific anti-mouse antibodies in combination with streptavidin-HRP (horse radish peroxidase) with Amplex UltraRed substrate. ELISA results are shown in FIG. 1.

Intracellular Cytokine Staining (ICS):

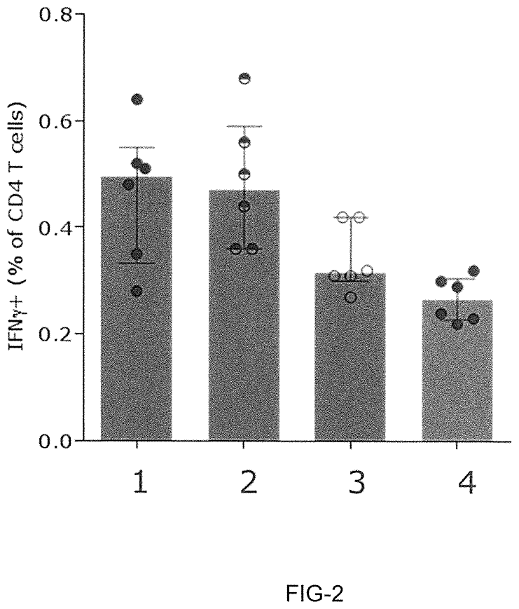

Splenocytes from vaccinated and control mice were isolated according to a standard protocol. Briefly, isolated spleens are grinded through a cell strainer and washed in PBS/1% FBS followed by red blood cell lysis. After an extensive washing step with PBS/1% FBS, splenocytes were seeded into 96-well plates (2×10⁶ cells/well). Cells were stimulated with LASV-specific CD4 and CD8 peptide mix and 2.5 ug/ml of an anti-CD28 antibody (BD Biosciences) for 6 hours at 37° C. in the presence of the mixture of GolgiPlug™/GolgiStop™ (Protein transport inhibitors containing Brefeldin A and Monensin, respectively; BD Biosciences). After stimulation cells were washed and stained for intracellular cytokines using the Cytofix/Cytoperm reagent (BD Biosciences) according to the manufacturer's instructions. The following antibodies were used for staining: CD3-FITC (1:100), CD8-PE-Cy7 (1:200), TNF-PE (1:100), IFNγ-APC (1:100) (eBioscience), CD4-BD Horizon V450 (1:200) (BD Biosciences) and incubated with Fcy-block diluted 1:100. Aqua Dye was used to distinguish live/dead cells (Invitrogen). Cells were collected using a Canto II flow cytometer (Beckton Dickinson). Flow cytometry data was analysed using FlowJo software (Tree Star, Inc.). The result of the experiment is shown in FIG. 2.

Results:

As can be seen from FIG. 1, the LNP formulated mRNA vaccine induce specific binding antibodies in mice. The data shows (i) that the mRNA construct is expressed upon injection in vivo, (ii) that the mRNA vaccine induces binding antibody responses after i.m. injection in mice, and that (iii) said binding antibody responses are induced in a dose dependent manner. Moreover, as shown in FIG. 2, LNP formulated mRNA vaccine induces CD4+ T cell responses after i.m. injection in a dose dependent manner in mice.

### Example 3

### Vaccination of Mice with Optimized LNP-Formulated mRNA Encoding LASV GPC

As demonstrated in Example 2, LNP-formulated mRNA encoding LASV GPC induces both specific humoral and specific cellular immune responses in mice. To further optimize the LASV RNA construct, e.g. to increase the expression of the RNA or the stability of the antigen, constructs comprising advantageous 3′ UTR and 5′ UTR elements (SEQ ID NO: 2319, 2831, 2575), different cds optimizations (SEQ ID NOs: 3375, 3439, 3503, 3535), or an optimized antigen (RNA construct encoding stabilized GPC; (SEQ ID NO: 7291)) are generated according to Example 1 and tested in vivo. Female Balb/c mice are vaccinated with said optimized RNA constructs on day 0 and day 21 intramuscularly (i.m) with doses provided in Table 8. Serum samples from day 21 and 35 are analyzed for binding antibody titers (as explained above) and analysis of T-cell responses is performed on splenocytes (day 35; as explained above).

TABLE 9
Animal groups and vaccination schedule of Example 3

| | | | SEQ ID NO: | | |
|---|---|---|---|---|---|
| Group | Mice | Treatment | mRNA | Dose | Route |
| 1 | 6 | R7278; LNP formulated | 2319 | 5 ug | i.m. |
| 2 | 6 | R7279; LNP formulated | 2831 | 5 ug | i.m. |
| 3 | 6 | R7280; LNP formulated | 2575 | 5 ug | i.m. |
| 4 | 6 | R7286; LNP formulated | 3375 | 5 ug | i.m. |
| 5 | 6 | R7287; LNP formulated | 3439 | 5 ug | i.m. |
| 6 | 6 | R7288; LNP formulated | 3503 | 5 ug | i.m. |
| 7 | 6 | R7289; LNP formulated | 3535 | 5 ug | i.m. |
| 8 | 6 | R7290; LNP formulated | 7291 | 5 ug | i.m. |
| 9 | 6 | 0.9% NaCl buffer | — | — | i.m. |

### Example 4

### Vaccination of Mice with Optimized LNP-Formulated mRNA Encoding LASV GPC

The present Example shows that LNP-formulated mRNA designs encoding LASV GPC induced strong humoral and cellular immune responses in mice after i.m. immunization.

RNA encoding LASV GPC was generated according to Example 1 (see Table 7) and formulated in LNPs according to Example 1.4. Female Balb/c mice were vaccinated on day 0 and 21 intramuscularly (i.m) with doses of 5 ug (further details provided in Table 10).

TABLE 10
Animal groups and vaccination schedule of Example 4

| | | | | 5′-UTR/ | |
|---|---|---|---|---|---|
| | | | | 3′-UTR; | SEQ ID NO: |
| Group | Mice | RNA ID | Treatment | UTR Design | mRNA |
| A | 5 | — | 0.9% NaCl buffer | —/— | — |
| | | | (negative control) | | |
| B | 5 | R6717 | GPC | —/muag | 3343 |
| C | 5 | R7278 | GPC | HSD17B4/PSMB3, a-1 | 2319 |
| D | 5 | R7279 | GPC | HSD17B4/CASP1, b-4 | 2831 |
| E | 5 | R7280 | GPC | Ndufa4/PSMB3, a-2 | 2575 |

Determination of Binding Antibody Titers:

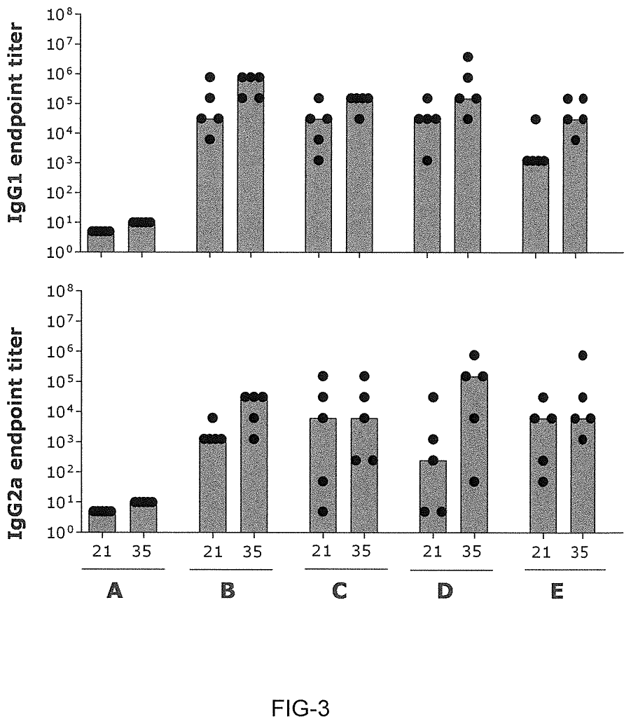

Serum samples were collected (on day 21 and day 35) and binding antibodies (IgG1 and IgG2a) were determined by ELISA using LASV GP2 protein (LASV GA391, clade I; The Native Antigen Company) for coating. Coated plates were incubated using given serum dilutions. Binding of specific antibodies to the GA391 protein was detected using biotinylated isotype specific anti-mouse antibodies in combination with streptavidin-HRP (horse radish peroxidase) with Amplex UltraRed substrate. ELISA results are shown in FIG. 3.

Intracellular Cytokine Staining (ICS):

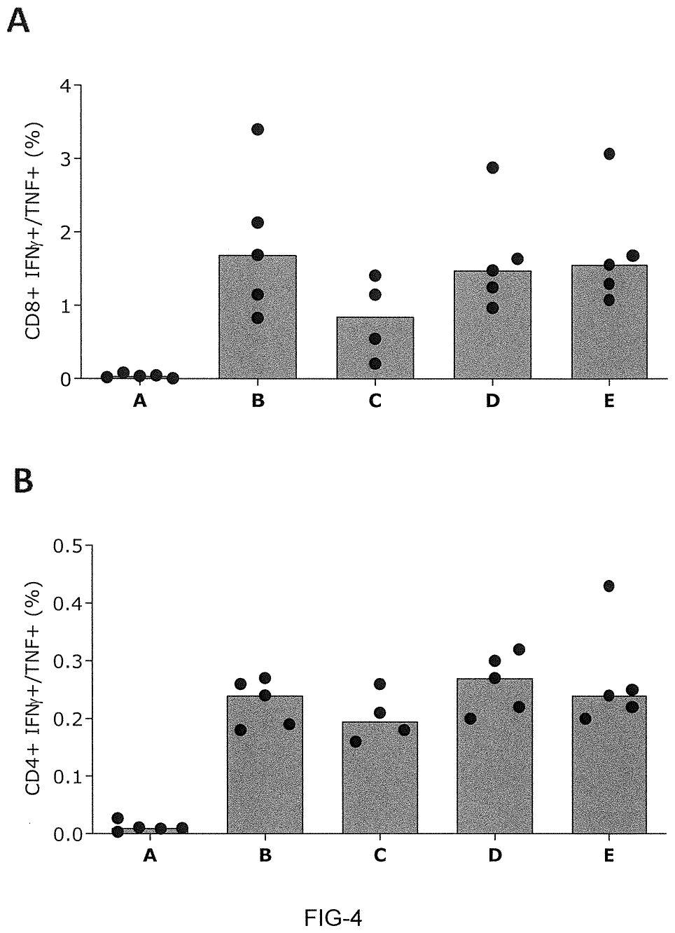

Splenocytes from vaccinated and control mice (see Table 10) were isolated according to a standard protocol. Briefly, isolated spleens were grinded through a cell strainer and washed in PBS/1%FBS followed by red blood cell lysis. After an extensive washing step with PBS/1%FBS, splenocytes were seeded into 96-well plates (2×10⁶ cells/well). Cells were stimulated with a LASV-specific CD4 and CD8 peptide mix and 2.5 ug/ml of an anti-CD28 antibody (BD Biosciences) for 6 hours at 37° C. in the presence of the mixture of GolgiPlug™/GolgiStop™ (Protein transport inhibitors containing Brefeldin A and Monensin, respectively; BD Biosciences). After stimulation cells were washed and stained for intracellular cytokines using the Cytofix/Cytoperm reagent (BD Biosciences) according to the manufacturer's instructions. The following antibodies were used for staining: CD3-FITC (1:100), CD8-PE-Cy7 (1:200), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells are collected using a Canto II flow cytometer (Beckton Dickinson). Flow cytometry data was analysed using FlowJo software (Tree Star, Inc.). The result of the experiment is shown in FIG. 4.

Results:

As can be seen from FIG. 3, the LNP formulated mRNA vaccine with different mRNA designs (5′-UTR and 3′-UTR-combinations, see Table 10) induced specific binding IgG1 and IgG2a antibodies in mice. The results show (i) that the mRNA construct was expressed upon injection in vivo and (ii) that the mRNA vaccine induced binding antibody responses after i.m. injection in mice.

Moreover, as shown in FIG. 4A, LNP formulated mRNA vaccine with different mRNA designs (5′-UTR and 3′-UTR-combinations, see Table 10) induced CD8+ T cell responses after i.m. injection in in mice. In addition, the LNP formulated mRNA vaccine induced CD4+ T cell responses (see FIG. 4B). Both T-cell responses play important roles in the adaptive immune response against viral pathogens such as LASV.

### Example 5

### Vaccination of Mice with LNP-Formulated mRNA Encoding an Optimized LASV GPC Construct

The present Example shows that LNP-formulated RNA encoding a LASV prefusion stabilized GPC induce strong humoral and cellular immune responses in mice after i.m. immunization.

Determination of Binding Antibodies:

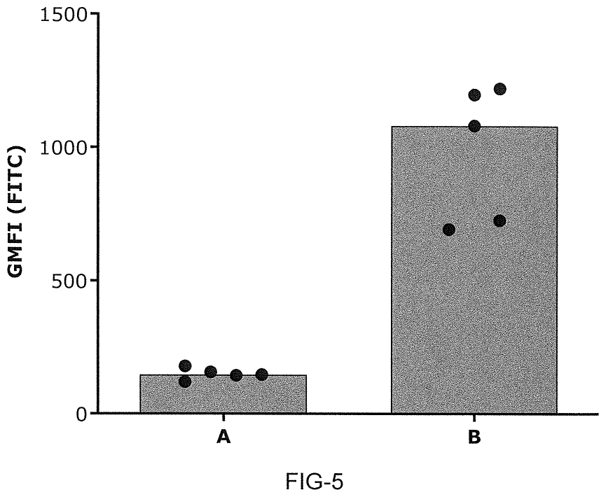

HeLa cells were transfected with 2 ug RNA prefusion-stablized GPC (GPCmut1, R7390) using Lipofectamine. 20 h post transfection cells were harvested, counted and seeded at 1×10⁵ cells/well of a 96 well U-bottom plate. Cells were incubated with serum (diluted 1:50) collected two weeks post second immunization of Balb/c mice (as previously described in Example 4) with 5 ug R7390 (RNA encoding GPCmut1, Group B of Table 11) or NaCl buffer (group A), followed by secondary anti-mouse FITC-conjugated antibody. Cells were acquired on a BD FACS Canto II and analyzed using FlowJo software. The result of the experiment is shown in FIG. 5.

Intracellular Cytokine Staining (ICS):

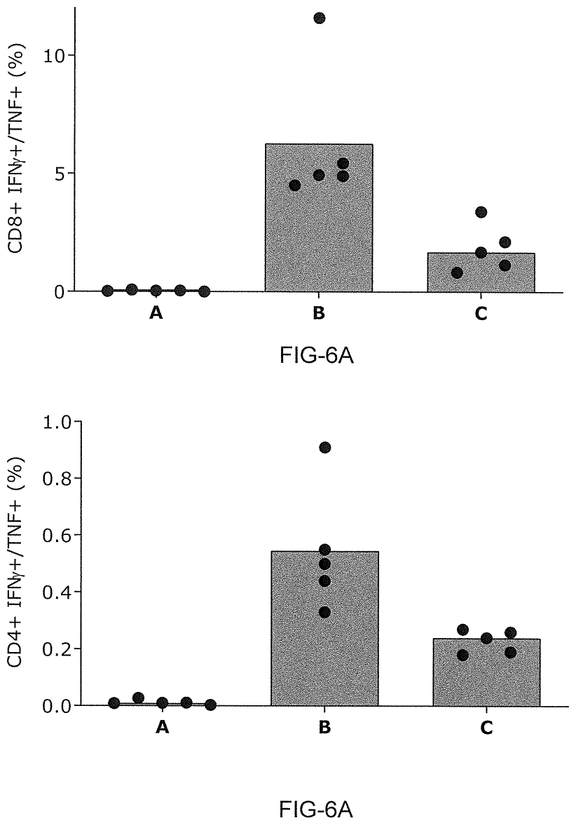

Splenocytes from vaccinated and control mice (see Table 11, Group A-C) were isolated according to a standard protocol. Briefly, isolated spleens are grinded through a cell strainer and washed in PBS/1% FBS followed by red blood cell lysis. After an extensive washing step with PBS/1% FBS, splenocytes were seeded into 96-well plates (2×10⁶ cells/well). Cells were stimulated with a LASV-specific CD4 and CD8 peptide mix and 2.5 ug/ml of an anti-CD28 antibody (BD Biosciences) for 6 hours at 37° C. in the presence of the mixture of GolgiPlug™/GolgiStop™ (Protein transport inhibitors containing Brefeldin A and Monensin, respectively; BD Biosciences). After stimulation cells were washed and stained for intracellular cytokines using the Cytofix/Cytoperm reagent (BD Biosciences) according to the manufacturer's instructions. The following antibodies were used for staining: CD3-FITC (1:100), CD8-PE-Cy7 (1:200), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells are collected using a Canto II flow cytometer (Beckton Dickinson). Flow cytometry data was analysed using FlowJo software (Tree Star, Inc.) The result of the experiment is shown in FIG. 6.

TABLE 11
Animal groups and vaccination schedule of Example 5

| | | | Construct | SEQ ID NO: | SEQ ID NO: |
|---|---|---|---|---|---|
| Group | Mice | Treatment | Design | mRNA | mRNA |
| A | 5 | 0.9% NaCl | — | — | |
| | | buffer | | | |
| | | (negative | | | |
| | | control) | | | |
| B | 5 | R7390 | GPCmut1 | 7291 | 3567 |
| C | 5 | R6717 | GPC | 3343 | 1 |

Results:

As can be seen from FIG. 5, the LNP formulated mRNA encoding a LASV prefusion-stabilized GPC construct (see Table 11, Group B) induced specific binding antibodies in mice. The result shows (i) that the prefusion-stabilized GPC construct was expressed upon injection in vivo and additionally (ii) that the mRNA construct induced binding antibody responses after i.m. injection in mice.

Additionally, as shown in FIG. 6A and FIG. 6B the LNP formulated mRNA encoding a LASV prefusion-stabilized GPC construct induced robust CD8 and CD4 positive T cell responses. In addition the optimized LASV prefusion-stabilized GPC mRNA vaccine (Group B, see FIG. 6A and B) showed highly improved T cell responses compared to the wt LASV GPC construct (Group C, see FIGS. 6A and B).

### Example 6

### In Vitro Expression Analysis of Optimized mRNA Designs Encoding LASV GPC and Prefusion-Stabilized GPC constructs

The present Example shows that optimized mRNA designs according to the invention (UTR-combination a-1, cap1, and/or poly(A)-sequence located (exactly) at the 3′ end) encoding different optimized LASV GPC constructs express GPC and prefusion-stablized GPC proteins. As a detection antibody, the mouse 37.7H monoclonal antibody was used, which is directed against the quaternary GPC-B epitope. The use of this antibody gives indication for a correct conformation of the heterotrimeric GPC protomer to potentially induce neutralizing antibodies that achieve neutralization by stabilizing LASV GPC in its prefusion conformation.

FACS Analysis:

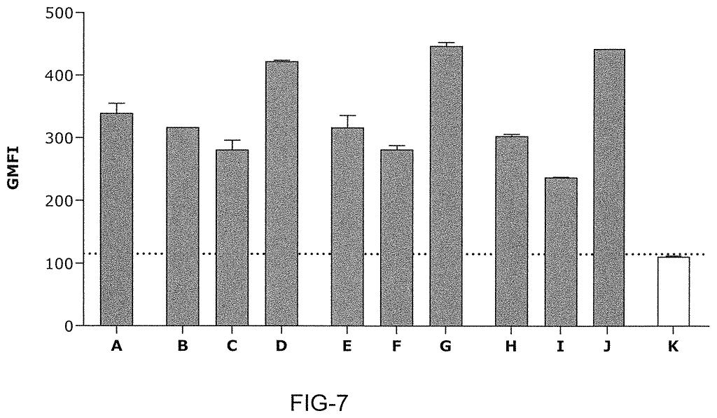

HeLa cells were transfected with 2 ug of the different mRNA designs (see Table 12) using Lipofectamine. 20 h post transfection cells were harvested, and seeded in a 96 well U-bottom plate. Cells were incubated with α-GPC [37.7H] antibody which binds to a quaternary GPC-B epitope bridging the LASV GP1 and GP2 subunits,)) encoded by different mRNA constructs (see Table 12) followed by secondary anti-mouse FITC-conjugated antibody. Cells were acquired on a BD FACS Canto II and analyzed using FlowJo software. The result of the experiment is shown in FIG. 7.

TABLE 12
Tested RNA constructs of Example 6

| | | | 5′-UTR/ | 5′-cap | | |
|---|---|---|---|---|---|---|
| | | | 3′-UTR; | structure/ | Modified | SEQ ID |
| Group | RNA ID | Construct Design | UTR Design | 3′ terminus | nucleotides | NO: mRNA |
| A | R8477 | GPC full-length | —/muag | cap1/ | — | 14055 |
| | | | | A64-N5-hSL-N5 | | |
| B | R8471 | GPC full-length | —/muag | cap1/ | — | 14920 |
| | | | | hSL-A64-N5 | | |
| C | R8472 | GPCmut1 | —/muag | cap1/ | — | 16360 |
| | | | | hSL-A64-N5 | | |
| D | R8513 | GPCmut2 | —/muag | cap1/ | — | 24261 |
| | | | | hSL-A64-N5 | | |
| E | R8473 | GPC full-length | —/muag | cap1/ | Ψ | 14920 |
| | | | | hSL-A64-N5 | | |
| F | R8474 | GPCmut1 | —/muag | cap1/ | Ψ | 16360 |
| | | | | hSL-A64-N5 | | |
| G | R8514 | GPCmut2 | —/muag | cap1/ | Ψ | 24261 |
| | | | | hSL-A64-N5 | | |
| H | R8475 | GPC full-length | HSD17B4/ | cap1/ | — | 14152 |
| | | | PSMB3; a-1 | hSL-A100 | | |
| I | R8476 | GPCmut1 | HSD17B4/ | cap1/ | — | 15336 |
| | | | PSMB3; a-1 | hSL-A100 | | |
| J | R8515 | GPCmut2 | HSD17B4/ | cap1/ | — | 23237 |
| | | | PSMB3; a-1 | hSL-A100 | | |
| K | | Negative control (WFI = | — | — | — | — |
| | | water for injection) | | | | |

Western Blot Analysis:

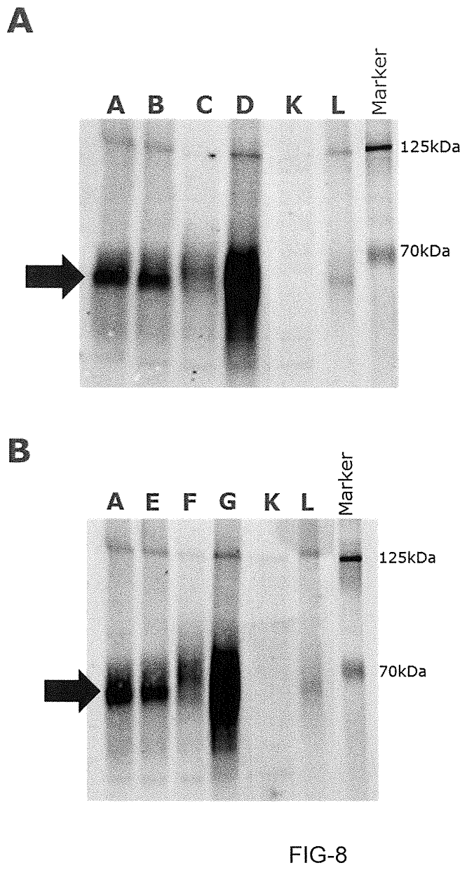

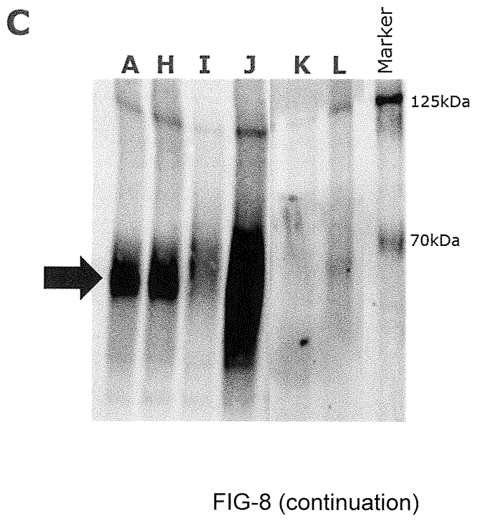

HeLa cells were transfected with 2 ug of the respective mRNA designs encoding LASV GPC (see Table 13), with a negative control (water for injection) and as a positive control (irradiated LASV Josiah strain, clade IV). 20 h post transfection cells were harvested, lysed and subjected to SDS-PAGE followed by Western blot. For the detection a-GPC [37.7H] antibody was used and a goat anti-mouse IgG IRDye® 800CW antibody (1:10000; Li-Cor) as secondary antibody. The a-GPC [37.7H] antibody binds to a quaternary GPC-B epitope bridging the LASV GP1 and GP2 subunits. The result of the experiment is shown in FIG. 8.

TABLE 13
mRNA designs for Western blot analysis

| | | | 5′-UTR/ | 5′-cap | | |
|---|---|---|---|---|---|---|
| | | | 3′-UTR; | structure/ | Modified | mRNA |
| Group | RNA ID | Construct Design | UTR Design | 3′ terminus | nucleotides | SEQ ID NO |
| A | R8471 | GPC full-length | —/muag | cap1/ | — | 14920 |
| | | | | hSL-A64-N5 | | |
| B | R8477 | GPC full-length | —/muag | cap1/ | — | 14055 |
| | | | | A64-N5-hSL-N5 | | |
| C | R8472 | GPCmut1 | —/muag | cap1/ | | 16360 |
| | | | | hSL-A64-N5 | | |
| D | R8513 | GPCmut2 | —/muag | cap1/ | | 24261 |
| | | | | hSL-A64-N5 | | |
| E | R8473 | GPC full-length | —/muag | cap1/ | Ψ | 14920 |
| | | | | hSL-A64-N5 | | |
| F | R8474 | GPCmut1 | —/muag | cap1/ | Ψ | 16360 |
| | | | | hSL-A64-N5 | | |
| G | R8514 | GPCmut2 | —/muag | cap1/ | Ψ | 24261 |
| | | | | hSL-A64-N5 | | |
| H | R8475 | GPC full-length | HSD17B4/ | cap1/ | — | 14152 |
| | | | PSMB3; a-1 | hSL-A100 | | |
| I | R8476 | GPCmut1 | HSD17B4/ | cap1/ | | 15336 |
| | | | PSMB3; a-1 | hSL-A100 | | |
| J | R8515 | GPCmut2 | HSD17B4/ | cap1/ | — | 23237 |
| | | | PSMB3; a-1 | hSL-A100 | | |
| K | | Negative control (WFI = | | | | |
| | | water for injection) | | | | |
| L | | Irradiated LASV (positive | | | | |
| | | control) | | | | |

Results:

FIG. 7 shows that all optimized mRNA constructs express a (prefusion-stabilized) GPC protein. The samples D, G and J (see Table 12, encoding prefusion-stabilizd GPC mut2) show higher levels of expressed protein.

As shown in FIG. 8, all optimized constructs (see Table 13) express a (prefusion-stabilized) GPC protein (about 76 kDa). According to the expression showed via FACS analysis, especially the Groups D, G and J, encoding the prefusion-stabilized GPC mut2 showed an increased expression.

Through the detection with the 37.7H antibody directed to the quaternary GPC-B epitope, indication for a correct conformation of the GPC trimer to potentially induce neutralizing antibodies is given.

### Example 7

### Analysis of Polyvalent LASV Vaccine Compositions in Guinea Pigs (Challenge)

The present example shows that LASV mRNA vaccines induce protective immune responses against Lassa virus infection in Hartley guinea pigs. Hartley (outbred) guinea pigs are widely used for studying arenaviral hemorrhagic fevers and for testing potential therapeutics and vaccine candidates. Binding antibodies are measured using ELISA and moreover virus neutralizing antibodies to the vaccine are analyzed. In addition this example shows of the monovalent LASV-GPC lineage IV vaccine as well as the feasibility of a 1-dose regimen.

Different mRNA designs encoding different LASV GPC constructs are prepared according to Example 1. The mRNAs are formulated with LNPs (see Example 1.4.). The different mRNA vaccine candidates (see Table 14) are applied on day 0 and 28 or only at day 0 and administered intramuscular (i.m.) with different doses of RNA. Blood samples are collected at day 0, 28, 56 and post challenge for determination of humoral immune responses.

4 weeks after the last vaccination the groups are challenged with 10⁵ PFU Josiah LASV strain (i.p. route). The groups are observed for 4 weeks after the challenge. The animals are monitored daily for survival, body weight, morbidity index, temperature and viremia. Additional the viral load in lung, spleen and kidney is measured at necropsy.

TABLE 14
Animal groups and vaccination schedule of Example 7

| | Administration | Guinea | |
|---|---|---|---|
| Group | Interval | pigs | Construct Design |
| A | 1 x | 10 | 0.9% NaCl buffer |
| | | | (negative control) |
| B | 1 x | 10 | GPC or prefusion-stabilized GPC |
| C | 2 x | 10 | GPC or prefusion-stabilized GPC |

### Example 8

### Analysis of Polyvalent LASV Vaccine Compositions in Guinea Pigs (Challenge)

The present example shows the efficacy of the mRNA LASV vaccine to provide protection against heterologous LASV strains or clades, especially that the polyvalent/tetravalent mRNA vaccine provides protection against two LASV viruses from phylogenetically most distant LASV lineages (I and IV) in guinea pigs.

mRNA designs encoding LASV GPC or prefusion-stabilized GPC are prepared according to Example 1. The mRNAs are formulated with LNPs (see Example 1.4.). The different mRNA vaccine candidates (see Table 15) are applied on day 0 and 28 and administered intramuscular (i.m.) with different doses of RNA. Blood samples are collected at day 0, 28, 56 and post challenge for determination of humoral immune responses.

4 weeks after the last vaccination the groups are challenged with 10⁵ PFU Josiah LASV strain (i.p. route). The groups are observed for 4 weeks after the challenge. The animals are monitored daily for survival, body weight, morbidity index, temperature and viremia. Additional the viral load in lung, spleen and kidney is measured at necropsy.

TABLE 15
Animal groups and vaccination schedule of Example 8

| Group | Guinea pigs | Virus Clade | Vaccine composition |
|---|---|---|---|
| A | 10 | — | 0.9% NaCl buffer |
| | | | (negative control) |
| B | 10 | IV | GPC or prefusion-stabilized GPC |
| C | 10 | I + II + | Tetravalent GPC or tetravalent |
| | | III + IV | prefusion-stabilized GPC |

### Example 9

### Analysis of Polyvalent LASV mRNA Vaccine

The present example shows a tetravalent mRNA vaccine covering Lassa clades I-IV as well as the inclusion of the nucleoprotein (NP) as an additional target of T cell responses to broaden the coverage of the vaccine.

RNA encoding different Lassa mRNA vaccine encoding GPC or a prefusion-stabilized GPC (see Table 16) was generated according to Example 1 and formulated in LNPs according to Example 1.4. Female CBA/J mice (9 mice per group) were vaccinated on day 0 and day 21 intramuscularly (i.m). Serum is collected at day 21 and 28 to test humoral immune responses. Splenocytes are collected at day 28 to test cellular immune responses via an ICS with LASV-GPC overlapping peptide libraries from lineages IV and I, II, III to analyze cross-reactivity.

TABLE 16
Animal groups and vaccination schedule of Example 9

| Group | Mice | Virus Clade | Vaccine composition |
|---|---|---|---|
| A | 9 | — | 0.9% NaCl buffer (negative control) |
| B | 9 | IV | GPC or prefusion-stabilized GPC and |
| | | | NP or SP-NP |
| C | | IV | NP or SP-NP |
| D | 9 | I + II + | Tetravalent GPC or tetravalent |
| | | II + IV | prefusion-stabilized GPC |
| E | 9 | I | GPC or prefusion-stabilized GPC |
| F | 9 | II | GPC or prefusion-stabilized GPC |
| F | 9 | III | GPC or prefusion-stabilized GPC |
| G | 9 | IV | GPC or prefusion-stabilized GPC |

### Example 9

### Safety, Reactogenicity and Immunogenicity of LASV mRNA Vaccine in Healthy Adults

To demonstrate safety, reactogenicity and immunogenicity of LASV mRNA vaccine, a phase I clinical trial is initiated.

For clinical development, RNA is used that has been produced under GMP conditions (e.g. using a procedure as described in WO2016/180430).

In this LASV mRNA vaccine phase I trial different dosages of the candidate LASV mRNA vaccine will be administered in a one or two-dose schedule to healthy adult subjects. The subjects will be enrolled sequentially into the different trial groups to receive one or two doses of LASV mRNA vaccine. The subjects in the two-dose groups will be administered a second dose 28 days later. An additional group of control subjects will receive a single dose of saline on Day 1. Safety information for solicited (days 1-7 post-vaccination) and unsolicited (days 1-28 post-vaccination) adverse events (AEs) will be collected using diary cards. Serious AEs, AEs leading to premature withdrawal from the trial or receipt of the second dose, AEof Special Interest and medically-attended AEs will be collected throughout the trial (Day 1 to Day 365 post last vaccine dose). Specified safety data will be reviewed by an internal safety review team and a DSMB on a pre-defined schedule.

1. RNA comprising at least one coding sequence encoding at least one antigenic peptide or protein derived from a Lassa virus (LASV) protein or a fragment or variant thereof, wherein said coding sequence is operably linked to a 5′-UTR derived from a HSD17B4 gene, a NDUFA4 gene, or a RPL32 gene and/or a 3′-UTR derived from a PSMB3 gene, a CASP1 gene, an ALB7 gene, or an alpha-globin gene.

2. RNA according to claim 1, wherein said coding sequence is operably linked to a 5′-UTR and/or 3′-UTR, comprising
a-1. at least one 5′-UTR derived from a 5′-UTR of a HSD17B4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-2. at least one 5′-UTR derived from a 5′-UTR of a NDUFA4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
b-4. at least one 5′-UTR derived from a 5′-UTR of a HSD17B4 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
i-2. at least one 5′-UTR derived from a 5′-UTR of a RPL32 gene, or from a corresponding RNA sequence, homolog, fragment or variant thereof and at least one 3′-UTR derived from a 3′-UTR of a ALB7 gene, or from a corresponding RNA sequence, homolog, fragment or variant thereof; or
i-3. at least one 3′-UTR derived from a 3′-UTR of a alpha-globin gene gene, or from a corresponding RNA sequence, homolog, fragment or variant thereof.

3. RNA according to claim 1 or 2, wherein
said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13804 or 13805 or a fragment or a variant thereof;
said 5′-UTR derived from a NDUFA4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13812 or 13813 or a fragment or a variant thereof;
said 5′-UTR derived from a RPL32 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13824 or 13825 or a fragment or a variant thereof;
said 3′-UTR derived from a PSMB3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13826 or 13827 or a fragment or a variant thereof;
said 3′-UTR derived from a CASP1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13828 or 13829 or a fragment or a variant thereof;
said 3′-UTR derived from a ALB7 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 13838 or 13839 or a fragment or a variant thereof;
said 3′-UTR derived from a alpha-globin gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to EQ ID NO: 13840 or 13841 or a fragment or a variant thereof.

4. RNA according to any one of the preceding claims, wherein the LASV protein is derived from glycoprotein precursor (GPC), a prefusion-stabilized GPC, nucleoprotein (NP), zinc-binding matrix protein (Z), or a variant, fragment, or combination thereof, wherein GPC, prefusion-stabilized GPC, NP, Z are preferably full-length proteins.

5. RNA according claim 4, wherein the prefusion-stabilized GPC comprises at least one of the following mutations A, preferably of A1, A2, A3 and B, and C:
A amino acid substitutions allowing a covalent link of different structural elements of GPC, preferably by introduction of two additional cysteine residues, wherein preferably
A1 the amino acid substitutions allowing a covalent link of GP1 and GP2, preferably by introduction of two additional cysteine residues, preferably at position 207 or 206 and 360 or 359, preferably by amino acid substitutions R2070 or R2060 and G3600 or G359C, or
A2 the amino acid substitutions allowing a covalent link of GP1 to HR1 of GP2, preferably by introduction of two additional cysteine residues, preferably at position 81 or 80 and 319 or 318, preferably by amino acid substitutions T81 C or T800 and N3190 or N3180, or
A3 the amino acid substitutions allowing a covalent link of T-loop in GP2 to adjacent loop in GP2, preferably by introduction of two additional cysteine residues, preferably at positon 370 or 369 and 386 or 385, preferably by amino acid substitutions W3700 or W369C and W386C or W385C,
B amino acid substitutions allowing stabilization of GP2, preferably by introduction of a helix-breaking point mutation in the metastable region of HR1 of GP2 subunit, more preferably by introducing E329P or E328P,
C replacing the native GP1-GP2 cleavage site (S1 P) with a furin cleavage site, e.g. RRLL to RRRR to facilitate efficient processing of the mRNA encoded antigenic protein in target cells, preferably by amino acid substitutions L258R, L259R or L257R, L258R,
wherein prefusion-stabilized GPC preferably comprises at least one mutation A selected from A1, A2 and A3.

6. RNA according to any one of the preceding claims, wherein
said at least one coding sequence encodes at least one antigenic peptide or protein derived from LASV GPC comprising or consisting of an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 1-254 or a fragment or a variant thereof;
said at least one coding sequence encoding at least one antigenic peptide or protein derived from LASV prefusion-stabilized GPC (“GPCmut1” or “GPCstabilized”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 3567-3820 or a fragment or variant of any of these sequences;
said at least one coding sequence encoding at least one antigenic peptide or protein derived from LASV prefusion-stabilized GPC (“GPCmut2”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 22949-22980 or a fragment or variant of any of these sequences;
said at least one coding sequence encoding at least one antigenic peptide or protein derived from LASV prefusion-stabilized GPC (“GPCmut3”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 24645-24676 or a fragment or variant of any of these sequences;
said at least one coding sequence encoding at least one antigenic peptide or protein derived from LASV prefusion-stabilized GPC (“GPCmut4”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 26341-26372 or a fragment or variant of any of these sequences;
said at least one coding sequence encoding at least one antigenic peptide or protein derived from LASV prefusion-stabilized GPC (“GPCmut5”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 28037-28068 or a fragment or variant of any of these sequences;
said at least one coding sequence encoding at least one antigenic peptide or protein derived from LASV prefusion-stabilized GPC (“GPCmut6”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 29733-29764 or a fragment or variant of any of these sequences;
said at least one coding sequence encoding at least one antigenic peptide or protein derived from LASV prefusion-stabilized GPC (“GPCmut7”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 31429-31460 or a fragment or variant of any of these sequences;
said at least one coding sequence encoding at least one antigenic peptide or protein derived from LASV prefusion-stabilized GPC (“GPCmut8”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 33125-33156 or a fragment or variant of any of these sequences;
said at least one coding sequence encoding at least one antigenic peptide or protein derived from LASV prefusion-stabilized GPC (“GPCmut9”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 34821-34852 or a fragment or variant of any of these sequences;
said at least one coding sequence encoding at least one antigenic peptide or protein derived from LASV prefusion-stabilized GPC (“GPCmut10”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 36517-36548 or a fragment or variant of any of these sequences;
said at least one coding sequence encoding at least one antigenic peptide or protein derived from LASV prefusion-stabilized GPC (“GPCmut11”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 38213-38244 or a fragment or variant of any of these sequences,
said at least one coding sequence encoding at least one antigenic peptide or protein derived from LASV prefusion-stabilized GPC (“GPCmut12”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 39909-39940 or a fragment or variant of any of these sequences,
said at least one coding sequence encoding at least one antigenic peptide or protein derived from LASV GPC lacking the cytoplasmic tail (“GPCmut13”) comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 41605- 41636 or a fragment or variant of any of these sequences,
said at least one coding sequence encodes at least one antigenic peptide or protein derived from LASV NP comprising or consisting of an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 7547-7797 or a fragment or a variant thereof;
said at least one coding sequence encoding at least one antigenic peptide or protein derived from HsPLAT_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 17968-18001 or a fragment or variant of any of these sequences;
said at least one coding sequence encoding at least one antigenic peptide or protein derived from HsALB_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 19328-19361 or a fragment or variant of any of these sequences;
said at least one coding sequence encoding at least one antigenic peptide or protein derived from IgE_NP comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 20689-20722 or a fragment or variant of any of these sequences;
said at least one coding sequence encodes at least one antigenic peptide or protein derived from LASV Z comprising or consisting of an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 11166-11347 or a fragment or a variant thereof.

7. RNA according to any one of the preceding claims, wherein
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 255-2286 or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 3821-6106 (encoding GPCmut1) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 22981-23108 (encoding GPCmut2) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 24677-24804 (encoding GPCmut3) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 26373-26500 (encoding GPCmut4) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 28069-28196 (encoding GPCmut5) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 29765-29892 (encoding GPCmut6) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 31461-31588 (encoding GPCmut7) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 33157-33284 (encoding GPCmut8) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 34853-34980 (encoding GPCmut9) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 36549-36676 (encoding GPCmut10) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 38245-38372 (encoding GPCmut11) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 39941-40068 (encoding GPCmut12) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 41637-41764 (encoding GPCmut13) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 7798-9805 (encoding NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 18002-18103 (encoding SP- HsPLAT_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 19362-19463 (encoding SP- HsALB_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 20723-20824 (encoding SP- IgE_NP) or a fragment or a fragment or variant of any of these sequences;
said the at least one coding sequence comprises or consists of at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 11348-12803 (encoding Z) or a fragment or a fragment or variant of any of these sequences.

8. RNA according to any one of the preceding claims, wherein the at least one coding sequence is a codon modified coding sequence, wherein the amino acid sequence encoded by the at least one codon modified coding sequence is preferably not being modified compared to the amino acid sequence encoded by the corresponding wild type coding sequence.

9. RNA according to claim 8, wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

10. RNA according to claim 8 or 9, wherein the
at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to any one SEQ ID NOs: 509-2286 (encoding GPC) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 4075-6106 (encoding GPCmut1) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 22981-23108 (encoding GPCmut2) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 24677-24804 (encoding GPCmut3) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 26373-26500 (encoding GPCmut4) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 28069-28196 (encoding GPCmut5) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 29765-29892 (encoding GPCmut6) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 31461-31588 (encoding GPCmut7) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 33157-33284 (encoding GPCmut8) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 34853-34980 (encoding GPCmut9) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 36549-36676 (encoding GPCmut10) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 38245-38372 (encoding GPCmut11) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 39941-40068 (encoding GPCmut12) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 41637-41764 (encoding GPCmut13) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 8049-9805 (encoding NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 18002-18103, 19362-19463, or 20723-20824 (encoding SP-NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 11530-12803 (encoding Z) or a fragment or variant of any of these sequences.

11. RNA according to claims 8 to 10, wherein the
at least one coding sequence comprising a G/C optimized or G/C content modified coding sequence (opt1, opt5, opt6, opt11) comprising a nucleic acid sequence which is identical or at least 50%, 60%, 70%, 80%, 85%, 86%, 87%, 88%, 89%, 90%, 91%, 92%, 93%, 94%, 95%, 96%, 97%, 98%, or 99% identical to any one of the nucleic acid sequences according to the SEQ ID NOs: 509-762, 1525-2286 (encoding GPC) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 4075-4582, 5345-6106 (encoding GPCmut1) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 22981-23044, 23077-23108 (encoding GPCmut2) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 24677-24740, 24773-24804 (encoding GPCmut3) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 26373-26436, 26469-26500 (encoding GPCmut4) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 28069-28132, 28165-28196 (encoding GPCmut5) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 29765-29828, 29861-29892 (encoding GPCmut6) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 31461-31524, 31557-31588 (encoding GPCmut7) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 33157-33220, 33253-33284 (encoding GPCmut8) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 34853-34916, 34949-34980 (encoding GPCmut9) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 36549-36612, 36645-36676 (encoding GPCmut10) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 38245-38308, 38341-38372 (encoding GPCmut11) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 39941-40004, 40037-40068 (encoding GPCmut12) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 41637-41700, 41733-41764 (encoding GPCmut13) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 8049-8299, 9053-9805 (encoding NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 18002-18035, 18070-18103, 19362-19395, 19430-19463, 20723-20756, 20791-20824 (encoding SP_NP) or a fragment or variant of any of these sequences, or
at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 11530-11711, 12258-12803 (encoding Z) or a fragment or variant of any of these sequences.

12. RNA according to any one of the preceding claims, wherein the RNA comprises a 5′-cap structure, preferably m7G(5′), m7G(5′)ppp(5′)(2′OMeA), or m7G(5′)ppp(5′)(2′OMeG), wherein cap1 (m7G(5′)ppp(5′)(2′OMeA), or m7G(5′)ppp(5′)(2′OMeG) is particularly preferred.

13. RNA according to any one of the preceding claims, wherein the RNA comprises at least one poly(A) sequence, preferably comprising 30 to 200 adenosine nucleotides, preferably about 64 adenosine nucleotides (A64), about 100 adenosine nucleotides (A100) or about 150 adenosine nucleotides.

14. RNA according to any one of the preceding claims, wherein the RNA comprises at least one histone stem-loop, wherein the histone stem-loop preferably comprises a nucleic acid sequence according to SEQ ID NOs: 13842 or 13843 or a fragment or variant thereof.

15. RNA according to any one of the preceding claims comprising the following elements:
a) 5′-cap structure, preferably as defined in claim 12;
b) 5′-UTR and/or 3′-UTR as defined in claims 1 to 3;
c) at least one coding sequence, preferably as defined by any one of claims 7 to 11 or encoding a protein as defined by any one of claims 4 to 6 or;
d) optionally, a poly(A) sequence, preferably as defined by claim 13;
e) optionally, a poly(C) sequence, preferably as defined by claim 13;
f) optionally, a histone stem-loop, preferably as defined by any one of claim 14;
g) optionally, a 3′-terminal sequence according to SEQ ID NOs: 13848-13867, 13873-13879.

16. RNA according claims to any one of the preceding claims comprising the following elements, preferably in 5′- to 3′-direction:
a) 5′-cap structure, preferably as defined in claim 12;
b) 5′-UTR and/or 3′-UTR according to a-1, a-2, b-4, i-2, or i-3;
c) at least one coding sequence as defined by any one of claims 7 to 11 wherein said coding sequence is located between said 5′-UTR and said 3′-UTR, preferably downstream of said 5′-UTR and upstream of said 3′-UTR;
d) optionally, a poly(A) sequence, preferably as defined by claim 13;
e) optionally, poly(C) sequence, preferably as defined by claim 13;
f) optionally, histone stem-loop, preferably as defined by any one of claim 14;
g) optionally, a 3′-terminal sequence according to SEQ ID NOs: 13848-13867, 13873-13879.

17. RNA according to any one of the preceding claims wherein
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2287-3566, 14056-15207 (encoding GPC) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 6107-7546, 15208-16743 (encoding GPCmut1) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 23109-24644 (encoding GPCmut2) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 24805-26340 (encoding GPCmut3) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 26501-28036 (encoding GPCmut4) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 28197-29732 (encoding GPCmut5) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 29893-31428 (encoding GPCmut6) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 31589-33124 (encoding GPCmut7) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 33285-34820 (encoding GPCmut8) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 34981-36516 (encoding GPCmut9) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 36677-38212 (encoding GPCmut10) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 38373-39908 (encoding GPCmut11) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 40069-41604 (encoding GPCmut12) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 41765-43300 (encoding GPCmut13) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 9806-11165, 16744-17967 (encoding NP) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 18104-19327, 19464- 20687, 20825-22048 (encoding SP-NP) or a fragment or variant of any of these sequences;
said RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 12804-13803, 22049-22948 (encoding Z) or a fragment or variant of any of these sequences.

18. A composition comprising at least one RNA as defined in any one of claims 1 to 17, wherein the composition optionally comprises at least one pharmaceutically acceptable carrier.

19. Composition according to claim 18, wherein the composition comprises more than one or a plurality, preferably 2, 3, 4, 5, 6, 7, 8, 9, 10, 11, 12, 13, 14, 15 of different RNAs each defined in any one of claims 1 to 18.

20. Composition according to claim 19, wherein each of the different RNAs encodes a different antigenic peptide or protein derived from the same LASV, or wherein each of the RNAs encodes a different antigenic peptide or protein derived from different proteins of the same LASV, or wherein each of the RNAs encodes a different antigenic peptide or protein derived from different proteins of different LASV.

21. Composition according to claim 19 or 20, wherein the composition comprises at least one RNA encoding at least one antigenic peptide or protein derived from GPC or prefusion-stabilized GPC or a variant or fragment thereof and at least one additional RNA encoding at least one antigenic peptide or protein derived from NP (or SP-NP) or a variant or fragment thereof, and/or at least one additional RNA encoding at least one antigenic peptide or protein derived from Z or a variant or fragment thereof.

22. Composition according to claims 19 to 21, wherein the composition comprises at least one RNA encoding at least one antigenic peptide or protein derived from GPC or prefusion-stabilized GPC or a variant or fragment thereof and at least one additional RNA encoding at least one antigenic peptide or protein derived from NP (or SP-NP) or a variant or fragment thereof.

23. Composition according to claims 19 to 22, wherein said antigenic peptides or proteins are derived from the same LASV or from different LASV or combinations thereof.

24. Composition according to claim 23, wherein the different LASV belong to different LASV clades or different LASV lineages, preferably to the LASV clades I, II, III, IV, V, and VI or to the LASV lineages I, II, III, IV, V, and VI.

25. Composition according to claims 18 to 24, wherein at least one RNA is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

26. Composition according to claim 25, wherein the at least one RNA is complexed or associated with one or more lipids, thereby forming liposomes, lipid nanoparticles, lipoplexes, and/or nanoliposomes.

27. Composition according to claim 26, wherein the at least one RNA is complexed with one or more lipids thereby forming lipid nanoparticles (LNP).

28. Composition according to claim 26 or 27, wherein the LNP comprises a cationic lipid with the formula III: 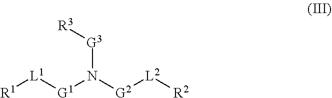
or a pharmaceutically acceptable salt, tautomer, prodrug or stereoisomer thereof, wherein:
L¹ or L² is each independently —O(C═O)—, —(C═O)O—, —C(═O)—, —O—, —S(O)ₓ—, —S—S—, —C(═O)S—, —SC(═O)—, —NR^{a}C(═O)—, —C(=O)NR^{a}—, —NR^{a}C(═O)NR^{a}—, —OC(=O)NR^{a}— or —NR^{a}C(═O)O—, preferably L¹ or L² is —O(C═O)—or
—(C═O)O—;
G¹ and G² are each independently unsubstituted C₁-C₁₂ alkylene or C₁-C₁₂ alkenylene;
G³ is C₁-C₂₄ alkylene, alkenylene, C₃-C₈ cycloalkylene, or C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2;

29. Composition according to claim 28, wherein the cationic lipid is a compound of formula III, and wherein:
L¹ and L² are each independently —O(C═O)— or (C═O)—O—;
G³ is C₁-C₂₄ alkylene or C₁-C₂₄ alkenylene; and
R³ is H or OR⁵.

30. Composition according to any one of claims 28 to 29, wherein the cationic lipid is a compound of formula III, and wherein:
L¹ and L² are each independently —O(C═O)— or (C═O)—O—; and
R¹ and R² each independently have one of the following structures:

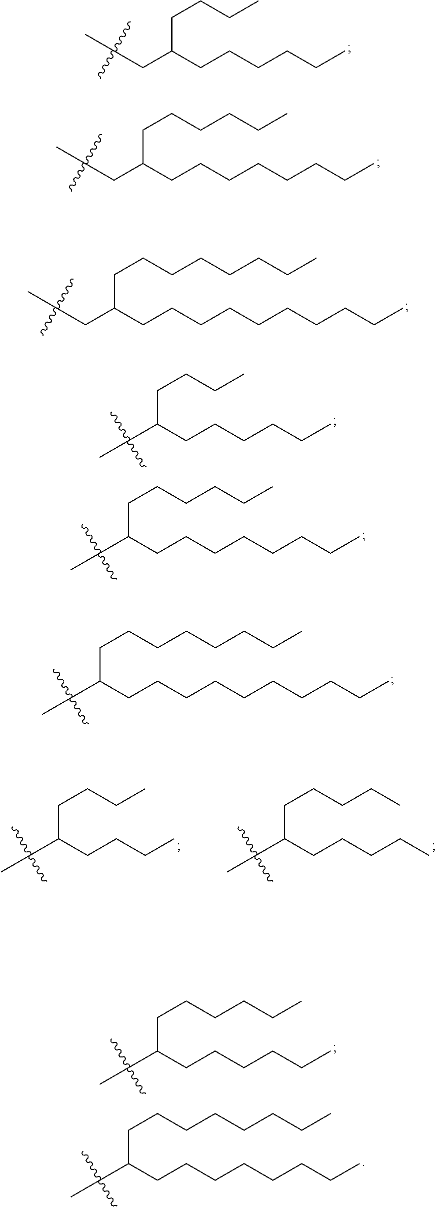

31. Composition according to any one of claims 28 to 30, wherein the cationic lipid is a compound of formula III, and wherein R³ is OH.

32. Composition according to any one of claims 28 to 31, wherein the cationic lipid is selected from structures III-1 to III-36:

| No. | Structure |
|---|---|
| III-1 | 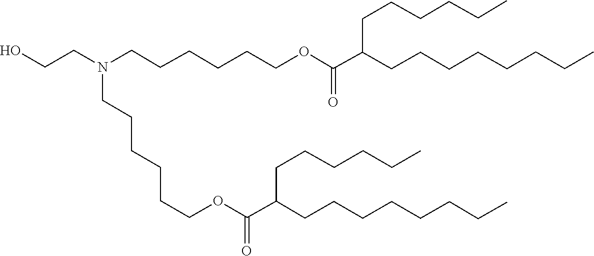 [SMILES: CCCCCCCCC(CCCCCC)C(=O)OCCCCCCN(CCO)CCCCCCOC(=O)C(CCCCCC)CCCCCCCC] |
| III-2 | 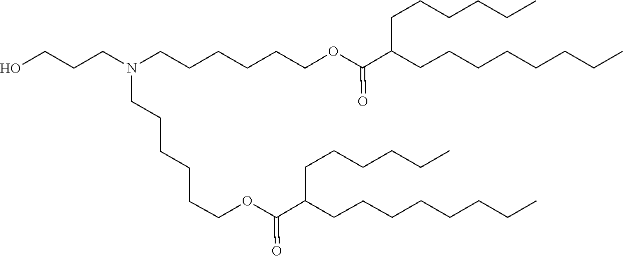 [SMILES: CCCCCCCCC(CCCCCC)C(=O)OCCCCCCN(CCCO)CCCCCCOC(=O)C(CCCCCC)CCCCCCCC] |
| III-3 | 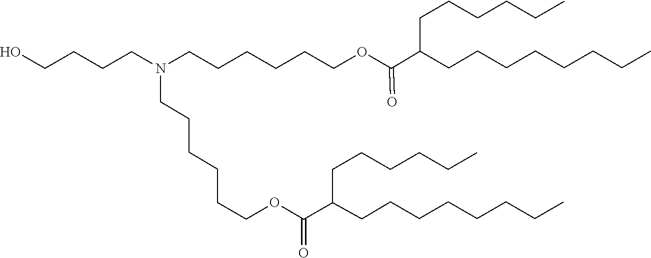 [SMILES: CCCCCCCCC(CCCCCC)C(=O)OCCCCCCN(CCCCO)CCCCCCOC(=O)C(CCCCCC)CCCCCCCC] |
| III-4 | 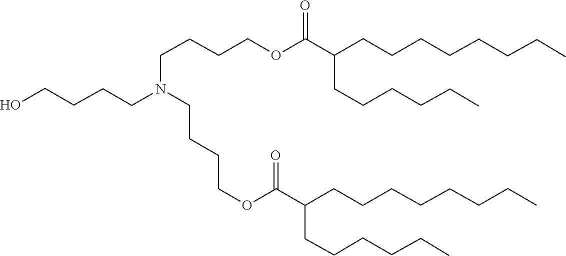 [SMILES: CCCCCCCCC(CCCCCC)C(=O)OCCCCN(CCCCO)CCCCOC(=O)C(CCCCCC)CCCCCCCC] |
| III-5 | 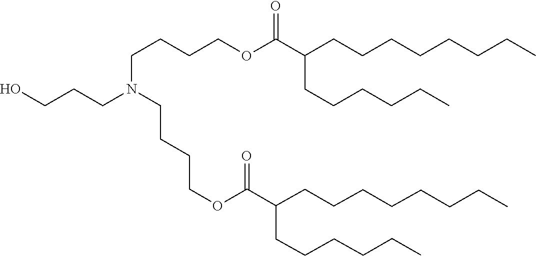 [SMILES: CCCCCCCCC(CCCCCC)C(=O)OCCCCN(CCCO)CCCCOC(=O)C(CCCCCC)CCCCCCCC] |
| III-6 | 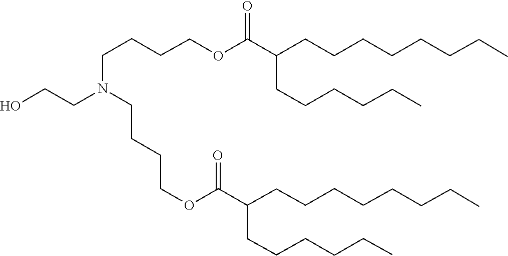 [SMILES: CCCCCCCCC(CCCCCC)C(=O)OCCCCN(CCO)CCCCOC(=O)C(CCCCCC)CCCCCCCC] |
| III-7 | 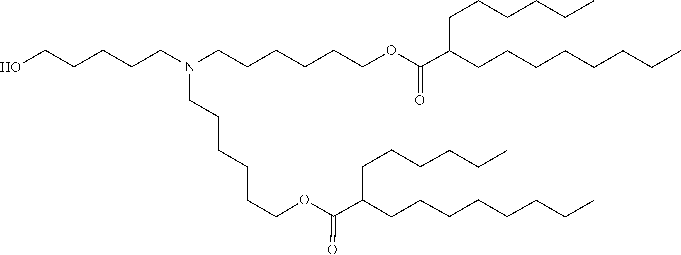 [SMILES: CCCCCCCCC(CCCCCC)C(=O)OCCCCCCN(CCCCCO)CCCCCCOC(=O)C(CCCCCC)CCCCCCCC] |
| III-8 | 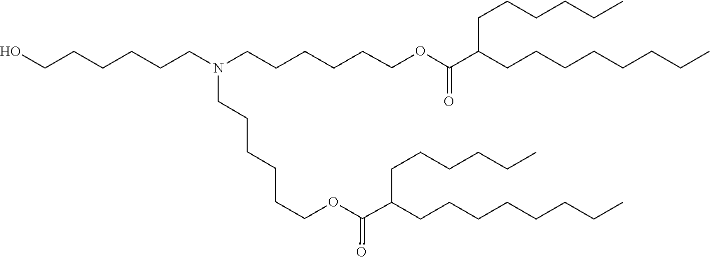 [SMILES: CCCCCCCCC(CCCCCC)C(=O)OCCCCCCN(CCCCCCO)CCCCCCOC(=O)C(CCCCCC)CCCCCCCC] |
| III-9 | 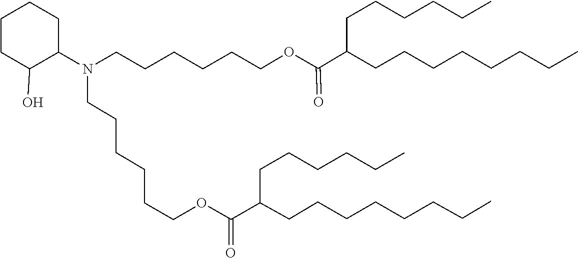 [SMILES: CCCCCCCCC(CCCCCC)C(=O)OCCCCCCN(CCCCCCOC(=O)C(CCCCCC)CCCCCCCC)C1CCCCC1O] |
| III-10 | 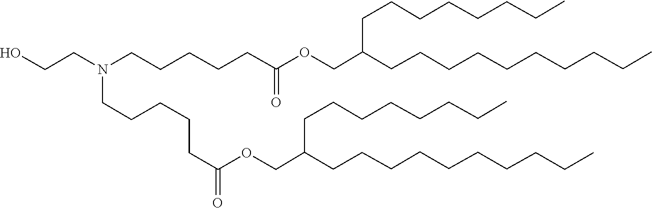 [SMILES: CCCCCCCCCCC(CCCCCCCC)COC(=O)CCCCCN(CCO)CCCCCC(=O)OCC(CCCCCCCC)CCCCCCCCCC] |
| III-11 | 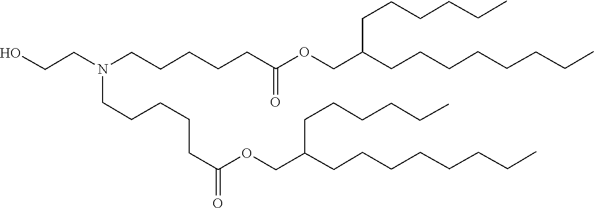 [SMILES: CCCCCCCCC(CCCCCC)COC(=O)CCCCCN(CCO)CCCCCC(=O)OCC(CCCCCC)CCCCCCCC] |
| III-12 | 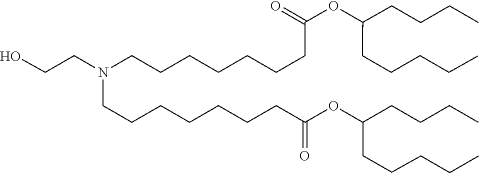 [SMILES: CCCCCC(CCCC)OC(=O)CCCCCCCN(CCO)CCCCCCCC(=O)OC(CCCC)CCCCC] |
| III-13 | 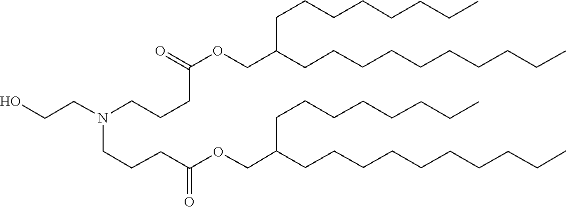 [SMILES: CCCCCCCCCCC(CCCCCCCC)COC(=O)CCCN(CCO)CCCC(=O)OCC(CCCCCCCC)CCCCCCCCCC] |
| III-14 | 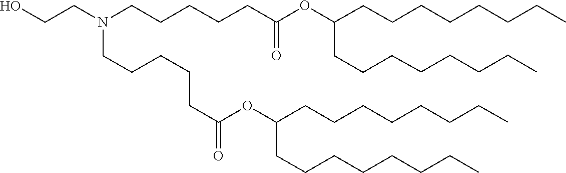 [SMILES: CCCCCCCCC(CCCCCCCC)OC(=O)CCCCCN(CCO)CCCCCC(=O)OC(CCCCCCCC)CCCCCCCC] |
| III-15 | 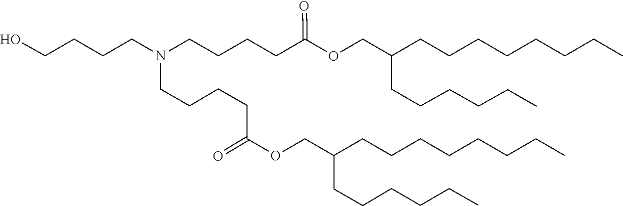 [SMILES: CCCCCCCCC(CCCCCC)COC(=O)CCCCN(CCCCO)CCCCC(=O)OCC(CCCCCC)CCCCCCCC] |
| III-16 | 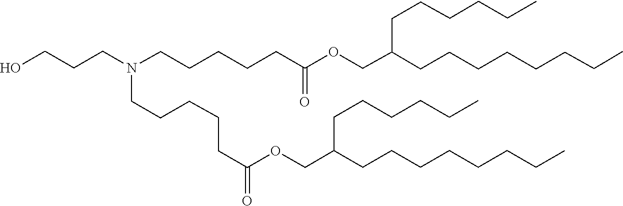 [SMILES: CCCCCCCCC(CCCCCC)COC(=O)CCCCCN(CCCO)CCCCCC(=O)OCC(CCCCCC)CCCCCCCC] |
| III-17 | 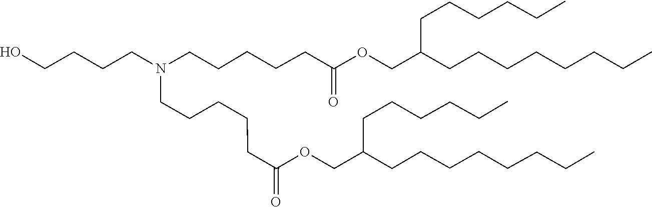 [SMILES: CCCCCCCCC(CCCCCC)COC(=O)CCCCCN(CCCCO)CCCCCC(=O)OCC(CCCCCC)CCCCCCCC] |
| III-18 | 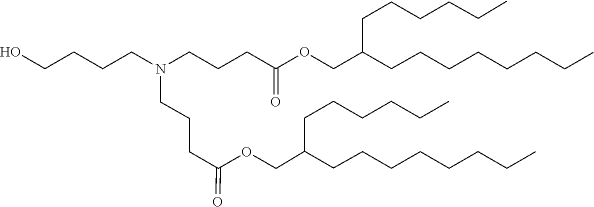 [SMILES: CCCCCCCCC(CCCCCC)COC(=O)CCCN(CCCCO)CCCC(=O)OCC(CCCCCC)CCCCCCCC] |
| III-19 | 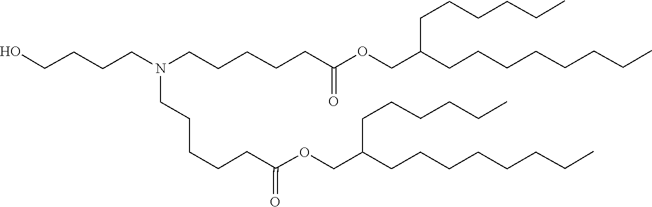 [SMILES: CCCCCCCCC(CCCCCC)COC(=O)CCCCCN(CCCCO)CCCCCC(=O)OCC(CCCCCC)CCCCCCCC] |
| III-20 | 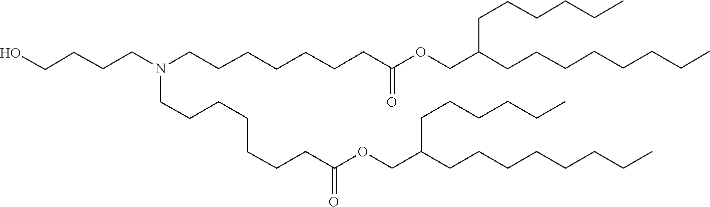 [SMILES: CCCCCCCCC(CCCCCC)COC(=O)CCCCCCCN(CCCCO)CCCCCCCC(=O)OCC(CCCCCC)CCCCCCCC] |
| III-21 | 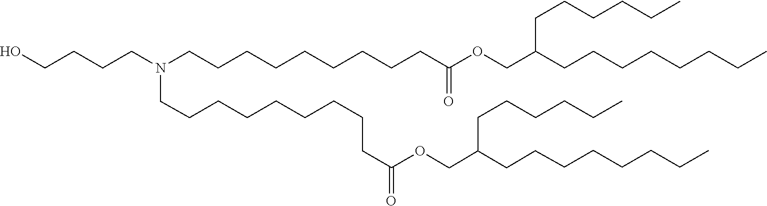 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 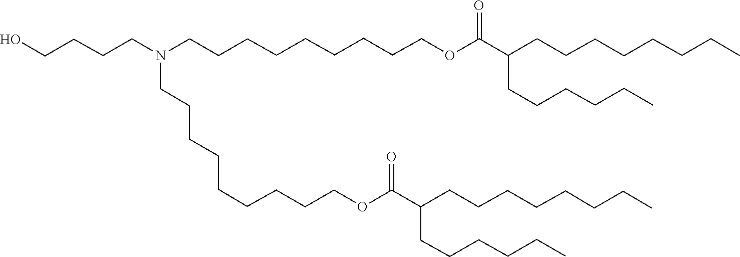 [SMILES: CCCCCCCCC(CCCCCC)C(=O)OCCCCCCCCCN(CCCCO)CCCCCCCCCOC(=O)C(CCCCCC)CCCCCCCC] |
| III-23 | 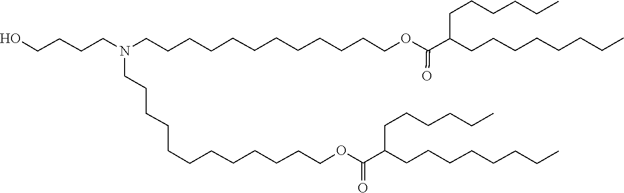 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 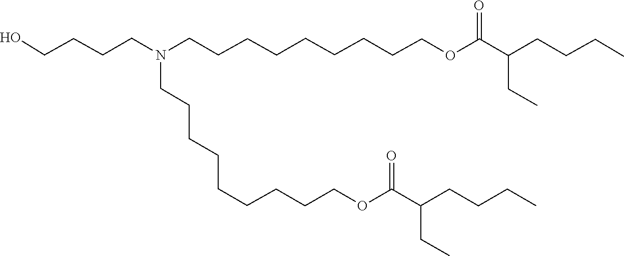 [SMILES: CCCCC(CC)C(=O)OCCCCCCCCCN(CCCCO)CCCCCCCCCOC(=O)C(CC)CCCC] |
| III-25 | 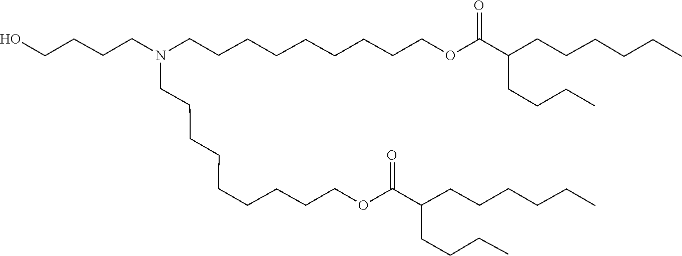 [SMILES: CCCCCCC(CCCC)C(=O)OCCCCCCCCCN(CCCCO)CCCCCCCCCOC(=O)C(CCCC)CCCCCC] |
| III-26 | 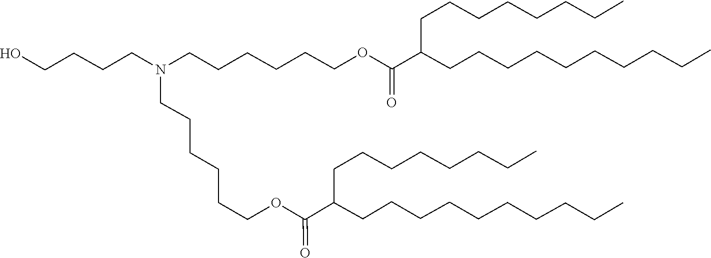 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 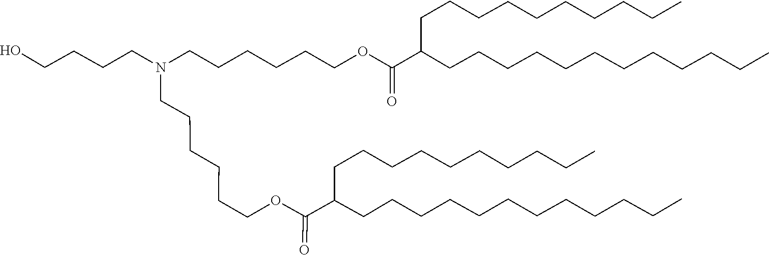 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 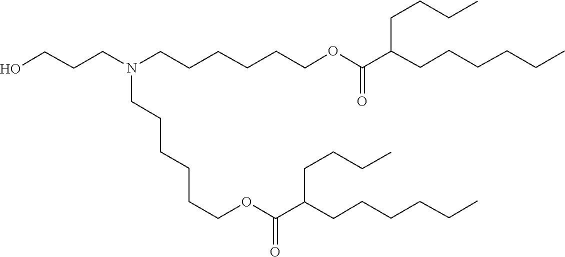 [SMILES: CCCCCCC(CCCC)C(=O)OCCCCCCN(CCCO)CCCCCCOC(=O)C(CCCC)CCCCCC] |
| III-29 | 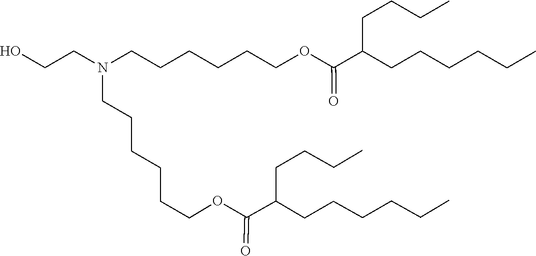 [SMILES: CCCCCCC(CCCC)C(=O)OCCCCCCN(CCO)CCCCCCOC(=O)C(CCCC)CCCCCC] |
| III-30 | 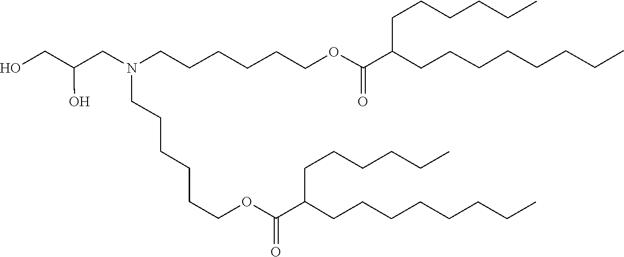 [SMILES: CCCCCCCCC(CCCCCC)C(=O)OCCCCCCN(CCCCCCOC(=O)C(CCCCCC)CCCCCCCC)CC(O)CO] |
| III-31 | 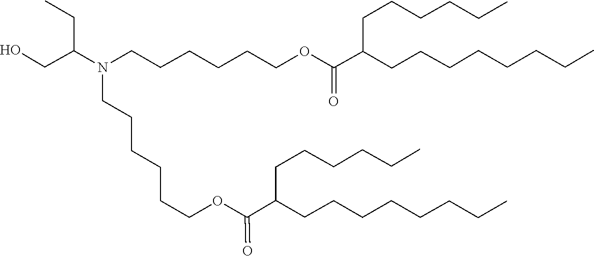 [SMILES: CCCCCCCCC(CCCCCC)C(=O)OCCCCCCN(CCCCCCOC(=O)C(CCCCCC)CCCCCCCC)C(CC)CO] |
| III-32 | 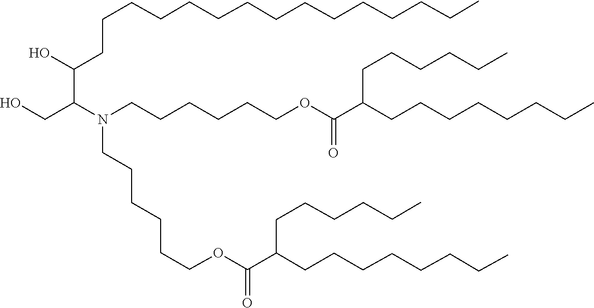 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 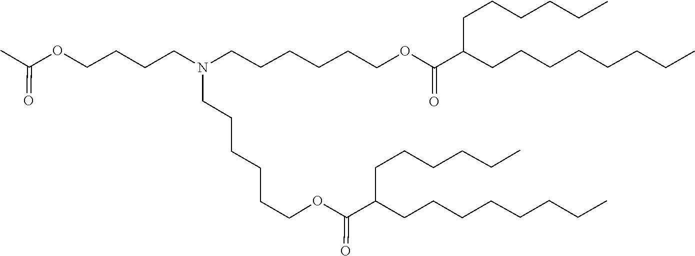 [SMILES: CCCCCCCCC(CCCCCC)C(=O)OCCCCCCN(CCCCCCOC(=O)C(CCCCCC)CCCCCCCC)CCCCOC(C)=O] |
| III-34 | 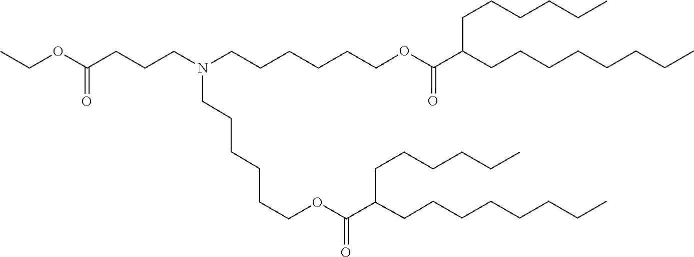 [SMILES: CCCCCCCCC(CCCCCC)C(=O)OCCCCCCN(CCCCCCOC(=O)C(CCCCCC)CCCCCCCC)CCCC(=O)OCC] |
| III-35 | 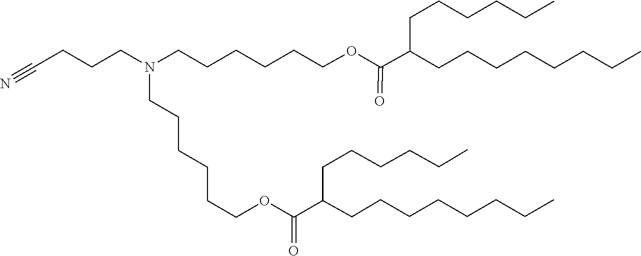 [SMILES: CCCCCCCCC(CCCCCC)C(=O)OCCCCCCN(CCCC#N)CCCCCCOC(=O)C(CCCCCC)CCCCCCCC] |
| III-36 | 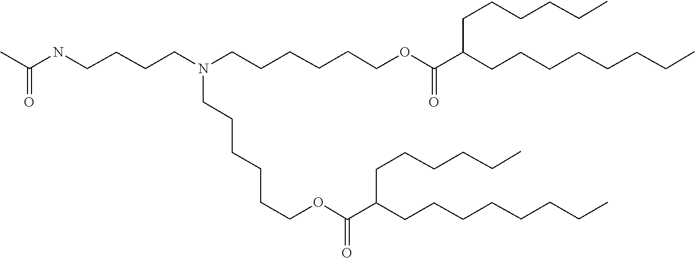 |

33. Composition according to any one of claims 28 to 32, wherein the cationic lipid is

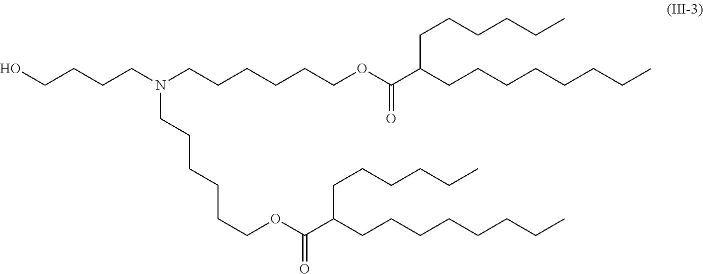 [SMILES: CCCCCCCCC(CCCCCC)C(=O)OCCCCCCN(CCCCO)CCCCCCOC(=O)C(CCCCCC)CCCCCCCC]

34. Composition according to any one of claims 27 to 33, wherein the LNP comprises a PEG lipid with the formula (IV): 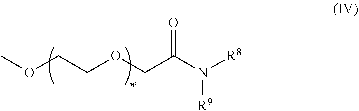
wherein R⁸ and R⁹ are each independently a straight or branched, saturated or unsaturated alkyl chain containing from 10 to 30 carbon atoms, wherein the alkyl chain is optionally interrupted by one or more ester bonds;
and w has a mean value ranging from 30 to 60.

35. Composition according to claim 34, wherein in the PEG lipid R⁸ and R⁹ are saturated alkyl chains.

36. Composition according to claim 34 or 35, wherein the PEG lipid is 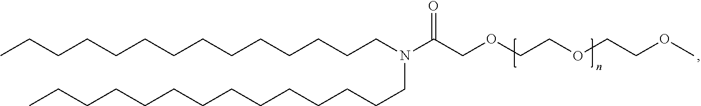
wherein n has a mean value ranging from 30 to 60, preferably wherein n has a mean value of about 45, 46, 47, 48, 49, 50, 51, 52, 53, 54, most preferably wherein n has a mean value of 49.

37. Composition according to any one of claims 27 to 36, wherein the LNP comprises one or more neutral lipids and/or a steroid or steroid analogues.

38. Composition according to claim 37, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE).

39. Composition according to claim 37 or 38, wherein the neutral lipid is 1,2-distearoyl-sn-glycero-3-phosphocholine (DSPC), and wherein the molar ratio of the cationic lipid to DSPC is optionally in the range from about 2:1 to 8:1.

40. Composition according to claim 37, wherein the steroid is cholesterol, and wherein the molar ratio of the cationic lipid to cholesterol is optionally in the range from about 2:1 to 1:1

41. Composition according to any one of claims 27 to 40, wherein the LNP essentially consists of
(i) at least one cationic lipid, preferably as defined in any one of claims 28 to 33;
(ii) a neutral lipid, preferably as defined in any one of claims 37 to 39;
(iii) a steroid or steroid analogue, preferably as defined in claim 40; and
(iv) a PEG-lipid, e.g. PEG-DMG or PEG-cDMA, preferably as defined in any one of claims 34 to 36,
wherein (i) to (iv) are in a molar ratio of about 20-60% cationic lipid, 5-25% neutral lipid, 25-55% sterol, and 0.5-15% PEG-lipid.

42. A vaccine comprising at least one RNA as defined in any one of claims 1 to 17, or the composition as defined in any one of claims 18 to 41.

43. Vaccine according to claim 42, wherein the at least one RNA as defined in any one of claims 1 to 17, or the composition as defined in any one of claims 18 to 41 elicits an adaptive immune response.

44. A Kit or kit of parts comprising at least one RNA as defined in any one of claims 1 to 17, the composition as defined in any one of claims 18 to 41, and/or the vaccine as defined in any one of claim 42 or 43, optionally comprising a liquid vehicle for solubilising, and optionally technical instructions providing information on administration and dosage of the components.

45. Kit or kit of parts according to claim 44 comprising at least the following components
a) at least one RNA as defined in any one of claims 1 to 17 encoding at least one antigenic peptide or protein derived from a LASV, preferably derived from GPC or prefusion-stabilized GPC, wherein said RNA is preferably complexed with one or more lipids thereby forming lipid nanoparticles (LNP); and
b) at least one, two or more further RNA species as defined in any one of claims 1 to 17 each encoding an antigenic peptide or protein derived from a LASV, preferably derived from NP or Z, wherein each of said further RNA species are preferably complexed with one or more lipids thereby forming lipid nanoparticles (LNP),
wherein components a) and b) are provided as separate entities or as a single entity.

46. Kit or kit of parts according to claim 45 or 46 further comprising Ringer lactate solution.

47. RNA as defined in any one of claims 1 to 17, the composition as defined in any one of claims 18 to 41, the vaccine as defined in any one of claim 42 or 43, or the kit or kit of parts as defined in claims 44 to 46 for use as a medicament.

48. RNA as defined in any one of claims 1 to 17, the composition as defined in any one of claims 18 to 41, the vaccine as defined in any one of claim 42 or 43, or the kit or kit of parts as defined in claims 44 to 46 for use in the treatment or prophylaxis of an infection with a virus, preferably with LASV, or a disorder related to such an infection.

49. A method of treating or preventing an infection with a LASV, or a disorder related to such an infection, wherein the method comprises applying or administering to a subject in need thereof the at least one RNA as defined in any one of claims 1 to 17, the composition as defined in any one of claims 18 to 41, the vaccine as defined in any one of claim 42 or 43, or the kit or kit of parts as defined in claim 44 to 46.
